(19) United States
(12) Patent Application Publication
(10) Pub. No.: US 2025/0304537 A1
(43) Pub. Date: Oct. 2, 2025

(54) HETEROARENES, PHARMACEUTICAL COMPOSITIONS CONTAINING THE SAME, AND METHODS OF USING THE SAME

(71) Applicant: Repare Therapeutics Inc., St-Laurent (CA)

(72) Inventors: Cameron BLACK, St-Laurent (CA); Janek SZYCHOWSKI, Montreal (CA); Bingcan LIU, Montreal (CA); Evelyne DIETRICH, Laval (CA); Frédéric VALLÉE, Montreal (CA); Simon SURPRENANT, Boisbriand (CA); Alexander PERRYMAN, Pointe-Claire (CA); Sheldon N. CRANE, Notre Dame de l'lle Perrot (CA); Vouy Linh TRUONG, Pierrefonds (CA); Alexanne BOUCHARD, Montreal (CA); Abbas ABDOLI, Montreal (CA); Frederic IZQUIERDO, Lachine (CA); Dominique BELLA NDONG, L'île-Perrot (CA); Stephane CIBLAT, Montreal (CA); Francis BARABE, Montreal (CA); Thomas PINTER, Montreal (CA)

(21) Appl. No.: 18/866,842

(22) PCT Filed: May 18, 2023
(86) PCT No.: PCT/CA2023/050691

§ 371 Date: Nov. 18, 2024

Related U.S. Application Data
(60) Provisional application No. 63/343,365, filed on May 18, 2022.

(51) Int. Cl. C07D 239/42; A61K 31/416; A61K 31/425; A61K 31/426; A61K 31/44; A61K 31/443; A61K 31/4439; A61K 31/444; A61K 31/47; A61K 31/4725; A61K 31/496; A61K 31/4965; A61K 31/497; A61K 31/50; A61K 31/501; A61K 31/505; A61K 31/506; A61K 31/5377; A61K 31/538; A61K 31/541; C07B 59/00; C07D 213/56; C07D 213/73; C07D 213/81; C07D 215/48; C07D 237/08; C07D 239/26; C07D 241/12; C07D 275/02; C07D 277/30; C07D 401/04; C07D 401/10; C07D 401/14; C07D 403/04; C07D 403/14; C07D 405/04; C07D 405/14; C07D 409/14; C07D 413/04; C07D 413/14; C07D 417/04; C07D 417/14; C07D 491/048; C07D 493/04; C07D 493/08

(52) U.S. Cl. CPC C07D 239/42; A61K 31/416; A61K 31/425; A61K 31/426; A61K 31/44; A61K 31/443; A61K 31/4439; A61K 31/444; A61K 31/47; A61K 31/4725; A61K 31/496; A61K 31/4965; A61K 31/497; A61K 31/50; A61K 31/501; A61K 31/505; A61K 31/506; A61K 31/5377; A61K 31/538; A61K 31/541; C07B 59/002; C07D 213/56; C07D 213/73; C07D 213/81; C07D 215/48; C07D 237/08; C07D 239/26; C07D 241/12; C07D 275/02; C07D 277/30; C07D 401/04; C07D 401/10; C07D 401/14; C07D 403/04; C07D 403/14; C07D 405/04; C07D 405/14; C07D 409/14; C07D 413/04; C07D 413/14; C07D 417/04; C07D 417/14; C07D 491/048; C07D 493/04; C07D 493/08

(57) ABSTRACT

Disclosed are compounds and pharmaceutically acceptable salts thereof that may be used in the treatment of subjects in need thereof. The compounds disclosed herein may be inhibitors of tyrosine and threonine-specific cdc2-inhibitory kinase (Myt1). Also disclosed are pharmaceutical compositions containing the compounds or pharmaceutically acceptable salts thereof and methods of their preparation and use.

### FIELD OF THE INVENTION

The invention relates to compounds and pharmaceutical compositions, their preparation and their use in the treatment of a disease or condition, e.g., cancer, and, in particular, those diseases or conditions (e.g., cancers that harbor CCNE1 amplification/overexpression or FBXW7-mutated cancers) which depend on the activity of membrane-associated tyrosine and threonine-specific cdc2-inhibitory kinase (Myt1).

### BACKGROUND

DNA is continuously subjected to both endogenous insults (e.g., stalled replication forks, reactive oxygen species) and exogenous insults (UV, ionizing radiation, chemical) that can lead to DNA damage. As a result, cells have established sophisticated mechanisms to counteract these deleterious events that would otherwise compromise genomic integrity and lead to genomic instability diseases such as cancer. These mechanisms are collectively referred to as the DNA damage response (DDR). One component of the overall DDR is the activation of various checkpoint pathways that modulate specific DNA-repair mechanisms throughout the various phases of the cell cycle, which includes the G1, S, G2 and Mitosis checkpoints. A majority of cancer cells have lost their G1 checkpoint owing to p53 mutations and as such, rely on the G2 checkpoint to make the necessary DNA damage corrections prior to committing to enter mitosis and divide into 2 daughter cells.

There is a need for new anti-cancer therapeutic approaches, e.g., those utilizing small-molecules, especially therapies allowing for targeted cancer treatment.

### SUMMARY OF THE INVENTION

In an aspect, the invention provides a compound of formula (I):

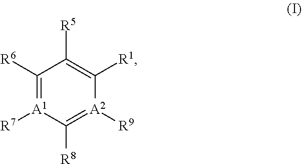

or a pharmaceutically acceptable salt thereof,
where
R¹ is:

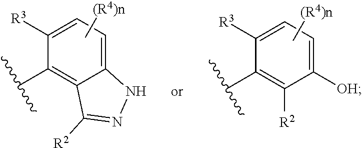

n is 0, 1, or 2;
each of R² and R³ is independently hydrogen, halogen, optionally substituted C₃₋₄ cycloalkyl, or optionally substituted C₁₋₆ alkyl;
each R⁴ is independently halogen;
R⁵ is hydrogen, halogen, hydroxyl, optionally substituted C₁₋₆ alkyl, optionally substituted C₁₋₆ alkoxy, or —N(R^{5A})₂;
each R^{5A} is independently hydrogen, optionally substituted C₁₋₆ alkyl or optionally substituted C₃₋₈ cycloalkyl;
R⁶ is —C(O)NH(R^{6A}), —SO₂R^{6B}, or —C(O)R^{6C};
R^{6A} is hydrogen, optionally substituted C₁₋₆ alkyl, optionally substituted C₃₋₈ cycloalkyl, optionally substituted C₂₋₉ heterocyclyl, optionally substituted C₆₋₁₀ aryl, or optionally substituted C₁₋₉ heteroaryl;
R^{6B} is optionally substituted C₁₋₆ alkyl, optionally substituted C₃₋₈ cycloalkyl, optionally substituted C₆₋₁₀ aryl, or —NH(R^{6A});
R^{6C} is optionally substituted C₁₋₆ alkyl;
each of A¹ and A² is independently N or C;
   R⁷ is absent, hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, or —OR¹⁰; R⁸ is hydrogen, halogen, optionally substituted C₁₋₆ alkyl, optionally substituted C₂₋₆ alkenyl, optionally substituted C₂₋₃ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR¹⁰, —N(R¹¹)₂, or -L-R^{8A}; or R⁷ and R⁸ combine with the atoms to which they are attached to form an optionally substituted C₃₋₈ cycloalkenyl, optionally substituted C₂₋₉ heterocyclyl, optionally substituted C₆₋₁₀ aryl, or optionally substituted C₂₋₁₂ heteroaryl; and R⁹ is absent, hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, or —OR¹⁰;
   or
   R⁸ is hydrogen, halogen, optionally substituted C₁₋₆ alkyl, optionally substituted C₂₋₆ alkenyl, optionally substituted C₂₋₃ alkynyl, optionally substituted C₃₋₈ cycloalkyl, optionally substituted C₃₋₈ cycloalkenyl, optionally substituted C₂₋₉ heterocyclyl, optionally substituted C6-10 aryl, optionally substituted C₁₋₉ heteroaryl, —OR¹⁰, —N(R¹¹)₂, or -L-R^{8A}; and R⁹ is absent, hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, or —OR¹⁰; or R⁸ and R⁹ combine with the atoms to which they are attached to form an optionally substituted C₃₋₈ cycloalkenyl, optionally substituted C₂₋₉ heterocyclyl, optionally substituted C₆₋₁₀ aryl, or optionally substituted C₁₋₁₂ heteroaryl; and R⁷ is absent, hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, or —OR¹⁰;
L is optionally substituted C₂₋₉ heterocyclylene, optionally substituted C₂₋₉ heteroarylene, optionally substituted C₆₋₁₀ arylene, or optionally substituted C₃₋₈ cycloalkylene;
R^{8A} is hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR¹⁰, or —N(R¹¹)₂;
R¹⁰ is hydrogen, optionally substituted C₁₋₆ alkyl, optionally substituted C₁₋₃ heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R¹¹ is independently hydrogen, halogen, optionally substituted C₁₋₆ alkyl, optionally substituted acyl, optionally substituted C₁₋₃ heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl, or —SO₂R^{11A}, or two R¹¹ groups combine to form an optionally substituted C₂₋₉ heterocyclyl; and
each R^{11A} is independently hydrogen, optionally substituted C₁₋₆ alkyl, optionally substituted C₂₋₆ alkenyl, optionally substituted C₂₋₆ alkynyl, optionally substituted C₁₋₃ heteroalkyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl.

In some embodiments, R⁵ is N(R^{5A})₂. In some embodiments, each R^{5A} is hydrogen.

In some embodiments, R¹ is

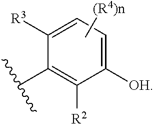

In some embodiments, the compound is of formula (II-A):

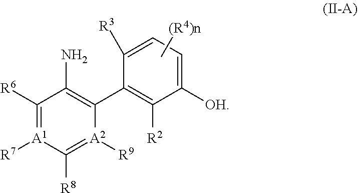

In some embodiments, the compound is of formula (II-A-i):

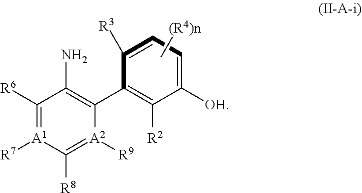

In some embodiments, R¹ is

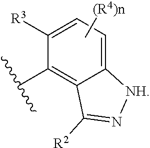

In some embodiments, the compound is of formula (II-B):

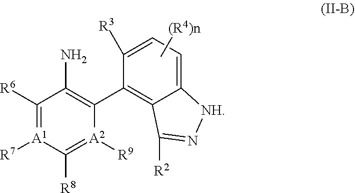

In some embodiments, the compound is of formula (II-B-i):

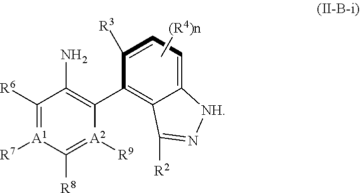

In some embodiments, A¹ is C. In some embodiments, A² is C. In some embodiments, A² is N. In some embodiments, A¹ is N

In some embodiments, R⁷ is hydrogen. In some embodiments, R⁷ and R⁸ combine with the atoms to which they are attached to form an optionally substituted C₂₋₁₂ heteroaryl.

In some embodiments, R⁸ is hydrogen.

In some embodiments, the compound is of formula (II-B-a):

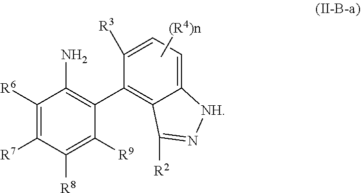

In some embodiments, the compound is of formula (II-B-b):

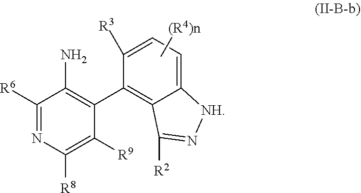

In some embodiments, the compound is of formula (II-B-c):

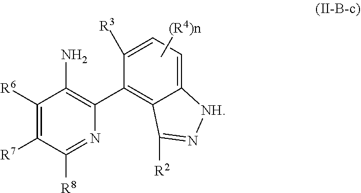

In some embodiments, the compound is of formula (II-B-d):

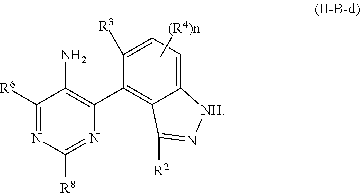

In some embodiments, the compound is of formula (II-A-a):

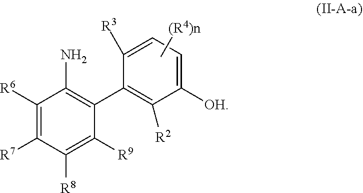

In some embodiments, the compound is of formula (II-A-b):

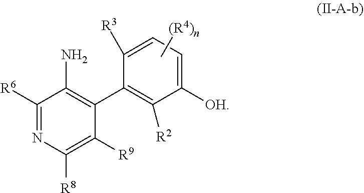

In some embodiments, the compound is of formula (II-A-c):

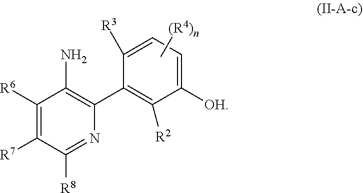

In some embodiments, the compound is of formula (II-A-d):

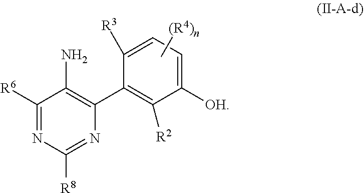

In some embodiments, the compound is of formula (II-C):

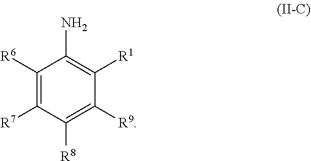

In some embodiments, the compound is of formula (II-D):

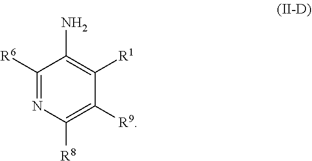

In some embodiments, the compound is of formula (II-E):

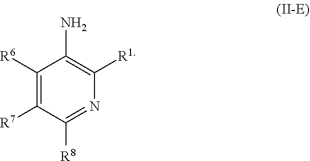

In some embodiments, the compound is of formula (II-F):

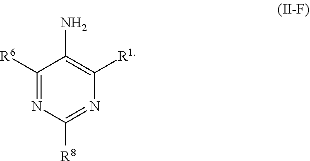

In some embodiments, R⁶ is —C(O)NH(R^{6A}). In some embodiments, each R^{6A} is H.

In some embodiments, R² is H. In some embodiments, R³ is H. In some embodiments, one of R² and R³ is H and the other is optionally substituted C₁₋₆ alkyl. In some embodiments, one of R² and R³ is H and the other is —CH₃. In some embodiments, R² and R³ are each optionally substituted C₁₋₆ alkyl. In some embodiments, R² and R³ are each —CH₃. In some embodiments, R² is halogen. In some embodiments, R² is Cl. In some embodiments, R² is F. In some embodiments, R³ is halogen. In some embodiments, R³ is Cl. In some embodiments, R³ is F.

In some embodiments, n is 0. In some embodiments, n is 1.

In some embodiments, R⁴ is halogen. In some embodiments, R⁴ is F.

In some embodiments, R¹ is:

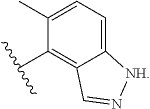 [SMILES: *c1c(C)ccc2[nH]ncc12]

In some embodiments, R⁸ is optionally substituted C₆₋₁₀ aryl. In some embodiments, R⁸ is optionally substituted phenyl. In some embodiments, R⁸ is optionally substituted C₁₋₉ heteroaryl. In some embodiments, R⁸ is -L-R^{8A}.

In some embodiments, L is optionally substituted pyrimidinyl. In some embodiments, L is optionally substituted pyridyl. In some embodiments, L is optionally substituted indazolyl. In some embodiments, L is optionally substituted pyrazolyl. In some embodiments, L is optionally substituted imidazolyl. In some embodiments, L is optionally substituted thiazolyl. In some embodiments, L is optionally substituted pyridazinyl. In some embodiments, L is optionally substituted indolyl. In some embodiments, L is optionally substituted furyl.

In some embodiments, R⁸ is an optionally substituted bicyclic heteroaryl.

In some embodiments, -L-R^{8A} is:

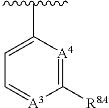

where each of A³ and A⁴ is independently N or CH.

In some embodiments, A³ is N. In some embodiments, A⁴ is N. In some embodiments, A³ is CH. In some embodiments, A⁴ is CH.

In some embodiments, R^{8A} is —OR¹⁰. In some embodiments, R¹⁰ is optionally substituted C₁₋₆ alkyl. In some embodiments, R¹⁰ is —CH₃. In some embodiments, R¹⁰ is optionally substituted C₂₋₉ heterocyclyl. In some embodiments, R¹⁰ is optionally substituted C₆₋₁₀ aryl.

In some embodiments, R^{8A} is —N(R¹¹)₂. In some embodiments, one R¹¹ is H. In some embodiments, one R¹¹ is optionally substituted C₁₋₆ alkyl. In some embodiments, each R¹¹ is H. In some embodiments, two R¹¹ groups combine to form an optionally substituted C₂₋₉ heterocyclyl.

In some embodiments, -L-R^{8A} is:

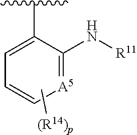

where each R¹⁴ is independently cyano, halogen, optionally substituted C₁₋₆ alkyl, —S(O)₂R^{14A} or optionally substituted C₁₋₈ heteroalkyl;
R^{14A} is optionally substituted C₁₋₆ alkyl;
A⁵ is N or CH; and
p is 0, 1, 2, 3, or 4.

In some embodiments, -L-R^{8A} is

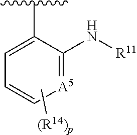

where R¹¹ is optionally substituted acyl.
In some embodiments, -L-R^{8A} is:

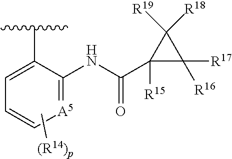

where each of R¹⁵, R¹⁶, R¹⁷, R¹⁸, and R¹⁹ is independently cyano, hydrogen, halogen, —CH₃, —CF₃, or —OCH₃.

In some embodiments, -L-R^{8A} is

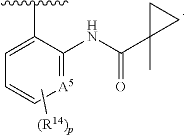

In some embodiments, -L-R^{8A} is

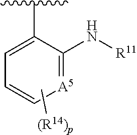

where R¹¹ is optionally substituted C₁₋₉ heteroaryl. In some embodiments, the optionally substituted C₁₋₉ heteroaryl is a 6-membered heteroaryl ring containing at least one nitrogen. In some embodiments, the 6-membered heteroaryl ring contains a total of 2 nitrogen atoms.

In some embodiments, -L-R^{8A} is

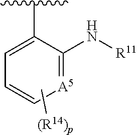

where R¹¹ is:

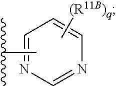

each R^{11B} is independently halogen or optionally substituted C₁₋₆ alkyl; and
q is 0, 1, 2, or 3.

In some embodiments, q is 0. In some embodiments, q is 2.

In some embodiments, -L-R^{8A} is

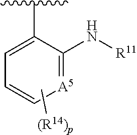

where R¹¹ is optionally substituted C₆₋₁₀ aryl. In some embodiments, the optionally substituted C₆₋₁₀ aryl is optionally substituted phenyl.

In some embodiments, -L-R^{8A} is

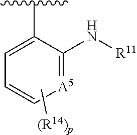

where R¹¹ is optionally substituted C₂₋₉ heterocyclyl.

In some embodiments, -L-R^{8A} is

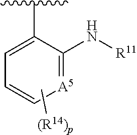

where R¹¹ is —S(O)₂R^{11A}.

In some embodiments, p is 0. In some embodiments, p is 1. In some embodiments, p is 2.

In some embodiments, each R¹⁴ is independently halogen. In some embodiments, each R¹⁴ is F.

In some embodiments, A⁵ is CH. In some embodiments, A⁵ is N.

In some embodiments, -L-R^{8A} is:

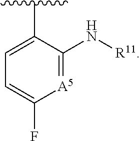

In some embodiments, R⁸ is optionally substituted C₃₋₈ cycloalkyl.

In some embodiments, the compound is selected from the group consisting of compounds 1 to 977 and pharmaceutically acceptable salts thereof.

In another aspect, the invention provides a pharmaceutical composition including the compound disclosed herein, or a pharmaceutically acceptable salt thereof, and a pharmaceutically acceptable excipient. In some embodiments, the composition is isotopically enriched in deuterium.

In yet another aspect, the invention provides a method of inhibiting Myt1 in a cell expressing Myt1, the method including contacting the cell with the compound disclosed herein.

In some embodiments, the cell is overexpressing CCNE1. In some embodiments, the cell is in a subject.

In still another aspect, the invention provides a method of treating a subject in need thereof including administering to the subject the compound disclosed herein, or a pharmaceutically acceptable salt thereof, or the pharmaceutical composition disclosed herein.

In some embodiments, the subject is suffering from, and is in need of a treatment for, a disease or condition having the symptom of cell hyperproliferation. In some embodiments, the disease or condition is a cancer. In some embodiments, the cancer is a cancer overexpressing CCNE1.

In still another aspect, the invention provides a method of treating a cancer in a subject, the method including administering to the subject in need thereof a therapeutically effective amount of a Myt1 inhibitor, where the cancer has been previously identified as a cancer overexpressing CCNE1.

In another aspect, the invention provides a method of treating a cancer in a subject, the method including administering to the subject in need thereof a therapeutically effective amount of a Myt1 inhibitor, where the cancer is a cancer overexpressing CCNE1.

In yet another aspect, the invention provides a method of inducing cell death in a cancer cell overexpressing CCNE1, the method including contacting the cell with an effective amount of a Myt1 inhibitor.

In some embodiments, the cell is in a subject. In some embodiments, the Myt1 inhibitor is the compound disclosed herein or a pharmaceutically acceptable salt thereof. In some embodiments, the cancer overexpressing CCNE1 is uterine cancer, ovarian cancer, breast cancer, stomach cancer, esophageal cancer, lung cancer, or endometrial cancer.

In still another aspect, the invention provides a method of treating a cancer in a subject, the method including administering to the subject in need thereof a therapeutically effective amount of a Myt1 inhibitor, where the cancer has been previously identified as a cancer having an inactivating mutation in the FBXW7 gene.

In another aspect, the invention provides a method of treating a cancer in a subject, the method including administering to the subject in need thereof a therapeutically effective amount of a Myt1 inhibitor, where the cancer has an inactivating mutation in the FBXW7 gene.

In yet another aspect, the invention provides a method of inducing cell death in an FBXW7-mutated cancer cell, the method including contacting the cell with an effective amount of a Myt1 inhibitor.

In some embodiments, the cell is in a subject. In some embodiments, the cancer is uterine cancer, colorectal cancer, breast cancer, lung cancer, or esophageal cancer. In some embodiments, the Myt1 inhibitor is the compound disclosed herein, or a pharmaceutically acceptable salt thereof.

### Abbreviations

Abbreviations and terms that are commonly used in the fields of organic chemistry, medicinal chemistry, pharmacology, and medicine and are well known to practitioners in these fields are used herein. Representative abbreviations and definitions are provided below:
Ac is acetyl [CH₃C(O)—];
ACN is acetonitrile;
Ac₂O is acetic anhydride;
AcOH is acetic acid;
APC is antigen-presenting cell;
Ar is aryl;
aq. is aqueous;
9-BBN is 9-borabicyclo[3.3.1]nonane;
BINAP is (2,2′-bis(diphenylphosphino)-1,1′-binaphthyl);
Bn is benzyl;
Boc is tert Butyloxycarbonyl;
n-BuLi is n-butyl lithium;
CDI is carbonyldiimidazole;
cmpd is compound;
conc. is concentrated;
DCM is dichloromethane;
DIAD is diisopropylazodicarboxylate;
DIBAL is diisobutylaluminum hydride;
DIPEA is diisoproplyethyl amine;
DMA is dimethylacetamide;
DMAP is 4-dimethylaminopyridine;
DME is dimethoxyethane;
DMF is N,N-dimethylformamide;
DMSO is dimethyl sulfoxide;
dppf is 1,1′-bis(diphenylphosphino)ferrocene;
dtbpf is 1,1′-Bis(di-tert-butylphosphino)ferrocene;
EDAC (or EDC) is 1-ethyl-3-[3-(dimethylamino)propyl]-carbodiimide HCl;
ESI is electrospray ionization mass spectrometry;
Et₂O is diethyl ether;
Et₃N is triethylamine;
Et is ethyl;
EtOAc is ethyl acetate;
EtOH is ethanol;
3-F-Ph is 3-fluorophenyl,
h is hour;
HATU is (1-[bis(dimethylamino)methylene]-1H-1,2,3-triazolo[4,5-b]pyridinium 3-oxide hexafluorophosphate;
HCl is hydrochloric acid;
Het is heteroaryl;
Hex is hexanes;
HOBt is 1-hydroxybenzotriazole;
HPLC is high performance liquid chromatography;
IPA is isopropanol;
IPAc is isopropyl acetate;
LCMS is HPLC with mass spectral detection;
LiHMDS is lithium bis(trimethylsilyl)amide;
LG is leaving group;
M is molar;
mCPBA is metachloroperbenzoic acid;
mmol is millimole;
Me is methyl;
MeCN is acetonitrile;
MeMgBr is methylmagnesium bromide;
MeMgCl is methylmagnesium chloride;
MeOH is methanol;
min is minute;
MOM is methoxymethyl;
Ms is methanesulfonyl;
MS is mass spectrometry;
MTBE is methyl tert-butyl ether;
MW is microwave;
N is normal;
NaHMDS is sodium bis(trimethylsilyl)amide;
NaOAc is sodium acetate;
NaOtBu is sodium tert-butoxide;
NBS is N-bromosuccinimide;
NCS is N-chlorosuccinimide;
NIS is N-iodosuccinimide;
NMO is N-methylmorpholine N-oxide;
NMP is N-methyl pyrrolidinone;
NMR is nuclear magnetic resonance spectroscopy;
PdCl₂(dppf) is [1,1′-Bis(diphenylphosphino)ferrocene]dichloropalladium(II);
PdCl₂(dppf)·CH₂Cl₂ is [1,1′-Bis(diphenylphosphino)ferrocene]dichloropalladium(II), complex with dichloromethane;
Pd₂(dba)₃ is tris(dibenzylideneacetone)dipalladium;
PdCl₂(PPh₃)₂ is dichlorobis-(triphenylphosphene) palladium;
Pd-PEPPSI™-SIPr is (1,3-Bis(2,6-diisopropylphenyl)imidazolidene) (3-chloropyridyl) palladium(II) dichloride;
PG Denotes a protecting group;
Ph is phenyl;
PhMe is toluene;
PIV-Cl is pivaloyl chloride, Trimethylacetyl chloride;
PPh₃ is triphenylphosphine;
PMB is para-methoxybenzyl;
Reagent alcohol is a mixture of 90% ethanol, 5% isopropanol and 5% methanol;
rt or RT is room temperature;
RBF is round-bottom flask;
RuPhos Pd G1 is chloro-(2-Dicyclohexylphosphino-2′,6′-diisopropoxy-1,1′-biphenyl)[2-(2-aminoethyl)phenyl]palladium(II);
sat. is saturated;
SEM is [2-(trimethylsilyl)ethoxy]methyl;
SFC is supercritical fluid chromatography;
S_{N}Ar is nucleophilic aromatic substitution;
TBAB is tetrabutyl ammonium bromide;
TBAF is tetrabutyl ammonium fluoride;
TBS is tert-butyldimethylsilyl;
tBu is tert-butyl;
Tf is trifluoromethanesulfonyl;
TFA is trifluoroacetic acid;
THF is tetrahydrofuran;
THP is tetrahydropyran;
TLC is thin layer chromatography;
TMAD is tetramethylazodicarboxamide;
TMS is trimethylsilyl;
TPAP is tetrapropylammonium perruthenate;
Ts is p-toluenesulfonyl;
UPLC is ultra-performance liquid chromatography;
Xantphos is 4,5-Bis(diphenylphosphino)-9,9-dimethylxanthene.

### Definitions

The term “aberrant,” as used herein, refers to different from normal. When used to describe activity, aberrant refers to activity that is greater or less than a normal control or the average of normal non-diseased control samples. Aberrant activity may refer to an amount of activity that results in a disease, where returning the aberrant activity to a normal or non-disease-associated amount (e.g. by administering a compound or using a method as described herein), results in reduction of the disease or one or more disease symptoms.

The term “acyl,” as used herein, represents a group —C(═O)—R, where R is alkyl, alkenyl, alkynyl, cycloalkyl, cycloalkenyl, cycloalkynyl, aryl, heteroaryl, or heterocyclyl. Acyl may be optionally substituted as described herein for each respective R group.

The term “adenocarcinoma,” as used herein, represents a malignancy of the arising from the glandular cells that line organs within an organism. Non-limiting examples of adenocarcinomas include non-small cell lung cancer, prostate cancer, pancreatic cancer, esophageal cancer, and colorectal cancer.

The term “alkanoyl,” as used herein, represents a hydrogen or an alkyl group that is attached to the parent molecular group through a carbonyl group and is exemplified by formyl (i.e., a carboxyaldehyde group), acetyl, propionyl, butyryl, and iso-butyryl. Unsubstituted alkanoyl groups contain from 1 to 7 carbons. The alkanoyl group may be unsubstituted of substituted (e.g., optionally substituted C₁₋₇ alkanoyl) as described herein for alkyl group. The ending “-oyl” may be added to another group defined herein, e.g., aryl, cycloalkyl, and heterocyclyl, to define “aryloyl,” “cycloalkanoyl,” and “(heterocyclyl)oyl.” These groups represent a carbonyl group substituted by aryl, cycloalkyl, or heterocyclyl, respectively. Each of “aryloyl,” “cycloalkanoyl,” and “(heterocyclyl)oyl” may be optionally substituted as defined for “aryl,” “cycloalkyl,” or “heterocyclyl,” respectively.

The term “alkenyl,” as used herein, represents acyclic monovalent straight or branched chain hydrocarbon groups of containing one, two, or three carbon-carbon double bonds. Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkenylene,” as used herein, refers to a divalent alkenyl group. An optionally substituted alkenylene is an alkenylene that is optionally substituted as described herein for alkenyl.

The term “alkoxy,” as used herein, represents a chemical substituent of formula —OR, where R is a C₁₋₆ alkyl group, unless otherwise specified. In some embodiments, the alkyl group can be further substituted as defined herein. The term “alkoxy” can be combined with other terms defined herein, e.g., aryl, cycloalkyl, or heterocyclyl, to define an “aryl alkoxy,” “cycloalkyl alkoxy,” and “(heterocyclyl)alkoxy” groups. These groups represent an alkoxy that is substituted by aryl, cycloalkyl, or heterocyclyl, respectively. Each of “aryl alkoxy,” “cycloalkyl alkoxy,” and “(heterocyclyl)alkoxy” may optionally substituted as defined herein for each individual portion.

The term “alkoxyalkyl,” as used herein, represents a chemical substituent of formula -L-O-R, where L is C₁₋₆ alkylene, and R is C₁₋₆ alkyl. An optionally substituted alkoxyalkyl is an alkoxyalkyl that is optionally substituted as described herein for alkyl.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: amino; alkoxy; aryl; aryloxy; azido; cycloalkyl; cycloalkoxy; cycloalkenyl; cycloalkynyl; halo; heterocyclyl; (heterocyclyl)oxy; heteroaryl; hydroxy; nitro; thiol; silyl; cyano; alkylsulfonyl; alkylsulfinyl; alkylsulfenyl; ═O; ═S; —C(O)R or —SO₂R, where R is amino; and =NR′, where R′ is H, alkyl, aryl, or heterocyclyl. Each of the substituents may itself be unsubstituted or, valency permitting, substituted with unsubstituted substituent(s) defined herein for each respective group.

The term “alkylene,” as used herein, refers to a divalent alkyl group. An optionally substituted alkylene is an alkylene that is optionally substituted as described herein for alkyl.

The term “alkylamino,” as used herein, refers to a group having the formula —N(R^{N1})₂ or —NHR^{N1}, in which R^{N1} is alkyl, as defined herein. The alkyl portion of alkylamino can be optionally substituted as defined for alkyl. Each optional substituent on the substituted alkylamino may itself be unsubstituted or, valency permitting, substituted with unsubstituted substituent(s) defined herein for each respective group.

The term “alkylsulfenyl,” as used herein, represents a group of formula —S-(alkyl). Alkylsulfenyl may be optionally substituted as defined for alkyl.

The term “alkylsulfinyl,” as used herein, represents a group of formula —S(O)-(alkyl). Alkylsulfinyl may be optionally substituted as defined for alkyl.

The term “alkylsulfonyl,” as used herein, represents a group of formula —S(O)₂-(alkyl). Alkylsulfonyl may be optionally substituted as defined for alkyl.

The term “alkynyl,” as used herein, represents monovalent straight or branched chain hydrocarbon groups of from two to six carbon atoms containing at least one carbon-carbon triple bond and is exemplified by ethynyl, 1-propynyl, and the like. The alkynyl groups may be unsubstituted or substituted (e.g., optionally substituted alkynyl) as defined for alkyl.

The term “alkynylene,” as used herein, refers to a divalent alkynyl group. An optionally substituted alkynylene is an alkynylene that is optionally substituted as described herein for alkynyl.

The term “amino,” as used herein, represents —N(R^{N1})₂, where, if amino is unsubstituted, both R^{N1} are H; or, if amino is substituted, each R^{N1} is independently H, —OH, —NO₂, —N(R^{N2})₂, —SO₂OR^{N2}, —SO₂R^{N2}, —SOR^{N2}, —C(O)OR^{N2}, an N-protecting group, alkyl, alkenyl, alkynyl, alkoxy, aryl, arylalkyl, aryloxy, cycloalkyl, cycloalkenyl, heteroalkyl, or heterocyclyl, provided that at least one R^{N1} is not H, and where each R^{N2} is independently H, alkyl, or aryl. Each of the substituents may itself be unsubstituted or substituted with unsubstituted substituent(s) defined herein for each respective group. In some embodiments, amino is unsubstituted amino (i.e., —NH₂) or substituted amino (e.g., —NHR^{N1}), where R^{N1} is independently —OH, SO₂OR^{N2}, SO₂R^{N2}, —SOR^{N2}, —COOR^{N2}, optionally substituted alkyl, or optionally substituted aryl, and each R^{N2} can be optionally substituted alkyl or optionally substituted aryl. In some embodiments, substituted amino may be alkylamino, in which the alkyl groups are optionally substituted as described herein for alkyl. In some embodiments, an amino group is —NHR^{N1}, in which R^{N1} is optionally substituted alkyl.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; —(CH₂)ₙ—C(O)OR^{A}; —C(O)R; and —SO₂R, where R is amino or alkyl, R^{A} is H or alkyl, and n is 0 or 1. Each of the substituents may itself be unsubstituted or substituted with unsubstituted substituent(s) defined herein for each respective group.

The term “aryl alkyl,” as used herein, represents an alkyl group substituted with an aryl group. The aryl and alkyl portions may be optionally substituted as the individual groups as described herein.

The term “arylene,” as used herein, refers to a divalent aryl group. An optionally substituted arylene is an arylene that is optionally substituted as described herein for aryl.

The term “aryloxy,” as used herein, represents a chemical substituent of formula —OR, where R is an aryl group, unless otherwise specified. In optionally substituted aryloxy, the aryl group is optionally substituted as described herein for aryl.

The term “azido,” as used herein, represents an —N₃ group.

The term “cancer,” as used herein, refers to all types of cancer, neoplasm or malignant tumors found in mammals (e.g., humans).

The term “carbocyclic,” as used herein, represents an optionally substituted C3-16 monocyclic, bicyclic, or tricyclic structure in which the rings, which may be aromatic or non-aromatic, are formed by carbon atoms. Carbocyclic structures include cycloalkyl, cycloalkenyl, cycloalkynyl, and certain aryl groups.

The term “carbonyl,” as used herein, represents a —C(O)— group.

The term “carcinoma,” as used herein, refers to a malignant new growth made up of epithelial cells tending to infiltrate the surrounding tissues and give rise to metastases.

The term “cyano,” as used herein, represents —CN group.

The terms “CCNE1” and “cyclin E1,” as used interchangeably herein, refer to G1/S specific cyclin E1 (Gene name: CCNE1). A cell overexpressing CCNE1 is one that exhibits a higher activity of CCNE1 than a cell normally expressing CCNE1. For example, a CCNE1-overexpressing cell is a cell that exhibits a copy number of at least 3 compared to a diploid normal cell with 2 copies. Thus, a cell exhibiting a copy number greater than 3 of CCNE1 is a cell overexpressing CCNE1. The CCNE1 overexpression may be measured by identifying the expression level of the gene product in a cell (e.g., CCNE1 mRNA transcript count or CCNE1 protein level).

The term “cycloalkenyl,” as used herein, refers to a non-aromatic carbocyclic group having at least one double bond in the ring and from three to ten carbons (e.g., a C₃₋₁₀ cycloalkenyl), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenyl alkyl,” as used herein, represents an alkyl group substituted with a cycloalkenyl group, each as defined herein. The cycloalkenyl and alkyl portions may be substituted as the individual groups defined herein.

The term “cycloalkenylene,” as used herein, represents a divalent cycloalkenyl group. An optionally substituted cycloalkenylene is a cycloalkenylene that is optionally substituted as described herein for cycloalkyl.

The term “cycloalkoxy,” as used herein, represents a chemical substituent of formula —OR, where R is cycloalkyl group, unless otherwise specified. In some embodiments, the cycloalkyl group can be further substituted as defined herein.

The term “cycloalkyl,” as used herein, refers to a cyclic alkyl group having from three to ten carbons (e.g., a C_{3-C10} cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; heteroaryl; hydroxy; nitro; thiol; silyl; cyano; ═O; ═S; —SO₂R, where R is optionally substituted amino; ═NR′, where R′ is H, alkyl, aryl, or heterocyclyl; and —CON(R^{A})₂, where each R^{A} is independently H or alkyl, or both R^{A}, together with the atom to which they are attached, combine to form heterocyclyl. Each of the substituents may itself be unsubstituted or substituted with unsubstituted substituent(s) defined herein for each respective group.

The term “cycloalkyl alkyl,” as used herein, represents an alkyl group substituted with a cycloalkyl group, each as defined herein. The cycloalkyl and alkyl portions may be optionally substituted as the individual groups described herein.

The term “cycloalkylene,” as used herein, represents a divalent cycloalkyl group. An optionally substituted cycloalkylene is a cycloalkylene that is optionally substituted as described herein for cycloalkyl.

The term “cycloalkynyl,” as used herein, refers to a monovalent carbocyclic group having one or two carbon-carbon triple bonds and having from eight to twelve carbons, unless otherwise specified. Cycloalkynyl may include one transannular bond or bridge. Non-limiting examples of cycloalkynyl include cyclooctynyl, cyclononynyl, cyclodecynyl, and cyclodecadiynyl. The cycloalkynyl group may be unsubstituted or substituted (e.g., optionally substituted cycloalkynyl) as defined for cycloalkyl.

“Disease” or “condition” refer to a state of being or health status of a patient or subject capable of being treated with the compounds or methods provided herein.

The term “FBXW7,” as used herein, refers to F-box/WD Repeat-Containing Protein 7 gene, transcript, or protein. An FBXW7-mutated gene, also described herein as an FBXW7 gene having an inactivating mutation, is one that fails to produce a functional FBXW7 protein or produces reduced quantities of FBXW7 protein in a cell.

The term “halo,” as used herein, represents a halogen selected from bromine, chlorine, iodine, and fluorine.

The term “heteroalkyl,” as used herein refers to an alkyl, alkenyl, or alkynyl group interrupted once by one or two heteroatoms; twice, each time, independently, by one or two heteroatoms; three times, each time, independently, by one or two heteroatoms; or four times, each time, independently, by one or two heteroatoms. Each heteroatom is, independently, O, N, or S. In some embodiments, the heteroatom is O or N. None of the heteroalkyl groups includes two contiguous oxygen or sulfur atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteroatom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N protecting group, alkyl, alkenyl, alkynyl, aryl, cycloalkyl, cycloalkenyl, cycloalkynyl, heterocyclyl, or cyano, where each R^{N2} is independently H, alkyl, cycloalkyl, cycloalkenyl, cycloalkynyl, aryl, or heterocyclyl, and each R^{N3} is independently alkyl, cycloalkyl, cycloalkenyl, cycloalkynyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. It is understood that carbon atoms are found at the termini of a heteroalkyl group.

The term “heteroaryl alkyl,” as used herein, represents an alkyl group substituted with a heteroaryl group, each as defined herein. The heteroaryl and alkyl portions may be optionally substituted as the individual groups described herein.

The term “heteroarylene,” as used herein, represents a divalent heteroaryl. An optionally substituted heteroarylene is a heteroarylene that is optionally substituted as described herein for heteroaryl.

The term “heteroaryloxy,” as used herein, refers to a structure —OR, in which R is heteroaryl. Heteroaryloxy can be optionally substituted as defined for heterocyclyl.

The term “heterocyclyl,” as used herein, represents a monocyclic, bicyclic, tricyclic, or tetracyclic ring system having fused, bridging, and/or spiro 3-, 4-, 5-, 6-, 7-, or 8-membered rings, unless otherwise specified, containing one, two, three, or four heteroatoms independently selected from the group consisting of nitrogen, oxygen, and sulfur. In some embodiments, “heterocyclyl” is a monocyclic, bicyclic, tricyclic, or tetracyclic ring system having fused or bridging 5-, 6-, 7-, or 8-membered rings, unless otherwise specified, containing one, two, three, or four heteroatoms independently selected from the group consisting of nitrogen, oxygen, and sulfur. Heterocyclyl can be aromatic or non-aromatic. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups include from 1 to 16 carbon atoms unless otherwise specified. Certain heterocyclyl groups may include up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, etc. If the heterocyclic ring system has at least one aromatic resonance structure or at least one aromatic tautomer, such structure is an aromatic heterocyclyl (i.e., heteroaryl). Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; cyano; —C(O)R or —SO₂R, where R is amino or alkyl; ═O; ═S; =NR′, where R′ is H, alkyl, aryl, or heterocyclyl. Each of the substituents may itself be unsubstituted or substituted with unsubstituted substituent(s) defined herein for each respective group.

The term “heterocyclyl alkyl,” as used herein, represents an alkyl group substituted with a heterocyclyl group, each as defined herein. The heterocyclyl and alkyl portions may be optionally substituted as the individual groups described herein.

The term “heterocyclylene,” as used herein, represents a divalent heterocyclyl. An optionally substituted heterocyclylene is a heterocyclylene that is optionally substituted as described herein for heterocyclyl.

The term “(heterocyclyl)oxy,” as used herein, represents a chemical substituent of formula —OR, where R is a heterocyclyl group, unless otherwise specified. (Heterocyclyl)oxy can be optionally substituted in a manner described for heterocyclyl.

The terms “hydroxyl” and “hydroxy,” as used interchangeably herein, represent an —OH group.

The term “isotopically enriched,” as used herein, refers to the pharmaceutically active agent with the isotopic content for one isotope at a predetermined position within a molecule that is at least 100 times greater than the natural abundance of this isotope. For example, a composition that is isotopically enriched for deuterium includes an active agent with at least one hydrogen atom position having at least 100 times greater abundance of deuterium than the natural abundance of deuterium. Preferably, an isotopic enrichment for deuterium is at least 1000 times greater than the natural abundance of deuterium. More preferably, an isotopic enrichment for deuterium is at least 4000 times greater (e.g., at least 4750 times greater, e.g., up to 5000 times greater) than the natural abundance of deuterium.

The term “leukemia,” as used herein, refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic).

The term “lymphoma,” as used herein, refers to a cancer arising from cells of immune origin.

The term “melanoma,” as used herein, is taken to mean a tumor arising from the melanocytic system of the skin and other organs.

The term “Myt1,” as used herein, refers to membrane-associated tyrosine and threonine-specific cdc2-inhibitory kinase (Myt1) (Gene name PKMYT1).

The term “Myt1 inhibitor,” as used herein, represents a compound that upon contacting the enzyme Myt1, whether in vitro, in cell culture, or in an animal, reduces the activity of Myt1, such that the measured Myt1 IC₅₀ is 10 μM or less (e.g., 5 μM or less or 1 μM or less). For certain Myt1 inhibitors, the Myt1 IC50 may be 100 nM or less (e.g., 10 nM or less, or 3 nM or less) and could be as low as 100 μM or 10 μM. Preferably, the Myt1 IC50 is 1 nM to 1 μM (e.g., 1 nM to 750 nM, 1 nM to 500 nM, or 1 nM to 250 nM). Even more preferably, the Myt1 IC₅₀ is less than 20 nm (e.g., 1 nM to 20 nM).

The term “nitro,” as used herein, represents an —NO₂ group.

The term “oxo,” as used herein, represents a divalent oxygen atom (e.g., the structure of oxo may be shown as ═O).

The term “Ph,” as used herein, represents phenyl.

The term “pharmaceutical composition,” as used herein, represents a composition containing a compound described herein, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable excipient” or “pharmaceutically acceptable carrier,” as used interchangeably herein, refers to any ingredient other than the compounds described herein (e.g., a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “pre-malignant” or “pre-cancerous,” as used herein, refers to a condition that is not malignant but is poised to become malignant.

The term “protecting group,” as used herein, represents a group intended to protect a hydroxy, an amino, or a carbonyl from participating in one or more undesirable reactions during chemical synthesis. The term “O-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino, amido, heterocyclic N—H, or hydrazine) group from participating in one or more undesirable reactions during chemical synthesis. Commonly used O- and N-protecting groups are disclosed in Greene, “Protective Groups in Organic Synthesis,” 3rd Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, a-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p methoxybenzyloxycarbonyl, p-nitrobenzyloxycarbonyl, 2-nitrobenzyloxycarbonyl, p bromobenzyloxycarbonyl, 3,4-dimethoxybenzyloxycarbonyl, 3,5 dimethoxybenzyl oxycarbonyl, 2,4-dimethoxybenzyloxycarbonyl, 4 methoxybenzyloxycarbonyl, 2-nitro-4,5-dimethoxybenzyloxycarbonyl, 3,4,5 trimethoxybenzyloxycarbonyl, 1-(p-biphenylyl)-1-methylethoxycarbonyl, α,α-dimethyl-3,5 dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, p-methoxybenzyl, 2,4-dimethoxybenzyl, triphenylmethyl, benzyloxymethyl, and the like, silylalkylacetal groups such as [2-(trimethylsilyl)ethoxy]methyl and silyl groups such as trimethylsilyl, and the like. Useful N-protecting groups are formyl, acetyl, benzoyl, pivaloyl, t-butylacetyl, alanyl, phenylsulfonyl, benzyl, dimethoxybenzyl, [2-(trimethylsilyl)ethoxy]methyl (SEM), tetrahydropyranyl (THP), t-butyloxycarbonyl (Boc), and benzyloxycarbonyl (Cbz).

The term “tautomer” refers to structural isomers that readily interconvert, often by relocation of a proton. Tautomers are distinct chemical species that can be identified by differing spectroscopic characteristics, but generally cannot be isolated individually. Non-limiting examples of tautomers include ketone—enol, enamine—imine, amide—imidic acid, nitroso—oxime, ketene—ynol, and amino acid—ammonium carboxylate.

The term “sarcoma” generally refers to a tumor which is made up of a substance like the embryonic connective tissue and is generally composed of closely packed cells embedded in a fibrillar or homogeneous substance.

The term “subject,” as used herein, represents a human or non-human animal (e.g., a mammal) that is suffering from, or is at risk of, disease or condition, as determined by a qualified professional (e.g., a doctor or a nurse practitioner) with or without known in the art laboratory test(s) of sample(s) from the subject. Preferably, the subject is a human. Non-limiting examples of diseases and conditions include diseases having the symptom of cell hyperproliferation, e.g., a cancer. “Treatment” and “treating,” as used herein, refer to the medical management of a subject with the intent to improve, ameliorate, stabilize, prevent or cure a disease or condition. This term includes active treatment (treatment directed to improve the disease or condition); causal treatment (treatment directed to the cause of the associated disease or condition); palliative treatment (treatment designed for the relief of symptoms of the disease or condition); preventative treatment (treatment directed to minimizing or partially or completely inhibiting the development of the associated disease or condition); and supportive treatment (treatment employed to supplement another therapy).

### BRIEF DESCRIPTION OF THE DRAWINGS

FIG. 1A is a bar graph showing the CCNE1 amplification/overexpression across tumors sequenced from TCGA PanCancer Atlas.

FIG. 1B is a scatter plot showing the CCNE1 gene expression data from TCGA PanCancer Atlas.

FIG. 2A is a bar graph showing the FBXW7 mutations across tumors sequenced from TCGA PanCancer Atlas.

FIG. 2B is a lollipop graph showing the frequency of FBXW7 mutations across the gene. This graph highlights three common arginine hotspot mutations (R465, R479, and R505) within the third and fourth WD40 repeats that disrupt recognition of the Cyclin E1 substrate and are classified as deleterious.

FIG. 3A is a bar graph showing the results of a proliferation assay using RPE1-hTERT Cas9 TP53−/− and CCNE1-overexpressing clones treated with different doses of compound A.

FIG. 3B is a series of images depicting the results of a clonogenic survival assay using RPE1-hTERT Cas9 TP53−/− and CCNE1-overexpressing clones transduced with PKMYT1 sgRNAs. Infected cells were plated at low density to measure their ability to form colonies of >50 cells. After 10 days of growth, the colonies were stained, imaged, and quantified. The results are normalized to the survival of RPE1-hTERT Cas9 TP53−/− parental and CCNE1-overexpressing clones transduced with a non-targeting LacZ control sgRNA.

FIG. 3C is a line graph showing the results of a proliferation assay using RPE1-hTERT Cas9 TP54−/− and CCNE1-overexpressing clones treated with different doses of compound A.

FIG. 4A is a bar graph showing the results of a clonogenic survival assay using FT282-hTERT TP53^{R175H} and CCNE1-overexpressing cells transduced with PKMYT1 sgRNAs. Infected cells were plated at low density to measure their ability to form colonies of >50 cells. After 10 days of growth, the colonies were stained, imaged, and quantified. The results are normalized to the survival of FT282-hTERT TP53^{R175H} and CCNE1-overexpressing cells transduced with an AAVS1 control sgRNA.

FIG. 4B is a series of images showing of stained colonies described in FIG. 4A.

FIG. 4C is a line graph showing the results of a proliferation assay using FT282-hTERT TP53^{R175H} and CCNE1-overexpressing clones treated with different doses of compound A.

FIGS. 5A, 5B, and 5C show the results of clonogenic survival assays for stable RPE1-hTERT Cas9 TP53−/− parental and CCNE1-overexpressing clones expressing either a wild type or catalytic-dead FLAG-tagged PKMYT1 sgRNA-resistant ORF. These stable cell lines were transduced with either a LacZ non-targeting sgRNA or PKMYT1 sgRNA #4 and plated at low density to measure their ability to form colonies of >50 cells. After 10 days of growth, the colonies were stained, imaged, and quantified. The results are normalized to the survival of RPE1-hTERT Cas9 TP53−/− CCNE1-overexpressing clones transduced with a non-targeting LacZ control sgRNA and represented as a bar graph in FIG. 5C. Both clones 2 and 21 behave similarly in this study.

FIG. 6 is a chart showing the results of proliferation assays for a panel of CCNE1 wild type and CCNE1-amplified/overexpressing cancer cell lines treated with different doses of compound B. The IC₅₀ values are plotted for each cell line and demonstrate that CCNE1-overexpressing cell lines show enhanced sensitivity to a Myt1 inhibitor compared to CCNE1 WT cell lines.

FIG. 7 is a chart showing the results of proliferation assays for a panel of FBXW7 wild type and FBXW7-mutated cancer cell lines treated with different doses of compound C. The IC50 values are plotted for each cell line and demonstrate that FBXW7-mutated cell lines show enhanced sensitivity to a Myt1 inhibitor compared to FBXW7 WT cell lines.

### DETAILED DESCRIPTION

In general, the invention provides compounds, pharmaceutical compositions containing the same, methods of preparing the compounds, and methods of use. Compounds of the invention may be Myt1 inhibitors. These compounds may be used to inhibit Myt1 in a cell, e.g., a cell in a subject (e.g., a cell overexpressing CCNE1 or having an inactivating mutation in the FBXW7 gene). The subject may be in need of a treatment for a disease or condition, e.g., a disease or condition having a symptom of cell hyperproliferation, e.g., a cancer. The Myt1 inhibitory activity of the compounds disclosed herein is useful for treating a subject in need of a treatment for cancer.

Myt1 is a cell cycle regulating kinase localized predominantly in the endoplasmic reticulum and golgi complex. It is part of the Wee family of kinases that includes Wee1 and Wee1b. It is involved in the negative regulation of the CDK1-Cyclin B complex which promotes the progression of cells from G2-phase into the mitotic phase (M-phase) of the cell cycle. During DNA damage, Myt1 drives the phosphorylation on CDK1 (both Tyr15 and Thr14 of CDK1) which maintains the kinase complex in an inactive state in G2 as part of the G2 checkpoint response along with Wee1 (which mediates only Tyr15 phosphorylation) and prevents entry into mitosis until the damage has been repaired. Additionally, it has been proposed that Myt1 directly interacts with CDK1 complexes in the cytoplasm and prevents their nuclear translocation thus inhibiting cell cycle progression.

Myt1 has been implicated as a potentially important cancer target as it is essential in many cancer cells. Overexpression of Myt1 has been observed in various cancers including hepatocellular carcinoma as well as clear-cell renal-cell carcinoma. Myt1 downregulation has a minor role in unperturbed cells but has a more prominent role in cells exposed to DNA damage. Additionally, cells that exhibit high levels of replication stress in addition to defective G1 checkpoint regulation may be particularly sensitive to loss of Myt1 function, as these cells will be prone to entering mitosis prematurely with compromised genomic material leading to mitotic catastrophe.

Inhibitors of Myt1, a regulator of G2-M transition, may be particularly useful in the treatment of tumors harboring CCNE1-amplification or FBXW7 loss-of-function mutations using a synthetic lethal therapeutic strategy.

Cyclin E1 (encoded by the CCNE1 gene) is involved in the G1 to S phase cell cycle transition. In late G1 phase of the cell cycle, it complexes with cyclin-dependent kinase 2 (CDK2) to promote E2F transcription factor activation and entry into S-phase. Cyclin E1 levels are tightly regulated during normal cell cycles, accumulating at the G1/S transition and being completely degraded by the end of S phase. The cell cycle-dependent proteasomal degradation of Cyclin E1 is mediated by the SCF^{FBW7} ubiquitin ligase complex. Once activated in late G1, the Cyclin E1/CDK2 complex promotes the transition into S phase through phosphorylation and inactivation of RB1 and subsequent release of E2F transcription factors. S phase is promoted by E2F-mediated transcription of numerous genes involved in DNA replication including the pre-replication complex subunits ORC1, CDC6, CDT1, and the MCM helicase factors.

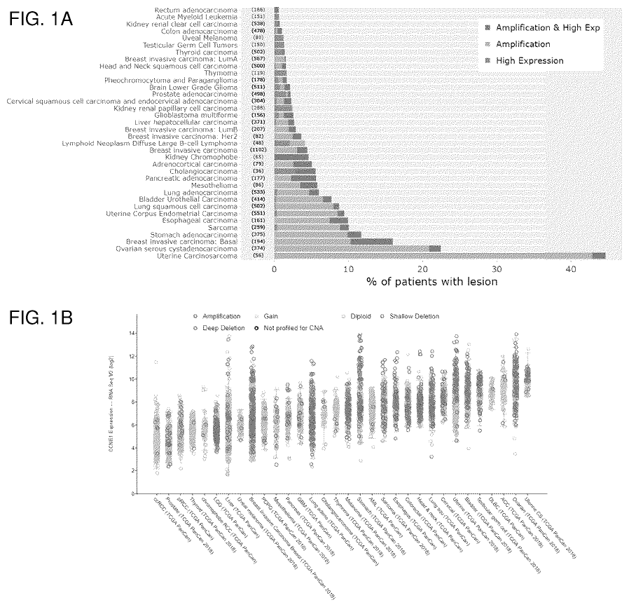

CCNE1 is frequently amplified and/or over-expressed in human cancers (FIG. 1). CCNE1 amplification has been reported in several cancer types including endometrial, ovarian, breast and gastric, ranging in frequency from 5-40%. Importantly, numerous studies have confirmed Cyclin E1 as a driver of tumorigenesis in these indications and CCNE1 amplification is observed in the more aggressive subtypes including uterine carcinosarcoma (UCS; ˜40%), uterine serous carcinoma (USC; ˜25%), high-grade serous ovarian carcinoma (HGSOC; ˜25%), and triple-negative breast cancer (TNBC; ˜8%). Patients with evidence of Cyclin E1 over-expression in tumor biopsies by immunohistochemistry and/or genomic copy number analysis have a lower overall survival compared to patients with normal Cyclin E1 levels. HGSOC patients with Cyclin E1 over-expression have a lower response rate to cisplatin, the current standard of care.

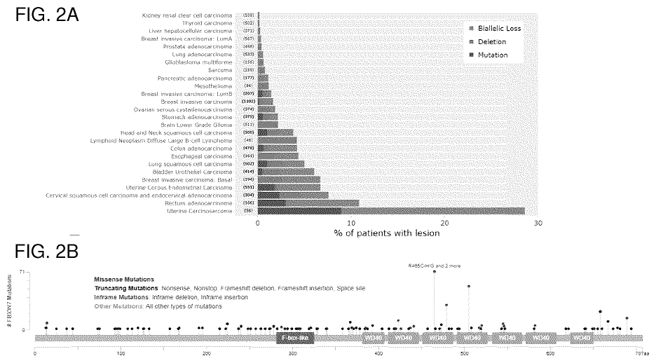

Defective cell cycle-regulated proteolysis of Cyclin E1 by the SCF^{FBW7} ubiquitin ligase complex is another mechanism of CCNE1 over-expression observed in tumors. The F-box protein gene, FBXW7, is frequently mutated in several cancer types including endometrial, colorectal, and gastric, ranging in frequency from 5-35% (FIG. 2). Like CCNE1, FBXW7 driver mutations are observed in the more aggressive subtypes of endometrial cancer including UCS (˜35%) and USC (˜25%). FBXW7 has a diverse spectrum of loss-of-function mutations in cancer including truncating mutations peppered across the gene and missense mutations within the Cyclin E1 recognizing WD40 repeats. FBW7 functions as a homodimer within the SCF complex and many deleterious missense mutations within the WD40 repeats are mostly heterozygous and dominant negative. Remarkably, several recurring hotspot missense mutations are found in the WD40 repeats including R465, R479, and R505—all of which disrupt Cyclin E1 binding and ubiquitylation.

Cyclin E1 over-expression and/or FBXW7 loss-of-function is thought to drive tumorigenesis by inducing genome instability (e.g., increased origin firing, defective nucleotide pools, transcription-replication conflicts, and/or fork instability). Over-expression of Cyclin E1 has been shown to induce replication stress characterized by slowed or stalled replication forks and loss-of-heterozygosity at fragile sites. The primary mechanism by which Cyclin E1 over-expression causes replication stress is increased origin firing in early S-phase followed by depletion of replication factors including nucleotide pools. The decrease in overall replication proteins and nucleotides decreases fork progression and causes stalling and subsequent collapse or reversal.

The compound of the invention may be, e.g., a compound of formula (I):

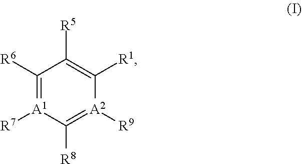

or a pharmaceutically acceptable salt thereof,
where
R¹ is:

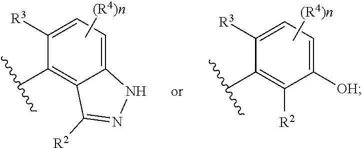

n is 0, 1, or 2;
each of R² and R³ is independently hydrogen, halogen, optionally substituted C₃₋₄ cycloalkyl or optionally substituted C₁₋₆ alkyl;
each R⁴ is independently halogen;
R⁵ is hydrogen, halogen, hydroxyl, optionally substituted C₁₋₆ alkyl, optionally substituted C₁₋₆ alkoxy, or —N(R^{5A})₂;
each R^{5A} is independently hydrogen, optionally substituted C₁₋₆ alkyl or optionally substituted C₃₋₈ cycloalkyl;
R⁶ is —C(O)NH(R^{6A}), —SO₂R^{6B}, or —(O)R^{6C};
R^{6A} is hydrogen, optionally substituted C₁₋₆ alkyl, optionally substituted C₃₋₈ cycloalkyl, optionally substituted C₂₋₉ heterocyclyl, optionally substituted C₆₋₁₀ aryl, or optionally substituted C₁₋₉ heteroaryl;
R^{6B} is optionally substituted C₁₋₆ alkyl, optionally substituted C₃₋₈ cycloalkyl, optionally substituted C₆₋₁₀ aryl, or —NH(R^{6A});
R^{6C} is optionally substituted C₁₋₆ alkyl;
each of A¹ and A² is independently N or C;
   R⁷ is absent, hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, or —OR¹⁰; R⁸ is hydrogen, halogen, optionally substituted C₁₋₆ alkyl, optionally substituted C₂₋₆ alkenyl, optionally substituted C₂₋₃ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR¹⁰, —N(R¹¹)₂, or -L-R^{3A}; or R⁷ and R⁸ combine with the atoms to which they are attached to form an optionally substituted C₃₋₈ cycloalkenyl, optionally substituted C₂₋₉ heterocyclyl, optionally substituted C₆₋₁₀ aryl, or optionally substituted C₂₋₁₂ heteroaryl; and R⁹ is absent, hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, or —OR¹⁰;
   or
   R⁸ is hydrogen, halogen, optionally substituted C₁₋₆ alkyl, optionally substituted C₂₋₆ alkenyl, optionally substituted C₂₋₃ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR¹⁰, —N(R¹¹)₂, or -L-R^{3A}; and R⁹ is absent, hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, or —OR¹⁰; or R⁸ and R⁹ combine with the atoms to which they are attached to form an optionally substituted C₃₋₈ cycloalkenyl, optionally substituted C₂₋₉ heterocyclyl, optionally substituted C₆₋₁₀ aryl, or optionally substituted C₁₋₁₂ heteroaryl; and R⁷ is absent, hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, or —OR¹⁰;
L is optionally substituted C₂₋₉ heterocyclylene, optionally substituted C₂₋₉ heteroarylene, optionally substituted C₆₋₁₀ arylene, or optionally substituted C₃₋₈ cycloalkylene;
R^{8A} is hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR¹⁰, or —N(R¹¹)₂;
R¹⁰ is hydrogen, optionally substituted C₁₋₆ alkyl, optionally substituted C₁-s heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R¹¹ is independently hydrogen, halogen, optionally substituted C₁₋₆ alkyl, optionally substituted acyl, optionally substituted C₁₋₃ heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl, —SO₂R^{11A} or two R¹¹ groups combine to form an optionally substituted C₂₋₉ heterocyclyl; and
each R^{11A} is independently hydrogen, optionally substituted C₁₋₆ alkyl, optionally substituted C₂₋₆ alkenyl, optionally substituted C₂₋₆ alkynyl, optionally substituted C₁₋₃ heteroalkyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl.

The compound of the invention may be, e.g., a compound listed in Table 1 below or a pharmaceutically acceptable salt thereof.

TABLE 1

| | |
|---|---|
| 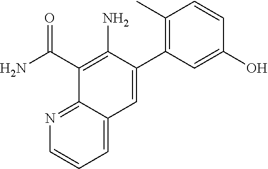 [SMILES: Cc1ccc(O)cc1-c1cc2cccnc2c(C(N)=O)c1N] | 1 |
| 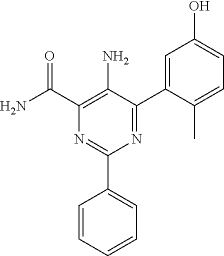 [SMILES: Cc1ccc(O)cc1-c1nc(-c2ccccc2)nc(C(N)=O)c1N] | 2 |
| 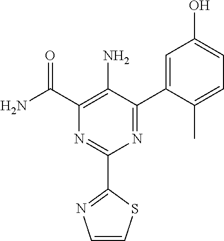 [SMILES: Cc1ccc(O)cc1-c1nc(-c2nccs2)nc(C(N)=O)c1N] | 3 |
| 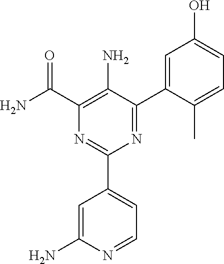 [SMILES: Cc1ccc(O)cc1-c1nc(-c2ccnc(N)c2)nc(C(N)=O)c1N] | 4 |
| 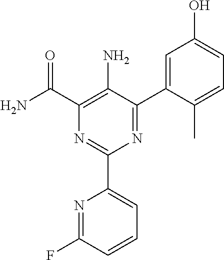 [SMILES: Cc1ccc(O)cc1-c1nc(-c2cccc(F)n2)nc(C(N)=O)c1N] | 5 |
| 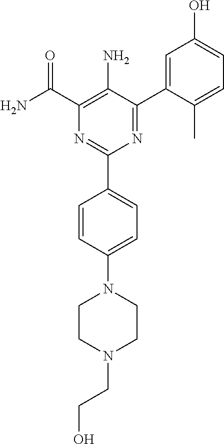 [SMILES: Cc1ccc(O)cc1-c1nc(-c2ccc(N3CCN(CCO)CC3)cc2)nc(C(N)=O)c1N] | 6 |
| 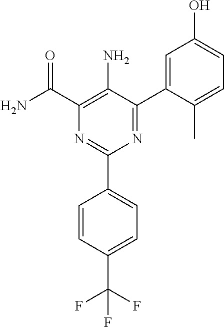 [SMILES: Cc1ccc(O)cc1-c1nc(-c2ccc(C(F)(F)F)cc2)nc(C(N)=O)c1N] | 7 |
| 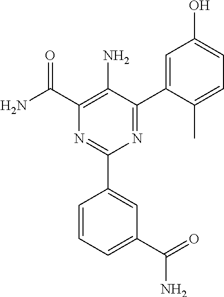 [SMILES: Cc1ccc(O)cc1-c1nc(-c2cccc(C(N)=O)c2)nc(C(N)=O)c1N] | 8 |
| 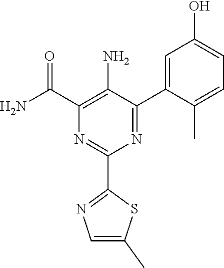 [SMILES: Cc1cnc(-c2nc(C(N)=O)c(N)c(-c3cc(O)ccc3C)n2)s1] | 9 |
| 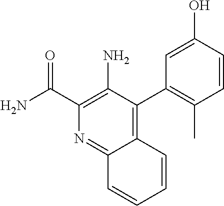 [SMILES: Cc1ccc(O)cc1-c1c(N)c(C(N)=O)nc2ccccc12] | 10 |
| 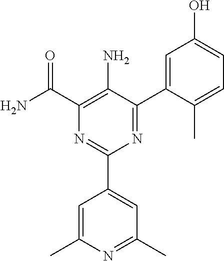 [SMILES: Cc1cc(-c2nc(C(N)=O)c(N)c(-c3cc(O)ccc3C)n2)cc(C)n1] | 11 |
| 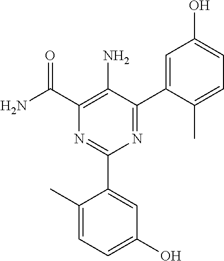 [SMILES: Cc1ccc(O)cc1-c1nc(C(N)=O)c(N)c(-c2cc(O)ccc2C)n1] | 12 |
| 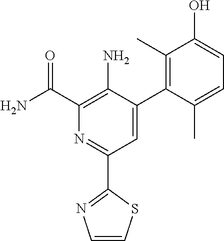 [SMILES: Cc1ccc(O)c(C)c1-c1cc(-c2nccs2)nc(C(N)=O)c1N] | 13 |
| 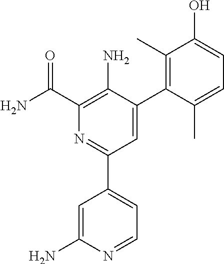 [SMILES: Cc1ccc(O)c(C)c1-c1cc(-c2ccnc(N)c2)nc(C(N)=O)c1N] | 14 |
| 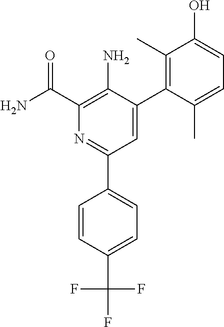 [SMILES: Cc1ccc(O)c(C)c1-c1cc(-c2ccc(C(F)(F)F)cc2)nc(C(N)=O)c1N] | 15 |
| 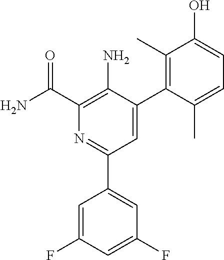 [SMILES: Cc1ccc(O)c(C)c1-c1cc(-c2cc(F)cc(F)c2)nc(C(N)=O)c1N] | 16 |
| 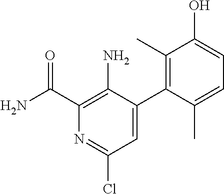 [SMILES: Cc1ccc(O)c(C)c1-c1cc(Cl)nc(C(N)=O)c1N] | 17 |
| 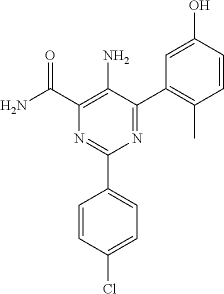 [SMILES: Cc1ccc(O)cc1-c1nc(-c2ccc(Cl)cc2)nc(C(N)=O)c1N] | 18 |
| 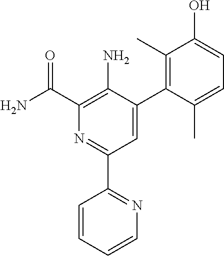 [SMILES: Cc1ccc(O)c(C)c1-c1cc(-c2ccccn2)nc(C(N)=O)c1N] | 19 |
| 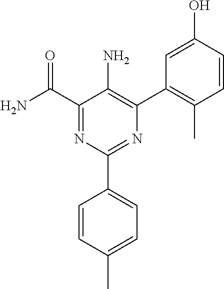 [SMILES: Cc1ccc(-c2nc(C(N)=O)c(N)c(-c3cc(O)ccc3C)n2)cc1] | 20 |
| 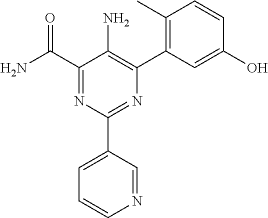 [SMILES: Cc1ccc(O)cc1-c1nc(-c2cccnc2)nc(C(N)=O)c1N] | 21 |
| 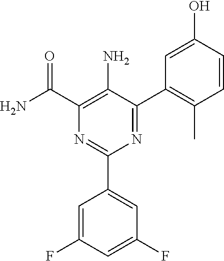 [SMILES: Cc1ccc(O)cc1-c1nc(-c2cc(F)cc(F)c2)nc(C(N)=O)c1N] | 22 |
| 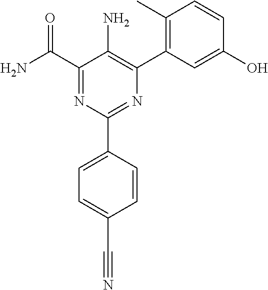 [SMILES: Cc1ccc(O)cc1-c1nc(-c2ccc(C#N)cc2)nc(C(N)=O)c1N] | 23 |
| 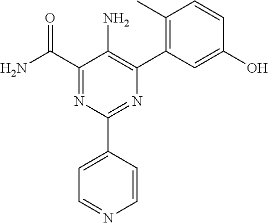 [SMILES: Cc1ccc(O)cc1-c1nc(-c2ccncc2)nc(C(N)=O)c1N] | 24 |
| 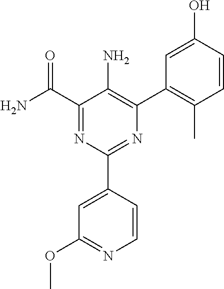 [SMILES: COc1cc(-c2nc(C(N)=O)c(N)c(-c3cc(O)ccc3C)n2)ccn1] | 25 |
| 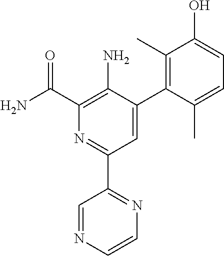 [SMILES: Cc1ccc(O)c(C)c1-c1cc(-c2cnccn2)nc(C(N)=O)c1N] | 26 |
| 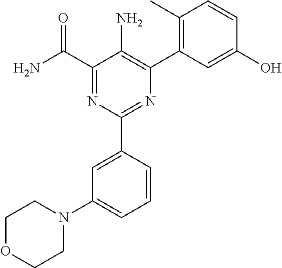 [SMILES: Cc1ccc(O)cc1-c1nc(-c2cccc(N3CCOCC3)c2)nc(C(N)=O)c1N] | 27 |
| 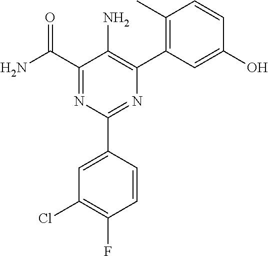 [SMILES: Cc1ccc(O)cc1-c1nc(-c2ccc(F)c(Cl)c2)nc(C(N)=O)c1N] | 28 |
| 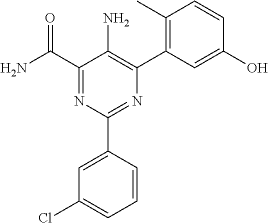 [SMILES: Cc1ccc(O)cc1-c1nc(-c2cccc(Cl)c2)nc(C(N)=O)c1N] | 29 |
| 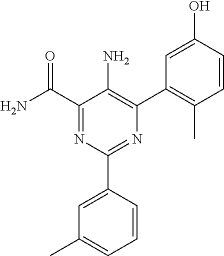 [SMILES: Cc1cccc(-c2nc(C(N)=O)c(N)c(-c3cc(O)ccc3C)n2)c1] | 30 |
| 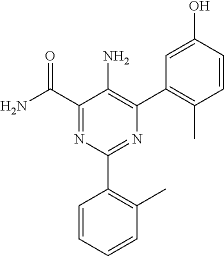 [SMILES: Cc1ccccc1-c1nc(C(N)=O)c(N)c(-c2cc(O)ccc2C)n1] | 31 |
| 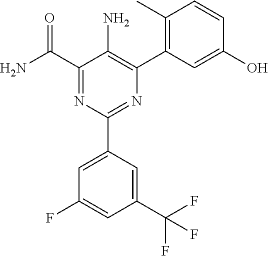 [SMILES: Cc1ccc(O)cc1-c1nc(-c2cc(F)cc(C(F)(F)F)c2)nc(C(N)=O)c1N] | 32 |
| 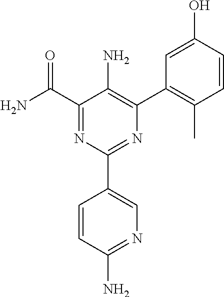 [SMILES: Cc1ccc(O)cc1-c1nc(-c2ccc(N)nc2)nc(C(N)=O)c1N] | 33 |
| 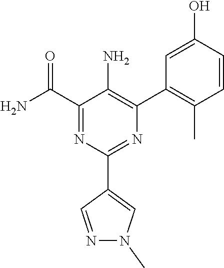 [SMILES: Cc1ccc(O)cc1-c1nc(-c2cnn(C)c2)nc(C(N)=O)c1N] | 34 |
| 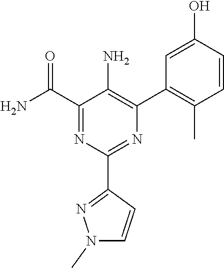 [SMILES: Cc1ccc(O)cc1-c1nc(-c2ccn(C)n2)nc(C(N)=O)c1N] | 35 |
| 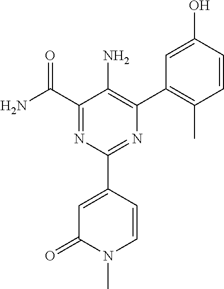 [SMILES: Cc1ccc(O)cc1-c1nc(-c2ccn(C)c(=O)c2)nc(C(N)=O)c1N] | 36 |
| 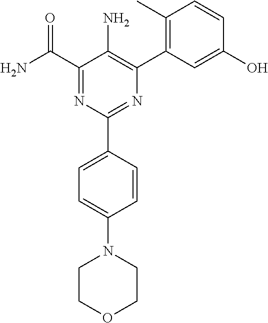 [SMILES: Cc1ccc(O)cc1-c1nc(-c2ccc(N3CCOCC3)cc2)nc(C(N)=O)c1N] | 37 |
| 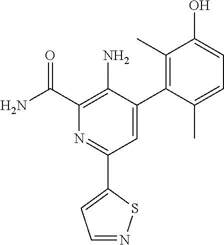 [SMILES: Cc1ccc(O)c(C)c1-c1cc(-c2ccns2)nc(C(N)=O)c1N] | 38 |
| 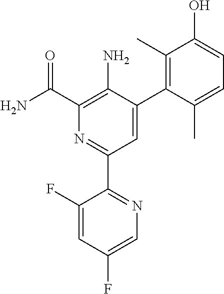 [SMILES: Cc1ccc(O)c(C)c1-c1cc(-c2ncc(F)cc2F)nc(C(N)=O)c1N] | 39 |
| 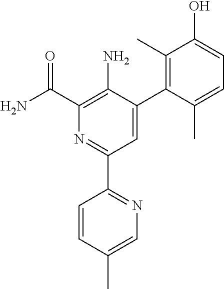 [SMILES: Cc1ccc(-c2cc(-c3c(C)ccc(O)c3C)c(N)c(C(N)=O)n2)nc1] | 40 |
| 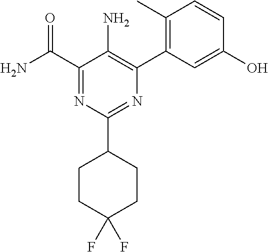 [SMILES: Cc1ccc(O)cc1-c1nc(C2CCC(F)(F)CC2)nc(C(N)=O)c1N] | 41 |
| 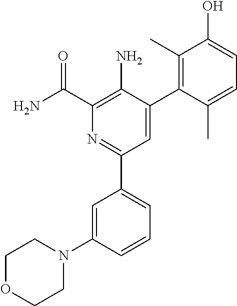 [SMILES: Cc1ccc(O)c(C)c1-c1cc(-c2cccc(N3CCOCC3)c2)nc(C(N)=O)c1N] | 42 |
| 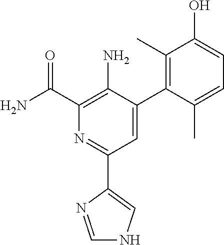 [SMILES: Cc1ccc(O)c(C)c1-c1cc(-c2c[nH]cn2)nc(C(N)=O)c1N] | 43 |
| 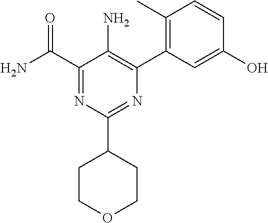 [SMILES: Cc1ccc(O)cc1-c1nc(C2CCOCC2)nc(C(N)=O)c1N] | 44 |
| 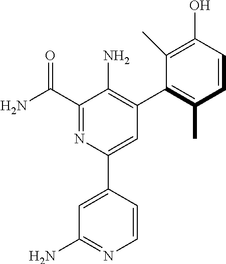 | 45 |
| 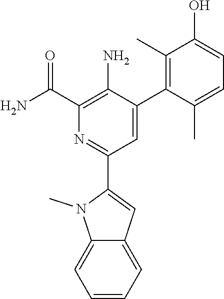 [SMILES: Cc1ccc(O)c(C)c1-c1cc(-c2cc3ccccc3n2C)nc(C(N)=O)c1N] | 46 |
| 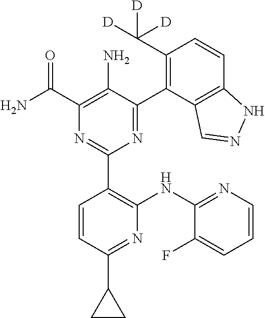 [SMILES: [2H]C([2H])([2H])c1ccc2[nH]ncc2c1-c1nc(-c2ccc(C3CC3)nc2Nc2ncccc2F)nc(C(N)=O)c1N] | 47 |
| 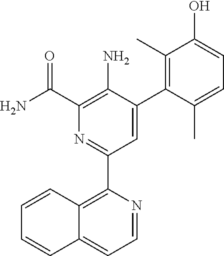 [SMILES: Cc1ccc(O)c(C)c1-c1cc(-c2nccc3ccccc23)nc(C(N)=O)c1N] | 48 |
| 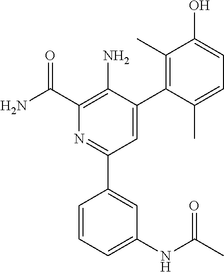 [SMILES: CC(=O)Nc1cccc(-c2cc(-c3c(C)ccc(O)c3C)c(N)c(C(N)=O)n2)c1] | 49 |
| 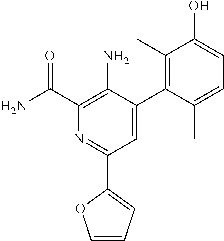 [SMILES: Cc1ccc(O)c(C)c1-c1cc(-c2ccco2)nc(C(N)=O)c1N] | 50 |
| 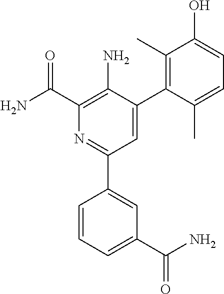 [SMILES: Cc1ccc(O)c(C)c1-c1cc(-c2cccc(C(N)=O)c2)nc(C(N)=O)c1N] | 51 |
| 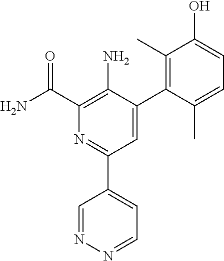 [SMILES: Cc1ccc(O)c(C)c1-c1cc(-c2ccnnc2)nc(C(N)=O)c1N] | 52 |
| 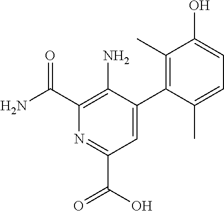 [SMILES: Cc1ccc(O)c(C)c1-c1cc(C(=O)O)nc(C(N)=O)c1N] | 53 |
| 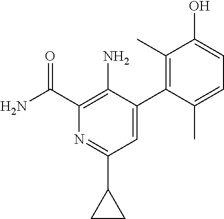 [SMILES: Cc1ccc(O)c(C)c1-c1cc(C2CC2)nc(C(N)=O)c1N] | 54 |
| 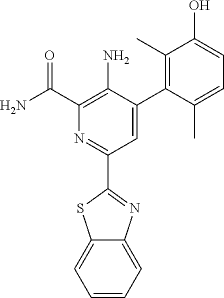 [SMILES: Cc1ccc(O)c(C)c1-c1cc(-c2nc3ccccc3s2)nc(C(N)=O)c1N] | 55 |
| 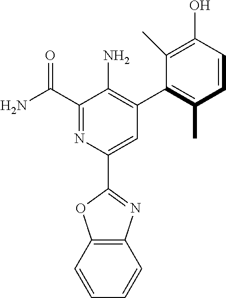 | 56 |
| 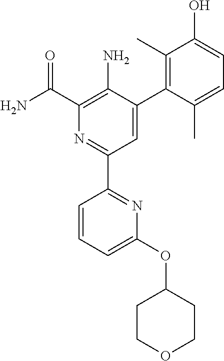 [SMILES: Cc1ccc(O)c(C)c1-c1cc(-c2cccc(OC3CCOCC3)n2)nc(C(N)=O)c1N] | 57 |
| 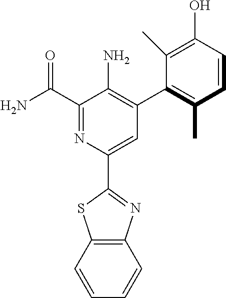 | 58 |
| 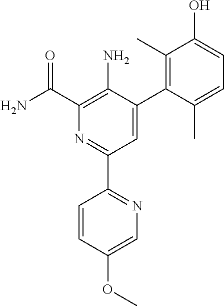 [SMILES: COc1ccc(-c2cc(-c3c(C)ccc(O)c3C)c(N)c(C(N)=O)n2)nc1] | 59 |
| 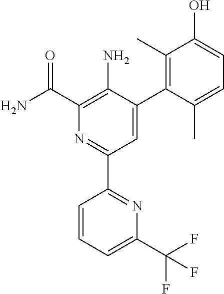 [SMILES: Cc1ccc(O)c(C)c1-c1cc(-c2cccc(C(F)(F)F)n2)nc(C(N)=O)c1N] | 60 |
| 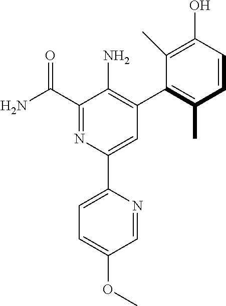 | 61 |
| 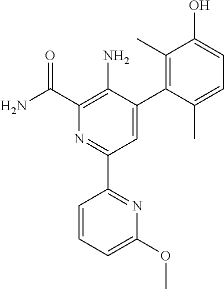 [SMILES: COc1cccc(-c2cc(-c3c(C)ccc(O)c3C)c(N)c(C(N)=O)n2)n1] | 62 |
| 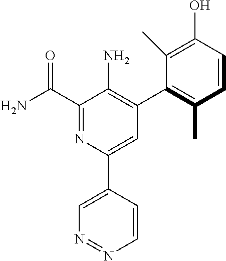 | 63 |
| 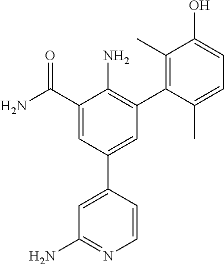 [SMILES: Cc1ccc(O)c(C)c1-c1cc(-c2ccnc(N)c2)cc(C(N)=O)c1N] | 64 |
| 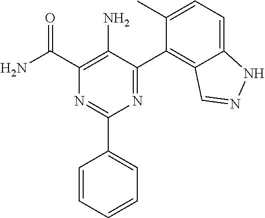 [SMILES: Cc1ccc2[nH]ncc2c1-c1nc(-c2ccccc2)nc(C(N)=O)c1N] | 65 |
| 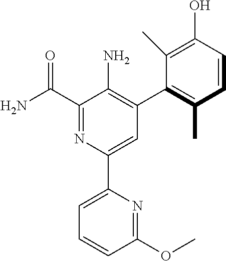 | 66 |
| 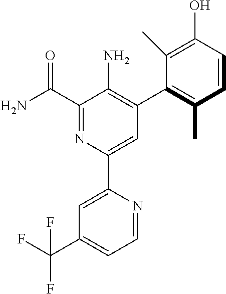 | 67 |
| 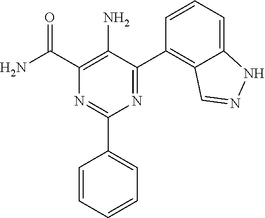 [SMILES: NC(=O)c1nc(-c2ccccc2)nc(-c2cccc3[nH]ncc23)c1N] | 68 |
| 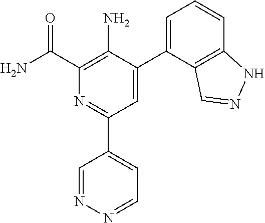 [SMILES: NC(=O)c1nc(-c2ccnnc2)cc(-c2cccc3[nH]ncc23)c1N] | 69 |
| 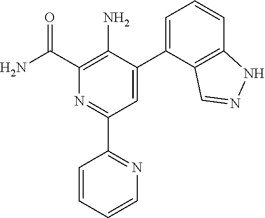 [SMILES: NC(=O)c1nc(-c2ccccn2)cc(-c2cccc3[nH]ncc23)c1N] | 70 |
| 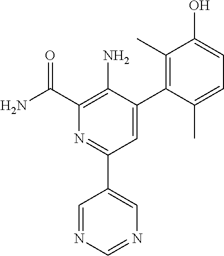 [SMILES: Cc1ccc(O)c(C)c1-c1cc(-c2cncnc2)nc(C(N)=O)c1N] | 71 |
| 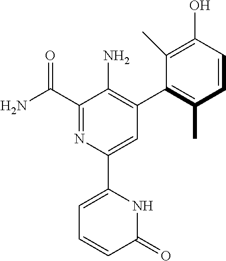 | 72 |
| 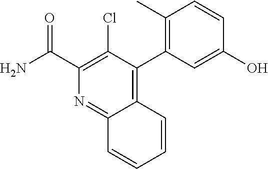 [SMILES: Cc1ccc(O)cc1-c1c(Cl)c(C(N)=O)nc2ccccc12] | 73 |
| 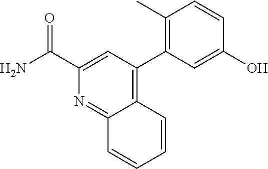 [SMILES: Cc1ccc(O)cc1-c1cc(C(N)=O)nc2ccccc12] | 74 |
| 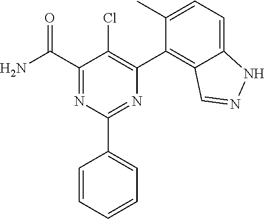 [SMILES: Cc1ccc2[nH]ncc2c1-c1nc(-c2ccccc2)nc(C(N)=O)c1Cl] | 75 |
| 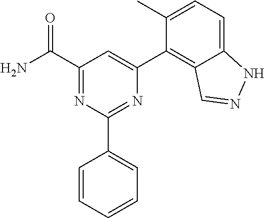 [SMILES: Cc1ccc2[nH]ncc2c1-c1cc(C(N)=O)nc(-c2ccccc2)n1] | 76 |
| 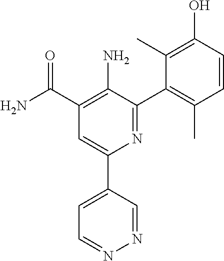 [SMILES: Cc1ccc(O)c(C)c1-c1nc(-c2ccnnc2)cc(C(N)=O)c1N] | 77 |
| 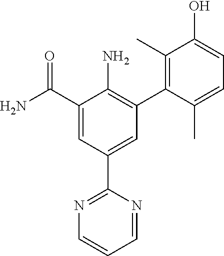 [SMILES: Cc1ccc(O)c(C)c1-c1cc(-c2ncccn2)cc(C(N)=O)c1N] | 78 |
| 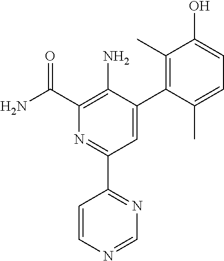 [SMILES: Cc1ccc(O)c(C)c1-c1cc(-c2ccncn2)nc(C(N)=O)c1N] | 79 |
| 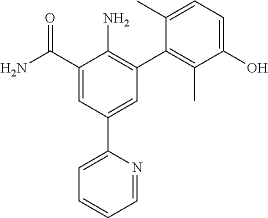 [SMILES: Cc1ccc(O)c(C)c1-c1cc(-c2ccccn2)cc(C(N)=O)c1N] | 80 |
| 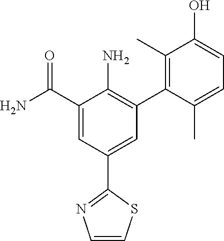 [SMILES: Cc1ccc(O)c(C)c1-c1cc(-c2nccs2)cc(C(N)=O)c1N] | 81 |
| 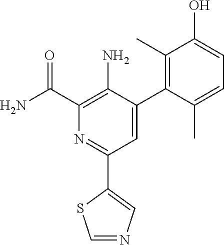 [SMILES: Cc1ccc(O)c(C)c1-c1cc(-c2cncs2)nc(C(N)=O)c1N] | 82 |
| 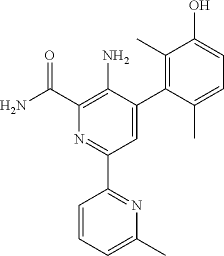 [SMILES: Cc1cccc(-c2cc(-c3c(C)ccc(O)c3C)c(N)c(C(N)=O)n2)n1] | 83 |
| 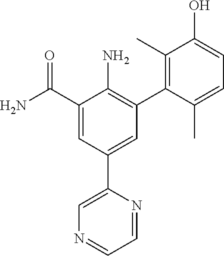 [SMILES: Cc1ccc(O)c(C)c1-c1cc(-c2cnccn2)cc(C(N)=O)c1N] | 84 |
| 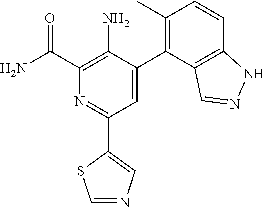 [SMILES: Cc1ccc2[nH]ncc2c1-c1cc(-c2cncs2)nc(C(N)=O)c1N] | 85 |
| 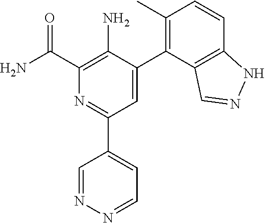 [SMILES: Cc1ccc2[nH]ncc2c1-c1cc(-c2ccnnc2)nc(C(N)=O)c1N] | 86 |
| 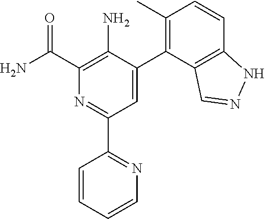 [SMILES: Cc1ccc2[nH]ncc2c1-c1cc(-c2ccccn2)nc(C(N)=O)c1N] | 87 |
| 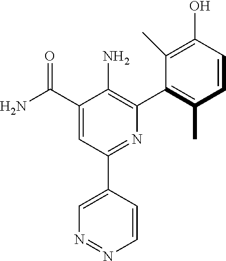 | 88 |
| 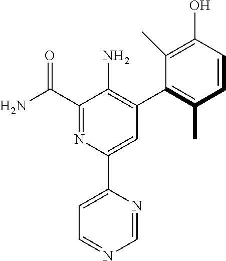 | 89 |
| 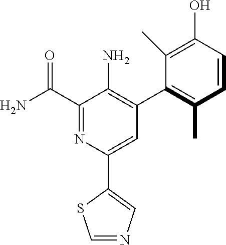 | 90 |
| 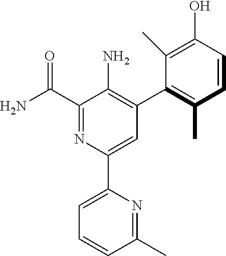 | 91 |
| 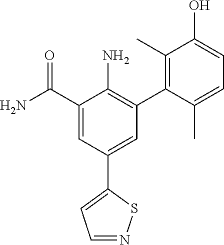 [SMILES: Cc1ccc(O)c(C)c1-c1cc(-c2ccns2)cc(C(N)=O)c1N] | 92 |
| 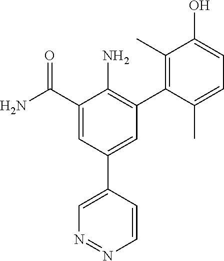 [SMILES: Cc1ccc(O)c(C)c1-c1cc(-c2ccnnc2)cc(C(N)=O)c1N] | 93 |
| 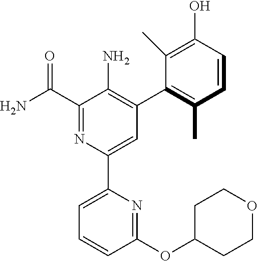 | 94 |
| 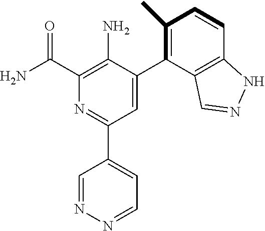 | 95 |
| 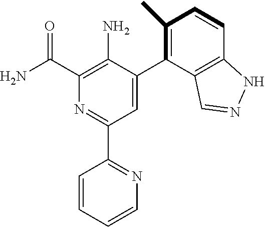 | 96 |
| 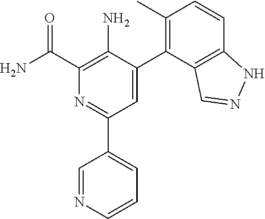 [SMILES: Cc1ccc2[nH]ncc2c1-c1cc(-c2cccnc2)nc(C(N)=O)c1N] | 97 |
| 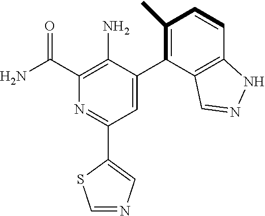 | 98 |
| 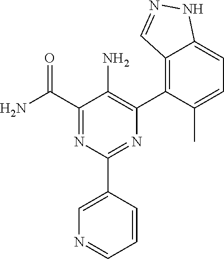 [SMILES: Cc1ccc2[nH]ncc2c1-c1nc(-c2cccnc2)nc(C(N)=O)c1N] | 99 |
| 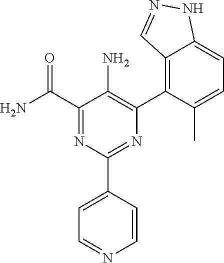 [SMILES: Cc1ccc2[nH]ncc2c1-c1nc(-c2ccncc2)nc(C(N)=O)c1N] | 100 |
| 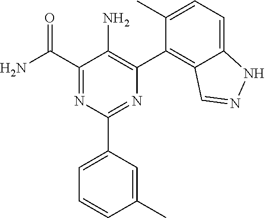 [SMILES: Cc1cccc(-c2nc(C(N)=O)c(N)c(-c3c(C)ccc4[nH]ncc34)n2)c1] | 101 |
| 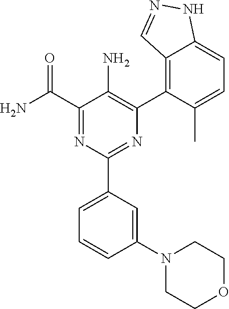 [SMILES: Cc1ccc2[nH]ncc2c1-c1nc(-c2cccc(N3CCOCC3)c2)nc(C(N)=O)c1N] | 102 |
| 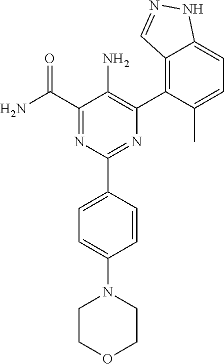 [SMILES: Cc1ccc2[nH]ncc2c1-c1nc(-c2ccc(N3CCOCC3)cc2)nc(C(N)=O)c1N] | 103 |
| 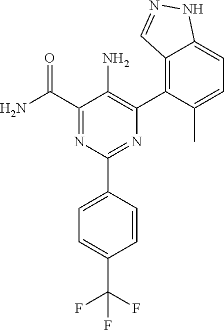 [SMILES: Cc1ccc2[nH]ncc2c1-c1nc(-c2ccc(C(F)(F)F)cc2)nc(C(N)=O)c1N] | 104 |
| 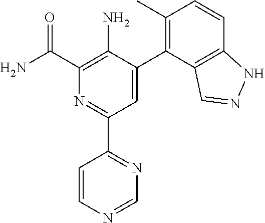 [SMILES: Cc1ccc2[nH]ncc2c1-c1cc(-c2ccncn2)nc(C(N)=O)c1N] | 105 |
| 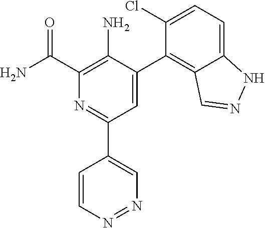 [SMILES: NC(=O)c1nc(-c2ccnnc2)cc(-c2c(Cl)ccc3[nH]ncc23)c1N] | 106 |
| 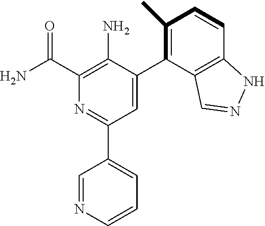 | 107 |
| 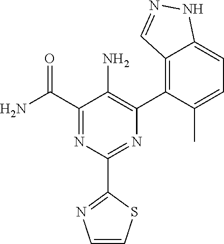 [SMILES: Cc1ccc2[nH]ncc2c1-c1nc(-c2nccs2)nc(C(N)=O)c1N] | 108 |
| 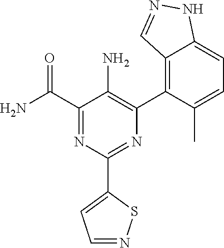 [SMILES: Cc1ccc2[nH]ncc2c1-c1nc(-c2ccns2)nc(C(N)=O)c1N] | 109 |
| 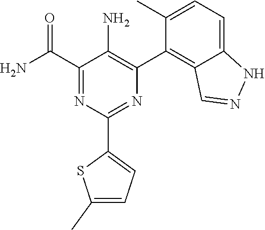 [SMILES: Cc1ccc(-c2nc(C(N)=O)c(N)c(-c3c(C)ccc4[nH]ncc34)n2)s1] | 110 |
| 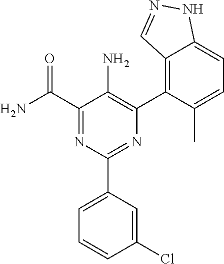 [SMILES: Cc1ccc2[nH]ncc2c1-c1nc(-c2cccc(Cl)c2)nc(C(N)=O)c1N] | 111 |
| 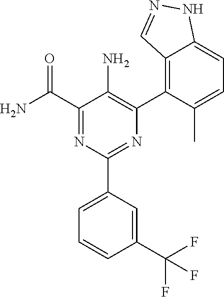 [SMILES: Cc1ccc2[nH]ncc2c1-c1nc(-c2cccc(C(F)(F)F)c2)nc(C(N)=O)c1N] | 112 |
| 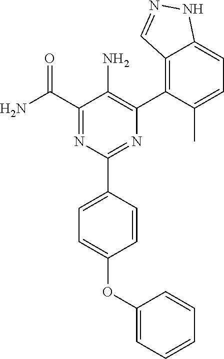 [SMILES: Cc1ccc2[nH]ncc2c1-c1nc(-c2ccc(Oc3ccccc3)cc2)nc(C(N)=O)c1N] | 113 |
| 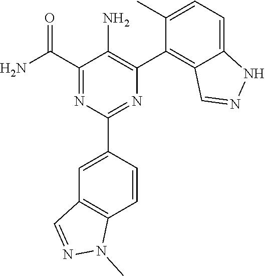 [SMILES: Cc1ccc2[nH]ncc2c1-c1nc(-c2ccc3c(cnn3C)c2)nc(C(N)=O)c1N] | 114 |
| 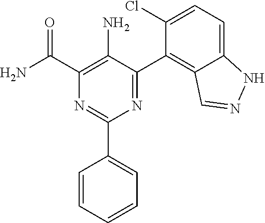 [SMILES: NC(=O)c1nc(-c2ccccc2)nc(-c2c(Cl)ccc3[nH]ncc23)c1N] | 115 |
| 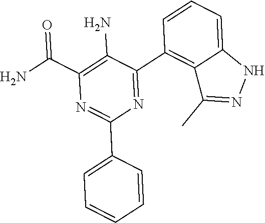 [SMILES: Cc1n[nH]c2cccc(-c3nc(-c4ccccc4)nc(C(N)=O)c3N)c12] | 116 |
| 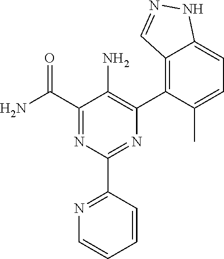 [SMILES: Cc1ccc2[nH]ncc2c1-c1nc(-c2ccccn2)nc(C(N)=O)c1N] | 117 |
| 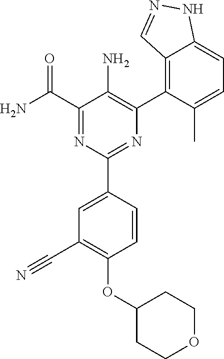 [SMILES: Cc1ccc2[nH]ncc2c1-c1nc(-c2ccc(OC3CCOCC3)c(C#N)c2)nc(C(N)=O)c1N] | 118 |
| 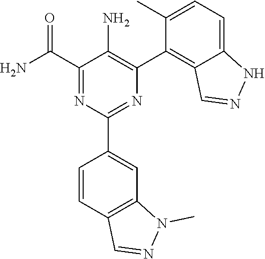 [SMILES: Cc1ccc2[nH]ncc2c1-c1nc(-c2ccc3cnn(C)c3c2)nc(C(N)=O)c1N] | 119 |
| 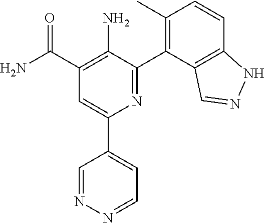 [SMILES: Cc1ccc2[nH]ncc2c1-c1nc(-c2ccnnc2)cc(C(N)=O)c1N] | 120 |
| 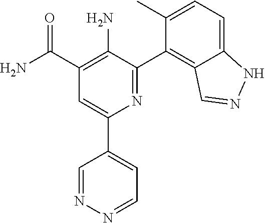 [SMILES: Cc1ccc2[nH]ncc2c1-c1nc(-c2ccnnc2)cc(C(N)=O)c1N] | 120 |
| 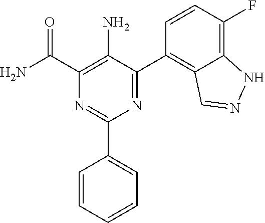 [SMILES: NC(=O)c1nc(-c2ccccc2)nc(-c2ccc(F)c3[nH]ncc23)c1N] | 121 |
| 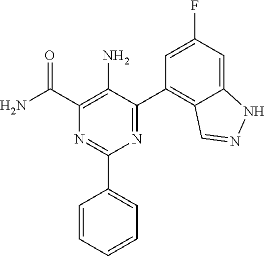 [SMILES: NC(=O)c1nc(-c2ccccc2)nc(-c2cc(F)cc3[nH]ncc23)c1N] | 122 |
| 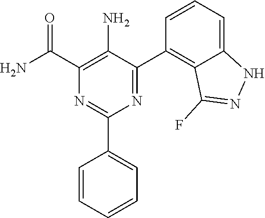 [SMILES: NC(=O)c1nc(-c2ccccc2)nc(-c2cccc3[nH]nc(F)c23)c1N] | 123 |
| 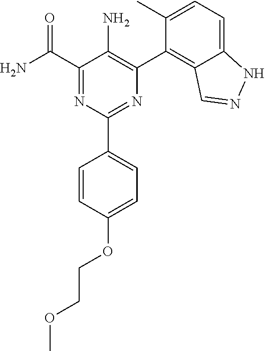 [SMILES: COCCOc1ccc(-c2nc(C(N)=O)c(N)c(-c3c(C)ccc4[nH]ncc34)n2)cc1] | 124 |
| 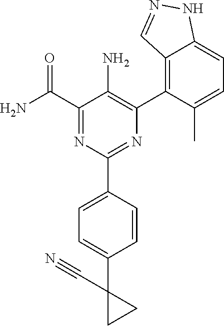 [SMILES: Cc1ccc2[nH]ncc2c1-c1nc(-c2ccc(C3(C#N)CC3)cc2)nc(C(N)=O)c1N] | 125 |
| 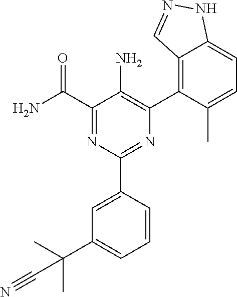 [SMILES: Cc1ccc2[nH]ncc2c1-c1nc(-c2cccc(C(C)(C)C#N)c2)nc(C(N)=O)c1N] | 126 |
| 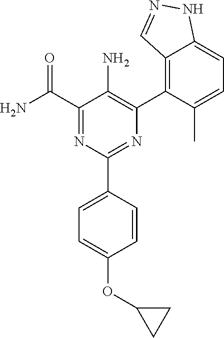 [SMILES: Cc1ccc2[nH]ncc2c1-c1nc(-c2ccc(OC3CC3)cc2)nc(C(N)=O)c1N] | 127 |
| 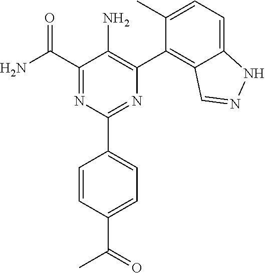 [SMILES: CC(=O)c1ccc(-c2nc(C(N)=O)c(N)c(-c3c(C)ccc4[nH]ncc34)n2)cc1] | 128 |
| 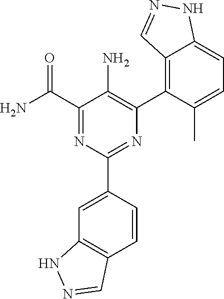 [SMILES: Cc1ccc2[nH]ncc2c1-c1nc(-c2ccc3cn[nH]c3c2)nc(C(N)=O)c1N] | 129 |
| 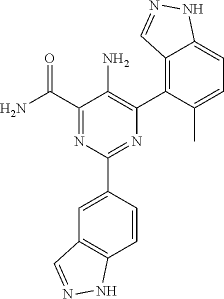 [SMILES: Cc1ccc2[nH]ncc2c1-c1nc(-c2ccc3[nH]ncc3c2)nc(C(N)=O)c1N] | 130 |
| 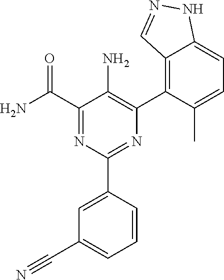 [SMILES: Cc1ccc2[nH]ncc2c1-c1nc(-c2cccc(C#N)c2)nc(C(N)=O)c1N] | 131 |
| 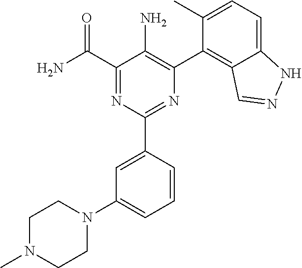 [SMILES: Cc1ccc2[nH]ncc2c1-c1nc(-c2cccc(N3CCN(C)CC3)c2)nc(C(N)=O)c1N] | 132 |
| 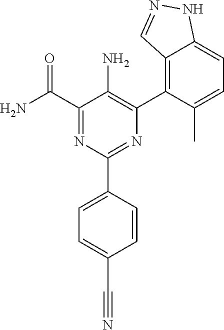 [SMILES: Cc1ccc2[nH]ncc2c1-c1nc(-c2ccc(C#N)cc2)nc(C(N)=O)c1N] | 133 |
| 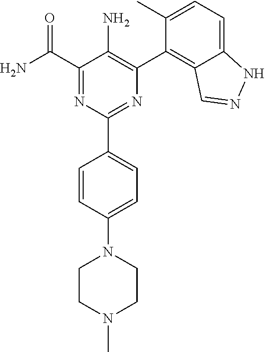 [SMILES: Cc1ccc2[nH]ncc2c1-c1nc(-c2ccc(N3CCN(C)CC3)cc2)nc(C(N)=O)c1N] | 134 |
| 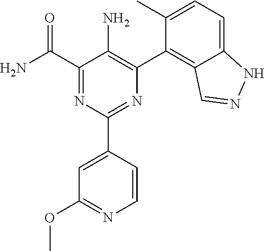 [SMILES: COc1cc(-c2nc(C(N)=O)c(N)c(-c3c(C)ccc4[nH]ncc34)n2)ccn1] | 135 |
| 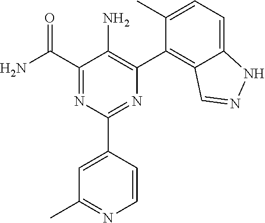 [SMILES: Cc1cc(-c2nc(C(N)=O)c(N)c(-c3c(C)ccc4[nH]ncc34)n2)ccn1] | 136 |
| 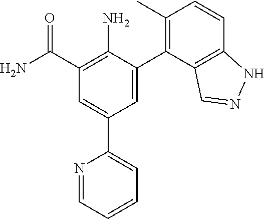 [SMILES: Cc1ccc2[nH]ncc2c1-c1cc(-c2ccccn2)cc(C(N)=O)c1N] | 137 |
| 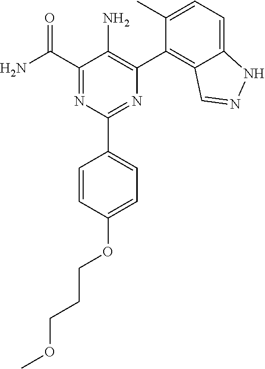 [SMILES: COCCCOc1ccc(-c2nc(C(N)=O)c(N)c(-c3c(C)ccc4[nH]ncc34)n2)cc1] | 138 |
| 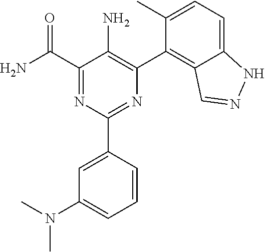 [SMILES: Cc1ccc2[nH]ncc2c1-c1nc(-c2cccc(N(C)C)c2)nc(C(N)=O)c1N] | 139 |
| 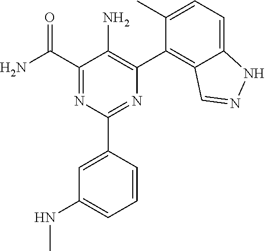 [SMILES: CNc1cccc(-c2nc(C(N)=O)c(N)c(-c3c(C)ccc4[nH]ncc34)n2)c1] | 140 |
| 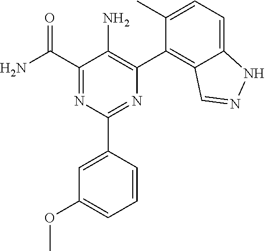 [SMILES: COc1cccc(-c2nc(C(N)=O)c(N)c(-c3c(C)ccc4[nH]ncc34)n2)c1] | 141 |
| 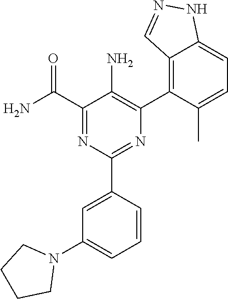 [SMILES: Cc1ccc2[nH]ncc2c1-c1nc(-c2cccc(N3CCCC3)c2)nc(C(N)=O)c1N] | 142 |
| 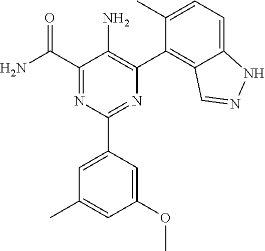 [SMILES: COc1cc(C)cc(-c2nc(C(N)=O)c(N)c(-c3c(C)ccc4[nH]ncc34)n2)c1] | 143 |
| 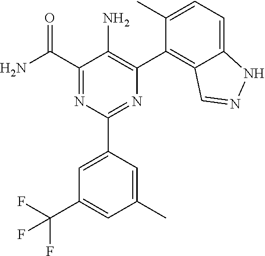 [SMILES: Cc1cc(-c2nc(C(N)=O)c(N)c(-c3c(C)ccc4[nH]ncc34)n2)cc(C(F)(F)F)c1] | 144 |
| 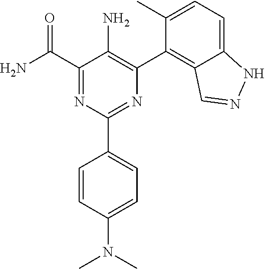 [SMILES: Cc1ccc2[nH]ncc2c1-c1nc(-c2ccc(N(C)C)cc2)nc(C(N)=O)c1N] | 145 |
| 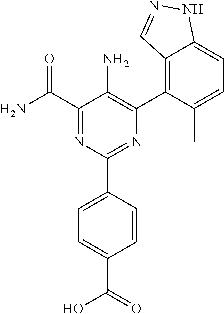 [SMILES: Cc1ccc2[nH]ncc2c1-c1nc(-c2ccc(C(=O)O)cc2)nc(C(N)=O)c1N] | 146 |
| 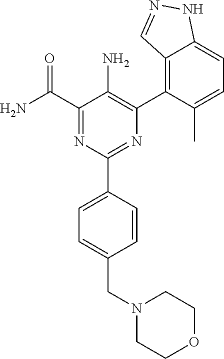 [SMILES: Cc1ccc2[nH]ncc2c1-c1nc(-c2ccc(CN3CCOCC3)cc2)nc(C(N)=O)c1N] | 147 |
| 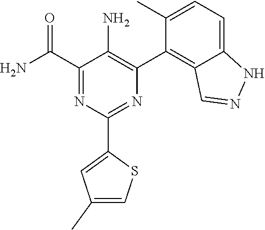 [SMILES: Cc1csc(-c2nc(C(N)=O)c(N)c(-c3c(C)ccc4[nH]ncc34)n2)c1] | 148 |
| 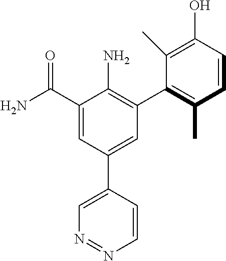 | 149 |
| 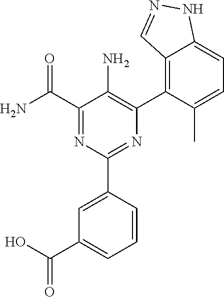 [SMILES: Cc1ccc2[nH]ncc2c1-c1nc(-c2cccc(C(=O)O)c2)nc(C(N)=O)c1N] | 150 |
| 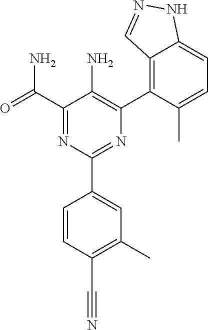 [SMILES: Cc1cc(-c2nc(C(N)=O)c(N)c(-c3c(C)ccc4[nH]ncc34)n2)ccc1C#N] | 151 |
| 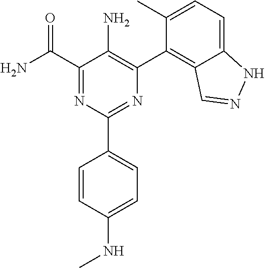 [SMILES: CNc1ccc(-c2nc(C(N)=O)c(N)c(-c3c(C)ccc4[nH]ncc34)n2)cc1] | 152 |
| 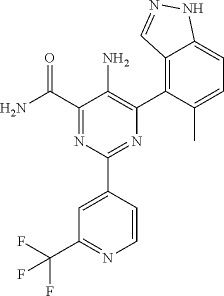 [SMILES: Cc1ccc2[nH]ncc2c1-c1nc(-c2ccnc(C(F)(F)F)c2)nc(C(N)=O)c1N] | 153 |
| 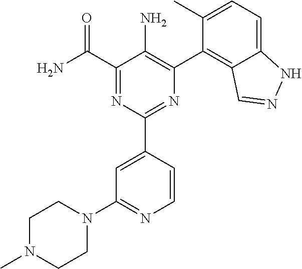 [SMILES: Cc1ccc2[nH]ncc2c1-c1nc(-c2ccnc(N3CCN(C)CC3)c2)nc(C(N)=O)c1N] | 154 |
| 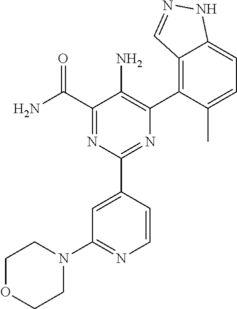 [SMILES: Cc1ccc2[nH]ncc2c1-c1nc(-c2ccnc(N3CCOCC3)c2)nc(C(N)=O)c1N] | 155 |
| 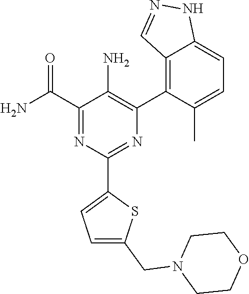 [SMILES: Cc1ccc2[nH]ncc2c1-c1nc(-c2ccc(CN3CCOCC3)s2)nc(C(N)=O)c1N] | 156 |
| 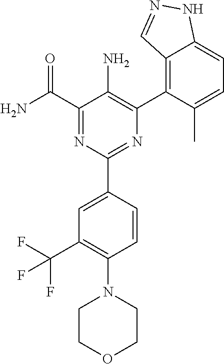 [SMILES: Cc1ccc2[nH]ncc2c1-c1nc(-c2ccc(N3CCOCC3)c(C(F)(F)F)c2)nc(C(N)=O)c1N] | 157 |
| 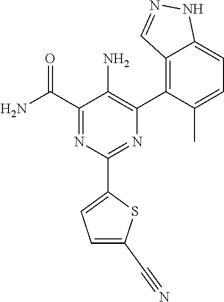 [SMILES: Cc1ccc2[nH]ncc2c1-c1nc(-c2ccc(C#N)s2)nc(C(N)=O)c1N] | 158 |
| 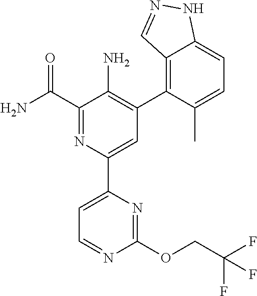 [SMILES: Cc1ccc2[nH]ncc2c1-c1cc(-c2ccnc(OCC(F)(F)F)n2)nc(C(N)=O)c1N] | 159 |
| 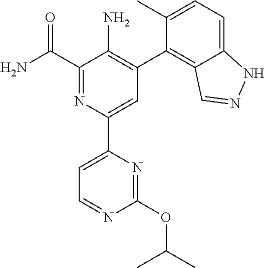 [SMILES: Cc1ccc2[nH]ncc2c1-c1cc(-c2ccnc(OC(C)C)n2)nc(C(N)=O)c1N] | 160 |
| 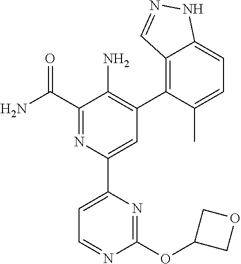 [SMILES: Cc1ccc2[nH]ncc2c1-c1cc(-c2ccnc(OC3COC3)n2)nc(C(N)=O)c1N] | 161 |
| 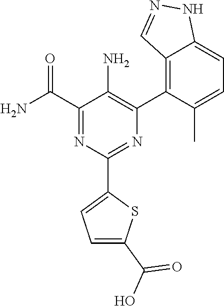 [SMILES: Cc1ccc2[nH]ncc2c1-c1nc(-c2ccc(C(=O)O)s2)nc(C(N)=O)c1N] | 162 |
| 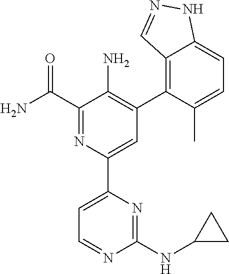 [SMILES: Cc1ccc2[nH]ncc2c1-c1cc(-c2ccnc(NC3CC3)n2)nc(C(N)=O)c1N] | 163 |
| 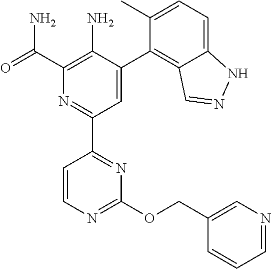 [SMILES: Cc1ccc2[nH]ncc2c1-c1cc(-c2ccnc(OCc3cccnc3)n2)nc(C(N)=O)c1N] | 164 |
| 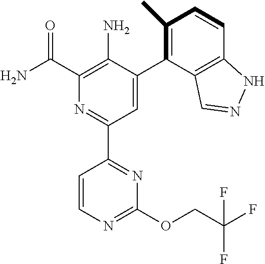 | 165 |
| 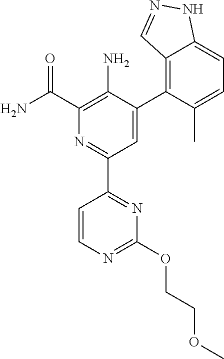 [SMILES: COCCOc1nccc(-c2cc(-c3c(C)ccc4[nH]ncc34)c(N)c(C(N)=O)n2)n1] | 166 |
| 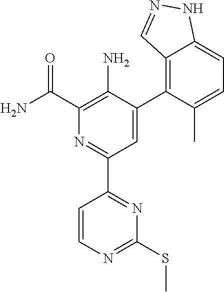 [SMILES: CSc1nccc(-c2cc(-c3c(C)ccc4[nH]ncc34)c(N)c(C(N)=O)n2)n1] | 167 |
| 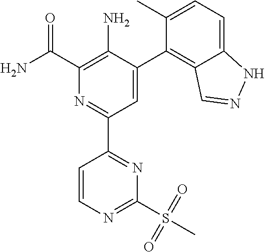 [SMILES: Cc1ccc2[nH]ncc2c1-c1cc(-c2ccnc(S(C)(=O)=O)n2)nc(C(N)=O)c1N] | 168 |
| 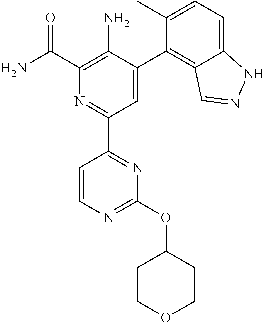 [SMILES: Cc1ccc2[nH]ncc2c1-c1cc(-c2ccnc(OC3CCOCC3)n2)nc(C(N)=O)c1N] | 169 |
| 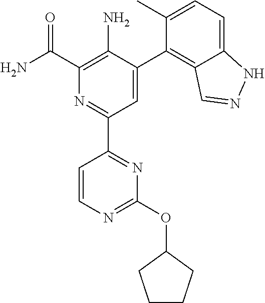 [SMILES: Cc1ccc2[nH]ncc2c1-c1cc(-c2ccnc(OC3CCCC3)n2)nc(C(N)=O)c1N] | 170 |
| 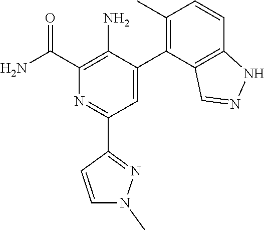 [SMILES: Cc1ccc2[nH]ncc2c1-c1cc(-c2ccn(C)n2)nc(C(N)=O)c1N] | 171 |
| 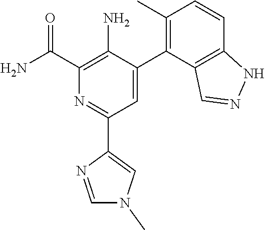 [SMILES: Cc1ccc2[nH]ncc2c1-c1cc(-c2cn(C)cn2)nc(C(N)=O)c1N] | 172 |
| 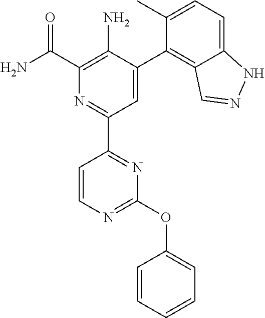 [SMILES: Cc1ccc2[nH]ncc2c1-c1cc(-c2ccnc(Oc3ccccc3)n2)nc(C(N)=O)c1N] | 173 |
| 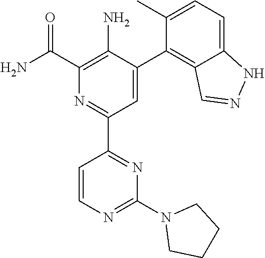 [SMILES: Cc1ccc2[nH]ncc2c1-c1cc(-c2ccnc(N3CCCC3)n2)nc(C(N)=O)c1N] | 174 |
| 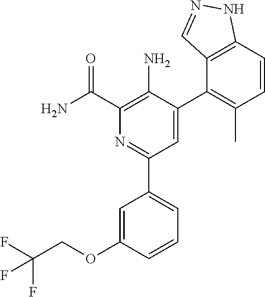 [SMILES: Cc1ccc2[nH]ncc2c1-c1cc(-c2cccc(OCC(F)(F)F)c2)nc(C(N)=O)c1N] | 175 |
| 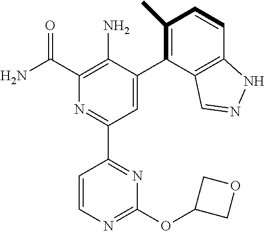 | 176 |
| 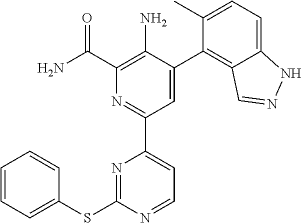 [SMILES: Cc1ccc2[nH]ncc2c1-c1cc(-c2ccnc(Sc3ccccc3)n2)nc(C(N)=O)c1N] | 177 |
| 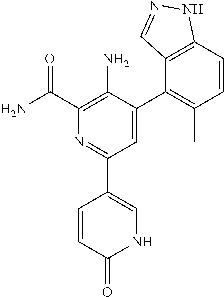 [SMILES: Cc1ccc2[nH]ncc2c1-c1cc(-c2ccc(=O)[nH]c2)nc(C(N)=O)c1N] | 178 |
| 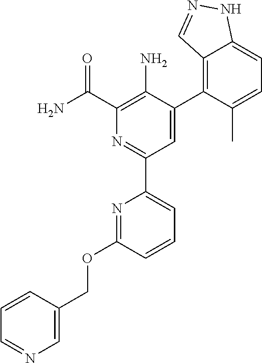 [SMILES: Cc1ccc2[nH]ncc2c1-c1cc(-c2cccc(OCc3cccnc3)n2)nc(C(N)=O)c1N] | 179 |
| 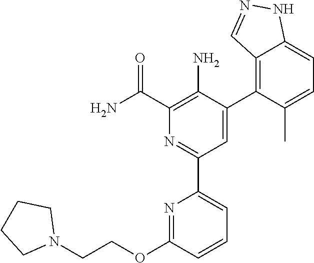 [SMILES: Cc1ccc2[nH]ncc2c1-c1cc(-c2cccc(OCCN3CCCC3)n2)nc(C(N)=O)c1N] | 180 |
| 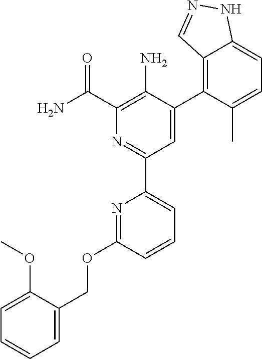 [SMILES: COc1ccccc1COc1cccc(-c2cc(-c3c(C)ccc4[nH]ncc34)c(N)c(C(N)=O)n2)n1] | 181 |
| 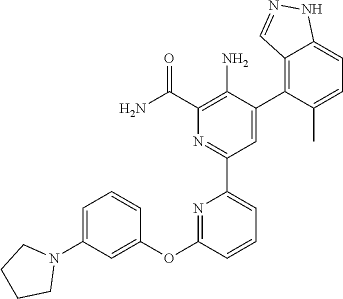 [SMILES: Cc1ccc2[nH]ncc2c1-c1cc(-c2cccc(Oc3cccc(N4CCCC4)c3)n2)nc(C(N)=O)c1N] | 182 |
| 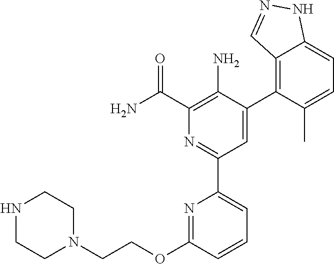 [SMILES: Cc1ccc2[nH]ncc2c1-c1cc(-c2cccc(OCCN3CCNCC3)n2)nc(C(N)=O)c1N] | 183 |
| 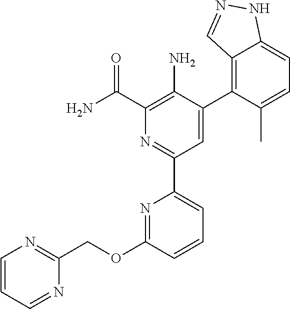 [SMILES: Cc1ccc2[nH]ncc2c1-c1cc(-c2cccc(OCc3ncccn3)n2)nc(C(N)=O)c1N] | 184 |
| 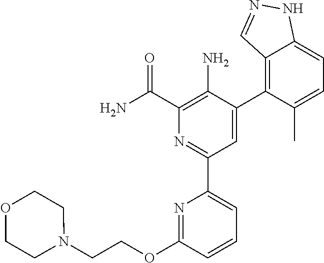 [SMILES: Cc1ccc2[nH]ncc2c1-c1cc(-c2cccc(OCCN3CCOCC3)n2)nc(C(N)=O)c1N] | 185 |
| 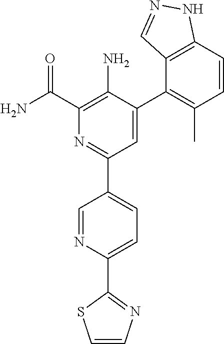 [SMILES: Cc1ccc2[nH]ncc2c1-c1cc(-c2ccc(-c3nccs3)nc2)nc(C(N)=O)c1N] | 186 |
| 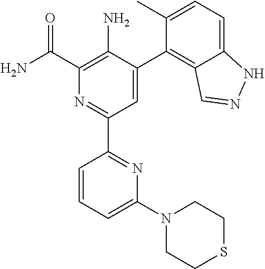 [SMILES: Cc1ccc2[nH]ncc2c1-c1cc(-c2cccc(N3CCSCC3)n2)nc(C(N)=O)c1N] | 187 |
| 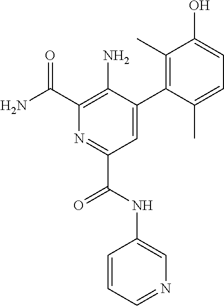 [SMILES: Cc1ccc(O)c(C)c1-c1cc(C(=O)Nc2cccnc2)nc(C(N)=O)c1N] | 188 |
| 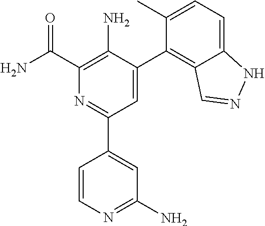 [SMILES: Cc1ccc2[nH]ncc2c1-c1cc(-c2ccnc(N)c2)nc(C(N)=O)c1N] | 189 |
| 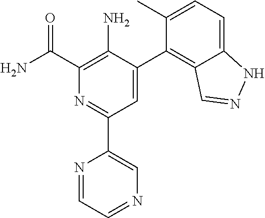 [SMILES: Cc1ccc2[nH]ncc2c1-c1cc(-c2cnccn2)nc(C(N)=O)c1N] | 190 |
| 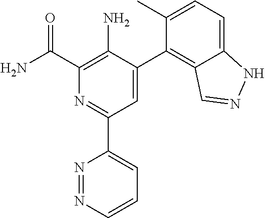 [SMILES: Cc1ccc2[nH]ncc2c1-c1cc(-c2cccnn2)nc(C(N)=O)c1N] | 191 |
| 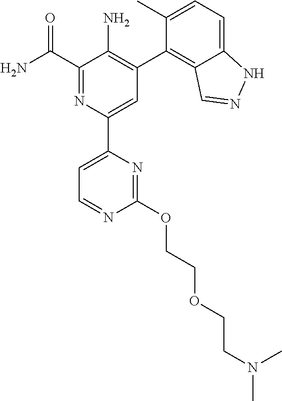 [SMILES: Cc1ccc2[nH]ncc2c1-c1cc(-c2ccnc(OCCOCCN(C)C)n2)nc(C(N)=O)c1N] | 192 |
| 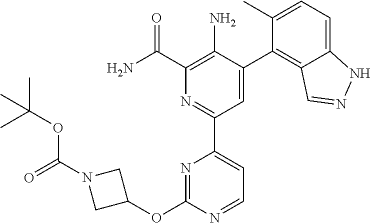 [SMILES: Cc1ccc2[nH]ncc2c1-c1cc(-c2ccnc(OC3CN(C(=O)OC(C)(C)C)C3)n2)nc(C(N)=O)c1N] | 193 |
| 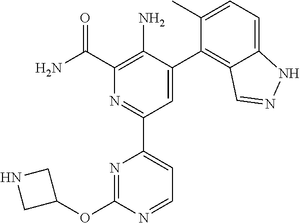 [SMILES: Cc1ccc2[nH]ncc2c1-c1cc(-c2ccnc(OC3CNC3)n2)nc(C(N)=O)c1N] | 194 |
| 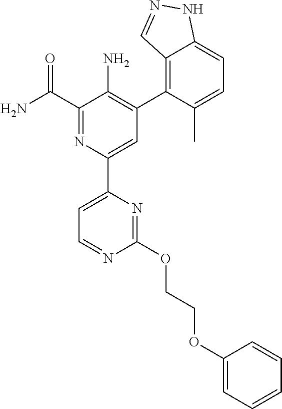 [SMILES: Cc1ccc2[nH]ncc2c1-c1cc(-c2ccnc(OCCOc3ccccc3)n2)nc(C(N)=O)c1N] | 195 |
| 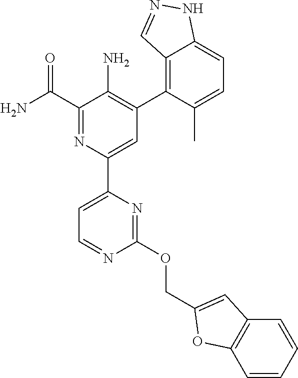 [SMILES: Cc1ccc2[nH]ncc2c1-c1cc(-c2ccnc(OCc3cc4ccccc4o3)n2)nc(C(N)=O)c1N] | 196 |
| 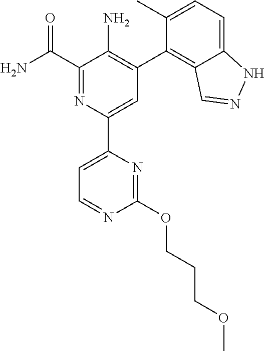 [SMILES: COCCCOc1nccc(-c2cc(-c3c(C)ccc4[nH]ncc34)c(N)c(C(N)=O)n2)n1] | 197 |
| 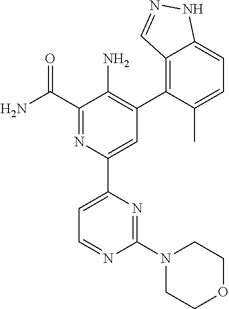 [SMILES: Cc1ccc2[nH]ncc2c1-c1cc(-c2ccnc(N3CCOCC3)n2)nc(C(N)=O)c1N] | 198 |
| 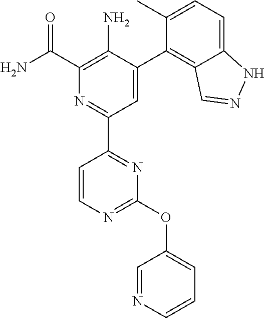 [SMILES: Cc1ccc2[nH]ncc2c1-c1cc(-c2ccnc(Oc3cccnc3)n2)nc(C(N)=O)c1N] | 199 |
| 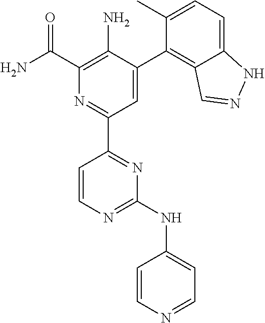 [SMILES: Cc1ccc2[nH]ncc2c1-c1cc(-c2ccnc(Nc3ccncc3)n2)nc(C(N)=O)c1N] | 200 |
| 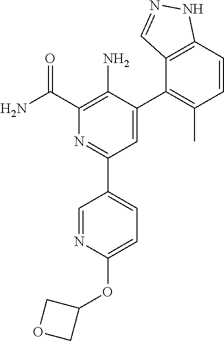 [SMILES: Cc1ccc2[nH]ncc2c1-c1cc(-c2ccc(OC3COC3)nc2)nc(C(N)=O)c1N] | 201 |
| 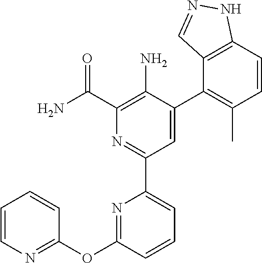 [SMILES: Cc1ccc2[nH]ncc2c1-c1cc(-c2cccc(Oc3ccccn3)n2)nc(C(N)=O)c1N] | 202 |
| 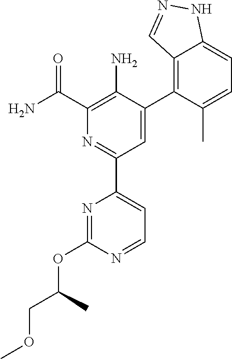 [SMILES: COC[C@H](C)Oc1nccc(-c2cc(-c3c(C)ccc4[nH]ncc34)c(N)c(C(N)=O)n2)n1] | 203 |
| 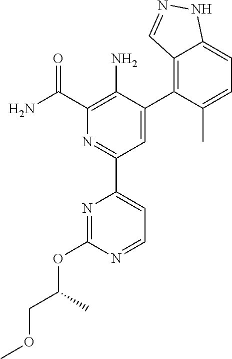 [SMILES: COC[C@@H](C)Oc1nccc(-c2cc(-c3c(C)ccc4[nH]ncc34)c(N)c(C(N)=O)n2)n1] | 204 |
| 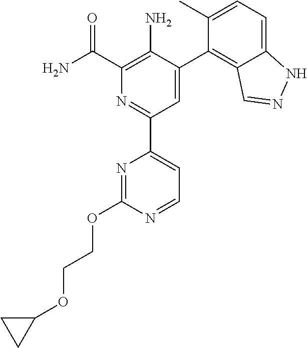 [SMILES: Cc1ccc2[nH]ncc2c1-c1cc(-c2ccnc(OCCOC3CC3)n2)nc(C(N)=O)c1N] | 205 |
| 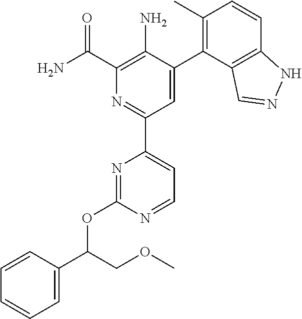 [SMILES: COCC(Oc1nccc(-c2cc(-c3c(C)ccc4[nH]ncc34)c(N)c(C(N)=O)n2)n1)c1ccccc1] | 206 |
| 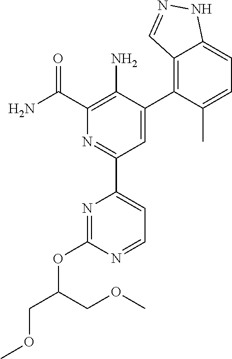 [SMILES: COCC(COC)Oc1nccc(-c2cc(-c3c(C)ccc4[nH]ncc34)c(N)c(C(N)=O)n2)n1] | 207 |
| 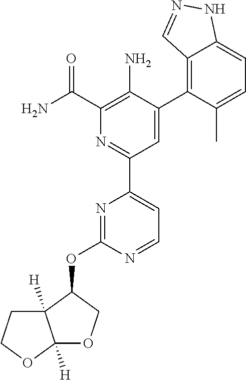 [SMILES: Cc1ccc2[nH]ncc2c1-c1cc(-c2ccnc(O[C@H]3CO[C@H]4OCC[C@H]43)n2)nc(C(N)=O)c1N] | 208 |
| 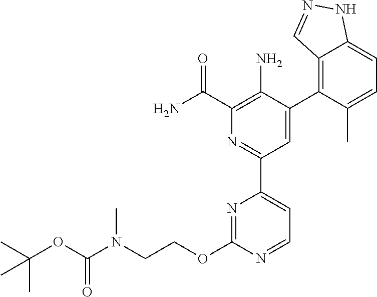 [SMILES: Cc1ccc2[nH]ncc2c1-c1cc(-c2ccnc(OCCN(C)C(=O)OC(C)(C)C)n2)nc(C(N)=O)c1N] | 209 |
| 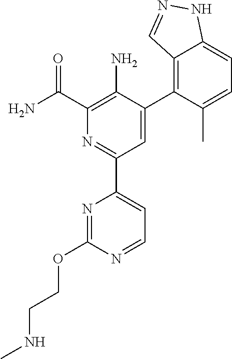 [SMILES: CNCCOc1nccc(-c2cc(-c3c(C)ccc4[nH]ncc34)c(N)c(C(N)=O)n2)n1] | 210 |
| 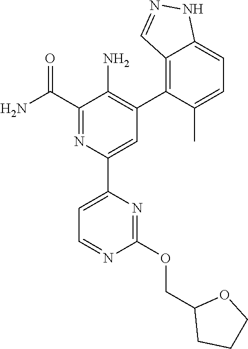 [SMILES: Cc1ccc2[nH]ncc2c1-c1cc(-c2ccnc(OCC3CCCO3)n2)nc(C(N)=O)c1N] | 211 |
| 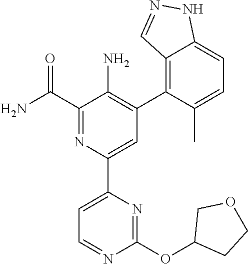 [SMILES: Cc1ccc2[nH]ncc2c1-c1cc(-c2ccnc(OC3CCOC3)n2)nc(C(N)=O)c1N] | 212 |
| 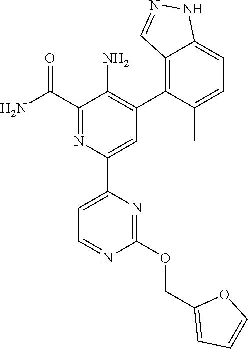 [SMILES: Cc1ccc2[nH]ncc2c1-c1cc(-c2ccnc(OCc3ccco3)n2)nc(C(N)=O)c1N] | 213 |
| 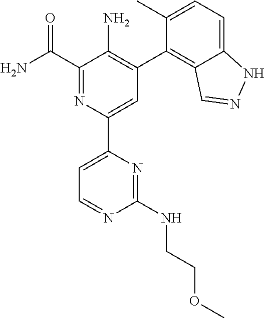 [SMILES: COCCNc1nccc(-c2cc(-c3c(C)ccc4[nH]ncc34)c(N)c(C(N)=O)n2)n1] | 214 |
| 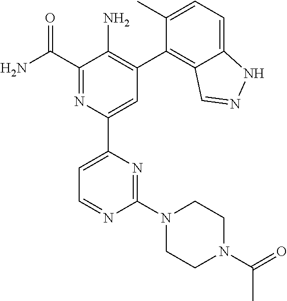 [SMILES: CC(=O)N1CCN(c2nccc(-c3cc(-c4c(C)ccc5[nH]ncc45)c(N)c(C(N)=O)n3)n2)CC1] | 215 |
| 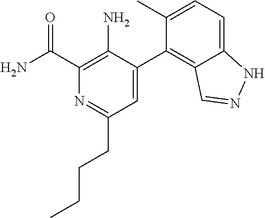 [SMILES: CCCCc1cc(-c2c(C)ccc3[nH]ncc23)c(N)c(C(N)=O)n1] | 216 |
| 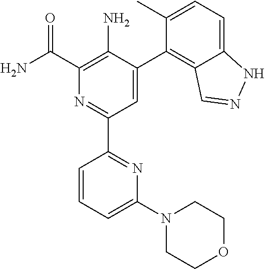 [SMILES: Cc1ccc2[nH]ncc2c1-c1cc(-c2cccc(N3CCOCC3)n2)nc(C(N)=O)c1N] | 217 |
| 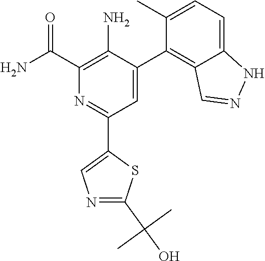 [SMILES: Cc1ccc2[nH]ncc2c1-c1cc(-c2cnc(C(C)(C)O)s2)nc(C(N)=O)c1N] | 218 |
| 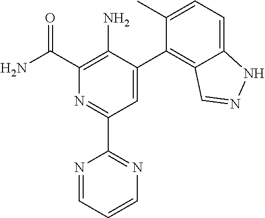 [SMILES: Cc1ccc2[nH]ncc2c1-c1cc(-c2ncccn2)nc(C(N)=O)c1N] | 219 |
| 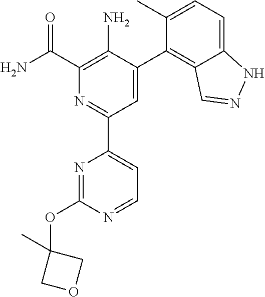 [SMILES: Cc1ccc2[nH]ncc2c1-c1cc(-c2ccnc(OC3(C)COC3)n2)nc(C(N)=O)c1N] | 220 |
| 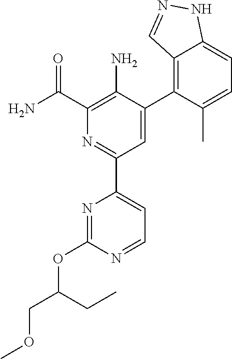 [SMILES: CCC(COC)Oc1nccc(-c2cc(-c3c(C)ccc4[nH]ncc34)c(N)c(C(N)=O)n2)n1] | 221 |
| 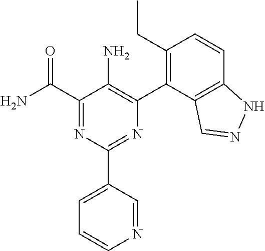 [SMILES: CCc1ccc2[nH]ncc2c1-c1nc(-c2cccnc2)nc(C(N)=O)c1N] | 222 |
| 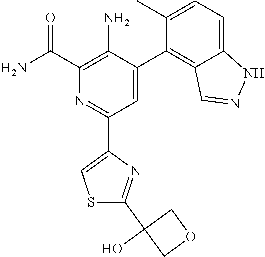 [SMILES: Cc1ccc2[nH]ncc2c1-c1cc(-c2csc(C3(O)COC3)n2)nc(C(N)=O)c1N] | 223 |
| 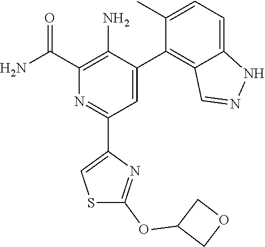 [SMILES: Cc1ccc2[nH]ncc2c1-c1cc(-c2csc(OC3COC3)n2)nc(C(N)=O)c1N] | 224 |
| 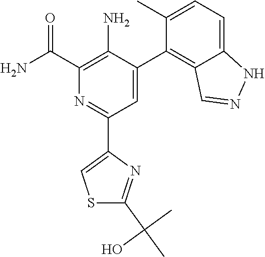 [SMILES: Cc1ccc2[nH]ncc2c1-c1cc(-c2csc(C(C)(C)O)n2)nc(C(N)=O)c1N] | 225 |
| 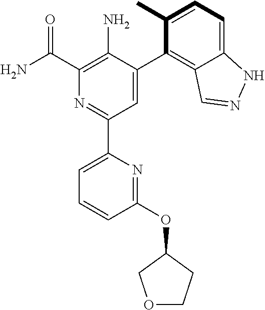 | 226 |
| 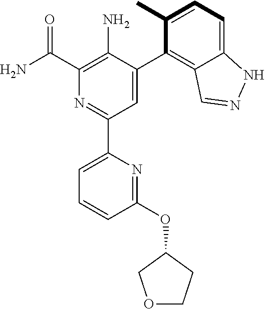 | 227 |
| 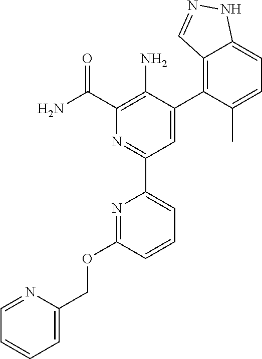 [SMILES: Cc1ccc2[nH]ncc2c1-c1cc(-c2cccc(OCc3ccccn3)n2)nc(C(N)=O)c1N] | 228 |
| 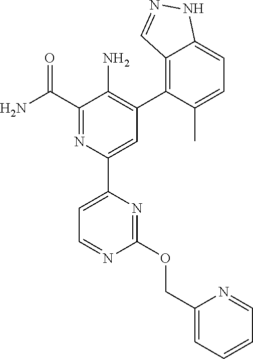 [SMILES: Cc1ccc2[nH]ncc2c1-c1cc(-c2ccnc(OCc3ccccn3)n2)nc(C(N)=O)c1N] | 229 |
| 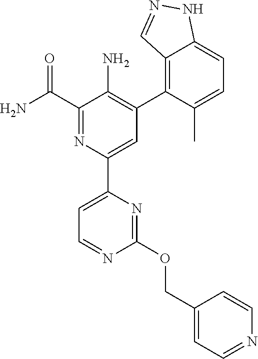 [SMILES: Cc1ccc2[nH]ncc2c1-c1cc(-c2ccnc(OCc3ccncc3)n2)nc(C(N)=O)c1N] | 230 |
| 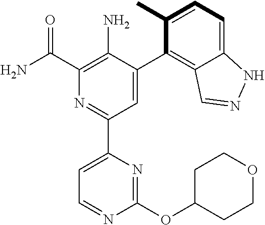 | 231 |
| 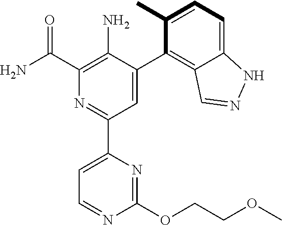 | 232 |
| 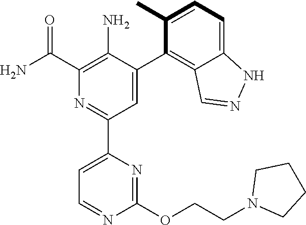 | 233 |
| 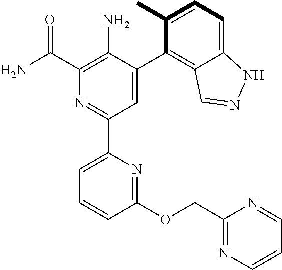 | 234 |
| 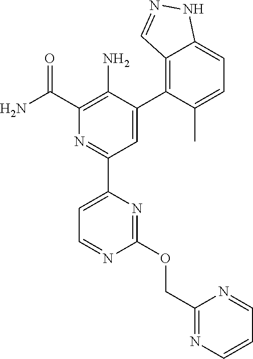 [SMILES: Cc1ccc2[nH]ncc2c1-c1cc(-c2ccnc(OCc3ncccn3)n2)nc(C(N)=O)c1N] | 235 |
| 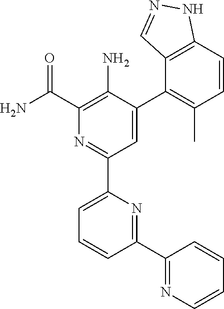 [SMILES: Cc1ccc2[nH]ncc2c1-c1cc(-c2cccc(-c3ccccn3)n2)nc(C(N)=O)c1N] | 236 |
| 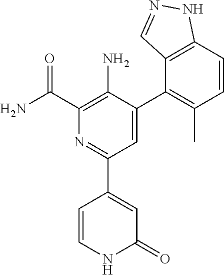 [SMILES: Cc1ccc2[nH]ncc2c1-c1cc(-c2cc[nH]c(=O)c2)nc(C(N)=O)c1N] | 237 |
| 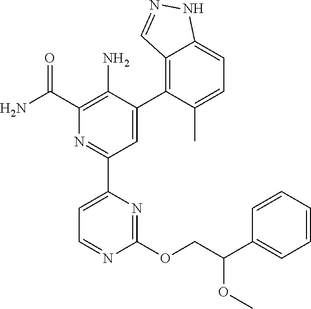 [SMILES: COC(COc1nccc(-c2cc(-c3c(C)ccc4[nH]ncc34)c(N)c(C(N)=O)n2)n1)c1ccccc1] | 238 |
| 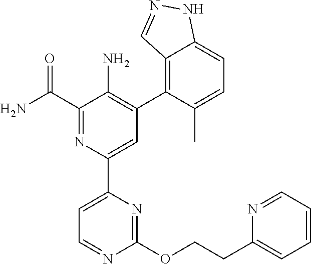 [SMILES: Cc1ccc2[nH]ncc2c1-c1cc(-c2ccnc(OCCc3ccccn3)n2)nc(C(N)=O)c1N] | 239 |
| 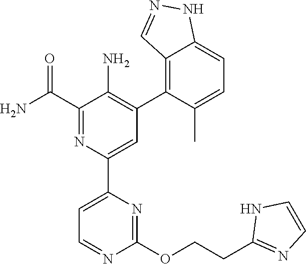 [SMILES: Cc1ccc2[nH]ncc2c1-c1cc(-c2ccnc(OCCc3ncc[nH]3)n2)nc(C(N)=O)c1N] | 240 |
| 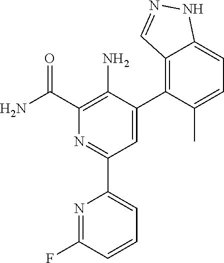 [SMILES: Cc1ccc2[nH]ncc2c1-c1cc(-c2cccc(F)n2)nc(C(N)=O)c1N] | 241 |
| 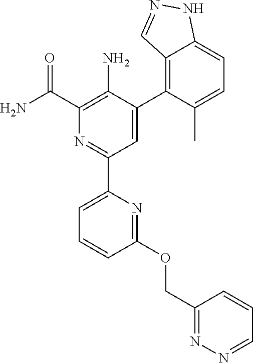 [SMILES: Cc1ccc2[nH]ncc2c1-c1cc(-c2cccc(OCc3cccnn3)n2)nc(C(N)=O)c1N] | 242 |
| 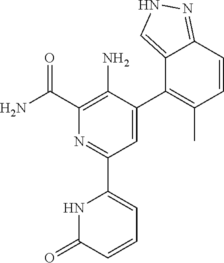 [SMILES: Cc1ccc2n[nH]cc2c1-c1cc(-c2cccc(=O)[nH]2)nc(C(N)=O)c1N] | 243 |
| 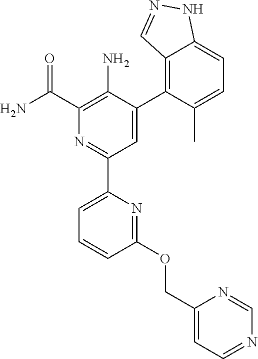 [SMILES: Cc1ccc2[nH]ncc2c1-c1cc(-c2cccc(OCc3ccncn3)n2)nc(C(N)=O)c1N] | 244 |
| 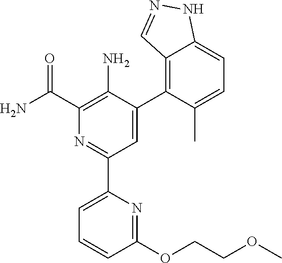 [SMILES: COCCOc1cccc(-c2cc(-c3c(C)ccc4[nH]ncc34)c(N)c(C(N)=O)n2)n1] | 245 |
| 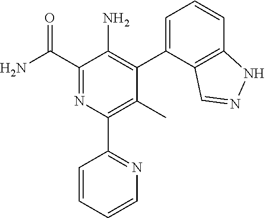 [SMILES: Cc1c(-c2ccccn2)nc(C(N)=O)c(N)c1-c1cccc2[nH]ncc12] | 246 |
| 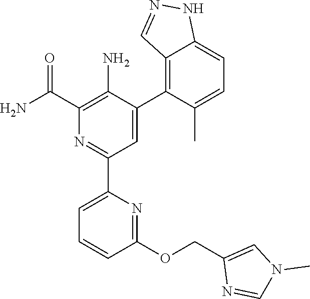 [SMILES: Cc1ccc2[nH]ncc2c1-c1cc(-c2cccc(OCc3cn(C)cn3)n2)nc(C(N)=O)c1N] | 247 |
| 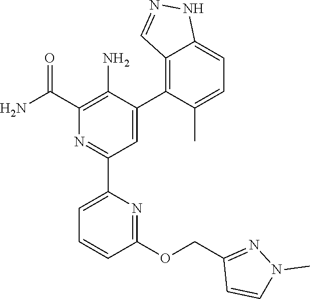 [SMILES: Cc1ccc2[nH]ncc2c1-c1cc(-c2cccc(OCc3ccn(C)n3)n2)nc(C(N)=O)c1N] | 248 |
| 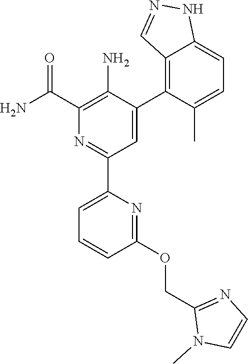 [SMILES: Cc1ccc2[nH]ncc2c1-c1cc(-c2cccc(OCc3nccn3C)n2)nc(C(N)=O)c1N] | 249 |
| 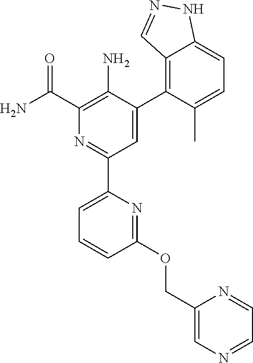 [SMILES: Cc1ccc2[nH]ncc2c1-c1cc(-c2cccc(OCc3cnccn3)n2)nc(C(N)=O)c1N] | 250 |
| 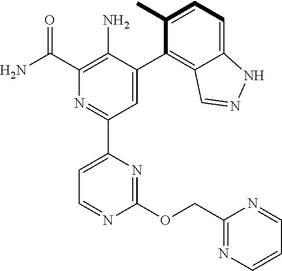 | 251 |
| 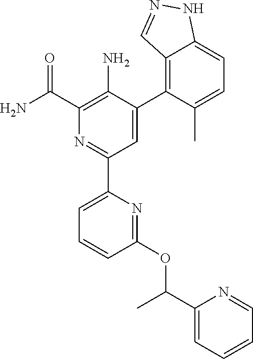 [SMILES: Cc1ccc2[nH]ncc2c1-c1cc(-c2cccc(OC(C)c3ccccn3)n2)nc(C(N)=O)c1N] | 252 |
| 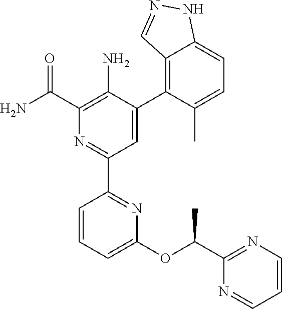 [SMILES: Cc1ccc2[nH]ncc2c1-c1cc(-c2cccc(O[C@@H](C)c3ncccn3)n2)nc(C(N)=O)c1N] | 253 |
| 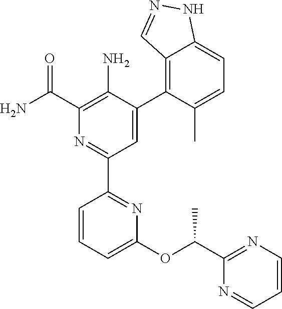 [SMILES: Cc1ccc2[nH]ncc2c1-c1cc(-c2cccc(O[C@H](C)c3ncccn3)n2)nc(C(N)=O)c1N] | 254 |
| 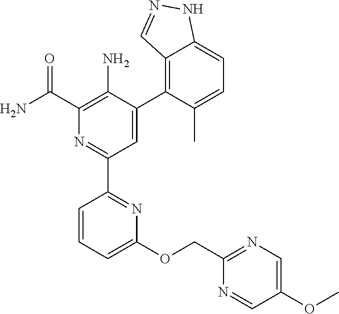 [SMILES: COc1cnc(COc2cccc(-c3cc(-c4c(C)ccc5[nH]ncc45)c(N)c(C(N)=O)n3)n2)nc1] | 255 |
| 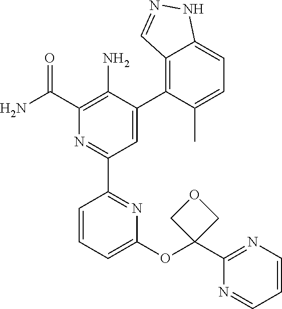 [SMILES: Cc1ccc2[nH]ncc2c1-c1cc(-c2cccc(OC3(c4ncccn4)COC3)n2)nc(C(N)=O)c1N] | 256 |
| 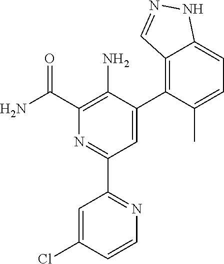 [SMILES: Cc1ccc2[nH]ncc2c1-c1cc(-c2cc(Cl)ccn2)nc(C(N)=O)c1N] | 257 |
| 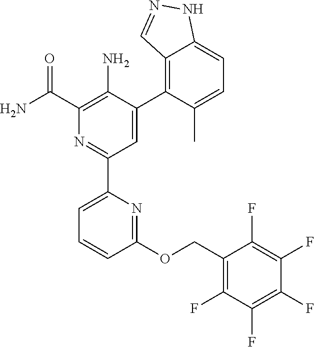 [SMILES: Cc1ccc2[nH]ncc2c1-c1cc(-c2cccc(OCc3c(F)c(F)c(F)c(F)c3F)n2)nc(C(N)=O)c1N] | 258 |
| 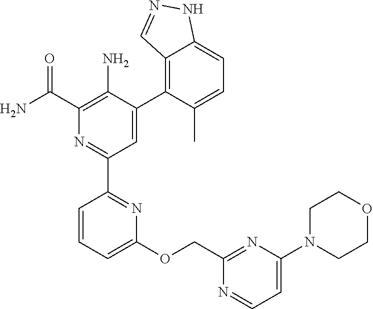 [SMILES: Cc1ccc2[nH]ncc2c1-c1cc(-c2cccc(OCc3nccc(N4CCOCC4)n3)n2)nc(C(N)=O)c1N] | 259 |
| 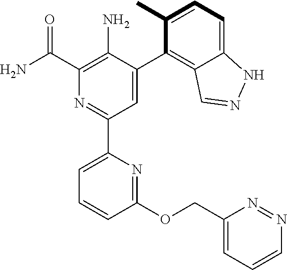 | 260 |
| 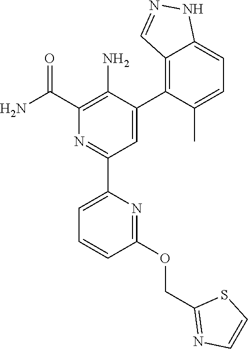 [SMILES: Cc1ccc2[nH]ncc2c1-c1cc(-c2cccc(OCc3nccs3)n2)nc(C(N)=O)c1N] | 261 |
| 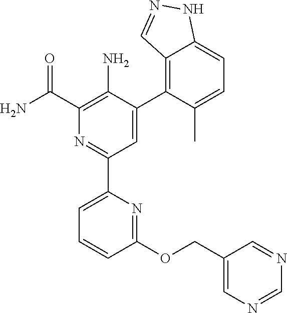 [SMILES: Cc1ccc2[nH]ncc2c1-c1cc(-c2cccc(OCc3cncnc3)n2)nc(C(N)=O)c1N] | 262 |
| 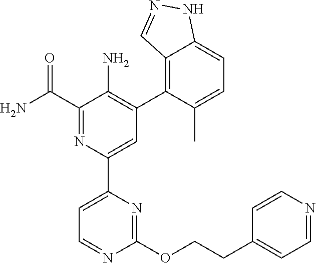 [SMILES: Cc1ccc2[nH]ncc2c1-c1cc(-c2ccnc(OCCc3ccncc3)n2)nc(C(N)=O)c1N] | 263 |
| 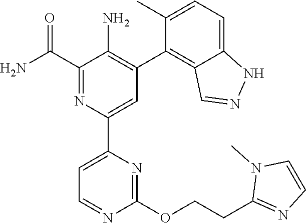 [SMILES: Cc1ccc2[nH]ncc2c1-c1cc(-c2ccnc(OCCc3nccn3C)n2)nc(C(N)=O)c1N] | 264 |
| 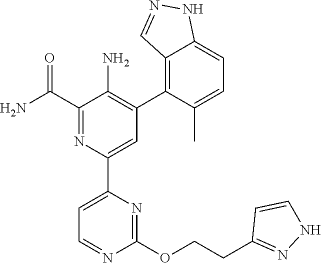 [SMILES: Cc1ccc2[nH]ncc2c1-c1cc(-c2ccnc(OCCc3cc[nH]n3)n2)nc(C(N)=O)c1N] | 265 |
| 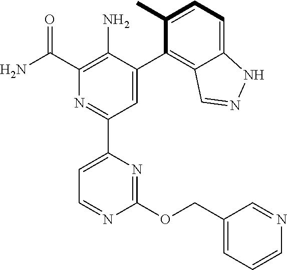 | 266 |
| 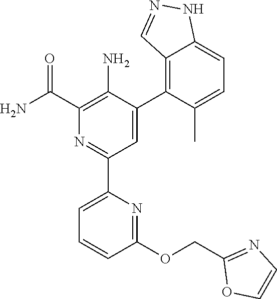 [SMILES: Cc1ccc2[nH]ncc2c1-c1cc(-c2cccc(OCc3ncco3)n2)nc(C(N)=O)c1N] | 267 |
| 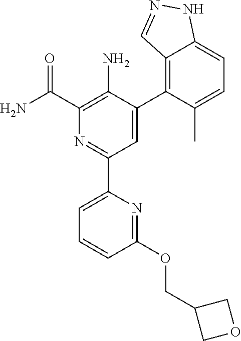 [SMILES: Cc1ccc2[nH]ncc2c1-c1cc(-c2cccc(OCC3COC3)n2)nc(C(N)=O)c1N] | 268 |
| 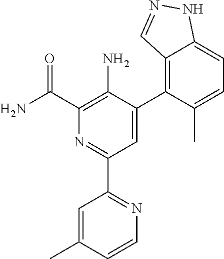 [SMILES: Cc1ccnc(-c2cc(-c3c(C)ccc4[nH]ncc34)c(N)c(C(N)=O)n2)c1] | 269 |
| 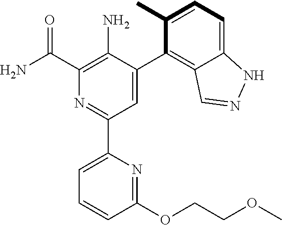 | 270 |
| 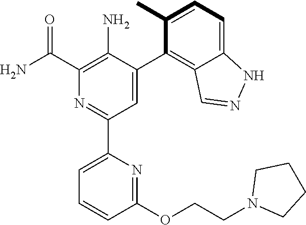 | 271 |
| 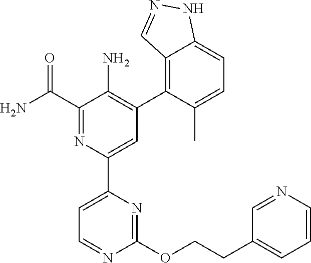 [SMILES: Cc1ccc2[nH]ncc2c1-c1cc(-c2ccnc(OCCc3cccnc3)n2)nc(C(N)=O)c1N] | 272 |
| 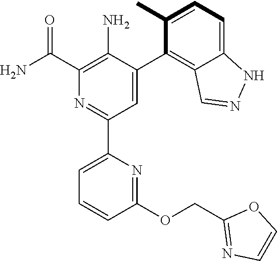 | 273 |
| 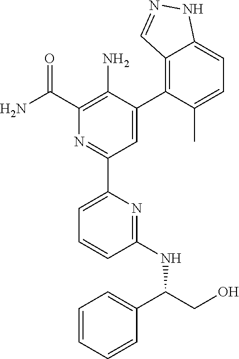 [SMILES: Cc1ccc2[nH]ncc2c1-c1cc(-c2cccc(N[C@H](CO)c3ccccc3)n2)nc(C(N)=O)c1N] | 274 |
| 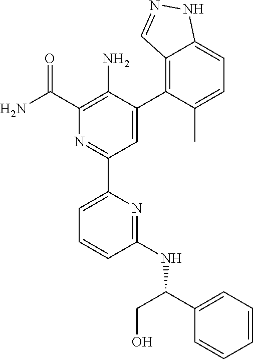 [SMILES: Cc1ccc2[nH]ncc2c1-c1cc(-c2cccc(N[C@@H](CO)c3ccccc3)n2)nc(C(N)=O)c1N] | 275 |
| 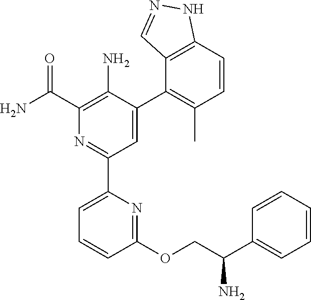 [SMILES: Cc1ccc2[nH]ncc2c1-c1cc(-c2cccc(OC[C@H](N)c3ccccc3)n2)nc(C(N)=O)c1N] | 276 |
| 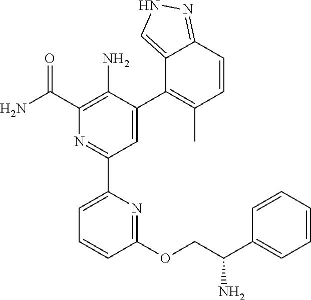 [SMILES: Cc1ccc2n[nH]cc2c1-c1cc(-c2cccc(OC[C@@H](N)c3ccccc3)n2)nc(C(N)=O)c1N] | 277 |
| 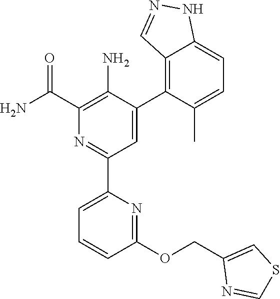 [SMILES: Cc1ccc2[nH]ncc2c1-c1cc(-c2cccc(OCc3cscn3)n2)nc(C(N)=O)c1N] | 278 |
| 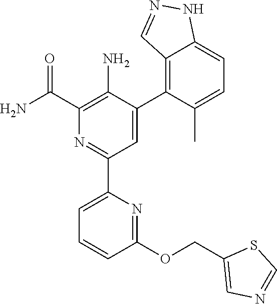 [SMILES: Cc1ccc2[nH]ncc2c1-c1cc(-c2cccc(OCc3cncs3)n2)nc(C(N)=O)c1N] | 279 |
| 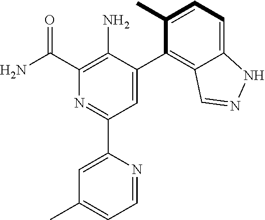 | 280 |
| 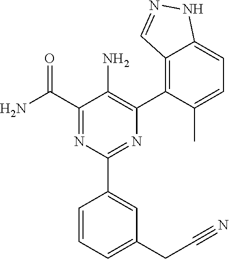 [SMILES: Cc1ccc2[nH]ncc2c1-c1nc(-c2cccc(CC#N)c2)nc(C(N)=O)c1N] | 281 |
| 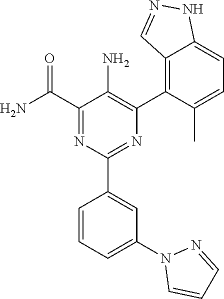 [SMILES: Cc1ccc2[nH]ncc2c1-c1nc(-c2cccc(-n3cccn3)c2)nc(C(N)=O)c1N] | 282 |
| 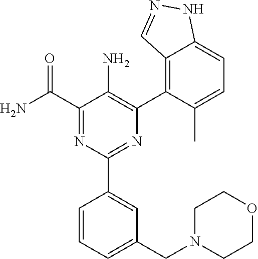 [SMILES: Cc1ccc2[nH]ncc2c1-c1nc(-c2cccc(CN3CCOCC3)c2)nc(C(N)=O)c1N] | 283 |
| 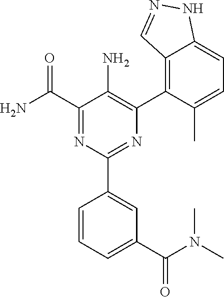 [SMILES: Cc1ccc2[nH]ncc2c1-c1nc(-c2cccc(C(=O)N(C)C)c2)nc(C(N)=O)c1N] | 284 |
| 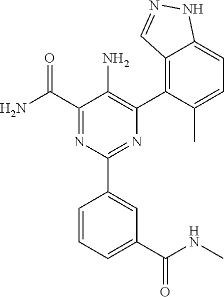 [SMILES: CNC(=O)c1cccc(-c2nc(C(N)=O)c(N)c(-c3c(C)ccc4[nH]ncc34)n2)c1] | 285 |
| 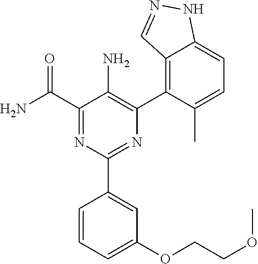 [SMILES: COCCOc1cccc(-c2nc(C(N)=O)c(N)c(-c3c(C)ccc4[nH]ncc34)n2)c1] | 286 |
| 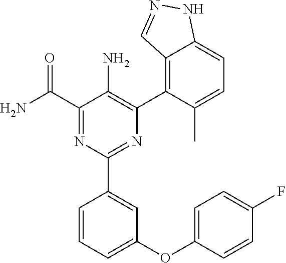 [SMILES: Cc1ccc2[nH]ncc2c1-c1nc(-c2cccc(Oc3ccc(F)cc3)c2)nc(C(N)=O)c1N] | 287 |
| 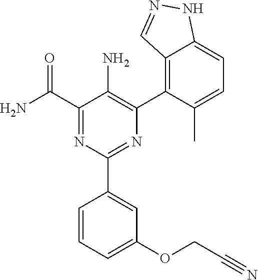 [SMILES: Cc1ccc2[nH]ncc2c1-c1nc(-c2cccc(OCC#N)c2)nc(C(N)=O)c1N] | 288 |
| 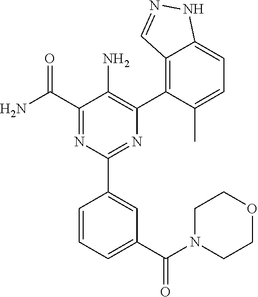 [SMILES: Cc1ccc2[nH]ncc2c1-c1nc(-c2cccc(C(=O)N3CCOCC3)c2)nc(C(N)=O)c1N] | 289 |
| 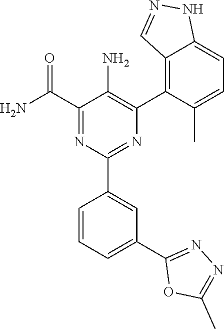 [SMILES: Cc1nnc(-c2cccc(-c3nc(C(N)=O)c(N)c(-c4c(C)ccc5[nH]ncc45)n3)c2)o1] | 290 |
| 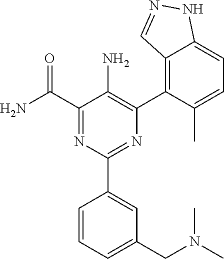 [SMILES: Cc1ccc2[nH]ncc2c1-c1nc(-c2cccc(CN(C)C)c2)nc(C(N)=O)c1N] | 291 |
| 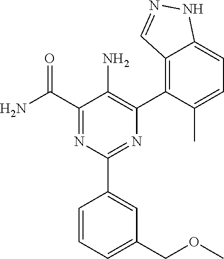 [SMILES: COCc1cccc(-c2nc(C(N)=O)c(N)c(-c3c(C)ccc4[nH]ncc34)n2)c1] | 292 |
| 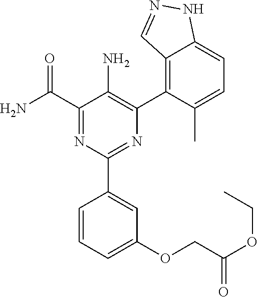 [SMILES: CCOC(=O)COc1cccc(-c2nc(C(N)=O)c(N)c(-c3c(C)ccc4[nH]ncc34)n2)c1] | 293 |
| 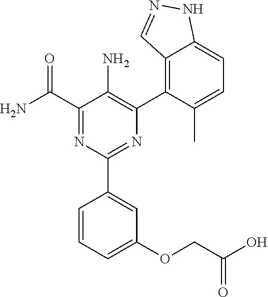 [SMILES: Cc1ccc2[nH]ncc2c1-c1nc(-c2cccc(OCC(=O)O)c2)nc(C(N)=O)c1N] | 294 |
| 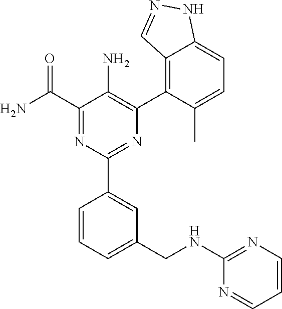 [SMILES: Cc1ccc2[nH]ncc2c1-c1nc(-c2cccc(CNc3ncccn3)c2)nc(C(N)=O)c1N] | 295 |
| 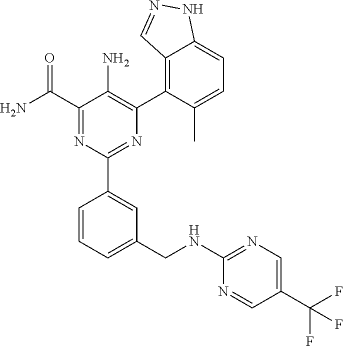 [SMILES: Cc1ccc2[nH]ncc2c1-c1nc(-c2cccc(CNc3ncc(C(F)(F)F)cn3)c2)nc(C(N)=O)c1N] | 296 |
| 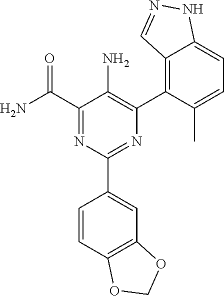 [SMILES: Cc1ccc2[nH]ncc2c1-c1nc(-c2ccc3c(c2)OCO3)nc(C(N)=O)c1N] | 297 |
| 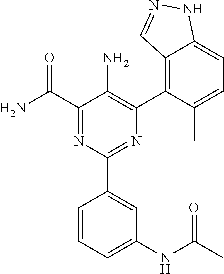 [SMILES: CC(=O)Nc1cccc(-c2nc(C(N)=O)c(N)c(-c3c(C)ccc4[nH]ncc34)n2)c1] | 298 |
| 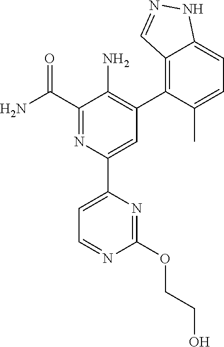 [SMILES: Cc1ccc2[nH]ncc2c1-c1cc(-c2ccnc(OCCO)n2)nc(C(N)=O)c1N] | 299 |
| 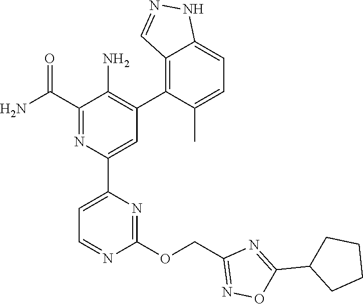 [SMILES: Cc1ccc2[nH]ncc2c1-c1cc(-c2ccnc(OCc3noc(C4CCCC4)n3)n2)nc(C(N)=O)c1N] | 300 |
| 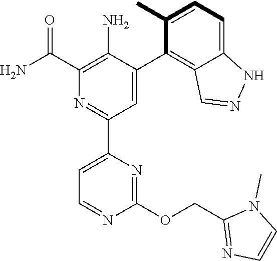 | 301 |
| 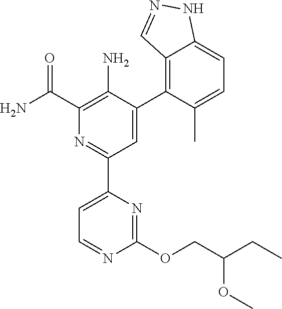 [SMILES: CCC(COc1nccc(-c2cc(-c3c(C)ccc4[nH]ncc34)c(N)c(C(N)=O)n2)n1)OC] | 302 |
| 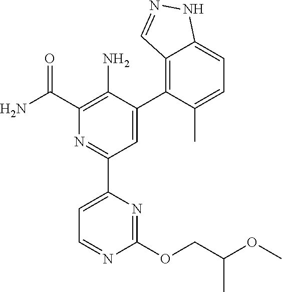 [SMILES: COC(C)COc1nccc(-c2cc(-c3c(C)ccc4[nH]ncc34)c(N)c(C(N)=O)n2)n1] | 303 |
| 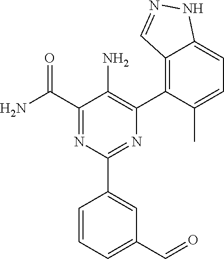 [SMILES: Cc1ccc2[nH]ncc2c1-c1nc(-c2cccc(C=O)c2)nc(C(N)=O)c1N] | 304 |
| 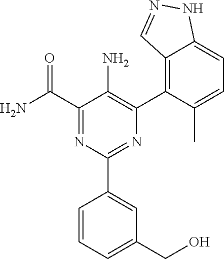 [SMILES: Cc1ccc2[nH]ncc2c1-c1nc(-c2cccc(CO)c2)nc(C(N)=O)c1N] | 305 |
| 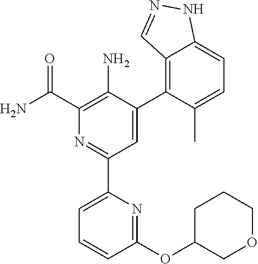 [SMILES: Cc1ccc2[nH]ncc2c1-c1cc(-c2cccc(OC3CCCOC3)n2)nc(C(N)=O)c1N] | 306 |
| 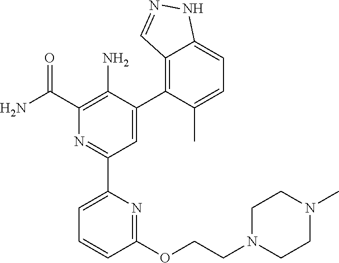 [SMILES: Cc1ccc2[nH]ncc2c1-c1cc(-c2cccc(OCCN3CCN(C)CC3)n2)nc(C(N)=O)c1N] | 307 |
| 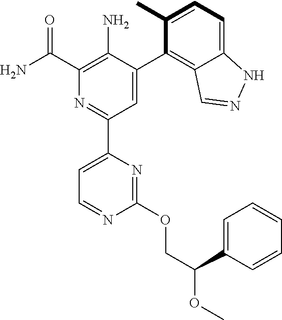 | 308 |
| 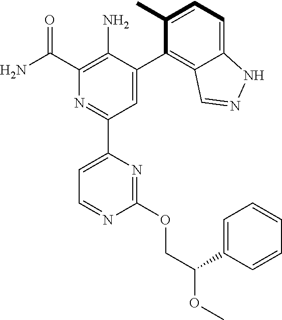 | 309 |
| 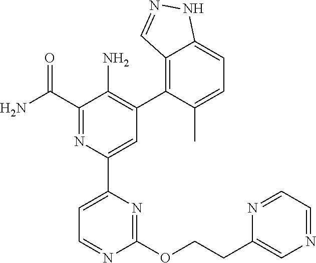 [SMILES: Cc1ccc2[nH]ncc2c1-c1cc(-c2ccnc(OCCc3cnccn3)n2)nc(C(N)=O)c1N] | 310 |
| 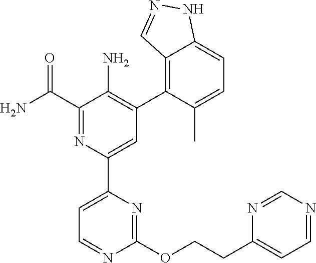 [SMILES: Cc1ccc2[nH]ncc2c1-c1cc(-c2ccnc(OCCc3ccncn3)n2)nc(C(N)=O)c1N] | 311 |
| 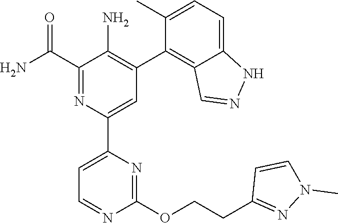 [SMILES: Cc1ccc2[nH]ncc2c1-c1cc(-c2ccnc(OCCc3ccn(C)n3)n2)nc(C(N)=O)c1N] | 312 |
| 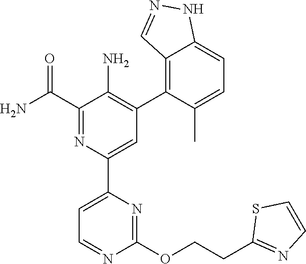 [SMILES: Cc1ccc2[nH]ncc2c1-c1cc(-c2ccnc(OCCc3nccs3)n2)nc(C(N)=O)c1N] | 313 |
| 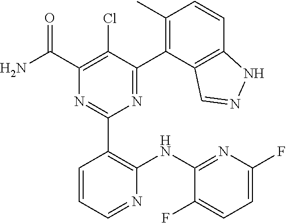 [SMILES: Cc1ccc2[nH]ncc2c1-c1nc(-c2cccnc2Nc2nc(F)ccc2F)nc(C(N)=O)c1Cl] | 314 |
| 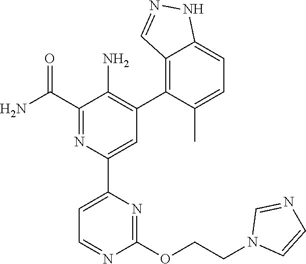 [SMILES: Cc1ccc2[nH]ncc2c1-c1cc(-c2ccnc(OCCn3ccnc3)n2)nc(C(N)=O)c1N] | 315 |
| 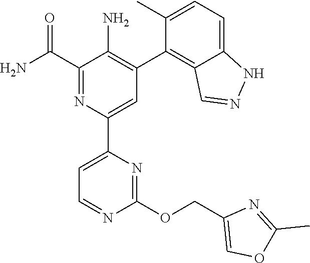 [SMILES: Cc1nc(COc2nccc(-c3cc(-c4c(C)ccc5[nH]ncc45)c(N)c(C(N)=O)n3)n2)co1] | 316 |
| 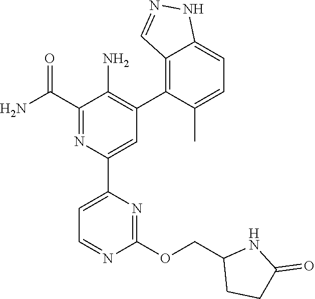 [SMILES: Cc1ccc2[nH]ncc2c1-c1cc(-c2ccnc(OCC3CCC(=O)N3)n2)nc(C(N)=O)c1N] | 317 |
| 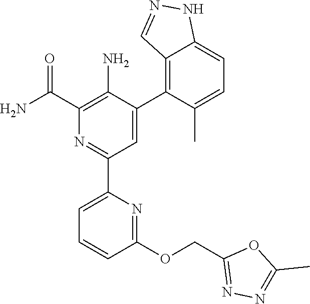 [SMILES: Cc1nnc(COc2cccc(-c3cc(-c4c(C)ccc5[nH]ncc45)c(N)c(C(N)=O)n3)n2)o1] | 318 |
| 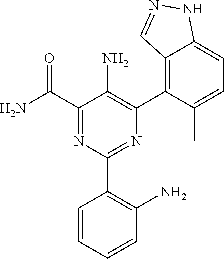 [SMILES: Cc1ccc2[nH]ncc2c1-c1nc(-c2ccccc2N)nc(C(N)=O)c1N] | 319 |
| 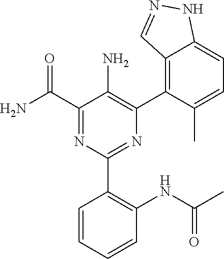 [SMILES: CC(=O)Nc1ccccc1-c1nc(C(N)=O)c(N)c(-c2c(C)ccc3[nH]ncc23)n1] | 320 |
| 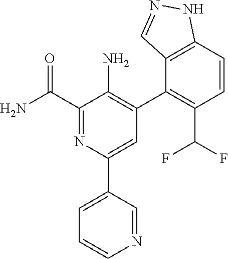 [SMILES: NC(=O)c1nc(-c2cccnc2)cc(-c2c(C(F)F)ccc3[nH]ncc23)c1N] | 321 |
| 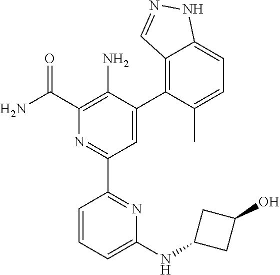 [SMILES: Cc1ccc2[nH]ncc2c1-c1cc(-c2cccc(N[C@H]3C[C@H](O)C3)n2)nc(C(N)=O)c1N] | 322 |
| 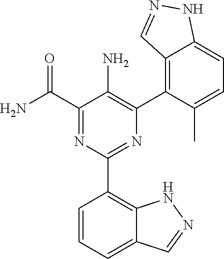 [SMILES: Cc1ccc2[nH]ncc2c1-c1nc(-c2cccc3cn[nH]c23)nc(C(N)=O)c1N] | 323 |
| 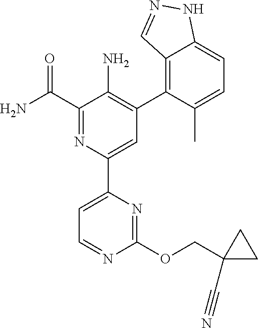 [SMILES: Cc1ccc2[nH]ncc2c1-c1cc(-c2ccnc(OCC3(C#N)CC3)n2)nc(C(N)=O)c1N] | 324 |
| 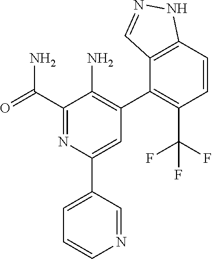 [SMILES: NC(=O)c1nc(-c2cccnc2)cc(-c2c(C(F)(F)F)ccc3[nH]ncc23)c1N] | 325 |
| 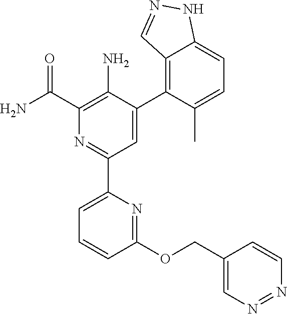 [SMILES: Cc1ccc2[nH]ncc2c1-c1cc(-c2cccc(OCc3ccnnc3)n2)nc(C(N)=O)c1N] | 326 |
| 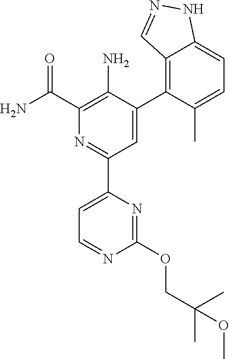 [SMILES: COC(C)(C)COc1nccc(-c2cc(-c3c(C)ccc4[nH]ncc34)c(N)c(C(N)=O)n2)n1] | 327 |
| 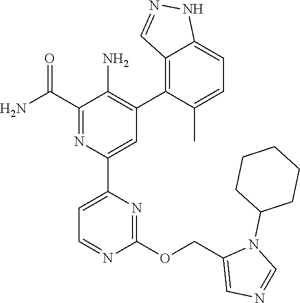 [SMILES: Cc1ccc2[nH]ncc2c1-c1cc(-c2ccnc(OCc3cncn3C3CCCCC3)n2)nc(C(N)=O)c1N] | 328 |
| 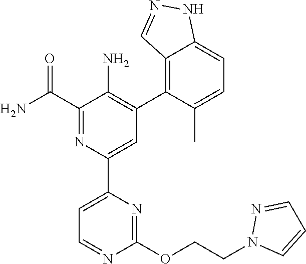 [SMILES: Cc1ccc2[nH]ncc2c1-c1cc(-c2ccnc(OCCn3cccn3)n2)nc(C(N)=O)c1N] | 329 |
| 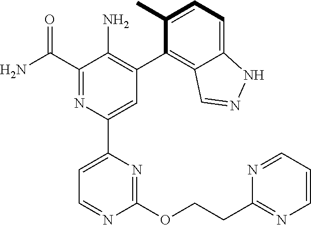 | 330 |
| 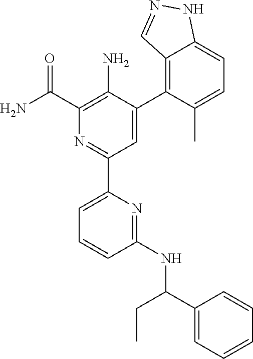 [SMILES: CCC(Nc1cccc(-c2cc(-c3c(C)ccc4[nH]ncc34)c(N)c(C(N)=O)n2)n1)c1ccccc1] | 331 |
| 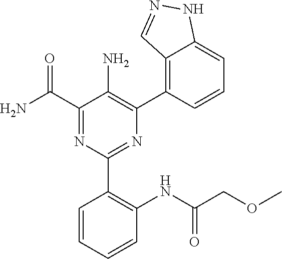 [SMILES: COCC(=O)Nc1ccccc1-c1nc(C(N)=O)c(N)c(-c2cccc3[nH]ncc23)n1] | 332 |
| 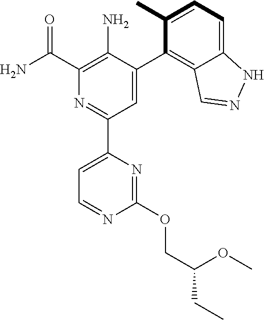 | 333 |
| 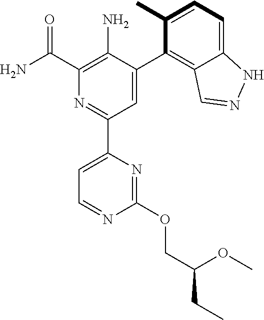 | 334 |
| 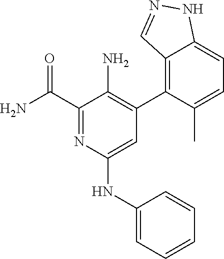 [SMILES: Cc1ccc2[nH]ncc2c1-c1cc(Nc2ccccc2)nc(C(N)=O)c1N] | 335 |
| 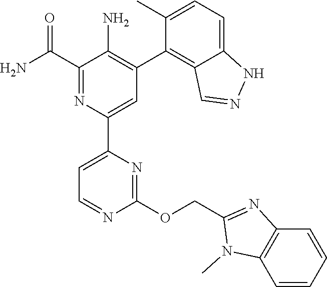 [SMILES: Cc1ccc2[nH]ncc2c1-c1cc(-c2ccnc(OCc3nc4ccccc4n3C)n2)nc(C(N)=O)c1N] | 336 |
| 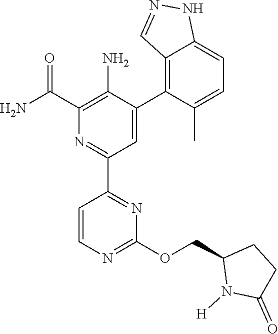 [SMILES: Cc1ccc2[nH]ncc2c1-c1cc(-c2ccnc(OC[C@H]3CCC(=O)N3)n2)nc(C(N)=O)c1N] | 337 |
| 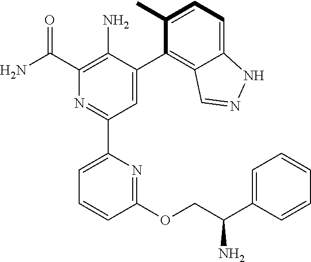 | 338 |
| 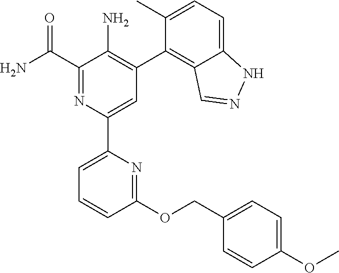 [SMILES: COc1ccc(COc2cccc(-c3cc(-c4c(C)ccc5[nH]ncc45)c(N)c(C(N)=O)n3)n2)cc1] | 339 |
| 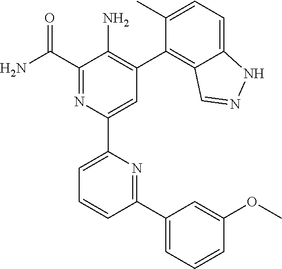 [SMILES: COc1cccc(-c2cccc(-c3cc(-c4c(C)ccc5[nH]ncc45)c(N)c(C(N)=O)n3)n2)c1] | 340 |
| 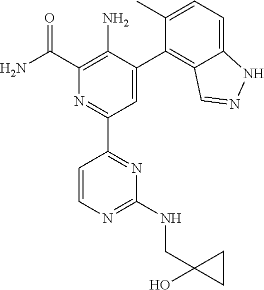 [SMILES: Cc1ccc2[nH]ncc2c1-c1cc(-c2ccnc(NCC3(O)CC3)n2)nc(C(N)=O)c1N] | 341 |
| 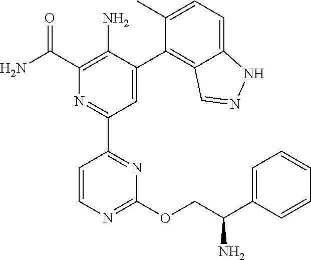 [SMILES: Cc1ccc2[nH]ncc2c1-c1cc(-c2ccnc(OC[C@H](N)c3ccccc3)n2)nc(C(N)=O)c1N] | 342 |
| 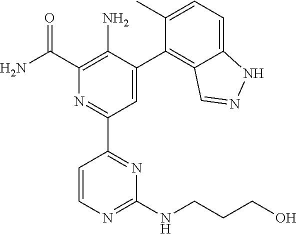 [SMILES: Cc1ccc2[nH]ncc2c1-c1cc(-c2ccnc(NCCCO)n2)nc(C(N)=O)c1N] | 343 |
| 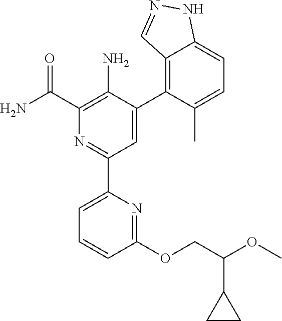 [SMILES: COC(COc1cccc(-c2cc(-c3c(C)ccc4[nH]ncc34)c(N)c(C(N)=O)n2)n1)C1CC1] | 344 |
| 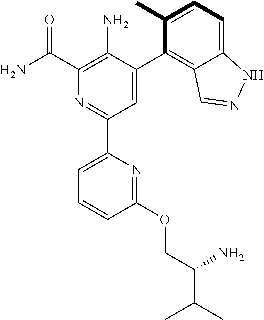 | 345 |
| 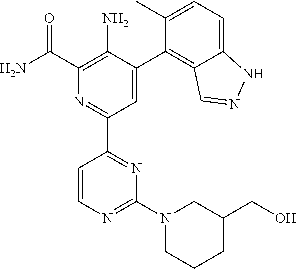 [SMILES: Cc1ccc2[nH]ncc2c1-c1cc(-c2ccnc(N3CCCC(CO)C3)n2)nc(C(N)=O)c1N] | 346 |
| 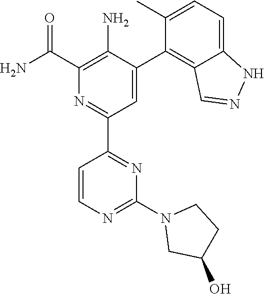 [SMILES: Cc1ccc2[nH]ncc2c1-c1cc(-c2ccnc(N3CC[C@@H](O)C3)n2)nc(C(N)=O)c1N] | 347 |
| 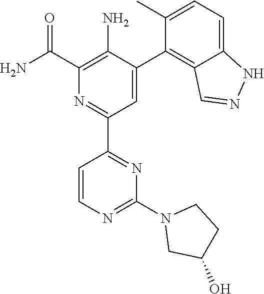 [SMILES: Cc1ccc2[nH]ncc2c1-c1cc(-c2ccnc(N3CC[C@H](O)C3)n2)nc(C(N)=O)c1N] | 348 |
| 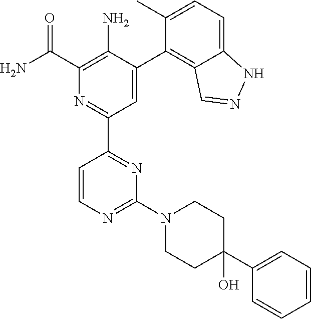 [SMILES: Cc1ccc2[nH]ncc2c1-c1cc(-c2ccnc(N3CCC(O)(c4ccccc4)CC3)n2)nc(C(N)=O)c1N] | 349 |
| 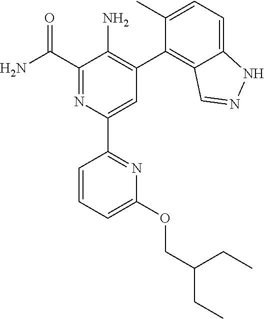 [SMILES: CCC(CC)COc1cccc(-c2cc(-c3c(C)ccc4[nH]ncc34)c(N)c(C(N)=O)n2)n1] | 350 |
| 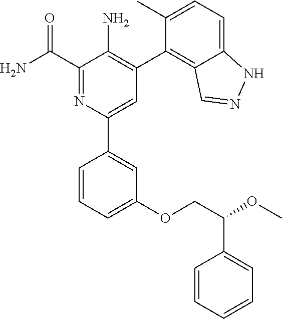 [SMILES: CO[C@@H](COc1cccc(-c2cc(-c3c(C)ccc4[nH]ncc34)c(N)c(C(N)=O)n2)c1)c1ccccc1] | 351 |
| 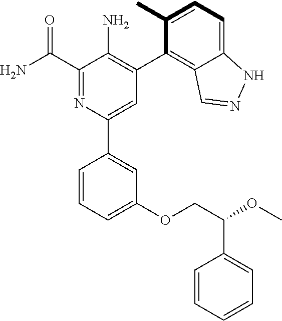 | 352 |
| 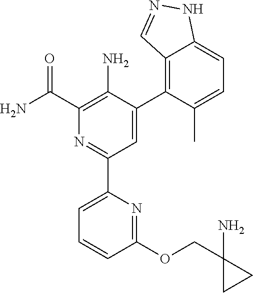 [SMILES: Cc1ccc2[nH]ncc2c1-c1cc(-c2cccc(OCC3(N)CC3)n2)nc(C(N)=O)c1N] | 353 |
| 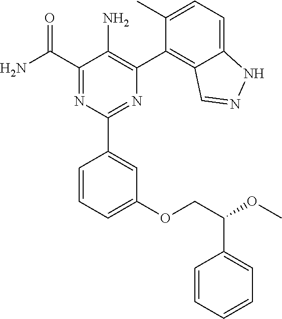 [SMILES: CO[C@@H](COc1cccc(-c2nc(C(N)=O)c(N)c(-c3c(C)ccc4[nH]ncc34)n2)c1)c1ccccc1] | 354 |
| 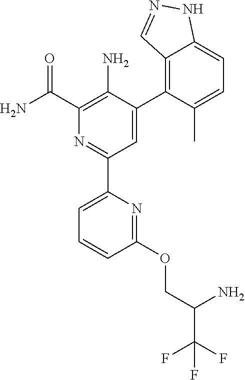 [SMILES: Cc1ccc2[nH]ncc2c1-c1cc(-c2cccc(OCC(N)C(F)(F)F)n2)nc(C(N)=O)c1N] | 355 |
| 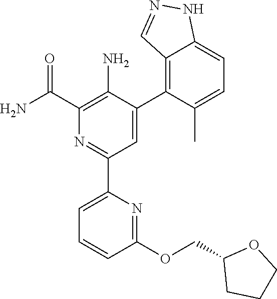 [SMILES: Cc1ccc2[nH]ncc2c1-c1cc(-c2cccc(OC[C@H]3CCCO3)n2)nc(C(N)=O)c1N] | 356 |
| 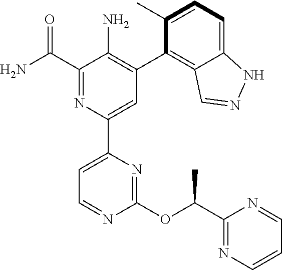 | 357 |
| 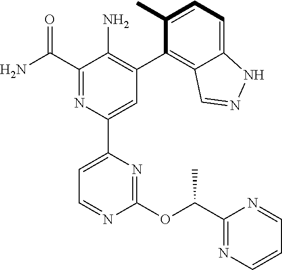 | 358 |
| 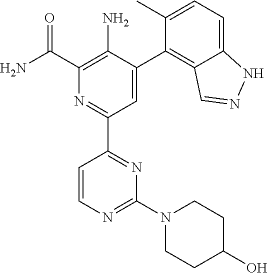 [SMILES: Cc1ccc2[nH]ncc2c1-c1cc(-c2ccnc(N3CCC(O)CC3)n2)nc(C(N)=O)c1N] | 359 |
| 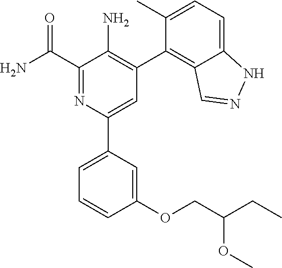 [SMILES: CCC(COc1cccc(-c2cc(-c3c(C)ccc4[nH]ncc34)c(N)c(C(N)=O)n2)c1)OC] | 360 |
| 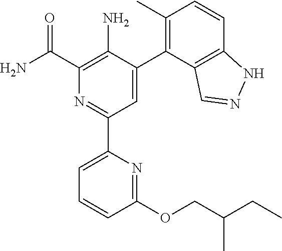 [SMILES: CCC(C)COc1cccc(-c2cc(-c3c(C)ccc4[nH]ncc34)c(N)c(C(N)=O)n2)n1] | 361 |
| 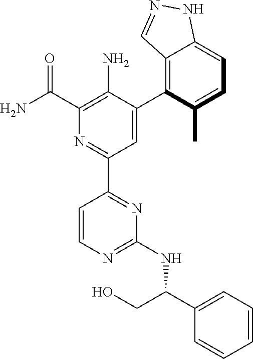 | 362 |
| 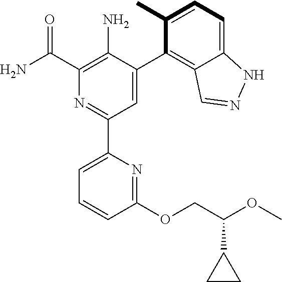 | 363 |
| 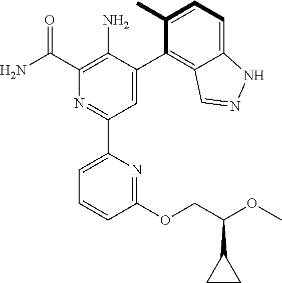 | 364 |
| 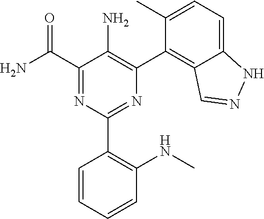 [SMILES: CNc1ccccc1-c1nc(C(N)=O)c(N)c(-c2c(C)ccc3[nH]ncc23)n1] | 365 |
| 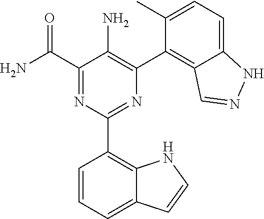 [SMILES: Cc1ccc2[nH]ncc2c1-c1nc(-c2cccc3cc[nH]c23)nc(C(N)=O)c1N] | 366 |
| 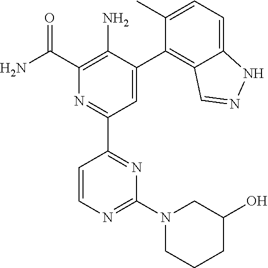 [SMILES: Cc1ccc2[nH]ncc2c1-c1cc(-c2ccnc(N3CCCC(O)C3)n2)nc(C(N)=O)c1N] | 367 |
| 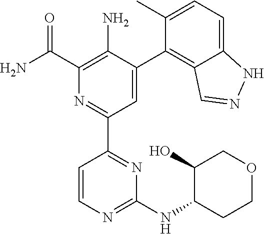 [SMILES: Cc1ccc2[nH]ncc2c1-c1cc(-c2ccnc(N[C@H]3CCOC[C@@H]3O)n2)nc(C(N)=O)c1N] | 368 |
| 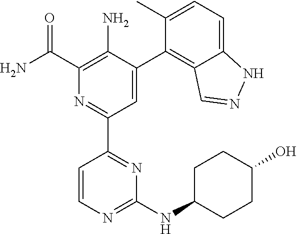 [SMILES: Cc1ccc2[nH]ncc2c1-c1cc(-c2ccnc(N[C@H]3CC[C@H](O)CC3)n2)nc(C(N)=O)c1N] | 369 |
| 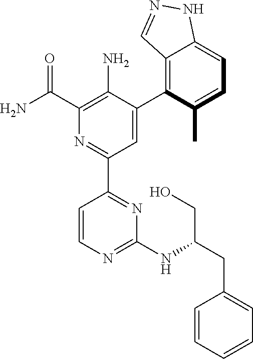 | 370 |
| 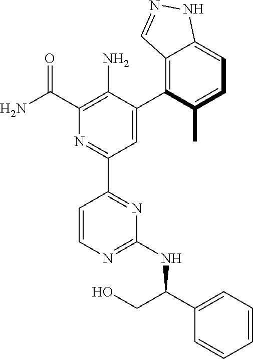 | 371 |
| 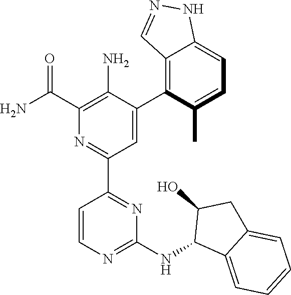 | 372 |
| 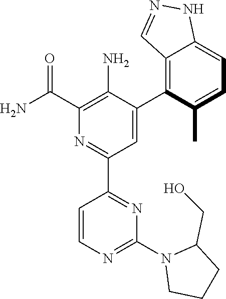 | 373 |
| 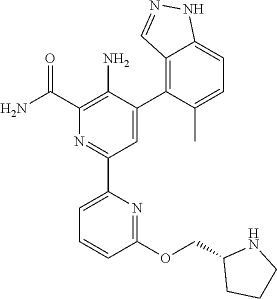 [SMILES: Cc1ccc2[nH]ncc2c1-c1cc(-c2cccc(OC[C@H]3CCCN3)n2)nc(C(N)=O)c1N] | 374 |
| 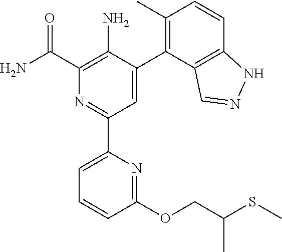 [SMILES: CSC(C)COc1cccc(-c2cc(-c3c(C)ccc4[nH]ncc34)c(N)c(C(N)=O)n2)n1] | 375 |
| 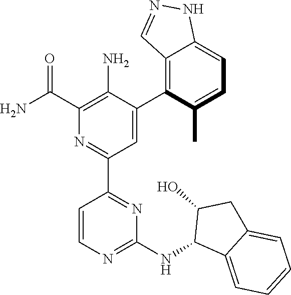 | 376 |
| 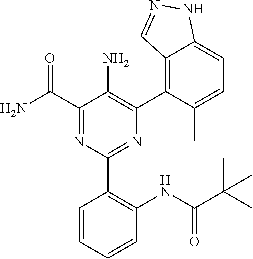 [SMILES: Cc1ccc2[nH]ncc2c1-c1nc(-c2ccccc2NC(=O)C(C)(C)C)nc(C(N)=O)c1N] | 377 |
| 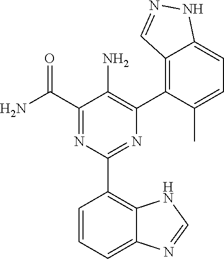 [SMILES: Cc1ccc2[nH]ncc2c1-c1nc(-c2cccc3nc[nH]c23)nc(C(N)=O)c1N] | 378 |
| 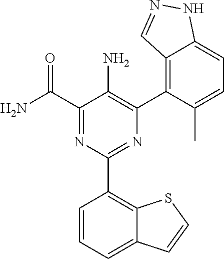 [SMILES: Cc1ccc2[nH]ncc2c1-c1nc(-c2cccc3ccsc23)nc(C(N)=O)c1N] | 379 |
| 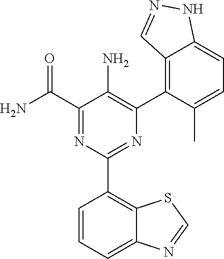 [SMILES: Cc1ccc2[nH]ncc2c1-c1nc(-c2cccc3ncsc23)nc(C(N)=O)c1N] | 380 |
| 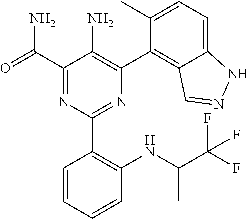 [SMILES: Cc1ccc2[nH]ncc2c1-c1nc(-c2ccccc2NC(C)C(F)(F)F)nc(C(N)=O)c1N] | 381 |
| 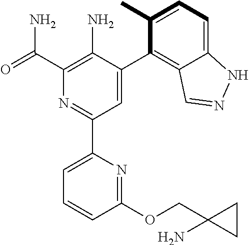 | 382 |
| 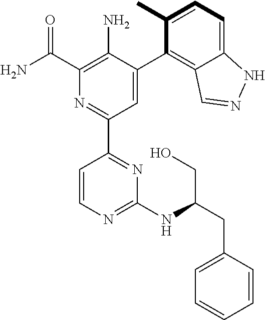 | 383 |
| 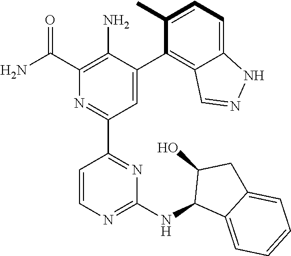 | 384 |
| 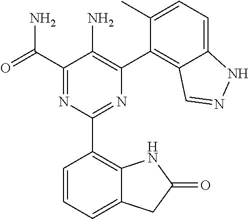 [SMILES: Cc1ccc2[nH]ncc2c1-c1nc(-c2cccc3c2NC(=O)C3)nc(C(N)=O)c1N] | 385 |
| 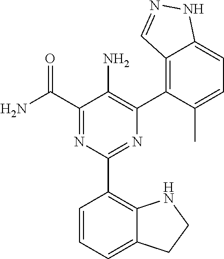 [SMILES: Cc1ccc2[nH]ncc2c1-c1nc(-c2cccc3c2NCC3)nc(C(N)=O)c1N] | 386 |
| 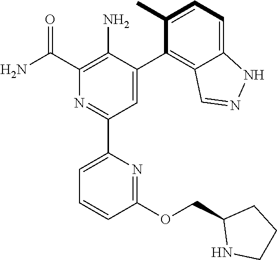 | 387 |
| 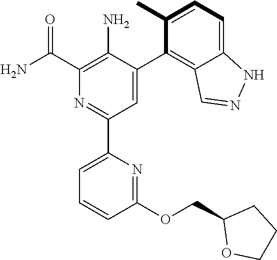 | 388 |
| 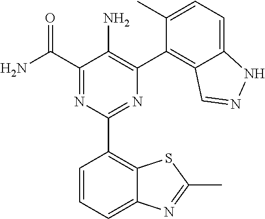 [SMILES: Cc1nc2cccc(-c3nc(C(N)=O)c(N)c(-c4c(C)ccc5[nH]ncc45)n3)c2s1] | 389 |
| 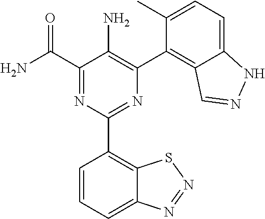 [SMILES: Cc1ccc2[nH]ncc2c1-c1nc(-c2cccc3nnsc23)nc(C(N)=O)c1N] | 390 |
| 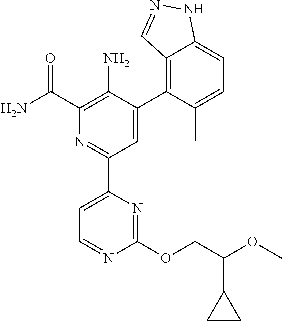 [SMILES: COC(COc1nccc(-c2cc(-c3c(C)ccc4[nH]ncc34)c(N)c(C(N)=O)n2)n1)C1CC1] | 391 |
| 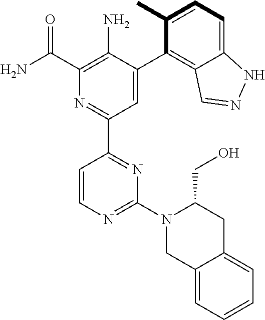 | 392 |
| 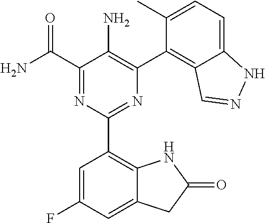 [SMILES: Cc1ccc2[nH]ncc2c1-c1nc(-c2cc(F)cc3c2NC(=O)C3)nc(C(N)=O)c1N] | 393 |
| 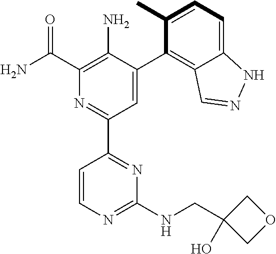 | 394 |
| 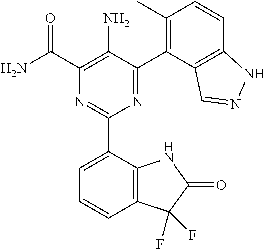 [SMILES: Cc1ccc2[nH]ncc2c1-c1nc(-c2cccc3c2NC(=O)C3(F)F)nc(C(N)=O)c1N] | 395 |
| 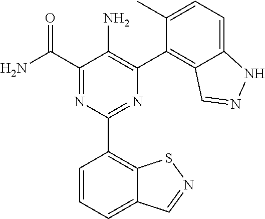 [SMILES: Cc1ccc2[nH]ncc2c1-c1nc(-c2cccc3cnsc23)nc(C(N)=O)c1N] | 396 |
| 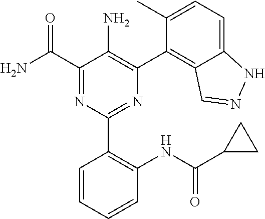 [SMILES: Cc1ccc2[nH]ncc2c1-c1nc(-c2ccccc2NC(=O)C2CC2)nc(C(N)=O)c1N] | 397 |
| 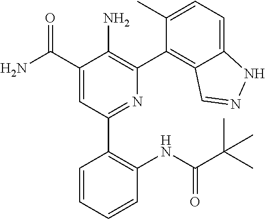 [SMILES: Cc1ccc2[nH]ncc2c1-c1nc(-c2ccccc2NC(=O)C(C)(C)C)cc(C(N)=O)c1N] | 398 |
| 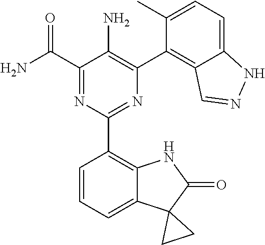 [SMILES: Cc1ccc2[nH]ncc2c1-c1nc(-c2cccc3c2NC(=O)C32CC2)nc(C(N)=O)c1N] | 399 |
| 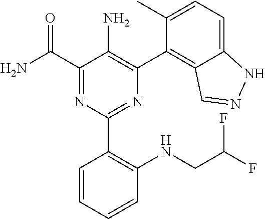 [SMILES: Cc1ccc2[nH]ncc2c1-c1nc(-c2ccccc2NCC(F)F)nc(C(N)=O)c1N] | 400 |
| 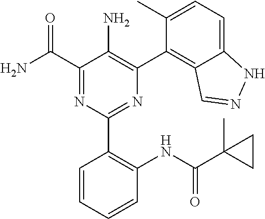 [SMILES: Cc1ccc2[nH]ncc2c1-c1nc(-c2ccccc2NC(=O)C2(C)CC2)nc(C(N)=O)c1N] | 401 |
| 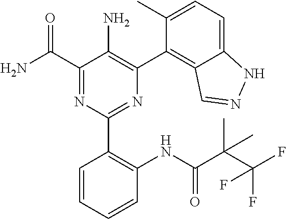 [SMILES: Cc1ccc2[nH]ncc2c1-c1nc(-c2ccccc2NC(=O)C(C)(C)C(F)(F)F)nc(C(N)=O)c1N] | 402 |
| 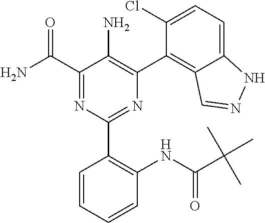 [SMILES: CC(C)(C)C(=O)Nc1ccccc1-c1nc(C(N)=O)c(N)c(-c2c(Cl)ccc3[nH]ncc23)n1] | 403 |
| 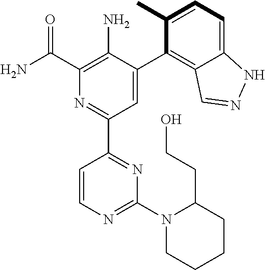 | 404 |
| 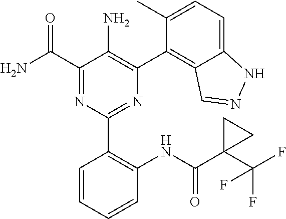 [SMILES: Cc1ccc2[nH]ncc2c1-c1nc(-c2ccccc2NC(=O)C2(C(F)(F)F)CC2)nc(C(N)=O)c1N] | 405 |
| 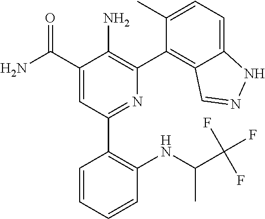 [SMILES: Cc1ccc2[nH]ncc2c1-c1nc(-c2ccccc2NC(C)C(F)(F)F)cc(C(N)=O)c1N] | 406 |
| 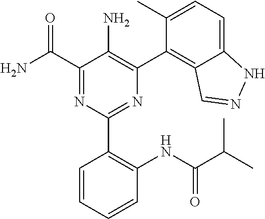 [SMILES: Cc1ccc2[nH]ncc2c1-c1nc(-c2ccccc2NC(=O)C(C)C)nc(C(N)=O)c1N] | 407 |
| 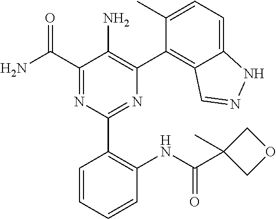 [SMILES: Cc1ccc2[nH]ncc2c1-c1nc(-c2ccccc2NC(=O)C2(C)COC2)nc(C(N)=O)c1N] | 408 |
| 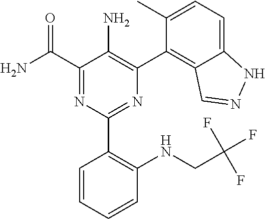 [SMILES: Cc1ccc2[nH]ncc2c1-c1nc(-c2ccccc2NCC(F)(F)F)nc(C(N)=O)c1N] | 409 |
| 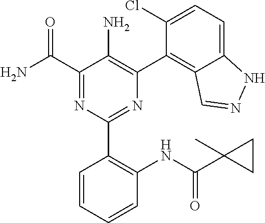 [SMILES: CC1(C(=O)Nc2ccccc2-c2nc(C(N)=O)c(N)c(-c3c(Cl)ccc4[nH]ncc34)n2)CC1] | 410 |
| 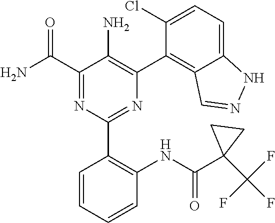 [SMILES: NC(=O)c1nc(-c2ccccc2NC(=O)C2(C(F)(F)F)CC2)nc(-c2c(Cl)ccc3[nH]ncc23)c1N] | 411 |
| 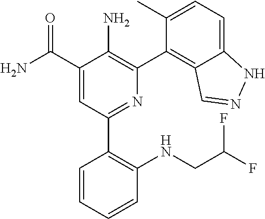 [SMILES: Cc1ccc2[nH]ncc2c1-c1nc(-c2ccccc2NCC(F)F)cc(C(N)=O)c1N] | 412 |
| 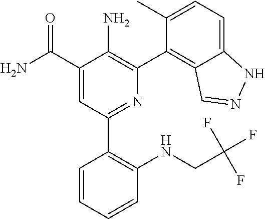 [SMILES: Cc1ccc2[nH]ncc2c1-c1nc(-c2ccccc2NCC(F)(F)F)cc(C(N)=O)c1N] | 413 |
| 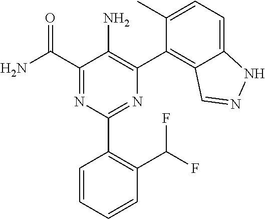 [SMILES: Cc1ccc2[nH]ncc2c1-c1nc(-c2ccccc2C(F)F)nc(C(N)=O)c1N] | 414 |
| 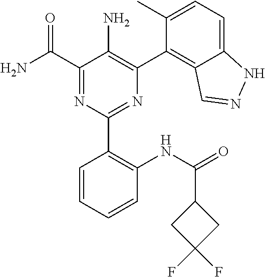 [SMILES: Cc1ccc2[nH]ncc2c1-c1nc(-c2ccccc2NC(=O)C2CC(F)(F)C2)nc(C(N)=O)c1N] | 415 |
| 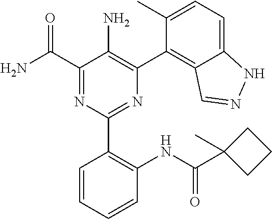 [SMILES: Cc1ccc2[nH]ncc2c1-c1nc(-c2ccccc2NC(=O)C2(C)CCC2)nc(C(N)=O)c1N] | 416 |
| 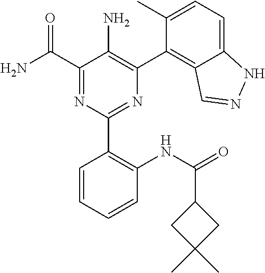 [SMILES: Cc1ccc2[nH]ncc2c1-c1nc(-c2ccccc2NC(=O)C2CC(C)(C)C2)nc(C(N)=O)c1N] | 417 |
| 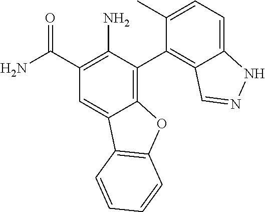 [SMILES: Cc1ccc2[nH]ncc2c1-c1c(N)c(C(N)=O)cc2c1oc1ccccc12] | 418 |
| 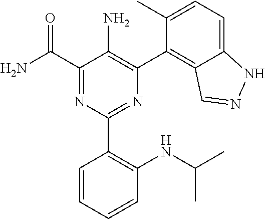 [SMILES: Cc1ccc2[nH]ncc2c1-c1nc(-c2ccccc2NC(C)C)nc(C(N)=O)c1N] | 419 |
| 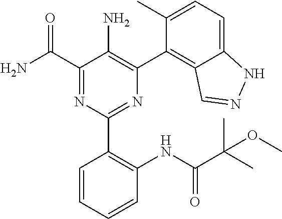 [SMILES: COC(C)(C)C(=O)Nc1ccccc1-c1nc(C(N)=O)c(N)c(-c2c(C)ccc3[nH]ncc23)n1] | 420 |
| 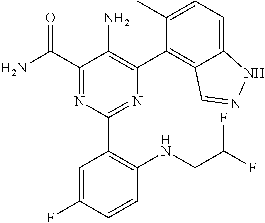 [SMILES: Cc1ccc2[nH]ncc2c1-c1nc(-c2cc(F)ccc2NCC(F)F)nc(C(N)=O)c1N] | 421 |
| 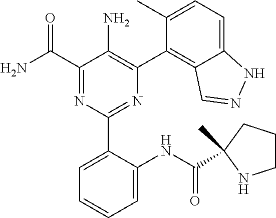 [SMILES: Cc1ccc2[nH]ncc2c1-c1nc(-c2ccccc2NC(=O)[C@]2(C)CCCN2)nc(C(N)=O)c1N] | 422 |
| 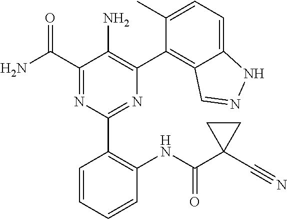 [SMILES: Cc1ccc2[nH]ncc2c1-c1nc(-c2ccccc2NC(=O)C2(C#N)CC2)nc(C(N)=O)c1N] | 423 |
| 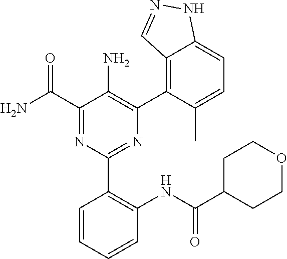 [SMILES: Cc1ccc2[nH]ncc2c1-c1nc(-c2ccccc2NC(=O)C2CCOCC2)nc(C(N)=O)c1N] | 424 |
| 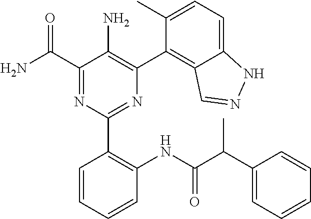 [SMILES: Cc1ccc2[nH]ncc2c1-c1nc(-c2ccccc2NC(=O)C(C)c2ccccc2)nc(C(N)=O)c1N] | 425 |
| 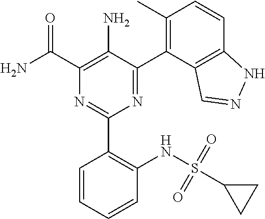 [SMILES: Cc1ccc2[nH]ncc2c1-c1nc(-c2ccccc2NS(=O)(=O)C2CC2)nc(C(N)=O)c1N] | 426 |
| 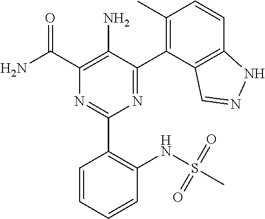 [SMILES: Cc1ccc2[nH]ncc2c1-c1nc(-c2ccccc2NS(C)(=O)=O)nc(C(N)=O)c1N] | 427 |
| 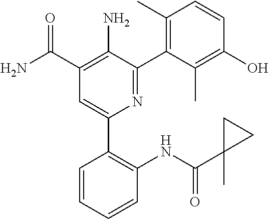 [SMILES: Cc1ccc(O)c(C)c1-c1nc(-c2ccccc2NC(=O)C2(C)CC2)cc(C(N)=O)c1N] | 428 |
| 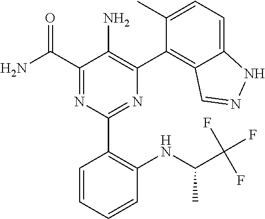 [SMILES: Cc1ccc2[nH]ncc2c1-c1nc(-c2ccccc2N[C@@H](C)C(F)(F)F)nc(C(N)=O)c1N] | 429 |
| 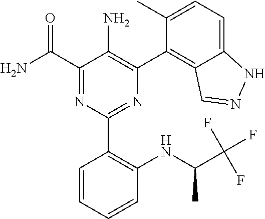 [SMILES: Cc1ccc2[nH]ncc2c1-c1nc(-c2ccccc2N[C@H](C)C(F)(F)F)nc(C(N)=O)c1N] | 430 |
| 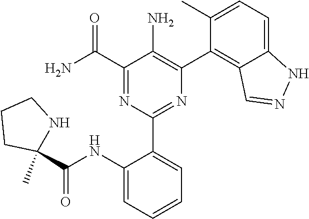 [SMILES: Cc1ccc2[nH]ncc2c1-c1nc(-c2ccccc2NC(=O)[C@@]2(C)CCCN2)nc(C(N)=O)c1N] | 431 |
| 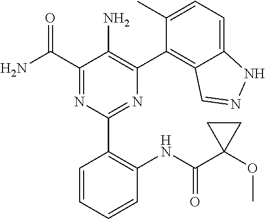 [SMILES: COC1(C(=O)Nc2ccccc2-c2nc(C(N)=O)c(N)c(-c3c(C)ccc4[nH]ncc34)n2)CC1] | 432 |
| 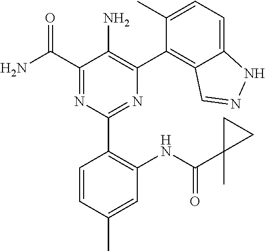 [SMILES: Cc1ccc(-c2nc(C(N)=O)c(N)c(-c3c(C)ccc4[nH]ncc34)n2)c(NC(=O)C2(C)CC2)c1] | 433 |
| 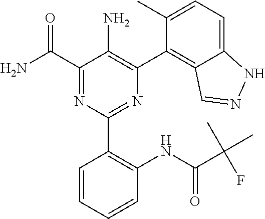 [SMILES: Cc1ccc2[nH]ncc2c1-c1nc(-c2ccccc2NC(=O)C(C)(C)F)nc(C(N)=O)c1N] | 434 |
| 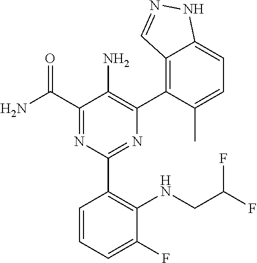 [SMILES: Cc1ccc2[nH]ncc2c1-c1nc(-c2cccc(F)c2NCC(F)F)nc(C(N)=O)c1N] | 435 |
| 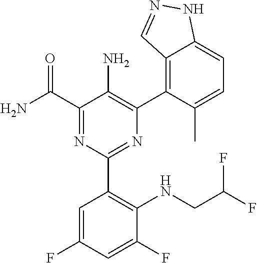 [SMILES: Cc1ccc2[nH]ncc2c1-c1nc(-c2cc(F)cc(F)c2NCC(F)F)nc(C(N)=O)c1N] | 436 |
| 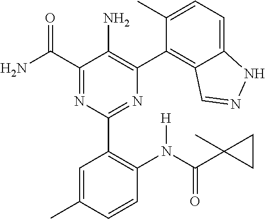 [SMILES: Cc1ccc(NC(=O)C2(C)CC2)c(-c2nc(C(N)=O)c(N)c(-c3c(C)ccc4[nH]ncc34)n2)c1] | 437 |
| 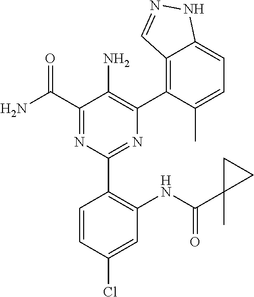 [SMILES: Cc1ccc2[nH]ncc2c1-c1nc(-c2ccc(Cl)cc2NC(=O)C2(C)CC2)nc(C(N)=O)c1N] | 438 |
| 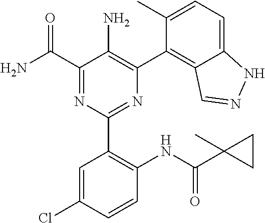 [SMILES: Cc1ccc2[nH]ncc2c1-c1nc(-c2cc(Cl)ccc2NC(=O)C2(C)CC2)nc(C(N)=O)c1N] | 439 |
| 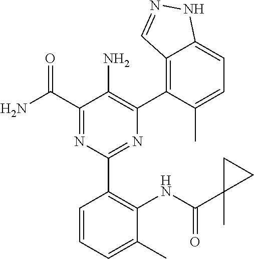 [SMILES: Cc1cccc(-c2nc(C(N)=O)c(N)c(-c3c(C)ccc4[nH]ncc34)n2)c1NC(=O)C1(C)CC1] | 440 |
| 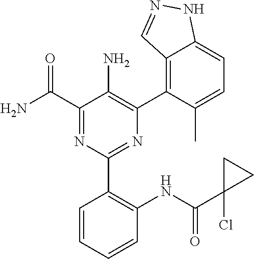 [SMILES: Cc1ccc2[nH]ncc2c1-c1nc(-c2ccccc2NC(=O)C2(Cl)CC2)nc(C(N)=O)c1N] | 441 |
| 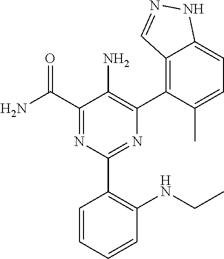 [SMILES: CCNc1ccccc1-c1nc(C(N)=O)c(N)c(-c2c(C)ccc3[nH]ncc23)n1] | 442 |
| 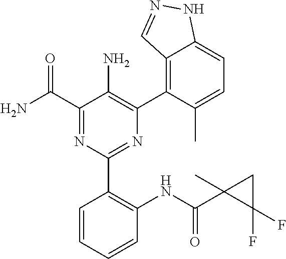 [SMILES: Cc1ccc2[nH]ncc2c1-c1nc(-c2ccccc2NC(=O)C2(C)CC2(F)F)nc(C(N)=O)c1N] | 443 |
| 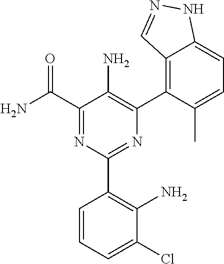 [SMILES: Cc1ccc2[nH]ncc2c1-c1nc(-c2cccc(Cl)c2N)nc(C(N)=O)c1N] | 444 |
| 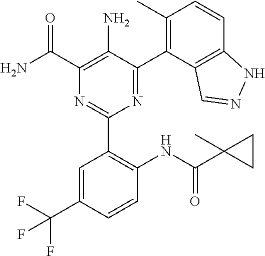 [SMILES: Cc1ccc2[nH]ncc2c1-c1nc(-c2cc(C(F)(F)F)ccc2NC(=O)C2(C)CC2)nc(C(N)=O)c1N] | 445 |
| 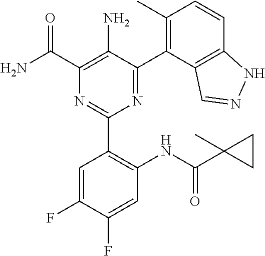 [SMILES: Cc1ccc2[nH]ncc2c1-c1nc(-c2cc(F)c(F)cc2NC(=O)C2(C)CC2)nc(C(N)=O)c1N] | 446 |
| 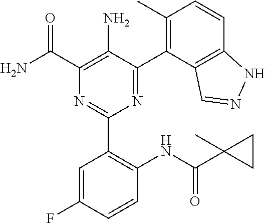 [SMILES: Cc1ccc2[nH]ncc2c1-c1nc(-c2cc(F)ccc2NC(=O)C2(C)CC2)nc(C(N)=O)c1N] | 447 |
| 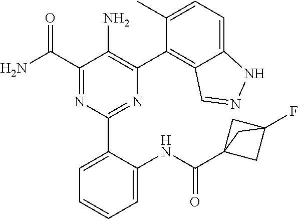 [SMILES: Cc1ccc2[nH]ncc2c1-c1nc(-c2ccccc2NC(=O)C23CC(F)(C2)C3)nc(C(N)=O)c1N] | 448 |
| 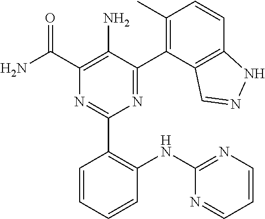 [SMILES: Cc1ccc2[nH]ncc2c1-c1nc(-c2ccccc2Nc2ncccn2)nc(C(N)=O)c1N] | 449 |
| 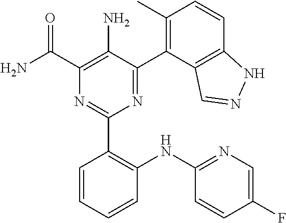 [SMILES: Cc1ccc2[nH]ncc2c1-c1nc(-c2ccccc2Nc2ccc(F)cn2)nc(C(N)=O)c1N] | 450 |
| 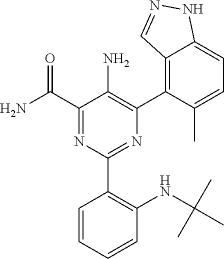 [SMILES: Cc1ccc2[nH]ncc2c1-c1nc(-c2ccccc2NC(C)(C)C)nc(C(N)=O)c1N] | 451 |
| 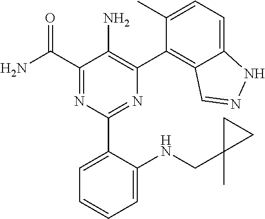 [SMILES: Cc1ccc2[nH]ncc2c1-c1nc(-c2ccccc2NCC2(C)CC2)nc(C(N)=O)c1N] | 452 |
| 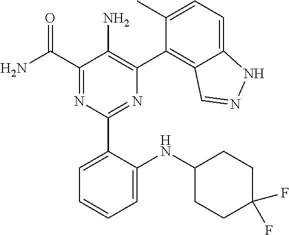 [SMILES: Cc1ccc2[nH]ncc2c1-c1nc(-c2ccccc2NC2CCC(F)(F)CC2)nc(C(N)=O)c1N] | 453 |
| 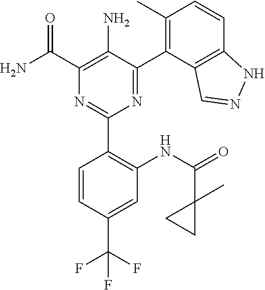 [SMILES: Cc1ccc2[nH]ncc2c1-c1nc(-c2ccc(C(F)(F)F)cc2NC(=O)C2(C)CC2)nc(C(N)=O)c1N] | 454 |
| 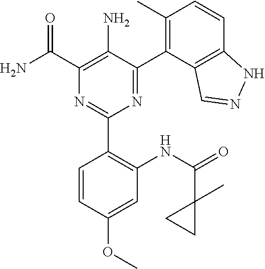 [SMILES: COc1ccc(-c2nc(C(N)=O)c(N)c(-c3c(C)ccc4[nH]ncc34)n2)c(NC(=O)C2(C)CC2)c1] | 455 |
| 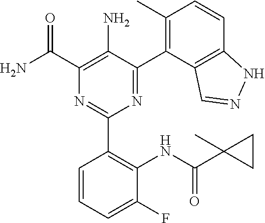 [SMILES: Cc1ccc2[nH]ncc2c1-c1nc(-c2cccc(F)c2NC(=O)C2(C)CC2)nc(C(N)=O)c1N] | 456 |
| 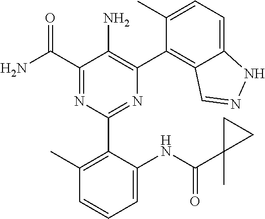 [SMILES: Cc1cccc(NC(=O)C2(C)CC2)c1-c1nc(C(N)=O)c(N)c(-c2c(C)ccc3[nH]ncc23)n1] | 457 |
| 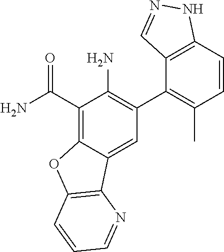 [SMILES: Cc1ccc2[nH]ncc2c1-c1cc2c(oc3cccnc32)c(C(N)=O)c1N] | 458 |
| 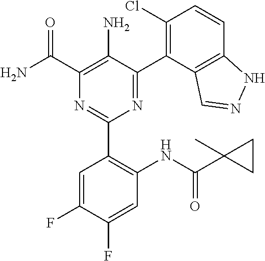 [SMILES: CC1(C(=O)Nc2cc(F)c(F)cc2-c2nc(C(N)=O)c(N)c(-c3c(Cl)ccc4[nH]ncc34)n2)CC1] | 459 |
| 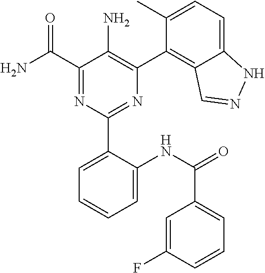 [SMILES: Cc1ccc2[nH]ncc2c1-c1nc(-c2ccccc2NC(=O)c2cccc(F)c2)nc(C(N)=O)c1N] | 460 |
| 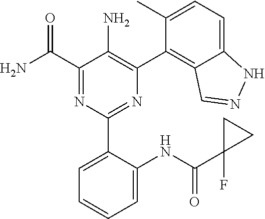 [SMILES: Cc1ccc2[nH]ncc2c1-c1nc(-c2ccccc2NC(=O)C2(F)CC2)nc(C(N)=O)c1N] | 461 |
| 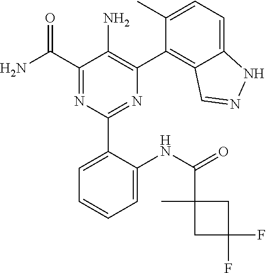 [SMILES: Cc1ccc2[nH]ncc2c1-c1nc(-c2ccccc2NC(=O)C2(C)CC(F)(F)C2)nc(C(N)=O)c1N] | 462 |
| 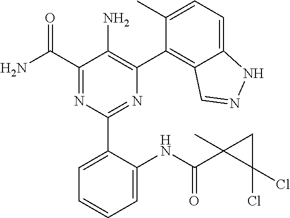 [SMILES: Cc1ccc2[nH]ncc2c1-c1nc(-c2ccccc2NC(=O)C2(C)CC2(Cl)Cl)nc(C(N)=O)c1N] | 463 |
| 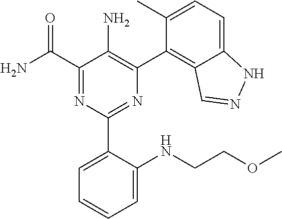 [SMILES: COCCNc1ccccc1-c1nc(C(N)=O)c(N)c(-c2c(C)ccc3[nH]ncc23)n1] | 464 |
| 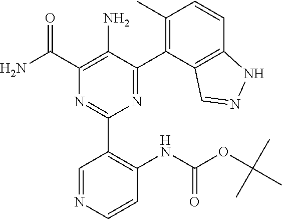 [SMILES: Cc1ccc2[nH]ncc2c1-c1nc(-c2cnccc2NC(=O)OC(C)(C)C)nc(C(N)=O)c1N] | 465 |
| 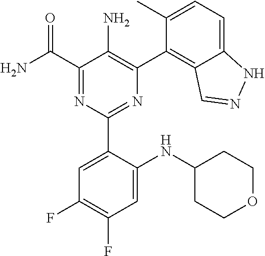 [SMILES: Cc1ccc2[nH]ncc2c1-c1nc(-c2cc(F)c(F)cc2NC2CCOCC2)nc(C(N)=O)c1N] | 466 |
| 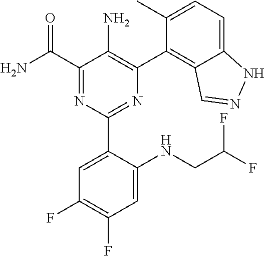 [SMILES: Cc1ccc2[nH]ncc2c1-c1nc(-c2cc(F)c(F)cc2NCC(F)F)nc(C(N)=O)c1N] | 467 |
| 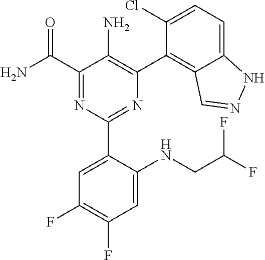 [SMILES: NC(=O)c1nc(-c2cc(F)c(F)cc2NCC(F)F)nc(-c2c(Cl)ccc3[nH]ncc23)c1N] | 468 |
| 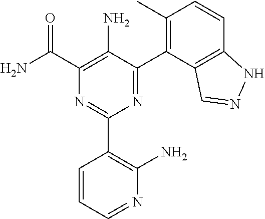 [SMILES: Cc1ccc2[nH]ncc2c1-c1nc(-c2cccnc2N)nc(C(N)=O)c1N] | 469 |
| 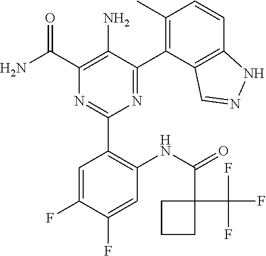 [SMILES: Cc1ccc2[nH]ncc2c1-c1nc(-c2cc(F)c(F)cc2NC(=O)C2(C(F)(F)F)CCC2)nc(C(N)=O)c1N] | 470 |
| 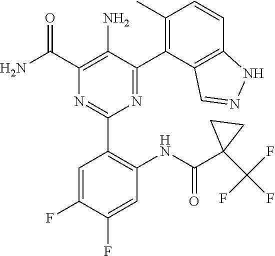 [SMILES: Cc1ccc2[nH]ncc2c1-c1nc(-c2cc(F)c(F)cc2NC(=O)C2(C(F)(F)F)CC2)nc(C(N)=O)c1N] | 471 |
| 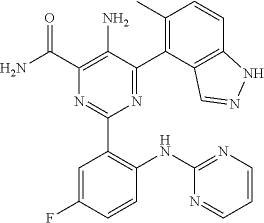 [SMILES: Cc1ccc2[nH]ncc2c1-c1nc(-c2cc(F)ccc2Nc2ncccn2)nc(C(N)=O)c1N] | 472 |
| 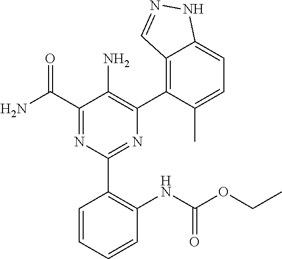 [SMILES: CCOC(=O)Nc1ccccc1-c1nc(C(N)=O)c(N)c(-c2c(C)ccc3[nH]ncc23)n1] | 473 |
| 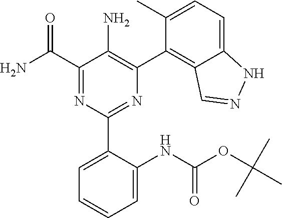 [SMILES: Cc1ccc2[nH]ncc2c1-c1nc(-c2ccccc2NC(=O)OC(C)(C)C)nc(C(N)=O)c1N] | 474 |
| 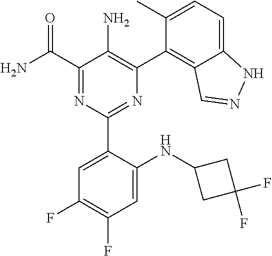 [SMILES: Cc1ccc2[nH]ncc2c1-c1nc(-c2cc(F)c(F)cc2NC2CC(F)(F)C2)nc(C(N)=O)c1N] | 475 |
| 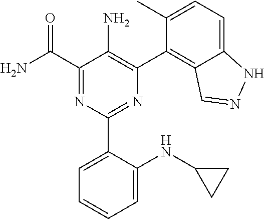 [SMILES: Cc1ccc2[nH]ncc2c1-c1nc(-c2ccccc2NC2CC2)nc(C(N)=O)c1N] | 476 |
| 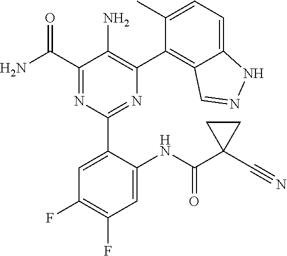 [SMILES: Cc1ccc2[nH]ncc2c1-c1nc(-c2cc(F)c(F)cc2NC(=O)C2(C#N)CC2)nc(C(N)=O)c1N] | 477 |
| 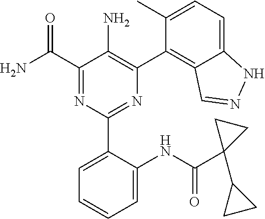 [SMILES: Cc1ccc2[nH]ncc2c1-c1nc(-c2ccccc2NC(=O)C2(C3CC3)CC2)nc(C(N)=O)c1N] | 478 |
| 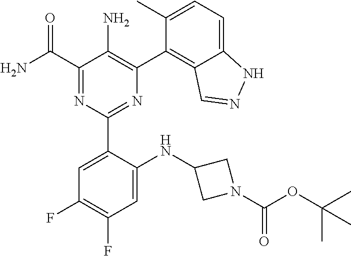 [SMILES: Cc1ccc2[nH]ncc2c1-c1nc(-c2cc(F)c(F)cc2NC2CN(C(=O)OC(C)(C)C)C2)nc(C(N)=O)c1N] | 479 |
| 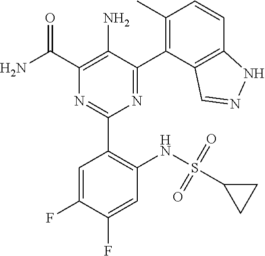 [SMILES: Cc1ccc2[nH]ncc2c1-c1nc(-c2cc(F)c(F)cc2NS(=O)(=O)C2CC2)nc(C(N)=O)c1N] | 480 |
| 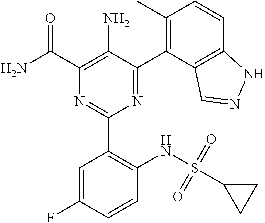 [SMILES: Cc1ccc2[nH]ncc2c1-c1nc(-c2cc(F)ccc2NS(=O)(=O)C2CC2)nc(C(N)=O)c1N] | 481 |
| 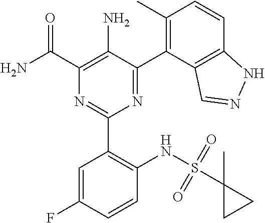 [SMILES: Cc1ccc2[nH]ncc2c1-c1nc(-c2cc(F)ccc2NS(=O)(=O)C2(C)CC2)nc(C(N)=O)c1N] | 482 |
| 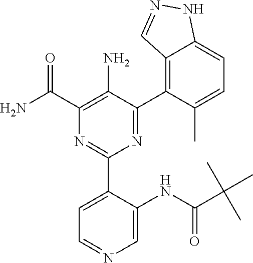 [SMILES: Cc1ccc2[nH]ncc2c1-c1nc(-c2ccncc2NC(=O)C(C)(C)C)nc(C(N)=O)c1N] | 483 |
| 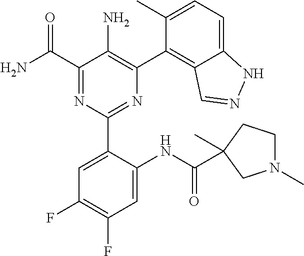 [SMILES: Cc1ccc2[nH]ncc2c1-c1nc(-c2cc(F)c(F)cc2NC(=O)C2(C)CCN(C)C2)nc(C(N)=O)c1N] | 484 |
| 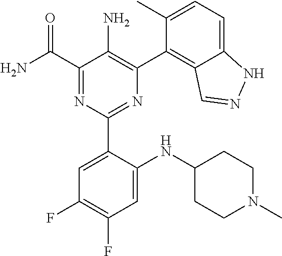 [SMILES: Cc1ccc2[nH]ncc2c1-c1nc(-c2cc(F)c(F)cc2NC2CCN(C)CC2)nc(C(N)=O)c1N] | 485 |
| 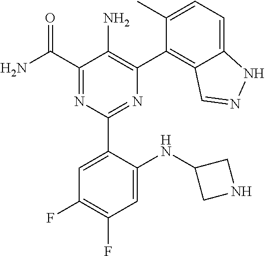 [SMILES: Cc1ccc2[nH]ncc2c1-c1nc(-c2cc(F)c(F)cc2NC2CNC2)nc(C(N)=O)c1N] | 486 |
| 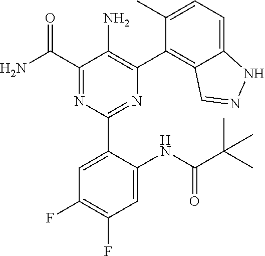 [SMILES: Cc1ccc2[nH]ncc2c1-c1nc(-c2cc(F)c(F)cc2NC(=O)C(C)(C)C)nc(C(N)=O)c1N] | 487 |
| 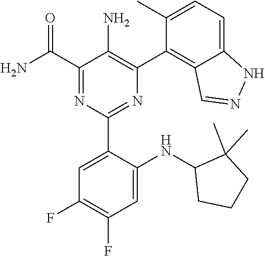 [SMILES: Cc1ccc2[nH]ncc2c1-c1nc(-c2cc(F)c(F)cc2NC2CCCC2(C)C)nc(C(N)=O)c1N] | 488 |
| 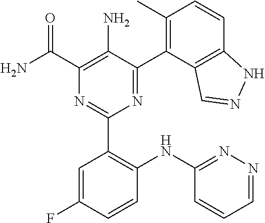 [SMILES: Cc1ccc2[nH]ncc2c1-c1nc(-c2cc(F)ccc2Nc2cccnn2)nc(C(N)=O)c1N] | 489 |
| 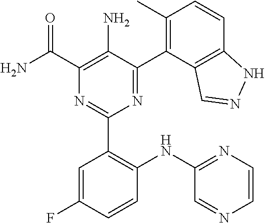 [SMILES: Cc1ccc2[nH]ncc2c1-c1nc(-c2cc(F)ccc2Nc2cnccn2)nc(C(N)=O)c1N] | 490 |
| 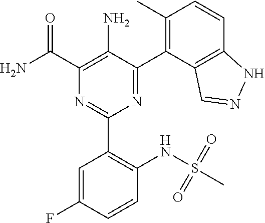 [SMILES: Cc1ccc2[nH]ncc2c1-c1nc(-c2cc(F)ccc2NS(C)(=O)=O)nc(C(N)=O)c1N] | 491 |
| 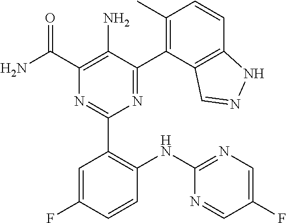 [SMILES: Cc1ccc2[nH]ncc2c1-c1nc(-c2cc(F)ccc2Nc2ncc(F)cn2)nc(C(N)=O)c1N] | 492 |
| 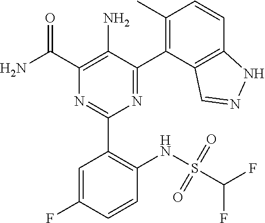 [SMILES: Cc1ccc2[nH]ncc2c1-c1nc(-c2cc(F)ccc2NS(=O)(=O)C(F)F)nc(C(N)=O)c1N] | 493 |
| 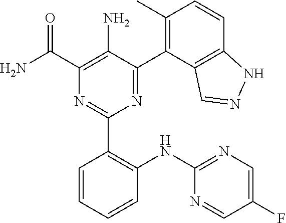 [SMILES: Cc1ccc2[nH]ncc2c1-c1nc(-c2ccccc2Nc2ncc(F)cn2)nc(C(N)=O)c1N] | 494 |
| 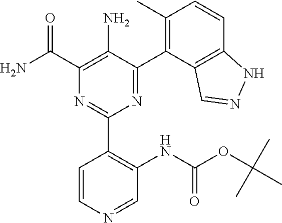 [SMILES: Cc1ccc2[nH]ncc2c1-c1nc(-c2ccncc2NC(=O)OC(C)(C)C)nc(C(N)=O)c1N] | 495 |
| 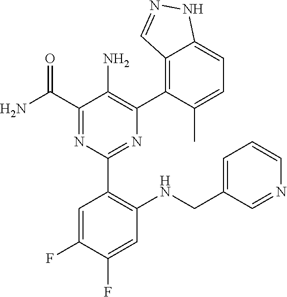 [SMILES: Cc1ccc2[nH]ncc2c1-c1nc(-c2cc(F)c(F)cc2NCc2cccnc2)nc(C(N)=O)c1N] | 496 |
| 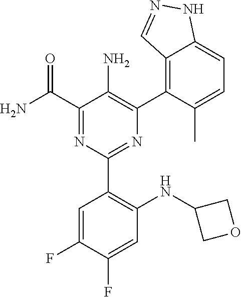 [SMILES: Cc1ccc2[nH]ncc2c1-c1nc(-c2cc(F)c(F)cc2NC2COC2)nc(C(N)=O)c1N] | 497 |
| 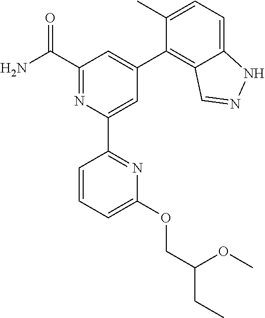 [SMILES: CCC(COc1cccc(-c2cc(-c3c(C)ccc4[nH]ncc34)cc(C(N)=O)n2)n1)OC] | 498 |
| 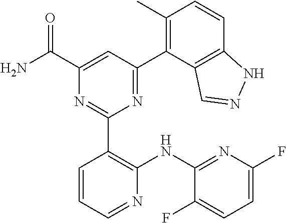 [SMILES: Cc1ccc2[nH]ncc2c1-c1cc(C(N)=O)nc(-c2cccnc2Nc2nc(F)ccc2F)n1] | 499 |
| 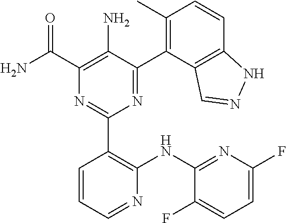 [SMILES: Cc1ccc2[nH]ncc2c1-c1nc(-c2cccnc2Nc2nc(F)ccc2F)nc(C(N)=O)c1N] | 500 |
| 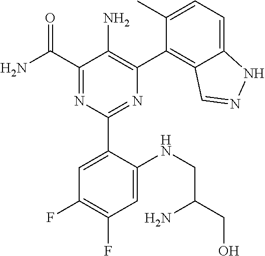 [SMILES: Cc1ccc2[nH]ncc2c1-c1nc(-c2cc(F)c(F)cc2NCC(N)CO)nc(C(N)=O)c1N] | 501 |
| 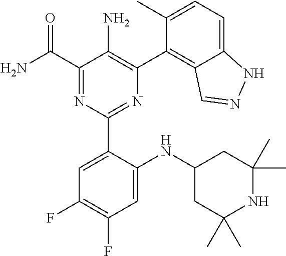 [SMILES: Cc1ccc2[nH]ncc2c1-c1nc(-c2cc(F)c(F)cc2NC2CC(C)(C)NC(C)(C)C2)nc(C(N)=O)c1N] | 502 |
| 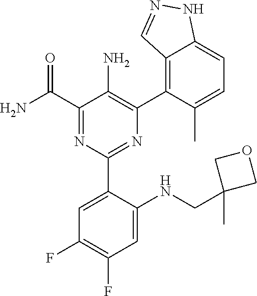 [SMILES: Cc1ccc2[nH]ncc2c1-c1nc(-c2cc(F)c(F)cc2NCC2(C)COC2)nc(C(N)=O)c1N] | 503 |
| 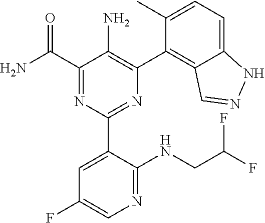 [SMILES: Cc1ccc2[nH]ncc2c1-c1nc(-c2cc(F)cnc2NCC(F)F)nc(C(N)=O)c1N] | 504 |
| 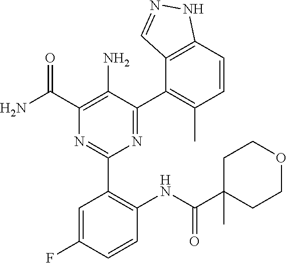 [SMILES: Cc1ccc2[nH]ncc2c1-c1nc(-c2cc(F)ccc2NC(=O)C2(C)CCOCC2)nc(C(N)=O)c1N] | 505 |
| 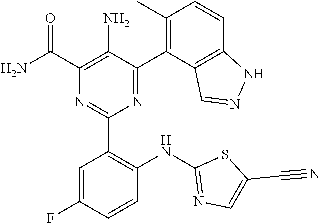 [SMILES: Cc1ccc2[nH]ncc2c1-c1nc(-c2cc(F)ccc2Nc2ncc(C#N)s2)nc(C(N)=O)c1N] | 506 |
| 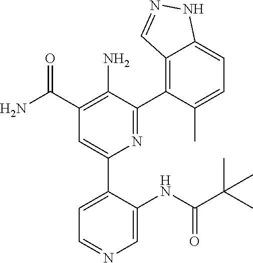 [SMILES: Cc1ccc2[nH]ncc2c1-c1nc(-c2ccncc2NC(=O)C(C)(C)C)cc(C(N)=O)c1N] | 507 |
| 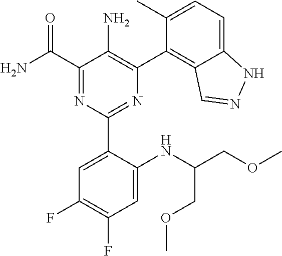 [SMILES: COCC(COC)Nc1cc(F)c(F)cc1-c1nc(C(N)=O)c(N)c(-c2c(C)ccc3[nH]ncc23)n1] | 508 |
| 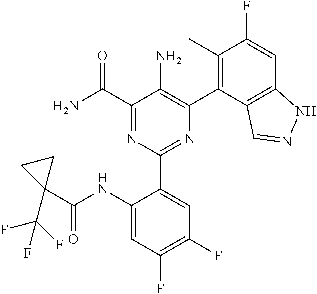 [SMILES: Cc1c(F)cc2[nH]ncc2c1-c1nc(-c2cc(F)c(F)cc2NC(=O)C2(C(F)(F)F)CC2)nc(C(N)=O)c1N] | 509 |
| 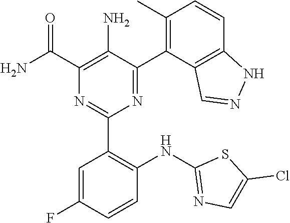 [SMILES: Cc1ccc2[nH]ncc2c1-c1nc(-c2cc(F)ccc2Nc2ncc(Cl)s2)nc(C(N)=O)c1N] | 510 |
| 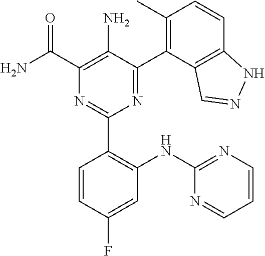 [SMILES: Cc1ccc2[nH]ncc2c1-c1nc(-c2ccc(F)cc2Nc2ncccn2)nc(C(N)=O)c1N] | 511 |
| 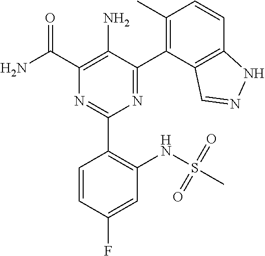 [SMILES: Cc1ccc2[nH]ncc2c1-c1nc(-c2ccc(F)cc2NS(C)(=O)=O)nc(C(N)=O)c1N] | 512 |
| 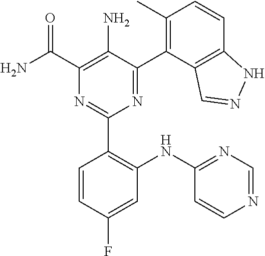 [SMILES: Cc1ccc2[nH]ncc2c1-c1nc(-c2ccc(F)cc2Nc2ccncn2)nc(C(N)=O)c1N] | 513 |
| 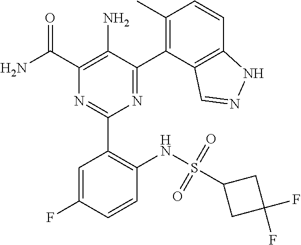 [SMILES: Cc1ccc2[nH]ncc2c1-c1nc(-c2cc(F)ccc2NS(=O)(=O)C2CC(F)(F)C2)nc(C(N)=O)c1N] | 514 |
| 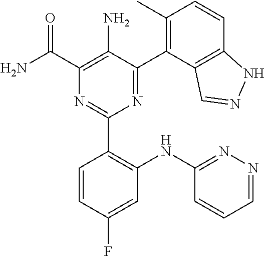 [SMILES: Cc1ccc2[nH]ncc2c1-c1nc(-c2ccc(F)cc2Nc2cccnn2)nc(C(N)=O)c1N] | 515 |
| 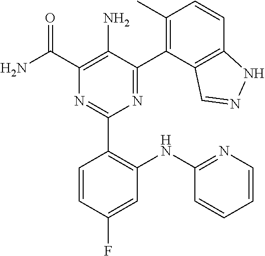 [SMILES: Cc1ccc2[nH]ncc2c1-c1nc(-c2ccc(F)cc2Nc2ccccn2)nc(C(N)=O)c1N] | 516 |
| 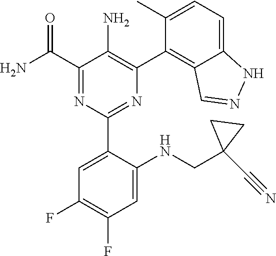 [SMILES: Cc1ccc2[nH]ncc2c1-c1nc(-c2cc(F)c(F)cc2NCC2(C#N)CC2)nc(C(N)=O)c1N] | 517 |
| 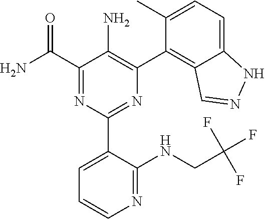 [SMILES: Cc1ccc2[nH]ncc2c1-c1nc(-c2cccnc2NCC(F)(F)F)nc(C(N)=O)c1N] | 518 |
| 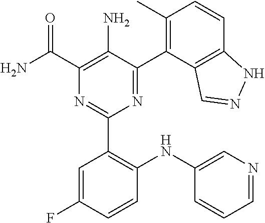 [SMILES: Cc1ccc2[nH]ncc2c1-c1nc(-c2cc(F)ccc2Nc2cccnc2)nc(C(N)=O)c1N] | 519 |
| 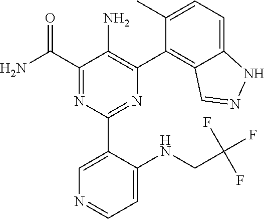 [SMILES: Cc1ccc2[nH]ncc2c1-c1nc(-c2cnccc2NCC(F)(F)F)nc(C(N)=O)c1N] | 520 |
| 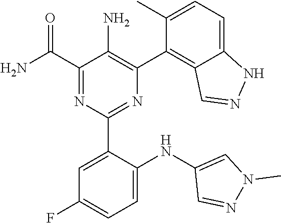 [SMILES: Cc1ccc2[nH]ncc2c1-c1nc(-c2cc(F)ccc2Nc2cnn(C)c2)nc(C(N)=O)c1N] | 521 |
| 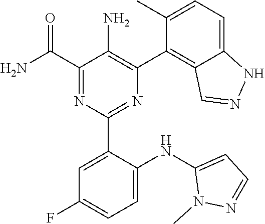 [SMILES: Cc1ccc2[nH]ncc2c1-c1nc(-c2cc(F)ccc2Nc2ccnn2C)nc(C(N)=O)c1N] | 522 |
| 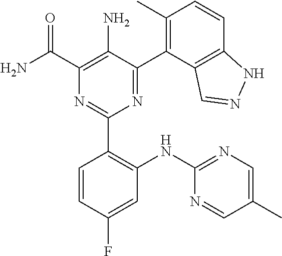 [SMILES: Cc1cnc(Nc2cc(F)ccc2-c2nc(C(N)=O)c(N)c(-c3c(C)ccc4[nH]ncc34)n2)nc1] | 523 |
| 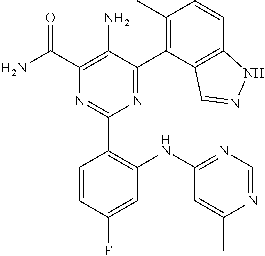 [SMILES: Cc1cc(Nc2cc(F)ccc2-c2nc(C(N)=O)c(N)c(-c3c(C)ccc4[nH]ncc34)n2)ncn1] | 524 |
| 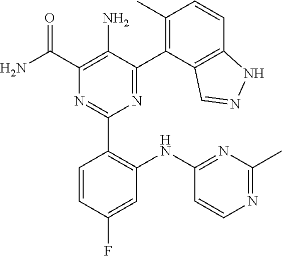 [SMILES: Cc1nccc(Nc2cc(F)ccc2-c2nc(C(N)=O)c(N)c(-c3c(C)ccc4[nH]ncc34)n2)n1] | 525 |
| 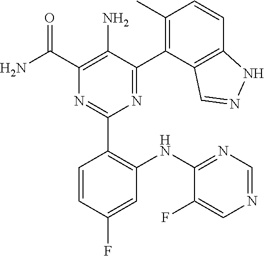 [SMILES: Cc1ccc2[nH]ncc2c1-c1nc(-c2ccc(F)cc2Nc2ncncc2F)nc(C(N)=O)c1N] | 526 |
| 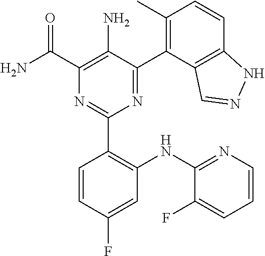 [SMILES: Cc1ccc2[nH]ncc2c1-c1nc(-c2ccc(F)cc2Nc2ncccc2F)nc(C(N)=O)c1N] | 527 |
| 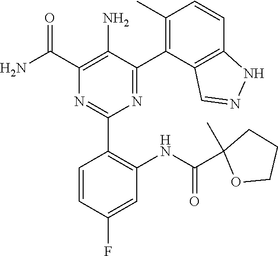 [SMILES: Cc1ccc2[nH]ncc2c1-c1nc(-c2ccc(F)cc2NC(=O)C2(C)CCCO2)nc(C(N)=O)c1N] | 528 |
| 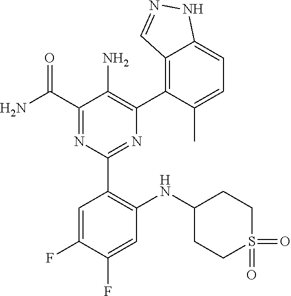 [SMILES: Cc1ccc2[nH]ncc2c1-c1nc(-c2cc(F)c(F)cc2NC2CCS(=O)(=O)CC2)nc(C(N)=O)c1N] | 529 |
| 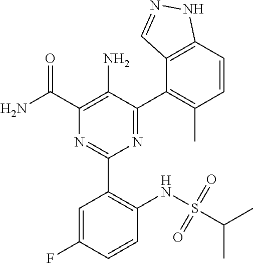 [SMILES: Cc1ccc2[nH]ncc2c1-c1nc(-c2cc(F)ccc2NS(=O)(=O)C(C)C)nc(C(N)=O)c1N] | 530 |
| 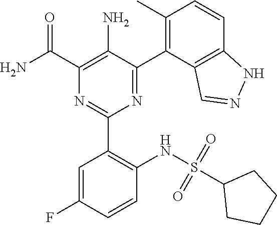 [SMILES: Cc1ccc2[nH]ncc2c1-c1nc(-c2cc(F)ccc2NS(=O)(=O)C2CCCC2)nc(C(N)=O)c1N] | 531 |
| 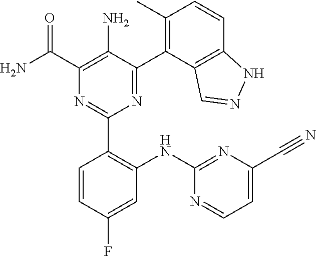 [SMILES: Cc1ccc2[nH]ncc2c1-c1nc(-c2ccc(F)cc2Nc2nccc(C#N)n2)nc(C(N)=O)c1N] | 532 |
| 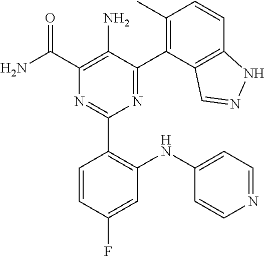 [SMILES: Cc1ccc2[nH]ncc2c1-c1nc(-c2ccc(F)cc2Nc2ccncc2)nc(C(N)=O)c1N] | 533 |
| 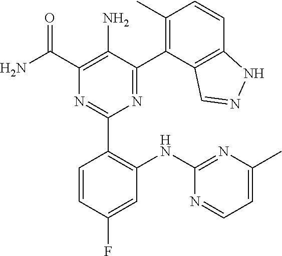 [SMILES: Cc1ccnc(Nc2cc(F)ccc2-c2nc(C(N)=O)c(N)c(-c3c(C)ccc4[nH]ncc34)n2)n1] | 534 |
| 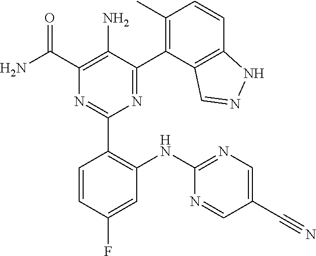 [SMILES: Cc1ccc2[nH]ncc2c1-c1nc(-c2ccc(F)cc2Nc2ncc(C#N)cn2)nc(C(N)=O)c1N] | 535 |
| 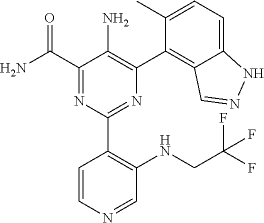 [SMILES: Cc1ccc2[nH]ncc2c1-c1nc(-c2ccncc2NCC(F)(F)F)nc(C(N)=O)c1N] | 536 |
| 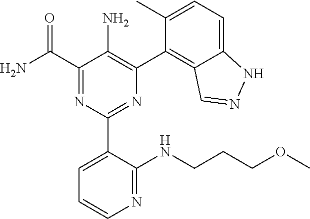 [SMILES: COCCCNc1ncccc1-c1nc(C(N)=O)c(N)c(-c2c(C)ccc3[nH]ncc23)n1] | 537 |
| 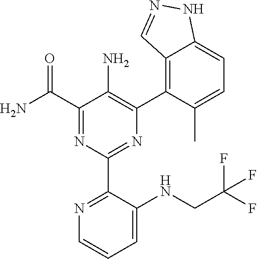 [SMILES: Cc1ccc2[nH]ncc2c1-c1nc(-c2ncccc2NCC(F)(F)F)nc(C(N)=O)c1N] | 538 |
| 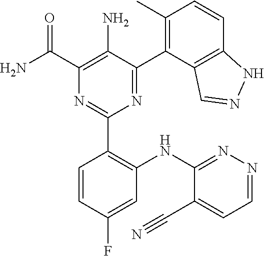 [SMILES: Cc1ccc2[nH]ncc2c1-c1nc(-c2ccc(F)cc2Nc2nnccc2C#N)nc(C(N)=O)c1N] | 539 |
| 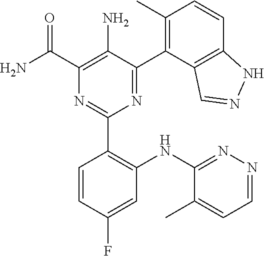 [SMILES: Cc1ccnnc1Nc1cc(F)ccc1-c1nc(C(N)=O)c(N)c(-c2c(C)ccc3[nH]ncc23)n1] | 540 |
| 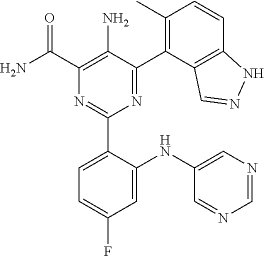 [SMILES: Cc1ccc2[nH]ncc2c1-c1nc(-c2ccc(F)cc2Nc2cncnc2)nc(C(N)=O)c1N] | 541 |
| 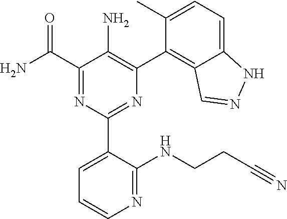 [SMILES: Cc1ccc2[nH]ncc2c1-c1nc(-c2cccnc2NCCC#N)nc(C(N)=O)c1N] | 542 |
| 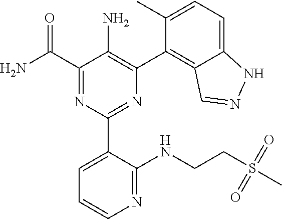 [SMILES: Cc1ccc2[nH]ncc2c1-c1nc(-c2cccnc2NCCS(C)(=O)=O)nc(C(N)=O)c1N] | 543 |
| 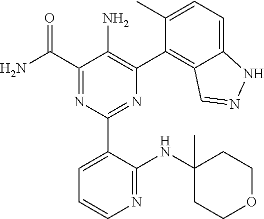 [SMILES: Cc1ccc2[nH]ncc2c1-c1nc(-c2cccnc2NC2(C)CCOCC2)nc(C(N)=O)c1N] | 544 |
| 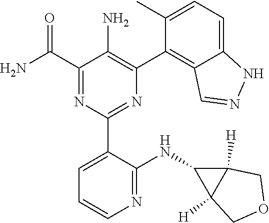 [SMILES: Cc1ccc2[nH]ncc2c1-c1nc(-c2cccnc2N[C@H]2[C@@H]3COC[C@@H]32)nc(C(N)=O)c1N] | 545 |
| 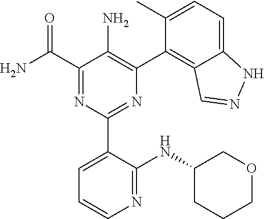 [SMILES: Cc1ccc2[nH]ncc2c1-c1nc(-c2cccnc2N[C@H]2CCCOC2)nc(C(N)=O)c1N] | 546 |
| 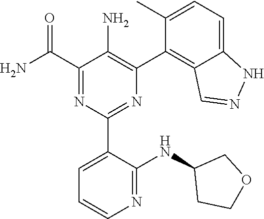 [SMILES: Cc1ccc2[nH]ncc2c1-c1nc(-c2cccnc2N[C@@H]2CCOC2)nc(C(N)=O)c1N] | 547 |
| 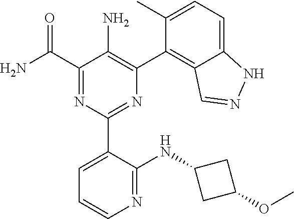 [SMILES: CO[C@H]1C[C@@H](Nc2ncccc2-c2nc(C(N)=O)c(N)c(-c3c(C)ccc4[nH]ncc34)n2)C1] | 548 |
| 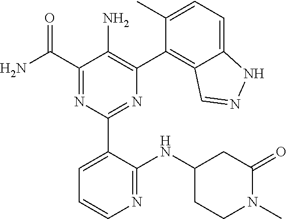 [SMILES: Cc1ccc2[nH]ncc2c1-c1nc(-c2cccnc2NC2CCN(C)C(=O)C2)nc(C(N)=O)c1N] | 549 |
| 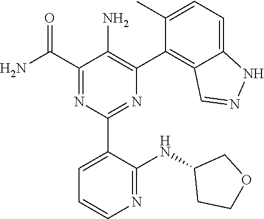 [SMILES: Cc1ccc2[nH]ncc2c1-c1nc(-c2cccnc2N[C@H]2CCOC2)nc(C(N)=O)c1N] | 550 |
| 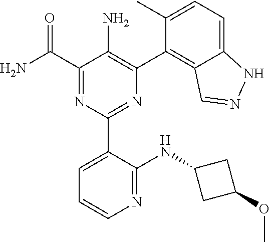 [SMILES: CO[C@H]1C[C@H](Nc2ncccc2-c2nc(C(N)=O)c(N)c(-c3c(C)ccc4[nH]ncc34)n2)C1] | 551 |
| 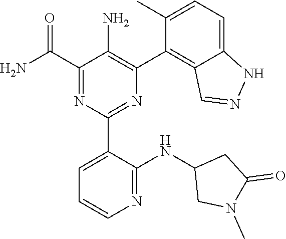 [SMILES: Cc1ccc2[nH]ncc2c1-c1nc(-c2cccnc2NC2CC(=O)N(C)C2)nc(C(N)=O)c1N] | 552 |
| 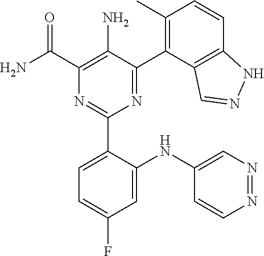 [SMILES: Cc1ccc2[nH]ncc2c1-c1nc(-c2ccc(F)cc2Nc2ccnnc2)nc(C(N)=O)c1N] | 553 |
| 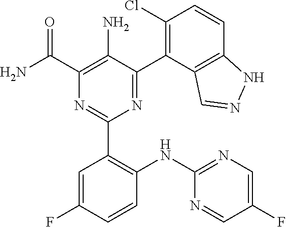 [SMILES: NC(=O)c1nc(-c2cc(F)ccc2Nc2ncc(F)cn2)nc(-c2c(Cl)ccc3[nH]ncc23)c1N] | 554 |
| 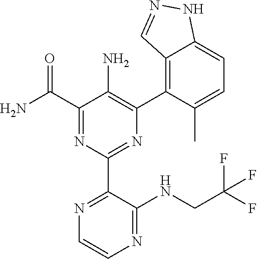 [SMILES: Cc1ccc2[nH]ncc2c1-c1nc(-c2nccnc2NCC(F)(F)F)nc(C(N)=O)c1N] | 555 |
| 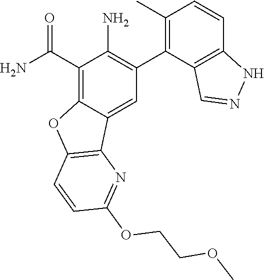 [SMILES: COCCOc1ccc2oc3c(C(N)=O)c(N)c(-c4c(C)ccc5[nH]ncc45)cc3c2n1] | 556 |
| 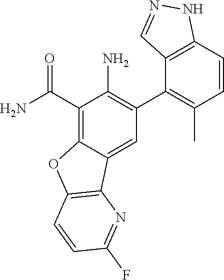 [SMILES: Cc1ccc2[nH]ncc2c1-c1cc2c(oc3ccc(F)nc32)c(C(N)=O)c1N] | 557 |
| 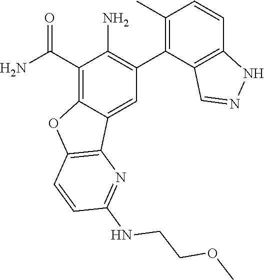 [SMILES: COCCNc1ccc2oc3c(C(N)=O)c(N)c(-c4c(C)ccc5[nH]ncc45)cc3c2n1] | 558 |
| 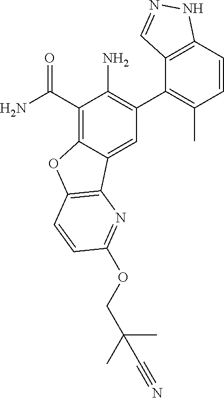 [SMILES: Cc1ccc2[nH]ncc2c1-c1cc2c(oc3ccc(OCC(C)(C)C#N)nc32)c(C(N)=O)c1N] | 559 |
| 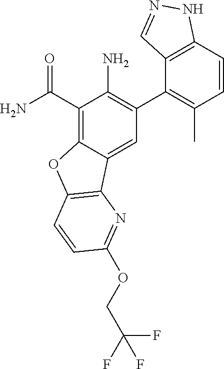 [SMILES: Cc1ccc2[nH]ncc2c1-c1cc2c(oc3ccc(OCC(F)(F)F)nc32)c(C(N)=O)c1N] | 560 |
| 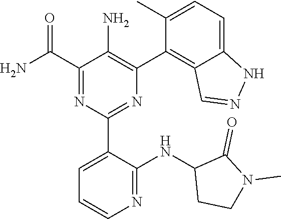 [SMILES: Cc1ccc2[nH]ncc2c1-c1nc(-c2cccnc2NC2CCN(C)C2=O)nc(C(N)=O)c1N] | 561 |
| 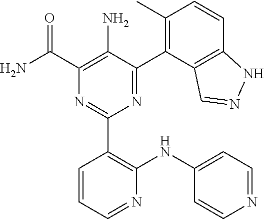 [SMILES: Cc1ccc2[nH]ncc2c1-c1nc(-c2cccnc2Nc2ccncc2)nc(C(N)=O)c1N] | 562 |
| 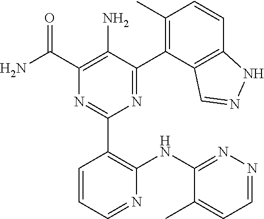 [SMILES: Cc1ccnnc1Nc1ncccc1-c1nc(C(N)=O)c(N)c(-c2c(C)ccc3[nH]ncc23)n1] | 563 |
| 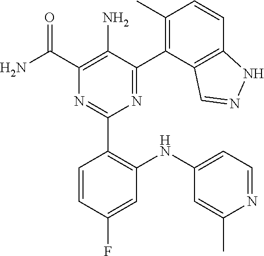 [SMILES: Cc1cc(Nc2cc(F)ccc2-c2nc(C(N)=O)c(N)c(-c3c(C)ccc4[nH]ncc34)n2)ccn1] | 564 |
| 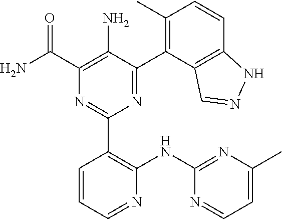 [SMILES: Cc1ccnc(Nc2ncccc2-c2nc(C(N)=O)c(N)c(-c3c(C)ccc4[nH]ncc34)n2)n1] | 565 |
| 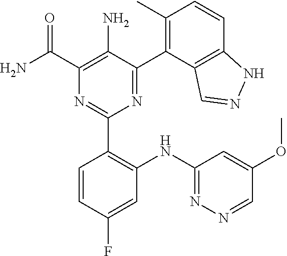 [SMILES: COc1cnnc(Nc2cc(F)ccc2-c2nc(C(N)=O)c(N)c(-c3c(C)ccc4[nH]ncc34)n2)c1] | 566 |
| 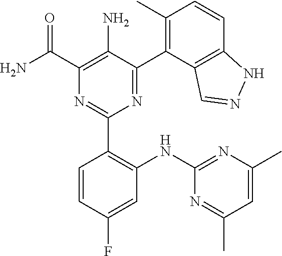 [SMILES: Cc1cc(C)nc(Nc2cc(F)ccc2-c2nc(C(N)=O)c(N)c(-c3c(C)ccc4[nH]ncc34)n2)n1] | 567 |
| 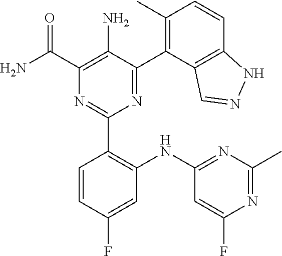 [SMILES: Cc1nc(F)cc(Nc2cc(F)ccc2-c2nc(C(N)=O)c(N)c(-c3c(C)ccc4[nH]ncc34)n2)n1] | 568 |
| 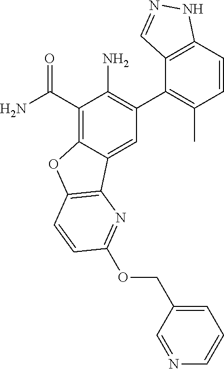 [SMILES: Cc1ccc2[nH]ncc2c1-c1cc2c(oc3ccc(OCc4cccnc4)nc32)c(C(N)=O)c1N] | 569 |
| 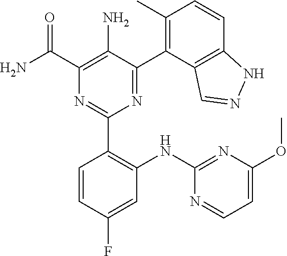 [SMILES: COc1ccnc(Nc2cc(F)ccc2-c2nc(C(N)=O)c(N)c(-c3c(C)ccc4[nH]ncc34)n2)n1] | 570 |
| 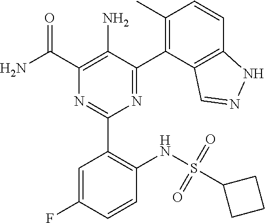 [SMILES: Cc1ccc2[nH]ncc2c1-c1nc(-c2cc(F)ccc2NS(=O)(=O)C2CCC2)nc(C(N)=O)c1N] | 571 |
| 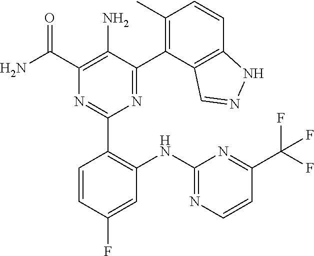 [SMILES: Cc1ccc2[nH]ncc2c1-c1nc(-c2ccc(F)cc2Nc2nccc(C(F)(F)F)n2)nc(C(N)=O)c1N] | 572 |
| 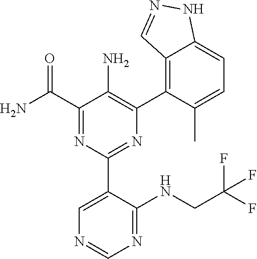 [SMILES: Cc1ccc2[nH]ncc2c1-c1nc(-c2cncnc2NCC(F)(F)F)nc(C(N)=O)c1N] | 573 |
| 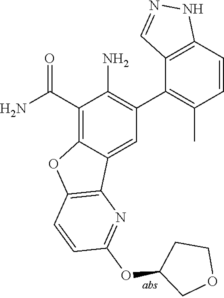 [SMILES: Cc1ccc2[nH]ncc2c1-c1cc2c(oc3ccc(O[C@H]4CCOC4)nc32)c(C(N)=O)c1N] | 574 |
| 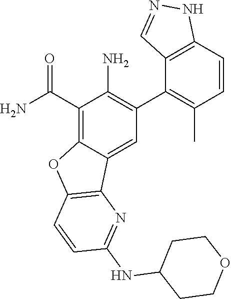 [SMILES: Cc1ccc2[nH]ncc2c1-c1cc2c(oc3ccc(NC4CCOCC4)nc32)c(C(N)=O)c1N] | 575 |
| 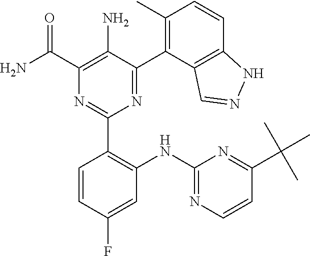 [SMILES: Cc1ccc2[nH]ncc2c1-c1nc(-c2ccc(F)cc2Nc2nccc(C(C)(C)C)n2)nc(C(N)=O)c1N] | 576 |
| 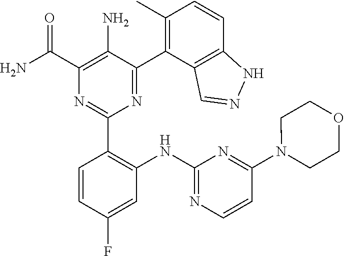 [SMILES: Cc1ccc2[nH]ncc2c1-c1nc(-c2ccc(F)cc2Nc2nccc(N3CCOCC3)n2)nc(C(N)=O)c1N] | 577 |
| 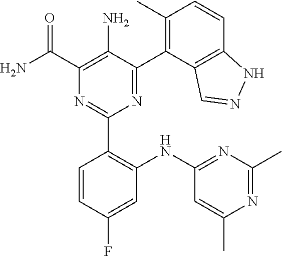 [SMILES: Cc1cc(Nc2cc(F)ccc2-c2nc(C(N)=O)c(N)c(-c3c(C)ccc4[nH]ncc34)n2)nc(C)n1] | 578 |
| 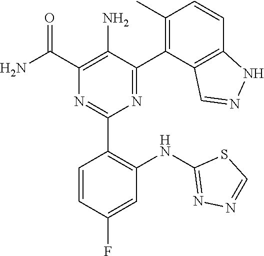 [SMILES: Cc1ccc2[nH]ncc2c1-c1nc(-c2ccc(F)cc2Nc2nncs2)nc(C(N)=O)c1N] | 579 |
| 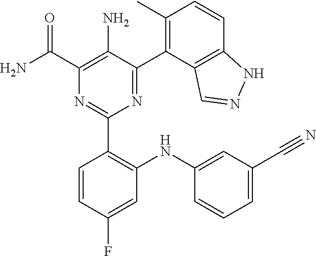 [SMILES: Cc1ccc2[nH]ncc2c1-c1nc(-c2ccc(F)cc2Nc2cccc(C#N)c2)nc(C(N)=O)c1N] | 580 |
| 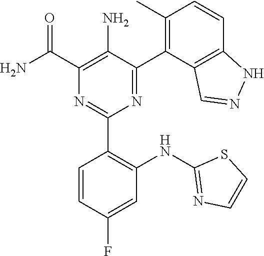 [SMILES: Cc1ccc2[nH]ncc2c1-c1nc(-c2ccc(F)cc2Nc2nccs2)nc(C(N)=O)c1N] | 581 |
| 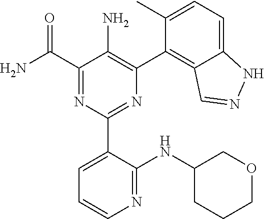 [SMILES: Cc1ccc2[nH]ncc2c1-c1nc(-c2cccnc2NC2CCCOC2)nc(C(N)=O)c1N] | 582 |
| 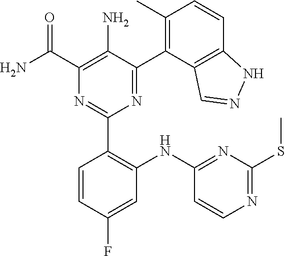 [SMILES: CSc1nccc(Nc2cc(F)ccc2-c2nc(C(N)=O)c(N)c(-c3c(C)ccc4[nH]ncc34)n2)n1] | 583 |
| 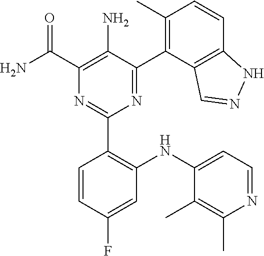 [SMILES: Cc1ccc2[nH]ncc2c1-c1nc(-c2ccc(F)cc2Nc2ccnc(C)c2C)nc(C(N)=O)c1N] | 584 |
| 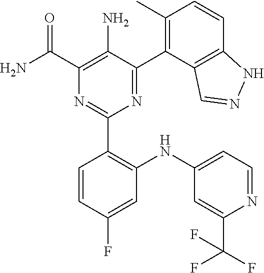 [SMILES: Cc1ccc2[nH]ncc2c1-c1nc(-c2ccc(F)cc2Nc2ccnc(C(F)(F)F)c2)nc(C(N)=O)c1N] | 585 |
| 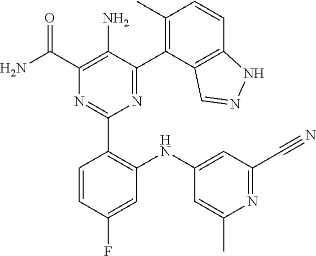 [SMILES: Cc1cc(Nc2cc(F)ccc2-c2nc(C(N)=O)c(N)c(-c3c(C)ccc4[nH]ncc34)n2)cc(C#N)n1] | 586 |
| 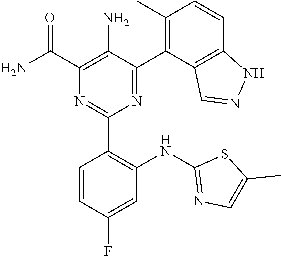 [SMILES: Cc1cnc(Nc2cc(F)ccc2-c2nc(C(N)=O)c(N)c(-c3c(C)ccc4[nH]ncc34)n2)s1] | 587 |
| 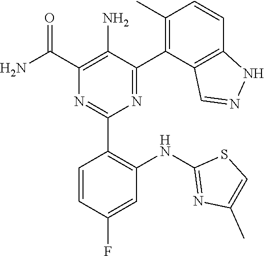 [SMILES: Cc1csc(Nc2cc(F)ccc2-c2nc(C(N)=O)c(N)c(-c3c(C)ccc4[nH]ncc34)n2)n1] | 588 |
| 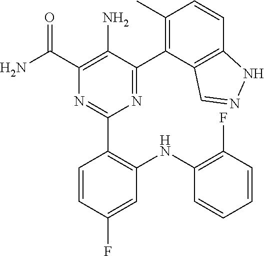 [SMILES: Cc1ccc2[nH]ncc2c1-c1nc(-c2ccc(F)cc2Nc2ccccc2F)nc(C(N)=O)c1N] | 589 |
| 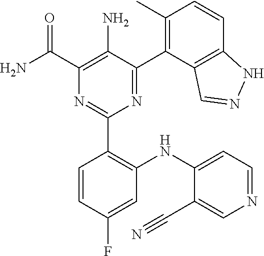 [SMILES: Cc1ccc2[nH]ncc2c1-c1nc(-c2ccc(F)cc2Nc2ccncc2C#N)nc(C(N)=O)c1N] | 590 |
| 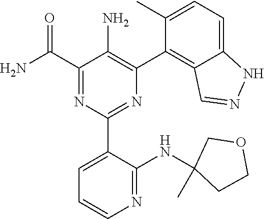 [SMILES: Cc1ccc2[nH]ncc2c1-c1nc(-c2cccnc2NC2(C)CCOC2)nc(C(N)=O)c1N] | 591 |
| 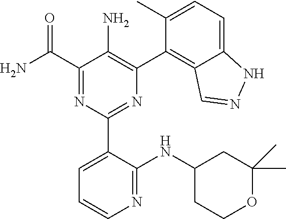 [SMILES: Cc1ccc2[nH]ncc2c1-c1nc(-c2cccnc2NC2CCOC(C)(C)C2)nc(C(N)=O)c1N] | 592 |
| 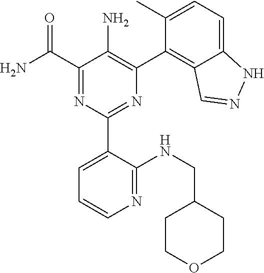 [SMILES: Cc1ccc2[nH]ncc2c1-c1nc(-c2cccnc2NCC2CCOCC2)nc(C(N)=O)c1N] | 593 |
| 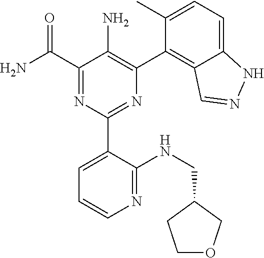 [SMILES: Cc1ccc2[nH]ncc2c1-c1nc(-c2cccnc2NC[C@H]2CCOC2)nc(C(N)=O)c1N] | 594 |
| 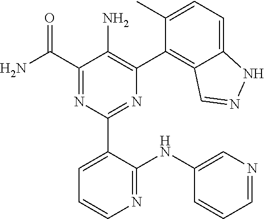 [SMILES: Cc1ccc2[nH]ncc2c1-c1nc(-c2cccnc2Nc2cccnc2)nc(C(N)=O)c1N] | 595 |
| 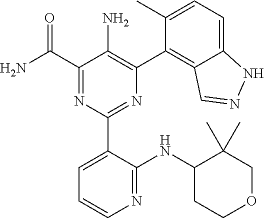 [SMILES: Cc1ccc2[nH]ncc2c1-c1nc(-c2cccnc2NC2CCOCC2(C)C)nc(C(N)=O)c1N] | 596 |
| 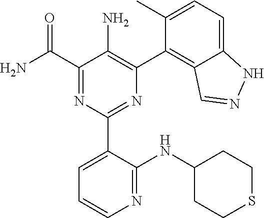 [SMILES: Cc1ccc2[nH]ncc2c1-c1nc(-c2cccnc2NC2CCSCC2)nc(C(N)=O)c1N] | 597 |
| 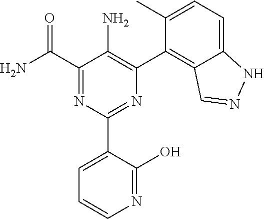 [SMILES: Cc1ccc2[nH]ncc2c1-c1nc(-c2cccnc2O)nc(C(N)=O)c1N] | 598 |
| 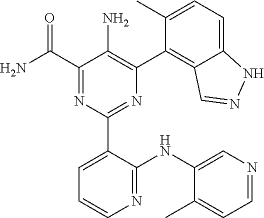 [SMILES: Cc1ccncc1Nc1ncccc1-c1nc(C(N)=O)c(N)c(-c2c(C)ccc3[nH]ncc23)n1] | 599 |
| 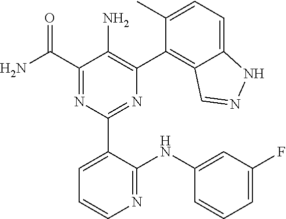 [SMILES: Cc1ccc2[nH]ncc2c1-c1nc(-c2cccnc2Nc2cccc(F)c2)nc(C(N)=O)c1N] | 600 |
| 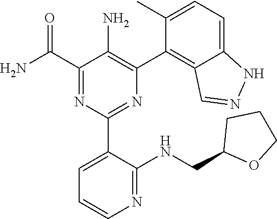 [SMILES: Cc1ccc2[nH]ncc2c1-c1nc(-c2cccnc2NC[C@H]2CCCO2)nc(C(N)=O)c1N] | 601 |
| 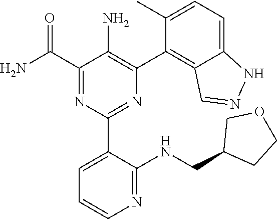 [SMILES: Cc1ccc2[nH]ncc2c1-c1nc(-c2cccnc2NC[C@@H]2CCOC2)nc(C(N)=O)c1N] | 602 |
| 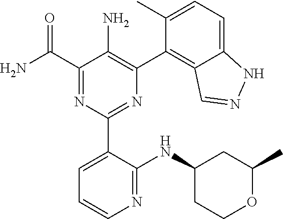 [SMILES: Cc1ccc2[nH]ncc2c1-c1nc(-c2cccnc2N[C@@H]2CCO[C@H](C)C2)nc(C(N)=O)c1N] | 603 |
| 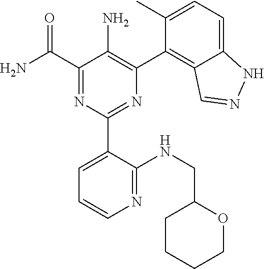 [SMILES: Cc1ccc2[nH]ncc2c1-c1nc(-c2cccnc2NCC2CCCCO2)nc(C(N)=O)c1N] | 604 |
| 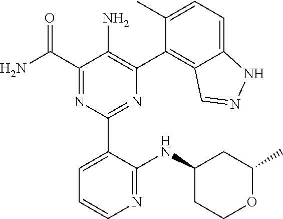 [SMILES: Cc1ccc2[nH]ncc2c1-c1nc(-c2cccnc2N[C@@H]2CCO[C@@H](C)C2)nc(C(N)=O)c1N] | 605 |
| 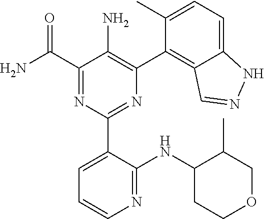 [SMILES: Cc1ccc2[nH]ncc2c1-c1nc(-c2cccnc2NC2CCOCC2C)nc(C(N)=O)c1N] | 606 |
| 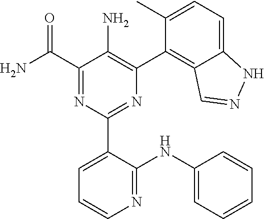 [SMILES: Cc1ccc2[nH]ncc2c1-c1nc(-c2cccnc2Nc2ccccc2)nc(C(N)=O)c1N] | 607 |
| 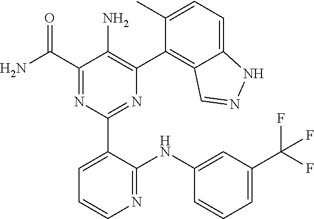 [SMILES: Cc1ccc2[nH]ncc2c1-c1nc(-c2cccnc2Nc2cccc(C(F)(F)F)c2)nc(C(N)=O)c1N] | 608 |
| 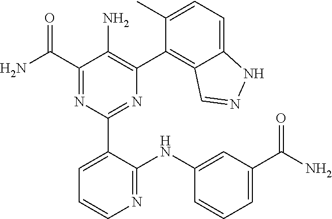 [SMILES: Cc1ccc2[nH]ncc2c1-c1nc(-c2cccnc2Nc2cccc(C(N)=O)c2)nc(C(N)=O)c1N] | 609 |
| 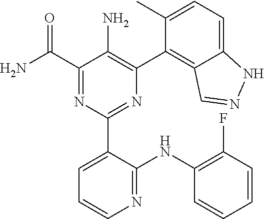 [SMILES: Cc1ccc2[nH]ncc2c1-c1nc(-c2cccnc2Nc2ccccc2F)nc(C(N)=O)c1N] | 610 |
| 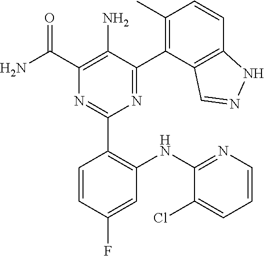 [SMILES: Cc1ccc2[nH]ncc2c1-c1nc(-c2ccc(F)cc2Nc2ncccc2Cl)nc(C(N)=O)c1N] | 611 |
| 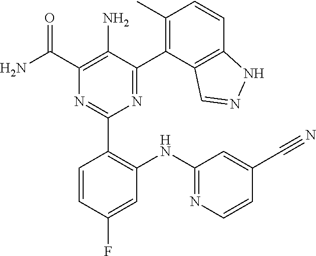 [SMILES: Cc1ccc2[nH]ncc2c1-c1nc(-c2ccc(F)cc2Nc2cc(C#N)ccn2)nc(C(N)=O)c1N] | 612 |
| 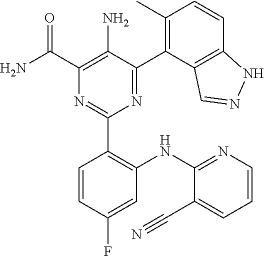 [SMILES: Cc1ccc2[nH]ncc2c1-c1nc(-c2ccc(F)cc2Nc2ncccc2C#N)nc(C(N)=O)c1N] | 613 |
| 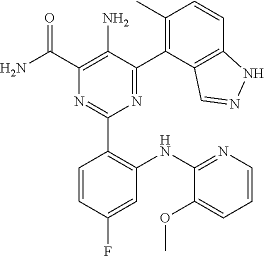 [SMILES: COc1cccnc1Nc1cc(F)ccc1-c1nc(C(N)=O)c(N)c(-c2c(C)ccc3[nH]ncc23)n1] | 614 |
| 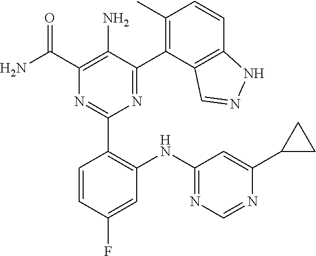 [SMILES: Cc1ccc2[nH]ncc2c1-c1nc(-c2ccc(F)cc2Nc2cc(C3CC3)ncn2)nc(C(N)=O)c1N] | 615 |
| 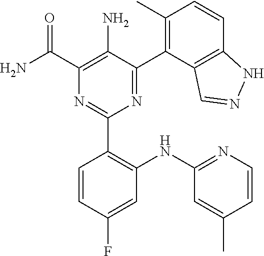 [SMILES: Cc1ccnc(Nc2cc(F)ccc2-c2nc(C(N)=O)c(N)c(-c3c(C)ccc4[nH]ncc34)n2)c1] | 616 |
| 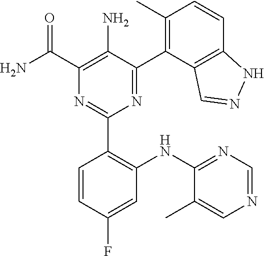 [SMILES: Cc1cncnc1Nc1cc(F)ccc1-c1nc(C(N)=O)c(N)c(-c2c(C)ccc3[nH]ncc23)n1] | 617 |
| 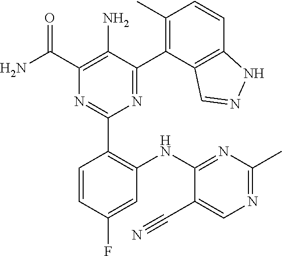 [SMILES: Cc1ncc(C#N)c(Nc2cc(F)ccc2-c2nc(C(N)=O)c(N)c(-c3c(C)ccc4[nH]ncc34)n2)n1] | 618 |
| 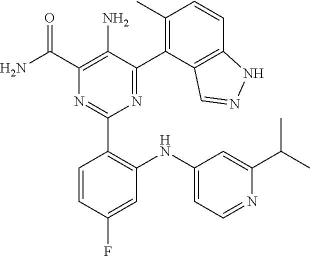 [SMILES: Cc1ccc2[nH]ncc2c1-c1nc(-c2ccc(F)cc2Nc2ccnc(C(C)C)c2)nc(C(N)=O)c1N] | 619 |
| 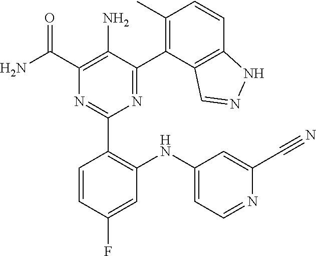 [SMILES: Cc1ccc2[nH]ncc2c1-c1nc(-c2ccc(F)cc2Nc2ccnc(C#N)c2)nc(C(N)=O)c1N] | 620 |
| 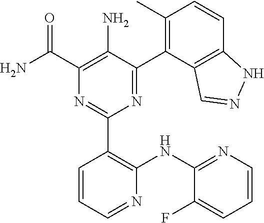 [SMILES: Cc1ccc2[nH]ncc2c1-c1nc(-c2cccnc2Nc2ncccc2F)nc(C(N)=O)c1N] | 621 |
| 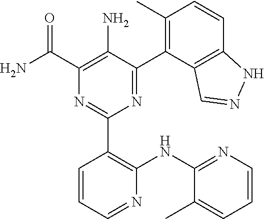 [SMILES: Cc1cccnc1Nc1ncccc1-c1nc(C(N)=O)c(N)c(-c2c(C)ccc3[nH]ncc23)n1] | 622 |
| 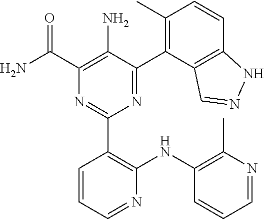 [SMILES: Cc1ccc2[nH]ncc2c1-c1nc(-c2cccnc2Nc2cccnc2C)nc(C(N)=O)c1N] | 623 |
| 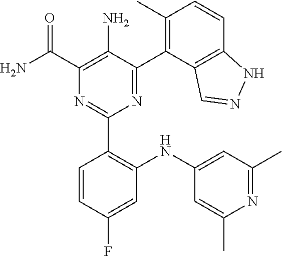 [SMILES: Cc1cc(Nc2cc(F)ccc2-c2nc(C(N)=O)c(N)c(-c3c(C)ccc4[nH]ncc34)n2)cc(C)n1] | 624 |
| 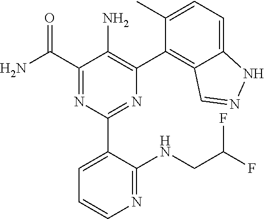 [SMILES: Cc1ccc2[nH]ncc2c1-c1nc(-c2cccnc2NCC(F)F)nc(C(N)=O)c1N] | 625 |
| 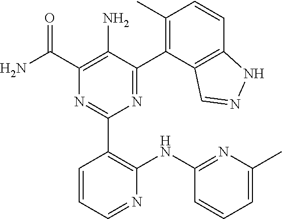 [SMILES: Cc1cccc(Nc2ncccc2-c2nc(C(N)=O)c(N)c(-c3c(C)ccc4[nH]ncc34)n2)n1] | 626 |
| 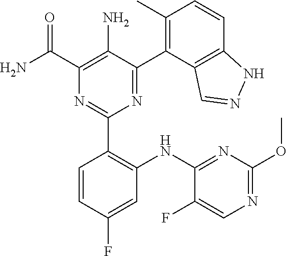 [SMILES: COc1ncc(F)c(Nc2cc(F)ccc2-c2nc(C(N)=O)c(N)c(-c3c(C)ccc4[nH]ncc34)n2)n1] | 627 |
| 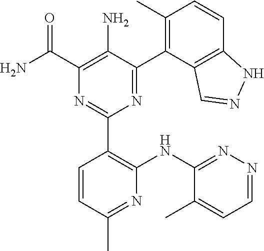 [SMILES: Cc1ccc(-c2nc(C(N)=O)c(N)c(-c3c(C)ccc4[nH]ncc34)n2)c(Nc2nnccc2C)n1] | 628 |
| 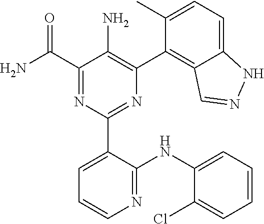 [SMILES: Cc1ccc2[nH]ncc2c1-c1nc(-c2cccnc2Nc2ccccc2Cl)nc(C(N)=O)c1N] | 629 |
| 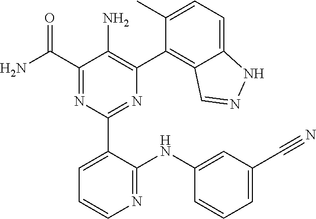 [SMILES: Cc1ccc2[nH]ncc2c1-c1nc(-c2cccnc2Nc2cccc(C#N)c2)nc(C(N)=O)c1N] | 630 |
| 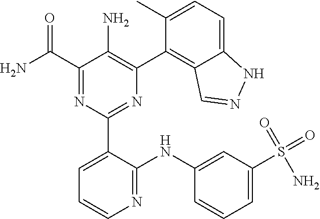 [SMILES: Cc1ccc2[nH]ncc2c1-c1nc(-c2cccnc2Nc2cccc(S(N)(=O)=O)c2)nc(C(N)=O)c1N] | 631 |
| 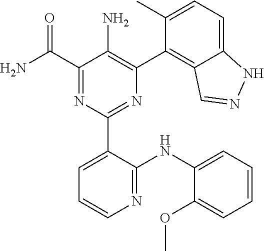 [SMILES: COc1ccccc1Nc1ncccc1-c1nc(C(N)=O)c(N)c(-c2c(C)ccc3[nH]ncc23)n1] | 632 |
| 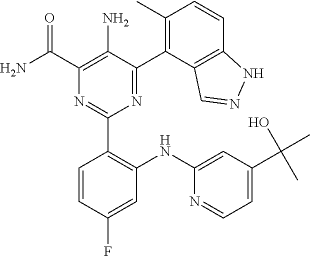 [SMILES: Cc1ccc2[nH]ncc2c1-c1nc(-c2ccc(F)cc2Nc2cc(C(C)(C)O)ccn2)nc(C(N)=O)c1N] | 633 |
| 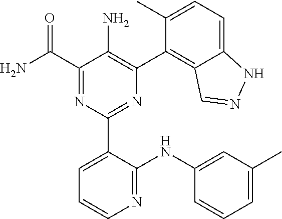 [SMILES: Cc1cccc(Nc2ncccc2-c2nc(C(N)=O)c(N)c(-c3c(C)ccc4[nH]ncc34)n2)c1] | 634 |
| 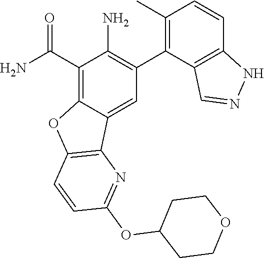 [SMILES: Cc1ccc2[nH]ncc2c1-c1cc2c(oc3ccc(OC4CCOCC4)nc32)c(C(N)=O)c1N] | 635 |
| 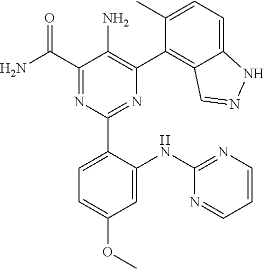 [SMILES: COc1ccc(-c2nc(C(N)=O)c(N)c(-c3c(C)ccc4[nH]ncc34)n2)c(Nc2ncccn2)c1] | 636 |
| 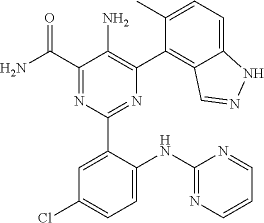 [SMILES: Cc1ccc2[nH]ncc2c1-c1nc(-c2cc(Cl)ccc2Nc2ncccn2)nc(C(N)=O)c1N] | 637 |
| 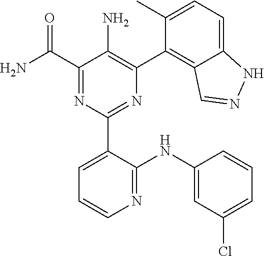 [SMILES: Cc1ccc2[nH]ncc2c1-c1nc(-c2cccnc2Nc2cccc(Cl)c2)nc(C(N)=O)c1N] | 638 |
| 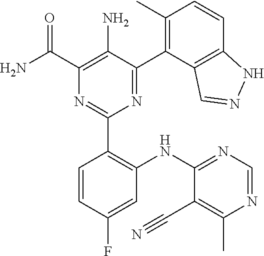 [SMILES: Cc1ccc2[nH]ncc2c1-c1nc(-c2ccc(F)cc2Nc2ncnc(C)c2C#N)nc(C(N)=O)c1N] | 639 |
| 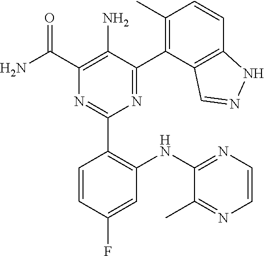 [SMILES: Cc1ccc2[nH]ncc2c1-c1nc(-c2ccc(F)cc2Nc2nccnc2C)nc(C(N)=O)c1N] | 640 |
| 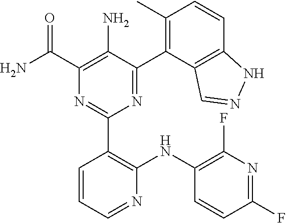 [SMILES: Cc1ccc2[nH]ncc2c1-c1nc(-c2cccnc2Nc2ccc(F)nc2F)nc(C(N)=O)c1N] | 641 |
| 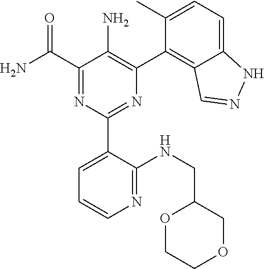 [SMILES: Cc1ccc2[nH]ncc2c1-c1nc(-c2cccnc2NCC2COCCO2)nc(C(N)=O)c1N] | 642 |
| 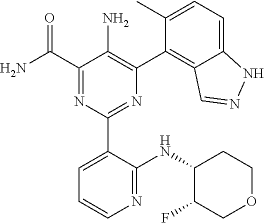 [SMILES: Cc1ccc2[nH]ncc2c1-c1nc(-c2cccnc2N[C@@H]2CCOC[C@@H]2F)nc(C(N)=O)c1N] | 643 |
| 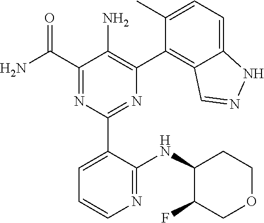 [SMILES: Cc1ccc2[nH]ncc2c1-c1nc(-c2cccnc2N[C@H]2CCOC[C@H]2F)nc(C(N)=O)c1N] | 644 |
| 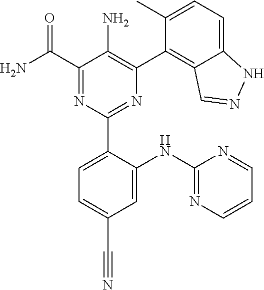 [SMILES: Cc1ccc2[nH]ncc2c1-c1nc(-c2ccc(C#N)cc2Nc2ncccn2)nc(C(N)=O)c1N] | 645 |
| 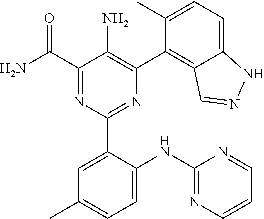 [SMILES: Cc1ccc(Nc2ncccn2)c(-c2nc(C(N)=O)c(N)c(-c3c(C)ccc4[nH]ncc34)n2)c1] | 646 |
| 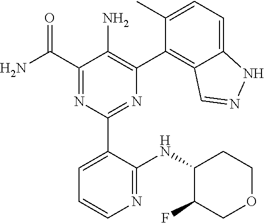 [SMILES: Cc1ccc2[nH]ncc2c1-c1nc(-c2cccnc2N[C@@H]2CCOC[C@H]2F)nc(C(N)=O)c1N] | 647 |
| 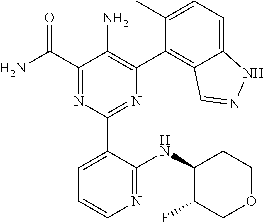 [SMILES: Cc1ccc2[nH]ncc2c1-c1nc(-c2cccnc2N[C@H]2CCOC[C@@H]2F)nc(C(N)=O)c1N] | 648 |
| 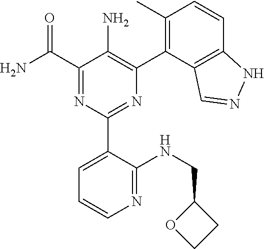 [SMILES: Cc1ccc2[nH]ncc2c1-c1nc(-c2cccnc2NC[C@H]2CCO2)nc(C(N)=O)c1N] | 649 |
| 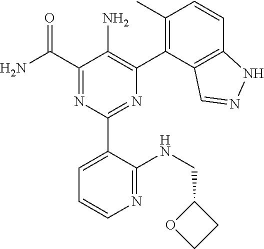 [SMILES: Cc1ccc2[nH]ncc2c1-c1nc(-c2cccnc2NC[C@@H]2CCO2)nc(C(N)=O)c1N] | 650 |
| 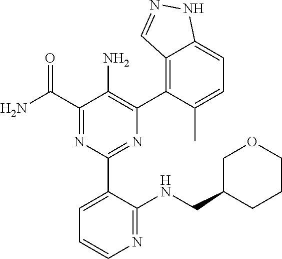 [SMILES: Cc1ccc2[nH]ncc2c1-c1nc(-c2cccnc2NC[C@@H]2CCCOC2)nc(C(N)=O)c1N] | 651 |
| 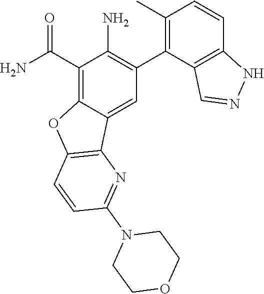 [SMILES: Cc1ccc2[nH]ncc2c1-c1cc2c(oc3ccc(N4CCOCC4)nc32)c(C(N)=O)c1N] | 652 |
| 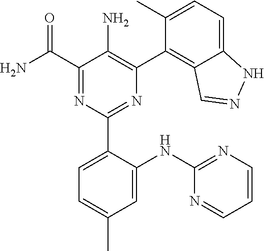 [SMILES: Cc1ccc(-c2nc(C(N)=O)c(N)c(-c3c(C)ccc4[nH]ncc34)n2)c(Nc2ncccn2)c1] | 653 |
| 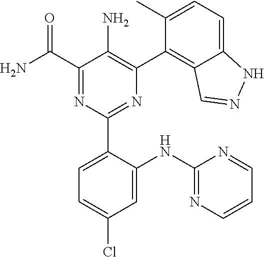 [SMILES: Cc1ccc2[nH]ncc2c1-c1nc(-c2ccc(Cl)cc2Nc2ncccn2)nc(C(N)=O)c1N] | 654 |
| 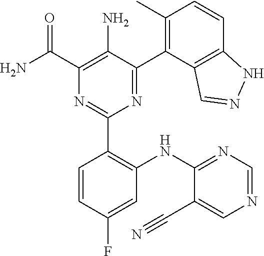 [SMILES: Cc1ccc2[nH]ncc2c1-c1nc(-c2ccc(F)cc2Nc2ncncc2C#N)nc(C(N)=O)c1N] | 655 |
| 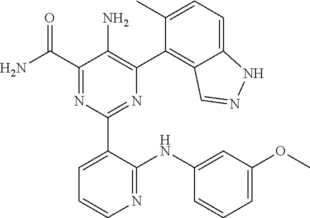 [SMILES: COc1cccc(Nc2ncccc2-c2nc(C(N)=O)c(N)c(-c3c(C)ccc4[nH]ncc34)n2)c1] | 656 |
| 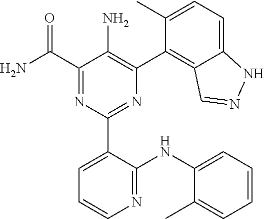 [SMILES: Cc1ccccc1Nc1ncccc1-c1nc(C(N)=O)c(N)c(-c2c(C)ccc3[nH]ncc23)n1] | 657 |
| 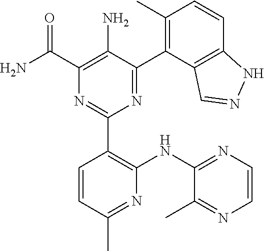 [SMILES: Cc1ccc(-c2nc(C(N)=O)c(N)c(-c3c(C)ccc4[nH]ncc34)n2)c(Nc2nccnc2C)n1] | 658 |
| 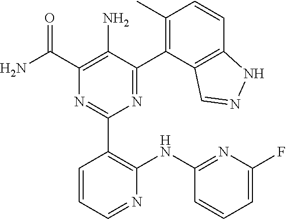 [SMILES: Cc1ccc2[nH]ncc2c1-c1nc(-c2cccnc2Nc2cccc(F)n2)nc(C(N)=O)c1N] | 659 |
| 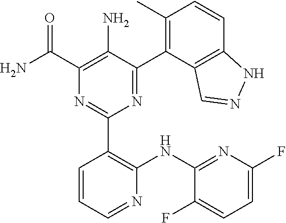 [SMILES: Cc1ccc2[nH]ncc2c1-c1nc(-c2cccnc2Nc2nc(F)ccc2F)nc(C(N)=O)c1N] | 660 |
| 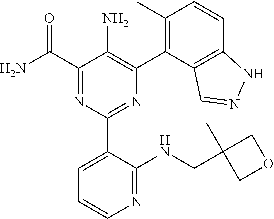 [SMILES: Cc1ccc2[nH]ncc2c1-c1nc(-c2cccnc2NCC2(C)COC2)nc(C(N)=O)c1N] | 661 |
| 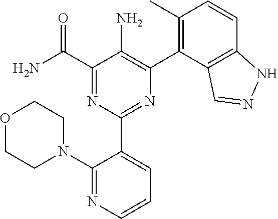 [SMILES: Cc1ccc2[nH]ncc2c1-c1nc(-c2cccnc2N2CCOCC2)nc(C(N)=O)c1N] | 662 |
| 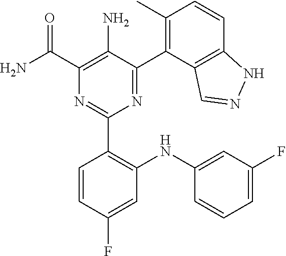 [SMILES: Cc1ccc2[nH]ncc2c1-c1nc(-c2ccc(F)cc2Nc2cccc(F)c2)nc(C(N)=O)c1N] | 663 |
| 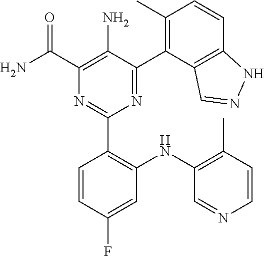 [SMILES: Cc1ccncc1Nc1cc(F)ccc1-c1nc(C(N)=O)c(N)c(-c2c(C)ccc3[nH]ncc23)n1] | 664 |
| 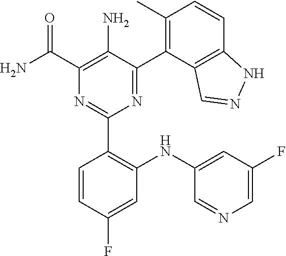 [SMILES: Cc1ccc2[nH]ncc2c1-c1nc(-c2ccc(F)cc2Nc2cncc(F)c2)nc(C(N)=O)c1N] | 665 |
| 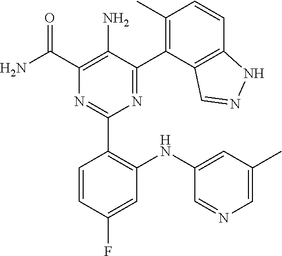 [SMILES: Cc1cncc(Nc2cc(F)ccc2-c2nc(C(N)=O)c(N)c(-c3c(C)ccc4[nH]ncc34)n2)c1] | 666 |
| 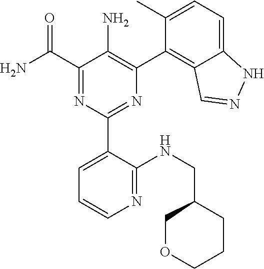 [SMILES: Cc1ccc2[nH]ncc2c1-c1nc(-c2cccnc2NC[C@H]2CCCOC2)nc(C(N)=O)c1N] | 667 |
| 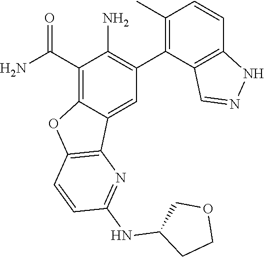 [SMILES: Cc1ccc2[nH]ncc2c1-c1cc2c(oc3ccc(N[C@H]4CCOC4)nc32)c(C(N)=O)c1N] | 668 |
| 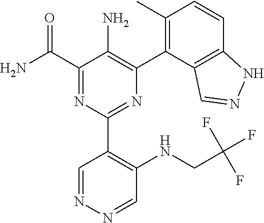 [SMILES: Cc1ccc2[nH]ncc2c1-c1nc(-c2cnncc2NCC(F)(F)F)nc(C(N)=O)c1N] | 669 |
| 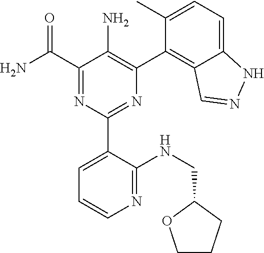 [SMILES: Cc1ccc2[nH]ncc2c1-c1nc(-c2cccnc2NC[C@@H]2CCCO2)nc(C(N)=O)c1N] | 670 |
| 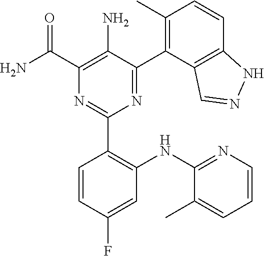 [SMILES: Cc1cccnc1Nc1cc(F)ccc1-c1nc(C(N)=O)c(N)c(-c2c(C)ccc3[nH]ncc23)n1] | 671 |
| 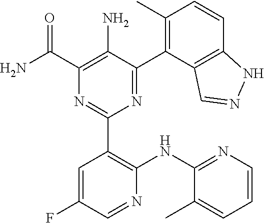 [SMILES: Cc1cccnc1Nc1ncc(F)cc1-c1nc(C(N)=O)c(N)c(-c2c(C)ccc3[nH]ncc23)n1] | 672 |
| 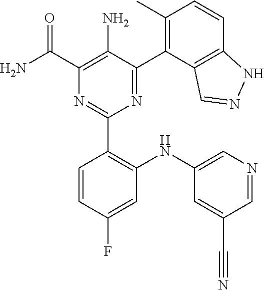 [SMILES: Cc1ccc2[nH]ncc2c1-c1nc(-c2ccc(F)cc2Nc2cncc(C#N)c2)nc(C(N)=O)c1N] | 673 |
| 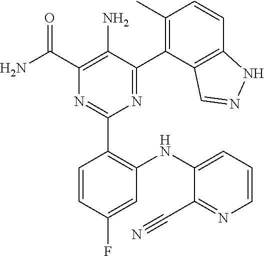 [SMILES: Cc1ccc2[nH]ncc2c1-c1nc(-c2ccc(F)cc2Nc2cccnc2C#N)nc(C(N)=O)c1N] | 674 |
| 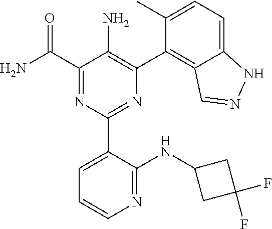 [SMILES: Cc1ccc2[nH]ncc2c1-c1nc(-c2cccnc2NC2CC(F)(F)C2)nc(C(N)=O)c1N] | 675 |
| 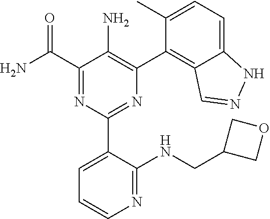 [SMILES: Cc1ccc2[nH]ncc2c1-c1nc(-c2cccnc2NCC2COC2)nc(C(N)=O)c1N] | 676 |
| 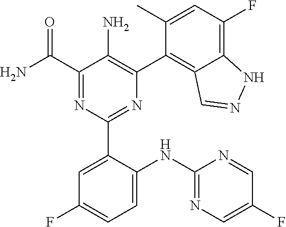 [SMILES: Cc1cc(F)c2[nH]ncc2c1-c1nc(-c2cc(F)ccc2Nc2ncc(F)cn2)nc(C(N)=O)c1N] | 677 |
| 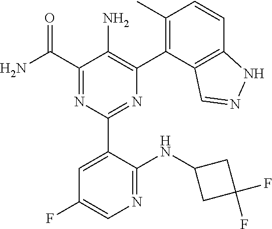 [SMILES: Cc1ccc2[nH]ncc2c1-c1nc(-c2cc(F)cnc2NC2CC(F)(F)C2)nc(C(N)=O)c1N] | 678 |
| 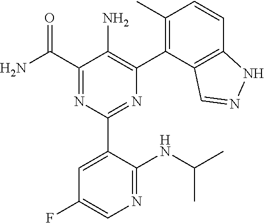 [SMILES: Cc1ccc2[nH]ncc2c1-c1nc(-c2cc(F)cnc2NC(C)C)nc(C(N)=O)c1N] | 679 |
| 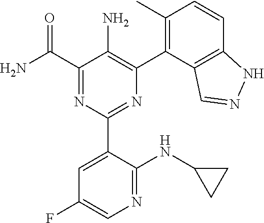 [SMILES: Cc1ccc2[nH]ncc2c1-c1nc(-c2cc(F)cnc2NC2CC2)nc(C(N)=O)c1N] | 680 |
| 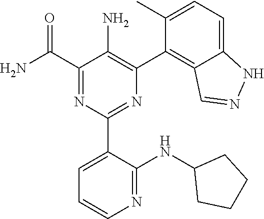 [SMILES: Cc1ccc2[nH]ncc2c1-c1nc(-c2cccnc2NC2CCCC2)nc(C(N)=O)c1N] | 681 |
| 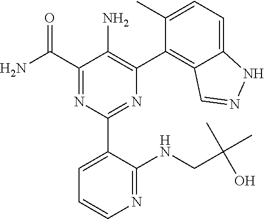 [SMILES: Cc1ccc2[nH]ncc2c1-c1nc(-c2cccnc2NCC(C)(C)O)nc(C(N)=O)c1N] | 682 |
| 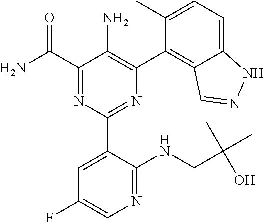 [SMILES: Cc1ccc2[nH]ncc2c1-c1nc(-c2cc(F)cnc2NCC(C)(C)O)nc(C(N)=O)c1N] | 683 |
| 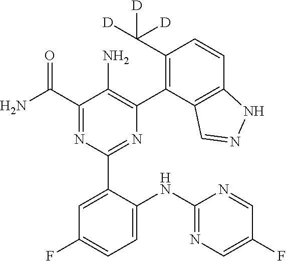 [SMILES: [2H]C([2H])([2H])c1ccc2[nH]ncc2c1-c1nc(-c2cc(F)ccc2Nc2ncc(F)cn2)nc(C(N)=O)c1N] | 684 |
| 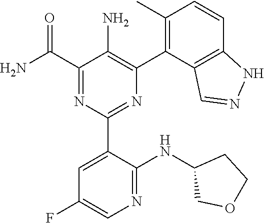 [SMILES: Cc1ccc2[nH]ncc2c1-c1nc(-c2cc(F)cnc2N[C@@H]2CCOC2)nc(C(N)=O)c1N] | 685 |
| 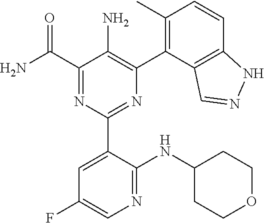 [SMILES: Cc1ccc2[nH]ncc2c1-c1nc(-c2cc(F)cnc2NC2CCOCC2)nc(C(N)=O)c1N] | 686 |
| 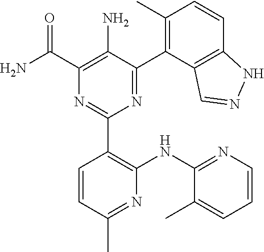 [SMILES: Cc1ccc(-c2nc(C(N)=O)c(N)c(-c3c(C)ccc4[nH]ncc34)n2)c(Nc2ncccc2C)n1] | 687 |
| 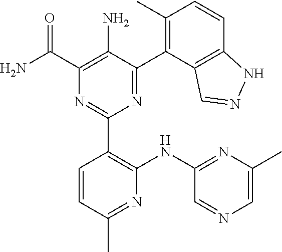 [SMILES: Cc1cncc(Nc2nc(C)ccc2-c2nc(C(N)=O)c(N)c(-c3c(C)ccc4[nH]ncc34)n2)n1] | 688 |
| 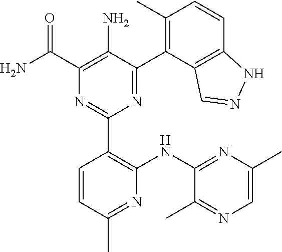 [SMILES: Cc1ccc(-c2nc(C(N)=O)c(N)c(-c3c(C)ccc4[nH]ncc34)n2)c(Nc2nc(C)cnc2C)n1] | 689 |
| 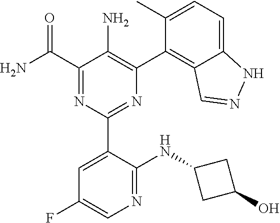 [SMILES: Cc1ccc2[nH]ncc2c1-c1nc(-c2cc(F)cnc2N[C@H]2C[C@H](O)C2)nc(C(N)=O)c1N] | 690 |
| 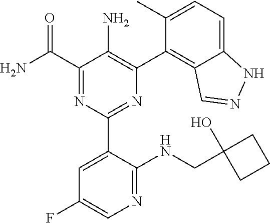 [SMILES: Cc1ccc2[nH]ncc2c1-c1nc(-c2cc(F)cnc2NCC2(O)CCC2)nc(C(N)=O)c1N] | 691 |
| 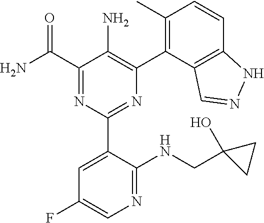 [SMILES: Cc1ccc2[nH]ncc2c1-c1nc(-c2cc(F)cnc2NCC2(O)CC2)nc(C(N)=O)c1N] | 692 |
| 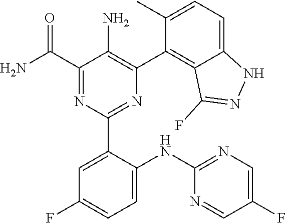 [SMILES: Cc1ccc2[nH]nc(F)c2c1-c1nc(-c2cc(F)ccc2Nc2ncc(F)cn2)nc(C(N)=O)c1N] | 693 |
| 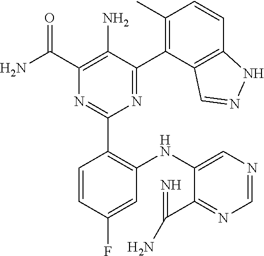 [SMILES: Cc1ccc2[nH]ncc2c1-c1nc(-c2ccc(F)cc2Nc2cncnc2C(=N)N)nc(C(N)=O)c1N] | 694 |
| 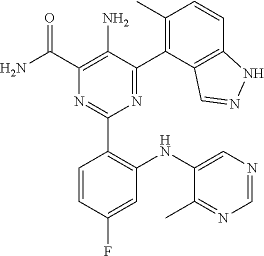 [SMILES: Cc1ccc2[nH]ncc2c1-c1nc(-c2ccc(F)cc2Nc2cncnc2C)nc(C(N)=O)c1N] | 695 |
| 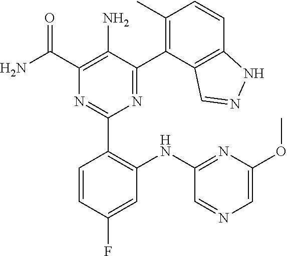 [SMILES: COc1cncc(Nc2cc(F)ccc2-c2nc(C(N)=O)c(N)c(-c3c(C)ccc4[nH]ncc34)n2)n1] | 696 |
| 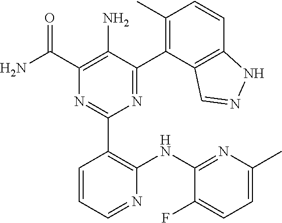 [SMILES: Cc1ccc(F)c(Nc2ncccc2-c2nc(C(N)=O)c(N)c(-c3c(C)ccc4[nH]ncc34)n2)n1] | 697 |
| 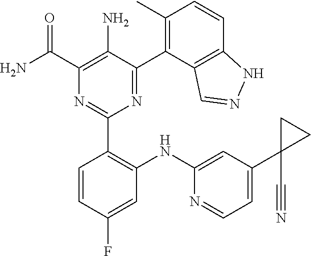 [SMILES: Cc1ccc2[nH]ncc2c1-c1nc(-c2ccc(F)cc2Nc2cc(C3(C#N)CC3)ccn2)nc(C(N)=O)c1N] | 698 |
| 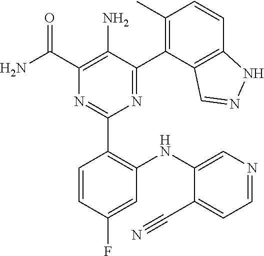 [SMILES: Cc1ccc2[nH]ncc2c1-c1nc(-c2ccc(F)cc2Nc2cnccc2C#N)nc(C(N)=O)c1N] | 699 |
| 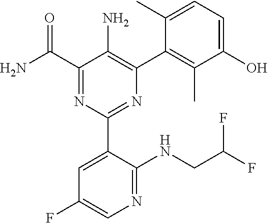 [SMILES: Cc1ccc(O)c(C)c1-c1nc(-c2cc(F)cnc2NCC(F)F)nc(C(N)=O)c1N] | 700 |
| 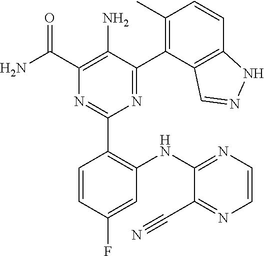 [SMILES: Cc1ccc2[nH]ncc2c1-c1nc(-c2ccc(F)cc2Nc2nccnc2C#N)nc(C(N)=O)c1N] | 701 |
| 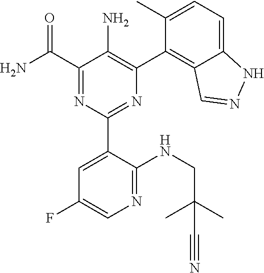 [SMILES: Cc1ccc2[nH]ncc2c1-c1nc(-c2cc(F)cnc2NCC(C)(C)C#N)nc(C(N)=O)c1N] | 702 |
| 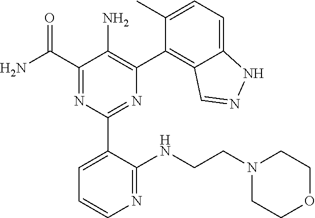 [SMILES: Cc1ccc2[nH]ncc2c1-c1nc(-c2cccnc2NCCN2CCOCC2)nc(C(N)=O)c1N] | 703 |
| 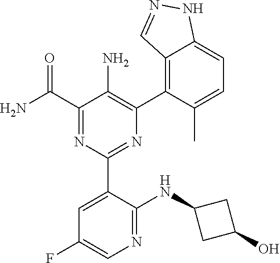 [SMILES: Cc1ccc2[nH]ncc2c1-c1nc(-c2cc(F)cnc2N[C@H]2C[C@@H](O)C2)nc(C(N)=O)c1N] | 704 |
| 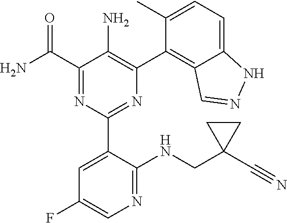 [SMILES: Cc1ccc2[nH]ncc2c1-c1nc(-c2cc(F)cnc2NCC2(C#N)CC2)nc(C(N)=O)c1N] | 705 |
| 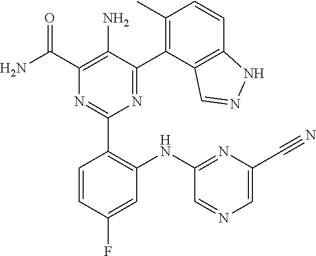 [SMILES: Cc1ccc2[nH]ncc2c1-c1nc(-c2ccc(F)cc2Nc2cncc(C#N)n2)nc(C(N)=O)c1N] | 706 |
| 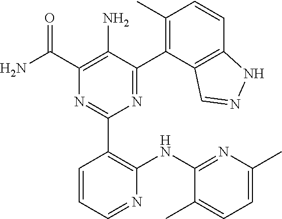 [SMILES: Cc1ccc(C)c(Nc2ncccc2-c2nc(C(N)=O)c(N)c(-c3c(C)ccc4[nH]ncc34)n2)n1] | 707 |
| 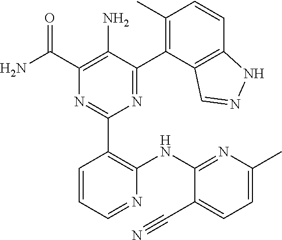 [SMILES: Cc1ccc(C#N)c(Nc2ncccc2-c2nc(C(N)=O)c(N)c(-c3c(C)ccc4[nH]ncc34)n2)n1] | 708 |
| 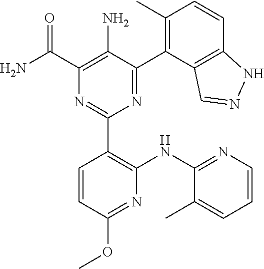 [SMILES: COc1ccc(-c2nc(C(N)=O)c(N)c(-c3c(C)ccc4[nH]ncc34)n2)c(Nc2ncccc2C)n1] | 709 |
| 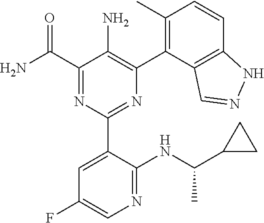 [SMILES: Cc1ccc2[nH]ncc2c1-c1nc(-c2cc(F)cnc2N[C@@H](C)C2CC2)nc(C(N)=O)c1N] | 710 |
| 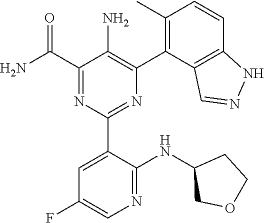 [SMILES: Cc1ccc2[nH]ncc2c1-c1nc(-c2cc(F)cnc2N[C@H]2CCOC2)nc(C(N)=O)c1N] | 711 |
| 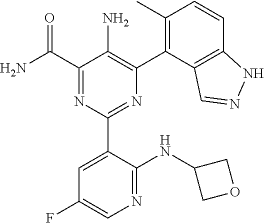 [SMILES: Cc1ccc2[nH]ncc2c1-c1nc(-c2cc(F)cnc2NC2COC2)nc(C(N)=O)c1N] | 712 |
| 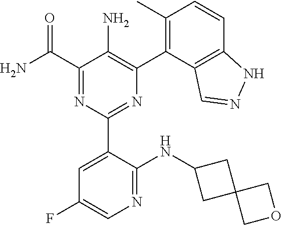 [SMILES: Cc1ccc2[nH]ncc2c1-c1nc(-c2cc(F)cnc2NC2CC3(COC3)C2)nc(C(N)=O)c1N] | 713 |
| 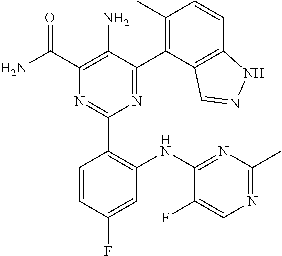 [SMILES: Cc1ncc(F)c(Nc2cc(F)ccc2-c2nc(C(N)=O)c(N)c(-c3c(C)ccc4[nH]ncc34)n2)n1] | 714 |
| 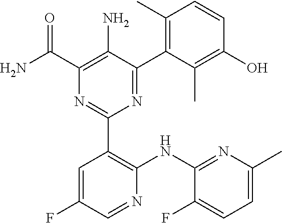 [SMILES: Cc1ccc(F)c(Nc2ncc(F)cc2-c2nc(C(N)=O)c(N)c(-c3c(C)ccc(O)c3C)n2)n1] | 715 |
| 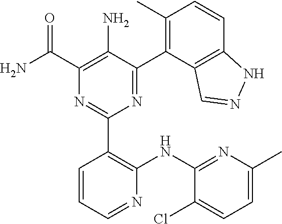 [SMILES: Cc1ccc(Cl)c(Nc2ncccc2-c2nc(C(N)=O)c(N)c(-c3c(C)ccc4[nH]ncc34)n2)n1] | 716 |
| 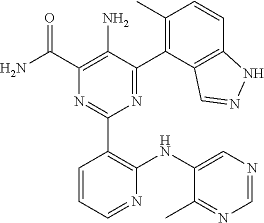 [SMILES: Cc1ccc2[nH]ncc2c1-c1nc(-c2cccnc2Nc2cncnc2C)nc(C(N)=O)c1N] | 717 |
| 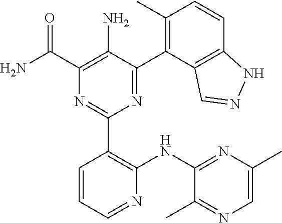 [SMILES: Cc1cnc(C)c(Nc2ncccc2-c2nc(C(N)=O)c(N)c(-c3c(C)ccc4[nH]ncc34)n2)n1] | 718 |
| 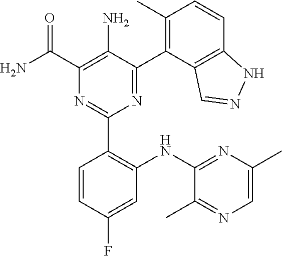 [SMILES: Cc1cnc(C)c(Nc2cc(F)ccc2-c2nc(C(N)=O)c(N)c(-c3c(C)ccc4[nH]ncc34)n2)n1] | 719 |
| 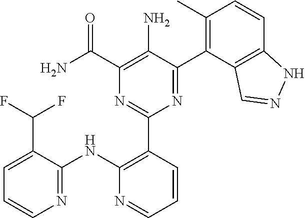 [SMILES: Cc1ccc2[nH]ncc2c1-c1nc(-c2cccnc2Nc2ncccc2C(F)F)nc(C(N)=O)c1N] | 720 |
| 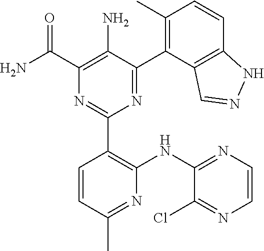 [SMILES: Cc1ccc(-c2nc(C(N)=O)c(N)c(-c3c(C)ccc4[nH]ncc34)n2)c(Nc2nccnc2Cl)n1] | 721 |
| 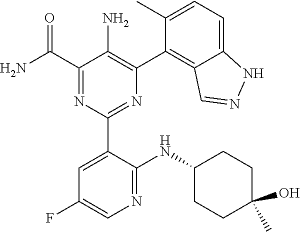 [SMILES: Cc1ccc2[nH]ncc2c1-c1nc(-c2cc(F)cnc2N[C@H]2CC[C@](C)(O)CC2)nc(C(N)=O)c1N] | 722 |
| 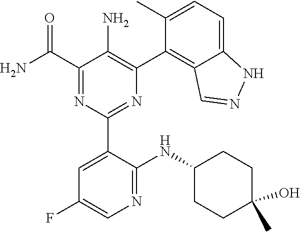 [SMILES: Cc1ccc2[nH]ncc2c1-c1nc(-c2cc(F)cnc2N[C@H]2CC[C@@](C)(O)CC2)nc(C(N)=O)c1N] | 723 |
| 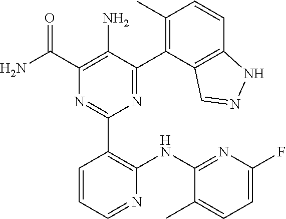 [SMILES: Cc1ccc(F)nc1Nc1ncccc1-c1nc(C(N)=O)c(N)c(-c2c(C)ccc3[nH]ncc23)n1] | 724 |
| 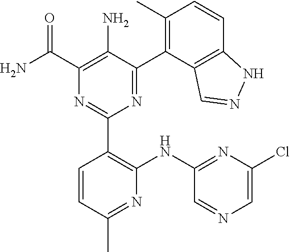 [SMILES: Cc1ccc(-c2nc(C(N)=O)c(N)c(-c3c(C)ccc4[nH]ncc34)n2)c(Nc2cncc(Cl)n2)n1] | 725 |
| 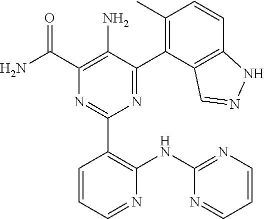 [SMILES: Cc1ccc2[nH]ncc2c1-c1nc(-c2cccnc2Nc2ncccn2)nc(C(N)=O)c1N] | 726 |
| 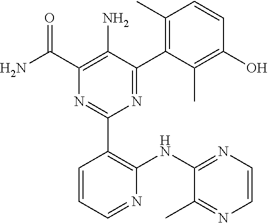 [SMILES: Cc1ccc(O)c(C)c1-c1nc(-c2cccnc2Nc2nccnc2C)nc(C(N)=O)c1N] | 727 |
| 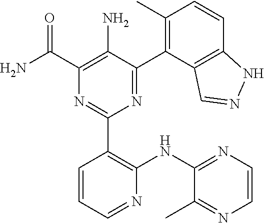 [SMILES: Cc1ccc2[nH]ncc2c1-c1nc(-c2cccnc2Nc2nccnc2C)nc(C(N)=O)c1N] | 728 |
| 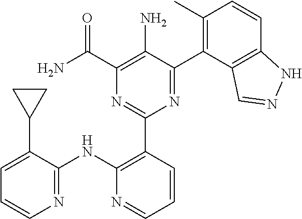 [SMILES: Cc1ccc2[nH]ncc2c1-c1nc(-c2cccnc2Nc2ncccc2C2CC2)nc(C(N)=O)c1N] | 729 |
| 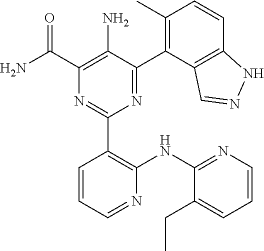 [SMILES: CCc1cccnc1Nc1ncccc1-c1nc(C(N)=O)c(N)c(-c2c(C)ccc3[nH]ncc23)n1] | 730 |
| 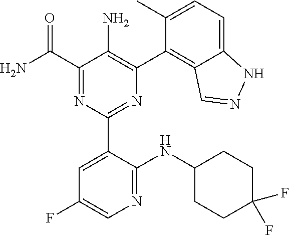 [SMILES: Cc1ccc2[nH]ncc2c1-c1nc(-c2cc(F)cnc2NC2CCC(F)(F)CC2)nc(C(N)=O)c1N] | 731 |
| 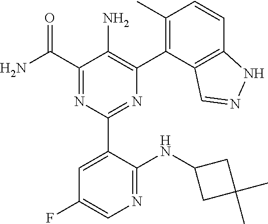 [SMILES: Cc1ccc2[nH]ncc2c1-c1nc(-c2cc(F)cnc2NC2CC(C)(C)C2)nc(C(N)=O)c1N] | 732 |
| 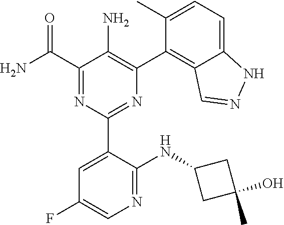 [SMILES: Cc1ccc2[nH]ncc2c1-c1nc(-c2cc(F)cnc2N[C@H]2C[C@@](C)(O)C2)nc(C(N)=O)c1N] | 733 |
| 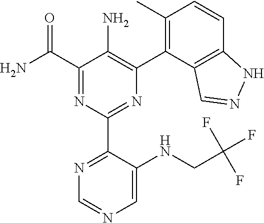 [SMILES: Cc1ccc2[nH]ncc2c1-c1nc(-c2ncncc2NCC(F)(F)F)nc(C(N)=O)c1N] | 734 |
| 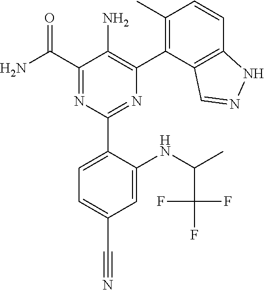 [SMILES: Cc1ccc2[nH]ncc2c1-c1nc(-c2ccc(C#N)cc2NC(C)C(F)(F)F)nc(C(N)=O)c1N] | 735 |
| 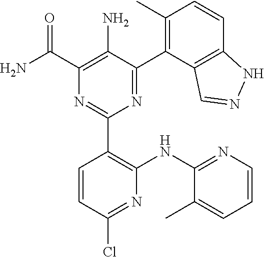 [SMILES: Cc1cccnc1Nc1nc(Cl)ccc1-c1nc(C(N)=O)c(N)c(-c2c(C)ccc3[nH]ncc23)n1] | 736 |
| 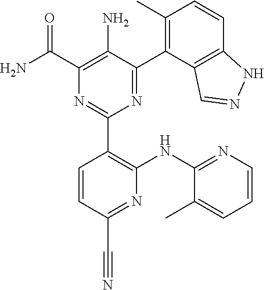 [SMILES: Cc1cccnc1Nc1nc(C#N)ccc1-c1nc(C(N)=O)c(N)c(-c2c(C)ccc3[nH]ncc23)n1] | 737 |
| 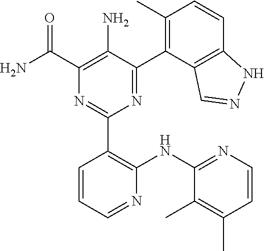 [SMILES: Cc1ccnc(Nc2ncccc2-c2nc(C(N)=O)c(N)c(-c3c(C)ccc4[nH]ncc34)n2)c1C] | 738 |
| 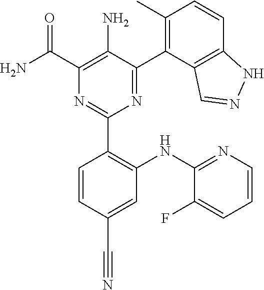 [SMILES: Cc1ccc2[nH]ncc2c1-c1nc(-c2ccc(C#N)cc2Nc2ncccc2F)nc(C(N)=O)c1N] | 739 |
| 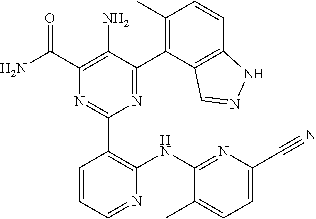 [SMILES: Cc1ccc(C#N)nc1Nc1ncccc1-c1nc(C(N)=O)c(N)c(-c2c(C)ccc3[nH]ncc23)n1] | 740 |
| 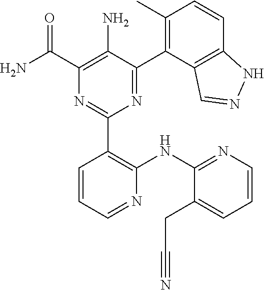 [SMILES: Cc1ccc2[nH]ncc2c1-c1nc(-c2cccnc2Nc2ncccc2CC#N)nc(C(N)=O)c1N] | 741 |
| 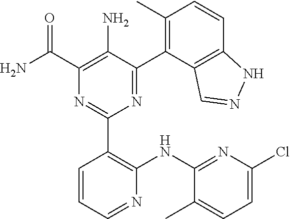 [SMILES: Cc1ccc(Cl)nc1Nc1ncccc1-c1nc(C(N)=O)c(N)c(-c2c(C)ccc3[nH]ncc23)n1] | 742 |
| 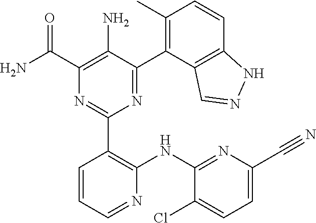 [SMILES: Cc1ccc2[nH]ncc2c1-c1nc(-c2cccnc2Nc2nc(C#N)ccc2Cl)nc(C(N)=O)c1N] | 743 |
| 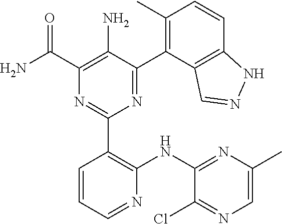 [SMILES: Cc1cnc(Cl)c(Nc2ncccc2-c2nc(C(N)=O)c(N)c(-c3c(C)ccc4[nH]ncc34)n2)n1] | 744 |
| 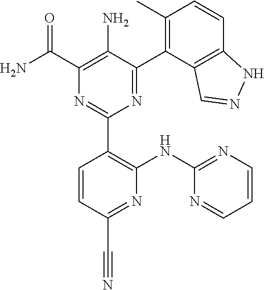 [SMILES: Cc1ccc2[nH]ncc2c1-c1nc(-c2ccc(C#N)nc2Nc2ncccn2)nc(C(N)=O)c1N] | 745 |
| 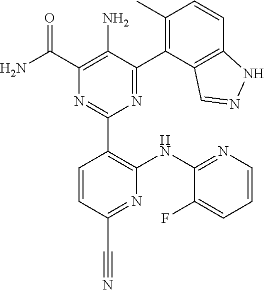 [SMILES: Cc1ccc2[nH]ncc2c1-c1nc(-c2ccc(C#N)nc2Nc2ncccc2F)nc(C(N)=O)c1N] | 746 |
| 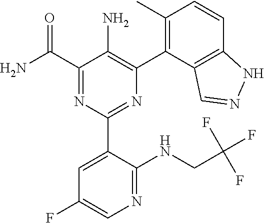 [SMILES: Cc1ccc2[nH]ncc2c1-c1nc(-c2cc(F)cnc2NCC(F)(F)F)nc(C(N)=O)c1N] | 747 |
| 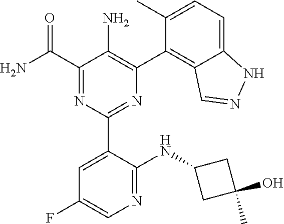 [SMILES: Cc1ccc2[nH]ncc2c1-c1nc(-c2cc(F)cnc2N[C@H]2C[C@](C)(O)C2)nc(C(N)=O)c1N] | 748 |
| 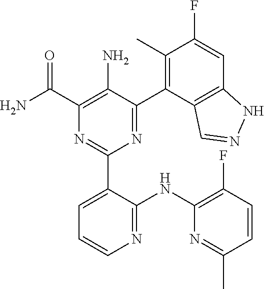 [SMILES: Cc1ccc(F)c(Nc2ncccc2-c2nc(C(N)=O)c(N)c(-c3c(C)c(F)cc4[nH]ncc34)n2)n1] | 749 |
| 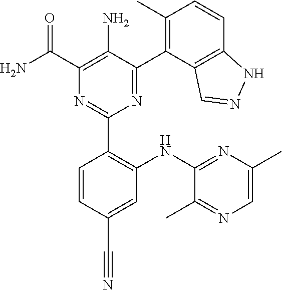 [SMILES: Cc1cnc(C)c(Nc2cc(C#N)ccc2-c2nc(C(N)=O)c(N)c(-c3c(C)ccc4[nH]ncc34)n2)n1] | 750 |
| 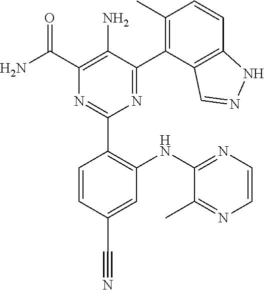 [SMILES: Cc1ccc2[nH]ncc2c1-c1nc(-c2ccc(C#N)cc2Nc2nccnc2C)nc(C(N)=O)c1N] | 751 |
| 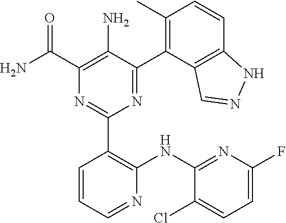 [SMILES: Cc1ccc2[nH]ncc2c1-c1nc(-c2cccnc2Nc2nc(F)ccc2Cl)nc(C(N)=O)c1N] | 752 |
| 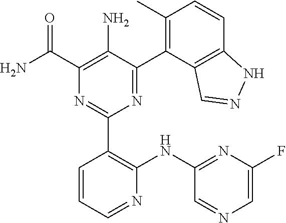 [SMILES: Cc1ccc2[nH]ncc2c1-c1nc(-c2cccnc2Nc2cncc(F)n2)nc(C(N)=O)c1N] | 753 |
| 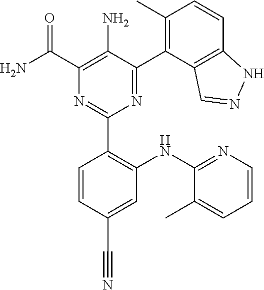 [SMILES: Cc1cccnc1Nc1cc(C#N)ccc1-c1nc(C(N)=O)c(N)c(-c2c(C)ccc3[nH]ncc23)n1] | 754 |
| 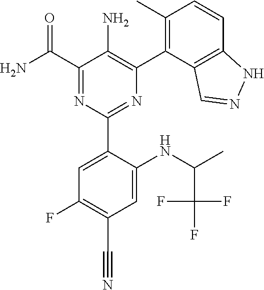 [SMILES: Cc1ccc2[nH]ncc2c1-c1nc(-c2cc(F)c(C#N)cc2NC(C)C(F)(F)F)nc(C(N)=O)c1N] | 755 |
| 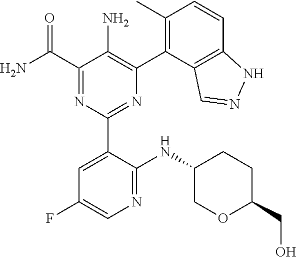 [SMILES: Cc1ccc2[nH]ncc2c1-c1nc(-c2cc(F)cnc2N[C@@H]2CC[C@@H](CO)OC2)nc(C(N)=O)c1N] | 756 |
| 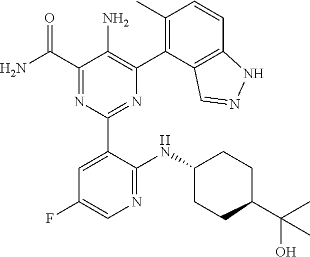 [SMILES: Cc1ccc2[nH]ncc2c1-c1nc(-c2cc(F)cnc2N[C@H]2CC[C@H](C(C)(C)O)CC2)nc(C(N)=O)c1N] | 757 |
| 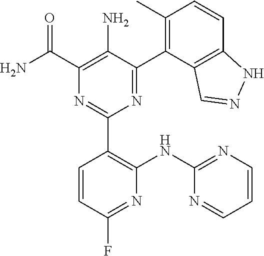 [SMILES: Cc1ccc2[nH]ncc2c1-c1nc(-c2ccc(F)nc2Nc2ncccn2)nc(C(N)=O)c1N] | 758 |
| 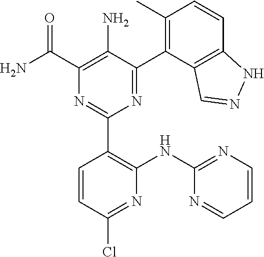 [SMILES: Cc1ccc2[nH]ncc2c1-c1nc(-c2ccc(Cl)nc2Nc2ncccn2)nc(C(N)=O)c1N] | 759 |
| 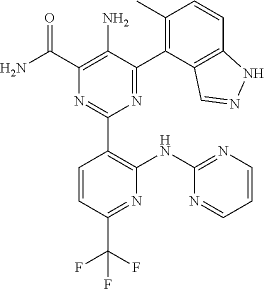 [SMILES: Cc1ccc2[nH]ncc2c1-c1nc(-c2ccc(C(F)(F)F)nc2Nc2ncccn2)nc(C(N)=O)c1N] | 760 |
| 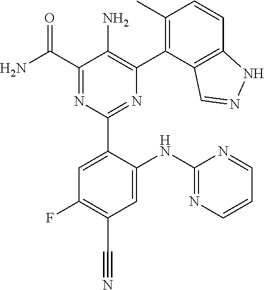 [SMILES: Cc1ccc2[nH]ncc2c1-c1nc(-c2cc(F)c(C#N)cc2Nc2ncccn2)nc(C(N)=O)c1N] | 761 |
| 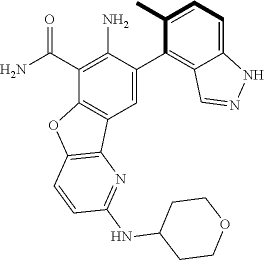 | 762 |
| 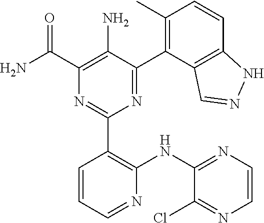 [SMILES: Cc1ccc2[nH]ncc2c1-c1nc(-c2cccnc2Nc2nccnc2Cl)nc(C(N)=O)c1N] | 763 |
| 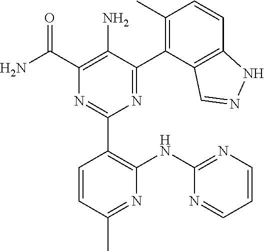 [SMILES: Cc1ccc(-c2nc(C(N)=O)c(N)c(-c3c(C)ccc4[nH]ncc34)n2)c(Nc2ncccn2)n1] | 764 |
| 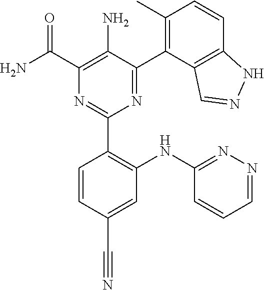 [SMILES: Cc1ccc2[nH]ncc2c1-c1nc(-c2ccc(C#N)cc2Nc2cccnn2)nc(C(N)=O)c1N] | 765 |
| 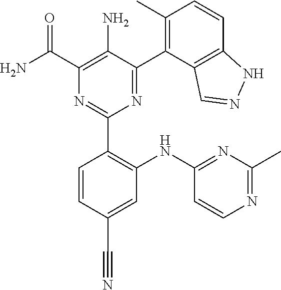 [SMILES: Cc1nccc(Nc2cc(C#N)ccc2-c2nc(C(N)=O)c(N)c(-c3c(C)ccc4[nH]ncc34)n2)n1] | 766 |
| 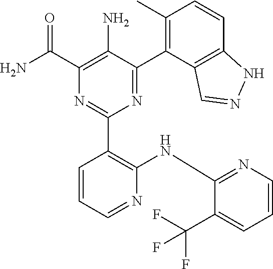 [SMILES: Cc1ccc2[nH]ncc2c1-c1nc(-c2cccnc2Nc2ncccc2C(F)(F)F)nc(C(N)=O)c1N] | 767 |
| 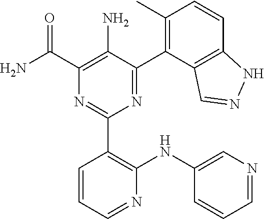 [SMILES: Cc1ccc2[nH]ncc2c1-c1nc(-c2cccnc2Nc2cccnc2)nc(C(N)=O)c1N] | 768 |
| 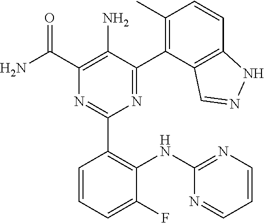 [SMILES: Cc1ccc2[nH]ncc2c1-c1nc(-c2cccc(F)c2Nc2ncccn2)nc(C(N)=O)c1N] | 769 |
| 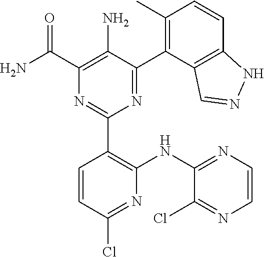 [SMILES: Cc1ccc2[nH]ncc2c1-c1nc(-c2ccc(Cl)nc2Nc2nccnc2Cl)nc(C(N)=O)c1N] | 770 |
| 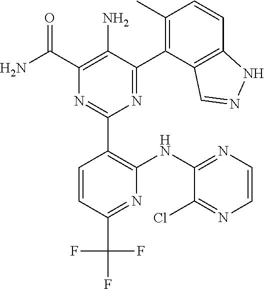 [SMILES: Cc1ccc2[nH]ncc2c1-c1nc(-c2ccc(C(F)(F)F)nc2Nc2nccnc2Cl)nc(C(N)=O)c1N] | 771 |
| 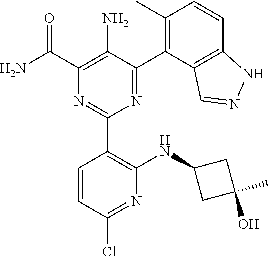 [SMILES: Cc1ccc2[nH]ncc2c1-c1nc(-c2ccc(Cl)nc2N[C@H]2C[C@@](C)(O)C2)nc(C(N)=O)c1N] | 772 |
| 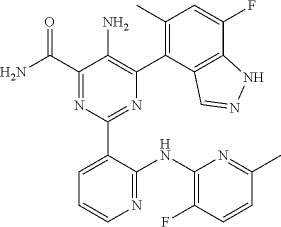 [SMILES: Cc1ccc(F)c(Nc2ncccc2-c2nc(C(N)=O)c(N)c(-c3c(C)cc(F)c4[nH]ncc34)n2)n1] | 773 |
| 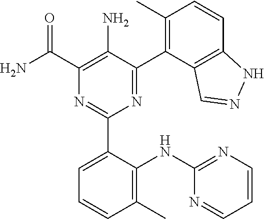 [SMILES: Cc1cccc(-c2nc(C(N)=O)c(N)c(-c3c(C)ccc4[nH]ncc34)n2)c1Nc1ncccn1] | 774 |
| 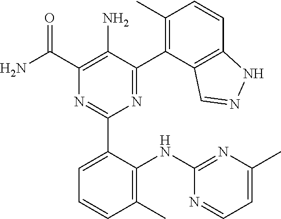 [SMILES: Cc1ccnc(Nc2c(C)cccc2-c2nc(C(N)=O)c(N)c(-c3c(C)ccc4[nH]ncc34)n2)n1] | 775 |
| 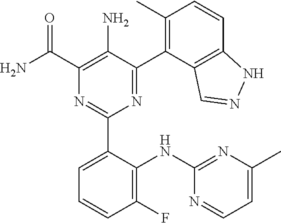 [SMILES: Cc1ccnc(Nc2c(F)cccc2-c2nc(C(N)=O)c(N)c(-c3c(C)ccc4[nH]ncc34)n2)n1] | 776 |
| 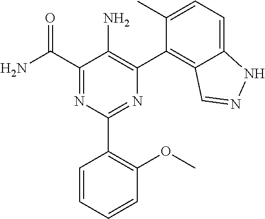 [SMILES: COc1ccccc1-c1nc(C(N)=O)c(N)c(-c2c(C)ccc3[nH]ncc23)n1] | 777 |
| 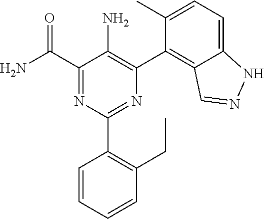 [SMILES: CCc1ccccc1-c1nc(C(N)=O)c(N)c(-c2c(C)ccc3[nH]ncc23)n1] | 778 |
| 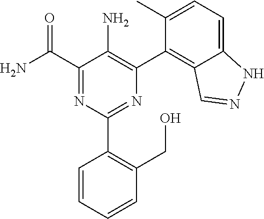 [SMILES: Cc1ccc2[nH]ncc2c1-c1nc(-c2ccccc2CO)nc(C(N)=O)c1N] | 779 |
| 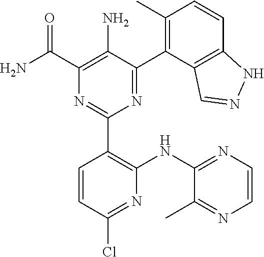 [SMILES: Cc1ccc2[nH]ncc2c1-c1nc(-c2ccc(Cl)nc2Nc2nccnc2C)nc(C(N)=O)c1N] | 780 |
| 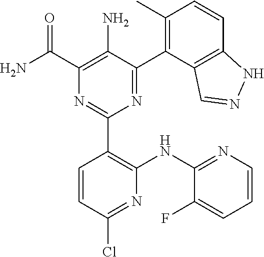 [SMILES: Cc1ccc2[nH]ncc2c1-c1nc(-c2ccc(Cl)nc2Nc2ncccc2F)nc(C(N)=O)c1N] | 781 |
| 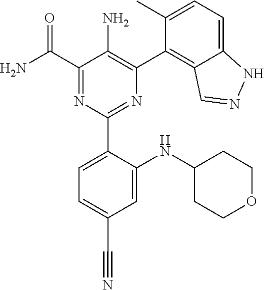 [SMILES: Cc1ccc2[nH]ncc2c1-c1nc(-c2ccc(C#N)cc2NC2CCOCC2)nc(C(N)=O)c1N] | 782 |
| 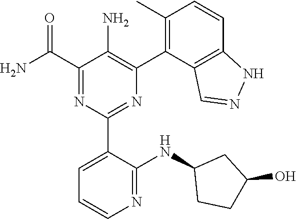 [SMILES: Cc1ccc2[nH]ncc2c1-c1nc(-c2cccnc2N[C@@H]2CC[C@H](O)C2)nc(C(N)=O)c1N] | 783 |
| 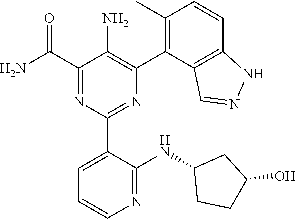 [SMILES: Cc1ccc2[nH]ncc2c1-c1nc(-c2cccnc2N[C@H]2CC[C@@H](O)C2)nc(C(N)=O)c1N] | 784 |
| 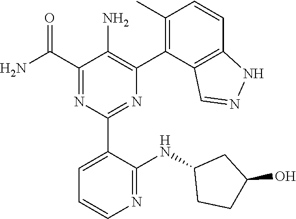 [SMILES: Cc1ccc2[nH]ncc2c1-c1nc(-c2cccnc2N[C@H]2CC[C@H](O)C2)nc(C(N)=O)c1N] | 785 |
| 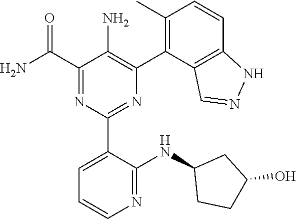 [SMILES: Cc1ccc2[nH]ncc2c1-c1nc(-c2cccnc2N[C@@H]2CC[C@@H](O)C2)nc(C(N)=O)c1N] | 786 |
| 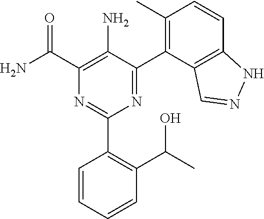 [SMILES: Cc1ccc2[nH]ncc2c1-c1nc(-c2ccccc2C(C)O)nc(C(N)=O)c1N] | 787 |
| 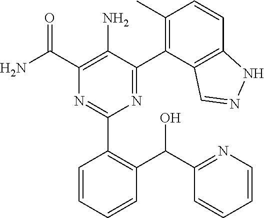 [SMILES: Cc1ccc2[nH]ncc2c1-c1nc(-c2ccccc2C(O)c2ccccn2)nc(C(N)=O)c1N] | 788 |
| 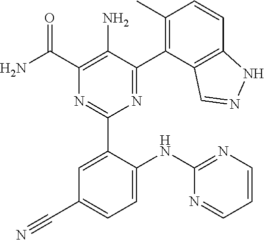 [SMILES: Cc1ccc2[nH]ncc2c1-c1nc(-c2cc(C#N)ccc2Nc2ncccn2)nc(C(N)=O)c1N] | 789 |
| 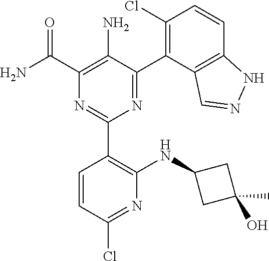 [SMILES: C[C@]1(O)C[C@@H](Nc2nc(Cl)ccc2-c2nc(C(N)=O)c(N)c(-c3c(Cl)ccc4[nH]ncc34)n2)C1] | 790 |
| 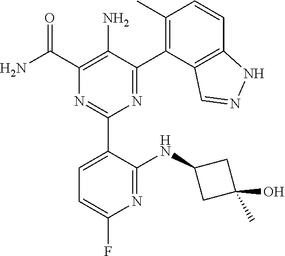 [SMILES: Cc1ccc2[nH]ncc2c1-c1nc(-c2ccc(F)nc2N[C@H]2C[C@@](C)(O)C2)nc(C(N)=O)c1N] | 791 |
| 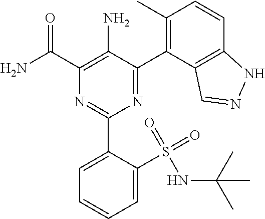 [SMILES: Cc1ccc2[nH]ncc2c1-c1nc(-c2ccccc2S(=O)(=O)NC(C)(C)C)nc(C(N)=O)c1N] | 792 |
| 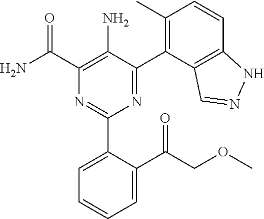 [SMILES: COCC(=O)c1ccccc1-c1nc(C(N)=O)c(N)c(-c2c(C)ccc3[nH]ncc23)n1] | 793 |
| 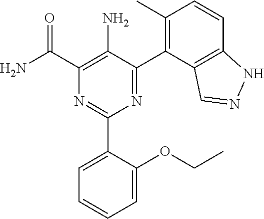 [SMILES: CCOc1ccccc1-c1nc(C(N)=O)c(N)c(-c2c(C)ccc3[nH]ncc23)n1] | 794 |
| 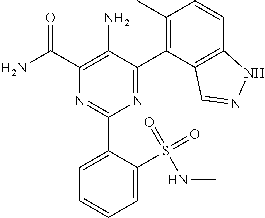 [SMILES: CNS(=O)(=O)c1ccccc1-c1nc(C(N)=O)c(N)c(-c2c(C)ccc3[nH]ncc23)n1] | 795 |
| 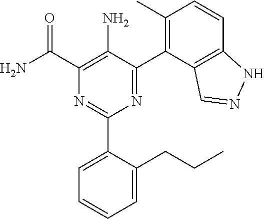 [SMILES: CCCc1ccccc1-c1nc(C(N)=O)c(N)c(-c2c(C)ccc3[nH]ncc23)n1] | 796 |
| 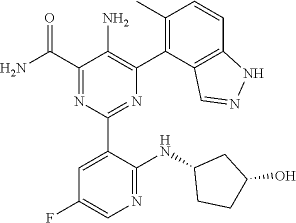 [SMILES: Cc1ccc2[nH]ncc2c1-c1nc(-c2cc(F)cnc2N[C@H]2CC[C@@H](O)C2)nc(C(N)=O)c1N] | 797 |
| 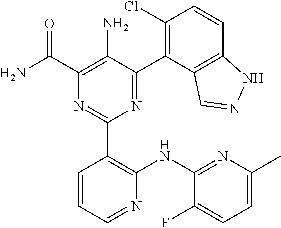 [SMILES: Cc1ccc(F)c(Nc2ncccc2-c2nc(C(N)=O)c(N)c(-c3c(Cl)ccc4[nH]ncc34)n2)n1] | 798 |
| 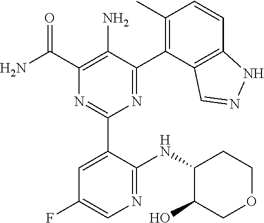 [SMILES: Cc1ccc2[nH]ncc2c1-c1nc(-c2cc(F)cnc2N[C@@H]2CCOC[C@H]2O)nc(C(N)=O)c1N] | 799 |
| 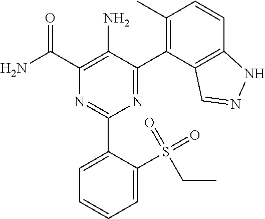 [SMILES: CCS(=O)(=O)c1ccccc1-c1nc(C(N)=O)c(N)c(-c2c(C)ccc3[nH]ncc23)n1] | 800 |
| 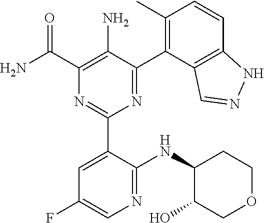 [SMILES: Cc1ccc2[nH]ncc2c1-c1nc(-c2cc(F)cnc2N[C@H]2CCOC[C@@H]2O)nc(C(N)=O)c1N] | 801 |
| 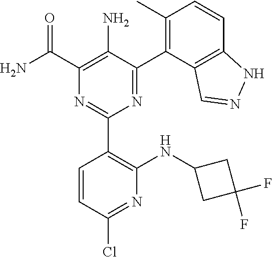 [SMILES: Cc1ccc2[nH]ncc2c1-c1nc(-c2ccc(Cl)nc2NC2CC(F)(F)C2)nc(C(N)=O)c1N] | 802 |
| 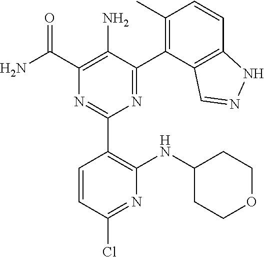 [SMILES: Cc1ccc2[nH]ncc2c1-c1nc(-c2ccc(Cl)nc2NC2CCOCC2)nc(C(N)=O)c1N] | 803 |
| 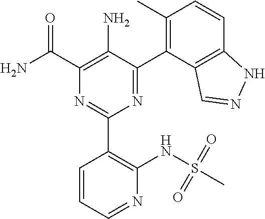 [SMILES: Cc1ccc2[nH]ncc2c1-c1nc(-c2cccnc2NS(C)(=O)=O)nc(C(N)=O)c1N] | 804 |
| 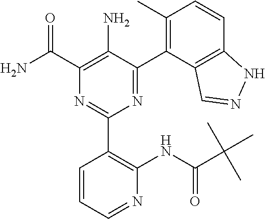 [SMILES: Cc1ccc2[nH]ncc2c1-c1nc(-c2cccnc2NC(=O)C(C)(C)C)nc(C(N)=O)c1N] | 805 |
| 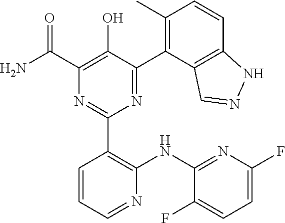 [SMILES: Cc1ccc2[nH]ncc2c1-c1nc(-c2cccnc2Nc2nc(F)ccc2F)nc(C(N)=O)c1O] | 806 |
| 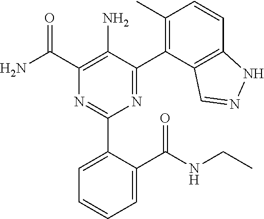 [SMILES: CCNC(=O)c1ccccc1-c1nc(C(N)=O)c(N)c(-c2c(C)ccc3[nH]ncc23)n1] | 807 |
| 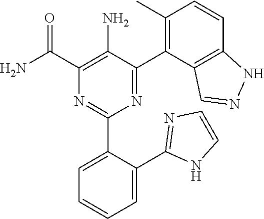 [SMILES: Cc1ccc2[nH]ncc2c1-c1nc(-c2ccccc2-c2ncc[nH]2)nc(C(N)=O)c1N] | 808 |
| 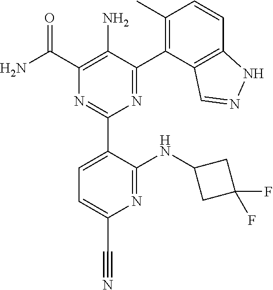 [SMILES: Cc1ccc2[nH]ncc2c1-c1nc(-c2ccc(C#N)nc2NC2CC(F)(F)C2)nc(C(N)=O)c1N] | 809 |
| 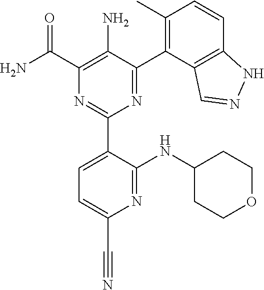 [SMILES: Cc1ccc2[nH]ncc2c1-c1nc(-c2ccc(C#N)nc2NC2CCOCC2)nc(C(N)=O)c1N] | 810 |
| 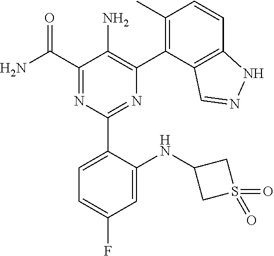 [SMILES: Cc1ccc2[nH]ncc2c1-c1nc(-c2ccc(F)cc2NC2CS(=O)(=O)C2)nc(C(N)=O)c1N] | 811 |
| 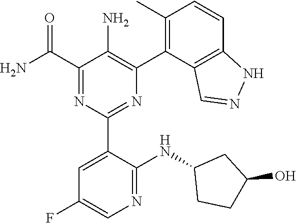 [SMILES: Cc1ccc2[nH]ncc2c1-c1nc(-c2cc(F)cnc2N[C@H]2CC[C@H](O)C2)nc(C(N)=O)c1N] | 812 |
| 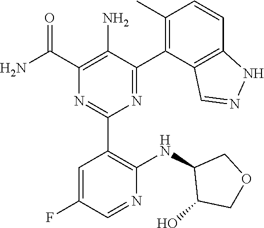 [SMILES: Cc1ccc2[nH]ncc2c1-c1nc(-c2cc(F)cnc2N[C@H]2COC[C@@H]2O)nc(C(N)=O)c1N] | 813 |
| 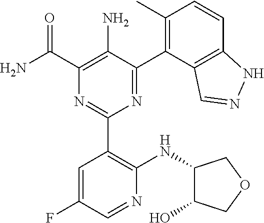 [SMILES: Cc1ccc2[nH]ncc2c1-c1nc(-c2cc(F)cnc2N[C@@H]2COC[C@@H]2O)nc(C(N)=O)c1N] | 814 |
| 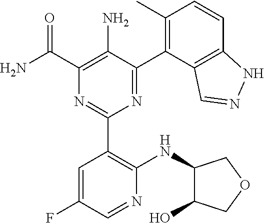 [SMILES: Cc1ccc2[nH]ncc2c1-c1nc(-c2cc(F)cnc2N[C@H]2COC[C@H]2O)nc(C(N)=O)c1N] | 815 |
| 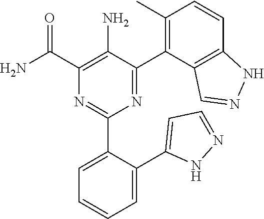 [SMILES: Cc1ccc2[nH]ncc2c1-c1nc(-c2ccccc2-c2ccn[nH]2)nc(C(N)=O)c1N] | 816 |
| 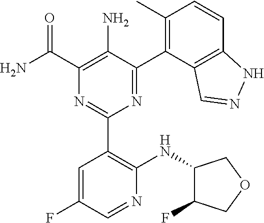 [SMILES: Cc1ccc2[nH]ncc2c1-c1nc(-c2cc(F)cnc2N[C@@H]2COC[C@H]2F)nc(C(N)=O)c1N] | 817 |
| 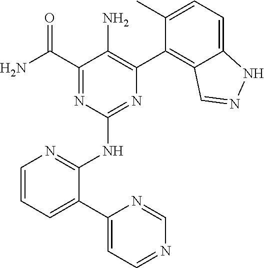 [SMILES: Cc1ccc2[nH]ncc2c1-c1nc(Nc2ncccc2-c2ccncn2)nc(C(N)=O)c1N] | 818 |
| 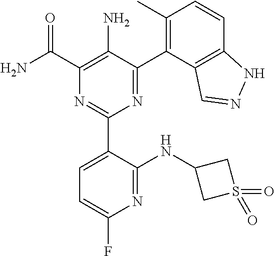 [SMILES: Cc1ccc2[nH]ncc2c1-c1nc(-c2ccc(F)nc2NC2CS(=O)(=O)C2)nc(C(N)=O)c1N] | 819 |
| 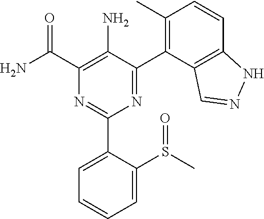 [SMILES: Cc1ccc2[nH]ncc2c1-c1nc(-c2ccccc2S(C)=O)nc(C(N)=O)c1N] | 820 |
| 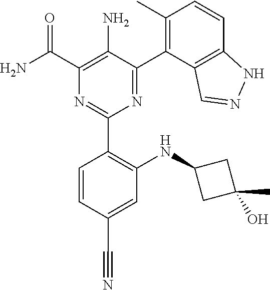 [SMILES: Cc1ccc2[nH]ncc2c1-c1nc(-c2ccc(C#N)cc2N[C@H]2C[C@](C)(O)C2)nc(C(N)=O)c1N] | 821 |
| 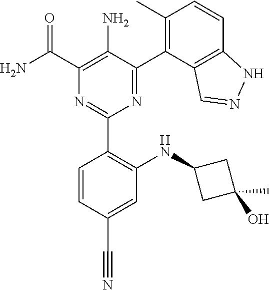 [SMILES: Cc1ccc2[nH]ncc2c1-c1nc(-c2ccc(C#N)cc2N[C@H]2C[C@@](C)(O)C2)nc(C(N)=O)c1N] | 822 |
| 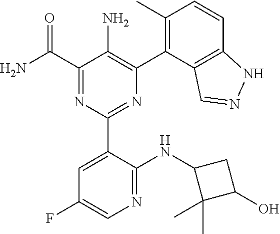 [SMILES: Cc1ccc2[nH]ncc2c1-c1nc(-c2cc(F)cnc2NC2CC(O)C2(C)C)nc(C(N)=O)c1N] | 823 |
| 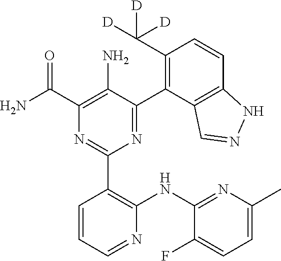 [SMILES: [2H]C([2H])([2H])c1ccc2[nH]ncc2c1-c1nc(-c2cccnc2Nc2nc(C)ccc2F)nc(C(N)=O)c1N] | 824 |
| 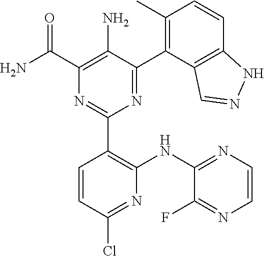 [SMILES: Cc1ccc2[nH]ncc2c1-c1nc(-c2ccc(Cl)nc2Nc2nccnc2F)nc(C(N)=O)c1N] | 825 |
| 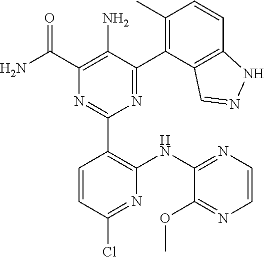 [SMILES: COc1nccnc1Nc1nc(Cl)ccc1-c1nc(C(N)=O)c(N)c(-c2c(C)ccc3[nH]ncc23)n1] | 826 |
| 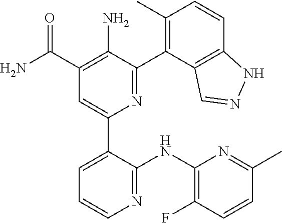 [SMILES: Cc1ccc(F)c(Nc2ncccc2-c2cc(C(N)=O)c(N)c(-c3c(C)ccc4[nH]ncc34)n2)n1] | 827 |
| 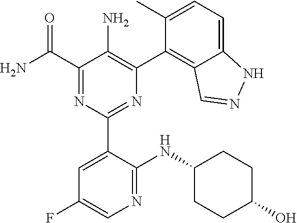 [SMILES: Cc1ccc2[nH]ncc2c1-c1nc(-c2cc(F)cnc2N[C@H]2CC[C@@H](O)CC2)nc(C(N)=O)c1N] | 828 |
| 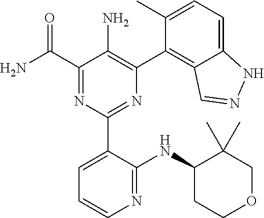 [SMILES: Cc1ccc2[nH]ncc2c1-c1nc(-c2cccnc2N[C@@H]2CCOCC2(C)C)nc(C(N)=O)c1N] | 829 |
| 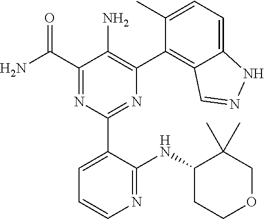 [SMILES: Cc1ccc2[nH]ncc2c1-c1nc(-c2cccnc2N[C@H]2CCOCC2(C)C)nc(C(N)=O)c1N] | 830 |
| 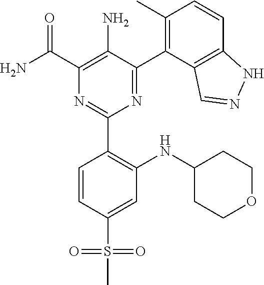 [SMILES: Cc1ccc2[nH]ncc2c1-c1nc(-c2ccc(S(C)(=O)=O)cc2NC2CCOCC2)nc(C(N)=O)c1N] | 831 |
| 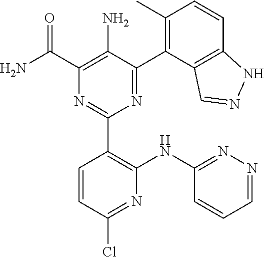 [SMILES: Cc1ccc2[nH]ncc2c1-c1nc(-c2ccc(Cl)nc2Nc2cccnn2)nc(C(N)=O)c1N] | 832 |
| 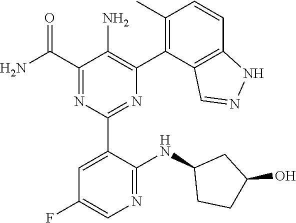 [SMILES: Cc1ccc2[nH]ncc2c1-c1nc(-c2cc(F)cnc2N[C@@H]2CC[C@H](O)C2)nc(C(N)=O)c1N] | 833 |
| 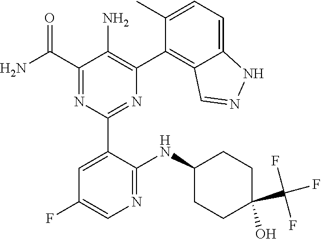 [SMILES: Cc1ccc2[nH]ncc2c1-c1nc(-c2cc(F)cnc2N[C@H]2CC[C@@](O)(C(F)(F)F)CC2)nc(C(N)=O)c1N] | 834 |
| 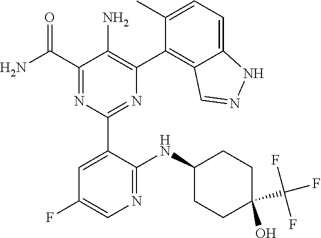 [SMILES: Cc1ccc2[nH]ncc2c1-c1nc(-c2cc(F)cnc2N[C@H]2CC[C@](O)(C(F)(F)F)CC2)nc(C(N)=O)c1N] | 835 |
| 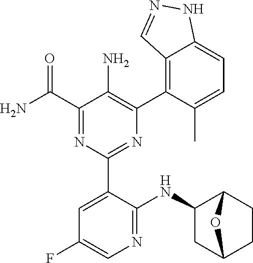 [SMILES: Cc1ccc2[nH]ncc2c1-c1nc(-c2cc(F)cnc2N[C@@H]2C[C@H]3CC[C@@H]2O3)nc(C(N)=O)c1N] | 836 |
| 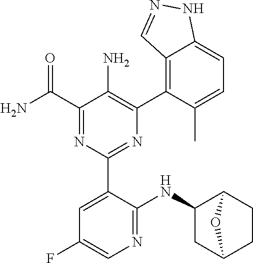 [SMILES: Cc1ccc2[nH]ncc2c1-c1nc(-c2cc(F)cnc2N[C@@H]2C[C@@H]3CC[C@H]2O3)nc(C(N)=O)c1N] | 837 |
| 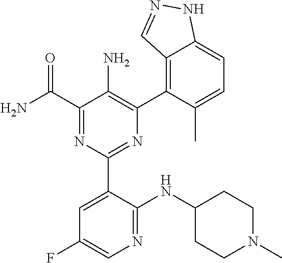 [SMILES: Cc1ccc2[nH]ncc2c1-c1nc(-c2cc(F)cnc2NC2CCN(C)CC2)nc(C(N)=O)c1N] | 838 |
| 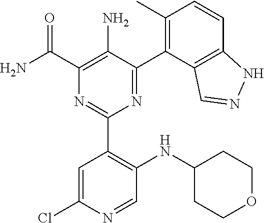 [SMILES: Cc1ccc2[nH]ncc2c1-c1nc(-c2cc(Cl)ncc2NC2CCOCC2)nc(C(N)=O)c1N] | 839 |
| 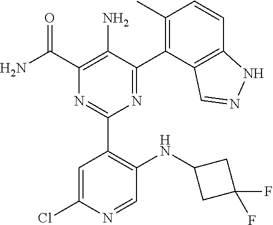 [SMILES: Cc1ccc2[nH]ncc2c1-c1nc(-c2cc(Cl)ncc2NC2CC(F)(F)C2)nc(C(N)=O)c1N] | 840 |
| 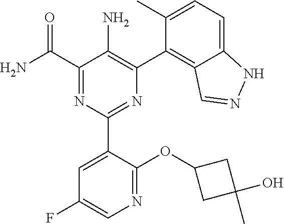 [SMILES: Cc1ccc2[nH]ncc2c1-c1nc(-c2cc(F)cnc2OC2CC(C)(O)C2)nc(C(N)=O)c1N] | 841 |
| 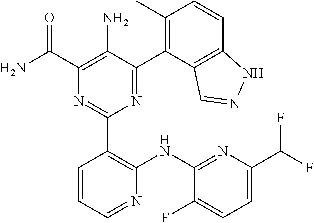 [SMILES: Cc1ccc2[nH]ncc2c1-c1nc(-c2cccnc2Nc2nc(C(F)F)ccc2F)nc(C(N)=O)c1N] | 842 |
| 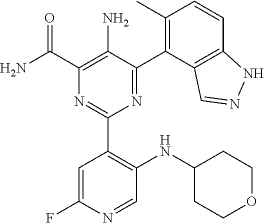 [SMILES: Cc1ccc2[nH]ncc2c1-c1nc(-c2cc(F)ncc2NC2CCOCC2)nc(C(N)=O)c1N] | 843 |
| 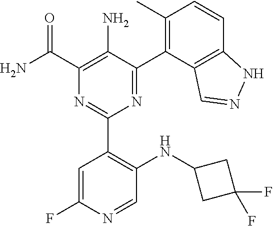 [SMILES: Cc1ccc2[nH]ncc2c1-c1nc(-c2cc(F)ncc2NC2CC(F)(F)C2)nc(C(N)=O)c1N] | 844 |
| 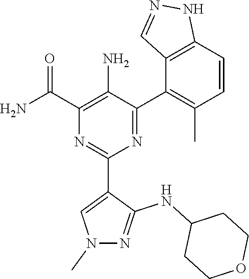 [SMILES: Cc1ccc2[nH]ncc2c1-c1nc(-c2cn(C)nc2NC2CCOCC2)nc(C(N)=O)c1N] | 845 |
| 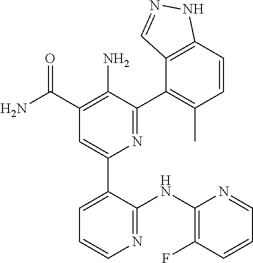 [SMILES: Cc1ccc2[nH]ncc2c1-c1nc(-c2cccnc2Nc2ncccc2F)cc(C(N)=O)c1N] | 846 |
| 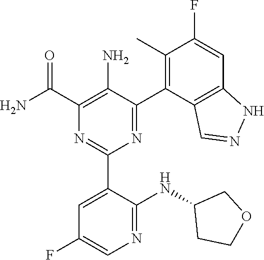 [SMILES: Cc1c(F)cc2[nH]ncc2c1-c1nc(-c2cc(F)cnc2N[C@H]2CCOC2)nc(C(N)=O)c1N] | 847 |
| 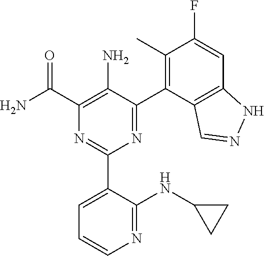 [SMILES: Cc1c(F)cc2[nH]ncc2c1-c1nc(-c2cccnc2NC2CC2)nc(C(N)=O)c1N] | 848 |
| 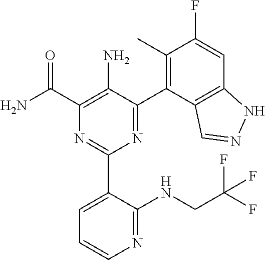 [SMILES: Cc1c(F)cc2[nH]ncc2c1-c1nc(-c2cccnc2NCC(F)(F)F)nc(C(N)=O)c1N] | 849 |
| 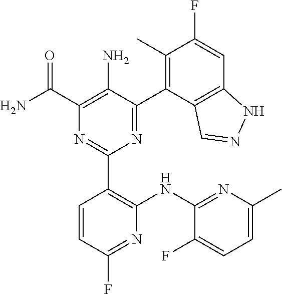 [SMILES: Cc1ccc(F)c(Nc2nc(F)ccc2-c2nc(C(N)=O)c(N)c(-c3c(C)c(F)cc4[nH]ncc34)n2)n1] | 850 |
| 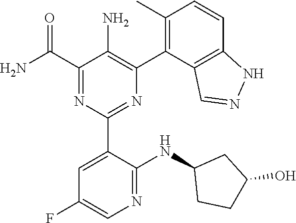 [SMILES: Cc1ccc2[nH]ncc2c1-c1nc(-c2cc(F)cnc2N[C@@H]2CC[C@@H](O)C2)nc(C(N)=O)c1N] | 851 |
| 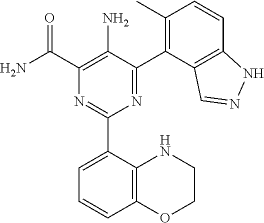 [SMILES: Cc1ccc2[nH]ncc2c1-c1nc(-c2cccc3c2NCCO3)nc(C(N)=O)c1N] | 852 |
| 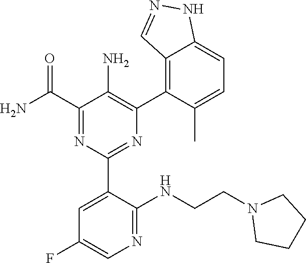 [SMILES: Cc1ccc2[nH]ncc2c1-c1nc(-c2cc(F)cnc2NCCN2CCCC2)nc(C(N)=O)c1N] | 853 |
| 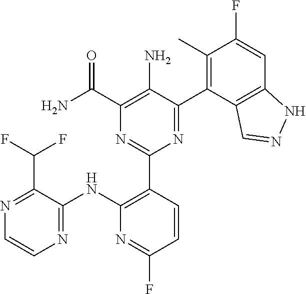 [SMILES: Cc1c(F)cc2[nH]ncc2c1-c1nc(-c2ccc(F)nc2Nc2nccnc2C(F)F)nc(C(N)=O)c1N] | 854 |
| 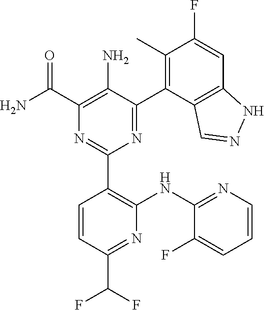 [SMILES: Cc1c(F)cc2[nH]ncc2c1-c1nc(-c2ccc(C(F)F)nc2Nc2ncccc2F)nc(C(N)=O)c1N] | 855 |
| 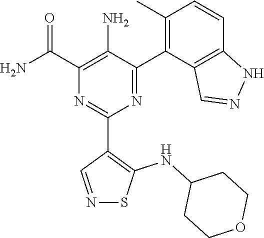 [SMILES: Cc1ccc2[nH]ncc2c1-c1nc(-c2cnsc2NC2CCOCC2)nc(C(N)=O)c1N] | 856 |
| 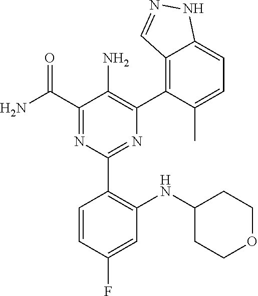 [SMILES: Cc1ccc2[nH]ncc2c1-c1nc(-c2ccc(F)cc2NC2CCOCC2)nc(C(N)=O)c1N] | 857 |
| 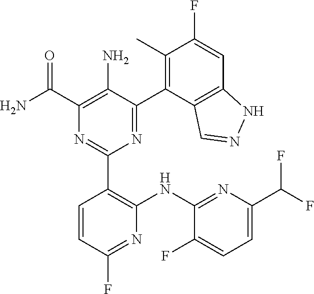 [SMILES: Cc1c(F)cc2[nH]ncc2c1-c1nc(-c2ccc(F)nc2Nc2nc(C(F)F)ccc2F)nc(C(N)=O)c1N] | 858 |
| 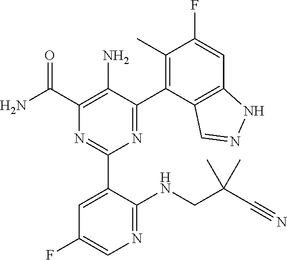 [SMILES: Cc1c(F)cc2[nH]ncc2c1-c1nc(-c2cc(F)cnc2NCC(C)(C)C#N)nc(C(N)=O)c1N] | 859 |
| 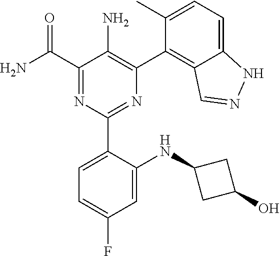 [SMILES: Cc1ccc2[nH]ncc2c1-c1nc(-c2ccc(F)cc2N[C@H]2C[C@@H](O)C2)nc(C(N)=O)c1N] | 860 |
| 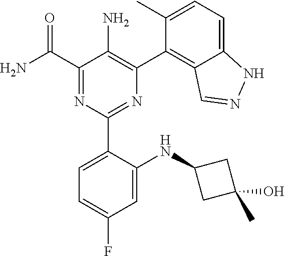 [SMILES: Cc1ccc2[nH]ncc2c1-c1nc(-c2ccc(F)cc2N[C@H]2C[C@](C)(O)C2)nc(C(N)=O)c1N] | 861 |
| 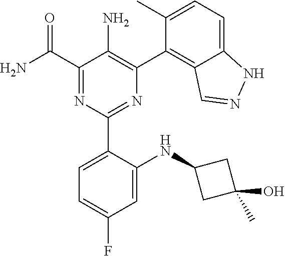 [SMILES: Cc1ccc2[nH]ncc2c1-c1nc(-c2ccc(F)cc2N[C@H]2C[C@@](C)(O)C2)nc(C(N)=O)c1N] | 862 |
| 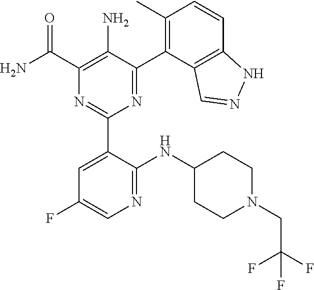 [SMILES: Cc1ccc2[nH]ncc2c1-c1nc(-c2cc(F)cnc2NC2CCN(CC(F)(F)F)CC2)nc(C(N)=O)c1N] | 863 |
| 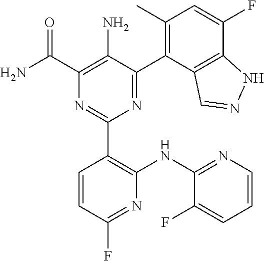 [SMILES: Cc1cc(F)c2[nH]ncc2c1-c1nc(-c2ccc(F)nc2Nc2ncccc2F)nc(C(N)=O)c1N] | 864 |
| 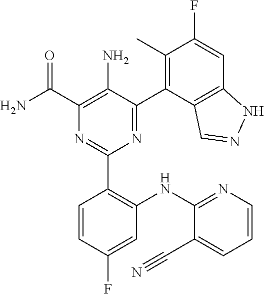 [SMILES: Cc1c(F)cc2[nH]ncc2c1-c1nc(-c2ccc(F)cc2Nc2ncccc2C#N)nc(C(N)=O)c1N] | 865 |
| 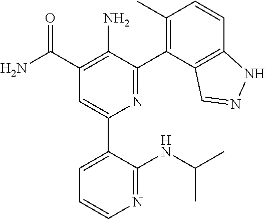 [SMILES: Cc1ccc2[nH]ncc2c1-c1nc(-c2cccnc2NC(C)C)cc(C(N)=O)c1N] | 866 |
| 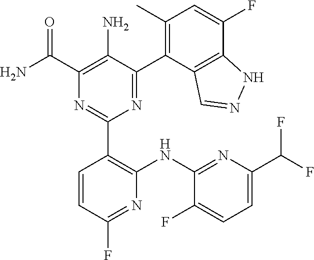 [SMILES: Cc1cc(F)c2[nH]ncc2c1-c1nc(-c2ccc(F)nc2Nc2nc(C(F)F)ccc2F)nc(C(N)=O)c1N] | 867 |
| 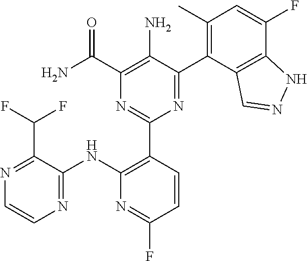 [SMILES: Cc1cc(F)c2[nH]ncc2c1-c1nc(-c2ccc(F)nc2Nc2nccnc2C(F)F)nc(C(N)=O)c1N] | 868 |
| 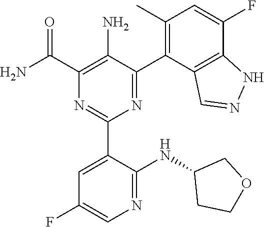 [SMILES: Cc1cc(F)c2[nH]ncc2c1-c1nc(-c2cc(F)cnc2N[C@H]2CCOC2)nc(C(N)=O)c1N] | 869 |
| 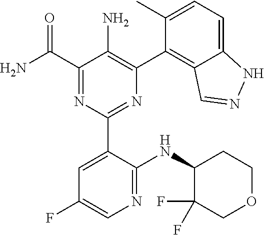 [SMILES: Cc1ccc2[nH]ncc2c1-c1nc(-c2cc(F)cnc2N[C@H]2CCOCC2(F)F)nc(C(N)=O)c1N] | 870 |
| 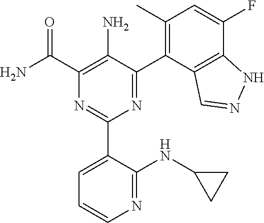 [SMILES: Cc1cc(F)c2[nH]ncc2c1-c1nc(-c2cccnc2NC2CC2)nc(C(N)=O)c1N] | 871 |
| 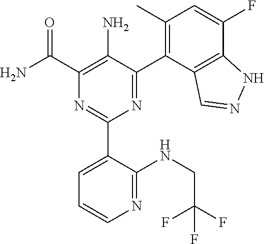 [SMILES: Cc1cc(F)c2[nH]ncc2c1-c1nc(-c2cccnc2NCC(F)(F)F)nc(C(N)=O)c1N] | 872 |
| 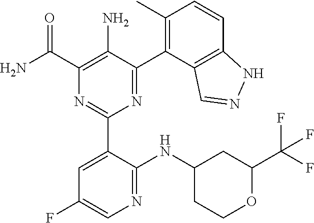 [SMILES: Cc1ccc2[nH]ncc2c1-c1nc(-c2cc(F)cnc2NC2CCOC(C(F)(F)F)C2)nc(C(N)=O)c1N] | 873 |
| 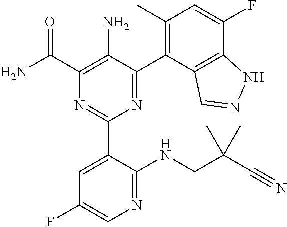 [SMILES: Cc1cc(F)c2[nH]ncc2c1-c1nc(-c2cc(F)cnc2NCC(C)(C)C#N)nc(C(N)=O)c1N] | 874 |
| 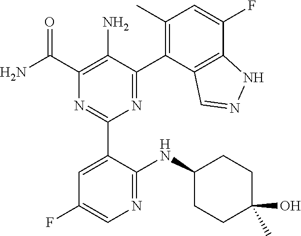 [SMILES: Cc1cc(F)c2[nH]ncc2c1-c1nc(-c2cc(F)cnc2N[C@H]2CC[C@@](C)(O)CC2)nc(C(N)=O)c1N] | 875 |
| 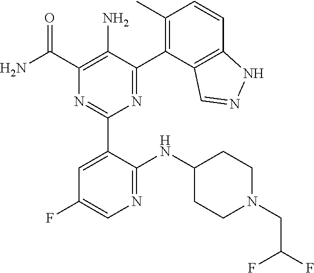 [SMILES: Cc1ccc2[nH]ncc2c1-c1nc(-c2cc(F)cnc2NC2CCN(CC(F)F)CC2)nc(C(N)=O)c1N] | 876 |
| 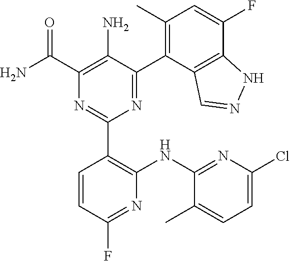 [SMILES: Cc1ccc(Cl)nc1Nc1nc(F)ccc1-c1nc(C(N)=O)c(N)c(-c2c(C)cc(F)c3[nH]ncc23)n1] | 877 |
| 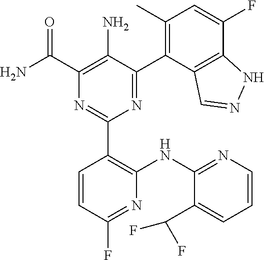 [SMILES: Cc1cc(F)c2[nH]ncc2c1-c1nc(-c2ccc(F)nc2Nc2ncccc2C(F)F)nc(C(N)=O)c1N] | 878 |
| 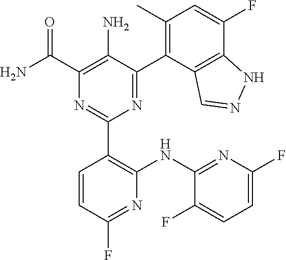 [SMILES: Cc1cc(F)c2[nH]ncc2c1-c1nc(-c2ccc(F)nc2Nc2nc(F)ccc2F)nc(C(N)=O)c1N] | 879 |
| 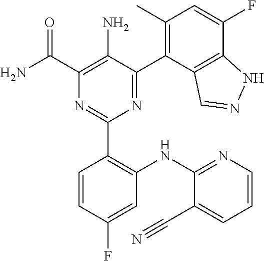 [SMILES: Cc1cc(F)c2[nH]ncc2c1-c1nc(-c2ccc(F)cc2Nc2ncccc2C#N)nc(C(N)=O)c1N] | 880 |
| 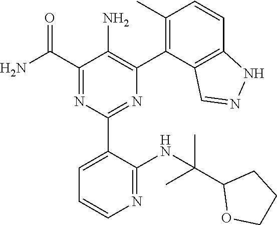 [SMILES: Cc1ccc2[nH]ncc2c1-c1nc(-c2cccnc2NC(C)(C)C2CCCO2)nc(C(N)=O)c1N] | 881 |
| 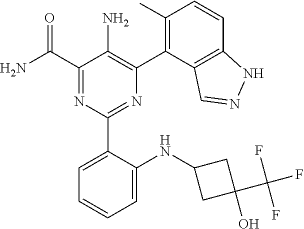 [SMILES: Cc1ccc2[nH]ncc2c1-c1nc(-c2ccccc2NC2CC(O)(C(F)(F)F)C2)nc(C(N)=O)c1N] | 882 |
| 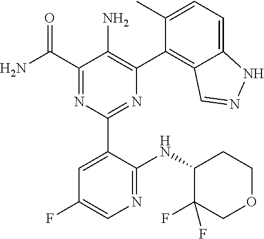 [SMILES: Cc1ccc2[nH]ncc2c1-c1nc(-c2cc(F)cnc2N[C@@H]2CCOCC2(F)F)nc(C(N)=O)c1N] | 883 |
| 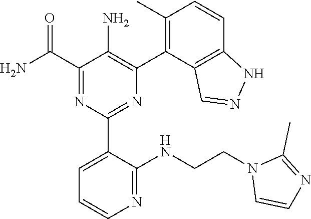 [SMILES: Cc1ccc2[nH]ncc2c1-c1nc(-c2cccnc2NCCn2ccnc2C)nc(C(N)=O)c1N] | 884 |
| 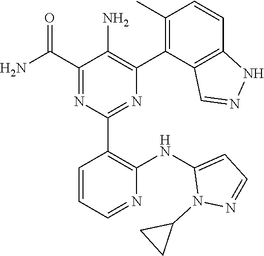 [SMILES: Cc1ccc2[nH]ncc2c1-c1nc(-c2cccnc2Nc2ccnn2C2CC2)nc(C(N)=O)c1N] | 885 |
| 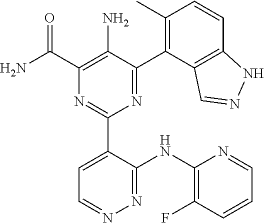 [SMILES: Cc1ccc2[nH]ncc2c1-c1nc(-c2ccnnc2Nc2ncccc2F)nc(C(N)=O)c1N] | 886 |
| 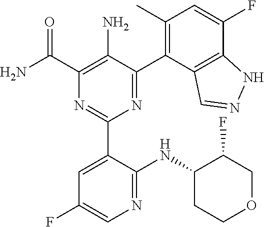 [SMILES: Cc1cc(F)c2[nH]ncc2c1-c1nc(-c2cc(F)cnc2N[C@H]2CCOC[C@H]2F)nc(C(N)=O)c1N] | 887 |
| 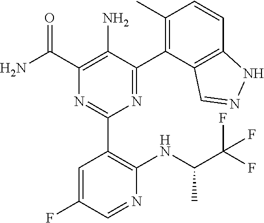 [SMILES: Cc1ccc2[nH]ncc2c1-c1nc(-c2cc(F)cnc2N[C@@H](C)C(F)(F)F)nc(C(N)=O)c1N] | 888 |
| 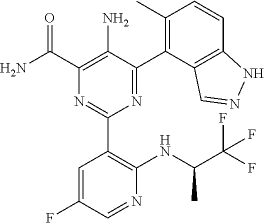 [SMILES: Cc1ccc2[nH]ncc2c1-c1nc(-c2cc(F)cnc2N[C@H](C)C(F)(F)F)nc(C(N)=O)c1N] | 889 |
| 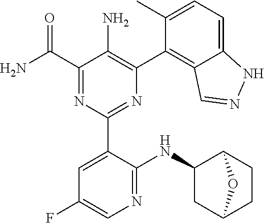 [SMILES: Cc1ccc2[nH]ncc2c1-c1nc(-c2cc(F)cnc2N[C@@H]2C[C@@H]3CC[C@H]2O3)nc(C(N)=O)c1N] | 890 |
| 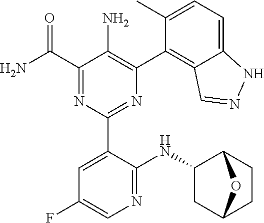 [SMILES: Cc1ccc2[nH]ncc2c1-c1nc(-c2cc(F)cnc2N[C@H]2C[C@H]3CC[C@@H]2O3)nc(C(N)=O)c1N] | 891 |
| 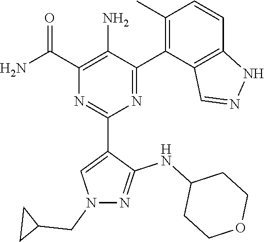 [SMILES: Cc1ccc2[nH]ncc2c1-c1nc(-c2cn(CC3CC3)nc2NC2CCOCC2)nc(C(N)=O)c1N] | 892 |
| 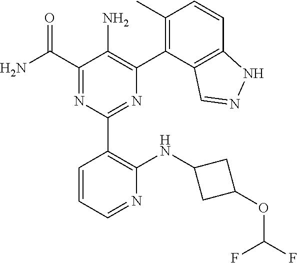 [SMILES: Cc1ccc2[nH]ncc2c1-c1nc(-c2cccnc2NC2CC(OC(F)F)C2)nc(C(N)=O)c1N] | 893 |
| 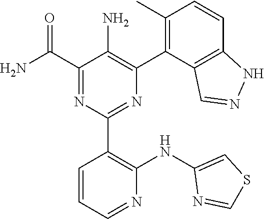 [SMILES: Cc1ccc2[nH]ncc2c1-c1nc(-c2cccnc2Nc2cscn2)nc(C(N)=O)c1N] | 894 |
| 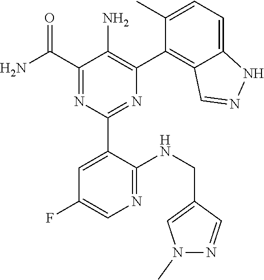 [SMILES: Cc1ccc2[nH]ncc2c1-c1nc(-c2cc(F)cnc2NCc2cnn(C)c2)nc(C(N)=O)c1N] | 895 |
| 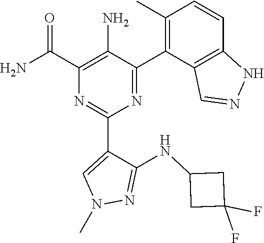 [SMILES: Cc1ccc2[nH]ncc2c1-c1nc(-c2cn(C)nc2NC2CC(F)(F)C2)nc(C(N)=O)c1N] | 896 |
| 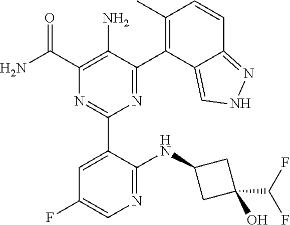 [SMILES: Cc1ccc2n[nH]cc2c1-c1nc(-c2cc(F)cnc2N[C@H]2C[C@](O)(C(F)F)C2)nc(C(N)=O)c1N] | 897 |
| 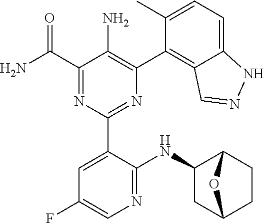 [SMILES: Cc1ccc2[nH]ncc2c1-c1nc(-c2cc(F)cnc2N[C@@H]2C[C@H]3CC[C@@H]2O3)nc(C(N)=O)c1N] | 898 |
| 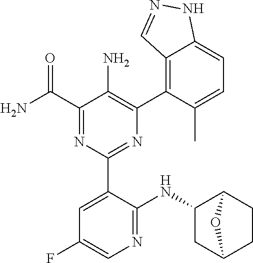 [SMILES: Cc1ccc2[nH]ncc2c1-c1nc(-c2cc(F)cnc2N[C@H]2C[C@@H]3CC[C@H]2O3)nc(C(N)=O)c1N] | 899 |
| 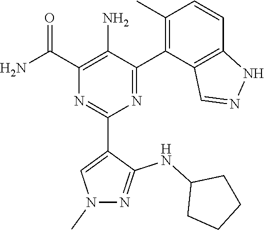 [SMILES: Cc1ccc2[nH]ncc2c1-c1nc(-c2cn(C)nc2NC2CCCC2)nc(C(N)=O)c1N] | 900 |
| 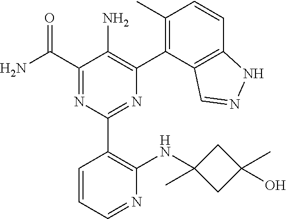 [SMILES: Cc1ccc2[nH]ncc2c1-c1nc(-c2cccnc2NC2(C)CC(C)(O)C2)nc(C(N)=O)c1N] | 901 |
| 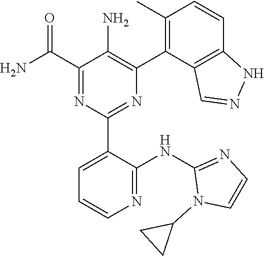 [SMILES: Cc1ccc2[nH]ncc2c1-c1nc(-c2cccnc2Nc2nccn2C2CC2)nc(C(N)=O)c1N] | 902 |
| 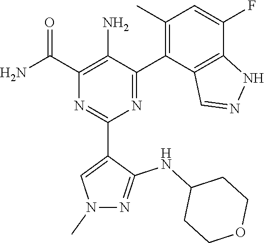 [SMILES: Cc1cc(F)c2[nH]ncc2c1-c1nc(-c2cn(C)nc2NC2CCOCC2)nc(C(N)=O)c1N] | 903 |
| 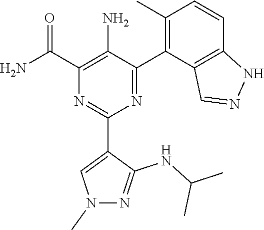 [SMILES: Cc1ccc2[nH]ncc2c1-c1nc(-c2cn(C)nc2NC(C)C)nc(C(N)=O)c1N] | 904 |
| 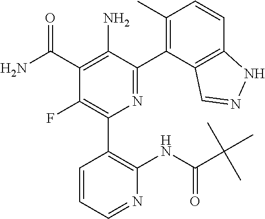 [SMILES: Cc1ccc2[nH]ncc2c1-c1nc(-c2cccnc2NC(=O)C(C)(C)C)c(F)c(C(N)=O)c1N] | 905 |
| 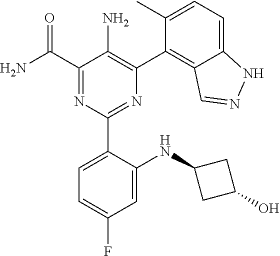 [SMILES: Cc1ccc2[nH]ncc2c1-c1nc(-c2ccc(F)cc2N[C@H]2C[C@H](O)C2)nc(C(N)=O)c1N] | 906 |
| 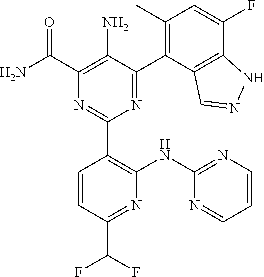 [SMILES: Cc1cc(F)c2[nH]ncc2c1-c1nc(-c2ccc(C(F)F)nc2Nc2ncccn2)nc(C(N)=O)c1N] | 907 |
| 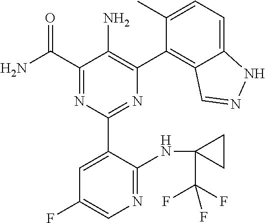 [SMILES: Cc1ccc2[nH]ncc2c1-c1nc(-c2cc(F)cnc2NC2(C(F)(F)F)CC2)nc(C(N)=O)c1N] | 908 |
| 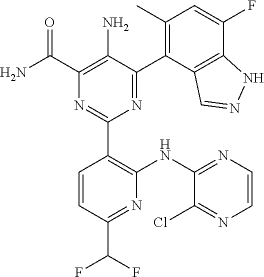 [SMILES: Cc1cc(F)c2[nH]ncc2c1-c1nc(-c2ccc(C(F)F)nc2Nc2nccnc2Cl)nc(C(N)=O)c1N] | 909 |
| 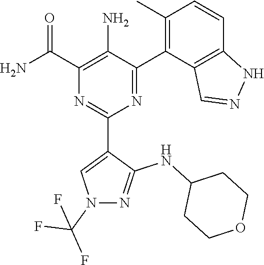 [SMILES: Cc1ccc2[nH]ncc2c1-c1nc(-c2cn(C(F)(F)F)nc2NC2CCOCC2)nc(C(N)=O)c1N] | 910 |
| 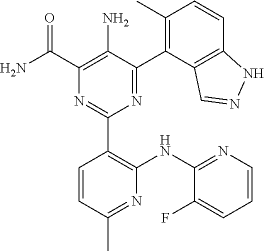 [SMILES: Cc1ccc(-c2nc(C(N)=O)c(N)c(-c3c(C)ccc4[nH]ncc34)n2)c(Nc2ncccc2F)n1] | 911 |
| 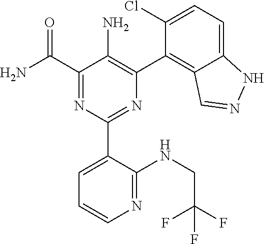 [SMILES: NC(=O)c1nc(-c2cccnc2NCC(F)(F)F)nc(-c2c(Cl)ccc3[nH]ncc23)c1N] | 912 |
| 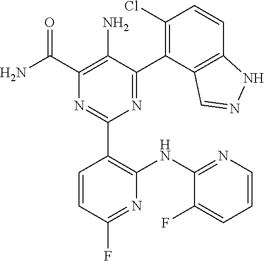 [SMILES: NC(=O)c1nc(-c2ccc(F)nc2Nc2ncccc2F)nc(-c2c(Cl)ccc3[nH]ncc23)c1N] | 913 |
| 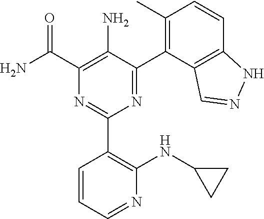 [SMILES: Cc1ccc2[nH]ncc2c1-c1nc(-c2cccnc2NC2CC2)nc(C(N)=O)c1N] | 914 |
| 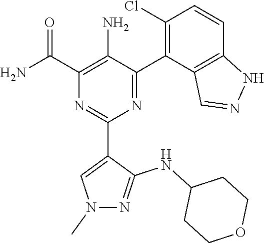 [SMILES: Cn1cc(-c2nc(C(N)=O)c(N)c(-c3c(Cl)ccc4[nH]ncc34)n2)c(NC2CCOCC2)n1] | 915 |
| 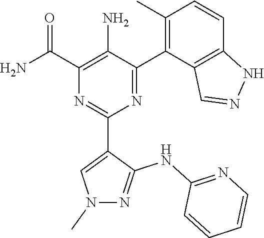 [SMILES: Cc1ccc2[nH]ncc2c1-c1nc(-c2cn(C)nc2Nc2ccccn2)nc(C(N)=O)c1N] | 916 |
| 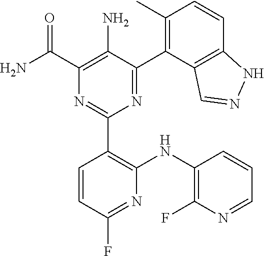 [SMILES: Cc1ccc2[nH]ncc2c1-c1nc(-c2ccc(F)nc2Nc2cccnc2F)nc(C(N)=O)c1N] | 917 |
| 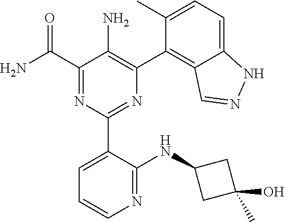 [SMILES: Cc1ccc2[nH]ncc2c1-c1nc(-c2cccnc2N[C@H]2C[C@@](C)(O)C2)nc(C(N)=O)c1N] | 918 |
| 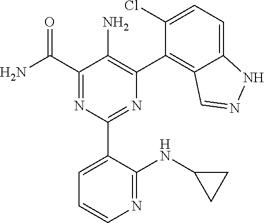 [SMILES: NC(=O)c1nc(-c2cccnc2NC2CC2)nc(-c2c(Cl)ccc3[nH]ncc23)c1N] | 919 |
| 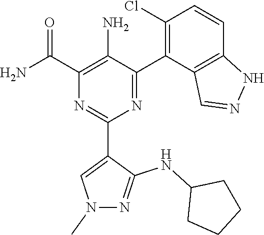 [SMILES: Cn1cc(-c2nc(C(N)=O)c(N)c(-c3c(Cl)ccc4[nH]ncc34)n2)c(NC2CCCC2)n1] | 920 |
| 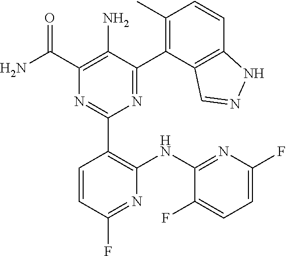 [SMILES: Cc1ccc2[nH]ncc2c1-c1nc(-c2ccc(F)nc2Nc2nc(F)ccc2F)nc(C(N)=O)c1N] | 921 |
| 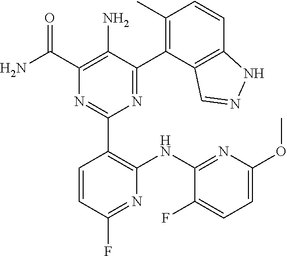 [SMILES: COc1ccc(F)c(Nc2nc(F)ccc2-c2nc(C(N)=O)c(N)c(-c3c(C)ccc4[nH]ncc34)n2)n1] | 922 |
| 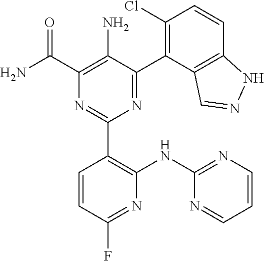 [SMILES: NC(=O)c1nc(-c2ccc(F)nc2Nc2ncccn2)nc(-c2c(Cl)ccc3[nH]ncc23)c1N] | 923 |
| 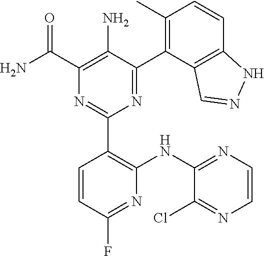 [SMILES: Cc1ccc2[nH]ncc2c1-c1nc(-c2ccc(F)nc2Nc2nccnc2Cl)nc(C(N)=O)c1N] | 924 |
| 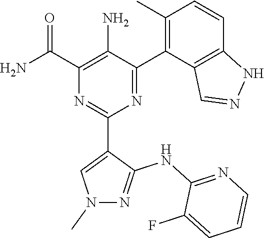 [SMILES: Cc1ccc2[nH]ncc2c1-c1nc(-c2cn(C)nc2Nc2ncccc2F)nc(C(N)=O)c1N] | 925 |
| 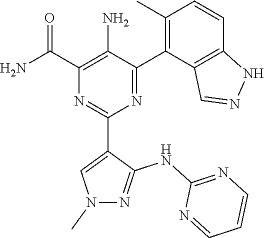 [SMILES: Cc1ccc2[nH]ncc2c1-c1nc(-c2cn(C)nc2Nc2ncccn2)nc(C(N)=O)c1N] | 926 |
| 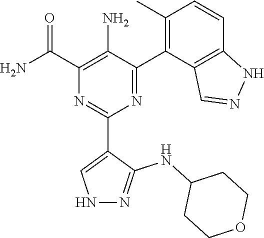 [SMILES: Cc1ccc2[nH]ncc2c1-c1nc(-c2c[nH]nc2NC2CCOCC2)nc(C(N)=O)c1N] | 927 |
| 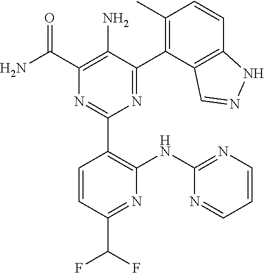 [SMILES: Cc1ccc2[nH]ncc2c1-c1nc(-c2ccc(C(F)F)nc2Nc2ncccn2)nc(C(N)=O)c1N] | 928 |
| 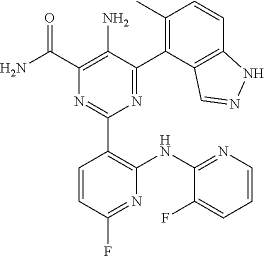 [SMILES: Cc1ccc2[nH]ncc2c1-c1nc(-c2ccc(F)nc2Nc2ncccc2F)nc(C(N)=O)c1N] | 929 |
| 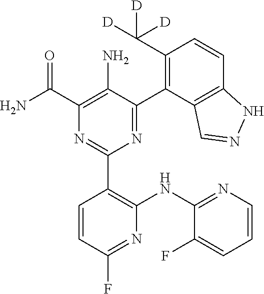 [SMILES: [2H]C([2H])([2H])c1ccc2[nH]ncc2c1-c1nc(-c2ccc(F)nc2Nc2ncccc2F)nc(C(N)=O)c1N] | 930 |
| 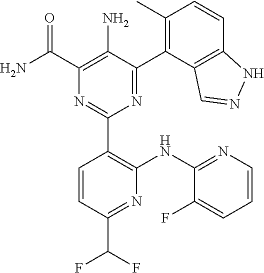 [SMILES: Cc1ccc2[nH]ncc2c1-c1nc(-c2ccc(C(F)F)nc2Nc2ncccc2F)nc(C(N)=O)c1N] | 931 |
| 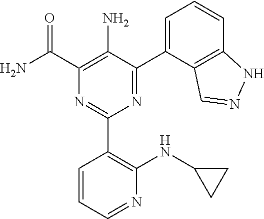 [SMILES: NC(=O)c1nc(-c2cccnc2NC2CC2)nc(-c2cccc3[nH]ncc23)c1N] | 932 |
| 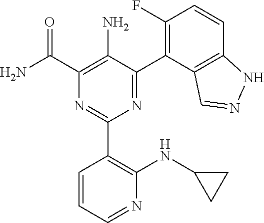 [SMILES: NC(=O)c1nc(-c2cccnc2NC2CC2)nc(-c2c(F)ccc3[nH]ncc23)c1N] | 933 |
| 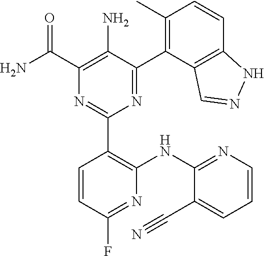 [SMILES: Cc1ccc2[nH]ncc2c1-c1nc(-c2ccc(F)nc2Nc2ncccc2C#N)nc(C(N)=O)c1N] | 934 |
| 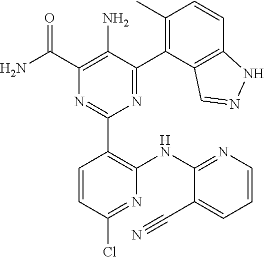 [SMILES: Cc1ccc2[nH]ncc2c1-c1nc(-c2ccc(Cl)nc2Nc2ncccc2C#N)nc(C(N)=O)c1N] | 935 |
| 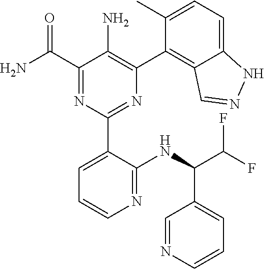 [SMILES: Cc1ccc2[nH]ncc2c1-c1nc(-c2cccnc2N[C@H](c2cccnc2)C(F)F)nc(C(N)=O)c1N] | 936 |
| 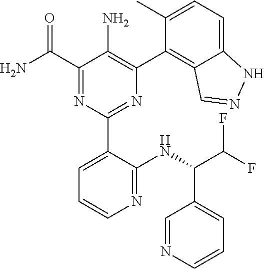 [SMILES: Cc1ccc2[nH]ncc2c1-c1nc(-c2cccnc2N[C@@H](c2cccnc2)C(F)F)nc(C(N)=O)c1N] | 937 |
| 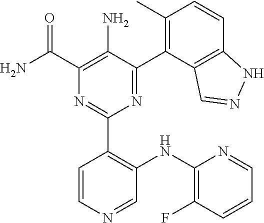 [SMILES: Cc1ccc2[nH]ncc2c1-c1nc(-c2ccncc2Nc2ncccc2F)nc(C(N)=O)c1N] | 938 |
| 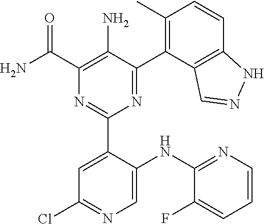 [SMILES: Cc1ccc2[nH]ncc2c1-c1nc(-c2cc(Cl)ncc2Nc2ncccc2F)nc(C(N)=O)c1N] | 939 |
| 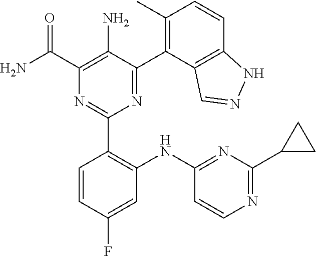 [SMILES: Cc1ccc2[nH]ncc2c1-c1nc(-c2ccc(F)cc2Nc2ccnc(C3CC3)n2)nc(C(N)=O)c1N] | 940 |
| 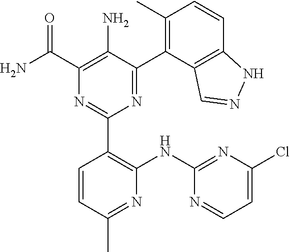 [SMILES: Cc1ccc(-c2nc(C(N)=O)c(N)c(-c3c(C)ccc4[nH]ncc34)n2)c(Nc2nccc(Cl)n2)n1] | 941 |
| 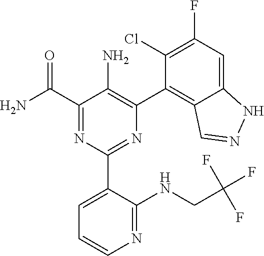 [SMILES: NC(=O)c1nc(-c2cccnc2NCC(F)(F)F)nc(-c2c(Cl)c(F)cc3[nH]ncc23)c1N] | 942 |
| 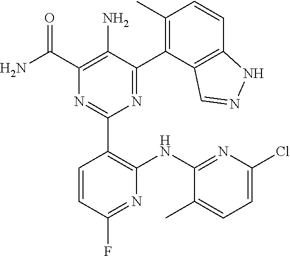 [SMILES: Cc1ccc(Cl)nc1Nc1nc(F)ccc1-c1nc(C(N)=O)c(N)c(-c2c(C)ccc3[nH]ncc23)n1] | 943 |
| 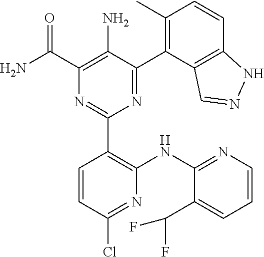 [SMILES: Cc1ccc2[nH]ncc2c1-c1nc(-c2ccc(Cl)nc2Nc2ncccc2C(F)F)nc(C(N)=O)c1N] | 944 |
| 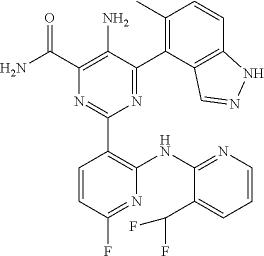 [SMILES: Cc1ccc2[nH]ncc2c1-c1nc(-c2ccc(F)nc2Nc2ncccc2C(F)F)nc(C(N)=O)c1N] | 945 |
| 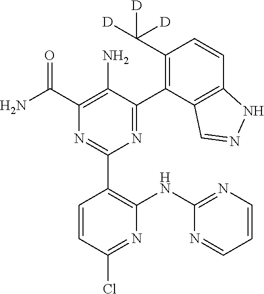 [SMILES: [2H]C([2H])([2H])c1ccc2[nH]ncc2c1-c1nc(-c2ccc(Cl)nc2Nc2ncccn2)nc(C(N)=O)c1N] | 946 |
| 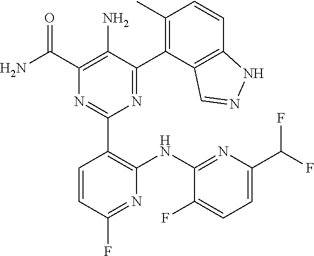 [SMILES: Cc1ccc2[nH]ncc2c1-c1nc(-c2ccc(F)nc2Nc2nc(C(F)F)ccc2F)nc(C(N)=O)c1N] | 947 |
| 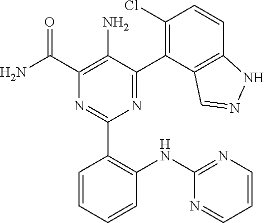 [SMILES: NC(=O)c1nc(-c2ccccc2Nc2ncccn2)nc(-c2c(Cl)ccc3[nH]ncc23)c1N] | 948 |
| 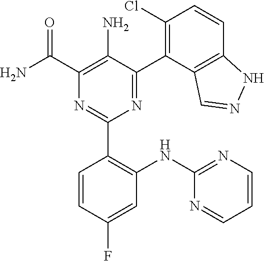 [SMILES: NC(=O)c1nc(-c2ccc(F)cc2Nc2ncccn2)nc(-c2c(Cl)ccc3[nH]ncc23)c1N] | 949 |
| 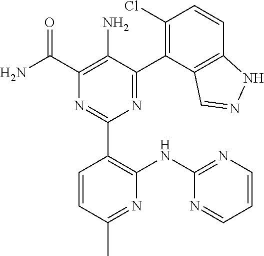 [SMILES: Cc1ccc(-c2nc(C(N)=O)c(N)c(-c3c(Cl)ccc4[nH]ncc34)n2)c(Nc2ncccn2)n1] | 950 |
| 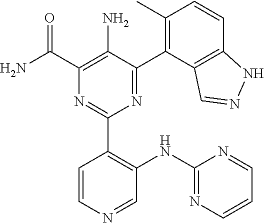 [SMILES: Cc1ccc2[nH]ncc2c1-c1nc(-c2ccncc2Nc2ncccn2)nc(C(N)=O)c1N] | 951 |
| 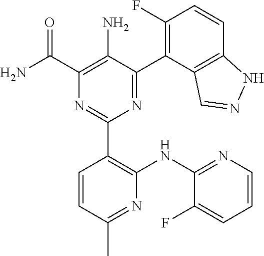 [SMILES: Cc1ccc(-c2nc(C(N)=O)c(N)c(-c3c(F)ccc4[nH]ncc34)n2)c(Nc2ncccc2F)n1] | 952 |
| 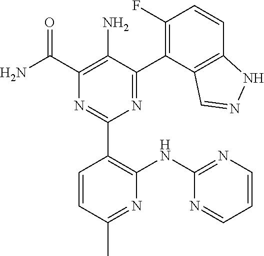 [SMILES: Cc1ccc(-c2nc(C(N)=O)c(N)c(-c3c(F)ccc4[nH]ncc34)n2)c(Nc2ncccn2)n1] | 953 |
| 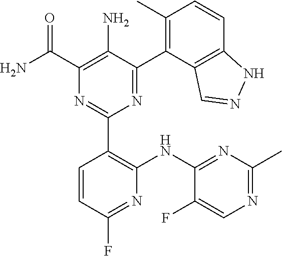 [SMILES: Cc1ncc(F)c(Nc2nc(F)ccc2-c2nc(C(N)=O)c(N)c(-c3c(C)ccc4[nH]ncc34)n2)n1] | 954 |
| 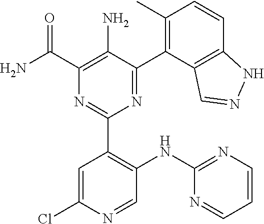 [SMILES: Cc1ccc2[nH]ncc2c1-c1nc(-c2cc(Cl)ncc2Nc2ncccn2)nc(C(N)=O)c1N] | 955 |
| 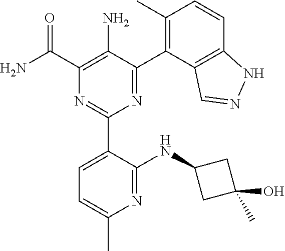 [SMILES: Cc1ccc(-c2nc(C(N)=O)c(N)c(-c3c(C)ccc4[nH]ncc34)n2)c(N[C@H]2C[C@@](C)(O)C2)n1] | 956 |
| 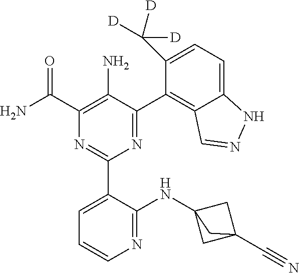 [SMILES: [2H]C([2H])([2H])c1ccc2[nH]ncc2c1-c1nc(-c2cccnc2NC23CC(C#N)(C2)C3)nc(C(N)=O)c1N] | 957 |
| 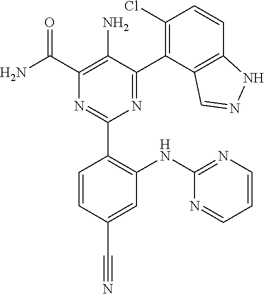 [SMILES: N#Cc1ccc(-c2nc(C(N)=O)c(N)c(-c3c(Cl)ccc4[nH]ncc34)n2)c(Nc2ncccn2)c1] | 958 |
| 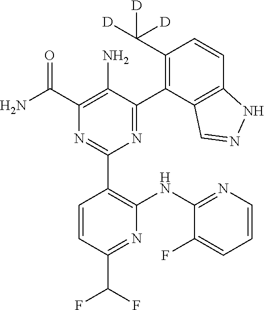 [SMILES: [2H]C([2H])([2H])c1ccc2[nH]ncc2c1-c1nc(-c2ccc(C(F)F)nc2Nc2ncccc2F)nc(C(N)=O)c1N] | 959 |
| 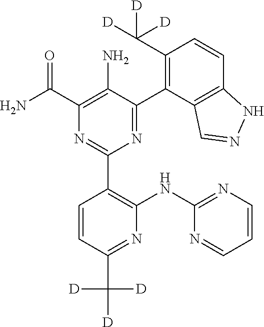 [SMILES: [2H]C([2H])([2H])c1ccc(-c2nc(C(N)=O)c(N)c(-c3c(C([2H])([2H])[2H])ccc4[nH]ncc34)n2)c(Nc2ncccn2)n1] | 960 |
| 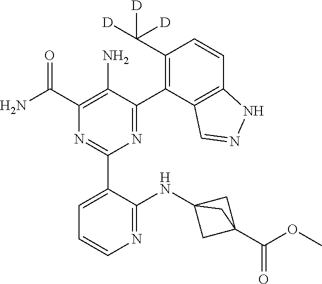 [SMILES: [2H]C([2H])([2H])c1ccc2[nH]ncc2c1-c1nc(-c2cccnc2NC23CC(C(=O)OC)(C2)C3)nc(C(N)=O)c1N] | 961 |
| 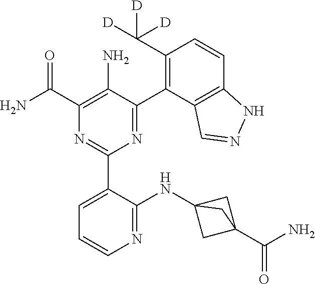 [SMILES: [2H]C([2H])([2H])c1ccc2[nH]ncc2c1-c1nc(-c2cccnc2NC23CC(C(N)=O)(C2)C3)nc(C(N)=O)c1N] | 962 |
| 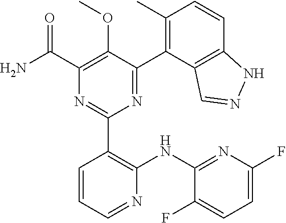 [SMILES: COc1c(C(N)=O)nc(-c2cccnc2Nc2nc(F)ccc2F)nc1-c1c(C)ccc2[nH]ncc12] | 963 |
| 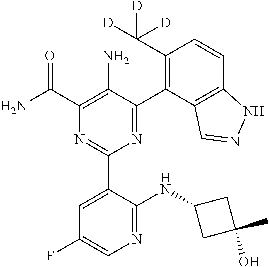 [SMILES: [2H]C([2H])([2H])c1ccc2[nH]ncc2c1-c1nc(-c2cc(F)cnc2N[C@H]2C[C@@](C)(O)C2)nc(C(N)=O)c1N] | 964 |
| 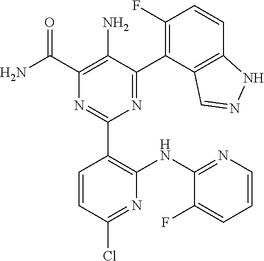 [SMILES: NC(=O)c1nc(-c2ccc(Cl)nc2Nc2ncccc2F)nc(-c2c(F)ccc3[nH]ncc23)c1N] | 965 |
| 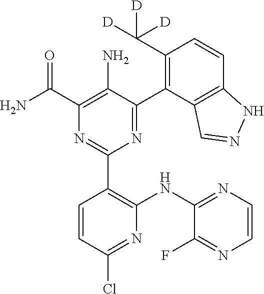 [SMILES: [2H]C([2H])([2H])c1ccc2[nH]ncc2c1-c1nc(-c2ccc(Cl)nc2Nc2nccnc2F)nc(C(N)=O)c1N] | 966 |
| 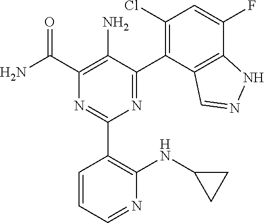 [SMILES: NC(=O)c1nc(-c2cccnc2NC2CC2)nc(-c2c(Cl)cc(F)c3[nH]ncc23)c1N] | 967 |
| 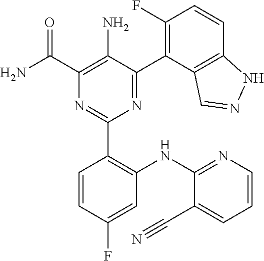 [SMILES: N#Cc1cccnc1Nc1cc(F)ccc1-c1nc(C(N)=O)c(N)c(-c2c(F)ccc3[nH]ncc23)n1] | 968 |
| 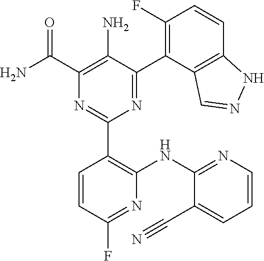 [SMILES: N#Cc1cccnc1Nc1nc(F)ccc1-c1nc(C(N)=O)c(N)c(-c2c(F)ccc3[nH]ncc23)n1] | 969 |
| 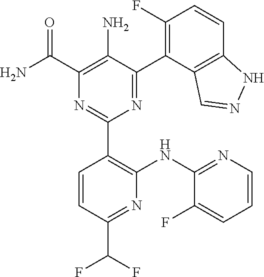 [SMILES: NC(=O)c1nc(-c2ccc(C(F)F)nc2Nc2ncccc2F)nc(-c2c(F)ccc3[nH]ncc23)c1N] | 970 |
| 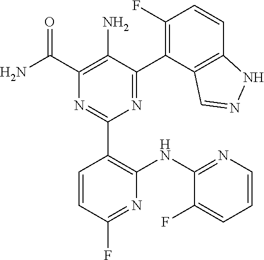 [SMILES: NC(=O)c1nc(-c2ccc(F)nc2Nc2ncccc2F)nc(-c2c(F)ccc3[nH]ncc23)c1N] | 971 |
| 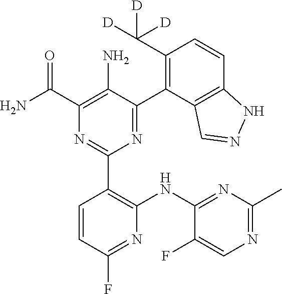 [SMILES: [2H]C([2H])([2H])c1ccc2[nH]ncc2c1-c1nc(-c2ccc(F)nc2Nc2nc(C)ncc2F)nc(C(N)=O)c1N] | 972 |
| 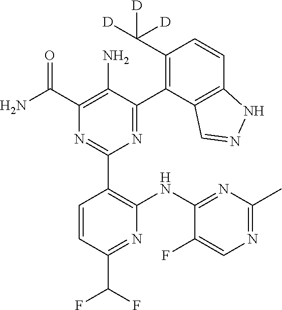 [SMILES: [2H]C([2H])([2H])c1ccc2[nH]ncc2c1-c1nc(-c2ccc(C(F)F)nc2Nc2nc(C)ncc2F)nc(C(N)=O)c1N] | 973 |
| 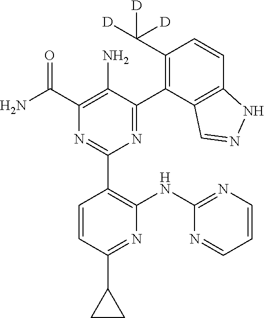 [SMILES: [2H]C([2H])([2H])c1ccc2[nH]ncc2c1-c1nc(-c2ccc(C3CC3)nc2Nc2ncccn2)nc(C(N)=O)c1N] | 974 |
| 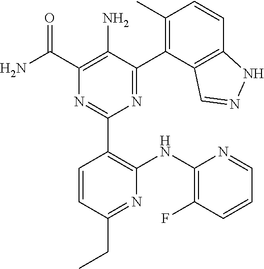 [SMILES: CCc1ccc(-c2nc(C(N)=O)c(N)c(-c3c(C)ccc4[nH]ncc34)n2)c(Nc2ncccc2F)n1] | 975 |
| 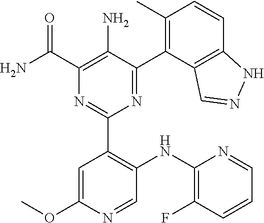 [SMILES: COc1cc(-c2nc(C(N)=O)c(N)c(-c3c(C)ccc4[nH]ncc34)n2)c(Nc2ncccc2F)cn1] | 976 |
| 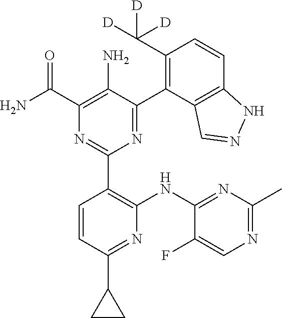 [SMILES: [2H]C([2H])([2H])c1ccc2[nH]ncc2c1-c1nc(-c2ccc(C3CC3)nc2Nc2nc(C)ncc2F)nc(C(N)=O)c1N] | 977 |

The invention includes (where possible) individual diastereomers, enantiomers, epimers, and atropisomers of the compounds disclosed herein, and mixtures of diastereomers and/or enantiomers thereof including racemic mixtures. Although the specific stereochemistries disclosed herein are preferred, other stereoisomers, including diastereomers, enantiomers, epimers, atropisomers, and mixtures of these may also have utility in treating Myt1-mediated diseases. Inactive or less active diastereoisomers and enantiomers may be useful, e.g., for scientific studies relating to the receptor and the mechanism of activation.

It is understood that certain molecules can exist in multiple tautomeric forms. This invention includes all tautomers even though only one tautomer may be indicated in the examples.

The invention also includes pharmaceutically acceptable salts of the compounds, and pharmaceutical compositions comprising the compounds and a pharmaceutically acceptable carrier. The compounds are especially useful, e.g., in certain kinds of cancer and for slowing the progression of cancer once it has developed in a patient.

The compounds disclosed herein may be used in pharmaceutical compositions comprising (a) the compound(s) or pharmaceutically acceptable salts thereof, and (b) a pharmaceutically acceptable carrier. The compounds may be used in pharmaceutical compositions that include one or more other active pharmaceutical ingredients. The compounds may also be used in pharmaceutical compositions in which the compound disclosed herein or a pharmaceutically acceptable salt thereof is the only active ingredient.

### Optical Isomers—Diastereomers—Geometric Isomers—Tautomers

Compounds disclosed herein may contain, e.g., one or more stereogenic centers and can occur as racemates, racemic mixtures, single enantiomers, individual diastereomers, and mixtures of diastereomers and/or enantiomers. The invention includes all such isomeric forms of the compounds disclosed herein. It is intended that all possible stereoisomers (e.g., enantiomers and/or diastereomers) in mixtures and as pure or partially purified compounds are included within the scope of this invention (i.e., all possible combinations of the stereogenic centers as pure compounds or in mixtures).

Some of the compounds described herein may contain bonds with hindered rotation such that two separate rotomers, or atropisomers, may be separated and found to have different biological activity which may be advantageous. It is intended that all of the possible atropisomers are included within the scope of this invention.

Some of the compounds described herein may contain olefinic double bonds, and unless specified otherwise, are meant to include both E and Z geometric isomers.

Some of the compounds described herein may exist with different points of attachment of hydrogen, referred to as tautomers. An example is a ketone and its enol form, known as keto-enol tautomers. The individual tautomers as well as mixtures thereof are encompassed by the invention.

Compounds disclosed herein having one or more asymmetric centers may be separated into diastereoisomers, enantiomers, and the like by methods well known in the art.

Alternatively, enantiomers and other compounds with chiral centers may be synthesized by stereospecific synthesis using optically pure starting materials and/or reagents of known configuration.

### Metabolites—Prodrugs

The invention includes therapeutically active metabolites, where the metabolites themselves fall within the scope of the claims. The invention also includes prodrugs, which are compounds that are converted to the claimed compounds as they are being administered to a patient or after they have been administered to a patient. The claimed chemical structures of this application in some cases may themselves be prodrugs.

### Isotopically Enriched Derivatives

The invention includes molecules which have been isotopically enriched at one or more position within the molecule. Thus, compounds enriched for deuterium fall within the scope of the claims.

### Methods of Preparing a Compound of the Invention

Compounds of the present invention may be prepared using reactions and techniques known in the art and those described herein. One of skill in the art will appreciate that methods of preparing compounds of the invention described herein are non-limiting and that steps within the methods may be interchangeable without affecting the structure of the end product.

### Method A

Compounds of the present invention may be prepared as shown in Scheme A and described herein. The known 8-bromoquinolin-7-amine can be converted to 7-aminoquinoline-8-carbonitrile in presence of copper cyanide which can then be converted to Intermediate A by bromination with NBS. Intermediate A can be arylated via metal-mediated step followed by a nitrile hydrolysis to provide compounds of the present invention. Depending on the nature of the Ar group, a protecting group may be required to be in place prior to the cross-coupling step. In the case where the Ar group bear a protecting group, a deprotection step(s) may be required prior to the nitrile hydrolysis using acid, base and/or fluoride to give compounds of the present invention.

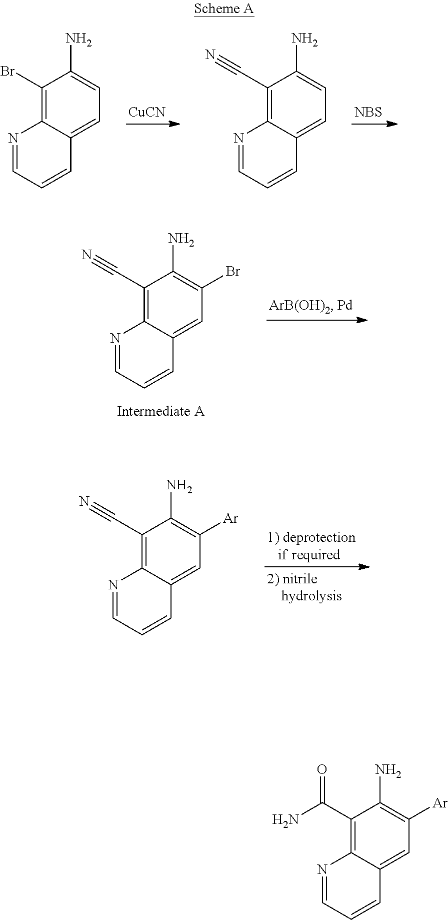

### Method B

Compounds of the present invention may be prepared as shown in Scheme B and described herein. The commercially available ethyl 5-amino-2,6-dichloropyrimidine-4-carboxylate and (5-(methoxymethoxy)-2-methylphenyl)boronic acid can be cross-coupled using a metal-mediated step to provide Intermediate B which can be converted to Intermediate C via a two-step sequence of hydrolysis and amide formation or via a one-step transamidation with ammonia. Intermediate C can be arylated using a metal-mediated cross-coupling step and subsequent deprotection using acidic conditions gives compounds of the present invention.

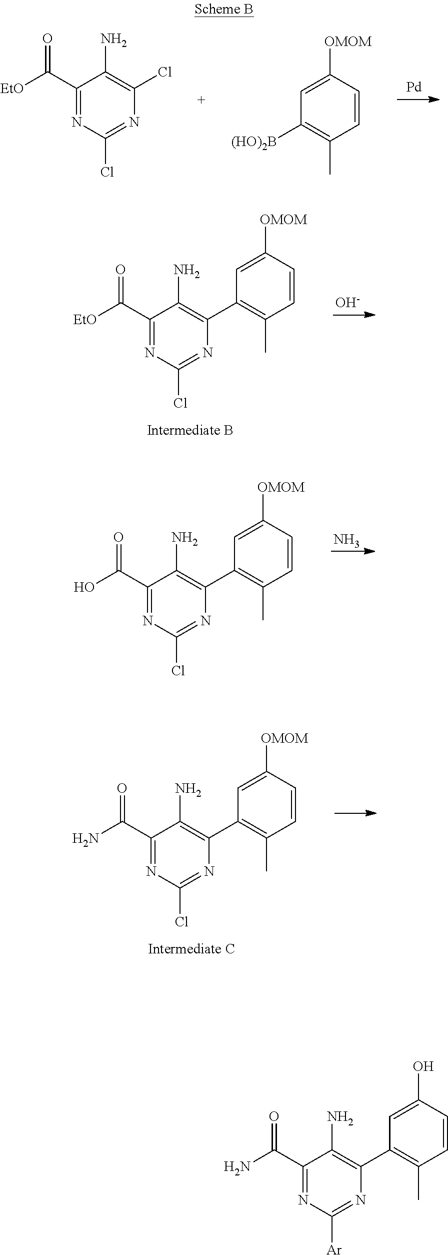

### Method C

Compounds of the present invention may be prepared as shown in Scheme C and described herein. The commercially available 3-amino-6-chloropicolinic acid can be esterified by treatment with a base such as potassium carbonate and ethyl iodide to provide ethyl 3-amino-6-chloropicolinate which can be brominated with NBS to provide Intermediate D. This intermediate can be converted to Intermediate E via a two-step sequence involving a metal-mediated arylation followed by a transamidation reaction using ammonia. Intermediate E can be arylated using a second metal-mediated cross-coupling step and subsequent deprotection with boron tribromide gives compounds of the present invention. Depending on the nature of the aryl group cross-coupled to Intermediate E, an atropisomeric mixture may be obtained. In such cases it may be necessary to isolate the atropisomer of interest to give compounds of the present invention.

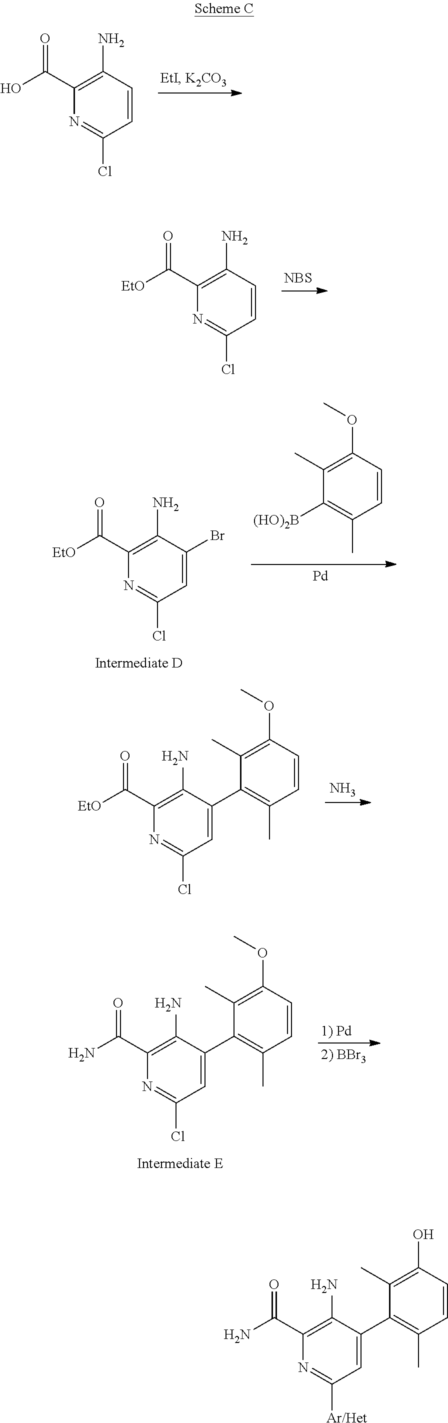

### Method D

Compounds of the present invention may be prepared as shown in Scheme D and described herein. The Intermediate E can be arylated using a metal-mediated cross-coupling step followed by an oxidation of the furan ring to give Intermediate F. This intermediate can be deprotected with boron tribromide and converted to an amide by using an aromatic amine and amide bond forming reagent to provide compounds of the present invention.

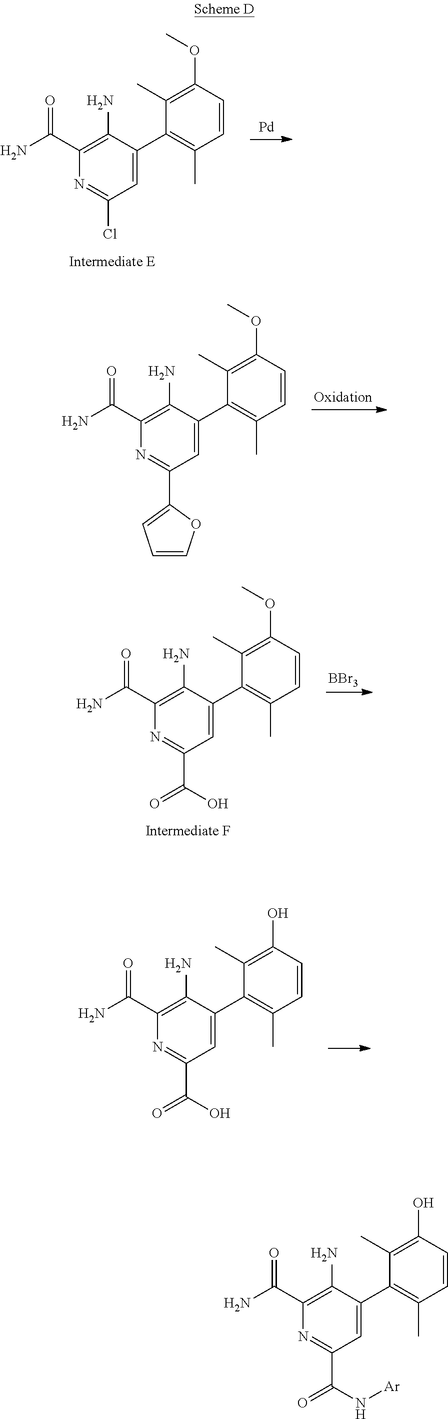

### Method E

Compounds of the present invention may be prepared as shown in Scheme E and described herein. Intermediate D can be arylated with an indazole-4-boronic acid bearing substituents using a metal-mediated cross-coupling step. The resulting ester can be converted to a primary amide upon treatment with ammonia and the arylated using a metal-mediated coupling to give compounds of the present invention. Depending on the nature of the substituents on the indazole group, an atropisomeric mixture may be obtained. In such cases it may be necessary to isolate the atropisomer of interest to give compounds of the present invention.

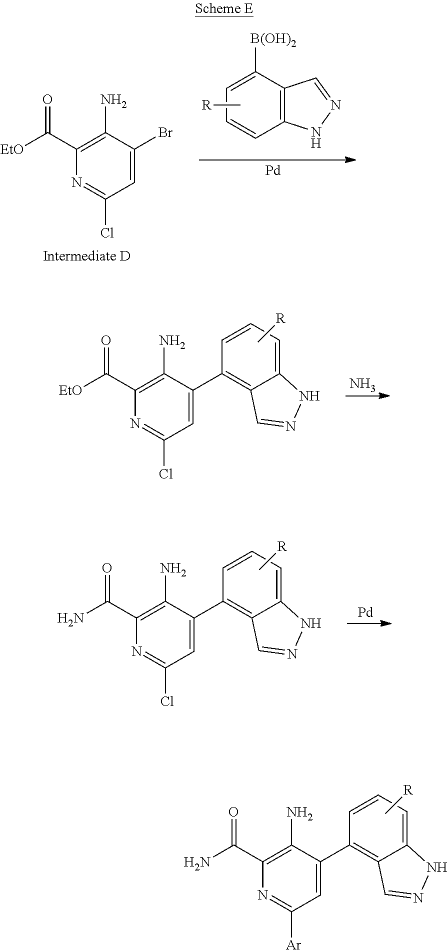

### Method F

Compounds of the present invention may be prepared as shown in Scheme F and described herein. The commercially available kynurenic acid can be esterified under acidic methanolic solution and then converted to methyl 3-chloro kynurenate after chlorination with NCS. This ester can be transformed to a triflate which can then be cross-coupled with a boronic acid via metal-mediated step followed by transamidification to provide compounds of the present invention. In the case where the Ar group bear a protecting group, a deprotection step(s) may be required to give compounds of the present invention.

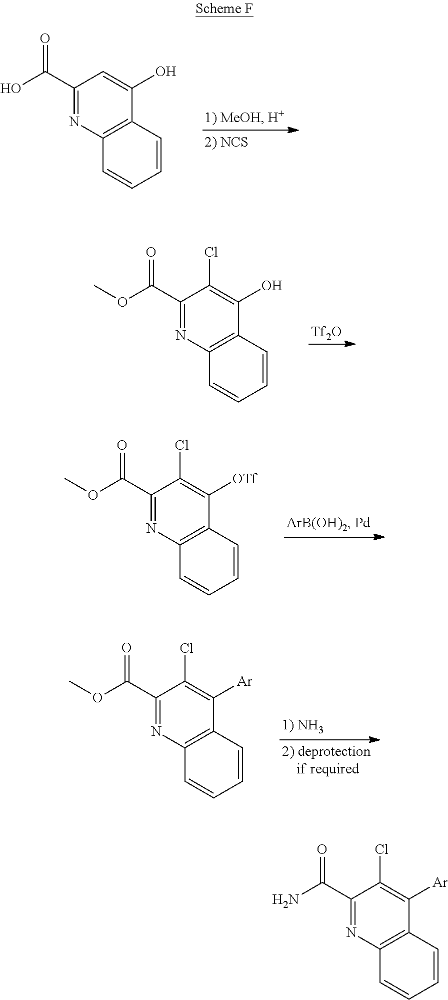

### Method G

Compounds of the present invention may be prepared as shown in Scheme G and described herein. Compound 74 can be reduced under Pd-catalyzed conditions to provide compounds of the present invention.

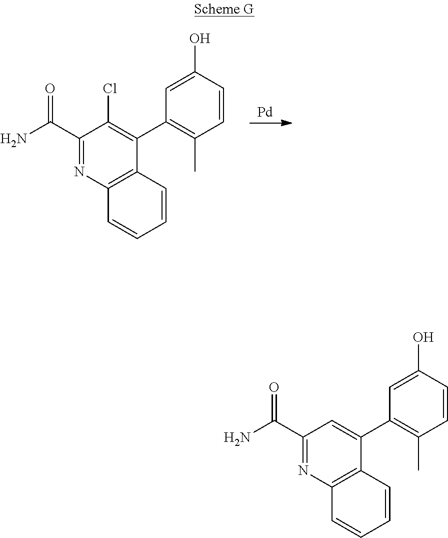

### Method H

Compounds of the present invention may be prepared as shown in Scheme H and described herein. Commercially available methyl 2,5,6-trichloropyrimidine-4-carboxylate can be arylated with an indazole-4-boronic acid bearing substituents using a metal-mediated cross-coupling step. A second arylation can then be performed using a metal-mediated cross-coupling step under which hydrolysis of the ester can occur. Amide formation with ammonia or it's synthetic equivalent can then provide compounds of the present invention.

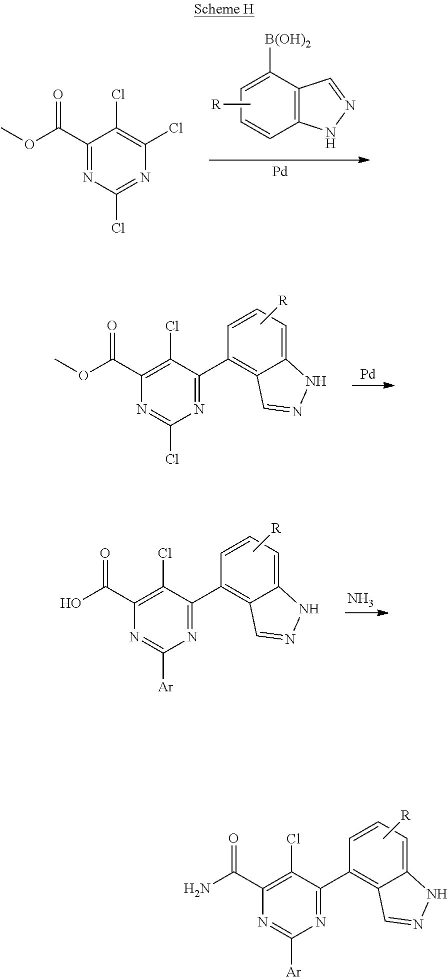

### Method I

Compounds of the present invention may be prepared as shown in Scheme I and described herein. Commercially available methyl 2,6-dichloropyrimidine-4-carboxylate can be arylated with an indazole-4-boronic acid bearing substituents using a metal-mediated cross-coupling step. A second arylation can then be performed using a metal-mediated cross-coupling step under which hydrolysis of the ester can occur. Amide formation with ammonia or it's synthetic equivalent can then provide compounds of the present invention.

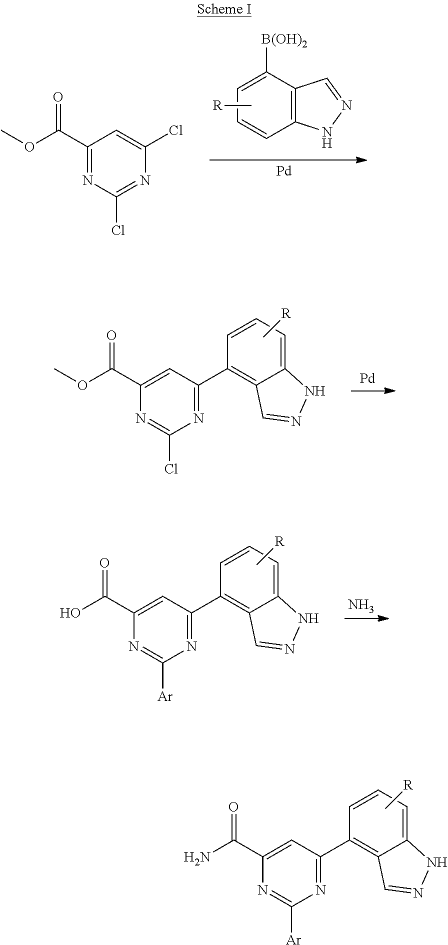

### Method J

Compounds of the present invention may be prepared as shown in Scheme J and described herein. The commercially available 5-amino-2-chloroisonicotinic acid can be esterified by treatment with a base such as potassium carbonate and ethyl iodide to provide ethyl 5-amino-2-chloroisonicotinate which can be brominated with NBS to provide Intermediate G. This intermediate can be converted to Intermediate H via a two-step sequence involving a metal-mediated coupling followed by a transamidation reaction using ammonia. Intermediate H can be arylated using a metal-mediated cross-coupling step and subsequent deprotection with boron tribromide gives compounds of the present invention.

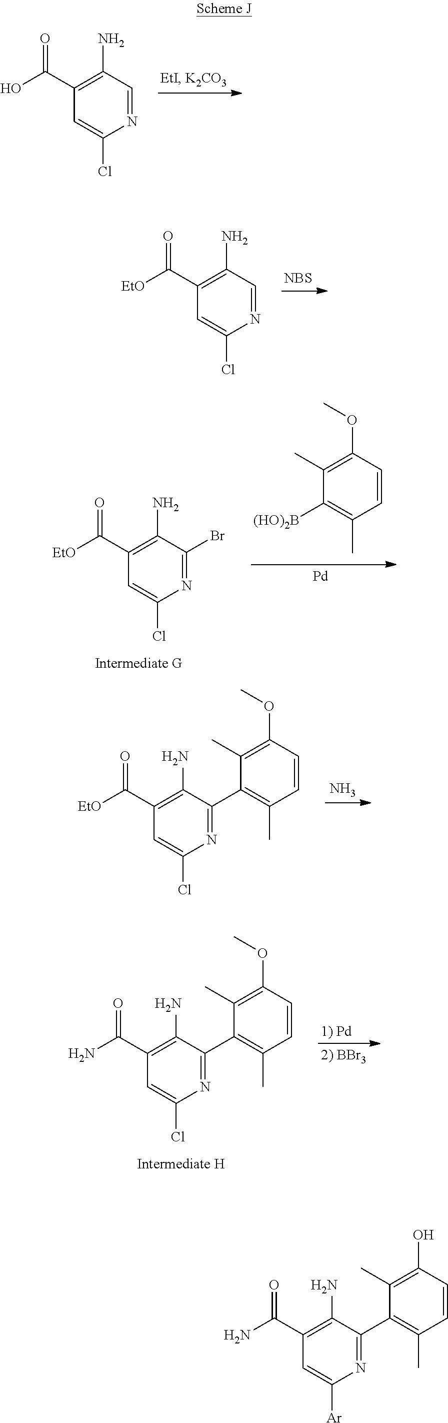

### Method K

Compounds of the present invention may be prepared as shown in Scheme K and described herein. The known 2-amino-5-bromo-3-iodobenzamide can be arylated using a metal-mediated cross-coupling step and then borylated using a second metal-mediated cross-coupling to provide Intermediate I. This intermediate can be arylated using a metal-mediated cross-coupling step and subsequent deprotection with zinc bromide and 1-propanethiol gives compounds of the present invention for which atropisomeric mixture may be obtained. In such cases it may be necessary to isolate the atropisomer of interest to give compounds of the present invention.

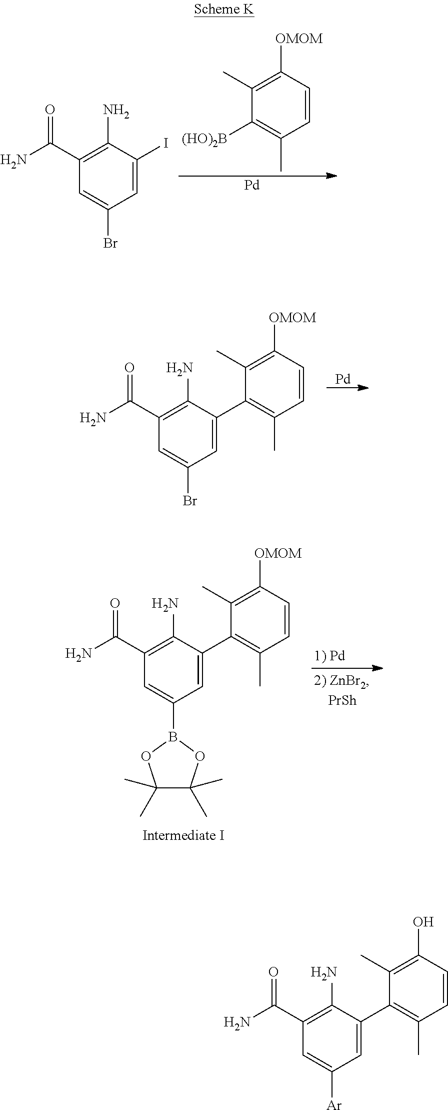

### Method L

Compounds of the present invention may be prepared as shown in Scheme L and described herein. Commercially available ethyl 5-amino-2,6-dichloropyrimidine-4-carboxylate can be arylated with an indazole-4-boronic acid bearing substituents using a metal-mediated cross-coupling step. Hydrolysis of the ester with aqueous solution of NaOH followed by an amide formation by using NH₄Cl, HATU and DIPEA provides the primary amide on which a second arylation can then be performed using a metal-mediated cross-coupling step to provide compounds of the present invention. In the case where the aryl group bear a protecting group, a deprotection step may be required using acid, base and/or fluoride to give compounds of the present invention. In the case where the aryl group bear a racemic appendage, a chiral separation step by SFC may be required to give compounds of the present invention.

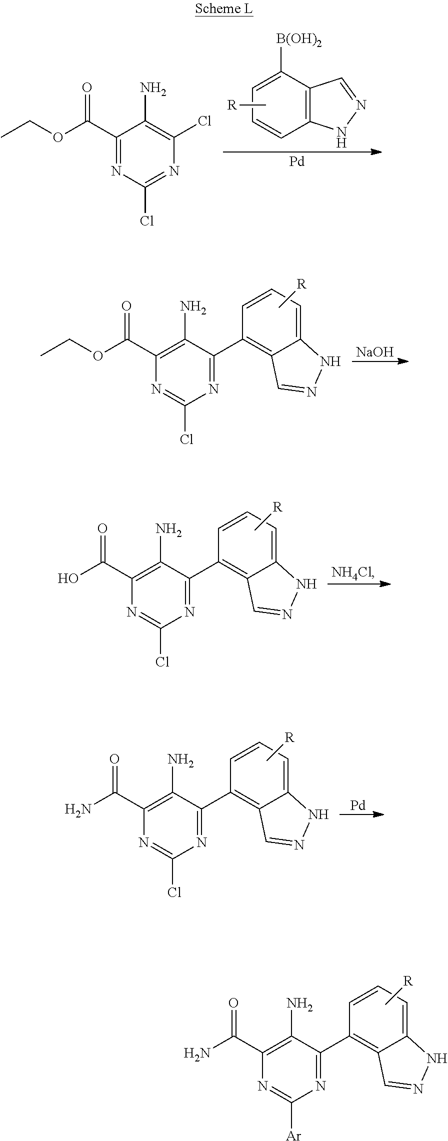

### Method M

Compounds of the present invention may be prepared as shown in Scheme M and described herein. Commercially available ethyl 5-amino-2-chloropyrimidine-4-carboxylate can be arylated with phenylboronic using a metal-mediated cross-coupling step and subsequently brominated using NBS to provide intermediate L. Arylation of this intermediate with a N-THP-protected indazole-4-boronic acid pinacol ester bearing substituents followed by an ammonia mediated transamidation and acidic deprotection of the indazole can then provide compounds of the present invention.

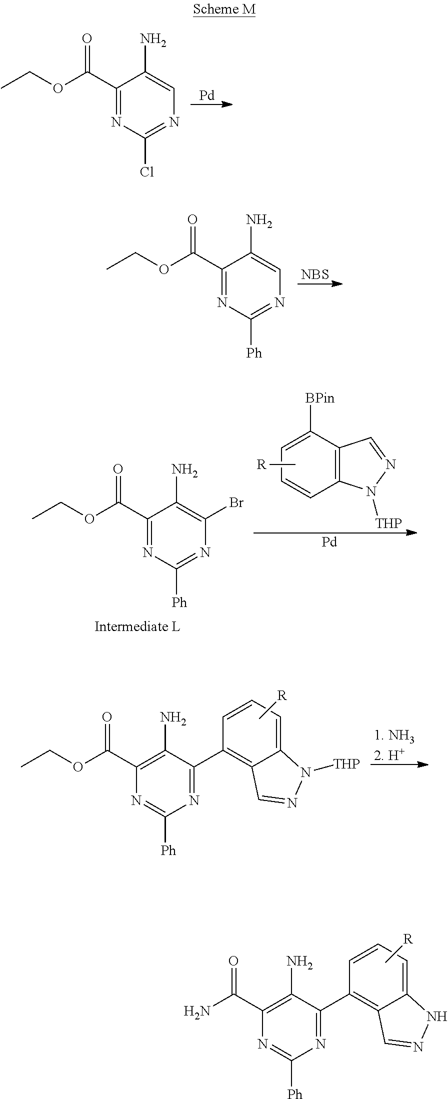

### Method N

Compounds of the present invention may be prepared as shown in Scheme N and described herein. Intermediate G can be arylated with an indazole-4-boronic acid bearing substituents using a metal-mediated cross-coupling step. The resulting ester can be converted to a primary amide upon treatment with ammonia and the arylated using a second metal-mediated cross-coupling to give compounds of the present invention.

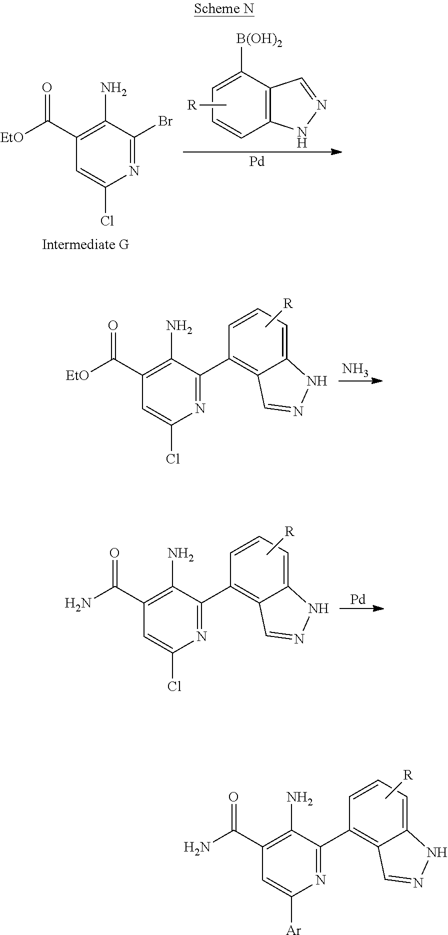

### Method O

Compounds of the present invention may be prepared as shown in Scheme O and described herein. The known 2-amino-5-bromo-3-iodobenzamide can be arylated with (5-methyl-1H-indazol-4-yl)boronic acid using a metal-mediated cross-coupling step and then a second metal-mediated cross-coupling can be performed to provide compounds of the present invention for which atropisomeric mixture may be obtained. In such cases it may be necessary to isolate the atropisomer of interest to give compounds of the present invention.

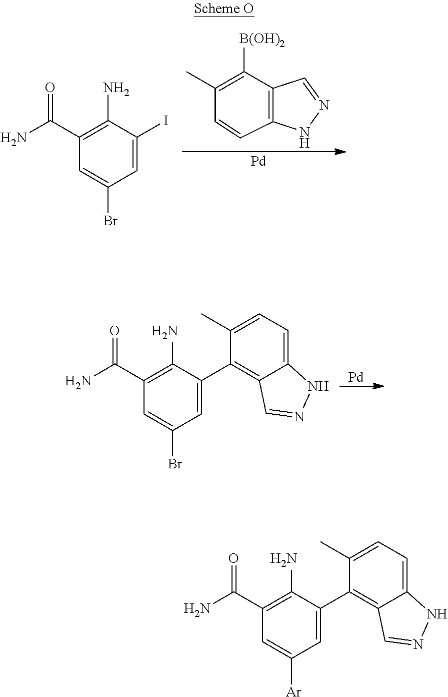

### Method P

Compounds of the present invention may be prepared as shown in Scheme P and described herein. Intermediate K can be arylated with 2-(methylthio)-4-(tributylstannyl)pyrimidine using a metal-mediated cross-coupling step. The resulting methyl thio ether can be oxidized to the methyl sulfone using an oxidizer such as Oxone to provide Intermediate N. This intermediate can undergo a SNAr reaction with a variety of alcohols which may bear other protected moiety to give compounds of the present invention after deprotection and for which atropisomeric mixture may be obtained. In such cases it may be necessary to isolate the atropisomer of interest to give compounds of the present invention.

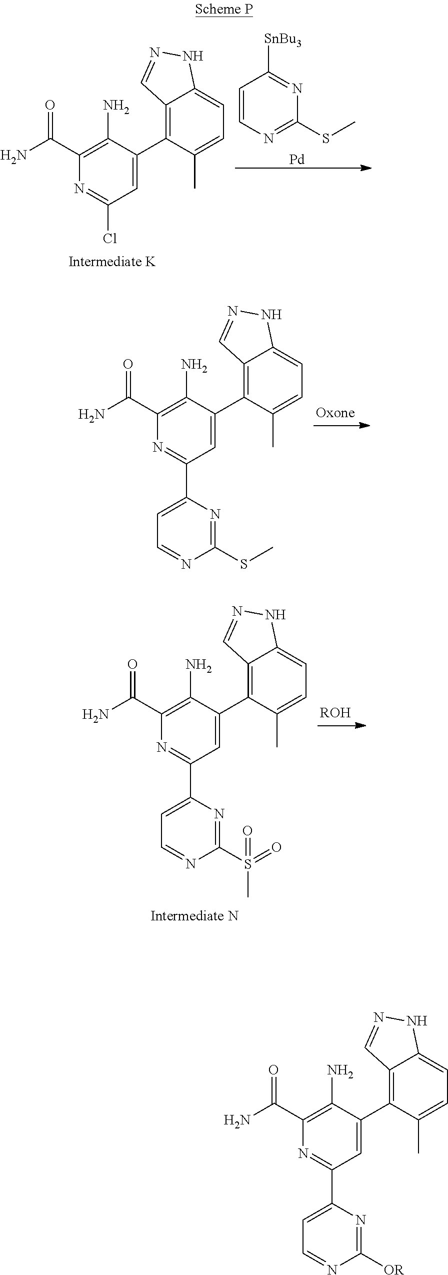

### Method Q

Compounds of the present invention may be prepared as shown in Scheme Q and described herein. Intermediate N can undergo a SNAr reaction with a variety of amines to give compounds of the present invention for which atropisomeric mixture may be obtained. In such cases it may be necessary to isolate the atropisomer of interest to give compounds of the present invention.

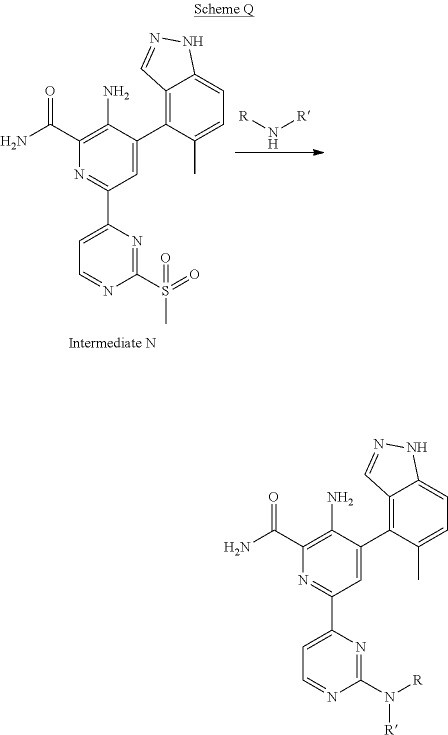

### Method R

Compounds of the present invention may be prepared as shown in Scheme R and described herein. Intermediate K can be arylated with 2-fluoro-6-(tributylstannyl)pyridine using a metal-mediated cross-coupling step to provide Intermediate O. This intermediate can undergo a SNAr reaction with a variety of alcohols to give compounds of the present invention for which atropisomeric mixture may be obtained. In such cases it may be necessary to isolate the atropisomer of interest to give compounds of the present invention.

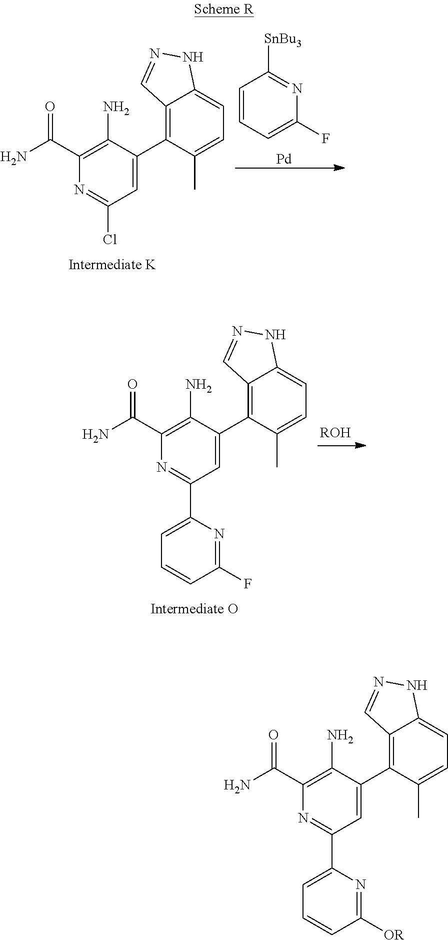

### Method S

Compounds of the present invention may be prepared as shown in Scheme S and described herein. Intermediate K can be arylated with tributyl(aryl)stannanes using a metal-mediated cross-coupling step under which n-butylated products can be isolated as side-products to give compounds of the present invention.

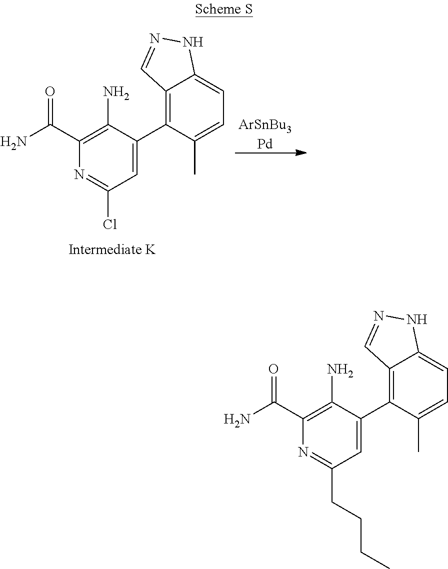

### Method T

Compounds of the present invention may be prepared as shown in Scheme T and described herein. Substituted 4-bromo- or 4-chloro-1-(THP)-1H-indazole can be borylated using a metal-mediated cross-coupling step to provide pinacol (THP)-1H-indazol-4-yl)boronates. These boronates can undergo a metal-mediated cross-coupling step with Intermediate P to provide THP-protected esters that can be transamidified with ammonia and deprotected under acidic conditions to give compounds of the present invention for which atropisomeric mixture may be obtained. In such cases it may be necessary to isolate the atropisomer of interest to give compounds of the present invention.

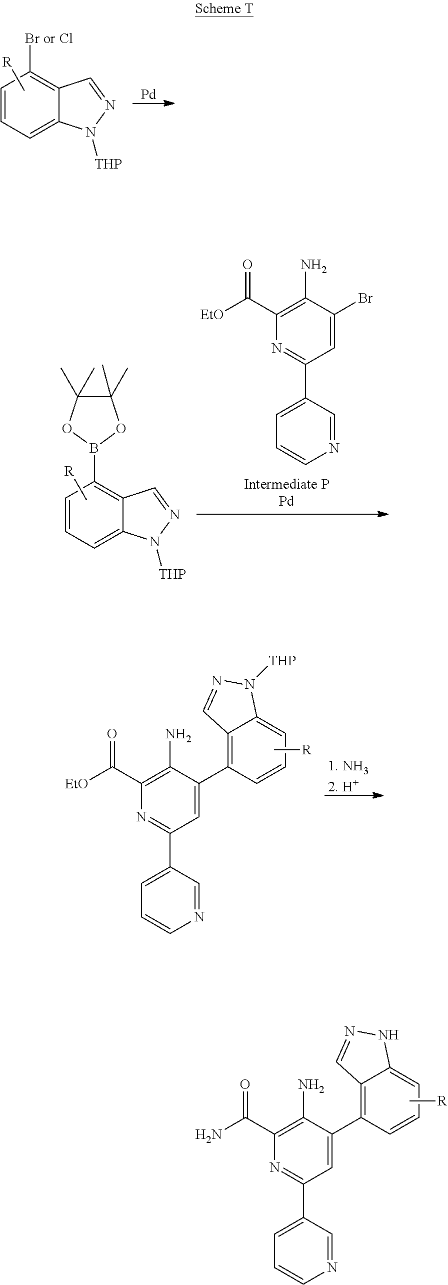

### Method U

Compounds of the present invention may be prepared as shown in Scheme U and described herein. Commercially available 3-amino-5-methylpicolinic acid hydrochloride can be converted to a primary amide by using HATU and an ammonia surrogate. This primary amide can then be brominated to provide 3-amino-4,6-dibromo-5-methylpicolinamide. This compound can be diarylated using metal-mediated cross-coupling steps using 2-(tributylstannyl)pyridine and pinacol (1H-indazol-4-yl)boronic ester respectively to give compounds of the present invention.

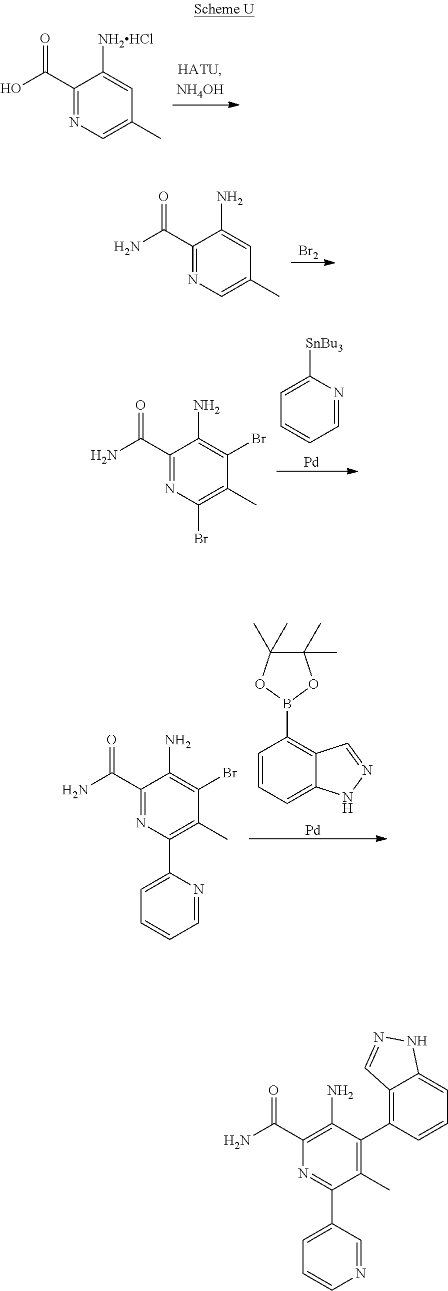

### Method V

Compounds of the present invention may be prepared as shown in Scheme V and described herein. Intermediate 0 can undergo a SNAr reaction with a variety of amines to give compounds of the present invention for which atropisomeric mixture may be obtained. In such cases it may be necessary to isolate the atropisomer of interest to give compounds of the present invention.

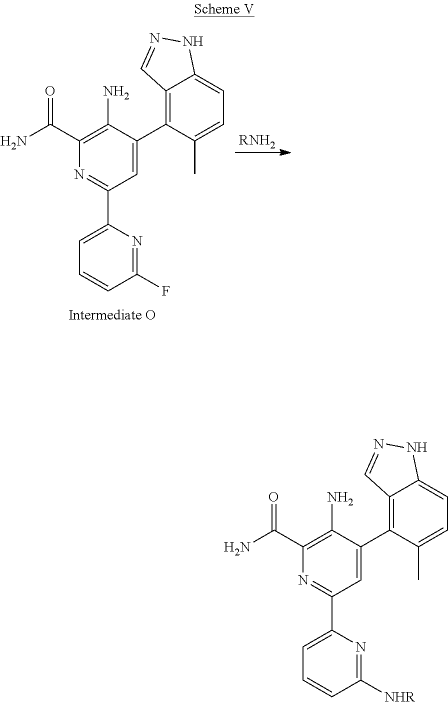

### Method W

Compounds of the present invention may be prepared as shown in Scheme W and described herein. Intermediate J can be arylated with (3-(((tert-butoxycarbonyl)amino)methyl)phenyl)boronic acid using a metal-mediated cross-coupling step. Deprotection under acidic conditions followed by SNAr reaction with substituted 2-halopyrimidines can then provide compounds of the present invention.

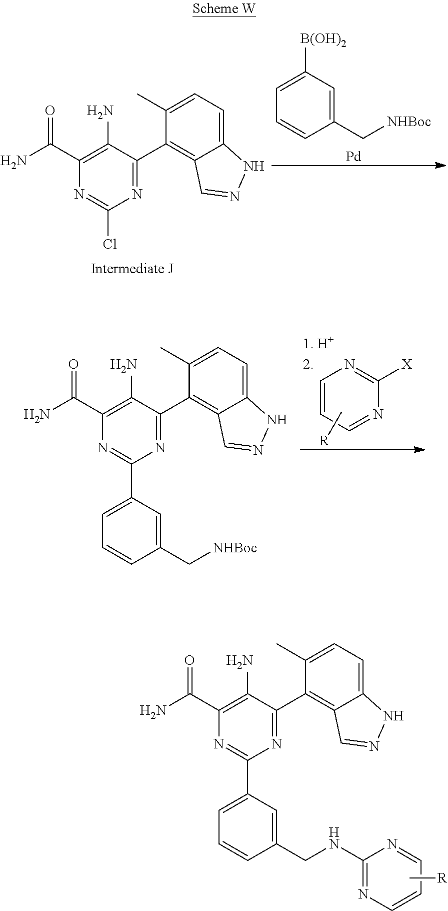

### Method X

Compounds of the present invention may be prepared as shown in Scheme X and described herein. Intermediate K can be protected with di-tert-butyl dicarbonate and the be aminated with anilines using a metal-mediated cross-coupling step to provide compounds of the present invention invention for which atropisomeric mixture may be obtained. In such cases it may be necessary to isolate the atropisomer of interest to give compounds of the present invention.

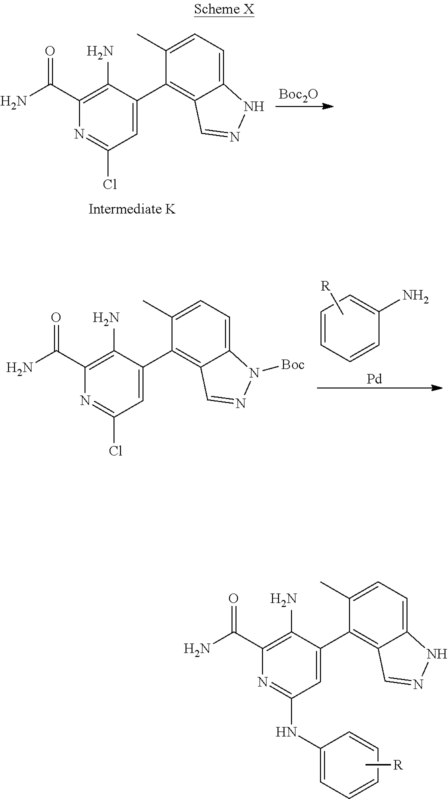

### Method Y

Intermediates used to make compounds of the present invention may be prepared as shown in Scheme Y and described herein. Commercially available 2-(4,4,5,5-tetramethyl-1,3,2-dioxaborolan-2-yl)anilines which can bear substitutions can be acylated using acid chlorides or by using carboxylic acids and amide formation reagents such as HATU to obtain pinacol boronate esters that can be used with methods described herein to provide compounds of the present invention invention.

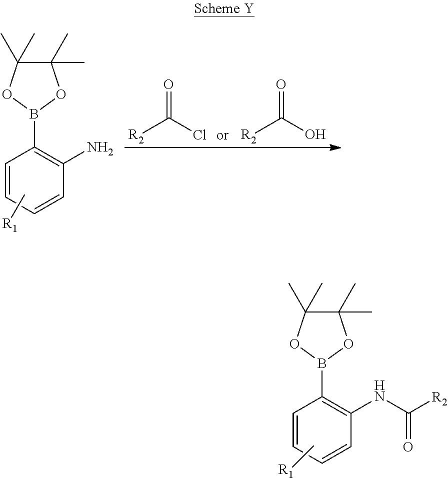

### Method Z

Intermediates used to make compounds of the present invention may be prepared as shown in Scheme Z and described herein. Commercially available 2-bromoanilines which can bear substitutions can be alkylated using alkyl halides or pseudo-halides. These alkylated anilines can then be borylated using a metal-mediated cross-coupling step to obtain pinacol boronate esters that can be used with methods described herein to provide compounds of the present invention invention.

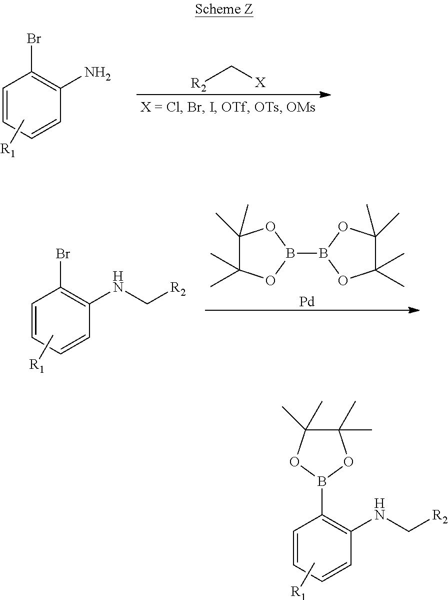

### Method AA

Intermediates used to make compounds of the present invention may be prepared as shown in Scheme AA and described herein. Commercially available 2-bromoanilines which can bear substitutions can be alkylated using aldehydes or ketones in reductive amination reaction. These alkylated anilines can then be borylated using a metal-mediated cross-coupling step to obtain pinacol boronate esters that can be used with methods described herein to provide compounds of the present invention invention.

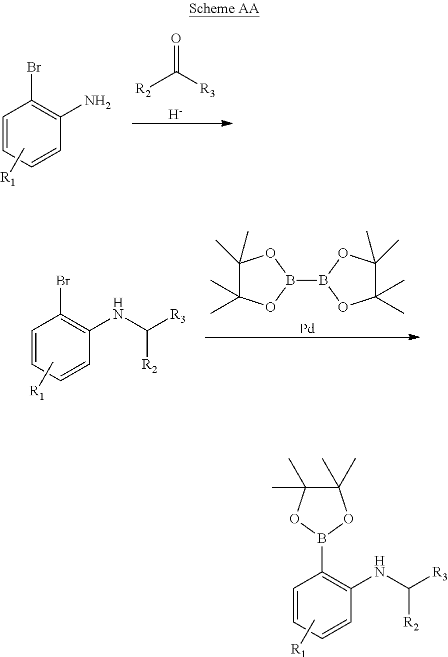

### Method AB

Compounds of the present invention may be prepared as shown in Scheme AB and described herein. Commercially available 5-bromo-6-fluoroindoline-2,3-dione can be arylated with (2-hydroxyphenyl)boronic acid using a metal-mediated cross-coupling step and then cyclized using a base like potassium tert-butoxide to provide 1H-benzofuro[3,2-f]indole-2,3-dione. This compound can then be hydrolyzed and oxidized in a one pot procedure with NaOH and H₂O₂ and subsequently transformed into a primary amide by using standard amide coupling reagents such as HATU and ammonium chloride. This carboxamide can then be brominated with a brominating reagent such as NBS and subsequently arylated with (5-methyl-1H-indazol-4-yl)boronic acid using a metal-mediated cross-coupling step to provide compounds of the present invention.

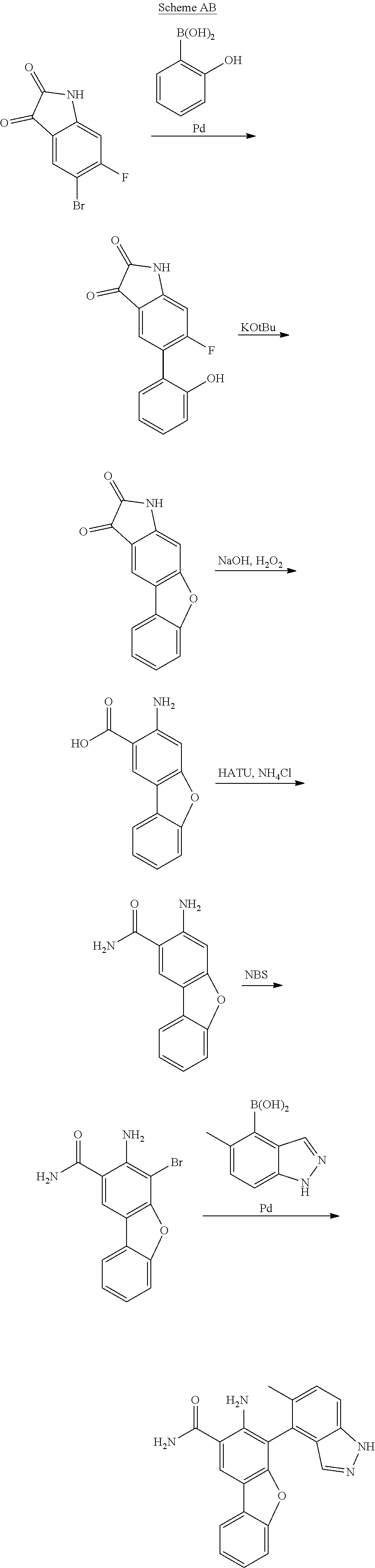

### Method AC

Compounds of the present invention may be prepared as shown in Scheme AC and described herein. Intermediate J can be arylated with 2-(4,4,5,5-tetramethyl-1,3,2-dioxaborolan-2-yl)anilines which can bear substitutions using a metal-mediated cross-coupling step and subsequently sulfonylated using sulfonyl chlorides to provide compounds of the present invention.

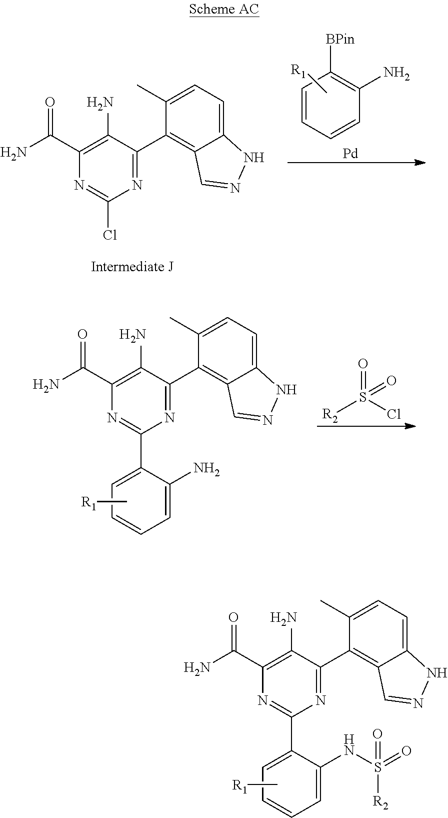

### Method AD

Compounds of the present invention may be prepared as shown in Scheme AD and described herein. 5-amino-2-chloro-6-(1H-indazol-4-yl)pyrimidine-4-carboxamide bearing substitutions on the indazole moiety can be arylated with borylated or stannylated substituted anilines using a metal-mediated cross-coupling step and subsequently N-arylated under acidic SNAr conditions to provide compounds of the present invention.

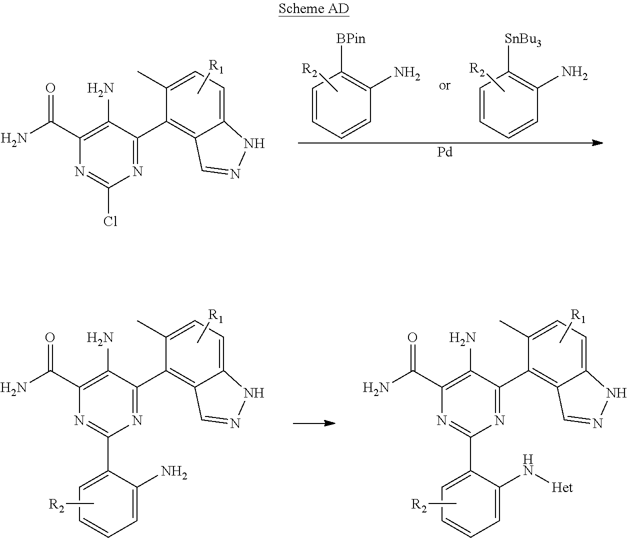

### Method AE

Intermediates used to make compounds of the present invention may be prepared as shown in Scheme AE and described herein. Commercially available 2-(4,4,5,5-tetramethyl-1,3,2-dioxaborolan-2-yl)anilines which can bear substitutions can be acylated using acid chlorides or by using carboxylic acids and amide formation reagents such as HATU to obtain amides that can be subsequently reduced using a reducing reagent such as borane to provide pinacol boronate esters used with methods described herein to provide compounds of the present invention invention.

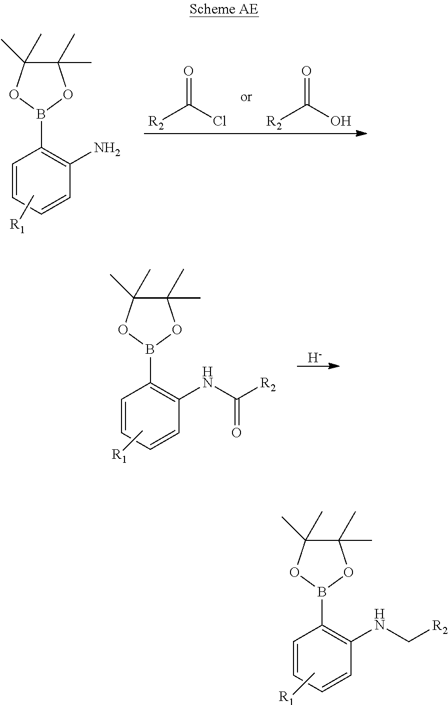

### Method AF

Compounds of the present invention may be prepared as shown in Scheme AF and described herein. Intermediate J can be arylated with 2-(4,4,5,5-tetramethyl-1,3,2-dioxaborolan-2-yl)anilines which can bear substitutions using a metal-mediated cross-coupling step and subsequently acylated using acid chlorides or carboxylic acids and amide formation reagents such as HATU to provide compounds of the present invention.

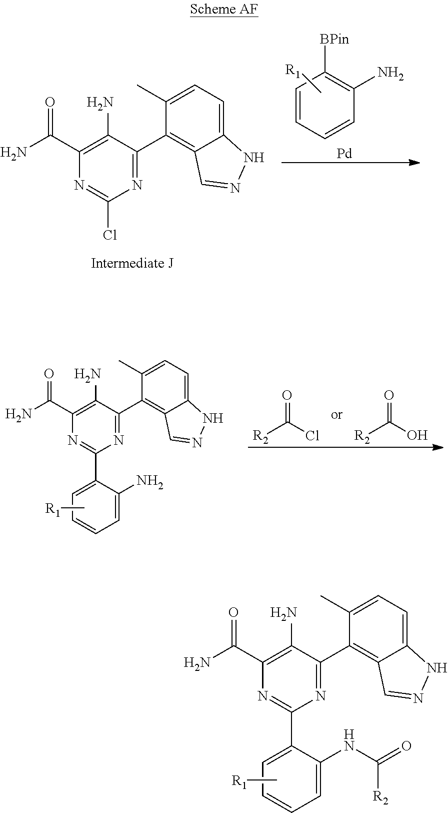

### Method AG

Compounds of the present invention may be prepared as shown in Scheme AG and described herein. Commercially available 6-amino-3-bromo-2-fluorobenzonitrile can be protected with 2,5-hexanedione under acidic conditions and subsequently cross-coupled to Intermediate V under a metal-mediated step to provide the protected 7-aminobenzofuro[3,2-b]pyridine-6-carbonitrile. This intermediate can be hydrolyzed to the the carboxamide under basic conditions, deprotected using hydroxylamine, brominated with a brominating reagent such as NBS and subsequently arylated with arylated with (5-methyl-1H-indazol-4-yl)boronic acid using a metal-mediated cross-coupling step to provide compounds of the present invention.

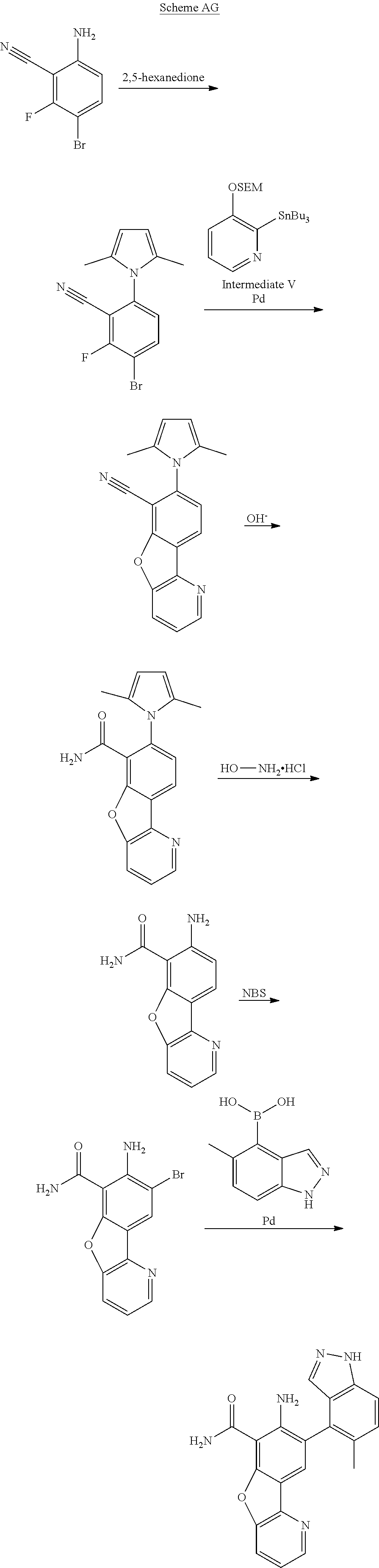

### Method AH

Intermediates used to make compounds of the present invention may be prepared as shown in Scheme AH and described herein. Commercially available 2-(4,4,5,5-tetramethyl-1,3,2-dioxaborolan-2-yl)anilines which can bear substitutions can be reductively aminated with ketones or aldehydes using a reducing agent such as sodium cyanoborohydride to obtain pinacol boronate esters that can be used with methods described herein to provide compounds of the present invention invention.

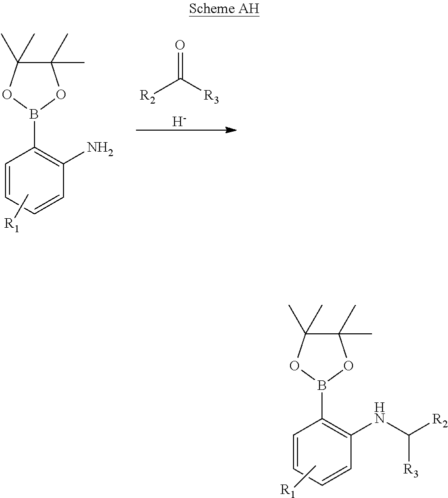

### Method AI

Compounds of the present invention may be prepared as shown in Scheme AI and described herein. Commercially available methyl 2,6-dichloropyrimidine-4-carboxylate can be arylated with a N-THP-protected pinacol indazole-4-boronic ester bearing substituents using a metal-mediated cross-coupling step. A second metal-mediated cross-coupling step can be used to install a 3-(2-amino-pyridyl) group which can also bear substitutents. The primary amine can then be N-arylated using a metal-mediated cross-coupling step. Transamidation with ammonia followed by cleavage of the THP group under acidic conditions can then provide compounds of the present invention.

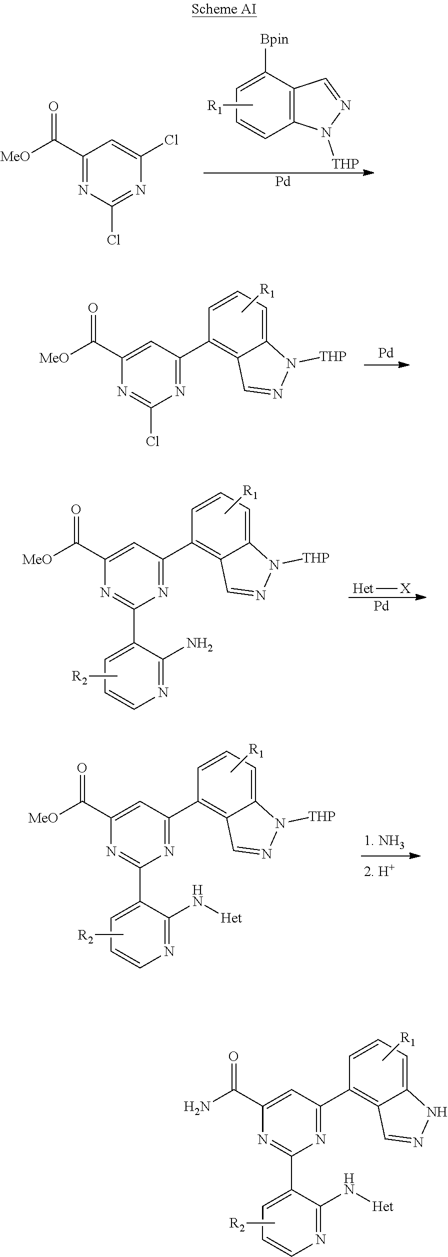

### Method AJ

Compounds of the present invention may be prepared as shown in Scheme AJ and described herein. Commercially available ethyl 2,6-dichloro-5-fluoropyrimidine-4-carboxylate can be arylated with a N-THP-protected pinacol indazole-4-boronic ester bearing substituents using a metal-mediated cross-coupling step. A second metal-mediated cross-coupling step can be used to install a 3-(2-amino-pyridyl) group which can also bear substitutents. The primary amine can then be N-arylated using a metal-mediated cross-coupling step. Transamidation with ammonia followed by cleavage of the THP group under acidic conditions can then provide compounds of the present invention.

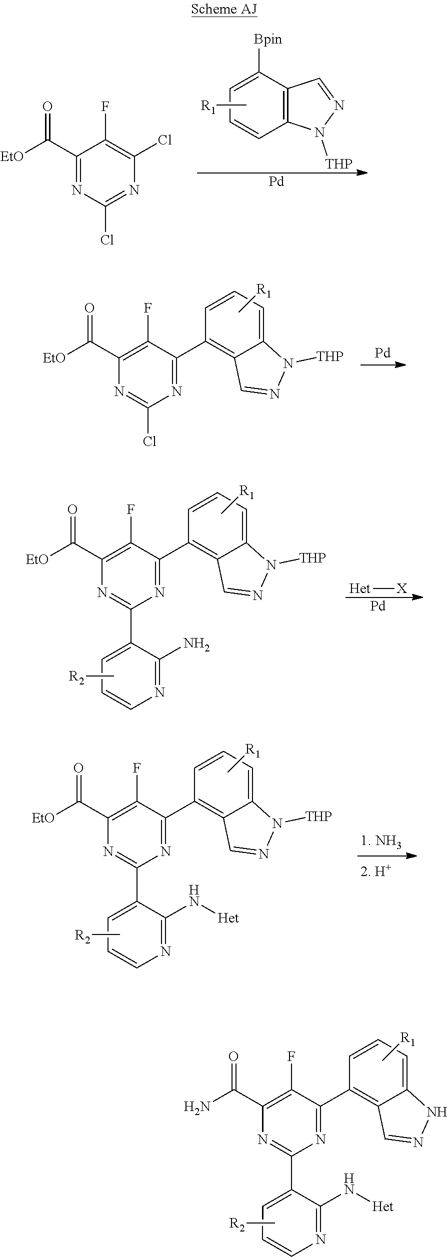

### Method AK

Compounds of the present invention may be prepared as shown in Scheme AK and described herein. Commercially available ethyl 5-amino-2,6-dichloropyrimidine-4-carboxylate can be arylated with a N-THP-protected pinacol indazole-4-boronic ester bearing substituents using a metal-mediated cross-coupling step. A second metal-mediated cross-coupling step can be used to install a 3-(2-amino-pyridyl) group which can also bear substitutents. The primary amine can then be N-arylated using a metal-mediated cross-coupling step. Transamidation with ammonia followed by cleavage of the THP group under acidic conditions can then provide compounds of the present invention.

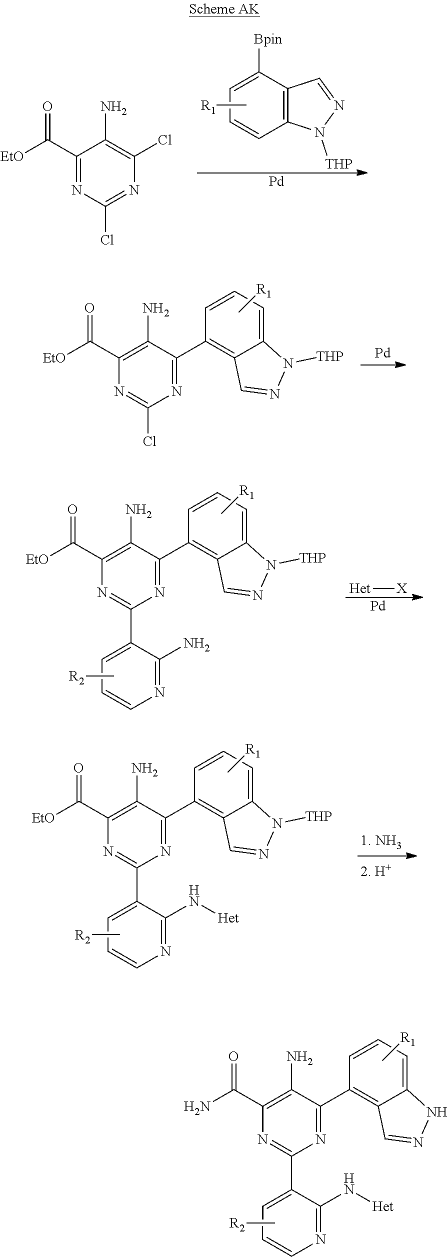

### Method AL

Compounds of the present invention may be prepared as shown in Scheme AL and described herein. Intermediate J can be arylated with an appropriate boronic acid or ester or with an appropriate tributylstannane using a metal-mediated cross-coupling step to install a 3-(2-fluoro-pyridyl) group which can also bear substitutents. These 2-fluoropyridines can then be submitted to SNAr reaction conditions with primary amines to provide compounds of the present invention.

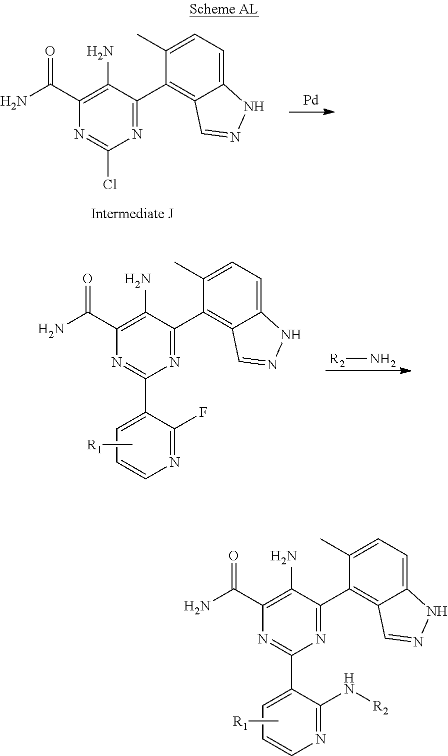

### Method AM

Intermediates used to make compounds of the present invention may be prepared as shown in Scheme AM and described herein. Commercially available 2-bromo-1-iodobenzenes which can bear substitutions can be N-arylated with commercially available arylamines or heteroaryl amines using a metal-mediated cross-coupling step. These secondary anilines can then be borylated using a metal-mediated cross-coupling step to obtain pinacol boronate esters that can be used with methods described herein to provide compounds of the present invention invention.

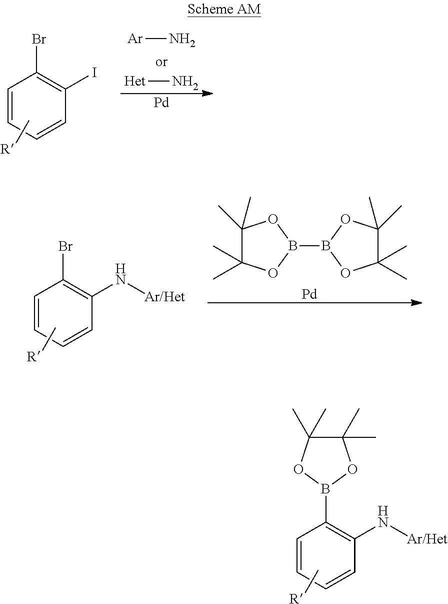

### Method AN

Compounds of the present invention may be prepared as shown in Scheme AN and described herein. Commercially available ethyl 5-amino-2,6-dichloropyrimidine-4-carboxylate can be arylated with a N-THP-protected pinacol indazole-4-boronic ester bearing substituents using a metal-mediated cross-coupling step. A second metal-mediated cross-coupling step can be used to install a 3-(4-fluoro-pyridyl) group which can also bear substitutents. These 2-fluoropyridines can then be submitted to SNAr reaction conditions with primary amines and subsequently be transamidified with ammonia and deprotected under acidic conditions to provide compounds of the present invention.

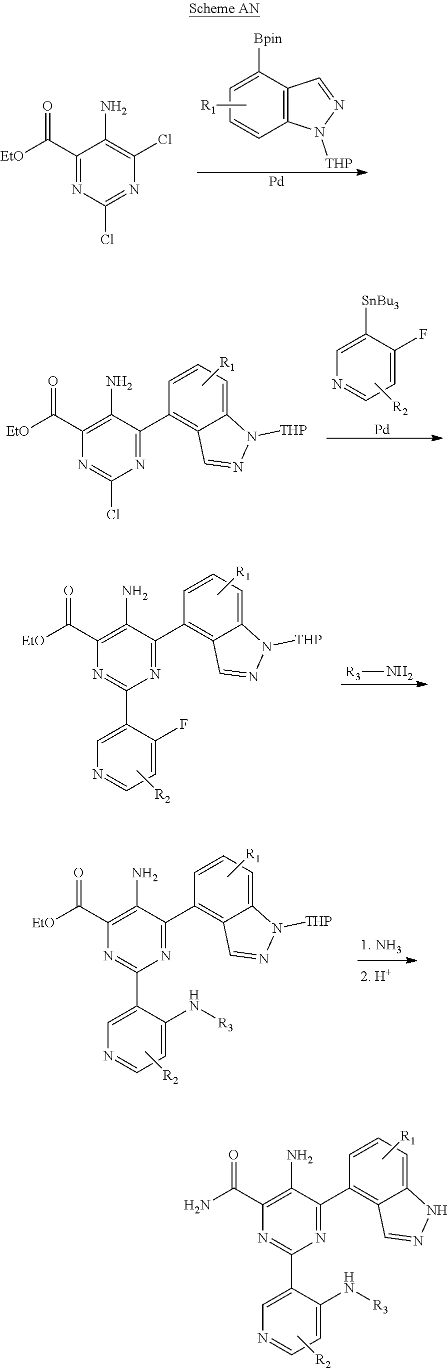

### Method AO

Compounds of the present invention may be prepared as shown in Scheme AO and described herein. Intermediate J can be stannylated with bis(tributyltin) using a metal-mediated cross-coupling step to provide Intermediate X. A second metal-mediated cross-coupling step can be used to install an aryl or heteroaryl group with or without substituents to provide compounds of the present invention.

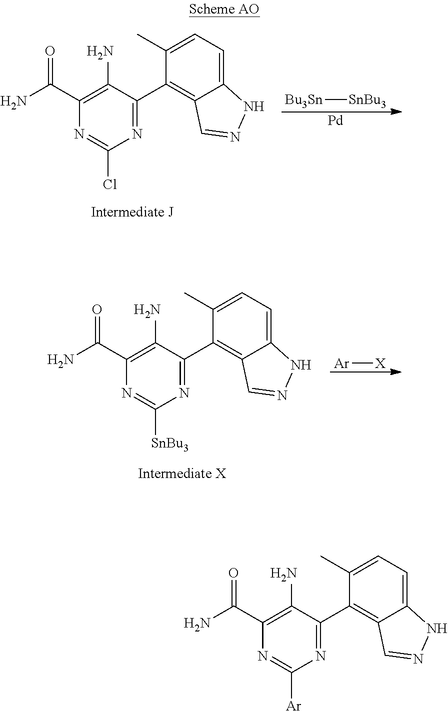

### Method AP

Compounds of the present invention may be prepared as shown in Scheme AP and described herein. Intermediate J can first be borylated with bis(pinacolato)diboron using a metal-mediated cross-coupling step and then arylated using second metal-mediated cross-coupling step to install an aryl or heteroaryl group with or without substituents to provide compounds of the present invention.

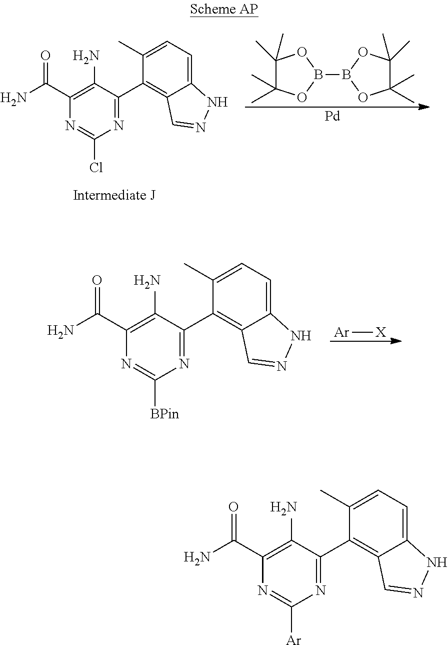

### Method AQ

Compounds of the present invention may be prepared as shown in Scheme AQ and described herein. SNAr reactions can be performed on Intermediate Z using alcohols and a base such as NaH. The resulting intermediate can by hydrolyzed to the primary amide using the Ghaffar-Parkins catalyst to provide compounds of the present invention for which atropisomeric mixture may be obtained. In such cases it may be necessary to isolate the atropisomer of interest to give compounds of the present invention.

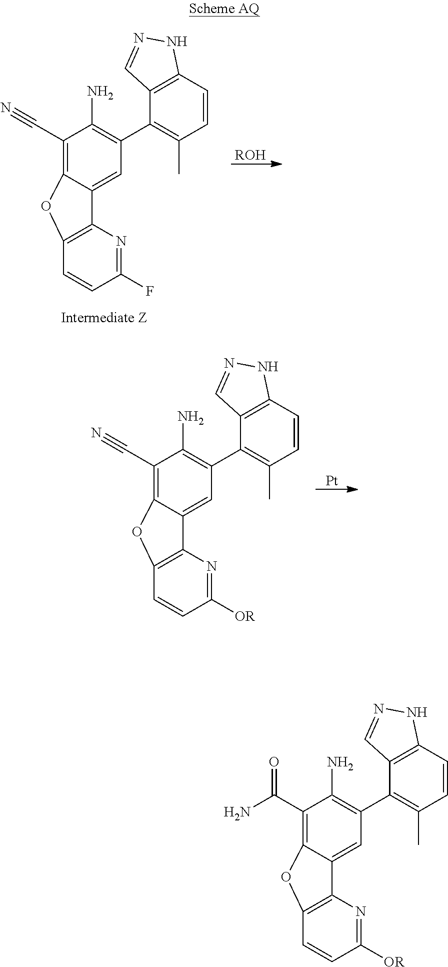

### Method AR

Compounds of the present invention may be prepared as shown in Scheme AR and described herein. SNAr reactions can be performed on Intermediate Z using amines. The resulting intermediate can by hydrolyzed to the primary amide using the Ghaffar-Parkins catalyst to provide compounds of the present invention for which atropisomeric mixture may be obtained. In such cases it may be necessary to isolate the atropisomer of interest to give compounds of the present invention.

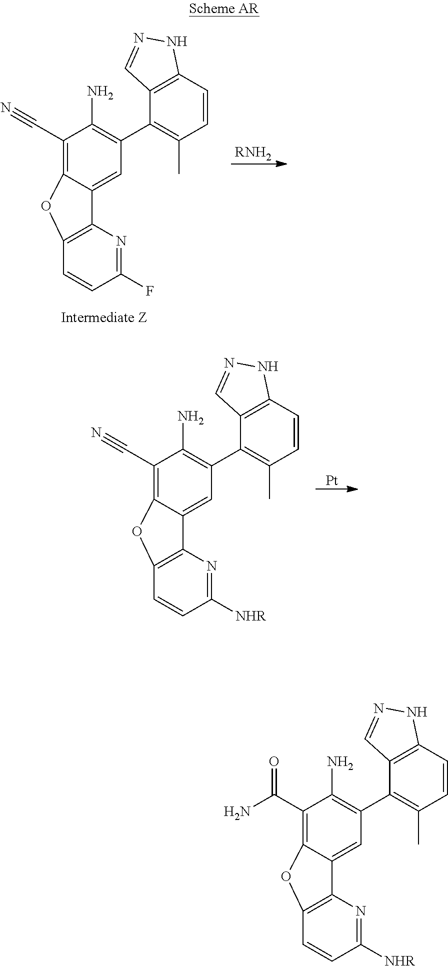

### Method AS

Compounds of the present invention may be prepared as shown in Scheme AS and described herein. Commercially available ethyl 5-amino-2,6-dichloropyrimidine-4-carboxylate can be arylated with a N-THP-protected pinacol indazole-4-boronic ester bearing substituents using a metal-mediated cross-coupling step. A second metal-mediated cross-coupling step can be used to install a 3-(2-amino-phenyl) group which can also bear substitutents. The primary amine can then be N-arylated using a metal-mediated cross-coupling step. Transamidation with ammonia followed by cleavage of the THP group under acidic conditions can then provide compounds of the present invention.

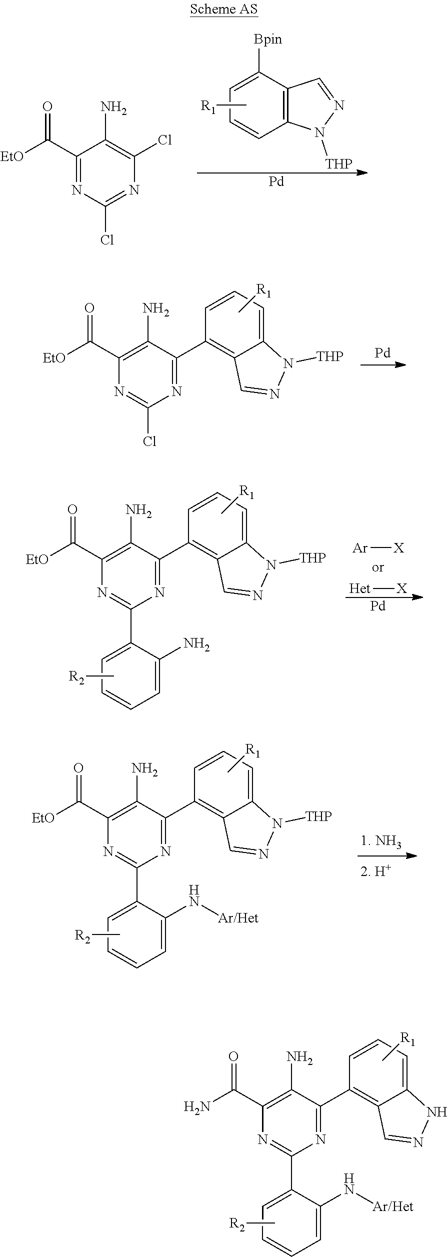

### Method AT

Compounds of the present invention may be prepared as shown in Scheme AT and described herein. Commercially available ethyl 5-amino-2,6-dichloropyrimidine-4-carboxylate can be arylated with a N-THP-protected pinacol indazole-4-boronic ester bearing substituents using a metal-mediated cross-coupling step. This chloropyrimidine can be stannylated with bis(tributyltin) using a metal-mediated cross-coupling step. A third metal-mediated cross-coupling step can be used to cross-couple an aryl or heteroaryl group which can also bear substitutents. Transamidation with ammonia followed by cleavage of the THP group under acidic conditions can then provide compounds of the present invention.

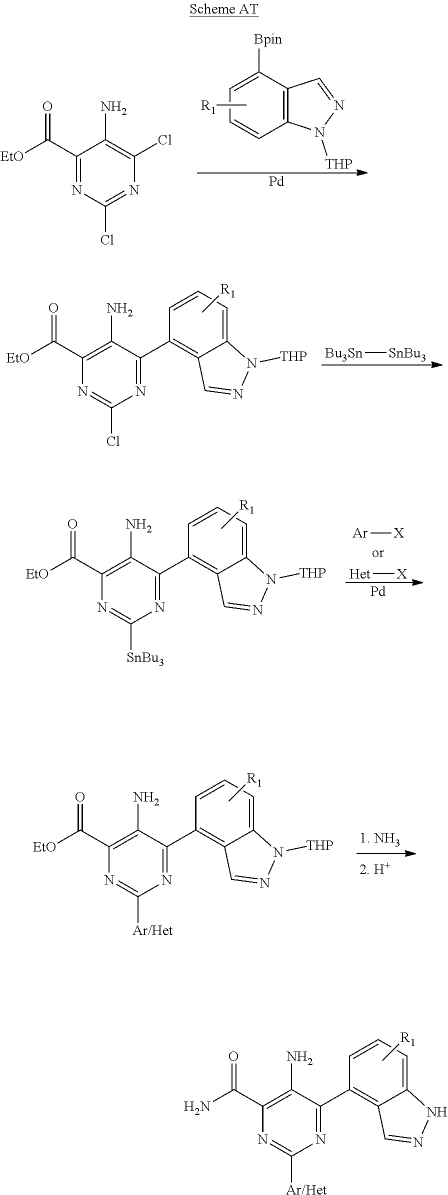

### Method AU

Compounds of the present invention may be prepared as shown in Scheme AU and described herein. Stannylated N-THP protected 5-amino-6-(1H-indazol-4-yl)pyrimidine-4-carboxamides bearing substituents on the indazole moiety can be arylated with 3-bromopyridnes using a metal-mediated cross-coupling step to install a 3-(2-amino-pyridyl) group which can also bear substitutents. The primary amine can then be N-arylated using a metal-mediated cross-coupling step. Transamidation with ammonia followed by cleavage of the THP group under acidic conditions can then provide compounds of the present invention.

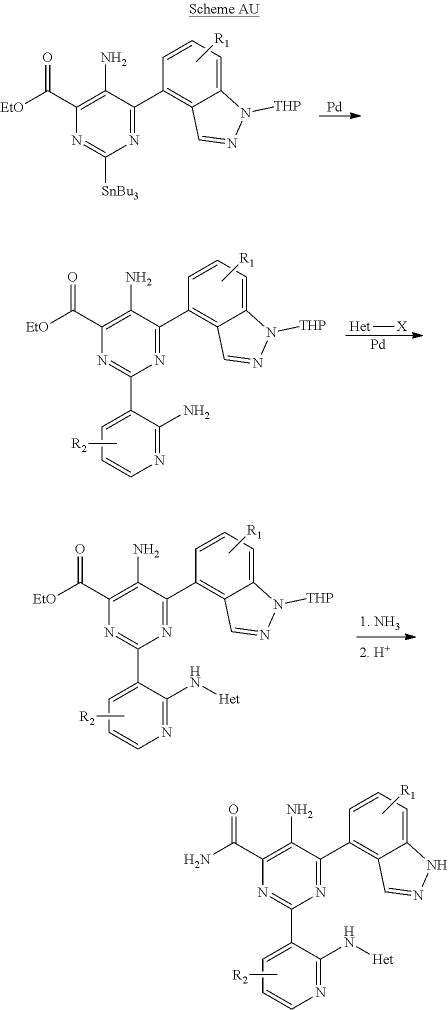

### Method AV

Compounds of the present invention may be prepared as shown in Scheme AV and described herein. Stannylated N-THP protected 5-amino-6-(1H-indazol-4-yl)pyrimidine-4-carboxamides bearing substituents on the indazole moiety can be arylated with 2-bromoanilines using a metal-mediated cross-coupling step to install a 3-(2-aminophenyl) group which can also bear substitutents. The primary amine can then be N-arylated using a metal-mediated cross-coupling step. Transamidation with ammonia followed by cleavage of the THP group under acidic conditions can then provide compounds of the present invention.

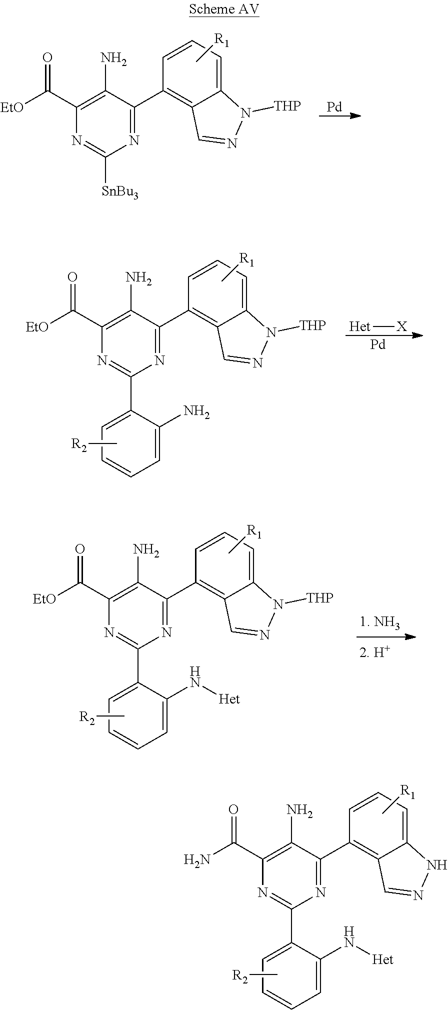

### Method AW

Compounds of the present invention may be prepared as shown in Scheme AW and described herein. Commercially available 2-halogeno-3-bromopyridines bearing substituents can be reacted with primary amines in a SNAr reaction. These 2-amino-3-bromopyridines can then be cross-coupled to stannylated N-THP protected 5-amino-6-(1H-indazol-4-yl)pyrimidine-4-carboxamides bearing substituents on the indazole moiety using a metal-mediated cross-coupling step. Transamidation with ammonia followed by cleavage of the THP group under acidic conditions can then provide compounds of the present invention.

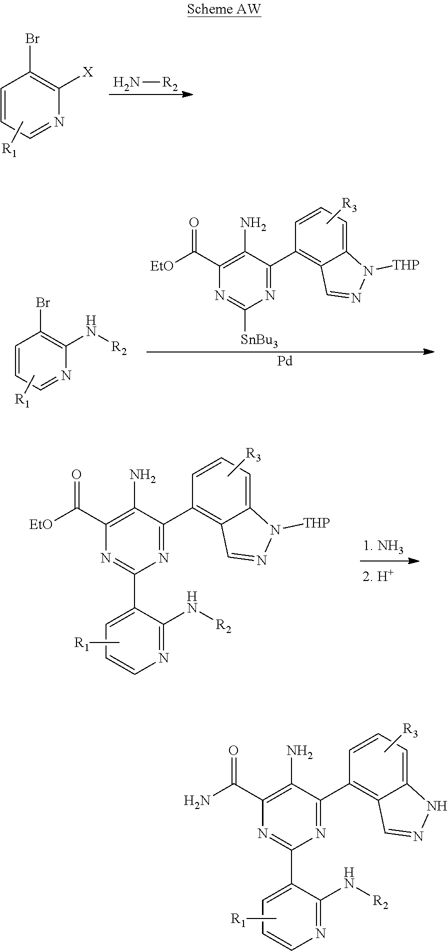

### Method AX

Compounds of the present invention may be prepared as shown in Scheme AX and described herein. N-Alkylated or N-protected 3-amino-4-bromopyrazoles can first be reacted with ketones under reductive amination condition and subsequently borylated with bis(pinacolato)diboron using a metal-mediated cross-coupling step to obtain 4-borylated pyrazoles. These pyrazoles can undergo a second metal-mediated cross-coupling step with 4-chloropyrimidines such as Intermediate AB followed by transamidation with ammonia and cleavage of the THP group under acidic conditions to provide compounds of the present invention.

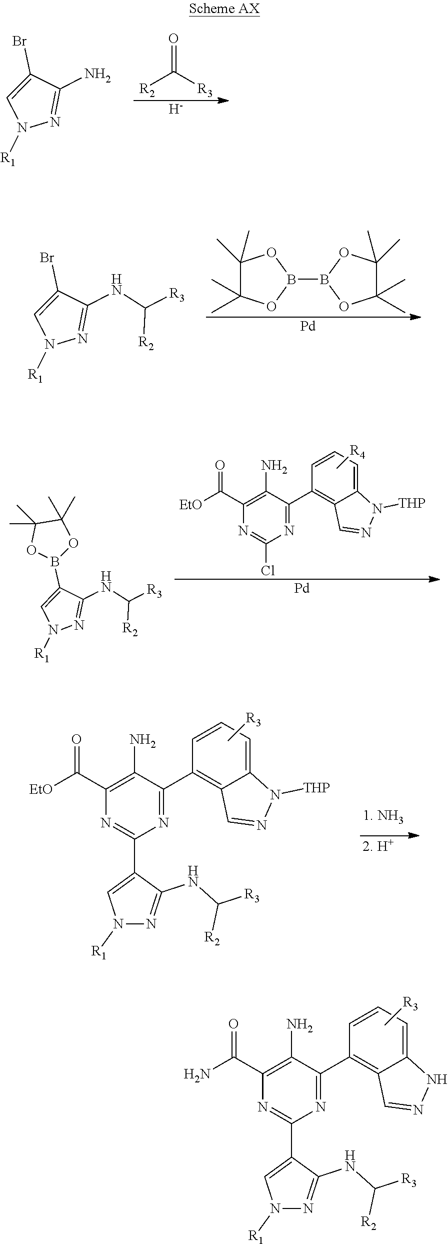

### Method AY

Compounds of the present invention may be prepared as shown in Scheme AY and described herein. N-Alkylated 3-amino-4-bromopyrazoles can first be borylated with bis(pinacolato)diboron using a metal-mediated cross-coupling step and subsequently cross-coupled to 4-chloropyrimidines such as Intermediate AB using a second metal-mediated cross-coupling step. These primary 3-aminopyrazoles can then be N-arylated with an heteroaryl halide using a metal-mediated cross-coupling step. Transamidation with ammonia followed by cleavage of the THP group under acidic conditions can then provide compounds of the present invention.

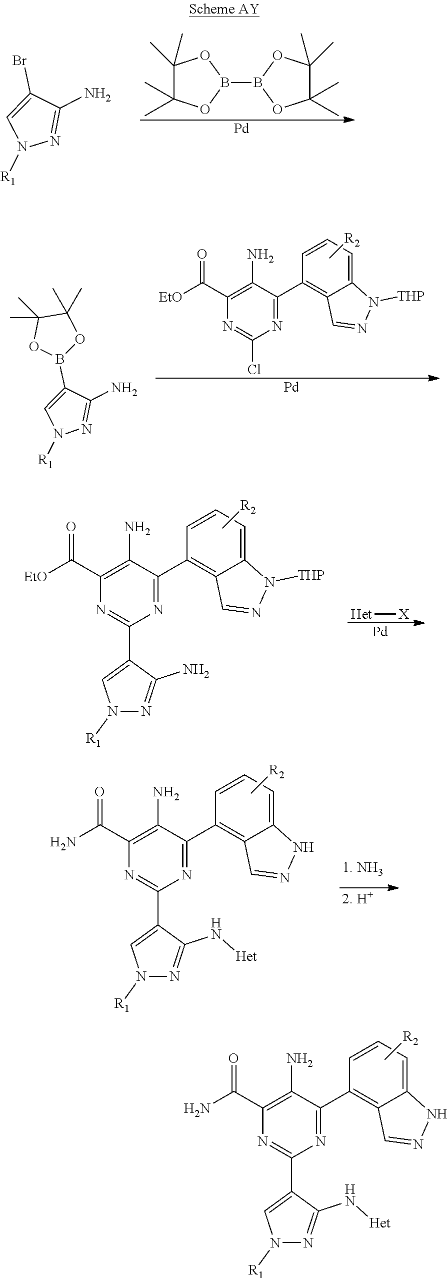

### Method AZ

Compounds of the present invention may be prepared as shown in Scheme AZ and described herein. Intermediate BT can be arylated using a metal-mediated cross-coupling step to install a 3-(2-amino-pyridyl) group which can also bear substitutents. The primary amine can then be N-arylated with heteroaryl halides using a metal-mediated cross-coupling step. Transamidation with ammonia followed by cleavage of the THP group under acidic conditions can then provide compounds of the present invention.

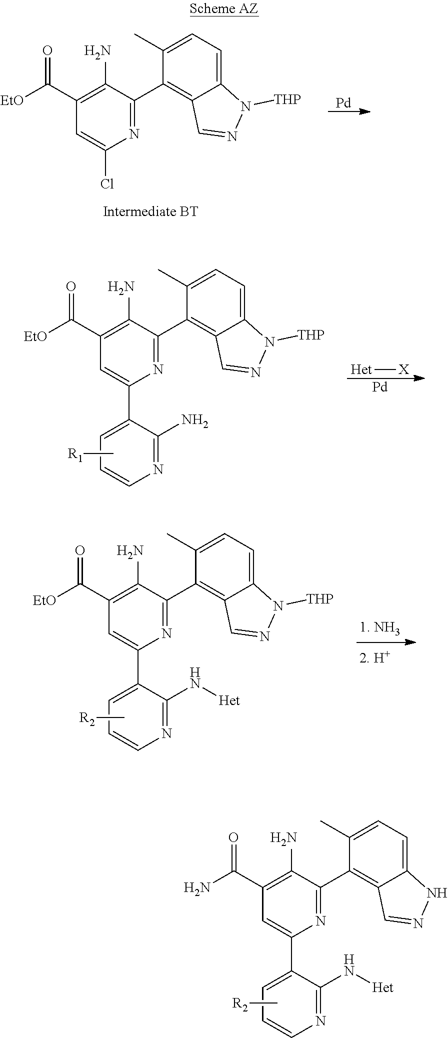

### Method BA

Compounds of the present invention may be prepared as shown in Scheme BA and described herein. Ethyl 5-amino-2-chloro-6-(1-(tetrahydro-2H-pyran-2-yl)-1H-indazol-4-yl)pyrimidine-4-carboxylates bearing substituents on the indazole moiety can by arylated using a metal-mediated cross-coupling step. These arylated intermediates can undergo transamidation with ammonia followed by cleavage of the THP group under acidic conditions to provide compounds of the present invention.

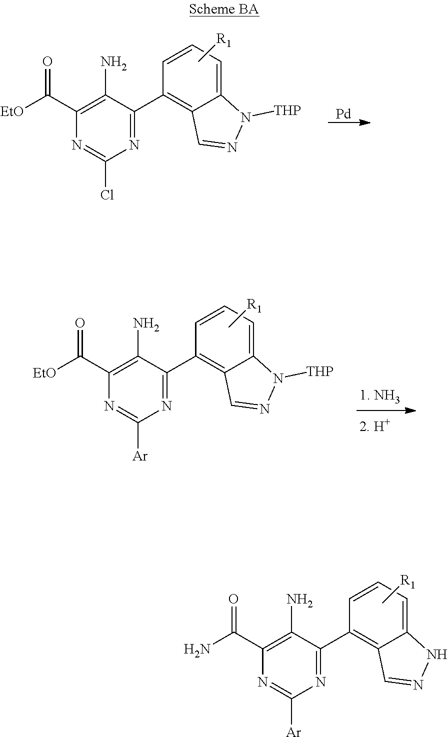

### Method BB

Compounds of the present invention may be prepared as shown in Scheme BB and described herein. Ethyl 5-amino-2-chloro-6-(1-(tetrahydro-2H-pyran-2-yl)-1H-indazol-4-yl)pyrimidine-4-carboxylates bearing substituents on the indazole moiety can by arylated using a metal-mediated cross-coupling step via a one-pot borylation/Suzuki cross-coupling step. The primary amine can then be N-arylated with heteroaryl halides using a metal-mediated cross-coupling step. Transamidation with ammonia followed by cleavage of the THP group under acidic conditions can then provide compounds of the present invention.

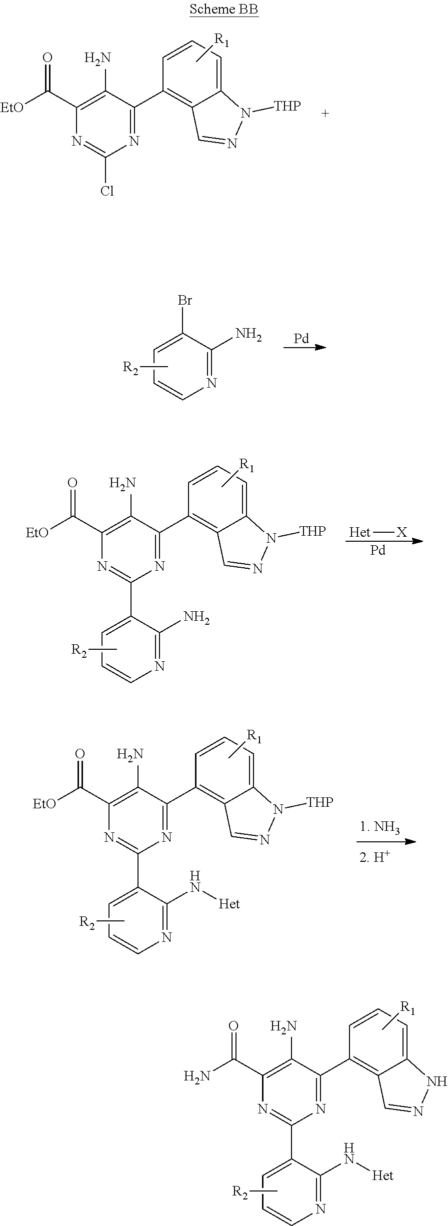

### Method BC

Compounds of the present invention and their synthetic paths to access them have been described in the exemplified compounds below.

### Method BD

Compounds of the present invention may be prepared as shown in Scheme BD and described herein. Intermediate D can be arylated with (3-methoxy-2,6-dimethylphenyl)boronic acid using a metal-mediated cross-coupling step and a subsequent transamidation reaction using ammonia followed by a deprotection with boron tribromide gives Intermediate CO, This intermediate can be arylated using a second metal-mediated cross-coupling step to give compounds of the present invention. Depending on the nature of the aryl group cross-coupled to Intermediate CO, an atropisomeric mixture may be obtained. In such cases it may be necessary to isolate the atropisomer of interest to give compounds of the present invention.

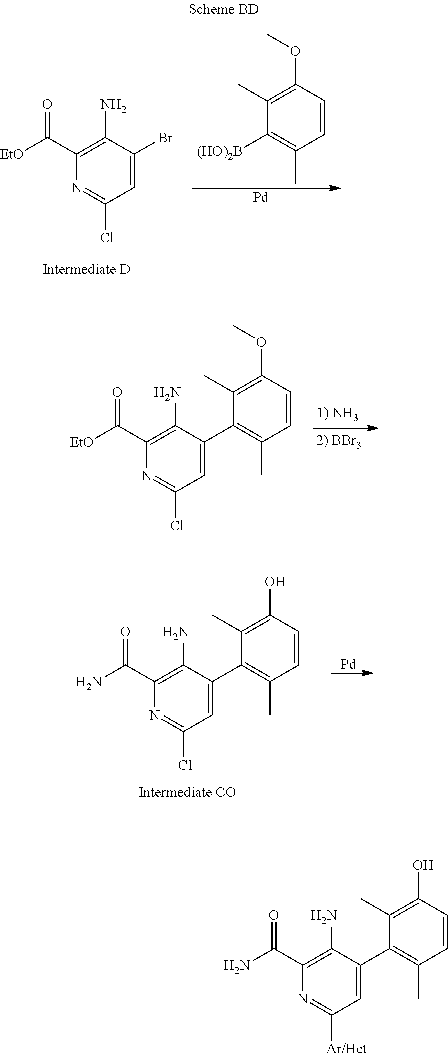

### Methods of Treatment

Compounds of the invention may be used for the treatment of a disease or condition (e.g., a cancer overexpressing CCNE1 or having an inactivating mutation in the FBXW7 gene) which depend on the activity of Myt1 (Gene name PKMYT1).

The disease or condition may have the symptom of cell hyperproliferation. For example, the disease or condition may be a cancer (e.g., a cancer overexpressing CCNE1 or having an inactivating mutation in the FBXW7 gene).

Cancers which have a high incidence of CCNE1 overexpression include e.g., uterine cancer, ovarian cancer, breast cancer, stomach cancer, esophageal cancer, lung cancer, and endometrial cancer.

Cancers which have a deficiency in FBXW7 include, e.g., uterine cancer, colorectal cancer, breast cancer, lung cancer, and esophageal cancer.

A compound of the invention may be administered by a route selected from the group consisting of oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, intratumoral, and topical administration.

### Pharmaceutical Compositions

The compounds used in the methods described herein are preferably formulated into pharmaceutical compositions for administration to human subjects in a biologically compatible form suitable for administration in vivo. Pharmaceutical compositions typically include a compound as described herein and a pharmaceutically acceptable excipient. Certain pharmaceutical compositions may include one or more additional pharmaceutically active agents described herein.

The compounds described herein can also be used in the form of the free base, in the form of salts, zwitterions, solvates, or as prodrugs, or pharmaceutical compositions thereof. All forms are within the scope of the invention. The compounds, salts, zwitterions, solvates, prodrugs, or pharmaceutical compositions thereof, may be administered to a patient in a variety of forms depending on the selected route of administration, as will be understood by those skilled in the art. The compounds used in the methods described herein may be administered, for example, by oral, parenteral, buccal, sublingual, nasal, rectal, patch, pump, or transdermal administration, and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

For human use, a compound of the invention can be administered alone or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers including excipients and auxiliaries that facilitate processing of a compound of the invention into preparations which can be used pharmaceutically.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21st Ed., Gennaro, Ed., Lippincott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6th Edition, Rowe et al., Eds., Pharmaceutical Press (2009).

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21st Ed., Gennaro, Ed., Lippincott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical formulation. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

### Dosages

The dosage of the compound used in the methods described herein, or pharmaceutically acceptable salts or prodrugs thereof, or pharmaceutical compositions thereof, can vary depending on many factors, e.g., the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

A compound of the invention may be administered to the patient in a single dose or in multiple doses. When multiple doses are administered, the doses may be separated from one another by, for example, 1-24 hours, 1-7 days, 1-4 weeks, or 1-12 months. The compound may be administered according to a schedule or the compound may be administered without a predetermined schedule. An active compound may be administered, for example, 1, 2, 3, 4, 5, 6, 7, 8, 9, 10, 11, or 12 times per day, every 2nd, 3rd, 4th, 5th, or 6th day, 1, 2, 3, 4, 5, 6, or 7 times per week, 1, 2, 3, 4, 5, or 6 times per month, or 1, 2, 3, 4, 5, 6, 7, 8, 9, 10, 11, or 12 times per year. It is to be understood that, for any particular subject, specific dosage regimes should be adjusted over time according to the individual need and the professional judgment of the person administering or supervising the administration of the compositions.

While the attending physician ultimately will decide the appropriate amount and dosage regimen, an effective amount of a compound of the invention may be, for example, a total daily dosage of, e.g., between 0.05 mg and 3000 mg of any of the compounds described herein. Alternatively, the dosage amount can be calculated using the body weight of the patient. Such dose ranges may include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

In the methods of the invention, the time period during which multiple doses of a compound of the invention are administered to a patient can vary. For example, in some embodiments, doses of the compounds of the invention are administered to a patient over a time period that is 1-7 days; 1-12 weeks; or 1-3 months. In some embodiments, the compounds are administered to the patient over a time period that is, for example, 4-11 months or 1-30 years. In some embodiments, the compounds are administered to a patient at the onset of symptoms. In any of these embodiments, the amount of compound that is administered may vary during the time period of administration. When a compound is administered daily, administration may occur, for example, 1, 2, 3, 4, 5, 6, 7, 8, 9, 10, 11, or 12 times per day.

### Formulations

A compound identified as capable of treating any of the conditions described herein, using any of the methods described herein, may be administered to patients or animals with a pharmaceutically-acceptable diluent, carrier, or excipient, in unit dosage form. The chemical compounds for use in such therapies may be produced and isolated by any standard technique known to those in the field of medicinal chemistry. Conventional pharmaceutical practice may be employed to provide suitable formulations or compositions to administer the identified compound to patients suffering from a disease or condition. Administration may begin before the patient is symptomatic.

Exemplary routes of administration of the compounds (e.g., a compound of the invention), or pharmaceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. The compounds desirably are administered with a pharmaceutically acceptable carrier. Pharmaceutical formulations of the compounds described herein formulated for treatment of the disorders described herein are also part of the present invention.

### Formulations for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In some embodiments, compositions include biodegradable, pH, and/or temperature-sensitive polymer coatings.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Formulations for Parenteral Administration

The compounds described herein for use in the methods of the invention can be administered in a pharmaceutically acceptable parenteral (e.g., intravenous or intramuscular) formulation as described herein. The pharmaceutical formulation may also be administered parenterally (intravenous, intramuscular, subcutaneous or the like) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:
(1) Drug Injection: a liquid preparation that is a drug substance (e.g., a compound of the invention), or a solution thereof;
(2) Drug for Injection: the drug substance (e.g., a compound of the invention) as a dry solid that will be combined with the appropriate sterile vehicle for parenteral administration as a drug injection;
(3) Drug Injectable Emulsion: a liquid preparation of the drug substance (e.g., a compound of the invention) that is dissolved or dispersed in a suitable emulsion medium;
(4) Drug Injectable Suspension: a liquid preparation of the drug substance (e.g., a compound of the invention) suspended in a suitable liquid medium; and
(5) Drug for Injectable Suspension: the drug substance (e.g., a compound of the invention) as a dry solid that will be combined with the appropriate sterile vehicle for parenteral administration as a drug injectable suspension.

Formulations for parenteral administration include solutions of the compound prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21st Ed., Gennaro, Ed., Lippincott Williams & Wilkins (2005) and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

### Combinations

Compounds of the present invention may be administered to a subject in combination with one or more additional agents, e.g.:
(a) a cytotoxic agent;
(b) an antimetabolite;
(c) an alkylating agent;
(d) an anthracycline;
(e) an antibiotic;
(f) an anti-mitotic agent;
(g) a hormone therapy;
(h) a signal transduction inhibitor;
(i) a gene expression modulator;
(j) an apoptosis inducer;
(k) an angiogenesis inhibitor;
(l) an immunotherapy agent;
(m) a DNA damage repair inhibitor;
or
a combination thereof.

The cytotoxic agent may be, e.g., actinomycin-D, alemtuzumab, alitretinoin, allopurinol, altretamine, amifostine, amphotericin, amsacrine, arsenic trioxide, asparaginase, azacitidine, azathioprine, Bacille Calmette-Guerin (BCG), bendamustine, bexarotene, bevacuzimab, bleomycin, bortezomib, busulphan, capecitabine, carboplatin, carfilzomib, carmustine, cetuximab, cisplatin, chlorambucil, cladribine, clofarabine, colchicine, crisantaspase, cyclophosphamide, cyclosporine, cytarabine, cytochalasin B, dacarbazine, dactinomycin, darbepoetin alfa, dasatinib, daunorubicin, 1-dehydrotestosterone, denileukin, dexamethasone, dexrazoxane, dihydroxy anthracin dione, disulfiram, docetaxel, doxorubicin, emetine, epirubicin, erlotinib, epigallocatechin gallate, epoetin alfa, estramustine, ethidium bromide, etoposide, everolimus, filgrastim, finasunate, floxuridine, fludarabine, flurouracil (5-FU), fulvestrant, ganciclovir, geldanamycin, gemcitabine, glucocorticoids, gramicidin D, histrelin acetate, hydroxyurea, ibritumomab, idarubicin, ifosfamide, imatinib, irinotecan, interferons, interferon alfa-2a, interferon alfa-2b, ixabepilone, lactate dehydrogenase A (LDH-A), lenalidomide, letrozole, leucovorin, levamisole, lidocaine, lomustine, mechlorethamine, melphalan, 6-mercaptopurine, mesna, methotrexate, methoxsalen, metoprine, metronidazole, mithramycin, mitomycin-C, mitoxantrone, nandrolone, nelarabine, nilotinib, nofetumomab, oprelvekin, oxaliplatin, paclitaxel, pemetrexed, pentostatin, palifermin, pamidronate, pegademase, pegaspargase, pegfilgrastim, pemetrexed disodium, plicamycin, porfimer sodium, procaine, procarbazine, propranolol, puromycin, quinacrine, radicicol, radioactive isotopes, raltitrexed, rapamycin, rasburicase, salinosporamide A, sargramostim, sunitinib, temozolomide, teniposide, tetracaine, 6-thioguanine, thiotepa, topotecan, toremifene, trastuzumab, treosulfan, tretinoin, valrubicin, vinblastine, vincristine, vindesine, vinorelbine, zoledronate, or a combination thereof.

The antimetabolites may be, e.g., methotrexate, 6-mercaptopurine, 6-thioguanine, cytarabine, 5-fluorouracil decarbazine, cladribine, pemetrexed, gemcitabine, capecitabine, hydroxyurea, mercaptopurine, fludarabine, pralatrexate, clofarabine, cytarabine, decitabine, floxuridine, nelarabine, trimetrexate, thioguanine, pentostatin, or a combination thereof.

The alkylating agent may be, e.g., mechlorethamine, thiotepa, chlorambucil, melphalan, carmustine (BSNU), lomustine (CCNU), cyclothosphamide, busulfan, dibromomannitol, streptozotocin, mitomycin C, cis-dichlorodiamine platinum (II) (DDP) cisplatin, altretamine, cyclophosphamide, ifosfamide, hexamethylmelamine, altretamine, procarbazine, dacarbazine, temozolomide, streptozocin, carboplatin, cisplatin, oxaliplatin, uramustine, bendamustine, trabectedin, semustine, or a combination thereof.

The anthracycline may be, e.g., daunorubicin, doxorubicin, aclarubicin, aldoxorubicin, amrubicin, annamycin, carubicin, epirubicin, idarubicin, mitoxantrone, valrubicin, or a combination thereof.

The antibiotic may be, e.g., dactinomycin, bleomycin, mithramycin, anthramycin (AMC), ampicillin, bacampicillin, carbenicillin, cloxacillin, dicloxacillin, flucloxacillin, mezlocillin, nafcillin, oxacillin, piperacillin, pivampicillin, pivmecillinam, ticarcillin, aztreonam, imipenem, doripenem, ertapenem, meropenem, cephalosporins, clarithromycin, dirithromycin, roxithromycin, telithromycin, lincomycin, pristinamycin, quinupristin, amikacin, gentamicin, kanamycin, neomycin, netilmicin, paromomycin, tobramycin, streptomycin, sulfamethizole, sulfamethoxazole, sulfisoxazole, demeclocycline, minocycline, oxytetracycline, tetracycline, penicillin, amoxicillin, cephalexin, erythromycin, clarithromycin, azithromycin, ciprofloxacin, levofloxacin, ofloxacin, doxycycline, clindamycin, metronidazole, tigecycline, chloramphenicol, metronidazole, tinidazole, nitrofurantoin, vancomycin, teicoplanin, telavancin, linezolid, cycloserine, rifamycins, polymyxin B, bacitracin, viomycin, capreomycin, quinolones, daunorubicin, doxorubicin, 4′-deoxydoxorubicin, epirubicin, idarubicin, plicamycin, mitomycin-c, mitoxantrone, or a combination thereof.

The anti-mitotic agent may be, e.g., vincristine, vinblastine, vinorelbine, docetaxel, estramustine, ixabepilone, paclitaxel, maytansinoid, a dolastatin, a cryptophycin, or a combination thereof.

The signal transduction inhibitor may be, e.g., imatinib, trastuzumab, erlotinib, sorafenib, sunitinib, temsirolimus, vemurafenib, lapatinib, bortezomib, cetuximab panitumumab, matuzumab, gefitinib, STI 571, rapamycin, flavopiridol, imatinib mesylate, vatalanib, semaxinib, motesanib, axitinib, afatinib, bosutinib, crizotinib, cabozantinib, dasatinib, entrectinib, pazopanib, lapatinib, vandetanib, or a combination thereof.

The gene expression modulator may be, e.g., a siRNA, a shRNA, an antisense oligonucleotide, an HDAC inhibitor, or a combination thereof. An HDAC inhibitor may be, e.g., trichostatin A, trapoxin B, valproic acid, vorinostat, belinostat, LAQ824, panobinostat, entinostat, tacedinaline, mocetionstat, givinostat, resminostat, abexinostat, quisinostat, rocilinostat, practinostat, CHR-3996, butyric acid, phenylbutyric acid, 4SC202, romidepsin, sirtinol, cambinol, EX-527, nicotinamide, or a combination thereof. An antisense oligonucleotide may be, e.g., custirsen, apatorsen, AZD9150, trabadersen, EZN-2968, LErafAON-ETU, or a combination thereof. An siRNA may be, e.g., ALN-VSP, CALAA-01, Atu-027, SPC2996, or a combination thereof.

The hormone therapy may be, e.g., a luteinizing hormone-releasing hormone (LHRH) antagonist. The hormone therapy may be, e.g., firmagon, leuproline, goserelin, buserelin, flutamide, bicalutadmide, ketoconazole, aminoglutethimide, prednisone, hydroxyl-progesterone caproate, medroxy-progesterone acetate, megestrol acetate, diethylstil-bestrol, ethinyl estradiol, tamoxifen, testosterone propionate, fluoxymesterone, flutamide, raloxifene, droloxifene, iodoxyfene, 4-hydroxytamoxifen, trioxifene, keoxifene, LY117018, onapristone, toremifine citrate, megestrol acetate, exemestane, fadrozole, vorozole, letrozole, anastrozole, nilutamide, tripterelin, histerelin, arbiraterone, medroxyprogesterone acetate, diethylstilbestrol, premarin, fluoxymesterone, tretinoin, fenretinide, troxacitabine, or a combination thereof.

The apoptosis inducers may be, e.g., a recombinant human TNF-related apoptosis-inducing ligand (TRAIL), camptothecin, bortezomib, etoposide, tamoxifen, or a combination thereof.

The angiogenesis inhibitors may be, e.g., sorafenib, sunitinib, pazopanib, everolimus or a combination thereof.

The immunotherapy agent may be, e.g., a monoclonal antibody, cancer vaccine (e.g., a dendritic cell (DC) vaccine), oncolytic virus, cytokine, adoptive T cell therapy, Bacille Calmette-Guerin (BCG), GM-CSF, thalidomide, lenalidomide, pomalidomide, imiquimod, or a combination thereof. The monoclonal antibody may be, e.g., anti-CTLA4, anti-PD1, anti-PD-L1, anti-LAG3, anti-KIR, or a combination thereof.

The monoclonal antibody may be, e.g., alemtuzumab, trastuzumab, ibritumomab tiuxetan, brentuximab vedotin, trastuzumab, ado-trastuzumab emtansine, blinatumomab, bevacizumab, cetuximab, pertuzumab, panitumumab, ramucirumab, obinutuzumab, ofatumumab, rituximab, pertuzumab, tositumomab, gemtuzumab ozogamicin, tositumomab, or a combination thereof. The cancer vaccine may be, e.g., Sipuleucel-T, BioVaxID, NeuVax, DCVax, SuVaxM, CIMAvax@, Provenge,®, hsp110 chaperone complex vaccine, CDX-1401, MIS416, CDX-110, GVAX Pancreas, HyperAcute™ Pancreas, GTOP-99 (MyVax®), or Imprime PGG®. The oncolytic virus may be, e.g., talimogene laherparepvec. The cytokine may be, e.g., IL-2, IFNα, or a combination thereof. The adoptive T cell therapy may be, e.g., tisagenlecleucel, axicabtagene ciloleucel, or a combination thereof.

The DNA damage repair inhibitor may be, e.g., a PARP inhibitor, a cell checkpoint kinase inhibitor, or a combination thereof. The PARP inhibitor may be, e.g., olaparib, rucaparib, veliparib (ABT-888), niraparib (ZL-2306), iniparib (BSI-201), talazoparib (BMN 673), 2X-121, CEP-9722, KU-0059436 (AZD2281), PF-01367338 or a combination thereof. The cell checkpoint kinase inhibitor may be, e.g., MK-1775 or AZD1775, AZD7762, LY2606368, PF-0477736, AZD0156, GDC-0575, ARRY-575, CCT245737, PNT-737 or a combination thereof.

### EXAMPLES

The following examples were meant to illustrate the invention. They were not meant to limit the invention in any way.

Reactions were typically performed at room temperature (rt or RT) under a nitrogen atmosphere using dry solvents (Sure/Seal™) if not described otherwise in the Examples below. Reactions were monitored by TLC or by injection of a small aliquot on a Waters Acquity-H UPLC® Class system using an Acquity® UPLC HSS C18 2.1×30 mm column eluting with a gradient (1.86 min) of acetonitrile (15% to 98%) in water (both containing 0.1% formic acid). Purifications by preparative HPLC were performed on a Teledyne Isco Combi Flash® EZ Prep system using either Phenomenex Gemini® 5 μm NX-C18 110A 150×21.2 mm column at a flow of 40 mL/min over 12 min (<100 mg or multiple injections of <100 mg) or HP C18 RediSep® Rf gold column (>100 mg) eluting with an appropriate gradient of acetonitrile in water (both containing 0.1% formic acid) unless otherwise specified. The gradient was selected based on the retention time observed by reaction monitoring on the Waters Acquity-H UPLC® Class system (see above). Fractions containing the desired compounds were combined and finally lyophilized. Purifications by silica gel chromatography were performed on a Teledyne Isco Combi Flash® Rf system using RediSep® Rf silica gel columns of appropriate sizes. Purity of final Compounds was assessed by injection of a small aliquot on a Waters Acquity-H UPLC® Class system using an Acquity® UPLC BEH C18 2.1×50 mm column eluting with a gradient (7 min) of acetonitrile (2% to 98%) in water (both containing 0.1% formic acid).

### Example 1. Preparation of Compounds

### Intermediates

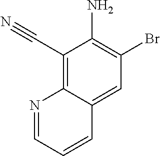 [SMILES: N#Cc1c(N)c(Br)cc2cccnc12]

### Intermediate A (7-amino-6-bromoquinoline-8-carbonitrile)

Step 1. A microwave reaction tube was charged with 8-bromoquinolin-7-amine (100 mg, 448.3 μmol), copper cyanide (50 mg, 558 μmol) and NMP (2 mL). Nitrogen was bubbled into the reaction solution for 10 minutes, and the mixture was heated at 170° C. for 1.5 h in a microwave reactor. The reaction mixture was purified by prep HPLC to provide 7-aminoquinoline-8-carbonitrile (33 mg, 44% yield). MS: [M+H]+: 170.2.

Step 2. To 7-aminoquinoline-8-carbonitrile (40 mg, 236 μmol) in ACN (3 mL) was added acetic acid (150 μL) and N-bromosuccinimide (55 mg, 307 μmol). The mixture was stirred for 17 h at 90° C. The solvents were removed under reduced pressure. The residue was purified by silica gel chromatography eluting with a gradient of 0 to 95% EtOAc in hexanes to provide 7-amino-6-bromo-quinoline-8-carbonitrile (15 mg, 26% yield). MS: [M+H]+: 248.0.

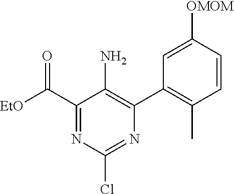

### Intermediate B (ethyl 5-amino-2-chloro-6-(5-(methoxymethoxy)-2-methylphenyl)pyrimidine-4-carboxylate)

To a solution of ethyl 5-amino-2,6-dichloro-pyrimidine-4-carboxylate (800 mg, 3.4 mmol) in dioxane (9 mL) were added [5-(methoxymethoxy)-2-methyl-phenyl]boronic acid (680 mg, 3.5 mmol), Pd(PPh₃)₄ (200 mg, 173 μmol) and 2 M aqueous solution of sodium carbonate (3.2 mL, 6.4 mmol). The mixture was degassed in vacuo and then back-filled with N₂ and then stirred at 100° C. for 5 h. The mixture was diluted with EtOAc (80 mL), washed with water and brine consecutively, dried over sodium sulfate, filtered and concentrated to dryness. The residue was purified using flash silica gel chromatography eluting with EtOAc/hexanes 0-40% to provide ethyl 5-amino-2-chloro-6-(5-(methoxymethoxy)-2-methylphenyl)pyrimidine-4-carboxylate (760 mg, 64% yield). MS: [M+H]+: 352.2; ¹H NMR (400 MHz, Chloroform-d) δ 7.31-7.16 (m, 1H), 7.02 m, 1H), 6.92 (m, 1H), 5.71 (s, 2H), 5.11 (s, 2H), 4.44 (q, J=7.1 Hz, 2H), 3.41 (s, 3H), 2.09 (s, 3H), 1.41 (t, J=7.1 Hz, 3H).

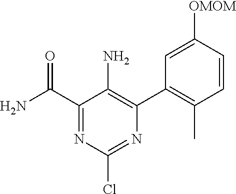

### Intermediate C (5-amino-2-chloro-6-(5-(methoxymethoxy)-2-methylphenyl)pyrimidine-4-carboxamide)

Step 1. To a solution of Intermediate B (760 mg, 2.16 mmol) in MeOH (3 mL) and THF (10 mL) was added 1 M aqueous solution of sodium hydroxide (3 mL, 3 mmol). The mixture was stirred at rt for 2 h. Then it was acidified to pH 4 using 1 M aqueous solution of HCl and extracted with EtOAc 3×. The combined organic extracts was washed with brine, dried over sodium sulfate, filtered and concentrated to provide 5-amino-2-chloro-6-[5-(methoxymethoxy)-2-methyl-phenyl]pyrimidine-4-carboxylic acid (590 mg, 84% yield). MS: [M+H]+: 320.2.

Step 2. To a solution of 5-amino-2-chloro-6-[5-(methoxymethoxy)-2-methyl-phenyl]pyrimidine-4-carboxylic acid (520 mg, 1.61 mmol) in DMF (20 mL) were added HATU (780 mg, 2.05 mmol), 0.5 M solution of ammonia in dioxane (10 mL, 5 mmol) and triethylamine (330 μL, 2.37 mmol). The mixture was stirred at 50° C. for 1 h. The mixture was concentrated to a small volume in vacuo then diluted with water, stirred at rt for 20 min, filtered. The solid was washed with water, dried in vacuo to obtain 5-amino-2-chloro-6-(5-(methoxymethoxy)-2-methylphenyl)pyrimidine-4-carboxamide (420 mg, 81% yield).

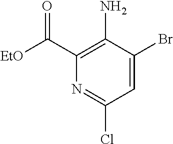 [SMILES: CCOC(=O)c1nc(Cl)cc(Br)c1N]

### Intermediate D (ethyl 3-amino-4-bromo-6-chloropicolinate)

Step 1. To a solution of 3-amino-6-chloro-pyridine-2-carboxylic acid (60.0 g, 347.7 mmol) in DMF (650 mL) was added K₂CO₃ (50.6 g, 366.11 mmol), iodoethane (31 mL, 385.60 mmol) and tetrabutylammonium iodide (579 mg, 1.74 mmol). The mixture was stirred overnight at rt then transferred to a 4 L conical flask and cooled in ice bath. The mixture was diluted with water (2 L) and stirred for 90 min. The solids were collected by filtration, washed with water and dried in vacuo to afford ethyl 3-amino-6-chloro-pyridine-2-carboxylate (61.5 g, 88% yield). MS: [M+H]+: 201.1.

Step 2. To a solution of ethyl 3-amino-6-chloro-pyridine-2-carboxylate (13.08 g, 65.2 mmol) in DMF (130 mL) was added NBS (15.0 g, 84.3 mmol). The mixture was stirred overnight at rt. The mixture was cooled in an ice bath and water (260 mL) was added dropwise. The solid was collected by filtration on Buchner. This solid was suspended in 20% wt aqueous solution of Na₂S₂O₃ (60 mL) and saturated aqueous NaHCO₃ solution (130 mL) and stirred at rt for about 30 min. The solid was collected by filtration on Buchner, washed with water then dried in vacuo, affording ethyl 3-amino-4-bromo-6-chloro-pyridine-2-carboxylate (13.68 g, 75% yield). MS: [M+H]+: 278.9. ¹H NMR (400 MHz, Chloroform-d) δ 7.54 (s, 1H), 6.36 (s, 2H), 4.43 (q, J=7.1 Hz, 2H), 1.41 (t, J=7.1 Hz, 3H).

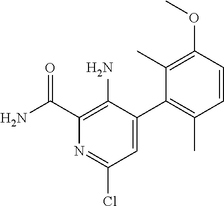 [SMILES: COc1ccc(C)c(-c2cc(Cl)nc(C(N)=O)c2N)c1C]

### Intermediate E (3-amino-6-chloro-4-(3-methoxy-2,6-dimethylphenyl)picolinamide)

Step 1. To a solution of Intermediate D (6.0 g, 21.5 mmol) in toluene (100 mL) were added 2-(3-methoxy-2,6-dimethyl-phenyl)-4,4,5,5-tetramethyl-1,3,2-dioxaborolane (6.00 g, 22.9 mmol), 2M aqueous solution of Na₂CO₃ (21 mL, 42 mmol), tri-tert-butylphosphine 1 M in toluene (4.2 mL, 4.20 mmol) and Pd₂(dba)₃ (1.95 g, 2.13 mmol). The mixture was degassed in vacuo and then back-filled with N₂ then stirred at 90° C. for 2 h. The volatiles were removed in vacuo and the residue was purified using flash silica gel chromatography eluting with EtOAc/hexanes 0-30% to provide ethyl 3-amino-6-chloro-4-(3-methoxy-2,6-dimethyl-phenyl)pyridine-2-carboxylate (4.56 g, 63% yield). MS: [M+H]+: 335.2.

Step 2. A 150 mL pressure vessel was charged with ethyl 3-amino-6-chloro-4-(3-methoxy-2,6-dimethyl-phenyl)pyridine-2-carboxylate (1.45 g, 4.32 mmol) and 7 N ammonia solution in MeOH (50 mL, 350 mmol). The vessel was sealed and stirred at 70° C. overnight. The mixture was cooled to rt then concentrated to dryness. The residue was purified by flash silica gel chromatography eluting with EtOAc/hexanes 0-50% to provide 3-amino-6-chloro-4-(3-methoxy-2,6-dimethyl-phenyl)pyridine-2-carboxamide (1.13 g, 86% yield). MS: [M+H]+: 306.2.

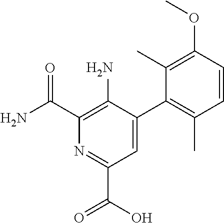 [SMILES: COc1ccc(C)c(-c2cc(C(=O)O)nc(C(N)=O)c2N)c1C]

### Intermediate F (5-amino-6-carbamoyl-4-(3-methoxy-2,6-dimethylphenyl)picolinic acid)

Step 1. A MW vial was charged with 3-amino-6-chloro-4-(3-methoxy-2,6-dimethyl-phenyl)pyridine-2-carboxamide (500 mg, 1.64 mmol), 2-furylboronic acid (300 mg, 2.68 mmol) and Pd(dppf)Cl₂·DCM (133 mg, 162.86 μmol). DMF (9 mL) and 2 M aqueous solution of sodium carbonate (2.0 mL, 4.0 mmol) are added and the solution was bubbled through with N₂, capped and transferred to a preheated (120° C.) heat block for 1 h. The mixture was cooled to rt, filtered through a Celite™ cartridge, washed with portions of EtOAc (50 mL total) and water (25 mL total) then diluted with saturate NH₄Cl (25 mL). The layers were separated and the aqueous layer was extracted with EtOAc (2×25 mL). The combined organic extracts was washed with brine (25 mL), dried over Na₂SO₄, filtered and concentrated. The residue was purified using flash silica gel chromatography eluting with EtOAc/hexanes 0-100% to provide 3-amino-6-(2-furyl)-4-(3-methoxy-2,6-dimethyl-phenyl)pyridine-2-carboxamide (544 mg, 99% yield). MS: [M+H]+: 338.3.

Step 2. To a suspension of 3-amino-6-(2-furyl)-4-(3-methoxy-2,6-dimethyl-phenyl)pyridine-2-carboxamide (575 mg, 1.70 mmol) in t-BuOH (10 mL) and water (2.5 mL) was added potassium permanganate (1.34 g, 8.49 mmol) in one portion. The solution was sonicated then stirred at rt. After 75 min, the mixture was filtered on Celite™, washed with MeOH and concentrated. The residue was purified by reverse-phase flash chromatography (MeCN in water, both containing 0.1% formic acid) with 10-80% to provide 5-amino-6-carbamoyl-4-(3-methoxy-2,6-dimethyl-phenyl)pyridine-2-carboxylic acid (140 mg, 26% yield). MS: [M+H]+: 316.3.

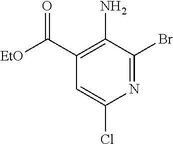 [SMILES: CCOC(=O)c1cc(Cl)nc(Br)c1N]

### Intermediate G (ethyl 3-amino-2-bromo-6-chloroisonicotinate)

Step 1. To a solution of 5-amino-2-chloro-pyridine-4-carboxylic acid (5 g, 29.0 mmol) in DMF (55 mL) was added K₂CO₃ (4.40 g, 31.9 mmol) and iodoethane (2.5 mL, 31.1 mmol). The mixture was stirred overnight at rt for 18 h. 0.2 eq of K₂CO₃ (0.8 g, 5.8 mmol) and 0.2 eq of idoethane (465 μL, 5.8 mmol) were added and the mixture was stirred for another 24 h at rt. The mixture was slowly diluted with water (2 volume) and stirred for 30 min. The solids were collected by filtration on Buchner, washed with water and air-dried overnight to yield ethyl 5-amino-2-chloro-pyridine-4-carboxylate (4.4 g, 76% yield). MS: [M+H]+: 201.1.

Step 2. To a solution of ethyl 5-amino-2-chloro-pyridine-4-carboxylate (4.4 g, 21.9 mmol) in DMF (50 mL) was added NBS (4.7 g, 26.4 mmol). The mixture was stirred 2 h at rt. Water (2 volume) was slowly added and the precipitate was recovered by filtration to yield ethyl 3-amino-2-bromo-6-chloroisonicotinate (5.1 g, 83% yield). MS: [M+H]⁺: 278.9; 280.9; 282.9.

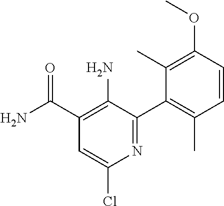 [SMILES: COc1ccc(C)c(-c2nc(Cl)cc(C(N)=O)c2N)c1C]

### Intermediate H (3-amino-6-chloro-2-(3-methoxy-2,6-dimethylphenyl)isonicotinamide)

Step 1. To the solution of Intermediate G (1.0 g, 3.58 mmol) in dioxane (20 mL) were added 2-(3-methoxy-2,6-dimethyl-phenyl)-4,4,5,5-tetramethyl-1,3,2-dioxaborolane (1.05 g, 4.01 mmol), Pd₂(dba)₃ (330 mg, 360.37 μmol) and 2 M aqueous solution of K₃PO₄ (3.6 mL, 7.2 mmol). The mixture was degassed in vacuo and then back-filled with N2. Tri-tert-butylphosphonium tetrafluoroborate (210 mg, 723.8 μmol) was added and the mixture was degassed two more time and then stirred at 90° C. for 2 h. The volatiles were removed in vacuo and the residue was purified using flash chromatography eluting with EtOAc/hexanes 0-30% to provide ethyl 3-amino-6-chloro-2-(3-methoxy-2,6-dimethyl-phenyl)pyridine-4-carboxylate (1.05 g, 88% yield). MS: [M+H]+: 335.2.

Step 2. 7 N solution of ammonia in MeOH (10 mL, 70 mmol) was added to ethyl 3-amino-6-chloro-2-(3-methoxy-2,6-dimethyl-phenyl)pyridine-4-carboxylate (500 mg, 1.49 mmol) in a Parr pressure vessel. The vessel was sealed with teflon and heated to 130° C. for 4 h. After cooling to RT, the solution was concentrated to dryness. The residue was purified by silica gel chromatography eluting with a gradient of EtOAc (0 to 100%) in hexanes to provide 3-amino-6-chloro-2-(3-methoxy-2,6-dimethyl-phenyl)pyridine-4-carboxamide (200 mg, 44% yield) and methyl 3-amino-6-chloro-2-(3-methoxy-2,6-dimethyl-phenyl)pyridine-4-carboxylate (175 mg, 37%). MS: [M+H]⁺: 306.2.

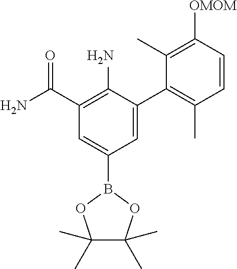

### Intermediate I (2-amino-3′-(methoxymethoxy)-2′,6′-dimethyl-5-(4,4,5,5-tetramethyl-1,3,2-dioxaborolan-2-yl)-[1,1′-biphenyl]-3-carboxamide)

Step 1. A pressure vessel was charged with 2-amino-5-bromo-3-iodobenzamide (0.5 g, 1.47 mmol), dioxane (5 mL), water (1 mL), Na₂CO₃ (0.233 g, 2.19 mmol) and (3-(methoxymethoxy)-2,6-dimethylphenyl)boronic acid (0.436 g, 1.49 mmol). The mixture was purged with N₂ for 10 min, followed by addition of Pd(PPh₃)₄ (0.084 g, 0.073 mmol). The vessel was sealed and the mixture was stirred at 80° C. for 5 h. The reaction mixture was quenched in water (20 mL) and extracted with EtOAc (3×10 mL). The combined organic layer was dried over Na₂SO₄, filtered and concentrated. The residue was purified by silica gel chromatography eluting with 35% EtOAc in hexanes to provide 2-amino-5-bromo-3′-(methoxymethoxy)-2′,6′-dimethyl-[1,1′-biphenyl]-3-carboxamide (0.107 g, 16%). MS: [M+H]⁺: 379.4

Step 2. A pressure vessel was charged with 2-amino-5-bromo-3′-(methoxymethoxy)-2′,6′-dimethyl-[1,1′-biphenyl]-3-carboxamide (0.1 g, 0.264 mmol), dioxane (3 mL), Cs₂CO₃ (0.215 g, 0.659 mmol) and bis(pinacolato)diboron (0.133 g, 0.527 mmol). The vessel was purged with N2 gas for 10 min, followed by addition of PdCl2(dppf) (0.038 g, 0.0527 mmol). The vessel was sealed and the reaction mixture was stirred at 100° C. for 2 h. The reaction mixture was quenched with water (10 mL) and extracted with EtOAc (3×10 mL). The combined organic layer was dried over Na₂SO₄, filtered and concentrated. The residue was purified by silica gel chromatography eluting with 35% EtOAc in hexanes to provide 2-amino-3′-(methoxymethoxy)-2′,6′-dimethyl-5-(4,4,5,5-tetramethyl-1,3,2-dioxaborolan-2-yl)-[1,1′-biphenyl]-3-carboxamide. (0.08 g, 71%). MS: [M+H]⁺: 427.2.4

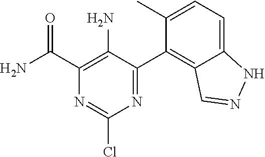 [SMILES: Cc1ccc2[nH]ncc2c1-c1nc(Cl)nc(C(N)=O)c1N]

### Intermediate J (5-amino-2-chloro-6-(5-methyl-1H-indazol-4-yl)pyrimidine-4-carboxamide)

Step 1. A mixture of ethyl 5-amino-2,6-dichloro-pyrimidine-4-carboxylate (4.92 g, 20.84 mmol), (5-methyl-1H-indazol-4-yl)boronic acid (3.67 g, 20.84 mmol) and Na₂CO₃ (3.53 g, 33.35 mmol) in dioxane (50 mL) and water (5 mL) was degassed by bubbling N₂, then Pd(PPh₃)₄ (1.44 g, 1.25 mmol) was added. The solution was degassed again then heated at 80° C. overnight. The reaction mixture was cooled to rt, poured in water (100 mL) and extracted with EtOAc (2×50 mL). The combined organic layers were washed with brine, dried over Na₂SO₄, filtered and concentrated in vacuo. The residue was purified by silica gel chromatography eluting with a gradient of EtOAc (0 to 100%) in heptane to afford ethyl 5-amino-2-chloro-6-(5-methyl-1H-indazol-4-yl)pyrimidine-4-carboxylate (3.86 g, 56% yield). MS: [M+H]⁺: 332.0.

Step 2. To a suspension of ethyl 5-amino-2-chloro-6-(5-methyl-1H-indazol-4-yl)pyrimidine-4-carboxylate (3.86 g, 11.64 mmol) in THF (17 mL) and MeOH (17 mL) was added 4 M aqueous sodium hydroxide aqueous (28 mL, 112 mmol). After stirring for 70 min, the pH was adjusted to 4-5 with 3 N aqueous HCl and diluted with water (50 mL). The resulting suspension was stirred for 2 h at 0° C. and the solid was collected by filtration and washed with water. The solid was air-dried overnight to provide 5-amino-2-chloro-6-(5-methyl-1H-indazol-4-yl)pyrimidine-4-carboxylic acid (2.73 g, 77% yield). MS: [M+H]⁺: 304.0.

Step 3. A pressure vessel was charged with 5-amino-2-chloro-6-(5-methyl-1H-indazol-4-yl)pyrimidine-4-carboxylic acid (2.62 g, 8.63 mmol), ammonium chloride (2.31 g, 43.10 mmol) and HATU (3.90 g, 10.27 mmol). DMF (27 mL) was added followed by DIPEA (9.2 mL, 52.82 mmol). The vessel was sealed and stirred at 80° C. for 75 min. Water was slowly added to the cooled reaction mixture under stirring. The precipitate was collected by filtration, washed with water and air-dried overnight to provide 5-amino-2-chloro-6-(5-methyl-1H-indazol-4-yl)pyrimidine-4-carboxamide (2.19 g, 84% yield). MS: [M+H]⁺: 303.0; ¹H NMR (400 MHz, DMSO-d6) δ 13.16 (s, 1H), 8.23 (s, 1H), 7.89 (s, 1H), 7.69 (s, 1H), 7.60 (d, J=8.5 Hz, 1H), 7.35 (d, J=8.5 Hz, 1H), 6.50 (br s, 2H), 2.20 (s, 3H).

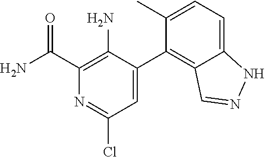 [SMILES: Cc1ccc2[nH]ncc2c1-c1cc(Cl)nc(C(N)=O)c1N]

Intermediate K (3-amino-6-chloro-4-(5-methyl-1H-indazol-4-yl)picolinamide) Step 1. To a solution of Intermediate G (30.10 g, 107.7 mmol) and (5-methyl-1H-indazol-4-yl)boronic acid (22.66 g, 128.8 mmol) in dioxane (200 mL) and 2 M aqueous solution of K₃PO₄ (110 mL, 220 mmol) were added Pd₂(dba)₃ (2.50 g, 2.73 mmol) and a 1 M toluene solution of tri-tert-butylphosphine (10.7 mL, 10.7 mmol). The mixture was degassed by bubbling N₂ and stirred at 90° C. for 4.5 h. The cooled reaction mixture was concentrated. Water (600 mL) was added dropwise. The solids were collected by filtration, washed with water, air-dried then and triturated with heptane (250 mL) to provide ethyl 3-amino-6-chloro-4-(5-methyl-1H-indazol-4-yl)pyridine-2-carboxylate (25.63 g, 72% yield). MS: [M+H]⁺: 331.1.

Step 2. Ethyl 3-amino-6-chloro-4-(5-methyl-1H-indazol-4-yl)pyridine-2-carboxylate (9.24 g, 27.9 mmol) in 7 N ammonia solution in MeOH (300 mL, 2.1 mol) was heated to 100° C. overnight in a pressure vessel. The mixture was cooled to rt, concentrated to dryness, coevaporated with DCM/MeOH then with DCM/heptane and dried under vacuum to provide 3-amino-6-chloro-4-(5-methyl-1H-indazol-4-yl)pyridine-2-carboxamide (8.35 g, 99% yield). MS: [M+H]⁺: 302.0

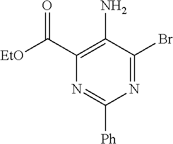 [SMILES: CCOC(=O)c1nc(-c2ccccc2)nc(Br)c1N]

### Intermediate L (ethyl 5-amino-6-bromo-2-phenylpyrimidine-4-carboxylate)

Step 1. A RBF was charged with ethyl 5-amino-2-chloro-pyrimidine-4-carboxylate (3.97 g, 19.7 mmol), phenylboronic acid (3.59 g, 29.4 mmol) and Pd(dppf)Cl₂ (1.43 g, 1.95 mmol). Dioxane (80 mL) and 2 M aqueous solution of Na₂CO₃ (30 mL, 60 mmol) were added and the mixture was bubbled through with N2. The flask was equipped with a condenser and heated to 100° C. for 2 h. The cooled reaction mixture was diluted with water (150 mL) and extracted with DCM (2×50 mL). The combined organic extracts were washed with water and brine (100 mL each), dried over Na₂SO₄, filtered and concentrated. The residue was purified by silica gel chromatography eluting with a gradient of EtOAc (0 to 100%) in heptane to afford ethyl 5-amino-2-phenyl-pyrimidine-4-carboxylate (3.99 g, 83% yield). MS: [M+H]⁺: 244.2.

Step 2. To a solution of ethyl 5-amino-2-phenyl-pyrimidine-4-carboxylate (2.06 g, 8.47 mmol) in DMF (20 mL) was added NBS (2.21 g, 12.4 mmol). The solution was stirred at rt for 45 min. More NBS (292 mg, 1.64 mmol) was added and the solution was stirred at rt for another 90 min. The reaction mixture was diluted with EtOAc (80 mL) and aqueous saturated NaHCO₃ solution (80 mL). The aqueous layer was extracted with EtOAc (2×40 mL) and the combined organic layers was washed with 20% wt aqueous Na₂S₂O₄ solution (40 mL), brine (40 mL), dried over Na₂SO₄, filtered and concentrated. The residue was purified by silica gel chromatography eluting with a gradient of EtOAc (0 to 50%) in heptane to afford ethyl 5-amino-6-bromo-2-phenyl-pyrimidine-4-carboxylate (1.07 g, 39% yield). MS: [M+H]⁺: 324.1; ¹H NMR (400 MHz, Chloroform-d) δ 8.38-8.20 (m, 2H), 7.50-7.35 (m, 3H), 6.22 (br s, 2H), 4.49 (q, J=7.1 Hz, 2H), 1.49 (t, J=7.1 Hz, 3H).

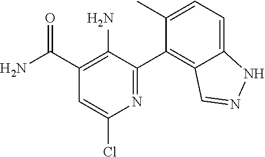 [SMILES: Cc1ccc2[nH]ncc2c1-c1nc(Cl)cc(C(N)=O)c1N]

### Intermediate M (3-amino-6-chloro-2-(5-methyl-1H-indazol-4-yl)isonicotinamide)

To a solution of Intermediate G (5 g, 17.9 mmol) in dioxane (100 mL) were added (5-methyl-1H-indazol-4-yl)boronic acid (3.5 g, 19.9 mmol), Pd₂(dba)₃ (1.65 g, 1.80 mmol) and 2M aqueous solution of K₃PO₄ (18 mL, 36 mmol). The mixture was degassed in vacuo, back-filled with N₂ and then stirred at 90° C. for 2 h. The volatiles were removed in vacuo and the residue was purified by silica gel chromatography eluting with a gradient of EtOAc (0 to 30%) in heptane to provide ethyl 3-amino-6-chloro-2-(5-methyl-1H-indazol-4-yl)pyridine-4-carboxylate (5.3 g, 90% yield). MS: [M+H]⁺: 302.0.

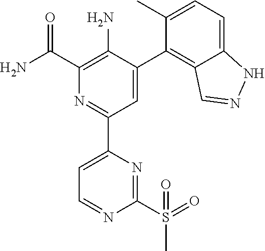 [SMILES: Cc1ccc2[nH]ncc2c1-c1cc(-c2ccnc(S(C)(=O)=O)n2)nc(C(N)=O)c1N]

### Intermediate N (3-amino-4-(5-methyl-1H-indazol-4-yl)-6-(2-(methylsulfonyl)pyrimidin-4-yl)picolinamide)

Step 1. A pressure vessel was charged with Intermediate K (0.39 g, 1.29 mmol) and DMF (4 mL). LiCl (0.5 M in THF) (5.2 mL, 2.38 mmol) was added followed by 2-(methylthio)-4-(tributylstannyl)pyrimidine (0.644 g, 1.68 mmol). The reaction mixture was purged with N2 gas, followed by addition of CuI (0.039 g, 0.192 mmol) and Pd(ddpf)Cl₂·DCM (0.105 g, 0.129 mmol). The resulting mixture was stirred at 120° C. for 4 h. The cooled reaction mixture was quenched in water (30 mL) then extracted with EtOAc (3×20 mL). The combined organic layer was dried over Na₂SO₄, filtered and concentrated. The residue was purified by silica gel chromatography eluting with 2% methanol in dichloromethane to provide 3-amino-4-(5-methyl-1H-indazol-4-yl)-6-(2-(methylthio)pyrimidin-4-yl)picolinamide (0.28 g, 55% yield). MS: [M+H]⁺: 392.2.

Step 2. A pressure vessel was charged with 3-amino-4-(5-methyl-1H-indazol-4-yl)-6-(2-(methylthio)pyrimidin-4-yl)picolinamide (0.28 g, 0.7161 mmol) and THF (3 mL). This solution was added to a stirred solution water (1 mL) and Oxone (0.88 g, 2.864 mmol) portionwise and the reaction mixture was stirred at rt for 2 h. The reaction mixture was quenched with water (30 mL) then extracted with EtOAc (3×20 mL). The combined organic layer was dried over Na₂SO₄, filtered and concentrated. The residue was triturated with n-pentane to provide 3-amino-4-(5-methyl-1H-indazol-4-yl)-6-(2-(methylsulfonyl)pyrimidin-4-yl)picolinamide (0.2 g, 66% yield). MS: [M+H]⁺: 424.5.

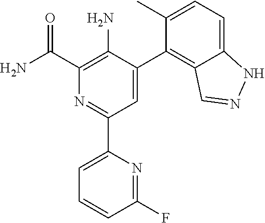 [SMILES: Cc1ccc2[nH]ncc2c1-c1cc(-c2cccc(F)n2)nc(C(N)=O)c1N]

### Intermediate O (5-amino-6′-fluoro-4-(5-methyl-1H-indazol-4-yl)-[2,2′-bipyridine]-6-carboxamide)

A suspension of tributyl-(6-fluoro-2-pyridyl)stannane (3.84 g, 9.94 mmol), 3-amino-6-chloro-4-(5-methyl-1H-indazol-4-yl)pyridine-2-carboxamide (3 g, 9.94 mmol), copper(I) iodide (239 mg, 1.26 mmol), LiCl (870 mg, 20.5 mmol) and Pd(dppf)Cl₂·DCM (761 mg, 968.1 μmol) in DMF (30 mL) was degassed, back-filled with N₂ and then stirred at 120° C. for 2 h. The crude reaction mixture was filtered and the filtrate was purified by preparative HPLC C18 column eluting with ACN/water/0.1% formic acid to provide 3-amino-6-(6-fluoro-2-pyridyl)-4-(5-methyl-1H-indazol-4-yl)pyridine-2-carboxamide (2.12 g, 59% yield). MS: [M+H]⁺: 363.1.

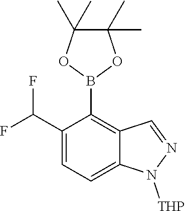

### Intermediate P (5-(difluoromethyl)-1-(tetrahydro-2H-pyran-2-yl)-4-(4,4,5,5-tetramethyl-1,3,2-dioxaborolan-2-yl)-1H-indazole)

Step 1. To a suspension of 5-bromo-4-chloro-1H-indazole (2 g, 8.64 mmol) in DCM (49 mL) at rt were added p-toluenesulfonic acid monohydrate (164 mg, 0.86 mmol) and 3,4-dihydro-2H-pyran (2.4 mL, 26.31 mmol). The mixture was stirred at rt for for 4 h. Aqueous saturated NaHCO₃ was slowly added. The layers were partitioned and the organic layer was washed with water and brine, dried over MgSO₄, filtered and concentrated to dryness. The residue was purified by silica gel chromatography eluting with a gradient of EtOAc (0 to 30%) in heptane to provide 5-bromo-4-chloro-1-(tetrahydro-2H-pyran-2-yl)-1H-indazole (2.0 g, 73% yield). ¹H-NMR (400 MHz, DMSO): δ 8.18 (s; 1H); 7.69-7.70 (m; 2H); 5.88 (dd; J=9.44; 2.35 Hz; 1H); 3.83-3.86 (m; 1H); 3.68-3.76 (m; 1H); 2.28-2.37 (m; 1H); 1.94-2.02 (m; 2H); 1.70-1.74 (m; 1H); 1.42-1.60 (m; 2H).

Step 2. A mixture of 5-bromo-4-chloro-1-(tetrahydro-2H-pyran-2-yl)-1H-indazole (3.7 g, 11.7 mmol) and vinylboronic acid pinacol ester (3 mL, 17.7 mmol) in dioxane (37 mL) and 2 M aqueous solution of Na₂CO₃ (8.8 mL, 17.6 mmol) was degassed under nitrogen atmosphere, then Pd(PPh₃)₄ (677 mg, 0.586 mmol) was added. The mixture was degassed again and heated to 100° C. for 18 h. Upon cooling to rt, the mixture was diluted with EtOAc and water and filtered through Celite™. The layers were partitioned and the organic layer was washed with brine, dried over MgSO₄, filtered and concentrated to dryness in vacuo. The residue was purified by silica gel chromatography eluting with a gradient of EtOAc (0 to 45%) in heptane to provide 4-chloro-1-(tetrahydro-2H-pyran-2-yl)-5-vinyl-1H-indazole (2.5 g, 81%). MS: [M+H]⁺: 263.1.

Step 3. To a solution of 4-chloro-1-(tetrahydro-2H-pyran-2-yl)-5-vinyl-1H-indazole (2.5 g, 9.5 mmol) in THF (50 mL) and water (12.5 mL) were added OSO₄ (4% in water, 6 mL, 0.944 mmol) and NaIO₄ (10 g, 46.7 mmol). The reaction mixture was stirred at rt for 18 h. EtOAc (120 mL) and water (120 mL) were added. The layers were partitioned and the aqueous layer was extracted with EtOAc (120 mL). The combined organic layers was washed with brine, dried over MgSO₄, filtered and concentrated to dryness. The residue was purified by silica gel chromatography eluting with a gradient of EtOAc (0 to 45%) in heptane to provide 4-chloro-1-(tetrahydro-2H-pyran-2-yl)-1H-indazole-5-carbaldehyde as a colorless oil. (1.1 g, 44% yield). MS: [M−THP+H]⁺: 181.0.

Step 4. To a solution of 4-chloro-1-(tetrahydro-2H-pyran-2-yl)-1H-indazole-5-carbaldehyde (1.1 g, 4.16 mmol) in DCM (40 mL) at rt was added XtalFluor-E (2.0 g, 8.73 mmol) followed by triethylamine trihydrofluoride (1.4 mL, 8.59 mmol). The reaction was stirred at rt for 18 h. Aqueous saturated NaHCO₃ was added dropwise to adjust to pH ˜8. The layers were partitioned and the aqueous layer was extracted with DCM. The combined organic layers was washed with brine, dried over MgSO₄, filtered and concentrated in vacuo. The residue was purified by silica gel chromatography eluting with a gradient of EtOAc (0 to 40%) in heptane to provide 4-chloro-5-(difluoromethyl)-1-(tetrahydro-2H-pyran-2-yl)-1H-indazole (0.69 g, 58% yield). MS: [M−THP+H]⁺: 203.0.

Step 5. To a solution of 4-chloro-5-(difluoromethyl)-1-(tetrahydro-2H-pyran-2-yl)-1H-indazole (250 mg, 0.872 mmol) in dioxane (3 mL) was added bis(pinacolato)diboron (443 mg, 1.75 mmol), KOAc (260 mg, 2.62 mmol), tricyclohexylphospine (61 mg, 0.218 mmol) and Pd₂(dba)₃ (40 mg, 0.044 mmol). The mixture was degassed and refilled with argon (3 cycles) and heated to 100° C. for 3 h. Upon cooling to rt, water (40 mL) and EtOAc (35 mL) were added. The layers were partitioned, the aqueous layer was extracted with EtOAc (25 mL). The combined organic layers was washed with brine, dried over MgSO₄, filtered and concentrated in vaccuo. The residue was purified by silica gel chromatography eluting with a gradient of EtOAc (0 to 40%) in heptane to provide 5-(difluoromethyl)-1-(tetrahydro-2H-pyran-2-yl)-4-(4,4,5,5-tetramethyl-1,3,2-dioxaborolan-2-yl)-1H-indazole. MS: [M+H]⁺: 379.2 [M+1]+.

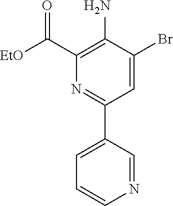 [SMILES: CCOC(=O)c1nc(-c2cccnc2)cc(Br)c1N]

Intermediate Q (ethyl 5-amino-4-bromo-[2,3′-bipyridine]-6-carboxylate) Step 1. A mixture of ethyl 3-amino-6-chloropicolinate (2.8 g, 14.0 mmol), 3-(tributylstannyl)pyridine (7.7 g, 20.9 mmol), CuI (532 mg, 2.79 mmol), LiCl (1.21 g, 27.9 mmol) and DMF (28 mL) was degassed and back-filled with nitrogen atmosphere. Pd(dppf)Cl₂·DCM (570 mg, 0.698 mmol) was added. It was degassed and back-filled with nitrogen atmosphere again. The mixture was heated to 120° C. for 18 h. Upon cooling to rt, the mixture was diluted with EtOAc and water and filtered through Celite™. The layers were partitioned and the aqueous layer was extracted again with EtOAc. The combined layers was washed with brine and dried over MgSO₄, filtered and concentrated. The residue was purified by silica gel chromatography eluting with a gradient of MeOH (0 to 6%) in EtOAc to provide ethyl 5-amino-[2,3′-bipyridine]-6-carboxylate (1.38 g, 46% yield). MS: [M+H]⁺: 244.1 [M+1]+; ¹H NMR (400 MHz, Chloroform-d) δ (s; 1H); 8.58 (d; J=4.70 Hz; 1H); 8.27-8.29 (m; 1H); 7.71 (d; J=8.67 Hz; 1H); 7.37 (dd; J=7.97; 4.78 Hz; 1H); 7.16 (d; J=8.66 Hz; 1H); 5.85 (s; 2H); 4.46 (q; J=7.13 Hz; 2H); 1.48 (t; J=7.12 Hz; 3H).

Step 2. To a suspension of ethyl 5-amino-[2,3′-bipyridine]-6-carboxylate (730 mg, 3.0 mmol) in water (20 mL) at 5° C. was added sulfuric acid (0.32 mL, 6.0 mmol). A solution of bromine (0.19 mL, 3.71 mmol) in acetic acid (2 mL, 34.94 mmol) was added dropwise and the mixture was stirred at rt for 3 h. The reaction mixture was diluted with water (40 mL) and neutralized by addition of solid NaHCO₃ (very exothermic quench). The mixture was extracted with DCM (3×30 mL). The combined organic layers was washed with saturated aqueous NaHCO₃, water and brine sequentially, dried over MgSO₄, filtered and concentrated. The residue was purified by silica gel chromatography eluting with a gradient of EtOAc (30 to 100%) in heptane to provide ethyl 5-amino-4-bromo-[2,3′-bipyridine]-6-carboxylate (0.66 g, 75% yield). MS: [M+H]⁺: 322.0/324.0.

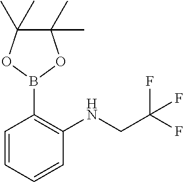 [SMILES: CC1(C)OB(c2ccccc2NCC(F)(F)F)OC1(C)C]

### Intermediate R (2-(4,4,5,5-tetramethyl-1,3,2-dioxaborolan-2-yl)-N-(2,2,2-trifluoroethyl)aniline)

Step 1. To a solution of 2-bromoaniline (5.06 g, 29.41 mmol) in Et₂O (30 mL) was added Na₂CO₃ (5.00 g, 47.17 mmol). The mixture was cooled to 0° C. and trifluoroacetic anhydride (5.39 mL, 38.3 mmol) was added dropwise. The ice-bath was removed and the reaction mixture was stirred at rt for 5.5 h. The reaction mixture was poured into water and extracted with EtOAc (3×). The combined organic layers was washed with brine, dried over Na₂SO₄, filtered and concentrated to provide N-(2-bromophenyl)-2,2,2-trifluoro-acetamide (7.67 g, 97% yield). MS: [M+H]⁺: 268.2; ¹H NMR (400 MHz, Chloroform-d) δ 8.45 (br s, 1H), 8.32 (dd, J=8.2, 1.6 Hz, 1H), 7.61 (dd, J=8.1, 1.4 Hz, 1H), 7.40 (ddd, J=8.1, 7.5, 1.5 Hz, 1H), 7.13 (ddd, J=8.2, 7.5, 1.6 Hz, 1H).

Step 2. To a solution of N-(2-bromophenyl)-2,2,2-trifluoro-acetamide (2.04 g, 7.61 mmol) in THF (5 mL) was added 1 M THF solution of borane (15 mL, 15 mmol). The mixture was stirred under reflux overnight. MeOH (5 mL) was added dropwise to the cooled reaction mixture which was then stirred at rt for 2 h and then concentrated. The residue was purified by silica gel chromatography eluting with a gradient of EtOAc (0 to 50%) in heptane to provide 2-bromo-N-(2,2,2-trifluoroethyl)aniline (463 mg, 24% yield). ¹H NMR (400 MHz, Chloroform-d) δ 7.46 (dd, J=7.9, 1.5 Hz, 1H), 7.21 (ddd, J=8.6, 7.4, 1.5 Hz, 1H), 6.80-6.73 (m, 1H), 6.68 (td, J=7.6, 1.4 Hz, 1H), 4.68 (br s, 1H), 3.83 (qd, J=8.8, 7.0 Hz, 2H).

Step 3. To a MW vial charged with 2-bromo-N-(2,2,2-trifluoroethyl)aniline (463 mg, 1.82 mmol), bis(pinacolato)diboron (555 mg, 2.19 mmol) and KOAc (533 mg, 5.43 mmol) was added dioxane (8 mL). The solution was bubbled through with N₂, and Pd(dppf)Cl₂·DCM (157 mg, 192.25 μmol) was added. The solution was bubbled through again with N₂, the vial was capped and transferred to a preheated (100° C.) heatblock for 1 h. The cooled reaction mixture was diluted with DCM and adsorbed on silica gel. The residue was purified by silica gel chromatography eluting with a gradient of EtOAc (0 to 100%) in heptane to provide 2-(4,4,5,5-tetramethyl-1,3,2-dioxaborolan-2-yl)-N-(2,2,2-trifluoroethyl)aniline (265 mg, 48% yield). MS: [M+H]⁺: 302.0.

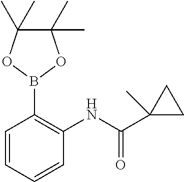 [SMILES: CC1(C(=O)Nc2ccccc2B2OC(C)(C)C(C)(C)O2)CC1]

### Intermediate S (1-methyl-N-(2-(4,4,5,5-tetramethyl-1,3,2-dioxaborolan-2-yl)phenyl)cyclopropane-1-carboxamide)

To a solution of 2-(4,4,5,5-tetramethyl-1,3,2-dioxaborolan-2-yl)aniline (10 g, 45.6 mmol), HATU (19.1 g, 50.2 mmol) and DIPEA (16.70 mL, 95.9 mmol) in DMF (100 mL) was added 1-methylcyclopropanecarboxylic acid (5.00 g, 49.94 mmol). The reaction mixture was stirred at RT for 18 h. Water (300 mL) was slowly added and the white precipitate was recover by filtration, washed with water, and dried under air flow for 5 h to provide 1-methyl-N-[2-(4,4,5,5-tetramethyl-1,3,2-dioxaborolan-2-yl)phenyl]cyclopropanecarboxamide (11.1 g, 81% yield). ¹H NMR (400 MHz, Chloroform-d) δ 9.62 (s, 1H), 8.48 (dd, J=8.5, 1.0 Hz, 1H), 7.75 (dd, J=7.4, 1.7 Hz, 1H), 7.41 (ddd, J=8.8, 7.3, 1.8 Hz, 1H), 7.03 (td, J=7.4, 1.1 Hz, 1H), 1.72 (s, 1H), 1.36 (s, 12H), 1.30 (q, J=3.9 Hz, 2H), 0.68-0.63 (m, 2H).

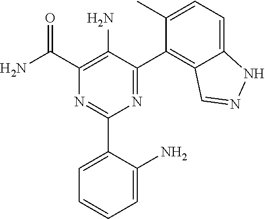 [SMILES: Cc1ccc2[nH]ncc2c1-c1nc(-c2ccccc2N)nc(C(N)=O)c1N]

### Intermediate T (5-amino-2-(2-aminophenyl)-6-(5-methyl-1H-indazol-4-yl)pyrimidine-4-carboxamide)

To a solution of Intermediate J (0.4 g, 1.32 mmol) in dioxane (6 mL) were added 2-(4,4,5,5-tetramethyl-1,3,2-dioxaborolan-2-yl)aniline (320 mg, 1.46 mmol), 2 M aqueous K₂CO₃ solution (1.9 mL, 3.8 mmol) and Pd(dppf)Cl₂ (80 mg, 109.3 μmol). The mixture was degassed in vacuo, back-filled with N₂ and stirred at 110° C. for 8 h. The mixture was diluted with water and extracted with EtOAc (3×25 mL). The combined organic extracts was washed with water and brine, dried over sodium sulfate, filtered and concentrated. The residue was purified by silica gel chromatography eluting with a gradient of EtOAc (50 to 100%) in heptane to provide 5-amino-2-(2-aminophenyl)-6-(5-methyl-1H-indazol-4-yl)pyrimidine-4-carboxamide (330 mg, 69% yield). MS: [M+H]⁺: 360.2.

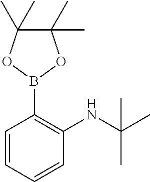 [SMILES: CC(C)(C)Nc1ccccc1B1OC(C)(C)C(C)(C)O1]

### Intermediate U (N-(tert-butyl)-2-(4,4,5,5-tetramethyl-1,3,2-dioxaborolan-2-yl)aniline)

To a MW vial charged with bis(pinacolato)diboron (336 mg, 1.32 mmol), Pd(dppf)Cl₂·DCM (110 mg, 134.7 μmol) and KOAc (356 mg, 3.63 mmol) was added 2-bromo-N-tert-butyl-aniline (263 mg, 1.15 mmol) and dioxane (4 mL). The solution was bubbled through with N₂, the vial was capped and transferred to a preheated (100° C.) heat block overnight. The cooled reaction mixture was diluted with DCM and adsorbed onto silica gel. The residue was purified by silica gel chromatography eluting with a gradient of EtOAc (0 to 100%) in heptane to provide N-tert-butyl-2-(4,4,5,5-tetramethyl-1,3,2-dioxaborolan-2-yl)aniline (135 mg, 43% yield). MS: [M+H]⁺: 276.2.

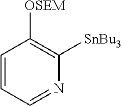

### Intermediate V (2-(tributylstannyl)-3-((2-(trimethylsilyl)ethoxy)methoxy)pyridine)

Step 1. To a solution of 2-bromopyridin-3-ol (2.5 g, 14.4 mmol) in DMF (13 mL) at 0° C. was added Et₃N (2.8 mL, 20.1 mmol), followed by 2-(trimethylsilyl)ethoxymethyl chloride (2.7 mL, 15.3 mmol). The reaction mixture was allowed to warm up slowly and stirred at rt for 4 h. The reaction mixture was diluted with water and hexanes-EtOAc (1:1). The layers were partitioned and the aqueous layer was extracted with hexanes-EtOAc (1:1). The combined organic layers were washed with brine, dried over MgSO₄, filtered and concentrated in vacuo. The residue was purified by silica gel chromatography eluting with a gradient of EtOAc (0 to 30%) in heptane to provide 2-bromo-3-((2-(trimethylsilyl)ethoxy)methoxy)pyridine (2.5 g, 57% yield). MS: [M+H]⁺: 304.1/306.1. ¹H-NMR (400 MHz, CDCl3): δ 8.04 (dd; J=4.61; 1.60 Hz; 1H); 7.45 (dd; J=8.11; 1.60 Hz; 1H); 7.20 (dd; J=8.14; 4.63 Hz; 1H); 5.32 (s; 2H); 3.80 (t; J=8.36 Hz; 2H); 0.95 (t; J=8.34 Hz; 2H); 0.00 (s; 9H).

Step 2. To a solution of n-BuLi (6.6 mL, 16.5 mmol) in THF (34 mL) at −78° C. was added dropwise a solution of 3-bromo-4-((2-(trimethylsilyl)ethoxy)methoxy)pyridine (2.5 g, 8.2 mmol) in THF (8 mL). The reaction mixture was stirred at −78° C. for 20 min. To the reaction mixture was added tributyltin chloride (2.5 mL, 9.216 mmol). The mixture was stirred at −78° C. for 40 min and at rt for 1.5 h. Water (40 mL) was added and the volatiles were removed in vacuo. The mixture was extracted with DCM (2×40 mL). The combined organic layers was washed with brine, dried over MgSO₄, filtered and concentrated. The residue was purified by silica gel chromatography eluting with a gradient of EtOAc (0 to 30%) in heptane to provide 3-(tributylstannyl)-4-((2-(trimethylsilyl)ethoxy)methoxy)pyridine (1.5 g, 35% yield). MS: [M+H]⁺: 516.2. ¹H-NMR (400 MHz, CDCl3): δ 8.41 (d; J=4.66 Hz; 1H); 7.28 (d; J=8.40 Hz; 1H); 7.07 (dd; J=8.39; 4.66 Hz; 1H); 5.20 (s; 2H); 3.73 (t; J=8.31 Hz; 2H); 1.51-1.57 (m; 8H); 1.25-1.34 (m; 8H); 1.07-1.14 (m; 8H); 0.95 (t; J=8.26 Hz; 2H); 0.87 (t; J=7.31 Hz; 12H); 0.00 (s; 9H).

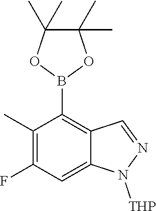

### Intermediate W (6-fluoro-5-methyl-1-(tetrahydro-2H-pyran-2-yl)-4-(4,4,5,5-tetramethyl-1,3,2-dioxaborolan-2-yl)-1H-indazole)

Step 1. 3,4-dihydro-2H-pyran (15.93 g, 189.35 mmol, 17.2 mL) was added to a solution of 4-bromo-6-fluoro-1H-indazole (20 g, 93.01 mmol) and p-toluenesulfonic acid (800 mg, 4.65 mmol) in EtOAc (100 mL). The mixture was stirred at reflux for 3 h. The volatiles were removed under vacuum. The residue was purified by silica gel chromatography eluting with a gradient of EtOAc (0 to 10%) in heptane to provide 4-bromo-6-fluoro-1-tetrahydropyran-2-yl-indazole (23.9 g, 86% yield). MS: [M+H]⁺: 299.0/301.0.

Step 2. 2 M THF/hexanes/ethylbenzene solution of lithium diisopropylamide (5.5 mL, 11.0 mmol) was added dropwise to 4-bromo-6-fluoro-1-tetrahydropyran-2-yl-indazole (1 g, 3.34 mmol) in THF (15 mL) at −78° C. under inert atmosphere. The mixture was stirred at −78° C. for 4 h. Iodomethane (750 μL, 12.05 mmol) was slowly added. The mixture was stirred for 30 minutes at −78° C. and was then allowed to warm to rt. The mixture was quenched with saturated aqueous NH₄Cl solution. The mixture was extracted with EtOAc (3×). The combined organic layers were washed with brine, dried over Na₂SO₄ filtered and concentrated. The residue was purified by silica gel chromatography eluting with a gradient of EtOAc (0 to 15%) in heptane to provide 4-bromo-6-fluoro-5-methyl-1-tetrahydropyran-2-yl-indazole (750 mg, 72% yield). MS: [M−TFP+H]⁺: 229.0/231.0.

Step 3. A flask was charged with 4-bromo-6-fluoro-5-methyl-1-tetrahydropyran-2-yl-indazole (800 mg, 2.55 mmol), KOAc (765 mg, 7.79 mmol), bis(pinacolato)diboron (750 mg, 2.95 mmol) and dioxane (10 mL). The mixture was degassed with nitrogen for 5 min and Pd(dppf)Cl₂·DCM (125 mg, 153.00 μmol) was added. The resulting mixture was degassed with nitrogen again for 2 min and was stirred 16 h at 100° C. The reaction mixture was cooled to rt, filtered through Celite™ and the filter cake was washed with ethyl acetate. The filtrate was evaporated and the crude product was purified by silica gel chromatography eluting with a gradient of EtOAc (0 to 20%) in heptane to provide 6-fluoro-5-methyl-1-tetrahydropyran-2-yl-4-(4,4,5,5-tetramethyl-1,3,2-dioxaborolan-2-yl)indazole (720 mg, 78% yield). MS: [M+H]⁺: 361.3.

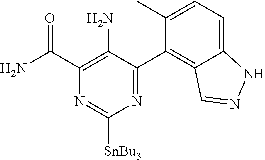

### Intermediate X (5-amino-6-(5-methyl-1H-indazol-4-yl)-2-(tributylstannyl)pyrimidine-4-carboxamide)

To a solution of Intermediate J (200 mg, 660.7 μmol) in dioxane (6 mL) were added tributyl(tributylstannyl)stannane (430 μL, 852.43 μmol), PdCl₂(dtbpf) (43 mg, 66.1 μmol). The mixture was degassed in vacuo, back-filled with N₂ and stirred at reflux for 18 h under N₂. The cooled reaction mixture was was diluted with water and extracted with EtOAc (3×15 mL). The combined organic extracts was washed with water and brine consecutively, dried over sodium sulfate, filtered and concentrated. The residue was purified by silica gel chromatography eluting with a gradient of EtOAc (50 to 100%) in heptane to provide 5-amino-6-(5-methyl-1H-indazol-4-yl)-2-tributylstannyl-pyrimidine-4-carboxamide (85 mg, 23% yield). ¹H NMR (400 MHz, Chloroform-d) δ 11.53 (s, 1H), 8.17 (d, J=4.9 Hz, 1H), 7.70 (d, J=0.9 Hz, 1H), 7.42 (dd, J=8.6, 1.0 Hz, 1H), 7.29 (d, J=8.6 Hz, 1H), 6.30 (d, J=4.9 Hz, 1H), 5.72 (s, 2H), 2.27 (s, 3H), 1.71-1.50 (m, 6H), 1.34-1.27 (m, 6H), 1.16-1.07 (m, 6H), 0.84 (t, J=7.3 Hz, 9H).

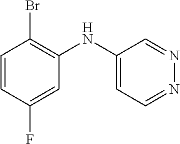 [SMILES: Fc1ccc(Br)c(Nc2ccnnc2)c1]

### Intermediate Y (N-(2-bromo-5-fluorophenyl)pyridazin-4-amine)

To a solution of 1-bromo-4-fluoro-2-iodo-benzene (570 mg, 1.89 mmol) in DMF (8 mL) were added pyridazin-4-amine (200 mg, 2.10 mmol), cesium carbonate (930 mg, 2.85 mmol), Xantphos (16 mg, 27.7 μmol) and Pd(OAc)₂ (41 mg, 182.6 μmol). The mixture was degassed in vacuo, back-filled with N₂ and stirred at 110° C. for 2 h. The volatiles were removed in vacuo and the residue was purified by silica gel chromatography eluting with a gradient of EtOAc (20 to 100%) in heptane to provide N-(2-bromo-5-fluoro-phenyl)pyridazin-4-amine (300 mg, 59% yield). MS: [M+H]⁺: 268.1.

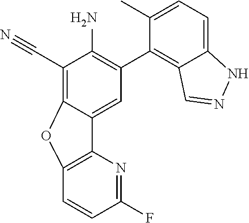 [SMILES: Cc1ccc2[nH]ncc2c1-c1cc2c(oc3ccc(F)nc32)c(C#N)c1N]

### Intermediate Z (7-amino-2-fluoro-8-(5-methyl-1H-indazol-4-yl)benzofuro[3,2-b]pyridine-6-carbonitrile)

Step 1. To a solution of 6-amino-3-bromo-2-fluorobenzonitrile (10 g, 42.0 mmol mmol) in THF (100 mL) at rt was added di-tert-butyl dicarbonate (23 g, 105.4 mmol) and DMAP (1 g, 8.19 mmol). The reaction mixture was heated to reflux for 3 h. Upon cooling to rt, the volatiles were removed in vacuo. The residue was purified by silica gel chromatography eluting with a gradient of EtOAc (10 to 30%) in hexanes to provide bis(tert-butyl) (4-bromo-2-cyano-3-fluorophenyl)carbamate (15.0 g, 86% yield). MS: [M+Na]⁺: 437.0/439.0; ¹H-NMR (400 MHz, CDCl3): δ 7.77-7.81 (m; 1H); 7.02 (d; J=8.65 Hz; 1H); 1.46 (s; 18H).

Step 2. To a solution of 2-bromo-6-fluoropyridin-3-ol (15.0 g, 36.1 mmol) in DMF (300 mL) at rt was added bis(tert-butyl) (4-bromo-2-cyano-3-fluorophenyl)carbamate (7.6 g, 39.6 mmol) and K₂CO₃ (6.0 g, 43.4 mmol). The reaction mixture was degassed (3 cycles vacuum/nitrogen atmosphere), stirred at rt for 30 min then heated to 40° C. for 18 h. Upon cooling to rt, the mixture was poured into 1:1 water/saturated aqueous NaHCO₃ solution. The mixture was extracted with EtOAc (3×300 mL). The combined organic layers was washed with water (2×), brine, dried over MgSO₄, filtered and concentrated. The residue was purified by silica gel chromatography eluting with a gradient of EtOAc (5 to 55%) in hexanes to provide 7.95 g of bis(tert-butyl) (4-bromo-3-((2-bromo-6-fluoropyridin-3-yl)oxy)-2-cyanophenyl)carbamate (7.95 g, 38% yield). MS: [M-Boc+H]⁺: 487.9; 1H-NMR (400 MHz, CDCl3): δ 7.90 (d; J=8.68 Hz; 1H); 7.16 (d; J=8.67 Hz; 1H); 6.87-6.91 (m; 1H); 6.79-6.82 (m; 1H); 1.48 (s; 18H).

Step 3. To a sealed tube was loaded bis(tert-butyl) (4-bromo-3-((2-bromopyridin-3-yl)oxy)-2-cyanophenyl)carbamate (7.95 g, 13.5 mmol), bis(pinacolato)diboron (3.78 g, 14.9 mmol), KOAc (4.0 g, 40.3 mmol), Pd(dppf)Cl₂·DCM (2.2 g, 2.7 mmol) and dioxane (80 mL). The reaction mixture was degassed (3 cycles with vacuum/nitrogen gas) and stirred at 110° C. for 8 h. Upon cooling to rt, the reaction mixture was diluted with water and EtOAc and filtered through Celite™. The layers were partitioned and the aqueous layer was extracted with EtOAc. The combined organic layers was washed with brine, dried over MgSO₄, filtered and concentrated. The residue was purified by silica gel chromatography eluting with a gradient of EtOAc (5 to 35%) in hexanes to provide bis(tert-butyl) (6-cyano-2-fluorobenzofuro[3,2-b]yridine-7-yl)carbamate (3.2 g, 55% yield). MS: [M-Boc+H]⁺: 328.1; 1H-NMR (400 MHz, CDCl3): δ 8.34 (d; J=8.34 Hz; 1H); 8.09 (t; J=7.53 Hz; 1H); 7.37 (d; J=8.38 Hz; 1H); 7.15 (d; J=8.88 Hz; 1H); 1.46 (s; 18H).

Step 4. A solution of bis(tert-butyl) (6-cyano-2-fluorobenzofuro[3,2-b]yridine-7-yl)carbamate (2.9 g, 6.8 mmol) in acetic acid (58 mL) was heated to reflux for 35 h. The solvent was removed under vacuum, co-evaporated with n-heptane (3×) and dried under high vacuum to afford 1.6 g of 7-amino-2-fluorobenzofuro[3,2-b]pyridine-6-carbonitrile which was used as such in the subsequent step without further purification. MS: [M+H]⁺: 228.0.

Step 5. To a solution of 7-amino-2-fluorobenzofuro[3,2-b]pyridine-6-carbonitrile (1.9 g, 8.4 mmol) in DMF (20 mL) at rt was added NBS (1.5 g, 8.4 mmol). The reaction mixture was allowed to stir at rt for 1 h then diluted with 20% wt aqueous solution of Na₂S₂O₃ (80 mL). The mixture was gently stirred at rt and the solids were collected by filtration. The filter cake was washed with water and (1:1) n-heptane-MTBE (30 mL). The solids were dried under high vacuum to afford 7-amino-8-bromo-2-fluorobenzofuro[3,2-b]pyridine-6-carbonitrile (2.3 g, 89% yield). MS: [M+H]⁺: 305.9/307.9; ¹H NMR (400 MHz, DMSO-d6) δ 8.31-8.37 (m; 1H); 7.18 (d; J=8.70 Hz; 1H); 7.01 (s; 1H).

Step 6. A mixture of 7-amino-8-bromo-2-fluorobenzofuro[3,2-b]pyridine-6-carboxamide (1.9 g, 3.2 mmol), (5-methyl-1H-indazol-4-yl)boronic acid (2.2 g, 12.5 mmol), 2 M aqueous solution of K₃PO₄ (9.3 mL, 18.6 mmol), Pd₂(dba)₃ (570 mg, 0.622 mmol), tri-tert-butylphosphonium tetrafluoroborate (360 mg, 1.241 mmol) and dioxane (38 mL) was degassed (3 cycles of vacuum/nitrogen atmosphere). The mixture was heated to reflux for 2 h, cooled to rt, poured into a mixture of water (50 mL) and EtOAc (70 mL), and filtered through Celite™. The layers were partitioned and the aqueous layer was extracted with EtOAc (70 mL). The combined organic layers was washed with water and brine, dried over MgSO₄, filtered and concentrated. The residue was purified by silica gel chromatography eluting with a gradient of EtOAc (30 to 100%) in hexanes to provide 7-amino-2-fluoro-8-(5-methyl-1H-indazol-4-yl)benzofuro[3,2-b]pyridine-6-carbonitrile (1.2 g, 57% yield). MS: [M+H]⁺: 358.1.

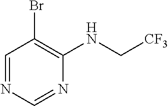 [SMILES: FC(F)(F)CNc1ncncc1Br]

### Intermediate AA (5-bromo-N-(2,2,2-trifluoroethyl)pyrimidin-4-amine)

To a solution of 1-bromo-4-fluoro-2-iodo-benzene (570 mg, 1.89 mmol) in DMF (8 mL) were added pyridazin-4-amine (200 mg, 2.10 mmol), cesium carbonate (930 mg, 2.85 mmol), Xantphos (16 mg, 27.7 μmol) and Pd(OAc)₂ (41 mg, 182.6 μmol). The mixture was degassed in vacuo, back-filled with N₂ and stirred at 110° C. for 2 h. The volatiles were removed in vacuo and the residue was purified by silica gel chromatography eluting with a gradient of EtOAc (20 to 100%) in heptane to provide N-(2-bromo-5-fluoro-phenyl)pyridazin-4-amine (300 mg, 59% yield). MS: [M+H]⁺: 268.1.

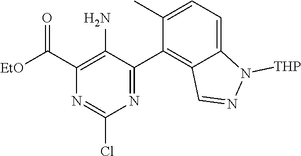

### Intermediate AB (ethyl 5-amino-2-chloro-6-(5-methyl-1-(tetrahydro-2H-pyran-2-yl)-1H-indazol-4-yl)pyrimidine-4-carboxylate)

A mixture of ethyl 5-amino-2,6-dichloro-pyrimidine-4-carboxylate (10 g, 42.36 mmol), 5-methyl-1-tetrahydropyran-2-yl-4-(4,4,5,5-tetramethyl-1,3,2-dioxaborolan-2-yl)indazole (16 g, 46.75 mmol), Pd(dtbpf)Cl₂ (2.75 g, 4.22 mmol) and 2 M aqueous solution of K₃PO₄ (34 mL, 68 mmol) in dioxane (200 mL) was bubbled through with N₂, then heated at 80° C. for 5 h. The cooled reaction mixture was poured into water and extracted with EtOAc (3×). The combined organic layers was washed with brine, dried over Na₂SO₄, filtered and concentrated. The residue was purified by silica gel chromatography eluting with a gradient of EtOAc (0 to 70%) in heptane to provide ethyl 5-amino-2-chloro-6-(5-methyl-1-tetrahydropyran-2-yl-indazol-4-yl)pyrimidine-4-carboxylate (13.1 g, 74% yield). MS: [M+H]⁺: 416.3.

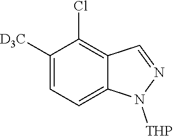

### Intermediate AC (4-chloro-5-(methyl-d3)-1-(tetrahydro-2H-pyran-2-yl)-1H-indazole)

Step 1. To a suspension of 5-bromo-4-chloro-1H-indazole (6.8 g, 29.4 mmol) in DCM (170 mL) at rt were added p-toluenesulfonic acid monohydrate (559 mg, 2.9 mmol) and 3,4-dihydro-2H-pyran (8 mL, 87.7 mmol). The mixture was stirred at rt for 18 h. Aqueous saturated NaHCO₃ was slowly added. The layers were partitioned and the organic layer was washed with water and brine, dried over MgSO₄, filtered and concentrated. The residue was purified by silica gel chromatography eluting with a gradient of EtOAc (0 to 30%) in hexanes to provide 4-bromo-5-methyl-1-(tetrahydro-2H-pyran-2-yl)-1H-indazole (9.0 g, 97% yield). ¹H-NMR (400 MHz, DMSO): δ 8.18 (s; 1H); 7.69-7.70 (m; 2H); 5.88 (dd; J=9.44; 2.35 Hz; 1H); 3.83-3.86 (m; 1H); 3.68-3.76 (m; 1H); 2.28-2.37 (m; 1H); 1.94-2.02 (m; 2H); 1.70-1.74 (m; 1H); 1.42-1.60 (m; 2H).

Step 2. A round bottom flask (250 mL) equipped with a condenser was flame-dried and charged with magnesium (1.24 g, 51.0 mmol). The flask was flame-dried again and iodine (104 mg, 408 μmol) was added. The flask was degassed (3 cycles of vacuum/argon atmosphere) then degassed Et₂O (62 mL) was added followed by dropwise addition of Iodomethane-d3 (3.18 mL, 51.0 mmol). After adding a few drops of iodomethane-d3, the mixture was sonicated for 5 min. The color of the reaction changed from orange to yellow, then milky and finally cloudy metallic (high exotherm observed, the suspension was refluxed without external heat). After the addition of iodomethane-d3, the reaction mixture became more metallic. It was stirred at rt for 1.5 h.

A clear solution of zinc chloride (0.5 M in THF, 51.0 mL, 25.5 mmol) was loaded into a flame-dried 3-neck flask. To this was added dropwise the Grignard suspension via a syringe (exotherm observed) and the mixture became milky. After the addition of the Grignard reagent, a white precipitate formation was observed. The reaction mixture was stirred at rt for 40 min before being used in the next step. The supernatant was used directly for next step.

Step 3. A 3-neck flask (250 mL) equipped with a condenser was flame-dried. 4-Bromo-5-methyl-1-(tetrahydro-2H-pyran-2-yl)-1H-indazole (4.00 g, 12.7 mmol), tri-tert-butylphosphonium tetrafluoroborate (735 mg, 2.5 mmol) and Pd₂(dba)₃ (1.16 g, 1.27 mmol) were loaded into the flask. The mixture was degassed (3 cycles of vacuum/argon atmosphere). The flask was covered from light and a solution of dimethyl-d6 zinc made in the previous step (70 mL, 25.5 mmol) was added dropwise via a syringe. After the addition, the mixture was heated to 70° C. for 2 h. Upon cooling to rt, the mixture was diluted with aqueous saturated NH₄Cl (100 mL) and EtOAc (150 mL) and filtered through Celite™. The layers were partitioned and the aqueous layer was extracted with EtOAc (50 mL). The combined organic layers was washed with brine, dried over MgSO₄, filtered and concentrated. The residue was purified by silica gel chromatography eluting with a gradient of 15% EtOAc-hexanes (0 to 70%) in hexanes to provide 4-chloro-5-(methyl-d3)-1-(tetrahydro-2H-pyran-2-yl)-1H-indazole (2.9 g, 90% yield). MS: [M+H]⁺: 254.1; ¹H-NMR (400 MHz, CDCl3): δ 8.04 (s; 1H); 7.40 (d; J=8.51 Hz; 1H); 7.25 (d, J=8.50 Hz, 1H), 5.67-5.70 (m; 1H); 3.98-4.03 (m; 1H); 3.70-3.75 (m; 1H); 2.50-2.58 (m; 1H); 2.14-2.16 (m; 1H); 2.07 (dd; J=14.39; 4.34 Hz; 1H); 1.67-1.76 (m; 3H).

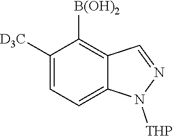

### Intermediate AD ((5-(methyl-d3)-1-(tetrahydro-2H-pyran-2-yl)-1H-indazol-4-yl)boronic acid)

Intermediate AC (5.00 g, 19.7 mmol), tetrahydroxydiboron (5.30 g, 59.1 mmol), potassium acetate (5.80 g, 59.1 mmol) and chloro(2-dicyclohexylphosphino-2′,4′,6′-triisopropyl-1,1′-biphenyl)[2-(2′-amino-1,1′-biphenyl)]palladium(II) (774 mg, 985 μmol) were loaded into a RBF equipped with a condenser. The mixture was degassed (3 cycles of vacuum/argon gas). MeOH (60 mL) and ethylene glycol (20 mL) were added and the mixture was stirred at 60° C. for 3 h. The mixture was filtered through Celite™ and the filter cake was washed with MeOH. The volatiles were removed in vacuo and the residue was purified by silica gel chromatography eluting with a gradient of EtOAc (0 to 60%) in hexanes to provide (5-(methyl-d3)-1-(tetrahydro-2H-pyran-2-yl)-1H-indazol-4-yl)boronic acid acid (3.26 g, 63% yield). MS: [M+H]⁺: 264.1.

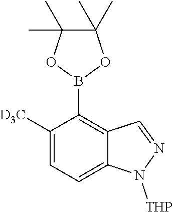

### Intermediate AE (5-(methyl-d3)-1-(tetrahydro-2H-pyran-2-yl)-4-(4,4,5,5-tetramethyl-1,3,2-dioxaborolan-2-yl)-1H-indazole)

A RBF was charged with 4-chloro-1-tetrahydropyran-2-yl-5-(trideuteriomethyl)indazole (7.15 g, 28.2 mmol), XPhos (0.675 g, 1.42 mmol), bis(pinacolato)diboron (8.60 g, 33.86 mmol) and potassium 2-ethylhexanoate (11.70 g, 64.2 mmol). IPAc (145 mL) was added, the vessel was capped and the air was replaced with N₂ (3 cycles of vacuum/N₂). The mixture was stirred at 50° C. for 5 min then XPhos Pd(allyl)Cl (0.925 g, 1.40 mmol) was added. The mixture was stirred under N₂ at 50° C. for 18 h. The reaction mixture was cooled to rt, diluted with 100 mL of EtOAc and 50 mL of water. The aqueous layer was cut and the organic layer was washed with brine, dried over MgSO₄, filtered and concentrated. The residue was purified by silica gel chromatography eluting with a gradient of EtOAc (20 to 100%) in heptane to provide 1-tetrahydropyran-2-yl-4-(4,4,5,5-tetramethyl-1,3,2-dioxaborolan-2-yl)-5-(trideuteriomethyl)indazole (9.54 g, 98% yield). MS: [M+H]⁺: 346.3.

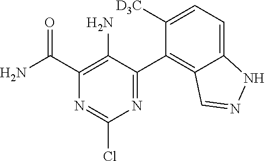 [SMILES: [2H]C([2H])([2H])c1ccc2[nH]ncc2c1-c1nc(Cl)nc(C(N)=O)c1N]

### Intermediate AF (5-amino-2-chloro-6-(5-(methyl-d3)-1H-indazol-4-yl)pyrimidine-4-carboxamide)

Step 1. Ethyl 5-amino-2,6-dichloropyrimidine-4-carboxylate (300 mg, 1.27 mmol), (5-(methyl-d3)-1-(tetrahydro-2H-pyran-2-yl)-1H-indazol-4-yl)boronic acid (368 mg, 1.4 mmol), Pd(dtbpf)Cl₂ (84.1 mg, 0.127 mmol), 2 M aqueous solution of K₃PO₄ (1.02 mL, 2.04 mmol) and dioxane (4.20 mL) were placed in a round bottom flask. The mixture was bubbled with nitrogen gas for 10 min and heated to 80° C. for 5 h under nitrogen. The cooled reaction mixture was concentrated and the residue was purified by silica gel chromatography eluting with a gradient of EtOAc (10 to 60%) in hexanes to provide 5-amino-2-chloro-6-(5-(methyl-d3)-1H-indazol-4-yl)pyrimidine-4-carboxamide (310 mg, 58% yield). MS: [M+H]⁺: 419.1; ¹H-NMR (400 MHz, CDCl₃): δ 7.61-7.71 (m; 2H); 7.36 (dd; J=8.64; 5.91 Hz; 1H); 5.66-5.76 (m; 3H); 4.51 (q; J=7.11 Hz; 2H); 3.96-4.09 (m; 1H); 3.71-3.80 (m; 1H); 2.47-2.58 (m; 1H); 2.08-2.16 (m; 2H); 1.66-1.78 (m; 3H); 1.48 (t; J=7.11 Hz; 3H).

Step 2. Ethyl 5-amino-2-chloro-6-(5-(methyl-d3)-1-(tetrahydro-2H-pyran-2-yl)-1H-indazol-4-yl)pyrimidine-4-carboxylate (270 mg, 0.645 mmol) was placed in microwave reaction vial and ammonia solution (7 N in MeOH, 9.21 mL, 64.5 mmol) was added. The reaction mixture was heated to 80° C. for 1.5 h. Upon cooling to rt, the volatiles were removed in vacuo and put under high vacuum to afford 5-amino-2-chloro-6-(5-(methyl-d3)-1-(tetrahydro-2H-pyran-2-yl)-1H-indazol-4-yl)pyrimidine-4-carboxamide (250 mg, 99% yield). MS: [M+H]⁺: 390.1.

Step 3. To a solution of 5-amino-2-chloro-6-(5-(methyl-d3)-1-(tetrahydro-2H-pyran-2-yl)-1H-indazol-4-yl)pyrimidine-4-carboxamide (130 mg, 0.33 mmol) in MeOH (1.7 mL) was added HCl (4 M in dioxane, 4.2 mL, 16.8 mmol). The reaction mixture was stirred at for 18 h. The volatiles were removed under vacuum to afford 5-amino-2-chloro-6-(5-(methyl-d3)-1H-indazol-4-yl)pyrimidine-4-carboxamide (100 mg, 99% yield). MS: [M+H]⁺: 306.0. ¹H-NMR (400 MHz, DMSO): δ 7.80 (s, 1H); 7.70 (s, 2H); 7.60 (s, 1H); 7.50 (d, J=8.4 Hz, 1H); 7.41 (s, 1H); 7.26 (d, J=8.4 Hz, 1H).

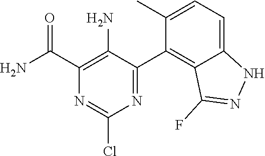 [SMILES: Cc1ccc2[nH]nc(F)c2c1-c1nc(Cl)nc(C(N)=O)c1N]

### Intermediate AF (5-amino-2-chloro-6-(3-fluoro-5-methyl-1H-indazol-4-yl)pyrimidine-4-carboxamide)

A solution of SelectFluor (1.76 g, 4.97 mmol) in DMF (5 mL) was added to a solution of Intermediate J (500 mg, 1.65 mmol) in DMF (5 mL) at 50° C. over 10 h using syringe pump. The reaction mixture was stirred at 50° C. for 10 h then diluted with water and was extracted with EtOAc (3×). The combined organic layers was washed with brine, dried over MgSO₄, filtered and concentrated to dryness. The residue was purified by preparative HPLC to provide 5-amino-2-chloro-6-(3-fluoro-5-methyl-1H-indazol-4-yl)pyrimidine-4-carboxamide (20 mg, 4% yield).

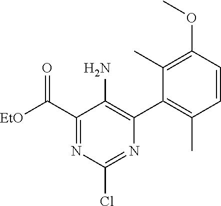 [SMILES: CCOC(=O)c1nc(Cl)nc(-c2c(C)ccc(OC)c2C)c1N]

### Intermediate AG (ethyl 5-amino-2-chloro-6-(3-methoxy-2,6-dimethylphenyl)pyrimidine-4-carboxylate)

A flame-dried MW vessel was charged with Pd₂(dba)₃ (388 mg, 0.424 mmol), tri-tert-butylphosphonium tetrafluoroborate (246 mg, 0.847 mmol), 2-(3-methoxy-2,6-dimethylphenyl)-4,4,5,5-tetramethyl-1,3,2-dioxaborolane (1.67 g, 6.4 mmol) and ethyl 5-amino-2,6-dichloropyrimidine-4-carboxylate (1.0 g, 4.2 mmol). Dioxane (8 mL) and water (2 mL) were added followed by K₃PO₄ (1.8 g, 8.5 mmol). The mixture was degassed, the vessel was sealed and the mixture was stirred at 100° C. for 10 h. The cooled reaction mixture was diluted with EtOAc and filtered through Celite™. The filterate was concentrated to dryness and diluted with water and extracted with EtOAc (3×). The combined organic layers was washed with brine, dried over MgSO₄, filtered and concentrated in vacuo. The residue was purified by silica gel chromatography eluting with a gradient of EtOAc (0 to 20%) in hexanes to provide ethyl 5-amino-2-chloro-6-(3-methoxy-2,6-dimethylphenyl)pyrimidine-4-carboxylate (330 mg, 23% yield). MS: [M+H]⁺: 336.1. ¹H NMR (CDCl3, 400 MHz): δH 1.45 (3H, t, J=7.1 Hz), 1.92 (3H, s), 1.98 (3H, s), 3.82 (3H, s), 4.48 (2H, q, J=7.1 Hz), 5.62 (2H, s), 6.85 (1H, d, J=8.4 Hz), 7.10 (1H, d, J=8.4 Hz).

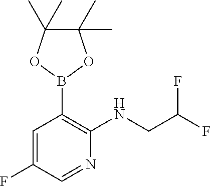 [SMILES: CC1(C)OB(c2cc(F)cnc2NCC(F)F)OC1(C)C]

### Intermediate AH (N-(2,2-difluoroethyl)-5-fluoro-3-(4,4,5,5-tetramethyl-1,3,2-dioxaborolan-2-yl)pyridin-2-amine)

Step 1. To a MW vial containing 3-bromo-2,5-difluoro-pyridine (622 mg, 3.21 mmol) and 2,2-difluoroethanamine (321 mg, 3.96 mmol) was added DMF (6 mL) followed by K₂CO₃ (884 mg, 6.40 mmol). The vial was capped and transferred to preheated (120° C.) heat block and stirred overnight. More 2,2-difluoroethanamine (1.27 g, 15.60 mmol, 1.1 mL) was added and the mixture was stirred overnight at 120° C. More 2,2-difluoroethanamine (1.27 g, 15.60 mmol, 1.1 mL) was added and the mixture was stirred at 120° C. for 3 more days. The cooled reaction mixture was poured into water and extracted with EtOAc (3×). The combined organic layers were washed with aqueous saturated NH₄Cl, brine, dried over Na₂SO₄, filtered and concentrated. The residue was purified by silica gel chromatography eluting with a gradient of EtOAc (0 to 30%) in heptane to provide 3-bromo-N-(2,2-difluoroethyl)-5-fluoro-pyridin-2-amine (333 mg, 41% yield). MS: [M+H]⁺: 255.0/257.0.

Step 2. To a MW vial charged with bis(pinacolato)diboron (403 mg, 1.59 mmol), 3-bromo-N-(2,2-difluoroethyl)-5-fluoro-pyridin-2-amine (333 mg, 1.31 mmol), KOAc (388 mg, 3.95 mmol) and Pd(dppf)Cl₂·DCM (114 mg, 139.6 μmol) was added dioxane (6 mL). The solution was bubbled through with N₂, the vial was capped and transferred to a preheated (100° C.) heat block for 2.5 h. The cooled reaction mixture was adsorbed on silica and the residue was purified by silica gel chromatography eluting with a gradient of EtOAc (0 to 50%) in heptane to provide N-(2,2-difluoroethyl)-5-fluoro-3-(4,4,5,5-tetramethyl-1,3,2-dioxaborolan-2-yl)pyridin-2-amine (301 mg, 76% yield). MS: [M+H]⁺: 303.2.

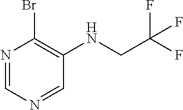 [SMILES: FC(F)(F)CNc1cncnc1Br]

### Intermediate AI (4-bromo-N-(2,2,2-trifluoroethyl)pyrimidin-5-amine)

A flame dried MW tube was loaded with THF (2 mL) and 4-amino-5-bromopyrimidine (200 mg, 1.15 mmol). The reaction mixture was heated to 80° C., phenylsilane (355 μL, 2.87 mmol) was added followed by TFA (106 μL, 1.38 mmol). The reaction was kept at 80° C. for 6 h. Upon cooling to rt, the volatiles were removed in vacuo. The residue was dissolved in EtOAc and washed with saturated aqueous sodium bicarbonate. The organic layer was dried over MgSO₄, filtered and concentrated. The residue was purified by silica gel chromatography eluting with a gradient of EtOAc (0 to 90%) in hexanes to provide 4-bromo-N-(2,2,2-trifluoroethyl)pyrimidin-5-amine (230 mg, 56% yield). MS: [M+H]⁺: 255.8.

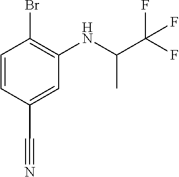 [SMILES: CC(Nc1cc(C#N)ccc1Br)C(F)(F)F]

### Intermediate AJ (4-bromo-N-(2,2,2-trifluoroethyl)pyrimidin-5-amine)

To a solution of 3-amino-4-bromobenzonitrile (269 μL, 1.02 mmol) and 1,1,1-trifluoroacetone (145 μL, 1.62 mmol) in DCM (3 mL) was added TFA (3 mL) followed by sodium cyanoborohydride (128 mg, 2.03 mmol). The reaction mixture was stirred at rt for 18 h and then concentrated to dryness. The residue was purified by silica gel chromatography eluting with a gradient of EtOAc (0 to 90%) in hexanes to provide 4-bromo-3-((1,1,1-trifluoropropan-2-yl)amino)benzonitrile (190 mg, 64% yield). MS: [M+H]⁺: 294.9.

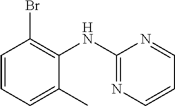

### Intermediate AK (N-(2-bromo-6-methylphenyl)pyrimidin-2-amine)

To a MW vial charged with 2-bromo-6-methyl-aniline (301 mg, 1.62 mmol) and 2-fluoropyrimidine (198 mg, 2.02 mmol) was added trifluoroethanol (3 mL) and TFA (374 μL, 4.85 mmol). The vial was capped and submitted to microwave irradiation for 60 min at 80° C. More 2-fluoropyrimidine (138 mg, 1.41 mmol) was added. The vial was capped and submitted to microwave irradiation for 20 min at 80° C. The cooled reaction mixture was concentrated. The residue was dissolved in DCM and washed with saturated aqueous solution of NaHCO₃. The aqueous layer was extracted with DCM (3×) and the combined organic layers was dried over Na₂SO₄, filtered and concentrated. The residue was purified by silica gel chromatography eluting with a gradient of EtOAc (0 to 60%) in heptane to provide N-(2-bromo-6-methyl-phenyl)pyrimidin-2-amine (230 mg, 54% yield). MS: [M+H]⁺: 264.0.

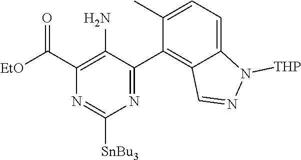

### Intermediate AL (ethyl 5-amino-6-(5-methyl-1-(tetrahydro-2H-pyran-2-yl)-1H-indazol-4-yl)-2-(tributylstannyl)pyrimidine-4-carboxylate)

A MW vessel was charged with Intermediate AB (500 mg, 1.20 mmol), dioxane (7 mL), bis(tributyltin) (1.2 mL, 2.38 mmol) and bis(di-tert-butyl(4-dimethylaminophenyl)phosphine) dichloropalladium(II) (85.1 mg, 120.2 μmol). The vessel was flushed with N₂, sealed and stirred at 110° C. overnight. The volatiles were removed in vacuo and the residue was purified by silica gel chromatography eluting with a gradient of EtOAc (0 to 40%) in heptane to provide ethyl 5-amino-6-(5-methyl-1-(tetrahydro-2H-pyran-2-yl)-1H-indazol-4-yl)-2-(tributylstannyl)pyrimidine-4-carboxylate (464 mg, 58% yield). MS: [M+H]⁺: 670.3.

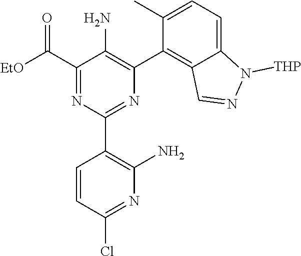

### Intermediate AM (ethyl 5-amino-2-(2-amino-6-chloropyridin-3-yl)-6-(5-methyl-1-(tetrahydro-2H-pyran-2-yl)-1H-indazol-4-yl)pyrimidine-4-carboxylate)

To a solution of Intermediate AL (4.2 g, 6.26 mmol) in DMF (50 mL) were added 6-chloro-3-iodo-pyridin-2-amine (2 g, 7.86 mmol), CuI (242 mg, 1.27 mmol), bis(tri-tert-butylphosphine)palladium(0) (350 mg, 684.9 μmol) and LiCl (570 mg, 13.5 mmol). The mixture was degassed in vacuo, back-filled with N₂ and stirred at 110° C. for 1 h under nitrogen. The volatiles were removed in vacuo and the residue was purified by silica gel chromatography eluting with a gradient of EtOAc (20 to 80%) in heptane to provide ethyl 5-amino-2-(2-amino-6-chloro-3-pyridyl)-6-(5-methyl-1-tetrahydropyran-2-yl-indazol-4-yl)pyrimidine-4-carboxylate (1.62 g, 51% yield). MS: [M+H]⁺: 508.3.

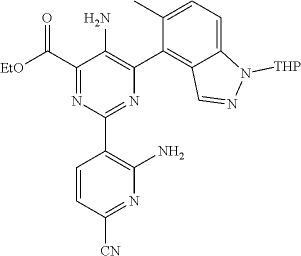

### Intermediate AN (ethyl 5-amino-2-(2-amino-6-cyanopyridin-3-yl)-6-(5-methyl-1-(tetrahydro-2H-pyran-2-yl)-1H-indazol-4-yl)pyrimidine-4-carboxylate)

To a solution of Intermediate AM (340 mg, 669.3 μmol) in DMF (5 mL) were added zinc cyanide (170 mg, 1.45 mmol) and bis(tri-tert-butylphosphine)palladium(0) (150 mg, 129.8 μmol). The mixture was degassed in vacuo, back-filled with N₂ and stirred at 120° C. for 18 h under N₂. The volatiles were removed in vacuo. The residue was purified by silica gel chromatography eluting with a gradient of EtOAc (40 to 100%) in heptane to provide ethyl 5-amino-2-(2-amino-6-cyano-3-pyridyl)-6-(5-methyl-1-tetrahydropyran-2-yl-indazol-4-yl)pyrimidine-4-carboxylate (248 mg, 74% yield). MS: [M+H]⁺: 499.3.

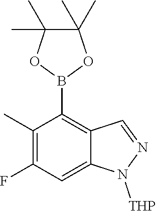

### Intermediate AO (6-fluoro-5-methyl-1-(tetrahydro-2H-pyran-2-yl)-4-(4,4,5,5-tetramethyl-1,3,2-dioxaborolan-2-yl)-1H-indazole)

Step 1. To a solution of 1,5-difluoro-2-methyl-4-nitro-benzene (25.14 g, 145.2 mmol) in Reagent alcohol (150 mL) and water (150 mL) was added concentrated hydrochloric acid, 36% w/w aqueous solution (12.5 mL). The mixture was heated at 80° C. and iron powder (28.6 g, 512.1 mmol) was added slowly in portions over a period of 35 minutes. The mixture was stirred at the same temperature for 30 min. The cooled reaction mixture was basified to approx pH 8 with saturated aqueous NaHCO₃ and diluted with EtOAc (300 mL). The layers were filtered and the solids were washed with portions of EtOAc and water. Layers from the filtrate were separated and the aqueous layer was extracted with EtOAc (2×). The combined organic layers was washed with brine, dried over Na₂SO₄, filtered over a silica pad, washed with EtOAc and concentrated under reduced pressure to afford 2,4-difluoro-5-methyl-aniline (19.98 g, 96% yield). MS: [M+H]⁺: 144.1.

Step 2. To a solution of 2,4-difluoro-5-methyl-aniline (19.42 g, 135.7 mmol) in DCM (250 mL) at 0° C. was added NBS (24.8 g, 139.3 mmol) in portions, over 5-10 min. The reaction mixture was stirred at 0° C. for 5 min then the ice bath was removed. The reaction was stirred for 15 min and then concentrated. The residue was purified by silica gel chromatography eluting with a gradient of EtOAc (0 to 25%) in heptane to provide 2-bromo-4,6-difluoro-3-methyl-aniline (18.1 g, 60% yield). MS: [M+H]⁺: 221.9.

Step 3. A RBF was loaded with ice-cold water (80 mL) and sulfuric acid (18 M, 70 mL) and was cooled in an ice bath. 2-bromo-4,6-difluoro-3-methyl-aniline (16.44 g, 74.04 mmol) in ACN (120 mL) was added dropwise via an addition funnel. The resulting suspension was stirred for 15 min at 0° C. Sodium nitrite (10.22 g, 148.1 mmol) in water (80 mL) was added dropwise. After stirring for 30 min, a solution of potassium iodide (49.17 g, 296.2 mmol) in water (130 mL) was added dropwise via an addition funnel. After the addition, the ice bath was removed and the mixture was stirred overnight. The mixture was placed in an ice bath and quenched with slow addition (addition funnel) of 20% Na₂S₂O₃ aqueous solution (250 mL). EtOAc (250 mL) was added to the mixture and the layers were separated. The aqueous layer was extracted with EtOAc. The combined organic extracts was washed with 20% Na₂S₂O₃ solution (2×250 mL), water and brine, dried over Na₂SO₄, filtered and concentrated. The residue was purified by silica gel chromatography eluting with heptane to provide 3-bromo-1,5-difluoro-2-iodo-4-methyl-benzene (20.19 g, 82% yield).

Step 4. To a solution of 3-bromo-1,5-difluoro-2-iodo-4-methyl-benzene (20.5 g, 61.58 mmol) in THF (120 mL) at −78° C. was added n-butyllithium solution 2.5 M in hexanes (27 mL, 67.5 mmol) dropwise. The reaction mixture was stirred at −78° C. for 35 min. Dry DMF (6.0 mL, 77.49 mmol) was added dropwise and the mixture was stirred at −78° C. for 1 h. The reaction mixture was quenched with 1 N aqueous HCl solution (150 mL). Water was added to the mixture and the aqueous layer was extracted with EtOAc (3×). The combined organic layers was washed with water and brine consecutively, dried over Na₂SO₄, filtered and concentrated. The residue was purified by silica gel chromatography eluting with a gradient of EtOAc (0 to 20%) in heptane to provide 2-bromo-4,6-difluoro-3-methyl-benzaldehyde (8.09 g, 56% yield). ¹H NMR (400 MHz, Chloroform-d) δ 10.33 (d, J=1.5 Hz, 1H), 6.90 (dd, J=10.3, 9.3 Hz, 1H), 2.36 (dd, J=2.6, 1.1 Hz, 3H).

Step 5. To a mixture of 2-bromo-4,6-difluoro-3-methyl-benzaldehyde (8.09 g, 34.42 mmol) in DMSO (45 mL) was added hydrazine hydrate (20 mL, 411.5 mmol). The mixture was stirred at 130° C. under open atmosphere for 1 h. The mixture was cooled down to rt and water (100 mL) was added dropwise under stirring until a white precipitate was observed. After stirring for an additional 30 min the suspension was filtered and the solids were washed with portions of water, air-dried then dried in vacuo to yield 4-bromo-6-fluoro-5-methyl-1H-indazole (6.26 g, 79% yield). MS: [M+H]+: 230.9

Step 6. 3,4-dihydro-2H-pyran (5.07 mL, 55.8 mmol) was added to a solution of 4-bromo-6-fluoro-5-methyl-1H-indazole (6.26 g, 27.3 mmol) and p-toluenesulfonic acid (239 mg, 1.39 mmol) in EtOAc (100 mL). The mixture was heated at reflux for 2.5 h. The cooled reaction mixture was diluted with saturated aqueous NaHCO₃ solution and the layers were separated. The aqueous layer was extracted with EtOAc (2×). The combined organic layers were washed with brine, dried over Na₂SO₄, filtered and concentrated in vacuo. The residue was purified by silica gel chromatography eluting with a gradient of EtOAc (0 to 30%) in heptane to provide 4-bromo-6-fluoro-5-methyl-1-tetrahydropyran-2-yl-indazole (6.51 g, 76% yield). MS: [M+H]+: 314.9; ¹H NMR (400 MHz, Chloroform-d) δ 7.95 (d, J=0.9 Hz, 1H), 7.25-7.20 (m, 1H), 5.61 (dd, J=9.1, 2.8 Hz, 1H), 3.99 (dtd, J=11.4, 3.6, 1.5 Hz, 1H), 3.76-3.68 (m, 1H), 2.54-2.43 (m, 1H), 2.41 (d, J=2.6 Hz, 3H), 2.19-2.02 (m, 2H), 1.82-1.62 (m, 3H).

Step 7. A RBF was charged with 4-bromo-6-fluoro-5-methyl-1-tetrahydropyran-2-yl-indazole (7.4 g, 23.6 mmol), potassium acetate (7.1 g, 72.4 mmol), bis(pinacolato)diboron (6.90 g, 27.2 mmol) and dioxane (150 mL). The mixture was degassed with nitrogen for 5 min and to the reaction mixture was added Pd(dppf)Cl₂·DCM (1.2 g, 1.47 mmol). The resulting mixture was degassed with nitrogen again for 2 min and was stirred at 100° C. for 16 h. The reaction mixture was cooled to rt, filtered through Celite™ and the Celite™ cake was washed with ethyl acetate. The filtrate was evaporated and the residue was purified by silica gel chromatography eluting with a gradient of EtOAc (0 to 20%) in heptane to provide 6-fluoro-5-methyl-1-tetrahydropyran-2-yl-4-(4,4,5,5-tetramethyl-1,3,2-dioxaborolan-2-yl)indazole (5.1 g, 60% yield). MS: [M+H]+: 361.3.

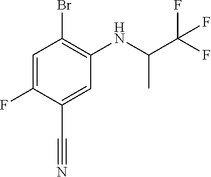 [SMILES: CC(Nc1cc(C#N)c(F)cc1Br)C(F)(F)F]

### Intermediate AP (4-bromo-N-(2,2,2-trifluoroethyl)pyrimidin-5-amine)

To a solution of 5-amino-4-bromo-2-fluorobenzonitrile (269 μL, 2.33 mmol) in DCM (4 mL) at 0° C. was added sodium cyanoborohydride (292 mg, 4.65 mmol) followed by a slow addition of TFA (4.00 mL). The resulting foaming mixture was stirred at 0° C. 1,1,1-Trifluoroacetone (333 μL, 3.72 mmol) was added slowly and the mixture was allowed to stir at rt overnight. The reaction mixture was concentrated, diluted carefully with aqueous saturated NaHCO₃ to basic pH and extracted with EtOAc (3×). The combined organic layers was washed with brine, dried over Na₂SO₄, filtered and concentrated in vacuo. The residue was purified by silica gel chromatography eluting with a gradient of EtOAc (0 to 90%) in hexanes to provide 4-bromo-2-fluoro-5-((1,1,1-trifluoropropan-2-yl)amino)benzonitrile (550 mg, 76% yield). MS: [M+H]+: 313.0.

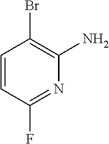

### Intermediate AQ (3-bromo-6-fluoropyridin-2-amine)

A 20 mL MW vessel was charged with 3-bromo-2,6-difluoro-pyridine (1.0 g, 5.2 mmol) and 7 N ammonia solution in MeOH (3.7 mL, 25.9 mmol). The vessel was sealed and heated at 65° C. overnight. The reaction mixture was concentrated and the residue was redissolved in EtOAc and saturated aqueous NaHCO₃ solution. The aqueous layer was cut and the organic layer was washed with brine, dried over MgSO₄, filtered and concentrated. The residue was purified by silica gel chromatography eluting with a gradient of EtOAc (0 to 30%) in heptane to provide 3-bromo-6-fluoro-pyridin-2-amine (554 mg, 56% yield). MS: [M+H]+: 191.0/193.0; ¹H NMR (Chloroform-d) δ: 7.67 (ddd, J=8.0, 7.2, 0.7 Hz, 1H), 6.17 (ddd, J=8.3, 2.8, 0.7 Hz, 1H), 4.96 (br s, 2H).

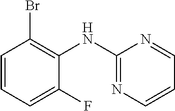 [SMILES: Fc1cccc(Br)c1Nc1ncccn1]

### Intermediate AR (N-(2-bromo-6-fluorophenyl)pyrimidin-2-amine)

To a mixture of 2-bromo-6-fluoro-aniline (507 mg, 2.67 mmol) and 2-chloropyrimidine (611 mg, 5.34 mmol) in trifluoroethanol (5 mL) was added TFA (617 μL, 8.01 mmol). The vial was capped and transferred to a preheated (100° C.) heat block and stirred for 8 h. The cooled reaction mixture was diluted with aqueous saturated NaHCO₃ solution and extracted with DCM (3×). The combined extracts was dried over Na₂SO₄, filtered and concentrated. The residue was purified by silica gel chromatography eluting with a gradient of EtOAc (0 to 60%) in heptane to provide N-(2-bromo-6-fluoro-phenyl)pyrimidin-2-amine (137 mg, 19% yield). MS: [M+H]+: 268.0.

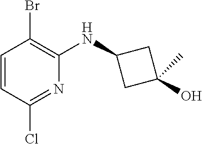

### Intermediate AS ((1s,3s)-3-((3-bromo-6-chloropyridin-2-yl)amino)-1-methylcyclobutan-1-ol)

To a solution of 3-bromo-6-chloro-2-fluoro-pyridine (530 mg, 2.52 mmol) and K₂CO₃ (870 mg, 6.29 mmol) in DMSO (2 mL) was added (1s,3s)-3-amino-1-methylcyclobutan-1-ol hydrochloride (350 mg, 2.54 mmoll) at rt. The mixture was stirred at rt 2 h then poured into water and extracted with ether. The organic layer was washed with water and brine, dried over MgSO₄ and concentrated. The residue was purified by silica gel chromatography eluting with a gradient of EtOAc (0 to 65%) in heptane to provide (1s,3s)-3-((3-bromo-6-chloropyridin-2-yl)amino)-1-methylcyclobutan-1-ol (540 mg, 74% yield). MS: [M+H]+: 293.0.

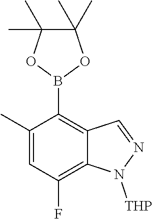

### Intermediate AT (7-fluoro-5-methyl-1-(tetrahydro-2H-pyran-2-yl)-4-(4,4,5,5-tetramethyl-1,3,2-dioxaborolan-2-yl)-1H-indazole)

Step 1. A RBF was charged with 2,2,6,6-tetramethylpiperidine (7.20 mL, 42.7 mmol,) and THF (60 mL). The mixture was cooled to −20° C. and then treated with 2.5 M solution of n-butyllithium in hexanes (17 mL, 42.5 mmol). The mixture was stirred at 0° C. for 30 min then cooled to −78° C. and treated with 1-bromo-4,5-difluoro-2-methylbenzene (8.03 g, 38.8 mmol) dissolved in THF (35 mL), keeping the internal temperature below −60° C. The mixture was stirred for 1 h at −78° C. then treated with dimethylformamide (3.3 mL, 42.6 mmol) diluted in THF (6 mL). After the addition, the cooling bath was removed and the mixture was allowed to warm-up to −20° C. The reaction mixture was quenched with 70 mL of 10% aqueous solution of HCl and then diluted with 50 mL of EtOAc. The aqueous layer was cut and extracted with 50 mL of EtOAc. The combined organic layer was washed with brine, dried over Na₂SO₄, filtered and concentrated to give 2-bromo-5,6-difluoro-3-methyl-benzaldehyde (9.18 g, 99% yield). ¹H NMR (Chloroform-d) δ: 10.35 (dd, J=1.8, 1.0 Hz, 1H), 7.31 (dd, J=10.4, 7.9 Hz, 1H), 2.43 (d, J=1.1 Hz, 3H).

Step 2. A RBF was charged with 2-bromo-5,6-difluoro-3-methyl-benzaldehyde (10.4 g, 44.3 mmol) and DMSO (100 mL). The solution was then treated with hydrazine hydrate (26 mL, 535 mmol). The mixture was stirred at 120° C. for 8 h. The cooled reaction mixture was slowly added to 900 mL of rapidly stirring water. After one hour of stirring, the mixture was acidified with concentrated HCl to pH 5 and the solids were filtered. The filter cake was washed with water and heptane consecutively and dried overnight under high vacuum to provide 4-bromo-7-fluoro-5-methyl-1H-indazole (8.9 g, 87% yield). MS: [M+H]+: 228.9/230.9.

Step 3. 3,4-dihydro-2H-pyran (6 mL, 66.1 mmol) was added to a solution of 4-bromo-7-fluoro-5-methyl-1H-indazole (7.35 g, 32.1 mmol) and p-toluenesulfonic acid (553 mg, 3.2 mmol) in EtOAc (120 mL) and the mixture was stirred at reflux for 3 h. The mixture was filtered and the volatiles were removed under vacuum. The residue residue was purified by silica gel chromatography eluting with a gradient of EtOAc (0 to 10%) in heptane to provide 4-bromo-7-fluoro-5-methyl-1-tetrahydropyran-2-yl-indazole (6.0 g, 60% yield). MS: [M−THP+H]+: 228.5/230.5. ¹H NMR (Chloroform-d) δ: 7.99 (d, J=1.9 Hz, 1H), 6.96 (d, J=12.1 Hz, 1H), 5.80 (dd, J=10.0, 2.6 Hz, 1H), 4.02 (ddt, J=11.8, 4.1, 2.1 Hz, 1H), 3.72 (td, J=11.3, 2.7 Hz, 1H), 2.64-2.48 (m, 1H), 2.44 (s, 3H), 2.17-2.10 (m, 1H), 2.07 (dt, J=12.9, 3.0 Hz, 1H), 1.80-1.65 (m, 2H), 1.65-1.54 (m, 1H)

Step 4. A pressure vessel was charged with a solution of 4-bromo-7-fluoro-5-methyl-1-tetrahydropyran-2-yl-indazole (7.83 g, 25.0 mmol) in dioxane (50 mL). Bis(pinacolato)diboron (7.62 g, 30.0 mmol) was added followed by KOAc (7.36 g, 75.0 mmol) and Pd(dppf)Cl₂·DCM (1.02 g, 1.3 mmol). The vessel was flushed with N₂, sealed and stirred at 100° C. for 4 h. The cooled reaction mixture was filtered through Celite™ (rinsed with EtOAc) and concentrated. The residue was purified by silica gel chromatography eluting with a gradient of EtOAc (0 to 20%) in heptane to provide 7-fluoro-5-methyl-1-tetrahydropyran-2-yl-4-(4,4,5,5-tetramethyl-1,3,2-dioxaborolan-2-yl)indazole (8.12 g, 90% yield). MS: [M+H]+: 361.0.

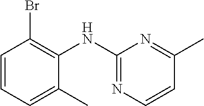 [SMILES: Cc1ccnc(Nc2c(C)cccc2Br)n1]

### Intermediate AU (N-(2-bromo-6-methylphenyl)-4-methylpyrimidin-2-amine)

To a MW vial charged with 2-bromo-6-methyl-aniline (515 mg, 2.77 mmol) and 2-chloro-4-methyl-pyrimidine (702 mg, 5.46 mmol) was added trifluoroethanol (5 mL) and TFA (640 μL, 8.31 mmol). The vial was capped and submitted to microwave irradiation at 100° C. for 2 h. The reaction mixture was concentrated then diluted with DCM and aqueous saturated NaHCO₃ solution. The layers were separated and the aqueous layer was extracted with DCM (3×). The combined organic layers was dried over Na₂SO₄, filtered and concentrated. The residue residue was purified by silica gel chromatography eluting with a gradient of EtOAc (0 to 60%) in heptane to provide N-(2-bromo-6-methyl-phenyl)-4-methyl-pyrimidin-2-amine (175 mg, 23% yield). MS: [M+H]+: 280.0.

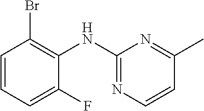 [SMILES: Cc1ccnc(Nc2c(F)cccc2Br)n1]

### Intermediate AV (N-(2-bromo-6-fluorophenyl)-4-methylpyrimidin-2-amine)

To a MW vial charged with 2-bromo-6-fluoro-aniline (513 mg, 2.70 mmol) and 2-chloro-4-methyl-pyrimidine (708 mg, 5.51 mmol) was added trifluoroethanol (5 mL) and TFA (625 μL, 8.11 mmol). The vial was capped and submitted to microwave irradiation at 100° C. for 2 h. The reaction mixture was concentrated then diluted with DCM and aqueous saturated NaHCO₃ solution. The layers were separated and the aqueous layer was extracted with DCM (3×). The combined organic layers was dried over Na₂SO₄, filtered and concentrated. The residue was purified by silica gel chromatography eluting with a gradient of EtOAc (0 to 60%) in heptane to provide N-(2-bromo-6-fluoro-phenyl)-4-methyl-pyrimidin-2-amine (152 mg, 20% yield). MS: [M+H]+: 284.0.

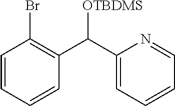 [SMILES: CC(C)(C)[Si](C)(C)OC(c1ccccn1)c1ccccc1Br]

### Intermediate AW (2-((2-bromophenyl)((tert-butyldimethylsilyl)oxy)methyl)pyridine)

To a solution of (2-bromophenyl)-(2-pyridyl)methanol (1.5 g, 5.7 mmol) and imidazole (775 mg, 11.4 mmol) in DMF (20 mL) was added tert-butyldimethylsilyl chloride (1.25 g, 8.3 mmol). After stirring for 18 h, water was added and the mixture was extracted with ether, washed with brine, dried over Na₂SO₄, filtered and concentrated. The residue residue was purified by silica gel chromatography eluting with a gradient of EtOAc (0 to 50%) in heptane to provide 2-((2-bromophenyl)((tert-butyldimethylsilyl)oxy)methyl)pyridine (1.65 g, 77% yield). MS: [M+H]+: 378.2.

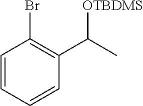 [SMILES: CC(O[Si](C)(C)C(C)(C)C)c1ccccc1Br]

### Intermediate AX ((1-(2-bromophenyl)ethoxy)(tert-butyl)dimethylsilane)

To a solution of 1-(2-bromophenyl)ethanol (2.5 g, 12.4 mmol) and imidazole (1.35 g, 19.8 mmol) in DMF (30 mL) was added tert-butyldimethylsilyl chloride (1.5 g, 10.0 mmol). After stirring for 18 h, water was added and the mixture was extracted with ether, washed with brine and dried over Na₂SO₄, filtered and concentrated. The residue was purified by silica gel chromatography eluting with a gradient of EtOAc (0 to 15%) in heptane to provide 1-(2-bromophenyl)ethoxy-tert-butyl-dimethyl-silane (2.2 g, 88% yield). 1H NMR (400 MHz, Chloroform-d) δ 7.60 (dd, J=7.8, 1.8 Hz, 1H), 7.45 (dd, J=8.0, 1.3 Hz, 1H), 7.30 (td, J=7.5, 1.3 Hz, 1H), 7.06 (ddd, J=7.9, 7.3, 1.8 Hz, 1H), 5.15 (q, J=6.2 Hz, 1H), 1.37 (d, J=6.2 Hz, 3H), 0.89 (s, 9H), 0.05 (s, 3H), −0.05 (s, 3H).

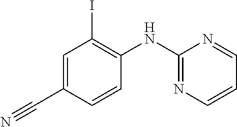 [SMILES: N#Cc1ccc(Nc2ncccn2)c(I)c1]

### Intermediate AY (3-iodo-4-(pyrimidin-2-ylamino)benzonitrile)

To a MW vial charged with 4-amino-3-iodo-benzonitrile (996 mg, 4.1 mmol), 2-fluoropyrimidine (710 mg, 7.2 mmol) and K₂CO₃ (1.73 g, 12.5 mmol) was added DMF (10 mL). The vial was capped and transferred to a preheated (110° C.) heat block and stirred overnight. The reaction mixture was cooled to rt, diluted with water and stirred. The solids were collected by filtration, washed with water and dried affording 3-iodo-4-(pyrimidin-2-ylamino)benzonitrile (1.09 g, 83% yield). MS: [M+H]+: 323.1.

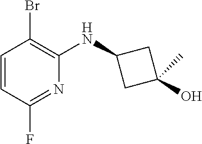

### Intermediate AZ ((1s,3s)-3-((3-bromo-6-fluoropyridin-2-yl)amino)-1-methylcyclobutan-1-ol)

To a solution of 3-bromo-2,6-difluoro-pyridine (200 mg, 1.0 mmol) and K₂CO₃ (360 mg, 2.6 mmol) in DMSO (1 mL) was added (1s,3s)-3-amino-1-methylcyclobutan-1-ol hydrochloride (142 mg, 1.03 mmol) at rt. The mixture was stirred at rt 2 h then poured into water and extracted with ether. The organic layer was washed with water and brine, dried over MgSO₄ and concentrated. The residue was purified by silica gel chromatography eluting with a gradient of EtOAc (0 to 65%) in heptane to provide (1s,3s)-3-((3-bromo-6-fluoropyridin-2-yl)amino)-1-methylcyclobutan-1-ol (197 mg, 69% yield).

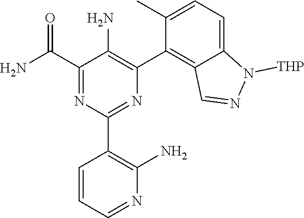

### Intermediate BA (5-amino-2-(2-aminopyridin-3-yl)-6-(5-methyl-1-(tetrahydro-2H-pyran-2-yl)-1H-indazol-4-yl)pyrimidine-4-carboxamide)

Step 1. A MW vial was loaded with Intermediate AB (250 mg, 601.2 μmol), 3-(4,4,5,5-tetramethyl-1,3,2-dioxaborolan-2-yl)pyridin-2-amine (170 mg, 772.5 μmol) and Pd(dppf)Cl₂·DCM (20 mg, 30.7 μmol). Dioxane (3 mL) and 2 M aqueous solution of K₂CO₃ (750 μL, 1.5 mmol) were added. The vial was flushed with N₂, sealed and stirred at 110° C. for 90 min. The reaction mixture was diluted with EtOAc and water and filtered through a pad of Celite™. The aqueous layer was cut and the organic layer was washed with brine, dried over MgSO₄, filtered and concentrated. The residue was purified by silica gel chromatography eluting with a gradient of EtOAc (50 to 100%) in heptane to provide ethyl 5-amino-2-(2-amino-3-pyridyl)-6-(5-methyl-1-tetrahydropyran-2-yl-indazol-4-yl)pyrimidine-4-carboxylate (217 mg, 76% yield). MS: [M+H]+: 474.2.

Step 2. To a solution of ethyl 5-amino-2-(2-amino-3-pyridyl)-6-(5-methyl-1-tetrahydropyran-2-yl-indazol-4-yl)pyrimidine-4-carboxylate (120 mg, 253.4 μmol) in MeOH (1 mL) were added 7 N ammonia solution in MeOH (1.5 mL, 10.5 mmol). The mixture was stirred at 80° C. for 2 h in a sealed vial. The volatiles were removed in vacuo to provide 5-amino-2-(2-amino-3-pyridyl)-6-(5-methyl-1-tetrahydropyran-2-yl-indazol-4-yl)pyrimidine-4-carboxamide (110 mg, 247.48 μmol, 98% yield). MS: [M+H]+: 464.3.

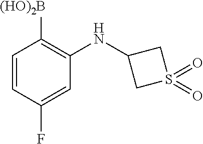 [SMILES: O=S1(=O)CC(Nc2cc(F)ccc2B(O)O)C1]

### Intermediate BB ((2-((1,1-dioxidothietan-3-yl)amino)-4-fluorophenyl)boronic acid)

Step 1. To a solution of 2-bromo-5-fluoroaniline (2.00 g, 10.3 mmol) in DCM (20 mL) at rt was added thietan-3-one (1.86 g, 20.6 mmol), sodium triacetoxyborohydride (4.37 g, 20.6 mmol) and acetic acid (1.77 mL, 30.9 mmol). The reaction mixture was stirred at rt for 3 days. The reaction mixture was slowly poured into aqueous saturated NaHCO₃. The layers were partitioned and the aqueous layer was extracted with DCM. The combined organic layers was washed with brine, dried over MgSO₄, filtered and concentrated. The residue was purified by silica gel chromatography eluting with a gradient of 15% EtOAc/hexanes (0 to 100%) in hexanes to provide N-(2-bromo-5-fluorophenyl)thietan-3-amine (1.27 g, 47% yield). MS: [M+H]+: 261.9/263.9.

Step 2. To a solution of N-(2-bromo-5-fluorophenyl)thietan-3-amine (1.33 g, 5.07 mmol) in EtOAc (13.0 mL) were added sodium tungstate dihydrate (83.7 mg, 254 μmol) and tetrabutylammonium hydrogensulfate (138 mg, 406 μmol). The reaction mixture was cooled to 0° C. and hydrogen peroxide (3.89 mL, 38.1 mmol) was added dropwise. The reaction mixture was stirred at rt for 3 h. Additional hydrogen peroxide (1.5 mL) was added and the mixture was stirred for additional 1 h. The reaction mixture was diluted with EtOAc and 10% aqueous NaHSO₃. The layers were partitioned and the aqueous layer was extracted with EtOAc. The combined organic layers was washed with brine, dried over MgSO₄, filtered and concentrated. The residue was purified by silica gel chromatography eluting with a gradient of EtOAc (50 to 60%) in hexanes to provide 3-((2-bromo-5-fluorophenyl)amino)thietane 1,1-dioxide (1.1 g, 74% yield). MS: [M+H]+: 294.0/296.0. ¹H-NMR (400 MHz, CDCl₃): δ 7.43 (dd; J=8.74; 5.87 Hz; 1H); 6.47 (td; J=8.36; 2.76 Hz; 1H); 6.18 (dd; J=10.44; 2.76 Hz; 1H); 4.82-4.85 (m; 1H); 4.57-4.63 (m; 2H); 4.23-4.29 (m; 1H); 4.00-4.05 (m; 2H).

Step 3. 3-((2-bromo-5-fluorophenyl)amino)thietane 1,1-dioxide (125 mg, 425 μmol), Xphos Pd G3 (18.0 mg, 21.2 μmol), potassium acetate (125 mg, 1.27 mmol) and tetrahydroxydiboron (114 mg, 1.27 mmol) were loaded into a microwave reaction vial and capped. The mixture was degassed (3 cycles of vacuum/argon atmosphere). MeOH (1.50 mL) and ethylene glycol (500 μL) were added and the reaction mixture was heated to 60° C. for 18 h. The mixture was filtered through Celite™ and washed with EtOAc. The volatiles were removed in vacuo to afford crude (2-((1,1-dioxidothietan-3-yl)amino)-4-fluorophenyl)boronic acid which was used as such in the subsequent step without further purification.

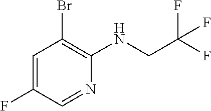 [SMILES: Fc1cnc(NCC(F)(F)F)c(Br)c1]

### Intermediate BC (3-bromo-5-fluoro-N-(2,2,2-trifluoroethyl)pyridin-2-amine)

Step 1. Aflame dried microwave tube was loaded with THF (3.0 mL), DMF (500 μL) and 2-amino-3-bromo-5-fluoropyridine (200 mg, 1.05 mmol). The reaction mixture was heated at 80° C., phenylsilane (323 μL, 2.62 mmol) was added followed by TFA (96 μL, 1.26 mmol). The reaction was kept at 80° C. for 18 h. Upon cooling to rt, the volatiles were removed in vacuo. The residue was dissolved in EtOAc and washed with saturated aqueous sodium bicarbonate. The organic layer was dried over MgSO₄, filtered and concentrated. The residue was purified by silica gel chromatography eluting with a gradient of EtOAc (0 to 90%) in hexanes to provide N-(3-bromo-5-fluoropyridin-2-yl)-2,2,2-trifluoroacetamide (250 mg, 83% yield). MS: [M+H]+: 286.6.

Step 2. To a solution of N-(3-bromo-5-fluoropyridin-2-yl)-2,2,2-trifluoroacetamide (500 mg, 1.74 mmol) in THF (10 mL) was added 2 M solution of BH3·DMS in THF (1.74 mL, 3.48 mmol). The reaction mixture was heated to 60° C. for 24 h. The cooled reaction mixture was diluted with water, extracted with EtOAc (2×). The combined organic extracts was dried over MgSO₄, filtered and concentrated to dryness. The residue was purified by silica gel chromatography eluting with a gradient of EtOAc (0 to 90%) in hexanes to provide 3-bromo-5-fluoro-N-(2,2,2-trifluoroethyl) yridine-2-amine (400 mg, 84% yield). MS: [M+H]+: 272.6/274.4.

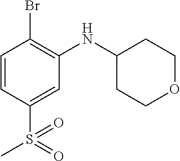 [SMILES: CS(=O)(=O)c1ccc(Br)c(NC2CCOCC2)c1]

### Intermediate BD (N-(2-bromo-5-(methylsulfonyl)phenyl)tetrahydro-2H-pyran-4-amine)

A RBF was charged with tetrahydropyran-4-one (700 mg, 7.00 mmol) and THF (10 mL). Concentrated sulfuric acid (2.25 mL) was added followed by 2-bromo-5-methylsulfonyl-aniline (250 mg, 1.00 mmol). The mixture was stirred at rt for 1 h then cooled to 0° C. and treated with sodium borohydride (265 mg, 7.00 mmol). The mixture was stirred overnight at rt. The reaction mixture was diluted with EtOAc and basicified with 1 N aqueous solution of KOH. The aqueous layer was cut and extracted (2×) with EtOAc. The combined organic layer was washed with brine, dried over Na₂SO₄, filtered and concentrated. The residue was purified by silica gel chromatography eluting with a gradient of EtOAc (30 to 100%) in heptane to provide N-(2-bromo-5-methylsulfonyl-phenyl)tetrahydropyran-4-amine (89 mg, 27% yield). MS: [M+H]+: 334.1/336.1.

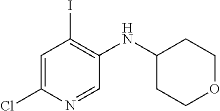 [SMILES: Clc1cc(I)c(NC2CCOCC2)cn1]

### Intermediate BE (6-chloro-4-iodo-N-(tetrahydro-2H-pyran-4-yl)pyridin-3-amine)

A 4 mL vial was charged with 6-chloro-4-iodo-pyridin-3-amine (100 mg, 393.0 μmol), EtOAc (1.2 mL), tetrahydropyran-4-one (47 μL, 507.1 μmol) and TFA (62.00 μL, 804.8 μmol). Sodium triacetoxyborohydride (106 mg, 500.1 μmol) was added and the mixture was stirred at rt for 1 h. The reaction mixture was diluted with EtOAc and water and the mixture was neutralized with saturated aqueous NaHCO₃ solution. The aqueous layer was cut and the organic layer was washed with brine, dried over MgSO₄, filtered and concentrated. The residue was purified by silica gel chromatography eluting with a gradient of EtOAc (0 to 50%) in heptane to provide 6-chloro-4-iodo-N-tetrahydropyran-4-yl-pyridin-3-amine (86 mg, 65% yield). MS: [M+H]+: 339.1/341.0.

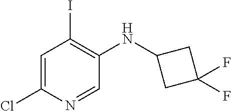 [SMILES: FC1(F)CC(Nc2cnc(Cl)cc2I)C1]

### Intermediate BF (6-chloro-N-(3,3-difluorocyclobutyl)-4-iodopyridin-3-amine)

A 4 mL vial was charged with 6-chloro-4-iodo-pyridin-3-amine (100 mg, 393.0 μmol), EtOAc (1.2 mL) 3,3-difluorocyclobutanone (54.19 mg, 510.89 μmol) and TFA (62.00 μL, 804.8 μmol). Sodium triacetoxyborohydride (106 mg, 500.1 μmol) was added and the mixture was stirred at rt for 1 h. The reaction mixture was diluted with EtOAc and water and the mixture was neutralized with saturated aqueous NaHCO₃ solution. The aqueous layer was cut and the organic layer was washed with brine, dried over MgSO₄, filtered and concentrated. The residue was purified by silica gel chromatography eluting with a gradient of EtOAc (0 to 50%) in heptane to provide 6-chloro-N-(3,3-difluorocyclobutyl)-4-iodopyridin-3-amine (53 mg, 39% yield). MS: [M+H]+: 345.0/347.0.

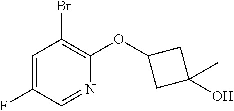

### Intermediate BG (3-((3-bromo-5-fluoropyridin-2-yl)oxy)-1-methylcyclobutan-1-ol)

Step 1. A RBF was charged with 3-(benzyloxy)cyclobutanone (500 mg, 2.75 mmol) and dry toluene (13.8 mL). Methylmagnesium bromide (3 M in Et₂O, 1.10 mL, 3.30 mmol) was slowly added to the reaction mixture at −78° C. under a nitrogen atmosphere. After 2 h, MeOH was added and the slurry was filtered on Celite™ and washed with EtOAc. The mixture was concentrated and the residue was purified by silica gel chromatography eluting with a gradient of EtOAc (0 to 40%) in hexanes to provide 3-(benzyloxy)-1-methylcyclobutan-1-ol (270 mg, 51% yield). MS: [M-OH]+: 175.2.

Step 2. A mixture of 3-(benzyloxy)-1-methylcyclobutan-1-ol (120 mg, 624 μmol) and palladium on carbon 10 wt. % (60 mg, 567 μmol) in MeOH (5.7 mL) was stirred under an atmosphere of hydrogen for 16 h. The reaction mixture was filtered on Celite™ and concentrated. The residue was purified by silica gel chromatography eluting with a gradient of EtOAc (0 to 40%) in hexanes to provide 1-methylcyclobutane-1,3-diol (30 mg, 47% yield).

Step 3. To 3-Bromo-2,5-difluoropyridine (43 μL, 401 μmol) and 1-methylcyclobutane-1,3-diol (45.0 mg, 441 μmol) in dry THF (1 mL) was added sodium hydride (60%, 19 mg, 481 μmol). The mixture was heated to 50° C. for 2 h. The cooled reaction mixture was quenched with aqueous saturated NaHCO₃ solution and the aqueous layer was extracted with DCM (2×). Organic layers were combined, washed with brine, dried over Na₂SO₄, filtered and concentrated. The residue was purified by silica gel chromatography eluting with a gradient of EtOAc (0 to 100%) in hexanes to provide 3-((3-bromo-5-fluoropyridin-2-yl)oxy)-1-methylcyclobutan-1-ol (50 mg, 45% yield). MS: [M+H]+: 276.1.

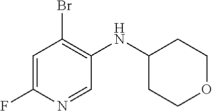 [SMILES: Fc1cc(Br)c(NC2CCOCC2)cn1]

### Intermediate BH (4-bromo-6-fluoro-N-(tetrahydro-2H-pyran-4-yl)pyridin-3-amine)

A 4 mL vial was charged with 4-bromo-6-fluoro-pyridin-3-amine (150 mg, 785.3 μmol), EtOAc (1.2 mL), tetrahydropyran-4-one (94 μL, 1.01 mmol,) and TFA (124 μL, 1.61 mmol). Sodium triacetoxyborohydride (212 mg, 1.00 mmol) was added and the mixture was stirred at rt for 1 h. The reaction mixture was diluted with EtOAc and water and the mixture was neutralized with saturated aqueous NaHCO₃ solution. The aqueous layer was cut and the organic layer was washed with brine, dried over MgSO₄, filtered and concentrated. The residue was purified by silica gel chromatography eluting with a gradient of EtOAc (0 to 30%) in heptane to provide 4-bromo-6-fluoro-N-tetrahydropyran-4-yl-pyridin-3-amine (198 mg, 92% yield). MS: [M+H]+: 275.1/277.1.

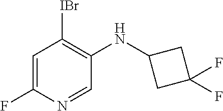

### Intermediate BI (4-bromo-N-(3,3-difluorocyclobutyl)-6-fluoropyridin-3-amine)

A 4 mL vial was charged with 4-bromo-6-fluoro-pyridin-3-amine (150 mg, 785.3 μmol), EtOAc (1.2 mL), 3,3-difluorocyclobutanone (108 mg, 1.01 mmol) and TFA (124 μL, 1.61 mmol). Sodium triacetoxyborohydride (212 mg, 1.00 mmol) was added and the mixture was stirred at rt for 1 h. The reaction mixture was diluted with EtOAc and water and the mixture was neutralized with saturated aqueous NaHCO₃ solution. The aqueous layer was cut and the organic layer was washed with brine, dried over MgSO₄, filtered and concentrated. The residue was purified by silica gel chromatography eluting with a gradient of EtOAc (0 to 30%) in heptane to provide 4-bromo-N-(3,3-difluorocyclobutyl)-6-fluoropyridin-3-amine (146 mg, 66% yield). MS: [M+H]+: 281.0/283.0.

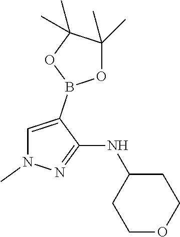 [SMILES: Cn1cc(B2OC(C)(C)C(C)(C)O2)c(NC2CCOCC2)n1]

### Intermediate BJ (1-methyl-N-(tetrahydro-2H-pyran-4-yl)-4-(4,4,5,5-tetramethyl-1,3,2-dioxaborolan-2-yl)-1H-pyrazol-3-amine)

Step 1. A RBF was charged with 4-bromo-1-methyl-pyrazol-3-amine (500 mg, 2.84 mmol), IPAc (10 mL), tetrahydropyran-4-one (340 μL, 3.67 mmol) and TFA (445 μL, 5.78 mmol). The mixture treated with sodium triacetoxyborohydride (789 mg, 3.72 mmol) and stirred at rt for 90 min. The reaction mixture was diluted with EtOAc and water and neutralized with saturated aqueous NaHCO₃ solution. The aqueous layer was cut and the organic layer was washed with brine, dried over MgSO₄, filtered and concentrated. The residue was purified by silica gel chromatography eluting with a gradient of EtOAc (50 to 100%) in heptane to provide 4-bromo-1-methyl-N-tetrahydropyran-4-yl-pyrazol-3-amine (622 mg, 84% yield). MS: [M+H]+: 259.9/261.9.

Step 2. A RBF was charged with 4-bromo-1-methyl-N-tetrahydropyran-4-yl-pyrazol-3-amine (622 mg, 2.39 mmol), potassium 2-ethylhexanoate (959 mg, 5.26 mmol), bis(pinacolato)diboron (729 mg, 2.87 mmol), dicyclohexyl-[2-(2,4,6-triisopropylphenyl)phenyl]phosphane (57 mg, 119.6 μmol) and IPAc (12 mL). The mixture was stirred at 40° C. for 10 min under N₂ then XPhos Pd(allyl)Cl (79 mg, 119.6 μmol) was added under N₂. The mixture was stirred at 40° C. for 24 h. The reaction mixture was diluted with EtOAc and water. The aqueous layer was cut and the organic layer was washed with brine, dried over MgSO₄, filtered and concentrated. The residue was purified by silica gel chromatography eluting with a gradient of EtOAc (50 to 100%) in heptane to provide 1-methyl-N-tetrahydropyran-4-yl-4-(4,4,5,5-tetramethyl-1,3,2-dioxaborolan-2-yl)pyrazol-3-amine (785 mg, 50% yield).

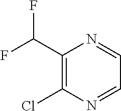

### Intermediate BK (2-chloro-3-(difluoromethyl)pyrazine)

To a solution of triethylamine trihydrofluoride (2.1 mL, 12.88 mmol) in DCM (10 mL) were added XtalFluor-E (2.18 g, 9.52 mmol), followed by 3-chloropyrazine-2-carbaldehyde (890 mg, 6.33 mmol). The mixture was stirred at rt for 2 h then quenched by addition of aqueous saturated NaHCO₃ solution, diluted with water and extracted with DCM (3×20 mL). The combined organic extracts were washed with bine, dried over sodium sulfate, filtered and concentrated. The residue was purified by silica gel chromatography eluting with a gradient of EtOAc (0 to 30%) in heptane to provide 2-chloro-3-(difluoromethyl)pyrazine (700 mg, 67% yield). ¹H NMR (400 MHz, Chloroform-d) δ 8.59 (d, J=2.4 Hz, 1H), 8.52 (dt, J=2.2, 1.0 Hz, 1H), 6.89 (m, 1H).

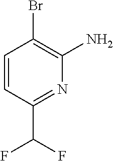 [SMILES: Nc1nc(C(F)F)ccc1Br]

### Intermediate BL (3-bromo-6-(difluoromethyl)pyridin-2-amine)

Step 1. To a solution of methyl 6-amino-5-bromo-pyridine-2-carboxylate (2.5 g, 10.8 mmol) in THF (25 mL) was added LiBH₄ (250 mg, 11.5 mmol). The mixture was stirred at rt for 18 h. The reaction was quenched by addition of water and the mixture was diluted with water, extracted with CHCl₃/IPA (4:1) (4×30 mL). The combined organic extracts were washed with brine, dried over sodium sulfate and concentrated to dryness to provide (6-amino-5-bromo-2-pyridyl)methanol (2.0 g, 91% yield) as a white solid that was used without purification.

Step 2. To a solution of (6-amino-5-bromo-2-pyridyl)methanol (1.94 g, 9.55 mmol) in DCM (40 mL) was added Manganese(IV) oxide, activated (10 g, 115.03 mmol) in one batch. The mixture was stirred at rt for 3 h then filtered. The filtrate was concentrated to dryness to provide 6-amino-5-bromo-pyridine-2-carbaldehyde (1.5 g, 78% yield).

Step 3. To a solution of triethylamine trihydrofluoride (7.22 mL, 44.32 mmol) in DCM (120 mL) were added XtalFluor-E (9.89 g, 43.19 mmol), followed by 6-amino-5-bromo-pyridine-2-carbaldehyde (4.3 g, 21.39 mmol). The mixture was stirred at rt for 2 h then quenched by addition of aqueous saturated NaHCO₃ solution, diluted with water and extracted with DCM (3×100 mL). The organic extracts were washed with bine, dried over sodium sulfate, filtered and concentrated in vacuo. The residue was purified by silica gel chromatography eluting with a gradient of EtOAc (0 to 30%) in heptane to provide 3-bromo-6-(difluoromethyl)pyridin-2-amine (2.1 g, 44% yield). 1H NMR (400 MHz, Chloroform-d) δ 7.74 (d, J=7.8 Hz, 1H), 6.82 (d, J=7.9 Hz, 1H), 6.38 (t, J=55.6 Hz, 1H), 5.08 (s, 2H).

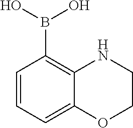 [SMILES: OB(O)c1cccc2c1NCCO2]

### Intermediate BM ((3,4-dihydro-2H-benzo[b][1,4]oxazin-5-yl)boronic acid)

5-Bromo-3,4-dihydro-2H-benzo[b][1,4]oxazine (150 mg, 0.666 mmol), bis(neopentyl glycolato)diboron (317 mg, 1.33 mmol), potassium acetate (198 mg, 2.0 mmol) and PdCl₂(dppf) (48.7 mg, 0.067 mmol) and dioxane (5 mL) were added to a vial fitted with a stir bar. The mixture was degassed with nitrogen 5 min, the vial sealed and heated to 80° C. for 18 h with stirring. The reaction was cooled to ambient temperature and the solvent removed in vacuo. The residue was taken up in DMSO and purified by reverse phase flash chromatography (C18 silica with Water/ACN: 10%-100%) to provide (3,4-dihydro-2H-benzo[b][1,4]oxazin-5-yl)boronic acid. (100 mg, 84% yield). MS: [M+H]+: 180.0.

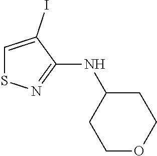 [SMILES: Ic1csnc1NC1CCOCC1]

### Intermediate BN (4-iodo-N-(tetrahydro-2H-pyran-4-yl)isothiazol-3-amine)

Step 1. Isothiazol-5-amine hydrochloride (100 mg, 0.695 mmol), tetrahydro-4H-pyran-4-one (57 μL, 0.835 mmol), sodium triacetoxyborohydride (220 mg, 1.04 mmol), TFA (107 μL, 1.39 mmol) and isopropyl acetate (3.3 mL) were added to a vial fitted with a stir bar. The mixture was sonicated for 2 min and stirred at ambient temperature 16 h. The reaction was diluted with EtOAc and triethylamine (500 μL, 3.55 mmol) was added. The mixture was adsorbed directly onto silica gel. The residue was purified by silica gel chromatography eluting with a gradient of i-PrOH (1 to 30%) in DCM to provide N-(tetrahydro-2H-pyran-4-yl)isothiazol-5-amine which was taken up in DMF (3 mL) and N-iodosuccinimide (192 mg, 835 μmol) was added. The reaction was stirred at room temp 30 min, diluted with EtOAc and adsorbed onto silica gel. The residue was purified by silica gel chromatography eluting with a gradient of EtOAc (2 to 80%) in DCM to provide 4-iodo-N-(tetrahydro-2H-pyran-4-yl)isothiazol-5-amine (55.0 mg, 25% yield). MS: [M+H]+: 310.9.

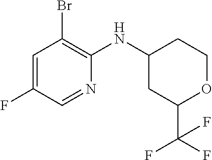 [SMILES: Fc1cnc(NC2CCOC(C(F)(F)F)C2)c(Br)c1]

### Intermediate BO (3-bromo-5-fluoro-N-(2-(trifluoromethyl)tetrahydro-2H-pyran-4-yl)pyridin-2-amine)

To a solution of 3-bromo-5-fluoro-pyridin-2-amine (231 mg, 1.21 mmol) in EtOAc (5 mL) was added 2-(trifluoromethyl)tetrahydropyran-4-one (256 mg, 1.52 mmol) and TFA (186 μL, 2.41 mmol). Sodium triacetoxyborohydride (384 mg, 1.81 mmol) was added and the mixture was stirred at rt for 4 days. Saturated aqueous solution of NaHCO₃ was added slowly and the mixture was stirred vigorously. The layers were separated and the aqueous layer was extracted with EtOAc (2×). The combined organic layers was washed with brine, dried over Na₂SO₄, filtered and concentrated. The residue was purified by silica gel chromatography eluting with a gradient of EtOAc (0 to 70%) in heptane to provide 3-bromo-5-fluoro-N-[2-(trifluoromethyl)tetrahydropyran-4-yl]pyridin-2-amine (179 mg, 43% yield). MS: [M+H]+: 344.9.

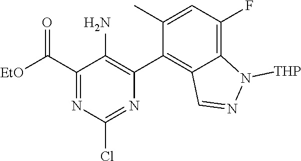

### Intermediate BP (ethyl 5-amino-2-chloro-6-(7-fluoro-5-methyl-1-(tetrahydro-2H-pyran-2-yl)-1H-indazol-4-yl)pyrimidine-4-carboxylate)

A pressure vessel was charged with ethyl 5-amino-2,6-dichloro-pyrimidine-4-carboxylate (4.57 g, 19.4 mmol) and a 0.225 M dioxane solution of Intermediate AT (80 mL, 18 mmol). 2 M Aqueous solution of K₃PO₄ (20 mL, 40 mmol) was added followed by Pd(dtbpf)Cl₂ (631 mg, 968 μmol). The vessel was flushed with N₂, sealed and stirred at 85° C. for 1 h. The cooled reaction mixture was diluted with water and EtOAc and filtered through Celite™. The filtrate was further diluted with EtOAc and water then saturated with NaCl. The aqueous layer was cut and the organic layer was washed with brine, dried over MgSO₄, filtered and concentrated. The residue was purified by silica gel chromatography eluting with a gradient of EtOAc (20 to 50%) in heptane to provide ethyl 5-amino-2-chloro-6-(7-fluoro-5-methyl-1-tetrahydropyran-2-yl-indazol-4-yl)pyrimidine-4-carboxylate (5.15 g, 61% yield). MS: [M+H]+: 434.0/436.0.

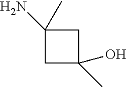

### Intermediate BQ (3-amino-1,3-dimethylcyclobutan-1-ol)

Step 1. A RBF under inert atmosphere, was charged with 1-methyl-3-oxocyclobutanecarboxylic acid (1.0 g, 7.8 mmol) and dry THF (16 mL). The mixture was cooled to 0° C. and methylmagnesium bromide (1.4 M in THF, 12.3 mL, 17.2 mmol) was added dropwise. The reaction mixture was stirred at rt for 2 h. The reaction was acidified to pH 1-2 with 1 N aqueous HCl solution and EtOAc (10 mL) was added. The layers were partitioned and the aqueous phase was extracted 2 times with EtOAc (10 mL). The combined organic layers was dried over Na₂SO₄, filtered and concentrated. The residue was purified by silica gel chromatography eluting with a gradient of MeOH (1 to 8%) in DCM to provide 3-hydroxy-1,3-dimethylcyclobutane-1-carboxylic acid (610 mg, 54% yield). ¹H-NMR (400 MHz, CDCl3): δ 2.66 (dm, J=13.5 Hz, 2H); 2.06 (dm, J=13.5 Hz, 2H); 1.53 (s, 3H); 1.39 (s, 3H).

Step 2. A solution of triethylamine (590 μL, 4.23 mmol), 3-hydroxy-1,3-dimethylcyclobutane-1-carboxylic acid (610 mg, 4.23 mmol) and diphenylphosphoryl azide (950 μL, 4.23 mmol) in tert-BuOH (21 mL) was heated to reflux for 16 h. Upon cooling to rt, the mixture was diluted with water and extracted with EtOAc (3×). Organic layers were combined, washed with brine, dried over Na₂SO₄, filtered and concentrated. The residue was purified by silica gel chromatography eluting with a gradient of MeOH (0 to 10%) in DCM to provide 1,5-dimethyl-2-oxa-4-azabicyclo[3.1.1]heptan-3-one (450 mg, 75% yield). MS: [M+H]+: 142.0.

Step 3. A mixture of 1,5-dimethyl-2-oxa-4-azabicyclo[3.1.1]heptan-3-one (450 mg, 3.19 mmol), 4 M aqueous solution of KOH (4.78 mL, 19.1 mmol) in isopropanol (16 mL) was heated to 100° C. for 16 h. Upon cooling to rt, the reaction mixture as acidified by adding aqueous HCl solution and concentrated to dryness in vacuo. The residue was triturated with EtOAc and the filtrate was concentrated to dryness to provide 3-amino-1,3-dimethylcyclobutan-1-ol (367 mg, 99% yield).

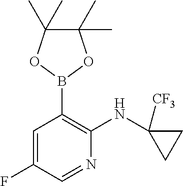 [SMILES: CC1(C)OB(c2cc(F)cnc2NC2(C(F)(F)F)CC2)OC1(C)C]

### Intermediate BR (5-fluoro-3-(4,4,5,5-tetramethyl-1,3,2-dioxaborolan-2-yl)-N-(1-(trifluoromethyl)cyclopropyl)pyridin-2-amine)

Step 1. 2-Bromo-5-fluoropyridine (500 mg, 2.84 mmol), 1-(trifluoromethyl)cyclopropanamine hydrochloride (551 mg, 3.41 mmol), Pd₂(dba)₃ (260 mg, 284 μmol), rac-Binap (354 mg, 568 μmol), sodium tert-butoxide (828 mg, 8.52 mmol) and THF (14 mL) were placed in microwave reaction vial. The reaction mixture was degassed and heated to 50° C. for 16 h. The reaction mixture was diluted with EtOAc and filtered on Celite™ and the filtrate was concentrated. The residue was purified by silica gel chromatography eluting with a gradient of EtOAc (0 to 100%) in hexanes to provide 5-fluoro-N-(1-(trifluoromethyl)cyclopropyl) yridine-2-amine (280 mg, 45% yield). MS: [M+H]+: 221.1.

Step 2. To a solution of 5-fluoro-N-(1-(trifluoromethyl)cyclopropyl)pyridin-2-amine (180 mg, 818 μmol) in DCM (2 mL) and MeOH (2 mL) were added benzyltrimethylammonium tribromide (351 mg, 899 μmol) and calcium carbonate (100 mg, 981 μmol). The reaction stirred at rt for 18 h, filtered on Celite™ and the filter cake was washed with DCM. The filtrate was concentrated and the residue was purified by silica gel chromatography eluting with a gradient of EtOAc (20 to 60%) in hexanes to provide 3-bromo-5-fluoro-N-(1-(trifluoromethyl)cyclopropyl) yridine-2-amine (145 mg, 59% yield). MS: [M+H]+: 299.0.

Step 3. 3-bromo-5-fluoro-N-(1-(trifluoromethyl)cyclopropyl)pyridin-2-amine (13.8 mg, 46.3 μmol), bis(pinacolato)diboron (23.5 mg, 92.5 μmol), PdCl₂(dppf)·DCM (3.8 mg, 4.63 μmol) and potassium acetate (13.8 mg, 139 μmol) in degassed DMF were charged in a microwave vial. The reaction mixture was then heated to 90° C. for 16 h. The reaction mixture was diluted with EtOAc and filtered on Celite™. The filtrate was concentrated to dryness to afford (5-fluoro-2-((1-(trifluoromethyl)cyclopropyl)amino) yridine-3-yl)boronic acid which was used as such in the subsequent step without further purification.

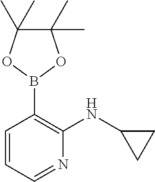 [SMILES: CC1(C)OB(c2cccnc2NC2CC2)OC1(C)C]

### Intermediate BS (N-cyclopropyl-3-(4,4,5,5-tetramethyl-1,3,2-dioxaborolan-2-yl)pyridin-2-amine)

A MW vial was charged with 3-bromo-N-cyclopropyl-pyridin-2-amine (127 mg, 596.0 μmol in dioxane (3 mL). Nitrogen was bubbled through the solution and bis(pinacolato)diboron (194 mg, 764.0 μmol), KOAc (150 mg, 1.53 mmol) and Pd(dppf)Cl₂·DCM (27 mg, 33.1 μmol) were added. The vial was flushed with N₂, sealed and stirred at 110° C. for 1 h to provide N-cyclopropyl-3-(4,4,5,5-tetramethyl-1,3,2-dioxaborolan-2-yl)pyridin-2-amine as a solution in dioxane that would be used as such for a subsequent step.

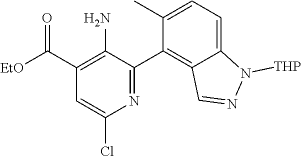

### Intermediate BT (ethyl 3-amino-6-chloro-2-(5-methyl-1-(tetrahydro-2H-pyran-2-yl)-1H-indazol-4-yl)isonicotinate)

Ethyl 3-amino-2-bromo-6-chloroisonicotinate (2.0 g, 7.16 mmol), 5-methyl-1-(tetrahydro-2H-pyran-2-yl)-4-(4,4,5,5-tetramethyl-1,3,2-dioxaborolan-2-yl)-1H-indazole (2.57 g, 7.51 mmol), potassium carbonate (2.47 g, 17.9 mmol), PdCl₂(dppf) (534 mg, 0.716 mmol), dioxane (40 mL) and water (10 mL) were added to a flask fitted with a stir bar. The mixture was degassed with nitrogen 5 min and the flask was fitted with a reflux condenser and heated to 100° C. for 3 h with stirring. The reaction was cooled to rt, filtered through Celite™ with EtOAc as eluent and adsorbed onto silica gel. The residue was purified by silica gel chromatography eluting with a gradient of EtOAc (1 to 40%) in DCM to provide ethyl-3-amino-6-chloro-2-(5-methyl-1-(tetrahydro-2H-pyran-2-yl)-1H-indazol-4-yl) (1.7 g, 57% yield). MS: [M+H]+: 415.3.

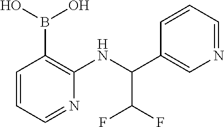 [SMILES: OB(O)c1cccnc1NC(c1cccnc1)C(F)F]

### Intermediate BU ((2-((2,2-difluoro-1-(pyridin-3-yl)ethyl)amino)pyridin-3-yl)boronic acid)

Step 1. To a solution of 4,4,4-trifluoro-1-(pyridin-3-yl)butane-1,3-dione (2.0 g, 8.75 mmol) in acetonitrile (88 mL) was added Selectfluor (7.75 g, 21.9 mmol). The resulting mixture was heated to reflux for 3 h. Then water (18 μL) was added and the reflux was maintained for additional 15 minutes. The reaction mixture was allowed to cool down to rt and triethylamine (6.1 mL) was added. It was stirred at rt for 16 h. The volatiles were removed under vacuum and the residue was purified by silica gel chromatography eluting with a gradient of EtOAc (0 to 100%) in hexanes to provide 2,2-difluoro-1-(yridine-3-yl)ethan-1-one (960 mg, 70% yield).

Step 2. To a solution of 2-amino-3-bromopyridine (308 mg, 1.78 mmol) in DCM (9 mL) was added under argon 2,2-difluoro-1-(pyridin-3-yl)ethan-1-one (420 mg, 2.67 mmol) followed by titanium(IV) chloride (1 M in toluene, 2.14 mL, 2.14 mmol). The resulting mixture was allowed to stir at rt for 6 h. Sodium triacetoxyborohydride (755 mg, 3.56 mmol) was then added and the mixture was stirred at rt for 16 h. The mixture was filtered through Celite™ and washed with EtOAc. The volatiles were removed under vacuum and the residue was purified by silica gel chromatography eluting with a gradient of EtOAc (0 to 100%) in hexanes to provide 3-bromo-N-(2,2-difluoro-1-(pyridine-3-yl)ethyl)pyridine-2-amine (290 mg, 52% yield). MS: [M+H]+: 315.9.

Step 3. 3-Bromo-N-(2,2-difluoro-1-(pyridin-3-yl)ethyl)pyridin-2-amine (50.0 mg, 159 μmol), XPhos Pd G3 (6.74 mg, 7.96 μmol), potassium acetate (46.9 mg, 478 μmol) and tetrahydroxydiboron (42.8 mg, 478 μmol) were loaded into a microwave reaction vial and capped. The mixture was degassed (3 cycles of vacuum/Ar). MeOH (562 μL) and ethylene glycol (187 μL) were added. It was heated to 60° C. for 18 h. The mixture was filtered through Celite™ and washed with MeOH. The volatiles were removed in vacuo and the crude (2-((2,2-difluoro-1-(pyridin-3-yl)ethyl)amino)pyridin-3-yl)boronic acid was used without further purification in the subsequent step.

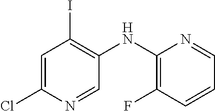 [SMILES: Fc1cccnc1Nc1cnc(Cl)cc1I]

### Intermediate BV (N-(6-chloro-4-iodopyridin-3-yl)-3-fluoropyridin-2-amine)

A RBF was charged with 6-chloro-4-iodo-pyridin-3-amine (100 mg, 393.0 μmol), 2,3-difluoropyridine (45 mg, 393.0 μmol) and DMF (4 mL). Sodium hydride (60% dispersion in mineral oil) (79 mg, 1.96 mmol) was added and the mixture was stirred overnight at rt. The reaction mixture was quenched with aqueous saturated NH₄Cl solution then diluted with EtOAc and water. The aqueous layer was cut and the organic layer was washed with brine, dried over MgSO₄, filtered and concentrated. The residue was purified by silica gel chromatography eluting with a gradient of EtOAc (0 to 30%) in heptane to provide 6-chloro-N-(3-fluoro-2-pyridyl)-4-iodo-pyridin-3-amine (86 mg, 63% yield). MS: [M+H]+: 350.0/351.9.

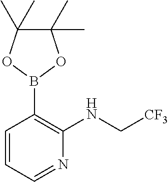 [SMILES: CC1(C)OB(c2cccnc2NCC(F)(F)F)OC1(C)C]

### Intermediate BW (3-(4,4,5,5-tetramethyl-1,3,2-dioxaborolan-2-yl)-N-(2,2,2-trifluoroethyl)pyridin-2-amine)

Step 1. To a MW vial charged with 3-bromo-2-fluoro-pyridine (299 mg, 1.70 mmol) and 2,2,2-trifluoroethanamine (675 μL, 8.52 mmol) in trifluoroethanol (3 mL) was added TFA (392 μL, 5.09 mmol). The vial was capped and stirred at 120° C. for 18 h. The cooled reaction mixture was cooled with saturated aqueous NaHCO₃ solution and extracted with DCM (3×). The combined organic extracts was adsorbed on silica. The residue was purified by silica gel chromatography eluting with a gradient of EtOAc (0 to 100%) in heptane to provide 3-bromo-N-(2,2,2-trifluoroethyl)pyridin-2-amine (390 mg, 2.38 mmol, 90% yield). MS: [M+H]+: 255.0/257.0.

Step 2. To a MW vial charged with bis(pinacolato)diboron (602 mg, 2.37 mmol), 3-bromo-N-(2,2,2-trifluoroethyl)pyridin-2-amine (503 mg, 1.97 mmol), potassium acetate (600 mg, 6.11 mmol) and Pd(dppf)Cl₂·DCM (166 mg, 203.3 μmol) was added dioxane (8 mL). The solution was bubbled through with N2, the vial was capped and stirred at 100° C. for 2.5 h. The cooled reaction mixture was adsorbed on silica using DCM. The residue was purified by silica gel chromatography eluting with a gradient of EtOAc (0 to 60%) in heptane to provide 3-(4,4,5,5-tetramethyl-1,3,2-dioxaborolan-2-yl)-N-(2,2,2-trifluoroethyl)pyridin-2-amine (260 mg, 44% yield). MS: [M+H]+: 304.0

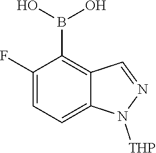

### Intermediate BX ((5-fluoro-1-(tetrahydro-2H-pyran-2-yl)-1H-indazol-4-yl)boronic acid)

Step 1. To a suspension of 4-bromo-5-fluoro-1H-indazole (2.0 g, 9.30 mmol) in MeCN (30 mL) was added para-toluene sulfonic acid (160.17 mg, 930.1 μmol) then 3,4-dihydropyran (2.55 mL, 28.1 mmol). The resulting solution was stirred at rt for 45 min. The mixture was concentrated, partitioned between saturated aqueous NaHCO₃ solution and EtOAc. The layers were separated and the aqueous layer was extracted with EtOAc (2×). The combined organic extracts was washed with brine, dried over Na₂SO₄, filtered and concentrated. The residue was purified by silica gel chromatography eluting with a gradient of EtOAc (0 to 50%) in DCM to provide 4-bromo-5-fluoro-1-tetrahydropyran-2-yl-indazole (1.66 g, 60% yield). MS: [M+H]+: 214.9/216.9.

Step 2. To a solution of 4-bromo-5-fluoro-1-tetrahydropyran-2-yl-indazole (1.66 g, 5.55 mmol) in THF (16 mL) in a dry-ice/acetone bath was added n-butyllithium solution 2.5 M in hexanes (3.4 mL, 8.5 mmol) dropwise. The mixture was stirred for 55 min at −78° C. then trimethylborate (1.89 mL, 16.65 mmol) was added dropwise. The mixture was stirred for 90 min at −78° C. then quenched with saturated aqueous solution of NH₄Cl. The mixture was extracted with EtOAc (3×). The combined organic extracts was washed with brine, dried over Na₂SO₄, filtered and concentrated. The residue was purified by silica gel chromatography eluting with a gradient of EtOAc (0 to 100%) in heptane to provide (5-fluoro-1-tetrahydropyran-2-yl-indazol-4-yl)boronic acid (756 mg, 52% yield). MS: [M+H]+: 265.0.

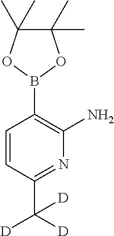 [SMILES: [2H]C([2H])([2H])c1ccc(B2OC(C)(C)C(C)(C)O2)c(N)n1]

### Intermediate BY (6-(methyl-d3)-3-(4,4,5,5-tetramethyl-1,3,2-dioxaborolan-2-yl)pyridin-2-amine)

Step 3. A. To a suspension of magnesium (675 mg, 27.8 mmol) in ether (27.50 mL) was added molecular iodine (60 mg, 236.4 μmol). The mixture was stirred at rt for 10 min and then to it was added trideuterio(iodo)methane (1.8 mL, 28.93 mmol) dropwise. After some drops of iodomethe-d3, the mixture was sonicated for 5 min. The color of the reaction changed from orange to yellow, then milky and finally cloudy metallic (high exotherm observed, the suspension was refluxed without any external heat). After the addition of iodomethane-d3, the mixture stirred at rt for 1 h 30. This solution was added to 0.5 M solution of ZnCl2 in THF (55 mL, 27.5 mmol) at rt dropwise. After addition, the mixture was stirred at rt for 20 min. This solution was added to a mixture of 3-bromo-6-iodo-pyridin-2-amine (1500 mg, 5.02 mmol) and Pd(PPh₃)₄ (576 mg, 498.5 μmol). The mixture was stirred at 60° C. for 1 h under N₂. The reaction was quenched with diluted aqueous HCl and water and extracted with EtOAc (2×50 mL). The combined organic extracts was washed with water and brine, dried over sodium sulfate, filtered and concentrated. The residue was purified by silica gel chromatography eluting with a gradient of EtOAc (0 to 60%) in heptane to provide 3-bromo-6-(trideuteriomethyl)pyridin-2-amine (600 mg, 63% yield. ¹H NMR (400 MHz, Chloroform-d) δ 7.47 (d, J=7.8 Hz, 1H), 6.36 (d, J=7.9 Hz, 1H), 4.95 (s, 2H).

Step 4. To a solution of 3-bromo-6-(trideuteriomethyl)pyridin-2-amine (272 mg, 1.43 mmol) in dioxane (10 mL) were added potassium acetate (354 mg, 3.61 mmol), Pd(dppf)Cl₂ (65 mg, 88.8 μmol) and bis(pinacolato)diboron (460 mg, 1.81 mmol). The mixture was degassed in vacuo, back-filled with N₂ and stirred at 110° C. under N₂ for 1 h to provide a solution of 6-(methyl-d3)-3-(4,4,5,5-tetramethyl-1,3,2-dioxaborolan-2-yl)pyridin-2-amine which was used as is for arylation.

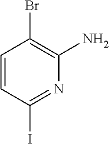

### Intermediate BZ (3-bromo-6-iodopyridin-2-amine)

A MW vessel containing 3-bromo-2-fluoro-6-iodo-pyridine (2.0 g, 6.63 mmol) and 7 N ammonia solution in MeOH (5 mL, 35 mmol) was sealed and stirred at 60° C. overnight. The solvent was evaporated and the residue was taken into EtOAc, washed with saturated aqueous solution of NaHCO₃ then with brine, dried over MgSO₄, filtered and concentrated. The residue was purified by silica gel chromatography eluting with a gradient of EtOAc (0 to 20%) in heptane to provide 3-bromo-6-iodo-pyridin-2-amine (1.32 g, 67% yield). MS: [M+H]+: 298.8/300.8.

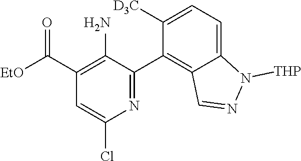

### Intermediate CA (ethyl 3-amino-6-chloro-2-(5-(methyl-d3)-1-(tetrahydro-2H-pyran-2-yl)-1H-indazol-4-yl)isonicotinate)

A RBF containing Intermediate AE (19.71 g, 57.09 mmol) was charged with ethyl 5-amino-2,6-dichloro-pyrimidine-4-carboxylate (13.0 g, 55.07 mmol), dioxane (280 mL), 2 M aqueous solution of K₃PO₄ (44 mL, 88 mmol) and Pd(dppf)Cl₂·DCM (4.50 g, 5.51 mmol). The mixture was bubbled with N₂ for 10 min then fitted with a condenser. 3 cycles of vacuum/nitrogen were done and the mixture was stirred at 85° C. for 3 h. The cooled reaction mixture was concentrated to a syrup and the material was diluted with EtOAc and water and this mixture was filtered through Celite™. The aqueous layer was cut from the filtrate and then extracted twice with EtOAc. The combined organic layer was washed with brine, dried over MgSO₄, filtered and concentrated. The residue was purified by silica gel chromatography eluting with a gradient of EtOAc (10 to 60%) in heptane to provide ethyl 5-amino-2-chloro-6-[1-tetrahydropyran-2-yl-5-(trideuteriomethyl)indazol-4-yl]pyrimidine-4-carboxylate (13.7 g, 59% yield). MS: [M+H]+: 419.0/420.9.

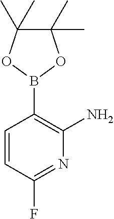 [SMILES: CC1(C)OB(c2ccc(F)nc2N)OC1(C)C]

### Intermediate CB (6-fluoro-3-(4,4,5,5-tetramethyl-1,3,2-dioxaborolan-2-yl)pyridin-2-amine)

A RBF was charged with Intermediate AQ (22.5 g, 117.8 mmol), XPhos (555 mg, 1.16 mmol), bis(pinacolato)diboron (35 g, 137.8 mmol) and potassium 2-ethylhexanoate (43.2 g, 237.0 mmol). Isopropyl acetate (650 mL) was added, then nitrogen was bubbled for 5 min and kept under a positive pressure of nitrogen. The thick suspension was stirred at 50° C. for 5-10 min then XPhos Pd(allyl)Cl (776 mg, 1.18 mmol) was added. The suspension was stirred under nitrogen at 50° C. for 4 hrs. The cooled reaction mixture was diluted with water and EtOAc. The layers were separated, the organic layer was washed sequentially with water and brine, dried over Na₂SO₄, filtered and adsorbed on silica. The residue was purified by silica gel chromatography eluting with a gradient of EtOAc (0 to 30%) in heptane to provide 6-fluoro-3-(4,4,5,5-tetramethyl-1,3,2-dioxaborolan-2-yl)pyridin-2-amine (22.2 g, 79% yield). MS: [M+H]+: 239.3.

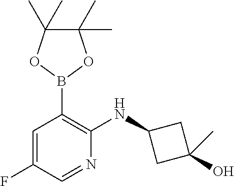 [SMILES: CC1(C)OB(c2cc(F)cnc2N[C@H]2C[C@@](C)(O)C2)OC1(C)C]

### Intermediate CD ((1s,3s)-3-((5-fluoro-3-(4,4,5,5-tetramethyl-1,3,2-dioxaborolan-2-yl)pyridin-2-yl)amino)-1-methylcyclobutan-1-ol)

Step 1. To a solution of 3-bromo-2,5-difluoro-pyridine (4.5 g, 23.2 mmol) and K₂CO₃ (8.10 g, 58.6 mmol) in DMSO (20 mL) was added (1s,3s)-3-amino-1-methylcyclobutan-1-ol hydrochloride (3.30 g, 24.0 mmoll) at rt. The mixture was stirred at 100° C. for 18 h. The cooled reaction mixture was poured into water and extracted with ether. The organic layer was separated, washed with water and brine, dried over MgSO₄ and concentrated. The residue was purified by silica gel chromatography eluting with a gradient of EtOAc (0 to 65%) in heptane to provide (1s,3s)-3-((3-bromo-5-fluoropyridin-2-yl)amino)-1-methylcyclobutan-1-ol (5.8 g, 91% yield). MS: [M+H]+: 275.0/277.0.

Step 2. An oven dried 3-neck RBF with a condenser was charged with (1s,3s)-3-((3-bromo-5-fluoropyridin-2-yl)amino)-1-methylcyclobutan-1-ol (39.8 g, 144.7 mmol), bis(pinacolato)diboron (47.55 g, 187.3 mmol) and potassium acetate (35.5 g, 361.7 mmol). Dioxane (600 mL) was added and the mixture was bubbled through with N₂. Pd(dppf)Cl₂·DCM (4.87 g, 5.96 mmol) was added, the mixture was bubbled through with N₂ and stirred at 110° C. for 90 min. The cooled reaction mixture was diluted with water (600 mL) and filtered on Celite™, washing with EtOAc (600 mL total). The layers were separated and the aqueous layer was extracted with EtOAc (300 mL). The combined organic extracts was washed with brine, dried over Na₂SO₄, filtered and concentrated. The residue was purified by silica gel chromatography eluting with a gradient of EtOAc (25 to 100%) in heptane. The solid was triturated in heptane at 50° C. to provide (1s,3s)-3-((5-fluoro-3-(4,4,5,5-tetramethyl-1,3,2-dioxaborolan-2-yl)pyridin-2-yl)amino)-1-methylcyclobutan-1-ol (25.09 g, 54% yield). MS: [M+H]+: 323.3. ¹H NMR (400 MHz, Chloroform-d) δ 8.00 (dd, J=3.3, 0.8 Hz, 1H), 7.56 (dd, J=8.3, 3.2 Hz, 1H), 6.25 (d, J=6.8 Hz, 1H), 4.03 (h, J=7.7 Hz, 1H), 2.62 (ddt, J=9.5, 7.6, 2.4 Hz, 2H), 2.13 (s, 1H), 2.06-1.97 (m, 2H), 1.44 (s, 3H), 1.34 (s, 12H).

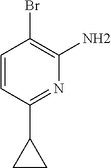 [SMILES: Nc1nc(C2CC2)ccc1Br]

### Intermediate CE (3-bromo-6-cyclopropylpyridin-2-amine)

To a mixture of Intermediate BZ (1.0 g, 3.35 mmol) and Pd(PPh₃)₄ (384 mg, 332.3 μmol) in THF (20 mL) was added 0.5 M solution of cyclopropylzinc bromide in THF (26 mL, 13 mmol) dropwise. The mixture was stirred at 60° C. for 1 h under N₂. The cooled reaction mixture was quenched with saturated aqueous NH₄Cl solution and extracted with EtOAc (2×50 mL). The combined organic extracts were washed with brine, dried over sodium sulfate, filtered and concentrated. The residue was purified by silica gel chromatography eluting with a gradient of EtOAc (0 to 40%) in heptane to provide 3-bromo-6-cyclopropyl-pyridin-2-amine (460 mg, 65% yield). MS: [M+H]+: 213.0/215.0. ¹H NMR (400 MHz, Chloroform-d) δ 7.43 (d, J=7.9 Hz, 1H), 6.37 (d, J=7.9 Hz, 1H), 4.74 (s, 2H), 1.90-1.71 (m, 1H), 0.96-0.80 (m, 4H).

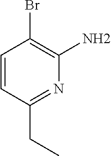

### Intermediate CF (3-bromo-6-ethylpyridin-2-amine)

In a flame dried RBF, 3-bromo-6-iodopyridin-2-amine (400 mg, 1.34 mmol) was dissolved in THF (6.7 mL). The mixture was degassed with nitrogen for 10 minutes and Pd(PPh₃)₄ (156 mg, 134 μmol) was added at rt, following by the drop wise addition of 2 M diethylzinc solution hexanes (1.34 mL, 2.68 mmol). The mixture was stirred at 60° C. for 1 h under nitrogen. The reaction was quenched with saturated aqueous solution of NH₄Cl and extracted with EtOAc (2×50 mL). The combined organic extracts were washed with brine, dried over sodium sulfate, filtered and concentrated. The residue was purified by silica gel chromatography eluting with a gradient of EtOAc (0 to 40%) in heptane to provide 3-bromo-6-ethylpyridin-2-amine (53 mg, 20%). MS: [M+H]+: 201.0/203.0.

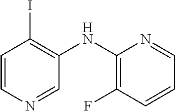 [SMILES: Fc1cccnc1Nc1cnccc1I]

### Intermediate CG (3-fluoro-N-(4-iodopyridin-3-yl)pyridin-2-amine)

2,3-Difluoropyridine (212 mg, 1.77 mmol) and 3-amino-4-iodopyridine (400 mg, 1.82 mmol) were dissolved in DMF (6.2 mL). Sodium hydride (60% dispersion in mineral oil, 197 mg, 4.91 mmol) was added portion wise at rt and the mixture was stirred at rt for 1 h. MeOH (1 mL) and silica were added and the mixture was concentrated. The residue was purified by silica gel chromatography eluting with a gradient of EtOAc (5 to 100%) in heptane to provide 3-fluoro-N-(4-iodopyridin-3-yl)pyridin-2-amine (300 mg, 78% yield). MS: [M+H]+: 315.9.

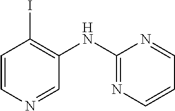 [SMILES: Ic1ccncc1Nc1ncccn1]

### Intermediate CH (N-(4-iodopyridin-3-yl)pyrimidin-2-amine)

2-Chloropyrimidine (187 μL, 1.81 mmol) and 3-amino-4-iodopyridine (400 mg, 1.82 mmol) were dissolved in DMF (6.2 mL). Sodium hydride (60% dispersion in mineral oil, 197 mg, 4.91 mmol) was added portion wise at rt and the mixture was stirred at rt for 1 h. MeOH (1 mL) and silica were added and the mixture was concentrated. The residue was purified by silica gel chromatography eluting with a gradient of EtOAc (5 to 100%) in heptane to provide N-(4-iodopyridin-3-yl)pyrimidin-2-amine (270 mg, 74% yield). MS: [M+H]+: 299.3.

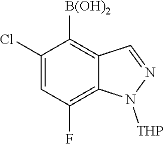

### Intermediate CI ((5-chloro-7-fluoro-1-(tetrahydro-2H-pyran-2-yl)-1H-indazol-4-yl)boronic acid)

Step 1. 4-Bromo-7-fluoro-1H-indazole (1.2 g, 5.47 mmol) was dissolved in concentrated sulfuric acid (7.5 mL) and cooled in an ice bath with stirring. Concentrated nitric acid (2.4 mL) was added dropwise and the reaction was stirred 1 h. The reaction was poured over ice-water and the resulting precipitate was collected by vacuum filtration and suction-dried affording 4-bromo-7-fluoro-5-nitro-1H-indazole (1.22 g 86% yield). MS: [M+H]+: 260.0.

Step 2. 4-Bromo-7-fluoro-5-nitro-1H-indazole (1.2 g, 4.62 mmol) and ammonium chloride (1.48 g, 27.7 mmol) were stirred in a mixture of water (10 mL), methanol (10 mL) and THF (10 mL). Iron powder (1.03 g, 18.5 mmol) was added and the reaction was heated to 80° C. for 1 h with stirring. The reaction was cooled to ambient temperature and filtered through Celite™ with EtOAc as eluent. The filtrate was adsorbed onto silica gel and the residue was purified by silica gel chromatography eluting with a gradient of EtOAc (2 to 80%) in heptane to provide 4-bromo-7-fluoro-1H-indazol-5-amine (720 mg, 68% yield). MS: [M+H]+: 231.9.

Step 3. 4-Bromo-7-fluoro-1H-indazol-5-amine (1.0 g, 4.35 mmol) was sonicated in concentrated HCl (7 mL) until dissolved then cooled in an ice bath with stirring. To this stirring solution was added a solution of sodium nitrite (360 mg, 522 mmol) in water (7 mL). The mixture was stirred for 15 mins at 0° C. and then slowly added to a stirring suspension of copper (I) chloride (887 mg, 8.69 mmol) in water (20 mL) at 60° C. Once addition was complete the reaction was allowed to stir at 60° C. an additional 30 min and then cooled in an ice bath. Potassium carbonate was added slowly with stirring until gas evolution ceased. Ammonium hydroxide (28-30%, 10 mL, 257 mmol) was then added and the product was extracted into EtOAc. The organic layer was washed with water and brine, dried over sodium sulfate, filtered and adsorbed onto silica gel. The residue was purified by silica gel chromatography eluting with a gradient of EtOAc (2 to 80%) in heptane to provide 4-bromo-5-chloro-7-fluoro-1H-indazole (730 mg, 67% yield). MS: [M+H]+: 251.3.

Step 4. A solution of 3,4-dihydro-2H-pyran (550 μL, 5.85 mmol) and p-toluenesulfonic acid monohydrate (56.5 mg, 293 μmol) in DCM (1.5 mL) was added to a stirring solution of 4-bromo-5-chloro-7-fluoro-1H-indazole (730 mg, 2.93 mmol) in DCM (7.6 mL). The mixture was stirred overnight at room temperature and adsorbed directly onto silica gel. The residue was purified by silica gel chromatography eluting with a gradient of EtOAc (2 to 60%) in heptane to provide 4-bromo-5-chloro-7-fluoro-1-(tetrahydro-2H-pyran-2-yl)-1H-indazole (480 mg, 49% yield). MS: [M−THP+H]+: 251.3.

Step 5. A stirring solution of 4-bromo-5-chloro-7-fluoro-1-(tetrahydro-2H-pyran-2-yl)-1H-indazole (350 mg, 1.05 mmol) and triisopropyl borate (737 μL, 3.15 mmol) in dry THF (6.00 mL) was cooled to −78° C. under nitrogen. tert-Butyllithium 1.7 M in pentane (1.23 mL, 2.10 mmol) was added dropwise. The mixture was stirred at −78° C. for 30 min. Another portion of tert-Butyllithium 1.7 M in pentane (1.23 mL, 2.10 mmol) was added and the mixture was stirred at −78° C. for 30 more min. Again, another portion of tert-Butyllithium 1.7 M in pentane (1.23 mL, 2.10 mmol) was added and the mixture was stirred at −78° C. for 30 min. The reaction was quenched with saturated aqueous ammonium chloride and extracted with EtOAc, combined organics were washed with brine, dried over sodium sulfate, filtered and adsorbed onto silica gel. The residue was purified by silica gel chromatography eluting with a gradient of i-PrOH (1 to 40%) in DCM to provide (5-chloro-7-fluoro-1-(tetrahydro-2H-pyran-2-yl)-1H-indazol-4-yl)boronic acid (125 mg, 40% yield). MS: [M−THP+H]+: 215.2.

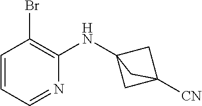 [SMILES: N#CC12CC(Nc3ncccc3Br)(C1)C2]

### Intermediate CM (3-((3-bromopyridin-2-yl)amino)bicyclo[1.1.1]pentane-1-carbonitrile)

To a solution of 3-bromo-2-iodo-pyridine (400 mg, 1.41 mmol) in toluene (15 mL) were added 3-aminobicyclo[1.1.1]pentane-1-carbonitrile hydrochloride (230 mg, 1.59 mmol), Pd(OAc)₂ (30 mg, 133.6 μmol), cesium carbonate (1.1 g, 3.38 mmol) and rac-BINAP (172 mg, 276.2 μmol). The mixture was degassed in vacuo, back-filled with N₂ and stirred at 110° C. for 2 h. The cooled reaction mixture was concentrated and the residue was purified by silica gel chromatography eluting with a gradient of EtOAc (0 to 50%) in heptane to provide 3-[(3-bromo-2-pyridyl)amino]bicyclo[1.1.1]pentane-1-carbonitrile (115 mg, 31% yield). ¹H NMR (400 MHz, Chloroform-d) δ 8.03 (dd, J=4.8, 1.6 Hz, 1H), 7.58 (dd, J=7.7, 1.6 Hz, 1H), 6.52 (dd, J=7.7, 4.8 Hz, 1H), 2.63 (s, 6H).

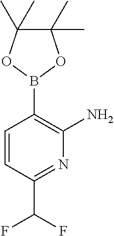 [SMILES: CC1(C)OB(c2ccc(C(F)F)nc2N)OC1(C)C]

### Intermediate CN (6-(difluoromethyl)-3-(4,4,5,5-tetramethyl-1,3,2-dioxaborolan-2-yl)pyridin-2-amine)

To a solution of Intermediate BL (256 mg, 1.15 mmol) in dioxane (10 mL) were added potassium acetate (283 mg, 2.88 mmol), Pd(dppf)Cl₂ (55 mg, 75.2 μmol) and bis(pinacolato)diboron (370 mg, 1.46 mmol). The mixture was degassed in vacuo, back-filled with N₂ and stirred at 110° C. under N₂ for 1 h to provide a solution of 6-(difluoromethyl)-3-(4,4,5,5-tetramethyl-1,3,2-dioxaborolan-2-yl)pyridin-2-amine which was used as is for arylation.

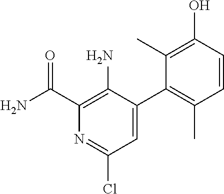 [SMILES: Cc1ccc(O)c(C)c1-c1cc(Cl)nc(C(N)=O)c1N]

### Intermediate CO (3-amino-6-chloro-4-(3-hydroxy-2,6-dimethylphenyl)picolinamide)

Step 1. To a solution of Intermediate D (6.0 g, 21.5 mmol) in toluene (100 mL) were added 2-(3-methoxy-2,6-dimethyl-phenyl)-4,4,5,5-tetramethyl-1,3,2-dioxaborolane (6.00 g, 22.9 mmol), 2 M aqueous solution of Na₂CO₃ (21 mL, 42 mmol), tri-tert-butylphosphine 1 M in toluene (4.2 mL, 4.20 mmol) and Pd₂(dba)₃ (1.95 g, 2.13 mmol). The mixture was degassed in vacuo and then back-filled with N₂ then stirred at 90° C. for 2 h. The volatiles were removed in vacuo and the residue was purified using flash silica gel chromatography eluting with EtOAc/hexanes 0-30% to provide ethyl 3-amino-6-chloro-4-(3-methoxy-2,6-dimethyl-phenyl)pyridine-2-carboxylate (4.56 g, 63% yield). MS: [M+H]⁺: 335.2.

Step 2. A 150 mL pressure vessel was charged with ethyl 3-amino-6-chloro-4-(3-methoxy-2,6-dimethyl-phenyl)pyridine-2-carboxylate (1.45 g, 4.32 mmol) and 7 N ammonia solution in MeOH (50 mL, 350 mmol). The vessel was sealed and stirred at 70° C. overnight. The mixture was cooled to rt then concentrated to dryness. The residue was purified by flash silica gel chromatography eluting with EtOAc/hexanes 0-50% to provide 3-amino-6-chloro-4-(3-methoxy-2,6-dimethyl-phenyl)pyridine-2-carboxamide (1.13 g, 86% yield). MS: [M+H]⁺: 306.2.

Step 3. To a solution of 3-amino-6-chloro-4-(3-methoxy-2,6-dimethyl-phenyl)pyridine-2-carboxamide (950 mg, 3.11 mmol) in DCM (12 mL) was added 1 M BBr₃ solution in DCM (9.3 mL, 9.3 mmol). The mixture was stirred at rt for 20 min. Silica was added to the mixture and the volatiles were removed in vacuo. The residue was purified by silica gel chromatography eluting with a gradient of MeOH (0 to 10%) in DCM to provide 3-amino-6-chloro-4-(3-hydroxy-2,6-dimethyl-phenyl)pyridine-2-carboxamide (906 mg, 99% yield). MS: [M+H]⁺: 292.2.

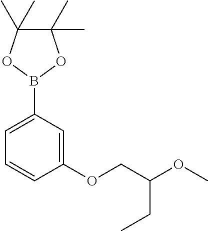

### Intermediate CP (2-(3-(2-methoxybutoxy)phenyl)-4,4,5,5-tetramethyl-1,3,2-dioxaborolane)

Step 1. A solution of 2-methoxybutan-1-ol (0.2 g, 1.92 mmol) in dichloromethane (5 mL) was added Et₃N (0.389 g, 3.846 mmol) at 0° C. The mixture was stirred for 10 min and methanesulfonyl chloride (0.262 g, 2.3 mmol) was added. The mixture was stirred at rt for 2 h. The reaction mixture was poured in to water (10 mL) and extracted with ethyl acetate (3×10 mL). The combined organic layer was dried over sodium sulfate and concentrated to provide 2-methoxybutyl methanesulfonate (0.320 g, 91% yield) which was used in next step without purification. ¹H NMR (400 MHz, DMSO-d6) δ 4.35-4.18 (m, 2H), 3.47 (s, 3H), 3.42-3.40 (m, 1s), 3.1 (s, 3H), 1.66-1.59 (m, 2H), 1.01-0.99 (m, 3H).

Step 2. To the stirred solution of 2-methoxybutyl methanesulfonate (0.32 g, 1.75 mmol) in DMF (3.5 ml) was added K₂CO₃ (0.727 g, 5.27 mmol) and 3-bromophenol (0.272 g, 1.575 mmol). The resulting mixture was heated at 80° C. for 16 h. The cooled reaction mixture was poured into water (10 mL) and extracted with ethyl acetate (3×10 mL). The combined organic layer was dried over sodium sulfate and concentrated. The residue was purified by silica gel chromatography eluting with a gradient of EtOAc (0 to %) in hexanes to provide 1-bromo-3-(2-methoxybutoxy) benzene (0.4 g, 83%). ¹H NMR (400 MHz, DMSO-d6) δ 7.19-7.11 (m, 3H), 6.89 (d, J=8 Hz, 1H), 3.99 (d, J=4.8 Hz, 2H), 3.51 (s, 4H), 1.75-1.62 (m, 2H), 1.02 (t, J=7.6 Hz, 3H).

Step 3. A solution of 1-bromo-3-(2-methoxybutoxy) benzene (0.4 g, 1.54 mmol), bis(pinacolato)diboron (0.588 g, 2.31 mmol) and KOAc (0.452 g, 4.62 mmol) in dioxane (10 mL) was purged with N2 gas for 15 min. PdCl₂(dppf)·DCM (0.125 g, 0.154 mmol) was added and the mixture was stirred at 100° C. for 5 h. The cooled reaction mixture was poured into water and extracted with ethyl acetate (3×10 mL). The combined organic layer was dried over sodium sulfate and concentrated to give 2-(3-(2-methoxybutoxy)phenyl)-4,4,5,5-tetramethyl-1,3,2-dioxaborolane (0.3 g, 63%), which was used for next step without purification. MS: [M+H]⁺: 307.2.

### Compounds

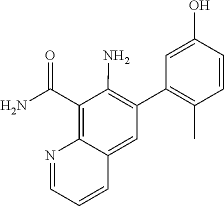 [SMILES: Cc1ccc(O)cc1-c1cc2cccnc2c(C(N)=O)c1N]

### Compound 1 (7-amino-6-(5-hydroxy-2-methylphenyl)quinoline-8-carboxamide)

Step 1. A mixture of [5-(methoxymethoxy)-2-methyl-phenyl]boronic acid (13 mg, 66 μmol), potassium carbonate (29 mg, 211 μmol), Pd(PPh₃)₄ (7.0 mg, 6 μmol) and Intermediate A (15 mg, 60 μmol) in toluene and water (0.5 mL) was degassed and stirred under reflux for 5 h. The solvents were removed under reduced pressure and the residue was purified by preparative HPLC eluting with ACN/water/0.1% formic acid to provide 7-amino-6-[5-(methoxymethoxy)-2-methyl-phenyl]quinoline-8-carbonitrile (11 mg, 57% yield). MS: [M+H]⁺: 320.2

Step 2. To a suspension of 7-amino-6-[5-(methoxymethoxy)-2-methyl-phenyl]quinoline-8-carbonitrile (10.0 mg, 31 μmol) in DCM (1 mL) was added 4 M HCl solution in dioxane (47 μL, 188 μmol). The mixture was stirred at rt for 3 h. The solvent was removed in vacuo to provide 7-amino-6-(5-hydroxy-2-methyl-phenyl)quinoline-8-carbonitrile (9 mg, 99% yield). MS: [M+H]⁺: 276.1.

Step 3. To a microwave vessel was containing 7-amino-6-(5-hydroxy-2-methyl-phenyl)quinoline-8-carbonitrile (9 mg, 33 μmol) in methanol (500 μL) was added 4 M solution of NaOH (100 μL, 400 μmol).

The vessel was sealed and the mixture was heated at 130° C. for 30 minutes. The reaction mixture was purified by preparative HPLC eluting with ACN/water/0.1% formic acid to provide 7-amino-6-(5-hydroxy-2-methyl-phenyl)quinoline-8-carboxamide (0.4 mg, 4% yield). MS: [M+H]⁺: 294.1; ¹H NMR (400 MHz, Chloroform-d) δ 8.67 (d, J=2.4 Hz, 1H), 7.88 (d, J=2.4 Hz, 1H), 7.58 (d, J=9.0 Hz, 1H), 7.16 (d, J=8.1 Hz, 2H), 6.91 (d, J=8.9 Hz, 1H), 6.83-6.69 (m, 3H), 5.88 (s, 2H), 2.21 (s, 3H).

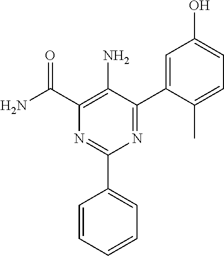 [SMILES: Cc1ccc(O)cc1-c1nc(-c2ccccc2)nc(C(N)=O)c1N]

### Compound 2 (5-amino-6-(5-hydroxy-2-methylphenyl)-2-phenylpyrimidine-4-carboxamide)

Step 1. To a solution of Intermediate C (26 mg, 80.56 μmol) in dioxane (2 mL) were added sodium carbonate (2 M, 160 μL), phenylboronic acid (18 mg, 147.6 μmol) and Pd(dppf)Cl₂ (6 mg, 8.2 μmol). The mixture was degassed in vacuo, back-filled with N₂ and then stirred at 120° C. for 5 h. The volatiles were removed in vacuo and the residue was purified using flash silica gel chromatography silica gel eluting with EtOAc/hexanes 0-50% to provide 5-amino-6-[5-(methoxymethoxy)-2-methyl-phenyl]-2-phenyl-pyrimidine-4-carboxamide (18 mg, 49.40 μmol, 61.32% yield). MS: [M+H]⁺: 365.2; ¹H NMR (400 MHz, Chloroform-d) δ 8.42-8.24 (m, 2H), 8.11 (d, J=4.5 Hz, 1H), 7.45-7.31 (m, 3H), 7.31-7.21 (m, 1H), 7.12-7.00 (m, 2H), 5.94 (s, 2H), 5.75 (d, J=4.6 Hz, 1H), 5.16 (s, 2H), 3.47 (s, 3H), 2.17 (s, 3H).

Step 2. To a solution of 5-amino-6-[5-(methoxymethoxy)-2-methyl-phenyl]-2-phenyl-pyrimidine-4-carboxamide (18 mg, 49.4 μmol) in DCM (1 mL) was added h4 M HCl solution in dioxane (200 μL, 0.8 mmol). The mixture was stirred at rt for 1 h and the volatiles were removed in vacuo. The residue was dissolved in water and acetonitrile then lyophilized to provide 5-amino-6-(5-hydroxy-2-methylphenyl)-2-phenylpyrimidine-4-carboxamide hydrochloride (17 mg, 96% yield). MS: [M+H]⁺: 321.2; ¹H NMR (400 MHz, DMSO-d6) δ 9.57 (s, 1H), 8.50 (s, 1H), 8.44-8.29 (m, 2H), 7.79 (s, 1H), 7.52-7.31 (m, 3H), 7.14 (d, J=8.4 Hz, 1H), 6.78 (dd, J=8.3, 2.6 Hz, 1H), 6.69 (d, J=2.6 Hz, 1H), 6.49 (s, 2H), 2.00 (s, 3H).

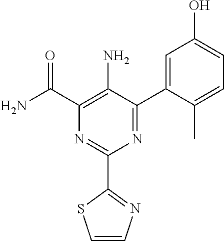 [SMILES: Cc1ccc(O)cc1-c1nc(-c2nccs2)nc(C(N)=O)c1N]

### Compound 3 (5-amino-6-(5-hydroxy-2-methylphenyl)-2-(thiazol-2-yl)pyrimidine-4-carboxamide)

Step 1. A solution of tributyl(thiazol-2-yl)stannane (140 μL, 445 μmol), Intermediate C (70 mg, 217 μmol), copper(I) iodide (5 mg, 26 μmol), lithium chloride (13 mg, 307 μmol) and Pd(dppf)Cl₂ (16 mg, 22 μmol) in DMF (2 mL) was degassed in vacuo and then back-filled with N2. It was then stirred at 110° C. for 2 h. Then the volatiles were removed in vacuo and the residue was purified using flash silica gel chromatography eluting with MeOH/DCM 0-10% to provide 5-amino-6-[5-(methoxymethoxy)-2-methyl-phenyl]-2-thiazol-2-yl-pyrimidine-4-carboxamide (52 mg, 65% yield). MS: [M+H]⁺: 372.2.

Step 2. To a solution of 5-amino-6-[5-(methoxymethoxy)-2-methyl-phenyl]-2-thiazol-2-yl-pyrimidine-4-carboxamide (52 mg, 140.0 μmol) in DCM (1 mL) were added 4 M HCl solution in dioxane (0.35 mL, 1.4 mmol). The mixture was stirred at rt for 1 h. The volatiles were removed in vacuo and the residue was purified by preparative HPLC C18 column eluting with ACN/water/0.1% formic acid to provide 5-amino-6-(5-hydroxy-2-methyl-phenyl)-2-thiazol-2-yl-pyrimidine-4-carboxamide (25 mg, 55% yield). MS: [M+H]⁺: 328.2; ¹H NMR (400 MHz, DMSO-d6) δ 9.47 (s, 1H), 8.11 (d, J=2.6 Hz, 1H), 7.90 (dd, J=6.9, 2.9 Hz, 2H), 7.76 (d, J=3.2 Hz, 1H), 7.15 (d, J=8.3 Hz, 1H), 6.93-6.47 (m, 4H), 2.00 (s, 3H).

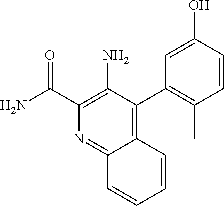 [SMILES: Cc1ccc(O)cc1-c1c(N)c(C(N)=O)nc2ccccc12]

### Compound 10 (3-amino-4-(5-hydroxy-2-methylphenyl)quinoline-2-carboxamide)

Step 1. A suspension of ethyl 4-oxo-1H-quinoline-2-carboxylate (1.07 g, 4.92 mmol) and NIS (1.22 g, 5.41 mmol) in ACN (10 mL) and acid acetic (0.5 mL) was stirred at 90° C. for 17 h. The solid was filtered to provide ethyl 4-hydroxy-3-iodo-quinoline-2-carboxylate (962 mg, 57% yield). MS: [M+H]⁺: 344.0.

Step 2. To ethyl 4-hydroxy-3-iodo-quinoline-2-carboxylate (800 mg, 2.33 mmol) in pyridine (5 mL) at 0° C. was added 1 M solution of trifluoromethylsulfonyl trifluoromethanesulfonate in DCM (3.5 mL, 3.5 mmol) and the mixture was stirred at rt for 17 h. The solvents were removed under reduced pressure and the residue was purified by silica gel chromatography eluting with a gradient of EtOAc (0 to 100%) in heptane to provide ethyl 3-iodo-4-(trifluoromethylsulfonyloxy)quinoline-2-carboxylate (1.0 g, 90% yield). MS: [M+H]⁺: 476.1.

Step 3. [5-(methoxymethoxy)-2-methyl-phenyl]boronic acid (453.7 mg, 2.31 mmol), K₂CO₃ (1.02 g, 7.37 mmol), Pd(PPh₃)₄ (243 mg, 210.5 μmol) and ethyl 3-iodo-4-(trifluoromethylsulfonyloxy)quinoline-2-carboxylate (1.0 g, 2.10 mmol) in toluene (10 mL) and water (2.5 mL) were degassed and stirred under reflux for 5 h. The solvents were moved under reduced pressure and the residue was purified by silica gel chromatography eluting with a gradient of EtOAc (0 to 100%) in heptane to provide ethyl 3-iodo-4-[5-(methoxymethoxy)-2-methyl-phenyl]quinoline-2-carboxylate (450 mg, 45% yield). MS: [M+H]⁺: 478.2.

Step 4. Iron(III) chloride (7.0 mg, 41.9 μmol) and copper(I) iodide (8.0 mg, 41.9 μmol) were added to a solution of ethyl 3-iodo-4-[5-(methoxymethoxy)-2-methyl-phenyl]quinoline-2-carboxylate (100 mg, 209.5 μmol) in EtOH (10 mL). 2 M solution of ammonia in EtOH (576 μL, 1.152 mmol) and sodium hydroxide (16.8 mg, 419.03 μmol) were successively added to the reaction mixture. The reaction tube was sealed and then heated at 90° C. for 16 h. The cooled reaction mixture was extracted with EtOAc and concentrated in vacuo to afford a crude mixture 3-amino-4-[5-(methoxymethoxy)-2-methyl-phenyl]quinoline-2-carboxamide (10.6 mg, 15% yield). MS: [M+H]⁺: 338.4.

Step 5. To the suspension of 3-amino-4-[5-(methoxymethoxy)-2-methyl-phenyl]quinoline-2-carboxamide (10.6 mg, 31.3 μmol) in DCM (1 mL) was added 4 M HCl solution in dioxane (47 μL, 188 μmol). The mixture was stirred at rt for 3 h. The volatiles were removed in vacuo and the residue was purified preparative HPLC C18 column eluting with ACN/water/0.1% formic acid to provide 3-amino-4-(5-hydroxy-2-methyl-phenyl)quinoline-2-carboxamide (4 mg, 44% yield). MS: [M+H]⁺: 294.3; ¹H NMR (400 MHz, Chloroform-d) δ 8.34 (s, 1H), 7.90 (ddd, J=8.4, 1.4, 0.7 Hz, 1H), 7.45-7.25 (m, 3H), 7.09 (ddd, J=8.4, 1.5, 0.7 Hz, 1H), 6.88 (dd, J=8.3, 2.8 Hz, 1H), 6.63 (d, J=2.7 Hz, 1H), 5.78 (s, 2H), 5.53 (s, 1H), 1.98 (s, 3H).

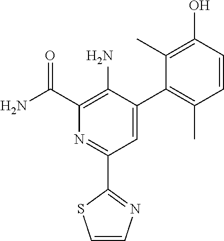 [SMILES: Cc1ccc(O)c(C)c1-c1cc(-c2nccs2)nc(C(N)=O)c1N]

### Compound 13 (3-amino-4-(3-hydroxy-2,6-dimethylphenyl)-6-(thiazol-2-yl)picolinamide)

Step 1. A solution of tributyl(thiazol-2-yl)stannane (119.00 mg, 318.04 μmol), Intermediate E (50 mg, 163.5 μmol), copper(I) iodide (4 mg, 21.0 μmol), lithium chloride (12 mg, 283.09 μmol), Pd(dppf)Cl₂ (13 mg, 17.8 μmol) in DMF (1 mL) was degassed in vacuo and then back-filled with N₂. The solution was then stirred at 110° C. for 1 h. The volatiles were removed in vacuo and the residue was purified using flash silica gel chromatography eluting with EtOAc/hexanes 0-50% to provide 3-amino-4-(3-methoxy-2,6-dimethyl-phenyl)-6-thiazol-2-yl-pyridine-2-carboxamide (35 mg, 60% yield). MS: [M+H]⁺: 355.2.

Step 2. To a solution of 3-amino-4-(3-methoxy-2,6-dimethyl-phenyl)-6-thiazol-2-yl-pyridine-2-carboxamide (35 mg, 98.75 μmol) in DCE (1 mL) was added 1 M solution of BBr₃ in DCM (300 μL, 300 μmol). The mixture was stirred at 45° C. for 0.5 h and the volatiles were removed in vacuo. The residue was treated with MeOH and concentrated to dryness again. The residue was purified by preparative HPLC C18 column eluting with ACN/water/0.1% formic acid to provide 3-amino-4-(3-hydroxy-2,6-dimethyl-phenyl)-6-thiazol-2-yl-pyridine-2-carboxamide (20 mg, 60% yield). MS: [M+H]⁺: 341.2; ¹H NMR (400 MHz, DMSO-d6) δ 9.31 (s, 1H), 7.86 (d, J=3.1 Hz, 1H), 7.80 (d, J=3.2 Hz, 1H), 7.69 (d, J=3.2 Hz, 1H), 7.66 (d, J=3.1 Hz, 1H), 7.62 (s, 1H), 7.10-6.84 (m, 1H), 6.78 (d, J=8.2 Hz, 1H), 6.52 (s, 2H), 1.84 s, 3H), 1.77 (s, 3H).

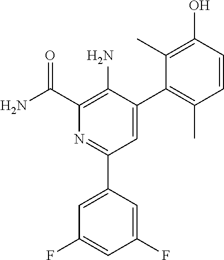 [SMILES: Cc1ccc(O)c(C)c1-c1cc(-c2cc(F)cc(F)c2)nc(C(N)=O)c1N]

### Compound 16 (3-amino-6-(3,5-difluorophenyl)-4-(3-hydroxy-2,6-dimethylphenyl)picolinamide)

Step 1. To a solution of Intermediate E (50 mg, 163.5 μmol) in dioxane (1 mL) were added 2 M aqueous Na₂CO₃ solution (200 μL, 400 μmol), (3,5-difluorophenyl)boronic acid (35 mg, 221.6 μmol), Pd(dppf)Cl₂ (10 mg, 13.7 μmol). The mixture was degassed in vacuo, back-filled with N₂ and then stirred at 120° C. for 1 h. The volatiles were removed in vacuo and the residue was purified using flash silica gel chromatography eluting with EtOAc/hexanes 20-100% to provide 3-amino-6-(3,5-difluorophenyl)-4-(3-methoxy-2,6-dimethyl-phenyl)pyridine-2-carboxamide (52 mg, 83% yield). as an off-white solid. MS: [M+H]⁺: 384.2.

Step 2. To a solution of 3-amino-6-(3,5-difluorophenyl)-4-(3-methoxy-2,6-dimethyl-phenyl)pyridine-2-carboxamide (52 mg, 135.63 μmol) in DCE (1.2 mL) was added 1 M BBr₃ solution in DCM (400 μL, 400 μmol). The mixture was stirred at 45° C. for 0.5 h. The volatiles were removed in vacuo and the residue was treated with MeOH and concentrated to dryness again. The residue was purified by preparative HPLC C18 column eluting with ACN/water 10 mM ammonium bicarbonate to provide 3-amino-6-(3,5-difluorophenyl)-4-(3-hydroxy-2,6-dimethyl-phenyl)pyridine-2-carboxamide (32 mg, 64% yield). MS: [M+H]⁺: 370.2; ¹H NMR (400 MHz, DMSO-d6) δ 9.26 (s, 1H), 8.40 (d, J=2.7 Hz, 1H), 8.07-7.82 (m, 2H), 7.72 (s, 1H), 7.48 (d, J=2.7 Hz, 1H), 7.07 (m, 1H), 6.96 (d, J=8.2 Hz, 1H), 6.77 (d, J=8.2 Hz, 1H), 6.15 (bs, 2H), 1.84 (s, 3H), 1.78 (s, 3H).

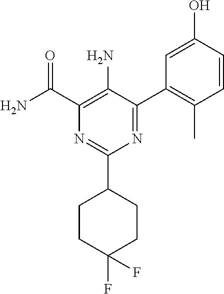 [SMILES: Cc1ccc(O)cc1-c1nc(C2CCC(F)(F)CC2)nc(C(N)=O)c1N]

### Compound 41 (5-amino-2-(4,4-difluorocyclohexyl)-6-(5-hydroxy-2-methylphenyl)pyrimidine-4-carboxamide)

Step 1. A MW vial was charged with Intermediate B (0.3 g, 0.925 mmol), dioxane-water 9:1 (5 mL), (4,4-difluorocyclohex-1-en-1-yl)boronic acid (0.31 g 1.85 mmol) and Cs₂CO₃ (0.9 g, 2.77 mmol). The reaction mixture was purged with N₂ gas for 10 min. XPhos-Pd-G3 (0.078 g, 0.092 mmol) was then added and the reaction mixture was heated at 140° C. for 2.5 h in MW. The cooled reaction mixture was diluted with water (20 mL) and extracted with EtOAc (3×20 mL). The combined organic layer was dried over Na₂SO₄, filtered and concentrated. The residue was purified by silica gel chromatography eluting with 20% ethyl acetate in hexanes to provide 5-amino-2-(4,4-difluorocyclohex-1-en-1-yl)-6-(5-(methoxymethoxy)-2-methylphenyl)pyrimidine-4-carboxamide (0.28 g, 64% yield). MS: [M+H]⁺: 405.3.

Step 2. 5-Amino-2-(4,4-difluorocyclohex-1-en-1-yl)-6-(5-(methoxymethoxy)-2-methylphenyl)pyrimidine-4-carboxamide (0.28 g, 0.692 mmol) was dissolved in MeOH (5 mL) and treated with 10% Pd/C (0.28 g). The reaction mixture was stirred under an hydrogen atmosphere for 3 h. The reaction mixture was filtered through Celite™ and the filtrate was concentrated. The residue was purified by silica gel chromatography eluting with 2% methanol in dichloromethane to provide 5-amino-2-(4,4-difluorocyclohexyl)-6-(5-(methoxymethoxy)-2-methylphenyl)pyrimidine-4-carboxamide (0.19 g, 68% yield). MS: [M+H]⁺: 407.3.

Step 3. 5-Amino-2-(4,4-difluorocyclohexyl)-6-(5-(methoxymethoxy)-2-methylphenyl)pyrimidine-4-carboxamide (0.19 g, 0.586 mmol) was dissolved in dichloromethane (5 mL) and 4 M HCl solution in dioxane (2 mL) was added at −10° C. The reaction mixture was stirred at 0° C. for 2 h. The reaction mixture was concentrated and the residue was purified by preparative HPLC C18 column eluting with ACN/water/0.1% formic acid to provide 5-amino-2-(4,4-difluorocyclohexyl)-6-(5-hydroxy-2-methylphenyl)pyrimidine-4-carboxamide (0.11 g, 65% yield). MS: [M+H]⁺: 363.3; ¹H NMR (400 MHz, DMSO-d6) δ 9.43 (s, 1H), 8.18 (s, 1H), 7.77 (s, 1H), 7.16 (d, J=8 Hz, 1H), 6.80 (d, J=8 Hz, 1H), 6.66 (s, 1H), 6.22 (bs, 2H), 2.90 (bs, 1H), 2.05-0.03 (m, 4H), 1.99 (s, 3H), 1.88 (bs, 4H).

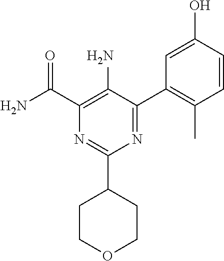 [SMILES: Cc1ccc(O)cc1-c1nc(C2CCOCC2)nc(C(N)=O)c1N]

### Compound 44 (5-amino-6-(5-hydroxy-2-methylphenyl)-2-(tetrahydro-2H-pyran-4-yl)pyrimidine-4-carboxamide)

Step 1. A MW vial was charged with Intermediate B (0.3 g, 0.925 mmol), dioxane-water 9:1 (5 mL), Cs₂CO₃ (0.909 g, 2.78 mmol) and 2-(3,6-dihydro-2H-pyran-4-yl)-4,4,5,5-tetramethyl-1,3,2-dioxaborolane (0.39 g, 1.85 mmol). The reaction mixture was purged with N2 gas for 10 min. Xphos Pd G3 (0.078 g, 0.0929 mmol) was added and the reaction mixture was stirred at 140° C. for 2.5 h. The cooled reaction mixture was diluted with water (20 mL) and extracted with EtOAc (3×10 mL). The combined organic layer was dried over Na₂SO₄, filtered and concentrated. The residue was purified by silica gel chromatography eluting with 35% ethyl acetate in hexane to provide 5-amino-2-(3,6-dihydro-2H-pyran-4-yl)-6-(5-(methoxymethoxy)-2-methylphenyl)pyrimidine-4-carboxamide (0.3 g, 87% yield). MS: [M+H]⁺: 371.3.

Step 2. 5-Amino-2-(3,6-dihydro-2H-pyran-4-yl)-6-(5-(methoxymethoxy)-2-methylphenyl)pyrimidine-4-carboxamide (0.3 g, 0.8077 mmol) was dissolved in MeOH (10 mL) and treated with 10% Pd/C (0.3 g). The reaction mixture was stirred under an hydrogen atmosphere for 16 h. The reaction mixture was filtered through Celite™ and the filtrate was concentrated. The residue was purified by silica gel chromatography eluting with 35% EtOAc in hexanes to provide 5-amino-6-(5-(methoxymethoxy)-2-methylphenyl)-2-(tetrahydro-2H-pyran-4-yl)pyrimidine-4-carboxamide (0.16 g, 53% yield). LCMS: MS: [M+H]⁺: 373.3.

Step 3. 5-Amino-6-(5-(methoxymethoxy)-2-methylphenyl)-2-(tetrahydro-2H-pyran-4-yl)pyrimidine-4-carboxamide (0.16 g, 0.429 mmol) was dissolved in dichloromethane (2 mL) and 4 M HCl solution in dioxane (1.6 mL) was added at −10° C. The reaction mixture was stirred at 0° C. for 3 h. The reaction mixture was concentrated and the residue was purified by preparative HPLC C18 column eluting with ACN/water/0.1% formic acid to provide 5-amino-6-(5-hydroxy-2-methylphenyl)-2-(tetrahydro-2H-pyran-4-yl)pyrimidine-4-carboxamide. (0.015 g, 11% yield). MS: [M+H]⁺: 328.6; ¹H NMR (400 MHz, DMSO-d6) δ 9.44 (bs, 1H), 8.20 (s, 1H), 7.76 (s, 1H), 7.17 (d, J=8.4 Hz, 1H), 6.81 (d, J=2 Hz, 1H), 6.67 (d, J=2 Hz, 1H), 6.22 (bs, 2H), 3.93 (d, J=10.8 Hz, 1H), 3.47-3.42 (m, 3H), 2.99-2.92 (m, 1H), 2.00 (s, 3H), 1.89-1.76 (m, 4H).

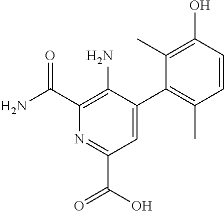 [SMILES: Cc1ccc(O)c(C)c1-c1cc(C(=O)O)nc(C(N)=O)c1N]

### Compound 53 (5-amino-6-carbamoyl-4-(3-hydroxy-2,6-dimethylphenyl)picolinic acid)

To a solution of Intermediate F (12 mg, 38.06 μmol) in DCM (200 μL) was added 1 M BBr₃ solution in DCM (200 μL, 0.2 mmol). The solution was stirred for 135 min then concentrated and co-evaporated with MeOH (2×). The residue was purified by preparative HPLC C18 column eluting with ACN/water/0.1% formic acid to provide 5-amino-6-carbamoyl-4-(3-hydroxy-2,6-dimethyl-phenyl)pyridine-2-carboxylic acid (5 mg, 44% yield). MS: [M+H]⁺: 302.2; ¹H NMR (400 MHz, DMSO-d6) δ 9.36 (br s, 1H), 8.88 (br s, 1H), 7.55 (s, 1H), 6.99 (d, J=8.2 Hz, 1H), 6.81 (d, J=8.2 Hz, 1H), 1.83 (s, 3H), 1.76 (s, 3H).

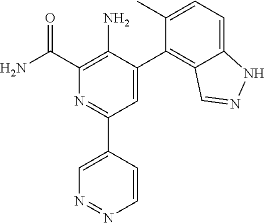 [SMILES: Cc1ccc2[nH]ncc2c1-c1cc(-c2ccnnc2)nc(C(N)=O)c1N]

### Compound 69 (3-amino-4-(5-methyl-1H-indazol-4-yl)-6-(pyridazin-4-yl)picolinamide)

A solution of Intermediate K (100 mg, 347.6 μmol), tributyl(pyridazin-4-yl)stannane (128 mg, 347.6 μmol), copper(I) iodide (8.4 mg, 43.9 μmol), LiCl (30.4 mg, 717.5 μmol) and Pd(dppf)Cl₂·DCM (26.6 mg, 33.8 μmol) in DMF (3 mL) was degassed in vacuo, back-filled with N₂, then stirred at 130° C. for 2 h.

The solution was filtered and then purified by preparative HPLC C18 column eluting with ACN/water/0.1% formic acid to provide 3-amino-4-(1H-indazol-4-yl)-6-pyridazin-4-yl-pyridine-2-carboxamide (12 mg, 10% yield). MS: [M+H]⁺: 332.3; ¹H NMR (400 MHz, DMSO-d6) δ 13.26 (s, 1H), 10.07 (dd, J=2.5, 1.2 Hz, 1H), 9.15 (dd, J=5.5, 1.2 Hz, 1H), 8.52 (d, J=2.6 Hz, 1H), 8.37 (dd, J=5.5, 2.5 Hz, 1H), 8.13 (s, 1H), 7.86 (s, 1H), 7.69-7.54 (m, 2H), 7.57-7.38 (m, 1H), 7.20 (dd, J=7.0, 0.8 Hz, 1H), 6.98 (s, 1H).

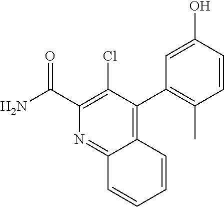 [SMILES: Cc1ccc(O)cc1-c1c(Cl)c(C(N)=O)nc2ccccc12]

### Compound 73 (3-chloro-4-(5-hydroxy-2-methylphenyl)quinoline-2-carboxamide)

Step 1. To a suspension of 4-oxo-1H-quinoline-2-carboxylic acid (5.00 g, 26.43 mmol) in MeOH (10 mL) was added concentrated sulfuric acid (141 μL, 2.64 mmol). The reaction mixture was stirred under reflux for 72 h. The pH-adjusted was to 4 and MeOH was removed under reduced pressure. The residue was purified using flash silica gel chromatography eluting with MeOH/DCM 0-20% to provide methyl 4-oxo-1H-quinoline-2-carboxylate (350 mg, 7% yield). MS: [M+H]⁺: 204.2.

Step 2. A suspension of methyl 4-oxo-1H-quinoline-2-carboxylate (350 mg, 1.72 mmol) and NCS (253 mg, 1.89 mmol) dissolved in ACN (10 mL) and acetic acid (0.5 mL) was stirred at 90° C. for 17 h. Solids were filtered and the filtrate was concentrated to provide methyl 3-chloro-4-hydroxy-quinoline-2-carboxylate (376 mg, 92% yield). MS: [M+H]⁺: 238.1.

Step 3. To a solution of methyl 3-chloro-4-hydroxy-quinoline-2-carboxylate (376 mg, 1.58 mmol) in pyridine (10 mL) at 0° C. was added trifluoromethylsulfonyl trifluoromethanesulfonate (1 M in DCM, 2.1 mL, 2.1 mmol) and the reaction was stirred from 0° C. to rt for 17 h. Solvents were removed under reduced pressure and the residue was purified using flash silica gel chromatography eluting with EtOAc/hexanes 5-95% to provide methyl 3-chloro-4-(trifluoromethylsulfonyloxy)quinoline-2-carboxylate (182 mg, 31% yield). MS: [M+H]⁺: 370.0.

Step 4. [5-(methoxymethoxy)-2-methyl-phenyl]boronic acid (106.14 mg, 541.52 μmol), potassium carbonate (238 mg, 1.72 mmol), Pd(PPh₃)₄ (57 mg, 49.23 μmol) and methyl 3-chloro-4-(trifluoromethylsulfonyloxy)quinoline-2-carboxylate (182 mg, 492.3 μmol) in toluene (3 mL) and water (0.8 mL) were degassed under N₂ and then stirred under reflux for 3 h. The solvents were removed under reduced pressure and the residue was purified using flash silica gel chromatography eluting with EtOAc/hexanes 5-95% to provide methyl 3-chloro-4-[5-(methoxymethoxy)-2-methyl-phenyl]quinoline-2-carboxylate (91 mg, 50% yield). MS: [M+H]⁺: 372.1.

Step 5. A MW vial was charged with methyl 3-chloro-4-[5-(methoxymethoxy)-2-methyl-phenyl]quinoline-2-carboxylate (90 mg, 242.06 μmol) and MeOH (3.5 mL). 7 N Ammonia solution in MeOH (3.5 mL, 24.5 mmol) was added and the vessel was sealed and stirred at 110° C. for 3 h. The solvents were removed under reduced pressure. The solid was suspended in DCM (1 mL), then treated with 4 M HCl solution in dioxane (380 μL, 1.52 mmol) and the mixture was stirred at rt for 2 h. The volatiles were removed in vacuo and the residue was purified by preparative HPLC C18 column eluting with ACN/water/0.1% formic acid to provide 3-chloro-4-(5-hydroxy-2-methyl-phenyl)quinoline-2-carboxamide (3 mg, 4% yield). MS: [M+H]⁺: 313.1. ¹H NMR (400 MHz, Chloroform-d) δ 8.11 (ddd, J=8.5, 1.3, 0.7 Hz, 1H), 7.73 (ddd, J=8.4, 6.9, 1.4 Hz, 1H), 7.56-7.43 (m, 2H), 7.32 (ddd, J=8.5, 1.4, 0.7 Hz, 1H), 6.90 (dd, J=8.3, 2.7 Hz, 1H), 6.59 (d, J=2.7 Hz, 1H), 5.68 (s, 1H), 4.95 (s, 1H), 1.85 (s, 3H).

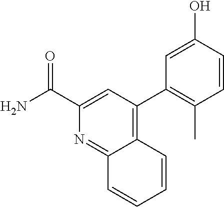 [SMILES: Cc1ccc(O)cc1-c1cc(C(N)=O)nc2ccccc12]

### Compound 74 (4-(5-hydroxy-2-methylphenyl)quinoline-2-carboxamide)

To a solution of compound 73 (10.0 mg, 31.97 μmol) in DMF (2 mL) were added cesium carbonate (21.0 mg, 63.95 μmol), (2,4-dimethoxyphenyl)methanamine (6.6 mg, 39.65 μmol) and Pd₂(dba)₃-tri-tert-butyl phosphonium tetrafluoroborate mixture (mole ratio: 1/1.2) (3.9 mg, 3.2 μmol) in a sealed tube. The mixture was degassed in vacuo, back-filled with N2 and stirred at 120° C. for 17 h. The volatiles were removed in vacuo and the residue was purified by preparative HPLC C18 column eluting with ACN/water/0.1% formic acid to provide 4-(5-hydroxy-2-methyl-phenyl)quinoline-2-carboxamide (3.1 mg, 35% yield). MS: [M+H]⁺: 279.1. ¹H NMR (400 MHz, Chloroform-d) δ 8.22-8.04 (m, 3H), 7.81-7.69 (m, 1H), 7.66-7.41 (m, 2H), 7.21-7.14 (m, 1H), 6.87 (dd, J=8.3, 2.7 Hz, 1H), 6.70 (d, J=2.7 Hz, 1H), 5.68 (s, 1H), 1.89 (s, 3H).

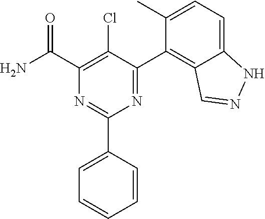 [SMILES: Cc1ccc2[nH]ncc2c1-c1nc(-c2ccccc2)nc(C(N)=O)c1Cl]

### Compound 75 (5-chloro-6-(5-methyl-1H-indazol-4-yl)-2-phenylpyrimidine-4-carboxamide)

Step 1. A mixture of methyl 2,5,6-trichloropyrimidine-4-carboxylate (1.00 g, 4.14 mmol), (5-methyl-1H-indazol-4-yl)boronic acid (771 mg, 4.38 mmol) and sodium carbonate (880 mg, 8.30 mmol) in dioxane (10 mL) and water (1.5 mL) was bubbled through with N₂, then Pd(PPh₃)₄ (240 mg, 207.7 μmol) was added. The mixture was stirred under N₂ at 80° C. for 2.5 h. The cooled reaction mixture was filtered and the filtrate was diluted with water and extracted with EtOAc (3×). The combined organic layers were washed with brine, dried over Na₂SO₄, filtered, and adsorbed on silica. The residue was purified by silica gel chromatography eluting with a gradient of EtOAc (0 to 100%) in heptane to afford methyl 2,5-dichloro-6-(5-methyl-1H-indazol-4-yl)pyrimidine-4-carboxylate (268 mg, 19% yield). MS: [M+H]⁺: 337.0.

Step 2. A solution of methyl 2,5-dichloro-6-(5-methyl-1H-indazol-4-yl)pyrimidine-4-carboxylate (103 mg, 305.5 μmol), 4,4,5,5-tetramethyl-2-phenyl-1,3,2-dioxaborolane (68 mg, 333.2 μmol) and Pd(dppf)Cl₂·DCM (28 mg, 34.3 μmol) in dioxane (1 mL) and 2 M aqueous solution of potassium carbonate (460 μL, 0.92 mmol) was degassed by bubbling N2. The reaction mixture was then stirred at 100° C. for 4 h, cooled to rt, acidified with 1 N aqueous HCl solution and extracted with CHCl₃/iPrOH 4:1 (3×). Combined organic extracts were concentrated and dried in vacuo. The residue was dissolved in DMF (1 mL) then treated with ammonium chloride (81 mg, 1.52 mmol) and HATU (148 mg, 389.24 μmol). DIPEA (320 μL, 1.84 mmol) was added and the solution was stirred at rt for 3 h. The crude reaction mixture was filtered, and the filtrate was purified by preparative HPLC C18 column eluting with ACN/water/0.1% formic acid to provide 5-chloro-6-(5-methyl-1H-indazol-4-yl)-2-phenyl-pyrimidine-4-carboxamide (23 mg, 21% yield). MS: [M+H]⁺: 364.1. ¹H NMR (400 MHz, DMSO-d6) δ 13.25 (s, 1H), 8.42 (s, 1H), 8.40-8.38 (m, 1H), 8.38-8.33 (m, 1H), 8.13 (s, 1H), 7.70 (s, 1H), 7.64 (d, J=8.6 Hz, 1H), 7.60-7.48 (m, 3H), 7.39 (d, J=8.6 Hz, 1H), 2.27 (s, 3H).

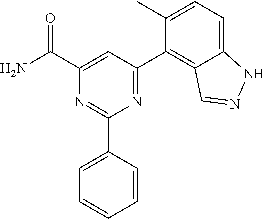 [SMILES: Cc1ccc2[nH]ncc2c1-c1cc(C(N)=O)nc(-c2ccccc2)n1]

### Compound 76 (6-(5-methyl-1H-indazol-4-yl)-2-phenylpyrimidine-4-carboxamide)

Step 1. A mixture of methyl 2,6-dichloropyrimidine-4-carboxylate (1.05 g, 5.07 mmol), (5-methyl-1H-indazol-4-yl)boronic acid (909 mg, 5.17 mmol) and sodium carbonate (1.08 g, 10.17 mmol) in dioxane (10 mL) and water (1.5 mL) was bubbled through with N₂. Pd(PPh₃)₄ (300 mg, 259.6 μmol) was added and the mixture was stirred under N₂ at 80° C. overnight. The reaction mixture was cooled to rt, diluted with 1 N aqueous HCl solution and water. The solids were collected by filtration on Buchner and washed with water then air dried and purified by reverse phase flash chromatography eluting with a gradient of acetonitrile (10 to 100%) in water both containing 0.1% formic acid to provide 2-chloro-6-(5-methyl-1H-indazol-4-yl)pyrimidine-4-carboxylic acid.

Step 2. A solution of 2-chloro-6-(5-methyl-1H-indazol-4-yl)pyrimidine-4-carboxylic acid (208 mg, 720.5 μmol), 4,4,5,5-tetramethyl-2-phenyl-1,3,2-dioxaborolane (177 mg, 867.3 μmol) and Pd(dppf)Cl₂·DCM (65 mg, 79.6 μmol) in dioxane (2 mL) and 2 M aqueous solution of potassium carbonate (1.08 mL, 2.16 mmol) was degassed by bubbling N₂. The reaction mixture was stirred at 100° C. for 2 h 20 min then cooled to rt, acidified with 1 N aqueous HCl solution and extracted with CHCl₃/iPrOH 4:1. The combined organic extracts were dried over MgSO₄, filtered, concentrated and dried in vacuo, affording crude 6-(5-methyl-1H-indazol-4-yl)-2-phenyl-pyrimidine-4-carboxylic acid (335 mg). A solution containing 238 mg of this crude solid in DMF (2 mL) was treated with ammonium chloride (201 mg, 3.76 mmol) and HATU (350 mg, 920.5 μmol) followed by the addition of DIPEA (760 μL 4.35 mmol). The mixture was stirred at rt for 1 h. The crude reaction mixture was filtered, and the filtrate was purified by preparative HPLC (Phenomenex Gemini®) eluting with a gradient of acetonitrile in water both containing 0.1% formic acid to provide 6-(5-methyl-1H-indazol-4-yl)-2-phenyl-pyrimidine-4-carboxamide (15 mg, 6% yield). MS: [M+H]⁺: 330.1. ¹H NMR (400 MHz, DMSO-d6) δ 13.25 (s, 1H), 8.73 (s, 1H), 8.69 (dd, J=7.5, 2.4 Hz, 2H), 8.10 (s, 1H), 8.05 (d, J=1.2 Hz, 1H), 7.96 (s, 1H), 7.64 (d, J=8.6 Hz, 1H), 7.61-7.51 (m, 3H), 7.40 (d, J=8.5 Hz, 1H), 2.50 (s, 3H).

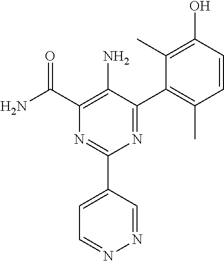 [SMILES: Cc1ccc(O)c(C)c1-c1nc(-c2ccnnc2)nc(C(N)=O)c1N]

### Compound 77 (6-(3-hydroxy-2,6-dimethylphenyl)-2-(pyridazin-4-yl)pyrimidine-4-carboxamide)

Step 1. To a solution of Intermediate H (120 mg, 392.5 μmol) in DMF (1 mL) were added lithium chloride (41 mg, 967 μmol), tributyl(pyridazin-4-yl)stannane (290 mg, 786 μmol), copper(I) iodide (18 mg, 95 μmol), and Pd(dppf)Cl₂ (29 mg, 39.6 μmol). The mixture was stirred at 120° C. for 2 h. The crude mixture was purified using a prep-HPLC C18 column eluting with ACN/water/0.1% formic acid to provide 3-amino-2-(3-methoxy-2,6-dimethyl-phenyl)-6-pyridazin-4-yl-pyridine-4-carboxamide (75 mg, 55% yield).

Step 2. 1 M Boron tribromide solution in DCM (1.2 mL, 1.2 mmol) was added to a solution of 3-amino-2-(3-methoxy-2,6-dimethyl-phenyl)-6-pyridazin-4-yl-pyridine-4-carboxamide (130 mg, 372.1 μmol) in DCM (1.5 mL). After 30 min the volatiles were removed, and the residue was taken in MeOH. The MeOH was evaporated to dryness. The residue was taken in MeOH and 1 mL of Et₃N was added. The mixture was evaporated to dryness. The residue was dissolved in 10% MeOH/DCM and adsorbed on silica gel. The residue was purified by silica gel chromatography eluting with a gradient of MeOH (0 to 10%) in DCM to provide 3-amino-2-(3-hydroxy-2,6-dimethyl-phenyl)-6-pyridazin-4-yl-pyridine-4-carboxamide (76 mg, 55% yield). MS: [M+H]+: 336.3. 1H NMR (400 MHz, DMSO-d6) δ 9.86 (dd, J=2.5, 1.1 Hz, 1H), 9.32 (dd, J=5.9, 1.1 Hz, 1H), 8.53 (s, 1H), 8.41 (dd, J=5.9, 2.4 Hz, 1H), 8.34 (s, 1H), 7.84-7.71 (m, 1H), 6.95 (t, J=7.4 Hz, 1H), 6.80 (d, J=8.2 Hz, 1H), 1.83 (s, 3H), 1.76 (s, 3H).

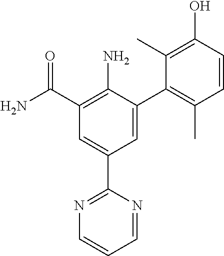 [SMILES: Cc1ccc(O)c(C)c1-c1cc(-c2ncccn2)cc(C(N)=O)c1N]

### Compound 78 (3′-hydroxy-2′,6′-dimethyl-5-(pyrimidin-2-yl)-[1,1′-biphenyl]-3-carboxamide)

Step 1. In pressure vessel, a mixture of Intermediate 1 (0.130 g, 0.43 mmol), in dioxane (5 mL), water (1 mL), Cs₂CO₃ (0.283 g, 0.86 mmol) and 2-bromopyrimidine (0.082 g, 0.51 mmol) was purged with N₂ gas for 10 min, followed by addition of PdCl₂(dppf)·DCM (0.035 g, 0.043 mmol). The reaction mixture was stirred at 100° C. for 2 h. The reaction mixture was quenched with water (10 mL) and extracted with EtOAc (3×10 mL). The combined organic layer was dried over Na₂SO₄, filtered and concentrated. The residue was purified by silica gel chromatography eluting with 35% EtOAc in hexanes to provide 2-amino-3′-(methoxymethoxy)-2′,6′-dimethyl-5-(pyrimidin-2-yl)-[1,1′-biphenyl]-3-carboxamide (0.08 g, 69%). MS: [M+H]⁺: 379.4.

Step 2. 2-Amino-3′-(methoxymethoxy)-2′,6′-dimethyl-5-(pyrimidin-2-yl)-[1,1′-biphenyl]-3-carboxamide (0.1 g, 0.26 mmol) was dissolved in DCM (5 mL) followed by addition of 1-propanethiol (0.08 g, 1.05 mmol) and ZnBr₂ (0.120 g 0.52 mmol). The reaction mixture was stirred at rt for 3 h. More 1-propanethiol (0.08 g, 1.05 mmol) and ZnBr₂ (0.07 g 0.31 mmol) were added and the reaction was stirred for 12 h at rt. The reaction mixture was poured into cold water (10 mL) and extracted with EtOAc (3×10 mL). The combined organic layers was dried over Na₂SO₄ and concentrated. The residue was purified by silica gel chromatography eluting with a gradient of MeOH (0 to 5%) in DCM to provide 2-amino-3′-hydroxy-2′,6′-dimethyl-5-(pyrimidin-2-yl)-[1,1′-biphenyl]-3-carboxamide (0.012 g, 14%). MS: [M+H]⁺: 335.3 ¹H NMR (400 MHz, DMSO-d6) δ 9.19 (d, J=4.8 Hz, 1H), 8.77-8.76 (m, 2H), 8.61 (d, J=4.0 Hz, 1H), 8.09 (bs, 1H), 7.89 (d, J=4.0 Hz, 2H), 7.27 (d, J=4.8 Hz, 2H), 6.96 (d, J=6.8 Hz, 1H), (t, J=7.6 Hz, 1H), 6.14 (bs, 2H), 1.87 (s, 3H), 1.80 (s, 3H).

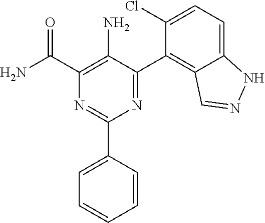 [SMILES: NC(=O)c1nc(-c2ccccc2)nc(-c2c(Cl)ccc3[nH]ncc23)c1N]

### Compound 115 (5-amino-6-(5-chloro-1H-indazol-4-yl)-2-phenylpyrimidine-4-carboxamide)

Step 1. A MW vial was charged with Intermediate L (175 mg, 543.21 μmol), 5-chloro-1-tetrahydropyran-2-yl-4-(4,4,5,5-tetramethyl-1,3,2-dioxaborolan-2-yl)indazole (213 mg, 587.3 μmol), dioxane (3 mL) and 2 M aqueous K₃PO₄ solution (543 μL, 1.09 mmol). The solution was degassed then Pd₂(dba)₃ (53 mg, 57.9 μmol) and tri-tert-butylphosphonium tetrafluoroborate (53 mg, 182.7 μmol) were added. The solution was degassed again, capped and transferred to a preheated heat block (90° C.). After stirring for 1 h, the cooled solution was diluted with water and extracted with DCM (3×). The combined extracts was concentrated and the residue was purified by silica gel chromatography eluting with a gradient of EtOAc (0 to 100%) in heptane to provide ethyl 5-amino-6-(5-chloro-1-tetrahydropyran-2-yl-indazol-4-yl)-2-phenyl-pyrimidine-4-carboxylate (197 mg, 76% yield). MS: [M+H]⁺: 478.4.

Step 2. Ethyl 5-amino-6-(5-chloro-1-tetrahydropyran-2-yl-indazol-4-yl)-2-phenyl-pyrimidine-4-carboxylate (197 mg, 412.2 μmol) and 7 N ammonia solution in methanol (10 mL, 70 mmol) were charged in a pressure vessel. The vessel was sealed and transferred to a preheated (100° C.) pellet bath. After stirring overnight the solution was cooled to rt and concentrated then dried in vacuo. MeOH (5 mL) was added to the resulting solid followed by 6 N aqueous HCl solution (1 mL, 6 mmol). The solution was stirred at 50° C. for 3 h. The mixture was concentrated to dryness and the residue was coevaporated with MeOH and Et₃N then purified by preparative HPLC C18 column eluting with ACN/water/0.1% formic acid to provide 5-amino-6-(5-chloro-1H-indazol-4-yl)-2-phenyl-pyrimidine-4-carboxamide (102 mg, 68% yield). MS: [M+H]⁺: 365.4; ¹H NMR (400 MHz, DMSO-d6) δ 13.45 (br s, 1H), 8.61 (br s, 1H), 8.45-8.31 (m, 2H), 7.88 (br s, 1H), 7.79 (br s, 1H), 7.74 (dd, J=8.9, 1.0 Hz, 1H), 7.55 (d, J=8.9 Hz, 1H), 7.48-7.32 (m, 3H), 6.66 (br s, 2H).

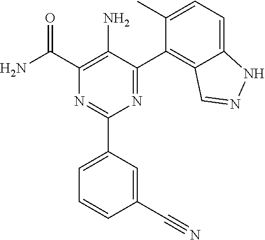 [SMILES: Cc1ccc2[nH]ncc2c1-c1nc(-c2cccc(C#N)c2)nc(C(N)=O)c1N]

### Compound 131 (5-amino-2-(3-cyanophenyl)-6-(5-methyl-1H-indazol-4-yl)pyrimidine-4-carboxamide)

To a solution of Intermediate J (50 mg, 165.2 μmol), (3-cyanophenyl)boronic acid (48 mg, 326.7 μmol), and Pd(dppf)Cl₂·DCM (15 mg, 18.4 μmol) in dioxane (1.5 mL) was added 2 M aqueous K₂CO₃ solution (330 μL, 660 μmol). The reaction mixture was stirred at 100° C. for 5 h. The crude reaction mixture was cooled to rt, filtered, and the filtrate was purified by preparative HPLC C18 column eluting with ACN/water/0.1% formic acid to provide 5-amino-2-(3-cyanophenyl)-6-(5-methyl-1H-indazol-4-yl)pyrimidine-4-carboxamide (4.2 mg, 7% yield). MS: [M+H]⁺: 370.3; ¹H NMR (400 MHz, DMSO-d6) δ 13.13 (s, 1H), 8.92 (t, J=1.7 Hz, 1H), 8.81 (d, J=2.1 Hz, 1H), 8.58 (ddd, J=8.1, 1.8, 1.2 Hz, 1H), 7.85 (d, J=2.3 Hz, 1H), 7.80 (dt, J=7.7, 1.4 Hz, 1H), 7.64 (s, 1H), 7.61-7.54 (m, 2H), 7.34 (d, J=8.6 Hz, 1H), 2.21 (s, 3H).

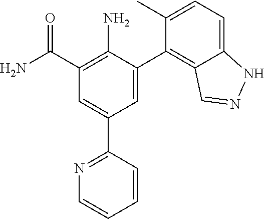 [SMILES: Cc1ccc2[nH]ncc2c1-c1cc(-c2ccccn2)cc(C(N)=O)c1N]

Compound 137 (2-amino-3-(5-methyl-1H-indazol-4-yl)-5-(pyridin-2-yl)benzamide)

A solution of tributyl(2-pyridyl)stannane (67 mg, 182.3 μmol), 2-amino-5-bromo-3-(5-methyl-1H-indazol-4-yl)benzamide (57 mg, 165.7 μmol), copper(I) iodide (4.0 mg, 20.9 μmol), LiCl (14.5 mg, 342.1 μmol) and Pd(dppf)Cl₂·DCM (12.7 mg, 16.1 μmol) in DMF (3 mL) was degassed in vacuo and back-filled with N₂. It was then stirred at 120° C. for 18 h. The mixture was filtered and the filtrate was purified by preparative HPLC C18 column eluting with ACN/water/0.1% formic acid to provide 2-amino-3-(5-methyl-1H-indazol-4-yl)-5-(2-pyridyl)benzamide (3 mg, 5% yield). MS: [M+H]⁺: 344.3; ¹H NMR (400 MHz, DMSO-d6) δ 13.02 (s, 1H), 8.48 (d, J=4.9 Hz, 1H), 8.32 (d, J=2.2 Hz, 1H), 8.10 (s, 1H), 7.94 (d, J=8.2 Hz, 1H), 7.86 (d, J=2.1 Hz, 1H), 7.76 (t, J=7.7 Hz, 1H), 7.46 (d, J=9.2 Hz, 2H), 7.31 (d, J=8.6 Hz, 2H), 7.22-7.08 (m, 1H), 6.17 (s, 2H), 2.15 (s, 3H).

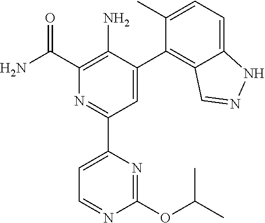 [SMILES: Cc1ccc2[nH]ncc2c1-c1cc(-c2ccnc(OC(C)C)n2)nc(C(N)=O)c1N]

### Compound 160 (3-amino-6-(2-isopropoxypyrimidin-4-yl)-4-(5-methyl-1H-indazol-4-yl)picolinamide)

To a solution of propan-2-ol (17 mg, 0.275 mmol) in DMF (1 mL) was added NaH (60% in oil) (8 mg, 0.189 mmol) at 0° C. The mixture was stirred at 0° C. for 30 min. Intermediate N (0.04 g, 0.0945 mmol) was then added and the reaction mixture was stirred at rt for 2 h. The reaction mixture was quenched with water (10 mL) and extracted with EtOAc (3×10 mL). The combined organic layer was dried over Na₂SO₄, filtered and concentrated. The residue was purified by preparative HPLC C18 column eluting with ACN/water/0.1% formic acid to provide 3-amino-6-(2-isopropoxypyrimidin-4-yl)-4-(5-methyl-1H-indazol-4-yl)picolinamide (10 mg, 26% yield). MS: [M+H]⁺: 404.6; ¹H NMR (400 MHz, DMSO-d6) δ 13.09 (bs, 1H), 8.61 (d, J=5.2 Hz, 1H), 8.54 (s, 1H), 8.33 (d, J=5.2 Hz, 1H), 8.11 (s, 1H), 7.67 (s, 1H), 7.58-7.56 (m, 2H), 7.37 (d, J=8.4 Hz, 1H), 5.21-5.15 (m, 1H), 2.19 (s, 3H), 1.94 (dd, J=6.0, 2.0 Hz, 6H).

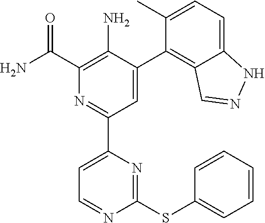 [SMILES: Cc1ccc2[nH]ncc2c1-c1cc(-c2ccnc(Sc3ccccc3)n2)nc(C(N)=O)c1N]

### Compound 177 (3-amino-4-(5-methyl-1H-indazol-4-yl)-6-(2-(phenylthio)pyrimidin-4-yl)picolinamide)

In a MW vial, Intermediate N (0.05 g, 0.118 mmol) was dissolved in DMF (1 mL) and treated with K₂CO₃ (0.049 g, 0.3542 mmol) and thiophenol (0.026 g, 0.236 mmol). The resulting mixture was stirred at 100° C. for 2 h in microwave. The reaction mixture was filtered through Celite™ and the filtrate was concentrated. The residue was purified by preparative HPLC C18 column eluting with ACN/water/0.1% formic acid to provide 3-amino-4-(5-methyl-1H-indazol-4-yl)-6-(2-(phenylthio)pyrimidin-4-yl)picolinamide (0.015 g, 28% yield). MS: [M+H]⁺: 454.4; ¹H NMR (400 MHz, DMSO-d6) δ 13.18 (s, 1H), 8.63 (d, J=5.2 Hz, 1H), 8.54 (s, 1H), 8.43 (d, J=5.2 Hz, 1H), 7.77 (s, 1H), 7.65 (s, 1H), 7.60-7.56 (m, 3H), 7.38 (d, J=8.4 Hz, 1H), 7.21 (t, J=7.2 Hz, 7.6 Hz, 2H), 7.11 (t, J=7.2 Hz, 1H), 2.14 (s, 3H).

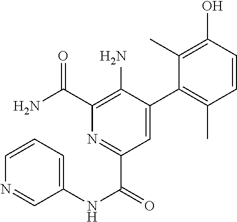 [SMILES: Cc1ccc(O)c(C)c1-c1cc(C(=O)Nc2cccnc2)nc(C(N)=O)c1N]

### Compound 188 (5-amino-4-(3-hydroxy-2,6-dimethylphenyl)-N2-(pyridin-3-yl)pyridine-2,6-dicarboxamide)

To a vial containing 5-amino-6-carbamoyl-4-(3-hydroxy-2,6-dimethyl-phenyl)pyridine-2-carboxylic acid (30 mg, 99.6 μmol), pyridin-3-amine (10 mg, 106.3 μmol) and HATU (45 mg, 118.4 μmol) was added DMF (300 μL) and DIPEA (70 μL, 401.9 μmol,). The mixture was stirred at rt for 70 min, then the crude reaction mixture was filtered, and the filtrate was purified by preparative HPLC C18 column eluting with ACN/water 10 mM ammonium bicarbonate to provide 5-amino-4-(3-hydroxy-2,6-dimethylphenyl)-N₂-(pyridin-3-yl)pyridine-2,6-dicarboxamide (4 mg, 11% yield). MS: [M+H]⁺: 378.2; ¹H NMR (400 MHz, DMSO-d6) δ 10.64 (s, 1H), 9.41 (br s, 1H), 9.08 (d, J=2.5 Hz, 1H), 9.01-8.88 (m, 1H), 8.39 (br s, 1H), 8.33 (dd, J=4.7, 1.5 Hz, 1H), 8.19 (ddd, J=8.3, 2.6, 1.5 Hz, 1H), 7.73 (d, J=2.5 Hz, 1H), 7.66 (s, 1H), 7.42 (ddd, J=8.3, 4.7, 0.8 Hz, 1H), 7.01 (d, J=8.2 Hz, 1H), 6.82 (d, J=8.2 Hz, 1H), 1.85 (s, 3H), 1.78 (s, 3H).

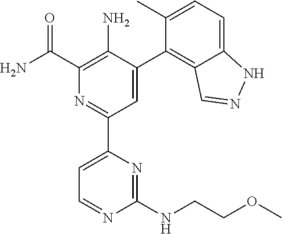 [SMILES: COCCNc1nccc(-c2cc(-c3c(C)ccc4[nH]ncc34)c(N)c(C(N)=O)n2)n1]

### Compound 214 (3-amino-6-(2-((2-methoxyethyl)amino)pyrimidin-4-yl)-4-(5-methyl-1H-indazol-4-yl)picolinamide)

A microwave vessel was charged with Intermediate N (0.1 g, 0.2364 mmol) and 2-methoxyethan-1-amine (1.5 mL). The vessel was sealed and the mixture was heated at 100° C. for 1 h in MW. The reaction mixture was concentrated and the residue was purified by trituration with hexanes and diethyl-ether to provide 3-amino-6-(2-((2-methoxyethyl)amino)pyrimidin-4-yl)-4-(5-methyl-1H-indazol-4-yl)picolinamide (40 mg, 40% yield). MS: [M+H]⁺: 419.4; ¹H NMR (400 MHz, DMSO-d6) δ 13.15 (s, 1H), 8.42 (bs, 1H), 8.35 (d, J=4.8 Hz, 1H), 8.15 (s, 1H), 7.82 (d, J=5.2 Hz, 1H), 7.62-7.56 (m, 3H), 7.37 (d, J=8.4 Hz, 1H), 6.97 (bs, 1H), 3.40-3.33 (m, 4H), 3.17 (bs, 3H), 2.19 (s, 3H).

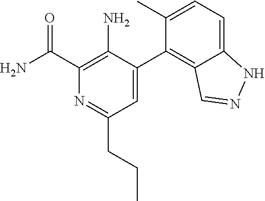 [SMILES: CCCc1cc(-c2c(C)ccc3[nH]ncc23)c(N)c(C(N)=O)n1]

Compound 216 (3-amino-6-butyl-4-(5-methyl-1H-indazol-4-yl)picolinamide) A MW vial was charged with Intermediate K (53 mg, 175.7 μmol), copper(I) iodide (8 mg, 42.1 μmol), LiCl (38 mg, 896.4 μmol) and Pd(dppf)Cl₂·DCM (15 mg, 18.4 μmol). DMF (1 mL) was added and vial was degassed. Tributyl(thiazol-4-yl)stannane (80 μL, 213.8 μmol) was added and the vial was degassed again, capped and transferred to a preheated (130° C.) heat block. After 4.5 h the reaction mixture was cooled to rt, filtered and purified by preparative HPLC C18 column eluting with ACN/water/0.1% formic acid to provide 3-amino-6-butyl-4-(5-methyl-1H-indazol-4-yl)picolinamide (6.5 mg, 20% yield). MS: [M+H]⁺: 324.2; ¹H NMR (400 MHz, DMSO-d6) δ 13.26-12.95 (m, 1H), 8.03 (d, J=3.8 Hz, 1H), 7.52 (dd, J=8.5, 1.0 Hz, 1H), 7.49 (t, J=1.2 Hz, 1H), 7.44 (d, J=3.7 Hz, 1H), 7.33 (d, J=8.6 Hz, 1H), 7.05 (s, 1H), 6.10 (s, 2H), 2.74-2.59 (m, 2H), 2.16 (s, 3H), 1.74-1.60 (m, 2H), 1.31 (dh, J=14.6, 7.4 Hz, 2H), 0.90 (t, J=7.4 Hz, 3H).

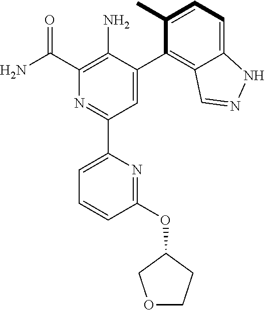

### Compound 227 ((R)-5-amino-4-(5-methyl-1H-indazol-4-yl)-6′-(((R)-tetrahydrofuran-3-yl)oxy)-[2,2′-bipyridine]-6-carboxamide)

To a solution of (3R)-tetrahydrofuran-3-ol (55 μL, 686.7 μmol) and Intermediate O (50 mg, 138.0 mmol) in DMF (1 mL) was added NaH (60% dispersion in mineral oil) (25 mg, 576.70 μmol). The reaction mixture was stirred at 80° C. for 1 h. The reaction mixture was cooled to rt, poured into water and extracted with EtOAc (3×). The combined organic layers was washed with brine, dried over Na₂SO₄, filtered and concentrated. The residue was purified by silica gel chromatography eluting with a gradient of EtOAc (0 to 100%) in heptane to afford a mixture of atropisomers which were separated by chiral SFC to provide (R)-5-amino-4-(5-methyl-1H-indazol-4-yl)-6′-(((R)-tetrahydrofuran-3-yl)oxy)-[2,2′-bipyridine]-6-carboxamide (17 mg, 28% yield) as one of the two atropisomers. MS: [M+H]⁺: 431.3; ¹H NMR (400 MHz, DMSO-d6) δ 13.19-13.01 (m, 1H), 8.37 (d, J=2.9 Hz, 1H), 8.21 (dd, J=7.6, 0.8 Hz, 1H), 8.06 (s, 1H), 7.80-7.70 (m, 1H), 7.62-7.47 (m, 3H), 7.35 (d, J=8.5 Hz, 1H), 6.71 (m, 3H), 5.42 (ddt, J=6.9, 4.7, 2.2 Hz, 1H), 3.83-3.56 (m, 4H), 2.18 (s, 3H), 2.07 (m, 1H), 1.97-1.85 (m, 1H).

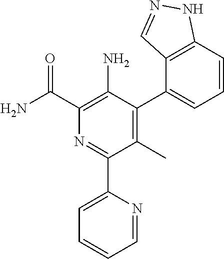 [SMILES: Cc1c(-c2ccccn2)nc(C(N)=O)c(N)c1-c1cccc2[nH]ncc12]

### Compound 246 (5-amino-4-(1H-indazol-4-yl)-3-methyl-[2,2′-bipyridine]-6-carboxamide)

Step 1. To a solution of 3-amino-5-methylpicolinic acid hydrochloride (300 mg, 1.59 mmol) in DMF (15.9 mL) at rt were added HATU (665 mg, 1.749 mmol) and DIPEA (0.830 mL, 4.765 mmol) followed by a dropwise addition of ammonium hydroxide (28-30%, 0.885 mL, 6.363 mmol). The mixture was allowed to stir at rt over weekend. Water (40 mL) and EtOAc (40 mL) were added. The layers were partitioned and the aqueous layer was extracted with EtOAc (30 mL). The combined organic layers was washed with water (2×30 mL) and brine, dried over MgSO₄, filtered and concentrated. The residue was purified by silica gel chromatography eluting with a gradient of EtOAc (30 to 90%) in heptane to provide 3-amino-5-methylpicolinamide (140 mg, 58% yield). MS: [M+H]⁺: 152.1; ¹H NMR (400 MHz, DMSO-d6) δ 7.81 (s; 1H); 7.59 (s; 1H); 7.19 (s; 1H); 6.90 (s; 1H); 6.74 (s; 2H); 2.18 (s; 3H).

Step 2. To a suspension of 3-amino-5-methylpicolinamide (70 mg, 0.463 mmol) in water (3.3 mL) at rt was added sulfuric acid (0.050 mL, 0.938 mmol). A solution of bromine (0.05 mL, 0.976 mmol) in acetic acid (0.32 mL, 5.59 mmol) was added dropwise. The mixture was stirred at rt for 18 h. The reaction mixture was diluted with water (25 mL) and neutralized by addition of solid NaHCO₃ (very exothermic quench). The mixture was extracted with DCM (3×20 mL). The combined organic layers were washed with aqueous saturated NaHCO₃, water and brine, dried over MgSO₄, filtered and concentrated. The residue was triturated in 50% EtOAc/hexanes to afford 3-amino-4,6-dibromo-5-methylpicolinamide (62 mg, 43% yield). MS: [M+H]⁺: 309.9; ¹H NMR (400 MHz, DMSO-d6) δ 7.82 (s; 1H); 7.60 (s; 1H); 7.17 (br s; 2H); 2.47 (s; 3H).

Step 3. To a degassed solution of 3-amino-4,6-dibromo-5-methylpicolinamide (102 mg, 0.33 mmol) in THF (2 mL) were added 2-(tributylstannyl)pyridine (85%, 143 mg, 0.33 mmol), LiCl (29 mg, 0.668 mmol), CuI (13 mg, 0.068 mmol) and Pd(PPh₃)₄ (38 mg, 0.033 mmol). The mixture was degassed and back-filled with argon (3 cycles). The mixture was heated to reflux for 18 h. Upon cooling to rt, the volatiles were evaporated. DCM (15 mL) was added, followed 28-30% NH₄OH (0.640 mL) and EDTA (120 mg). The mixture was heated to 40° C. for 1 h and diluted with water and DCM. The layers were separated and the aqueous layer was extracted with DCM. The combined organic layers was washed with brine, dried over MgSO₄, filtered and concentrated. The residue was purified by silica gel chromatography eluting with a gradient of acetonitrile (10 to 80%) in DCM to provide 5-amino-4-bromo-3-methyl-[2,2′-bipyridine]-6-carboxamide (78 mg, 77% yield). MS: [M+H]⁺: 306.9/308.9; ¹H NMR (400 MHz, DMSO-d6) δ 8.59 (d; J=4.78 Hz; 1H); 8.01 (s; 1H); 7.96 (d; J=7.93 Hz; 1H); 7.86-7.90 (m; 1H); 7.59 (s; 1H); 7.35-7.38 (m; 1H); 7.21 (br s; 2H); 2.53 (s; 3H).

Step 4. A microwave vial was loaded with 5-amino-4-bromo-3-methyl-[2,2′-bipyridine]-6-carboxamide (63 mg, 0.205 mmol), 4-(4,4,5,5-tetramethyl-1,3,2-dioxaborolan-2-yl)-1H-indazole (61 mg, 0.250 mmol), dioxane (2 mL), 2 M aqueous solution of Na₂CO₃ (0.256 mL, 0.512 mmol) and PdCl₂(dppf)·DCM (9 mg, 0.011 mmol). The mixture was degassed and back-filled with argon (3 cycles) and then stirred at 90° C. for 18 h. Upon cooling to rt, additional 4-(4,4,5,5-tetramethyl-1,3,2-dioxaborolan-2-yl)-1H-indazole (61 mg, 0.250 mmol) was added, followed by K₃PO₄ (87 mg, 0.410 mmol), Pd₂(dba)₃ (19 mg, 0.021 mmol) and 1 M THF solution of P(t-Bu)₃ (0.041 mL, 0.041 mmol). The mixture was degassed and back-filled with argon (3 cycles) then stirred at 100° C. for 4 h. Upon cooling to rt, the mixture was diluted with water (20 mL) and EtOAc (25 mL). The mixture was filtered through Celite™, the layers were partitioned and the aqueous layer was extracted with EtOAc. The combined organic layers was washed with brine, dried over MgSO₄, filtered and concentrated in vacuo. The residue was purified by silica gel chromatography eluting with a gradient of EtOAc (40 to 100%) in heptane to provide 5-amino-4-(1H-indazol-4-yl)-3-methyl-[2,2′-bipyridine]-6-carboxamide (49 mg, 72% yield). MS: [M+H]⁺: 345.1; ¹H NMR (400 MHz, DMSO-d6) δ 13.26 (s; 1H); 8.56-8.57 (m; 1H); 8.01-8.05 (m; 2H); 7.86-7.90 (m; 1H); 7.66 (s; 1H); 7.63 (d; J=8.49 Hz; 1H); 7.48-7.53 (m; 1H); 7.46-7.48 (m; 1H); 7.32-7.35 (m; 1H); 6.99 (d; J=6.89 Hz; 1H); 6.25 (s; 2H); 2.04 (s; 3H).

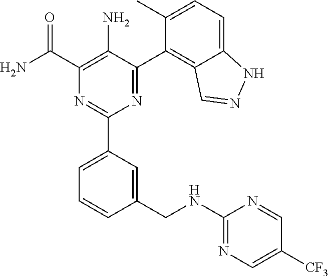 [SMILES: Cc1ccc2[nH]ncc2c1-c1nc(-c2cccc(CNc3ncc(C(F)(F)F)cn3)c2)nc(C(N)=O)c1N]

### Compound 296 (5-amino-4-(5-methyl-1H-indazol-4-yl)-6′-((1-phenylpropyl)amino)-[2,2′-bipyridine]-6-carboxamide)

Step 1. To a solution of Intermediate J (200 mg, 660.7 μmol), (3-(((tert-butoxycarbonyl)amino)methyl)phenyl)boronic acid (325 mg, 1.29 mmol), and Pd(dppf)Cl₂·DCM (55 mg, 67.4 μmol) in dioxane (2.5 mL) was added 2 M aqueous K₂CO₃ solution (1.3 mL, 2.6 mmol). The reaction mixture was stirred at 100° C. for 5 h. The reaction mixture was cooled to rt, poured into water and extracted with EtOAc (3×). The combined organic layers was washed with brine, dried over Na₂SO₄, filtered and concentrated. The residue was purified by silica gel chromatography eluting with a gradient of MeOH (0 to 10%) in DCM to provide 2-[3-[5-amino-4-carbamoyl-6-(5-methyl-1H-indazol-4-yl)pyrimidin-2-yl]phenyl]acetic acid (275 mg, 88% yield). MS: [M+H]⁺: 474.4.

Step 2. To a solution of tert-butyl N-[[3-[5-amino-4-carbamoyl-6-(5-methyl-1H-indazol-4-yl)pyrimidin-2-yl]phenyl]methyl]carbamate (275 mg, 580.75 μmol) in MeOH (3 mL) was added 4 M solution of HCl in dioxane (3 mL, 12 mmol). The reaction mixture was stirred at rt for 1 h. The volatiles were removed under vacuum to yield 5-amino-2-[3-(aminomethyl)phenyl]-6-(5-methyl-1H-indazol-4-yl)pyrimidine-4-carboxamide dihydrochloride (256 mg, 99% yield). MS: [M+H]⁺: 374.3.

Step 3. To a solution of 5-amino-2-[3-(aminomethyl)phenyl]-6-(5-methyl-1H-indazol-4-yl)pyrimidine-4-carboxamide dihydrochloride (50 mg, 112.0 μmol) and 2-chloro-5-(trifluoromethyl)pyrimidine (21 mg, 115.1 μmol) in DMSO (1 mL) was added triethylamine (100 μL, 717.5 μmol). The reaction mixture was stirred at rt for 18 h. The crude reaction mixture was filtered, and the filtrate was purified by preparative HPLC C18 column eluting with ACN/water/0.1% formic acid to provide 5-amino-6-(5-methyl-1H-indazol-4-yl)-2-[3-[[[5-(trifluoromethyl)pyrimidin-2-yl]amino]methyl]phenyl]pyrimidine-4-carboxamide (1.2 mg 2% yield). MS: [M+H]⁺: 520.3.

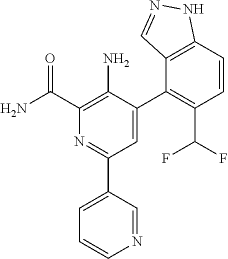 [SMILES: NC(=O)c1nc(-c2cccnc2)cc(-c2c(C(F)F)ccc3[nH]ncc23)c1N]

### Compound 321 (5-amino-4-(5-(difluoromethyl)-1H-indazol-4-yl)-[2,3′-bipyridine]-6-carboxamide)

Step 1. A microwave vial was loaded Intermediate Q (100 mg, 0.310 mmol), Intermediate P (176 mg, 0.465 mmol), KF (55 mg, 0.931 mmol), Pd(PPh₃)₄ (36 mg, 0.031 mmol) and EtOH (2 mL). The vial was sealed, degassed and back-filled with argon (3 cycles). It was heated to 100° C. for 90 min. Upon cooling to rt, the mixture was diluted with water (25 mL) and EtOAc (25 mL). The layers were partitioned and the aqueous layer was extracted with EtOAc (20 mL). The combined organic layers was washed with brine, dried over MgSO₄, filtered and concentrated. The residue was purified by silica gel chromatography eluting with a gradient of EtOAc (0 to 100%) in heptane to provide ethyl 5-amino-4-(5-(difluoromethyl)-1-(tetrahydro-2H-pyran-2-yl)-1H-indazol-4-yl)-[2,3′-bipyridine]-6-carboxylate (45 mg, 31% yield). MS: [M+H]⁺: 494.2.

Step 2. Ammonia (7 N in MeOH, 4 mL, 28 mmol) was added to ethyl 5-amino-4-(5-(difluoromethyl)-1-(tetrahydro-2H-pyran-2-yl)-1H-indazol-4-yl)-[2,3′-bipyridine]-6-carboxylate (45 mg, 0.091 mmol). The reaction mixture was stirred at rt over weekend. The volatiles were removed in vacuo and the residue was dissolved in DCM (1 mL) followed by addition of TFA (0.27 mL, 3.53 mmol). The reaction mixture was stirred at rt for 6 h. Toluene (3 mL) was added and the volatiles were removed in vacuo. The residue was dissolved in EtOAc (20 mL) and aqueous saturated NaHCO₃ solution was added. The layers were partitioned and the aqueous layer was extracted with EtOAc (15 mL). The combined organic layers was washed with brine, dried over MgSO₄, filtered and concentrated to dryness. The residue was dissolved in EtOAc and adsorbed on silica gel. The residue was purified by silica gel chromatography eluting with a gradient of EtOAc (30 to 100%) in heptane to provide 5-amino-4-(5-(difluoromethyl)-1H-indazol-4-yl)-[2,3′-bipyridine]-6-carboxamide (9 mg, 26% yield). MS: [M+H]⁺: 381.1; ¹H NMR (400 MHz, DMSO-d6) δ 13.50 (s; 1H); 9.37 (s; 1H); 8.52-8.56 (m; 2H); 8.44 (s; 1H); 7.93 (s; 1 H); 7.73-7.81 (m; 3H); 7.61 (s; 1H); 7.42 (dd; J=8.01; 4.83 Hz; 1H); 6.80 (t; J=54.86 Hz; 1H); 6.62 (s; 2H).

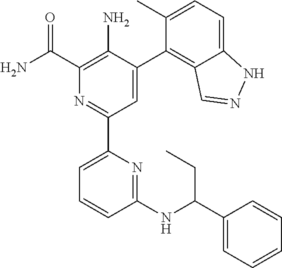 [SMILES: CCC(Nc1cccc(-c2cc(-c3c(C)ccc4[nH]ncc34)c(N)c(C(N)=O)n2)n1)c1ccccc1]

### Compound 331 (5-amino-4-(5-methyl-1H-indazol-4-yl)-6′-((1-phenylpropyl)amino)-[2,2′-bipyridine]-6-carboxamide)

To a solution of Intermediate O (150 mg, 414 μmol) in NMP (3 mL) was added 1-phenylpropan-1-amine (504 mg, 3.73 mmol). The solution was stirred at 200° C. for 3 h. More 1-phenylpropan-1-amine (168 mg, 1.24 mmol) was added and the mixture was stirred 200° C. for 3 h. The crude reaction mixture was filtered and the filtrate was purified by preparative HPLC C18 column eluting with ACN/water/0.1% formic acid to provide 3-amino-4-(5-methyl-1H-indazol-4-yl)-6-[6-(1-phenylpropylamino)-2-pyridyl]pyridine-2-carboxamide as a mixture of 4 isomers (63 mg, 32% yield). MS: [M+H]⁺: 478.2; ¹H NMR (400 MHz, DMSO-d6) δ 13.13 (s, 1H), 8.18 (d, J=35.3 Hz, 1H), 7.99 (d, J=19.7 Hz, 1H), 7.66-7.59 (m, 1H), 7.56 (d, J=8.1 Hz, 1H), 7.48 (s, 1H), 7.45-7.32 (m, 2H), 7.32-7.14 (m, 3H), 7.09 (s, 2H), 7.03-6.85 (m, 3H), 6.38 (s, 2H), 4.83-4.35 (m, 1H), 2.19 (s, 1H), 2.07 (s, 2H), 1.77-1.47 (m, 3H), 0.86-0.65 (m, 3H).

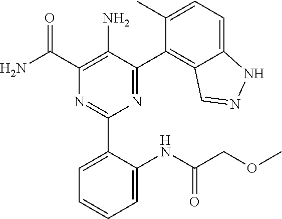 [SMILES: COCC(=O)Nc1ccccc1-c1nc(C(N)=O)c(N)c(-c2c(C)ccc3[nH]ncc23)n1]

### Compound 332 (5-amino-2-(2-(2-methoxyacetamido)phenyl)-6-(5-methyl-1H-indazol-4-yl)pyrimidine-4-carboxamide)

Step 1. To a solution of 2-(4,4,5,5-tetramethyl-1,3,2-dioxaborolan-2-yl)aniline (250 mg, 1.14 mmol) and DIPEA (250 μL, 1.44 mmol) in DCM (5 mL) was added 2-methoxyacetyl chloride (115 μL, 1.26 mmol). The reaction mixture was stirred at rt for 1 h. The volatiles were evaporated and the residue was purified by silica gel chromatography eluting with a gradient of EtOAc (0 to 75%) in heptane to provide 2-methoxy-N-[2-(4,4,5,5-tetramethyl-1,3,2-dioxaborolan-2-yl)phenyl]acetamide (165 mg, 50% yield).

Step 2. To a solution Intermediate J (50 mg, 165.2 μmol), 2-methoxy-N-[2-(4,4,5,5-tetramethyl-1,3,2-dioxaborolan-2-yl)phenyl]acetamide (100 mg, 343.5 μmol) and Pd(dppf)Cl₂·DCM (15 mg, 18.4 μmol) in dioxane (1.5 mL) was added 2 M aqueous solution of K₂CO₃ (330 μL, 0.66 mmol). The reaction mixture was stirred at 100° C. for 5 h. The crude reaction mixture was cooled to rt, filtered and the filtrate was purified by preparative HPLC C18 column eluting with ACN/water/0.1% formic acid to provide 5-amino-2-[2-[(2-methoxyacetyl)amino]phenyl]-6-(5-methyl-1H-indazol-4-yl)pyrimidine-4-carboxamide (15 mg, 20% yield). MS: [M+H]⁺: 432.4; ¹H NMR (400 MHz, DMSO-d6) δ 13.13 (s, 1H), 11.99 (s, 1H), 8.52-8.38 (m, 3H), 7.97 (d, J=2.3 Hz, 1H), 7.65 (s, 1H), 7.62-7.53 (m, 1H), 7.39-7.31 (m, 2H), 7.18-7.11 (m, 1H), 6.49 (s, 2H), 3.73-3.56 (m, 2H), 2.54 (s, 3H), 2.21 (s, 3H).

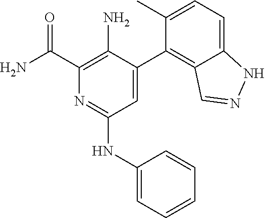 [SMILES: Cc1ccc2[nH]ncc2c1-c1cc(Nc2ccccc2)nc(C(N)=O)c1N]

### Compound 335 (3-amino-4-(5-methyl-1H-indazol-4-yl)-6-(phenylamino)picolinamide)

Step 1. Intermediate K (0.25 g, 0.8285 mmol) was dissolved in DCM (5 mL) and cooled to 0° C. Triethylamine (344 μL, 2.485 mmol) and Boc-anhydride (0.27 g, 1.245 mmol) where added and the reaction mixture was stirred for 5 h at rt. The reaction mixture was quenched with water (20 mL) then extracted with EtOAc (3×20 mL). The combined organic layer was dried over Na₂SO₄, filtered and concentrated. The residue was purified by silica gel chromatography eluting with 20% EtOAc in hexanes to provide tert-butyl 4-(3-amino-2-carbamoyl-6-chloropyridin-4-yl)-5-methyl-1H-indazole-1-carboxylate (0.2 g, 60% yield). MS: [M+H]⁺: 402.2.

Step 2. A pressure vessel was charged with tert-butyl 4-(3-amino-2-carbamoyl-6-chloropyridin-4-yl)-5-methyl-1H-indazole-1-carboxylate (0.2 g, 0.498 mmol), dioxane (3 mL), aniline (41 μL, 4.98 mmol) and Cs₂CO₃ (0.486 g, 1.49 mmol). The reaction mixture was purged with N₂ gas for 10 min. XPhos-Pd-G3 (0.081 g, 0.09951 mmol) was then added and the reaction mixture was heated at 100° C. for 5 h. The reaction mixture was quenched with water (20 mL) and extracted with EtOAc (3×20 mL). The combined organic layer was dried over Na₂SO₄, filtered and concentrated. The residue was purified by preparative HPLC C18 column eluting with ACN/water/0.1% formic acid to provide 3-amino-4-(5-methyl-1H-indazol-4-yl)-6-(phenylamino)picolinamide (20 mg, 11% yield). MS: [M+H]⁺: 359.4; ¹H NMR (400 MHz, DMSO-d6) δ 13.15 (s, 1H), 8.71 (s, 1H), 7.62 (s, 1H), 7.56-7.49 (m, 5H), 7.35 (d, J=8.4 Hz, 1H), 7.27 (t, J=7.6 Hz, 1H), 6.86-6.81 (m, 2H), 5.82 (s, 2H), 2.21 (s, 3H).

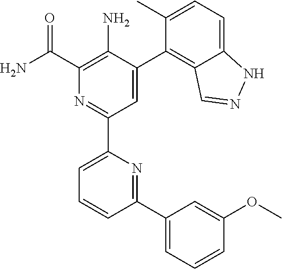 [SMILES: COc1cccc(-c2cccc(-c3cc(-c4c(C)ccc5[nH]ncc45)c(N)c(C(N)=O)n3)n2)c1]

### Compound 340 (5-amino-6′-(3-methoxyphenyl)-4-(5-methyl-1H-indazol-4-yl)-[2,2′-bipyridine]-6-carboxamide)

Step 1. Intermediate O (0.3 g, 0.828 mmol) was dissolved in DMF (5 mL) and treated with NaH (60% dispersion in mineral oil, 0.05 g, 1.24 mmol) at 0° C. and then stirred for 20 min. p-Methoxybenzyl alcohol (0.125 g, 0.91 mmol) was added and the mixture was stirred at 60° C. for 12 h. The cooled reaction mixture was poured into chilled water and the solids were filtered, dried under vacuum and triturated with n-pentane and hexanes to get 5-amino-6′-((4-methoxybenzyl)oxy)-4-(5-methyl-1H-indazol-4-yl)-[2,2′-bipyridine]-6-carboxamide (0.22 g, 55% yield). MS: [M+H]⁺: 481.4.

Step 2. 5-Amino-6′-((4-methoxybenzyl)oxy)-4-(5-methyl-1H-indazol-4-yl)-[2,2′-bipyridine]-6-carboxamide (0.22 g, 0.458 mmol) was dissolved in dichloromethane (5 mL) and treated with TFA (2 mL) at 0° C. then stirred for 1 h at rt. The reaction mixture was quenched with addition of saturated aqueous solution of sodium bicarbonate (10 mL) and extracted with dichloromethane (3×25 mL). The combined organic layer was dried over Na₂SO₄, filtered and concentrated and triturated with n-pentane and hexanes to get 5-amino-6′-hydroxy-4-(5-methyl-1H-indazol-4-yl)-[2,2′-bipyridine]-6-carboxamide (0.125 g, 76% yield) LCMS: MS: [M+H]⁺: 361.3.

Step 3. 5-Amino-6′-hydroxy-4-(5-methyl-1H-indazol-4-yl)-[2,2′-bipyridine]-6-carboxamide (0.125 g, 0.347 mmol) was dissolved in THF (5 mL) and treated with Et₃N (0.105 g, 1.04 mmol) at rt and stirred for 15 min. 1,1,1-trifluoro-N-phenyl-N-((trifluoromethyl)sulfonyl)methanesulfonamid (0.136 g, 0.382 mmol) was added and the mixture was stirred at rt for 4 h. The reaction mixture was poured into chilled water and the solids were filtered, washed with water, dried and triturated with n-pentane and hexanes to get 5′-amino-6′-carbamoyl-4′-(5-methyl-1H-indazol-4-yl)-[2,2′-bipyridin]-6-yl trifluoromethanesulfonate (0.052 g, 30%). MS: [M+H]⁺: 493.4.

Step 4. 5′-Amino-6′-carbamoyl-4′-(5-methyl-1H-indazol-4-yl)-[2,2′-bipyridin]-6-yl trifluoromethanesulfonate (0.05 g, 0.101 mmol) was dissolved in 9:1 dioxane-water (2 mL). (3-Methoxyphenyl)boronic acid (0.021 g, 0.132 mmol) and Cs₂CO₃ (0.067 g, 0.202 mmol) were added and the mixture was degassed for 10 min with N₂ gas, followed by addition of PdCl₂(dppf)·DCM (0.08 g, 0.0101 mmol). The resulting mixture was stirred at 100° C. for 12 h. The cooled reaction mixture was poured into chilled water (20 mL) and extracted with ethyl acetate (3×10 mL). The combined organic layer was dried over Na₂SO₄, filtered and concentrated. The residue was purified by preparative HPLC C18 column eluting with ACN/water/0.1% formic acid to provide 5-amino-6′-(3-methoxyphenyl)-4-(5-methyl-1H-indazol-4-yl)-[2,2′-bipyridine]-6-carboxamide (0.005 g, 11% yield). MS: [M+H]⁺: 451.7; ¹H NMR (400 MHz, DMSO-d6) δ 13.19 (s, 1H), 8.67 (d, J=7.6 Hz, 1H), 8.50 (bs, 1H), 8.30 (s, 1H), 7.98-7.90 (m, 2H), 7.66-7.7.58 (m, 4H), 7.41-7.35 (m, 2H), 6.97 (d, J=6.8 Hz, 1H), 6.66 (bs, 1H), 3.76 (s, 3H), 2.23 (s, 3H).

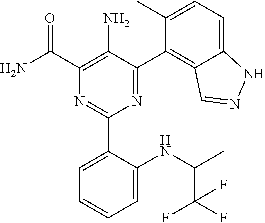 [SMILES: Cc1ccc2[nH]ncc2c1-c1nc(-c2ccccc2NC(C)C(F)(F)F)nc(C(N)=O)c1N]

### Compound 381 (5-amino-6-(5-methyl-1H-indazol-4-yl)-2-(2-((1,1,1-trifluoropropan-2-yl)amino)phenyl)pyrimidine-4-carboxamide)

Step 1. To a solution of 2-bromoaniline (1.01 g, 5.9 mmol) in DCM (10 mL) at 0° C. was added sodium cyanoborohydride (740 mg, 11.8 mmol) followed a slow addition of TFA (10 mL). The resulting foaming mixture was stirred at 0° C. and 1,1,1-trifluoropropan-2-one (1.32 mL, 14.68 mmol) was added slowly. The mixture was allowed to stir at rt overnight. The reaction mixture was concentrated, diluted carefully with aqueous saturated NaHCO₃ solution and extracted with EtOAc (3×). The combined organic layers was washed with brine, dried over Na₂SO₄, filtered and concentrated. The residue was purified by silica gel chromatography eluting with a gradient of EtOAc (0 to 30%) in heptane to provide 2-bromo-N-(2,2,2-trifluoro-1-methyl-ethyl)aniline (985 mg, 63% yield). ¹H NMR (400 MHz, Chloroform-d) δ 7.45 (dd, J=7.9, 1.5 Hz, 1H), 7.25-7.14 (m, 1H), 6.75 (dd, J=8.2, 1.3 Hz, 1H), 6.70-6.57 (m, 1H), 4.49-4.24 (m, 1H), 4.04 (dp, J=8.9, 6.6 Hz, 1H), 1.46 (dd, J=6.8, 0.8 Hz, 3H).

Step 2. A MW vial was charged with 2-bromo-N-(2,2,2-trifluoro-1-methyl-ethyl)aniline (408 mg, 1.52 mmol), dioxane (5 mL), 4,4,5,5-tetramethyl-2-(4,4,5,5-tetramethyl-1,3,2-dioxaborolan-2-yl)-1,3,2-dioxaborolane (503 mg, 1.98 mmol), KOAc (450 mg, 4.59 mmol) and Pd(dppf)Cl₂·DCM (122 mg, 149.4 μmol). The solution was degassed, capped and transferred to a preheated (100° C.) heatblock for 3 h. The reaction mixture was cooled to rt, poured in water and extracted with EtOAc (3×). The combined organic layers was washed with brine, dried over Na₂SO₄, filtered on a silica plug and concentrated. The residue was purified by silica gel chromatography eluting with a gradient of EtOAc (0 to 40%) in heptane to provide 2-(4,4,5,5-tetramethyl-1,3,2-dioxaborolan-2-yl)-N-(2,2,2-trifluoro-1-methyl-ethyl)aniline (312 mg, 65% yield). MS: [M+H]⁺: 316.0.

Step 3. A solution of Intermediate J (150 mg, 495.5 μmol), 2-(4,4,5,5-tetramethyl-1,3,2-dioxaborolan-2-yl)-N-(2,2,2-trifluoro-1-methyl-ethyl)aniline (188 mg, 596.6 μmol) and Pd(dppf)Cl₂·DCM (39 mg, 47.8 μmol) in dioxane (1.5 mL) and 2 M aqueous solution of K₂CO₃ (740 μL, 1.48 mmol) was degassed by bubbling N₂. The reaction mixture was stirred at 100° C. for 2.5 h. The cooled reaction mixture was extracted with DCM (3×) and the combined organic extracts was concentrated. The residue was purified by preparative HPLC C18 column eluting with ACN/water/0.1% formic acid and subsequently repurified by silica gel chromatography eluting with a gradient of EtOAc (20 to 100%) in heptane to provide 5-amino-6-(5-methyl-1H-indazol-4-yl)-2-[2-[(2,2,2-trifluoro-1-methyl-ethyl)amino]phenyl]pyrimidine-4-carboxamide (80 mg, 35% yield). MS: [M+H]⁺: 456.1; ¹H NMR (400 MHz, DMSO-d6) δ 13.16 (dd, J=4.7, 1.6 Hz, 1H), 8.95 (dd, J=49.7, 8.8 Hz, 1H), 8.56 (ddd, J=19.0, 8.0, 1.7 Hz, 1H), 8.45 (dd, J=9.1, 2.4 Hz, 1H), 7.97 (d, J=2.2 Hz, 1H), 7.67 (dt, J=5.5, 1.2 Hz, 1H), 7.61 (d, J=8.5 Hz, 1H), 7.39 (dd, J=8.6, 5.8 Hz, 1H), 7.22 (ddd, J=8.6, 7.1, 1.7 Hz, 1H), 6.91 (d, J=8.4 Hz, 1H), 6.74 (ddd, J=8.1, 7.1, 1.0 Hz, 1H), 6.48 (s, 2H), 4.60-4.36 (m, 1H), 2.25 (d, J=5.3 Hz, 3H), 0.94 (t, J=6.3 Hz, 3H).

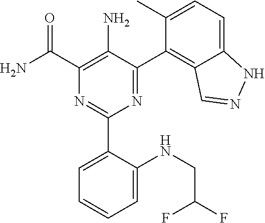 [SMILES: Cc1ccc2[nH]ncc2c1-c1nc(-c2ccccc2NCC(F)F)nc(C(N)=O)c1N]

### Compound 400 (5-amino-2-(2-((2,2-difluoroethyl)amino)phenyl)-6-(5-methyl-1H-indazol-4-yl)pyrimidine-4-carboxamide)

Step 1. A pressure vessel was charged with 2-bromoaniline (512 mg, 2.98 mmol), 2,2-difluoroethyl trifluoromethanesulfonate (640 mg, 2.99 mmol), DCE (5 mL) and DIPEA (1.00 mL, 5.74 mmol). The vessel was sealed and stirred at 80° C. overnight. The cooled reaction mixture was diluted with layers were separated. The aqueous layer was extracted with DCM (3×) and the combined organic extracts was concentrated. The residue was purified by silica gel chromatography eluting with a gradient of EtOAc (0 to 50%) in heptane to provide 2-bromo-N-(2,2-difluoroethyl)aniline (325 mg, 46% yield). MS: [M+H]⁺: 238.0.

Step 2. To a MW vial charged with 2-bromo-N-(2,2-difluoroethyl)aniline (512 mg, 2.17 mmol), 4,4,5,5-tetramethyl-2-(4,4,5,5-tetramethyl-1,3,2-dioxaborolan-2-yl)-1,3,2-dioxaborolane (666 mg, 2.62 mmol) and KOAc (650 mg, 6.62 mmol) was added dioxane (8 mL). The solution was bubbled through with N₂, Pd(dppf)Cl₂·DCM (180 mg, 220.42 μmol) was added. The solution was bubbled through again with N₂, the vial was capped and transferred to a preheated (100° C.) heatblock for 1 h. The cooled reaction mixture was diluted with DCM and adsorbed on silica gel. The residue was purified by silica gel chromatography eluting with a gradient of EtOAc (0 to 100%) in heptane to provide N-(2,2-difluoroethyl)-2-(4,4,5,5-tetramethyl-1,3,2-dioxaborolan-2-yl)aniline (282 mg, 46% yield). MS: [M+H]⁺: 284.1.

Step 3. A solution of Intermediate J (89 mg, 294.0 μmol), N-(2,2-difluoroethyl)-2-(4,4,5,5-tetramethyl-1,3,2-dioxaborolan-2-yl)aniline (139 mg, 333.9 μmol) and Pd(dppf)Cl₂·DCM (30 mg, 36.7 μmol) in dioxane (1.5 mL) and 2 M aqueous solution of K₂CO₃ (441 μL, 882 μmol) was degassed by bubbling N₂. The reaction mixture was stirred at 100° C. for 70 min. The cooled reaction mixture was diluted with water and extracted with DCM (3×). The combined organic extracts was concentrated and the residue was purified by preparative HPLC C18 column eluting with ACN/water/0.1% formic acid to provide 5-amino-2-[2-(2,2-difluoroethylamino)phenyl]-6-(5-methyl-1H-indazol-4-yl)pyrimidine-4-carboxamide (36 mg, 29% yield). MS: [M+H]⁺: 424.2; ¹H NMR (400 MHz, DMSO-d6) δ 13.17 (s, 1H), 8.61 (t, J=6.2 Hz, 1H), 8.45 (dd, J=8.0, 1.7 Hz, 1H), 8.38 (s, 1H), 7.96 (s, 1H), 7.68 (s, 1H), 7.60 (d, J=8.6 Hz, 1H), 7.38 (d, J=8.5 Hz, 1H), 7.22 (td, J=7.7, 7.1, 1.7 Hz, 1H), 6.82 (d, J=8.3 Hz, 1H), 6.71 (t, J=7.5 Hz, 1H), 6.45 (s, 2H), 6.02 (tt, J=55.7, 3.8 Hz, 1H), 3.67-3.45 (m, 2H), 2.26 (s, 3H).

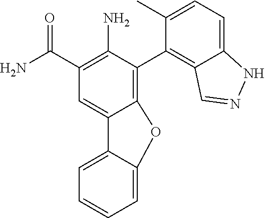 [SMILES: Cc1ccc2[nH]ncc2c1-c1c(N)c(C(N)=O)cc2c1oc1ccccc12]

### Compound 418 (3-amino-4-(5-methyl-1H-indazol-4-yl)dibenzo[b,d]furan-2-carboxamide)

Step 1. A mixture of 5-bromo-6-fluoro-indoline-2,3-dione (4.8 g, 19.7 mmol), (2-hydroxyphenyl)boronic acid (3.0 g, 21.8 mmol), K₂CO₃ (8.15 g, 58.97 mmol), and Pd(dppf)Cl₂ (650 mg, 0.888 mmol) in dioxane (100 mL) was stirred at 80° C. for 4 h. The mixture was cooled down to rt, silica gel was added and the volatiles were evaporated under vacuum. The residue was purified by silica gel chromatography eluting with a gradient of EtOAc (0 to 100%) in heptane to provide 6-fluoro-5-(2-hydroxyphenyl)indoline-2,3-dione (3.1 g, 61% yield). MS: [M−H]−: 256.0.

Step 2. To a solution of 6-fluoro-5-(2-hydroxyphenyl)indoline-2,3-dione (2.0 g, 7.78 mmol) in DMF (30 mL) was added potassium tert-butoxide (1.83 g, 16.3 mmol). The reaction mixture was stirred at rt for 10 min. Water was added and the mixture was filtered to recover 1H-benzofuro[3,2-f]indole-2,3-dione (720 mg, 39% yield). MS: [M−H]−: 236.0.

Step 3. 1H-benzofuro[3,2-f]indole-2,3-dione (140 mg, 590.2 μmol) and 1 M aqueous solution of NaOH (1.0 mL, 1.0 mmol) were heated to 80° C. 30% aqueous solution of hydrogen peroxide (65 μL, 636.3 μmol) was added dropwise. The resulting mixture was stirred at 80° C. for 1 h. The mixture was cooled to rt then 1 M aqueous solution of HCl (1.0 mL, 1.0 mmol) was slowly added until pH 4-5, and the deep orange solid was recovered by filtration. MeOH (10 mL) was added to the filtrate and the resulting suspension was stirred for 15 minutes at 60° C., then sonicated and filtered. The filtrate was concentrated to give 3-aminodibenzofuran-2-carboxylic acid (95 mg, 71% yield) that was used in the next step without further purification. MS: [M+H]⁺: 228.1.

Step 4. To a solution of 3-aminodibenzofuran-2-carboxylic acid (100 mg, 440.1 μmol) in DMF (2 mL) was added ammonium chloride (120 mg, 2.24 mmol), HATU (210 mg, 552.3 μmol) and DIPEA (540 μL, 3.10 mmol). The reaction mixture was stirred at rt for 30 min. Water (6 mL) was added and the suspension was filtered to yield 3-aminodibenzofuran-2-carboxamide (75 mg, 75% yield). MS: [M+H]⁺: 227.1.

Step 5. To a solution of 3-aminodibenzofuran-2-carboxamide (50 mg, 221.0 μmol) in DMF (1 mL) was added NBS (40 mg, 224.7 μmol). The reaction mixture was stirred at rt for 30 min. Water was added and the solid was recover by filtration, washed with water, and dried under air flow to yield 3-amino-4-bromo-dibenzofuran-2-carboxamide (55 mg, 82% yield). MS: [M+H]⁺: 307.0.

Step 6. To a solution of 3-amino-4-bromo-dibenzofuran-2-carboxamide (50 mg, 163.9 μmol) in dioxane (2 mL) were added (5-methyl-1H-indazol-4-yl)boronic acid (58 mg, 329.6 μmol), Pd₂(dba)₃ (15 mg, 16.4 μmol) and 2 M aqueous solution of K₃PO₄ (250 μL, 500 μmol). The mixture was degassed in vacuo and then back-filled with N₂. Tri-tert-buytlphosphonium tetrafluoroborate (10 mg, 34.5 μmol) was added and the mixture was degassed again and then stirred at 95° C. for 1 h. The mixture was cooled to rt, filtered and the filtrate was purified by preparative HPLC C18 column eluting with ACN/water/0.1% formic acid to provide 3-amino-4-(5-methyl-1H-indazol-4-yl)dibenzofuran-2-carboxamide (13 mg, 22% yield). MS: [M+H]⁺: 357.2.

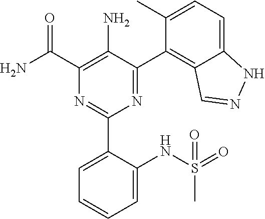 [SMILES: Cc1ccc2[nH]ncc2c1-c1nc(-c2ccccc2NS(C)(=O)=O)nc(C(N)=O)c1N]

### Compound 427 (5-amino-6-(5-methyl-1H-indazol-4-yl)-2-(2-(methylsulfonamido)phenyl)pyrimidine-4-carboxamide)

To a solution of Intermediate T (40 mg, 111.3 μmol) in DCM (1 mL) were added pyridine (90 μL, 1.11 mmol) and methanesulfonyl chloride (35 μL, 452.2 μmol). The mixture was stirred at rt for 1 h. The volatiles were removed in vacuo. Water was added to the residue and the mixture was stirred at rt for for 20 min and then filtered. The solid was washed with water and dried in vacuo. The crude product was dissolved in MeOH (1 mL) and 0.5 M MeOH solution of sodium methoxide (500 μL, 250 μmol) was added to the mixture. After stirring at rt for for 20 min, the reaction mixture was neutralized with acetic acid. The crude mixture was concentrated to dryness and purified by preparative HPLC C18 column eluting with ACN/water/0.1% formic acid to provide 5-amino-2-[2-(methanesulfonamido)phenyl]-6-(5-methyl-1H-indazol-4-yl)pyrimidine-4-carboxamide (23 mg, 47% yield). MS: [M+H]⁺: 438.3; ¹H NMR (400 MHz, DMSO-d6) δ 13.14 (s, 1H), 11.69 (s, 1H), 8.63 (m, 1H), 8.59-8.40 (m, 1H), 8.12-7.91 (m, 1H), 7.70 (s, 1H), 7.58 (d, J=8.6 Hz, 1H), 7.52-7.45 (m, 1H), 7.44-7.26 (m, 2H), 7.25-7.08 (m, 1H), 6.59 (s, 2H), 2.92 (s, 3H), 2.24 (s, 3H).

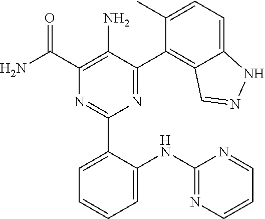 [SMILES: Cc1ccc2[nH]ncc2c1-c1nc(-c2ccccc2Nc2ncccn2)nc(C(N)=O)c1N]

### Compound 449 (5-amino-6-(5-methyl-1H-indazol-4-yl)-2-(2-(pyrimidin-2-ylamino)phenyl)pyrimidine-4-carboxamide)

To a solution of Intermediate T (30 mg, 83.5 μmol) in 2,2,2-trifluoroethanol (1 mL) were added 2-fluoropyrimidine (7 μL, 87.1 μmol) and TFA (13 μL, 168.7 μmol). The mixture was stirred at 110° C. for 0.5 h in microwave. The volatiles were evaporated and the residue was purified by preparative HPLC C18 column eluting with ACN/water/0.1% formic acid to provide 5-amino-6-(5-methyl-1H-indazol-4-yl)-2-[2-(pyrimidin-2-ylamino)phenyl]pyrimidine-4-carboxamide (15 mg, 41% yield). MS: [M+H]⁺: 438.2; ¹H NMR (400 MHz, DMSO-d6) δ 13.14 (s, 1H), 12.01 (s, 1H), 8.55 (dd, J=8.4, 1.2 Hz, 1H), 8.46-8.34 (m, 4H), 8.03 (s, 1H), 7.66 (s, 1H), 7.60 (d, J=8.6 Hz, 1H), 7.36 (m, 2H), 7.03 (m, 1H), 6.84 (t, J=4.8 Hz, 1H), 6.54 (s, 2H), 2.25 (s, 3H).

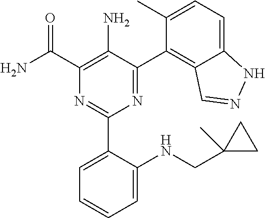 [SMILES: Cc1ccc2[nH]ncc2c1-c1nc(-c2ccccc2NCC2(C)CC2)nc(C(N)=O)c1N]

### Compound 452 (5-amino-6-(5-methyl-1H-indazol-4-yl)-2-(2-(pyrimidin-2-ylamino)phenyl)pyrimidine-4-carboxamide)

Step 1. To a suspension of Intermediate S (497 mg, 1.65 mmol) in THF (5 mL) was added 1 M THF solution of borane (11.6 mL, 11.6 mmol). The mixture was heated to reflux for 3 h. MeOH was slowly added to the cooled reaction mixture and the volatiles were evaporated. The residue was purified by silica gel chromatography eluting with a gradient of EtOAc (0 to 50%) in heptane to provide N-[(1-methylcyclopropyl)methyl]-2-(4,4,5,5-tetramethyl-1,3,2-dioxaborolan-2-yl)aniline (100 mg, 21% yield). MS: [M+H]⁺: 288.1.

Step 2. A MW vial was charged with Intermediate J (78 mg, 257.7 μmol), Pd(dppf)Cl₂·DCM (21 mg, 25.7 μmol), N-[(1-methylcyclopropyl)methyl]-2-(4,4,5,5-tetramethyl-1,3,2-dioxaborolan-2-yl)aniline (100 mg, 348.2 μmol) and dioxane (2 mL). 2 M aqueous solution of K₂CO₃ (390 μL, 0.78 mmol) was added, N₂ was bubbled through the mixture of 10 min and the vial was capped and transferred to a preheated (100° C.) heat block for 135 min. The cooled reaction mixture was diluted with water and extracted with DCM (3×). The combined extracts was concentrated and the residue was purified by preparative HPLC C18 column eluting with ACN/water/0.1% formic acid to provide 5-amino-2-[2-[(1-methylcyclopropyl)methylamino]phenyl]-6-(5-methyl-1H-indazol-4-yl)pyrimidine-4-carboxamide (38 mg, 35% yield). MS: [M+H]⁺: 428.3; ¹H NMR (400 MHz, DMSO-d6) δ 13.13 (br s, 1H), 8.77 (t, J=4.4 Hz, 1H), 8.54 (dd, J=8.0, 1.7 Hz, 1H), 8.48 (d, J=2.4 Hz, 1H), 7.95 (d, J=2.4 Hz, 1H), 7.64 (d, J=1.0 Hz, 1H), 7.59 (dd, J=8.5, 1.0 Hz, 1H), 7.37 (d, J=8.6 Hz, 1H), 7.16 (ddd, J=8.6, 7.1, 1.7 Hz, 1H), 6.61 (ddd, J=8.1, 7.1, 1.1 Hz, 1H), 6.52 (dd, J=8.4, 1.2 Hz, 1H), 6.42 (br s, 2H), 2.78 (qd, J=11.8, 4.3 Hz, 2H), 2.25 (s, 3H), 0.52 (s, 3H), 0.17-0.07 (m, 1H), 0.06-−0.06 (m, 1H), −0.18-−0.28 (m, 1H), −0.35 (dt, J=9.2, 4.7 Hz 1H).

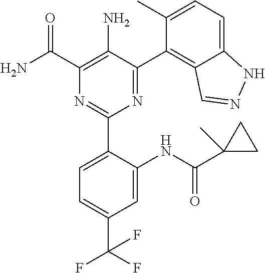 [SMILES: Cc1ccc2[nH]ncc2c1-c1nc(-c2ccc(C(F)(F)F)cc2NC(=O)C2(C)CC2)nc(C(N)=O)c1N]

### Compound 454 (5-amino-6-(5-methyl-1H-indazol-4-yl)-2-(2-(1-methylcyclopropane-1-carboxamido)-4-(trifluoromethyl)phenyl)pyrimidine-4-carboxamide)

Step 1. To a solution of Intermediate J (60 mg, 198.2 μmol) in dioxane (1 mL) were added 2-(4,4,5,5-tetramethyl-1,3,2-dioxaborolan-2-yl)-5-(trifluoromethyl)aniline (85 mg, 296.1 μmol), 2 M aqueous solution of K₂CO₃ (340 μL, 0.68 mmol) and Pd(dppf)Cl₂·DCM (12 mg, 16.40 μmol). The mixture was degassed in vacuo, back-filled with N₂ and stirred at 110° C. for 0.5 h. The cooled reaction mixture was diluted with water and extracted with EtOAc (3×15 mL). The combined organic extracts were washed with water and brine consecutively, dried over sodium sulfate, filtered and concentrated. The residue was purified by silica gel chromatography eluting with a gradient of EtOAc (50 to 100%) in heptane to provide 5-amino-2-[2-amino-4-(trifluoromethyl)phenyl]-6-(5-methyl-1H-indazol-4-yl)pyrimidine-4-carboxamide (61 mg, 72% yield). MS: [M+H]⁺: 428.2.

Step 2. To a solution of 5-amino-2-[2-amino-4-(trifluoromethyl)phenyl]-6-(5-methyl-1H-indazol-4-yl)pyrimidine-4-carboxamide (30 mg, 70.2 μmol) in DCM (1 mL) were added pyridine (26 μL, 321.5 μmol) and 1-methylcyclopropanecarbonyl chloride (9 μL, 91.1 μmol). The mixture was stirred at rt for 10 min then the volatiles were removed in vacuo. Water was added and the mixture was stirred at rt for 20 min then filtered. The solid was washed with water, dried in vacuo. The solid was dissolved in MeOH (1 mL) and to the mixture was added 0.5 M MeOH solution of sodium methoxide (280 μL 0.14 mmol). After stirring at rt for for 10 min, the reaction mixture was neutralized with acetic acid and concentrated. The residue was purified by preparative HPLC C18 column eluting with ACN/water/0.1% formic acid to provide 5-amino-2-[2-[(1-methylcyclopropanecarbonyl)amino]-4-(trifluoromethyl)phenyl]-6-(5-methyl-1H-indazol-4-yl)pyrimidine-4-carboxamide (20 mg, 56% yield). MS: [M+H]⁺: 510.3; ¹H NMR (400 MHz, DMSO-d6) δ 13.14 (s, 1H), 12.28 (s, 1H), 8.89 (d, J=2.3 Hz, 1H), 8.78-8.70 (m, 1H), 8.64 (d, J=8.8 Hz, 1H), 7.98 (s, 1H), 7.79-7.64 (m, 2H), 7.58 (d, J=8.6 Hz, 1H), 7.34 (d, J=8.7 Hz, 1H), 6.67 (s, 2H), 2.22 (s, 3H), 0.81-0.68 (m, 2H), 0.42 (s, 3H), 0.27-0.09 (m, 2H).

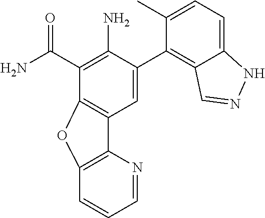 [SMILES: Cc1ccc2[nH]ncc2c1-c1cc2c(oc3cccnc32)c(C(N)=O)c1N]

### Compound 458 (7-amino-8-(5-methyl-1H-indazol-4-yl)benzofuro[3,2-b]pyridine-6-carboxamide)

Step 1. To a solution of 6-amino-3-bromo-2-fluorobenzonitrile (3.0 g, 14.0 mmol) in toluene (30 mL) was added p-toluenesulfonic acid monohydrate (130 mg, 0.683 mmol) and 2,5-hexanedione (2 mL, 17.0 mmol). The reaction mixture was refluxed using a Dean-Stark apparatus for 2 h. Upon cooling to rt, the mixture was diluted with EtOAc (50 mL) and aqueous saturated NaHCO₃ solution, the layers were separated and the aqueous layer was extracted with EtOAc (40 mL). The combined organic layers was washed with brine and dried over MgSO₄, filtered and concentrated in vacuo. The residue was purified by silica gel chromatography eluting with a gradient of EtOAc (0 to 50%) in hexanes to provide 3-bromo-6-(2,5-dimethyl-1H-pyrrol-1-yl)-2-fluorobenzonitrile (3.2 g, 78% yield). MS: [M+H]⁺: 293.0/295.0.

Step 2. To a solution of 3-bromo-6-(2,5-dimethyl-1H-pyrrol-1-yl)-2-fluorobenzonitrile (800 mg, 2.73 mmol) in DMF (8 mL) was added Intermediate V (1500 mg, 2.92 mmol), CuI (104 mg, 0.546 mmol), LiCl (238 mg, 5.484 mmol) and Pd(dppf)Cl₂·DCM (111 mg, 0.136 mmol). The mixture was degassed (3 cycles of vacuum/nitrogen) and stirred at 100° C. for 18 h. The cooled reaction mixture was diluted with EtOAc and water. The mixture was filtered through Celite™ and the layers were separated. The aqueous layer was extracted with EtOAc. The combined organic layers was washed with water (2×), brine, dried over MgSO₄, filtered and concentrated. The residue was purified by silica gel chromatography eluting with a gradient of EtOAc (0 to 50%) in hexanes to provide 7-(2,5-dimethyl-1H-pyrrol-1-yl)benzofuro[3,2-b]pyridine-6-carbonitrile (130 mg, 17% yield). MS: [M+H]⁺: 288.1; ¹H-NMR (400 MHz, CDCl3): δ 8.79 (d; J=4.73 Hz; 1H); 8.55 (d; J=8.21 Hz; 1H); 8.04 (d; J=8.44 Hz; 1H); 7.54 (dd; J=8.48; 4.80 Hz; 1H); 7.42 (d; J=8.19 Hz; 1H); 6.01 (s; 2H); 2.08 (s; 6H).

Step 3. A mixture of 7-(2,5-dimethyl-1H-pyrrol-1-yl)benzofuro[3,2-b]pyridine-6-carbonitrile (120 mg, 0.418 mmol), KOH (117 mg, 2.085 mmol) and t-BuOH (5 mL) was heated to 80° C. for 90 min. Upon cooling to rt, the volatiles were removed in vacuo. The residue was diluted with EtOAc and water, the layers were separated and the aqueous layer was extracted with EtOAc. The combined organic layers was washed with brine, dried over MgSO₄, filtered and concentrated afford 7-(2,5-dimethyl-1H-pyrrol-1-yl)benzofuro[3,2-b]pyridine-6-carboxamide (108 mg, 85% yield) which was used as such in the subsequent step without further purification. MS: [M+H]⁺: 306.1

Step 4. A mixture of 7-(2,5-dimethyl-1H-pyrrol-1-yl)benzofuro[3,2-b]pyridine-6-carboxamide (100 mg, 0.328 mmol), hydroxylamine hydrochloride (683 mg, 9.83 mmol), Et₃N (0.365 mL, 2.62 mmol), EtOH (3.75 mL) and water (1.25 mL) was heated to reflux for 18 h. Upon cooling to rt, the volatiles were removed in vacuo. The residue was diluted with EtOAc and water, the layers were separated and the aqueous layer was extracted with EtOAc. The combined organic layers was washed with brine, dried over MgSO₄, filtered and concentrated to dryness to afford 7-aminobenzofuro[3,2-b]pyridine-6-carboxamide (70 mg, 94% yield) which was used as such in the subsequent step without further purification. MS: [M+H]⁺: 228.1.

Step 5. To a solution of 7-aminobenzofuro[3,2-b]pyridine-6-carboxamide (70 mg, 0.308 mmol) in DMF (3 mL) was added NBS (55 mg, 0.309 mmol). The reaction mixture was stirred at rt for 1 h. The mixture was diluted with 10% Na₂S₂O₃ in water and EtOAc. The layers were partitioned and the aqueous layer was extracted with EtOAc. The combined organic layers was washed with brine, dried over MgSO₄, filtered and concentrated in vacuo. The residue was purified by silica gel chromatography eluting with a gradient of EtOAc (30 to 100%) in hexanes to provide 7-amino-8-bromobenzofuro[3,2-b]pyridine-6-carboxamide (20 mg, 21% yield). MS: [M+H]⁺: 306.0/308.0.

Step 6. A mixture of 7-amino-8-bromobenzofuro[3,2-b]pyridine-6-carboxamide (15 mg, 0.049 mmol), (5-methyl-1H-indazol-4-yl)boronic acid (17 mg, 0.097 mmol), 2 M aqueous solution of K₃PO₄ (0.074 mL, 0.148 mmol), Pd₂(dba)₃ (5 mg, 0.005 mmol), tri-tert-butylphosphonium tetrafluoroborate (3 mg, 0.010 mmol) and dioxane (2 mL) was degassed (3 cycles of vacuum/nitrogen). The mixture was heated to reflux for 90 minutes. (5-methyl-1H-indazol-4-yl)boronic acid (17 mg, 0.097 mmol), 2 M aqueous solution of Na₂CO₃ (0.074 mL, 0.148 mmol), Pd₂(dba)₃ (5 mg, 0.005 mmol),), tri-tert-butylphosphonium tetrafluoroborate (3 mg, 0.010 mmol) were added. The mixture was degassed (3 cycles of vacuum/nitrogen atmosphere) and heated to reflux for 90 minutes. The cooled reaction mixture was diluted with water (20 mL) and EtOAc (20 ml) and filtered through Celite™. The filtrate was partitioned and the aqueous layer was extracted with EtOAc (20 mL). The combined organic layers was washed with water and brine, dried over MgSO₄, filtered and concentrated in vacuo. The residue was purified by silica gel chromatography eluting with a gradient of EtOAc (40 to 100%) in hexanes to provide 7-amino-8-(5-methyl-1H-indazol-4-yl)benzofuro[3,2-b]pyridine-6-carboxamide (5.6 mg, 32% yield). MS: [M+H]⁺: 358.1; ¹H-NMR (400 MHz, DMSO): δ 13.09 (s; 1H); 8.48 (d; J=4.82 Hz; 1H); 8.06 (d; J=8.29 Hz; 1H); 7.92 (bs; 2H); 7.71 (s; 1H); 7.51-7.53 (m; 2H); 7.35-7.37 (m; 2H); 6.64 (bs; 2H); 2.18 (s; 3H).

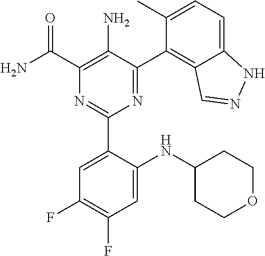 [SMILES: Cc1ccc2[nH]ncc2c1-c1nc(-c2cc(F)c(F)cc2NC2CCOCC2)nc(C(N)=O)c1N]

### Compound 466 (5-amino-2-(4,5-difluoro-2-((tetrahydro-2H-pyran-4-yl)amino)phenyl)-6-(5-methyl-1H-indazol-4-yl)pyrimidine-4-carboxamide)

Step 1. To a solution of 4,5-difluoro-2-(4,4,5,5-tetramethyl-1,3,2-dioxaborolan-2-yl)aniline (196 mg, 768.4 μmol) and tetrahydropyran-4-one (107 μL, 1.15 mmol) in DCM (4 mL) was added acetic acid (70 μL, 1.22 mmol) followed by sodium triacetoxyborohydride (493 mg, 2.33 mmol). The mixture was stirred at rt for 2 h then quenched by the slow addition of saturated NaHCO₃ aqueous solution. The layers were separated and the aqueous layer was extracted with DCM (2×). The combined organic extracts was concentrated and the residue was purified by silica gel chromatography eluting with a gradient of EtOAc (0 to 50%) in heptane to provide N-[4,5-difluoro-2-(4,4,5,5-tetramethyl-1,3,2-dioxaborolan-2-yl)phenyl]tetrahydropyran-4-amine (138 mg, 53% yield). MS: [M+H]⁺: 340.2.

Step 2. A MW vial was charged Intermediate J (107 mg, 353.5 μmol), Pd(dppf)Cl₂·DCM (28 mg, 34.3 μmol), N-[4,5-difluoro-2-(4,4,5,5-tetramethyl-1,3,2-dioxaborolan-2-yl)phenyl]tetrahydropyran-4-amine (138 mg, 406.9 μmol) and dioxane (1 mL). 2 M aqueous solution of K₂CO₃ (530 μL, 1.06 mmol) was added, N₂ was bubbled through the mixture for 10 min, the vial was capped and transferred to a preheated (100° C.) heat block for 2 h. The cooled reaction mixture was diluted with water and extracted with DCM (3×). The combined extracts was concentrated and the residue was purified by preparative HPLC C18 column eluting with ACN/water/0.1% formic acid to provide 5-amino-2-[4,5-difluoro-2-(tetrahydropyran-4-ylamino)phenyl]-6-(5-methyl-1H-indazol-4-yl)pyrimidine-4-carboxamide (50 mg, 30% yield). MS: [M+H]⁺: 480.3; ¹H NMR (400 MHz, DMSO-d6) 13.17 (s, 1H), 8.94 (br d, J=7.7 Hz, 1H), 8.73 (br d, J=2.1 Hz, 1H), 8.69 (dd, J=13.4, 9.9 Hz, 1H), 7.91 (br s, 1H), 7.68 (s, 1H), 7.61 (d, J=8.6 Hz, 1H), 7.39 (d, J=8.6 Hz, 1H), 6.71 (dd, J=14.2, 7.1 Hz, 1H), 6.54 (s, 2H), 3.51 (qt, J=8.2, 3.9 Hz, 1H), 3.45-3.29 (m, 2H), 3.25 (ddd, J=11.6, 9.1, 2.8 Hz, 1H), 3.17 (ddd, J=11.7, 9.1, 2.8 Hz, 1H), 2.26 (s, 3H), 1.78-1.60 (m, 2H), 1.05-0.78 (m, 2H).

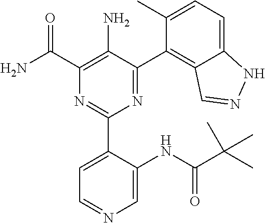 [SMILES: Cc1ccc2[nH]ncc2c1-c1nc(-c2ccncc2NC(=O)C(C)(C)C)nc(C(N)=O)c1N]

Compound 483 (5-amino-6-(5-methyl-1H-indazol-4-yl)-2-(3-pivalamidopyridin-4-yl)pyrimidine-4-carboxamide) A solution of Intermediate J (200 mg, 660.7 μmol) in DMF (5 mL) was bubbled through with N₂ gas. Copper(I) iodide (13 mg, 68.3 μmol), LiCl (58 mg, 1.37 mmol) and Pd(dppf)Cl₂·DCM (52 mg, 66.1 μmol) were added, followed by 2,2-dimethyl-N-(4-tributylstannyl-3-pyridyl)propanamide (500 mg, 1.07 mmol). The reaction mixture was bubbled through with N₂ gas again then stirred at 120° C. for 4 h. Water was added dropwise to the cooled mixture and the brown solid that was recovered by filtration was purified by preparative HPLC C18 column eluting with ACN/water/0.1% formic acid to provide 5-amino-2-[3-(2,2-dimethylpropanoylamino)-4-pyridyl]-6-(5-methyl-1H-indazol-4-yl)pyrimidine-4-carboxamide (10 mg, 3%). MS: [M+H]⁺: 445.3; ¹H NMR (400 MHz, DMSO-d6) δ 13.14 (s, 1H), 11.95 (s, 1H), 9.68 (s, 1H), 8.72 (d, J=9.9 Hz, 2H), 8.40 (d, J=51.5 Hz, 1H), 8.07-7.96 (m, 1H), 7.70 (s, 1H), 7.61 (d, J=8.6 Hz, 1H), 7.35 (d, J=8.6 Hz, 1H), 6.87 (s, 2H), 2.19 (s, 3H), 0.54 (s, 9H).

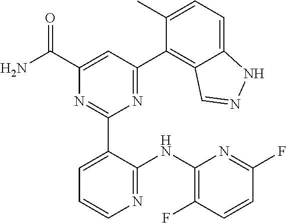 [SMILES: Cc1ccc2[nH]ncc2c1-c1cc(C(N)=O)nc(-c2cccnc2Nc2nc(F)ccc2F)n1]

### Compound 499 (2-(2-((3,6-difluoropyridin-2-yl)amino)pyridin-3-yl)-6-(5-methyl-1H-indazol-4-yl)pyrimidine-4-carboxamide)

Step 1. A mixture of methyl 2,6-dichloropyrimidine-4-carboxylate (4.0 g, 19.32 mmol), 5-methyl-1-tetrahydropyran-2-yl-4-(4,4,5,5-tetramethyl-1,3,2-dioxaborolan-2-yl)indazole (7.0 g, 20.45 mmol), and 2 M aqueous solution of K₃PO₄ (15.5 mL, 31.0 mmol) and PdCl₂(dtbpf) (1.26 g, 1.93 mmol) in dioxane (100 mL) was bubbled through with N₂, then heated at 80° C. for 2 h. The cooled reaction mixture was diluted with water and EtOAc and the mixture was filtered through Celite™. The aqueous layer was extracted twice with EtOAc and the combined organic layers was washed with brine, dried over Na₂SO₄, filtered and concentrated. The residue was purified by silica gel chromatography eluting with a gradient of EtOAc (0 to 75%) in heptane to provide methyl 2-chloro-6-(5-methyl-1-tetrahydropyran-2-yl-indazol-4-yl)pyrimidine-4-carboxylate (3.75 g, 50% yield). MS: [M−THP+H]⁺: 303.1/305.1.

Step 2. A RBF was loaded with methyl 2-chloro-6-(5-methyl-1-tetrahydropyran-2-yl-indazol-4-yl)pyrimidine-4-carboxylate (1.15 g, 2.97 mmol), 3-(4,4,5,5-tetramethyl-1,3,2-dioxaborolan-2-yl)pyridin-2-amine (1.0 g, 4.54 mmol) in dioxane (50 mL) and 2 M aqueous solution of K₂CO₃ (3.7 mL, 7.4 mmol). The mixture was bubbled through with N₂. PdCl₂(dtbpf) (200 mg, 306.9 μmol) was added. The mixture was bubbled through again with N₂ and stirred at 110° C. for 1 h. The reaction mixture was cooled to rt, poured in water and extracted with EtOAc (3×). The combined organic layers were washed with brine, dried over Na₂SO₄, filtered and concentrated. The residue was purified by silica gel chromatography eluting with a gradient of EtOAc (0 to 100%) in heptane to provide methyl 2-(2-amino-3-pyridyl)-6-(5-methyl-1-tetrahydropyran-2-yl-indazol-4-yl)pyrimidine-4-carboxylate (1.1 g, 83% yield). MS: [M+H]⁺: 445.3.

Step 3. To a solution of methyl 2-(2-amino-3-pyridyl)-6-(5-methyl-1-tetrahydropyran-2-yl-indazol-4-yl)pyrimidine-4-carboxylate (485 mg, 1.09 mmol) in toluene (10 mL) were added 2-bromo-3,6-difluoro-pyridine (275 mg, 1.42 mmol), Pd(OAc)₂ (40 mg, 178.2 μmol), Cs₂CO₃ (900 mg, 2.76 mmol) and Xantphos (160 mg, 276.7 μmol). The mixture was degassed in vacuo, back-filled with N₂ and stirred at 110° C. for 1 h. The cooled reaction mixture was diluted with DCM, filtered through Celite™ and concentrated. The residue was purified by silica gel chromatography eluting with a gradient of EtOAc (20 to 100%) in heptane to provide methyl 2-[2-[(3,6-difluoro-2-pyridyl)amino]-3-pyridyl]-6-(5-methyl-1-tetrahydropyran-2-yl-indazol-4-yl)pyrimidine-4-carboxylate (325 mg, 53% yield). MS: [M+H]⁺: 558.2.

Step 4. A MW vessel was charged with 7 N ammonia solution in MeOH (6 mL, 42 mmol) and methyl 2-[2-[(3,6-difluoro-2-pyridyl)amino]-3-pyridyl]-6-(5-methyl-1-tetrahydropyran-2-yl-indazol-4-yl)pyrimidine-4-carboxylate (225 mg, 403.6 μmol). The vessel was sealed and heated 80° C. for 1 h then concentrated. The residue was dissolved in 4 M dioxane solution of HCl (3 mL, 12 mmol) and MeOH (3 mL) and the solution was stirred at 55° C. for 30 min in a sealed microwave vial. The volatiles were evaporated to dryness and residue was taken in 1 mL of DCM. A few drops of Et₃N were added until pH was slightly basic and the mixture was evaporated to dryness again. The solid was purified by preparative HPLC C18 column eluting with ACN/water/0.1% formic acid to provide 2-[2-[(3,6-difluoro-2-pyridyl)amino]-3-pyridyl]-6-(5-methyl-1H-indazol-4-yl)pyrimidine-4-carboxamide (73 mg, 39% yield). MS: [M+H]⁺: 459.3; ¹H NMR (400 MHz, DMSO-d6) δ 13.22 (s, 1H), 11.47 (s, 1H), 9.25 (dd, J=7.9, 1.9 Hz, 1H), 8.84-8.66 (m, 1H), 8.37 (dd, J=4.7, 1.9 Hz, 1H), 8.23-8.09 (m, 1H), 8.06 (s, 1H), 7.92 (s, 1H), 7.77 (td, J=8.9, 6.4 Hz, 1H), 7.63 (dd, J=8.4, 0.9 Hz, 1H), 7.39 (d, J=8.6 Hz, 1H), 7.15 (dd, J=7.9, 4.7 Hz, 1H), 6.80 (ddd, J=8.6, 3.5, 2.3 Hz, 1H), 2.46 (s, 3H).

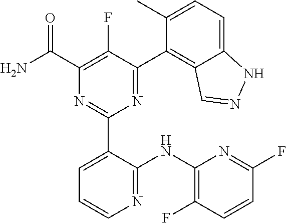 [SMILES: Cc1ccc2[nH]ncc2c1-c1nc(-c2cccnc2Nc2nc(F)ccc2F)nc(C(N)=O)c1F]

### Compound 500 (2-(2-((3,6-difluoropyridin-2-yl)amino)pyridin-3-yl)-5-fluoro-6-(5-methyl-1H-indazol-4-yl)pyrimidine-4-carboxamide)

Step 1. A mixture of ethyl 2,6-dichloro-5-fluoro-pyrimidine-4-carboxylate (1.0 g, 4.18 mmol), 5-methyl-1-tetrahydropyran-2-yl-4-(4,4,5,5-tetramethyl-1,3,2-dioxaborolan-2-yl)indazole (1.5 g, 4.38 mmol), 2 M aqueous solution of K₃PO₄ (3.3 mL, 6.6 mmol) and PdCl₂(dtbpf) (270 mg, 414.3 μmol) in dioxane (30 mL) was bubbled through with N₂, then heated at 140° C. for 2 h in under microwaves. The cooled reaction mixture was diluted with water and EtOAc and filtered through Celite™. The aqueous layer was extracted twice with EtOAc. The combined organic layers was washed with brine, dried over Na₂SO₄, filtered and concentrated. The residue was purified by silica gel chromatography eluting with a gradient of EtOAc (0 to 100%) in heptane to provide ethyl 2-chloro-5-fluoro-6-(5-methyl-1-tetrahydropyran-2-yl-indazol-4-yl)pyrimidine-4-carboxylate (350 mg, 20% yield). MS: [M+H]⁺: 419.3.

Step 2. A RBF was loaded with ethyl 2-chloro-5-fluoro-6-(5-methyl-1-tetrahydropyran-2-yl-indazol-4-yl)pyrimidine-4-carboxylate (350 mg, 835.6 μmol), 3-(4,4,5,5-tetramethyl-1,3,2-dioxaborolan-2-yl)pyridin-2-amine (280 mg, 1.27 mmol) in dioxane (3 mL) and 2 M aqueous solution of K₂CO₃ (1.1 mL, 2.2 mmol). The mixture was bubbled through with N₂ and PdCl₂(dtbpf) (57 mg, 87.5 μmol) was added. The mixture was bubbled through with N₂ again and then stirred at 110° C. for 1 h. The reaction mixture was cooled to rt, poured into water and extracted with EtOAc (3×). The combined organic layers was washed with brine, dried over Na₂SO₄, filtered and concentrated. The residue was purified by silica gel chromatography eluting with a gradient of EtOAc (0 to 100%) in heptane to provide ethyl 2-(2-amino-3-pyridyl)-5-fluoro-6-(5-methyl-1-tetrahydropyran-2-yl-indazol-4-yl)pyrimidine-4-carboxylate (310 mg, 78% yield). MS: [M+H]⁺: 419.3.

Step 3. To a solution of ethyl 2-(2-amino-3-pyridyl)-5-fluoro-6-(5-methyl-1-tetrahydropyran-2-yl-indazol-4-yl)pyrimidine-4-carboxylate (100 mg, 209.9 μmol) in toluene (2 mL) were added 2-bromo-3,6-difluoro-pyridine (60 mg, 309.3 μmol), Pd(OAc)₂ (8 mg, 35.6 μmol), Cs₂CO₃ (175 mg, 537.11 μmol) and Xantphos (31 mg, 53.58 μmol). The mixture was degassed in vacuo, back-filled with N₂ then stirred at 110° C. for 1 h. The cooled reaction mixture was diluted with DCM, filtered through Celite™ and concentrated. The residue was purified by silica gel chromatography eluting with a gradient of EtOAc (20 to 100%) in heptane to provide ethyl 2-[2-[(3,6-difluoro-2-pyridyl)amino]-3-pyridyl]-5-fluoro-6-(5-methyl-1-tetrahydropyran-2-yl-indazol-4-yl)pyrimidine-4-carboxylate (100 mg, 81% yield). MS: [M+H]⁺: 590.3.

Step 4. A MW vessel was charged with 7 N ammonia solution in MeOH (1 mL, 7 mmol) and ethyl 2-[2-[(3,6-difluoro-2-pyridyl)amino]-3-pyridyl]-5-fluoro-6-(5-methyl-1-tetrahydropyran-2-yl-indazol-4-yl)pyrimidine-4-carboxylate (100 mg, 169.6 μmol). The vessel was sealed and heated 80° C. for 1 h then concentrated. The residue was dissolved in 4 M dioxane solution of HCl (1 mL, 4 mmol) and MeOH (1 mL) and the solution was stirred at 50° C. for 30 min in a sealed microwave vial. The volatiles were evaporated to dryness and residue was taken in 1 mL of DCM. A few drops of Et₃N were added until pH was slightly basic and the mixture was evaporated to dryness again. The solid was purified by preparative HPLC C18 column eluting with ACN/water/0.1% formic acid to provide 2-[2-[(3,6-difluoro-2-pyridyl)amino]-3-pyridyl]-5-fluoro-6-(5-methyl-1H-indazol-4-yl)pyrimidine-4-carboxamide (15 mg, 18% yield). MS: [M+H]⁺: 477.3. ¹H NMR (400 MHz, DMSO-d6) δ 13.22 (s, 1H), 11.02 (s, 1H), 8.86 (d, J=7.7 Hz, 1H), 8.54 (s, 1H), 8.35 (d, J=4.3 Hz, 1H), 8.18 (s, 1H), 7.86 (s, 1H), 7.79 (td, J=8.9, 6.4 Hz, 1H), 7.65 (d, J=8.5 Hz, 1H), 7.38 (d, J=8.6 Hz, 1H), 7.13 (dd, J=7.9, 4.6 Hz, 1H), 6.80 (dt, J=8.5, 2.8 Hz, 1H), 2.34 (s, 3H).

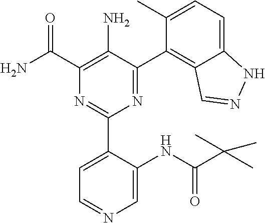 [SMILES: Cc1ccc2[nH]ncc2c1-c1nc(-c2ccncc2NC(=O)C(C)(C)C)nc(C(N)=O)c1N]

### Compound 507 (5-amino-6-(5-methyl-1H-indazol-4-yl)-3′-pivalamido-[2,4′-bipyridine]-4-carboxamide)

A solution of Intermediate M (150 mg, 497.1 μmol) in DMF (5 mL) was bubbled through with N₂ gas. Copper(I) iodide (10 mg, 52.5 μmol), LiCl (45 mg, 1.06 mmol) and Pd(dppf)Cl₂·DCM (40 mg, 50.9 μmol) were added, followed by 2,2-dimethyl-N-(4-tributylstannyl-3-pyridyl)propanamide (400 mg, 856.0 μmol). The reaction mixture was bubbled through with N₂ gas again then stirred at 120° C. for 4 h. Water was added dropwise to the cooled reaction mixture and the brown solid that was recovered by filtration was purified by preparative HPLC C18 column eluting with ACN/water/0.1% formic acid to provide 3-amino-6-[3-(2,2-dimethylpropanoylamino)-4-pyridyl]-2-(5-methyl-1H-indazol-4-yl)pyridine-4-carboxamide (20 mg, 9% yield). MS: [M+H]⁺: 445.3; ¹H NMR (400 MHz, DMSO-d6) δ 13.15 (s, 1H), 12.78 (s, 1H), 9.74 (s, 1H), 8.63 (d, J=7.2 Hz, 4H), 7.92 (s, 1H), 7.73-7.50 (m, 2H), 7.35 (d, J=8.6 Hz, 1H), 7.20-6.66 (m, 2H), 2.17 (s, 3H), 0.48 (s, 9H).

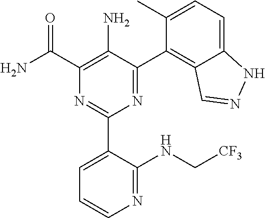 [SMILES: Cc1ccc2[nH]ncc2c1-c1nc(-c2cccnc2NCC(F)(F)F)nc(C(N)=O)c1N]

### Compound 518 (5-amino-6-(5-methyl-1H-indazol-4-yl)-2-(2-((2,2,2-trifluoroethyl)amino)pyridin-3-yl)pyrimidine-4-carboxamide)

Step 1. To a MW vial charged with Intermediate J (218 mg, 720.1 μmol), Pd(dppf)Cl₂·DCM (59 mg, 72.3 μmol), CuI (17 mg, 89.3 μmol), LiCl (58 mg, 1.37 mmol) and DMF (5 mL). The solution was bubbled through with N₂, tributyl-(2-fluoro-3-pyridyl)stannane (330 mg, 854.6 μmol) was added, the mixture was bubbled through with N₂ again, capped and transferred to a preheated (120° C.) heat block for 4.5 h. The cooled reaction mixture was added dropwise to water (20 mL). The solids were collected by filtration and washed with water. The filtrate was extracted with EtOAc (3×). The combined organic extracts was washed with brine, dried over Na₂SO₄, filtered and concentrated. The residue was combined with the filtered solids and purified by silica gel chromatography eluting with a gradient of EtOAc (10 to 100%) in heptane to provide 5-amino-2-(2-fluoro-3-pyridyl)-6-(5-methyl-1H-indazol-4-yl)pyrimidine-4-carboxamide (137 mg, 52% yield). MS: [M+H]⁺: 364.3; Step 2. To a solution of 5-amino-2-(2-fluoro-3-pyridyl)-6-(5-methyl-1H-indazol-4-yl)pyrimidine-4-carboxamide (43 mg, 118.3 μmol) in NMP (0.5 mL) was added 2,2,2-trifluoroethanamine (100 μL, 1.26 mmol). The mixture was stirred at 150° C. for 24 h. The crude reaction mixture was filtered and the filtrate was purified by preparative HPLC C18 column eluting with ACN/water/0.1% formic acid to provide 5-amino-6-(5-methyl-1H-indazol-4-yl)-2-[2-(2,2,2-trifluoroethylamino)-3-pyridyl]pyrimidine-4-carboxamide (6.24 mg, 12% yield). MS: [M+H]⁺: 443.2; ¹H NMR (400 MHz, DMSO-d6) δ 13.17 (s, 1H), 9.25 (t, J=6.4 Hz, 1H), 8.89 (dd, J=7.7, 1.9 Hz, 1H), 8.64-8.49 (m, 1H), 8.14 (dd, J=4.7, 1.9 Hz, 1H), 7.97 (d, J=2.0 Hz, 1H), 7.70 (s, 1H), 7.61 (d, J=8.6 Hz, 1H), 7.39 (d, J=8.6 Hz, 1H), 6.80 (dd, J=7.7, 4.7 Hz, 1H), 6.58 (br s, 2H), 4.37 (dtd, J=16.5, 10.0, 6.6 Hz, 1H), 4.24 (ddt, J=15.4, 10.3, 4.5 Hz, 1H), 2.26 (s, 3H).

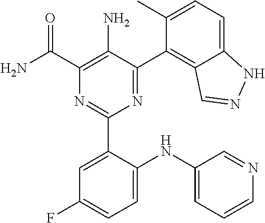 [SMILES: Cc1ccc2[nH]ncc2c1-c1nc(-c2cc(F)ccc2Nc2cccnc2)nc(C(N)=O)c1N]

### Compound 519 (5-amino-2-(5-fluoro-2-(pyridin-3-ylamino)phenyl)-6-(5-methyl-1H-indazol-4-yl)pyrimidine-4-carboxamide)

Step 1. To a solution of 2-bromo-4-fluoro-1-iodo-benzene (1.0 g, 3.32 mmol) in DMF (15 mL) were added pyridin-3-amine (350 mg, 3.72 mmol), Xantphos (300 mg, 518.5 μmol), Cs₂CO₃ (1.62 g, 4.99 mmol) and Pd(OAc)₂ (75 mg, 334.1 μmol). The mixture was degassed in vacuo, back-filled with N₂ and stirred at 110° C. for 0.5 h. Then the mixture was diluted with water and extracted with EtOAc (3×15 mL). The combined organic extracts was washed with water and brine consecutively, dried over Na₂SO₄, filtered and concentrated. The residue was purified by silica gel chromatography eluting with a gradient of EtOAc (50 to 100%) in heptane to provide N-(2-bromo-4-fluoro-phenyl)pyridin-3-amine (620 mg, 70% yield). MS: [M+H]⁺: 267.0/269.0.

Step 2. To a solution of N-(2-bromo-4-fluoro-phenyl)pyridin-3-amine (251 mg, 939.7 μmol) in dioxane (6 mL) were added bis(pinacolato)diboron (477 mg, 1.88 mmol), KOAc (326 mg, 3.32 mmol) and Pd(dppf)Cl₂ (62 mg, 84.7 μmol). The mixture was degassed in vacuo, back-filled with N₂ and stirred 80° C. for 18 h under N₂. The cooled reaction mixture was diluted with water and extracted with EtOAc (3×15 mL). The combined organic extracts were washed with water and brine consecutively, dried over Na₂SO₄, filtered and concentrated. The residue was purified by silica gel chromatography eluting with a gradient of EtOAc (50 to 100%) in heptane to provide N-[4-fluoro-2-(4,4,5,5-tetramethyl-1,3,2-dioxaborolan-2-yl)phenyl]pyridin-3-amine (220 mg, 75% yield). MS: [M+H]⁺: 315.2.

Step 3. To a solution of Intermediate J (50 mg, 165.17 μmol) in dioxane (2 mL) were added N-[4-fluoro-2-(4,4,5,5-tetramethyl-1,3,2-dioxaborolan-2-yl)phenyl]pyridin-3-amine (78 mg, 247.6 μmol), 2 M aqueous solution of K₂CO₃ (300 μL, 0.6 mmol) and Pd(dppf)Cl₂ (10.00 mg, 15.34 μmol). The mixture was degassed in vacuo, back-filled with N₂ and stirred at 110° C. for 18 h. The cooled reaction mixture was diluted with water and extracted with EtOAc (3×20 mL). The combined organic extracts was washed with water and brine consecutively, dried over Na₂SO₄, filtered and concentrated. The residue was purified by preparative HPLC C18 column eluting with ACN/water/0.1% formic acid to provide 5-amino-2-[5-fluoro-2-(3-pyridylamino)phenyl]-6-(5-methyl-1H-indazol-4-yl)pyrimidine-4-carboxamide (22 mg, 29% yield). MS: [M+H]⁺: 455.3; ¹H NMR (400 MHz, DMSO-d6) δ 13.18 (s, 1H), 10.25 (s, 1H), 8.69 (s, 1H), 8.33 (dd, J=10.7, 3.2 Hz, 1H), 7.97 (d, J=4.6 Hz, 1H), 7.91 (s, 1H), 7.85 (s, 1H), 7.69 (s, 1H), 7.60 (d, J=8.6 Hz, 1H), 7.36 (d, J=8.6 Hz, 1H), 7.33-7.24 (m, 2H), 7.13 (m, 2H), 6.64 (s, 2H), 2.20 (s, 3H).

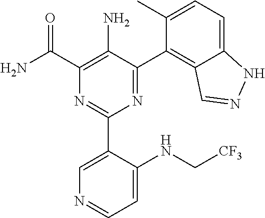 [SMILES: Cc1ccc2[nH]ncc2c1-c1nc(-c2cnccc2NCC(F)(F)F)nc(C(N)=O)c1N]

### Compound 520 (5-amino-6-(5-methyl-1H-indazol-4-yl)-2-(4-((2,2,2-trifluoroethyl)amino)pyridin-3-yl)pyrimidine-4-carboxamide)

Step 1. To a MW vial charged with Intermediate J (509 mg, 1.68 mmol), Pd(dppf)Cl₂·DCM (140 mg, 171.4 μmol), CuI (32 mg, 168.1 μmol) and LiCl (155 mg, 3.66 mmol) was added DMF (10 mL). The solution was bubbled through with N₂, tributyl-(4-fluoro-3-pyridyl)stannane (836 mg, 2.17 mmol) was added, the mixture was bubbled through with N₂ again, capped and transferred to a preheated (120° C.) heat block and stirred 3 h. The cooled reaction mixture was filtered over a Celite™ plug and washed with EtOAc. Water was added and the mixture was filtered again. The layers were separated and the aqueous layer was extracted twice with EtOAc. The combine organic extracts was washed with brine, dried over Na₂SO₄, filtered and concentrated. The residue was was purified by silica gel chromatography eluting with a gradient of MeOH (0 to 20%) in EtOAc to provide 5-amino-2-(4-fluoro-3-pyridyl)-6-(5-methyl-1H-indazol-4-yl)pyrimidine-4-carboxamide (229 mg, 37% yield). MS: [M+H]⁺: 364.3.

Step 2. To a solution of 5-amino-2-(4-fluoro-3-pyridyl)-6-(5-methyl-1H-indazol-4-yl)pyrimidine-4-carboxamide (51 mg, 140.4 μmol) in NMP (0.5 mL) was added 2,2,2-trifluoroethanamine (110 μL, 1.39 mmol). The mixture was stirred at 150° C. for 18 h. The crude mixture was purified by preparative HPLC C18 column eluting with ACN/water/0.1% formic acid and then repurified by silica gel chromatography eluting with a gradient of MeOH (1 to 20%) in DCM to provide 5-amino-6-(5-methyl-1H-indazol-4-yl)-2-[4-(2,2,2-trifluoroethylamino)-3-pyridyl]pyrimidine-4-carboxamide (8.7 mg, 14% yield). MS: [M+H]⁺: 443.2; ¹H NMR (400 MHz, DMSO-d6) δ 13.17 (s, 1H), 9.47 (s, 1H), 9.27 (t, J=6.8 Hz, 1H), 8.56 (s, 1H), 8.19 (d, J=5.9 Hz, 1H), 7.94 (s, 1H), 7.70 (s, 1H), 7.61 (d, J=8.6 Hz, 1H), 7.39 (d, J=8.6 Hz, 1H), 6.95 (d, J=6.0 Hz, 1H), 6.56 (br s, 2H), 4.25-4.01 (m, 2H), 2.25 (s, 3H).

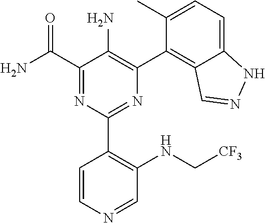 [SMILES: Cc1ccc2[nH]ncc2c1-c1nc(-c2ccncc2NCC(F)(F)F)nc(C(N)=O)c1N]

### Compound 536 (5-amino-6-(5-methyl-1H-indazol-4-yl)-2-(3-((2,2,2-trifluoroethyl)amino)pyridin-4-yl)pyrimidine-4-carboxamide)

Step 1. To a solution of N-(4-bromo-3-pyridyl)-2,2,2-trifluoro-acetamide (2.05 g, 7.62 mmol) in THF (20 mL) at 0° C. was added LiAlH₄ (293 mg, 7.72 mmol) portionwise. The mixture was stirred at 0° C. for 2.5 h. The mixture was diluted with DCM and treated with saturated aqueous Na₂SO₄ solution (2 mL), stirred for 15 min and filtered. The filtrate was concentrated and the residue was purified by silica gel chromatography eluting with a gradient of EtOAc (0 to 100%) in DCM to provide 4-bromo-N-(2,2,2-trifluoroethyl)pyridin-3-amine (310 mg, 1.22 mmol, 16% yield). MS: [M+H]⁺: 255.0/257.0.

Step 2. A MW vial was charged with Intermediate J (256 mg, 845.7 μmol), bis(pinacolato)diboron (249 mg, 980.6 μmol) and KOAc (246 mg, 2.51 mmol). Dioxane (10 mL) was added, the suspension was bubbled through with N₂, Pd(dppf)Cl₂·DCM (137 mg, 167.69 μmol) was added, the vial was capped and then transferred to a preheated (120° C.) heat block for 2 h. The cooled reaction mixture was bubbled through with N₂ then charged with 4-bromo-N-(2,2,2-trifluoroethyl)pyridin-3-amine (210 mg, 823.42 μmol) followed by dioxane (2 mL) and 2 M aqueous solution of K₂CO₃ (1.30 mL, 2.6 mmol). The vial was capped and returned to heat block (120° C.) for 1 h. The reaction mixture was cooled to rt, poured into water and extracted with DCM (3×). The combined organic layers was washed with brine, dried over Na₂SO₄, filtered and concentrated. The crude mixture was purified by preparative HPLC C18 column eluting with ACN/water/0.1% formic acid to and then repurified by silica gel chromatography eluting with a gradient of EtOAc (20 to 100%) in heptane and then with a gradient of MeOH (0 to 10%) in EtOAc to provide 5-amino-6-(5-methyl-1H-indazol-4-yl)-2-[3-(2,2,2-trifluoroethylamino)-4-pyridyl]pyrimidine-4-carboxamide (1.75 mg, 1% yield). MS: [M+H]⁺: 443.2; ¹H NMR (400 MHz, DMSO-d6) δ 13.17 (s, 1H), 8.63 (t, J=6.9 Hz, 1H), 8.59-8.52 (m, 1H), 8.43 (d, J=5.1 Hz, 1H), 8.37 (s, 1H), 7.99 (s, 2H), 7.70 (s, 1H), 7.62 (d, J=8.6 Hz, 1H), 7.39 (d, J=8.6 Hz, 1H), 6.70 (br s, 2H), 4.14 (tt, J=15.6, 9.0 Hz, 2H), 2.25 (s, 3H).

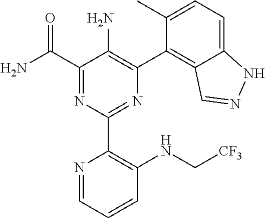 [SMILES: Cc1ccc2[nH]ncc2c1-c1nc(-c2ncccc2NCC(F)(F)F)nc(C(N)=O)c1N]

### Compound 536 (5-amino-6-(5-methyl-1H-indazol-4-yl)-2-(3-((2,2,2-trifluoroethyl)amino)pyridin-2-yl)pyrimidine-4-carboxamide)

Step 1. To a solution of 3-amino-2-bromo-4-picoline (1.0 g, 5.78 mmol) in dry DCM (8 mL) was added trifluroacetic anhydride (0.804 mL, 5.78 mmol). The reaction mixture was stirred at rt for 30 minutes. Water was added and the aqueous layer was extracted with DCM (3×). The combined organic layers was washed with brine, dried over MgSO₄, filtered and concentrated to dryness to afford 1.47 g of N-(2-bromopyridin-3-yl)-2,2,2-trifluoroacetamide which was used as such in the subsequent step without further purification.

Step 2. To a solution of N-(2-bromopyridin-3-yl)-2,2,2-trifluoroacetamide (1.47 g, 5.19 mmol) in dry THF (52 mL) at 0° C. under nitrogen atmosphere was added LiAlH₄ (2 M in THF, 2.36 mL, 4.72 mmol). The reaction mixture was stirred at 0° C. for 2 h. The mixture was diluted with EtOAc followed by addition of saturated aqueous Na₂SO₄ solution (300 μL). The mixture was filtered through a pad of Celite™ and the filtrate was concentrated. The residue was purified by silica gel chromatography eluting with a gradient of acetonitrile (0 to 100%) in DCM to provide 2-bromo-N-(2,2,2-trifluoroethyl)pyridine-3-amine. MS: [M+H]⁺: 254.9/256.9.

Step 3. Intermediate J (100 mg, 0.33 mmol), bis(pinacolato)diboron (125 mg, 0.496 mmol), PdCl₂(dppf) (27 mg, 0.033 mmol) and KOAc (98 mg, 0.991 mmol) were charged in a microwave vial. The vial was capped and DMF (1.2 mL) was added under nitrogen. The reaction mixture was then degassed by bubbling nitrogen for 10 minutes in an ultrasonic bath. The reaction mixture was stirred at 120° C. for 1 h. The cooled reaction mixture was diluted with EtOAc and filtered on Celite™ and the filtrate was concentrated. To this residue was added 2-bromo-N-(2,2,2-trifluoroethyl)pyridine-3-amine (26 mg, 0.099 mmol), Pd₂(dba)₃ (30 mg, 0.033 mmol), tri-tert-butylphosphonium tetrafluoroborate (19 mg, 0.661 mmol), dioxane (1.1 mL) and 2 M aqueous solution of K₃PO₄ (330 μL, 0.660 mmol). The reaction mixture was then degassed by bubbling nitrogen for 10 minutes in an ultrasonic bath and heated to 100° C. for 1 h. The crude mixture was purified by preparative HPLC C18 column eluting with ACN/water/0.1% formic acid to provide 5-amino-6-(5-methyl-1H-indazol-4-yl)-2-(3-((2,2,2-trifluoroethyl)amino) yridine-2-yl)pyrimidine-4-carboxamide (3 mg, 2%). MS: [M+H]⁺: 442.9; ¹H NMR (400 MHz, DMSO-d6) δ 1H-NMR (400 MHz, DMSO): δ 13.14 (s; 1H); 8.18 (s; 1H); 8.11 (s; 1H); 8.06 (t; J=6.89 Hz; 1H); 7.98-8.02 (m; 2H); 7.67 (s; 1H); 7.59 (d; J=8.59 Hz; 1H); 7.38 (t; J=9.70 Hz; 2H); 7.24 (t; J=5.86 Hz; 1H); 6.56 (s; 2H); 4.06 (t; J=9.46 Hz; 2H); 2.23 (s; 3H).

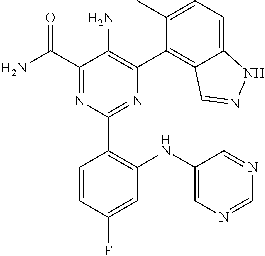 [SMILES: Cc1ccc2[nH]ncc2c1-c1nc(-c2ccc(F)cc2Nc2cncnc2)nc(C(N)=O)c1N]

### Compound 541 (5-amino-2-(4-fluoro-2-(pyrimidin-5-ylamino)phenyl)-6-(5-methyl-1H-indazol-4-yl)pyrimidine-4-carboxamide)

Step 1. To a solution of 1-bromo-4-fluoro-2-iodo-benzene (150 mg, 498.5 μmol) in DMF (3 mL) were added pyrimidin-5-amine (52 mg, 546.8 μmol), cesium carbonate (245 mg, 752.0 μmol), Xantphos (44 mg, 76.0 μmol) and Pd(OAc)₂ (11 mg, 49.0 μmol). The mixture was degassed in vacuo, back-filled with N₂ and stirred at 110° C. for 2 h. The volatiles were removed in vacuo and the residue was purified by silica gel chromatography eluting with a gradient of EtOAc (20 to 100%) in heptane to provide N-(2-bromo-5-fluoro-phenyl)pyrimidin-5-amine (110 mg, 82% yield). MS: [M+H]⁺: 268.1.

Step 2. To a solution of N-(2-bromo-5-fluoro-phenyl)pyrimidin-5-amine (20 mg, 74.6 μmol) in DMF (1 mL) were added Intermediate X (45 mg, 80.7 μmol), CuI (3 mg, 15.8 μmol) and Pd(dtbpf)Cl₂ (5 mg, 7.7 μmol). The mixture was degassed in vacuo, back-filled with N₂ and stirred at 120° C. for 5 h in a microwave reactor. The crude reaction mixture was filtered and the filtrate was purified by preparative HPLC C18 column eluting with ACN/water/0.1% formic acid to provide 5-amino-2-[4-fluoro-2-(pyrimidin-5-ylamino)phenyl]-6-(5-methyl-1H-indazol-4-yl)pyrimidine-4-carboxamide (10 mg, 29% yield). MS: [M+H]⁺: 456.2; ¹H NMR (400 MHz, DMSO-d6) δ 13.16 (s, 1H), 10.81 (s, 1H), 8.66 (s, 1H), 8.59 (s, 1H), 8.57-8.52 (m, 1H), 8.29 (s, 2H), 7.91 (s, 1H), 7.69 (s, 1H), 7.59 (d, J=8.7 Hz, 1H), 7.34 (d, J=8.6 Hz, 1H), 7.08 (dd, J=11.4, 2.6 Hz, 1H), 6.82-6.72 (m, 1H), 6.57 (s, 2H), 2.21 (s, 3H).

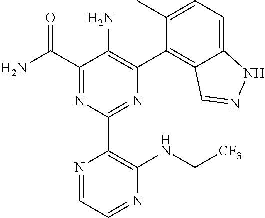 [SMILES: Cc1ccc2[nH]ncc2c1-c1nc(-c2nccnc2NCC(F)(F)F)nc(C(N)=O)c1N]

### Compound 555 (5-amino-2-(4-fluoro-2-(pyrimidin-5-ylamino)phenyl)-6-(5-methyl-1H-indazol-4-yl)pyrimidine-4-carboxamide)

Step 1. To a solution of 3-bromopyrazin-2-amine (0.5 g, 2.87 mmol) in dry DCM (6.0 mL) was added trifluoroacetic anhydride (0.6 mL, 2.87 mmol). The reaction mixture was stirred at rt for 30 min. Water was added and the aqueous layer was extracted with DCM (3×). The combined organic layers was washed with brine, dried over MgSO₄, filtered and concentrated to dryness to afford 540 mg of N-(3-bromopyrazin-2-yl)-2,2,2-trifluoroacetamide which was used as such in the subsequent step without further purification.

Step 2. To a solution of N-(3-bromopyrazin-2-yl)-2,2,2-trifluoroacetamide (0.54 g, 1.91 mmol) in dry THF (19 mL) at 0° C. under nitrogen atmosphere was added LiAlH₄ (2 M in THF, 0.87 mL, 1.73 mmol). The reaction mixture was stirred at 0° C. for 2 h. The mixture was diluted with EtOAc followed by addition of saturated aqueous Na₂SO₄ solution (300 μL). The mixture was filtered through a pad of Celite™ and the filtrate was concentrated. The residue was purified by silica gel chromatography eluting with a gradient of acetonitrile (0 to 100%) in DCM to provide 3-bromo-N-(2,2,2-trifluoroethyl)pyrazin-2-amine. MS: [M+H]⁺: 255.8/257.8.

Step 3. Intermediate J (100 mg, 0.33 mmol), bis(pinacolato)diboron (126 mg, 0.496 mmol), PdCl2(dppf) (27 mg, 0.033 mmol) and KOAc (98 mg, 0.991 mmol) were charged in a microwave vial. The vial was capped and DMF (1.2 mL) was added under nitrogen. The reaction mixture was then degassed by bubbling nitrogen for 10 minutes in an ultrasonic bath. The reaction mixture was stirred to 120° C. for 1 h. Upon cooling to rt, the reaction mixture was diluted with EtOAc, filtered on Celite™ and the filtrate was concentrated. To this residue was added 3-bromo-N-(2,2,2-trifluoroethyl)pyrazin-2-amine (26 mg, 0.099 mmol), Pd₂(dba)₃ (30 mg, 0.033 mmol), tri-tert-butylphosphonium tetrafluoroborate (19 mg, 0.066 mmol), dioxane (1.1 mL) and 2M aqueous solution of K₃PO₄ (330 μL, 0.660 mmol). The reaction mixture was then degassed by bubbling nitrogen for 10 minutes in an ultrasonic bath and stirred at 100° C. for 1 h. The crude mixture was purified by preparative HPLC C18 column eluting with ACN/water/0.1% TFA to provide 5-amino-6-(5-methyl-1H-indazol-4-yl)-2-(3-((2,2,2-trifluoroethyl)amino)pyrazin-2-yl)pyrimidine-4-carboxamide (6 mg, 4% yield). MS: [M+H]⁺: 444.1; ¹H NMR (400 MHz, DMSO-d6) δ 8.91 (t; J=6.42 Hz; 1H); 8.18 (d; J=2.79 Hz; 2H); 8.04 (d; J=2.55 Hz; 1H); 8.02 (s; 1H); 7.70 (s; 1H); 7.61 (d; J=8.56 Hz; 1H); 7.37 (d; J=8.58 Hz; 1H); 4.26-4.35 (m; 2H); 2.24 (s; 3H).

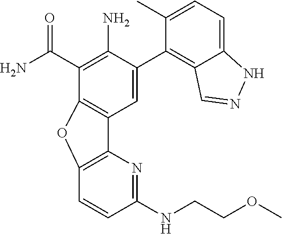 [SMILES: COCCNc1ccc2oc3c(C(N)=O)c(N)c(-c4c(C)ccc5[nH]ncc45)cc3c2n1]

### Compound 558 (7-amino-2-((2-methoxyethyl)amino)-8-(5-methyl-1H-indazol-4-yl)benzofuro[3,2-b]pyridine-6-carboxamide)

Step 1. A microwave tube was loaded with Intermediate Z (70.0 mg, 0.196 mmol) and 2-methoxyethan-1-amine (1 mL, 11.5 mmol). The reaction mixture was heated to 160° C. for 18 h. Upon cooling to rt, the volatiles were removed in vacuo. The residue was purified by silica gel chromatography eluting with a gradient of EtOAc (30 to 100%) in hexanes to provide 7-amino-2-((2-methoxyethyl)amino)-8-(5-methyl-1H-indazol-4-yl)benzofuro[3,2-b]pyridine-6-carbonitrile (31 mg, 38% yield). MS: [M+H]⁺: 413.1.

Step 2. To a suspension of 7-amino-2-((2-methoxyethyl)amino)-8-(5-methyl-1H-indazol-4-yl)benzofuro[3,2-b]pyridine-6-carbonitrile (31 mg, 0.075 mmol) in 4:1 EtOH-water (5 mL) was added hydrido(dimethylphosphinous acid-kP)[hydrogen bis(dimethylphosphinito-kP)]platinum (II) (Ghaffar-Parkins Catalyst) (9 mg, 0.021 mmol). The mixture was heated to 80° C. for 30 h. Upon cooling to rt, the volatiles were removed in vacuo and the residue was purified by silica gel chromatography eluting with a gradient of EtOAc (30 to 100%) in hexanes to provide 7-amino-2-((2-methoxyethyl)amino)-8-(5-methyl-1H-indazol-4-yl)benzofuro[3,2-b]pyridine-6-carboxamide carboxamide (16 mg, 50% yield). MS: [M+H]⁺: 431.1; ¹H NMR (400 MHz, DMSO-d6) δ 13.06 (s; 1H); 7.81 (s; 2H); 7.72 (d; J=8.97 Hz; 1H); 7.50-7.51 (m; 3H); 7.35 (d; J=8.60 Hz; 1H); 6.48-6.54 (m; 4H); 3.44-3.46 (m; 4H); 3.24 (s; 3H); 2.17 (s; 3H).

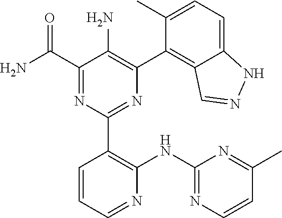 [SMILES: Cc1ccnc(Nc2ncccc2-c2nc(C(N)=O)c(N)c(-c3c(C)ccc4[nH]ncc34)n2)n1]

### Compound 565 (5-amino-6-(5-methyl-1H-indazol-4-yl)-2-(2-((4-methylpyrimidin-2-yl)amino)pyridin-3-yl)pyrimidine-4-carboxamide)

Step 1. To a solution of Intermediate AB (60 mg, 126.7 μmol) in toluene (1.5 mL) were added 2-chloro-4-methyl-pyrimidine (32 mg, 248.9 μmol), Cs₂CO₃ (120 mg, 368.3 μmol), Xantphos (18 mg, 31.1 μmol) and Pd(OAc)₂ (3.6 mg, 16.0 μmol). The mixture was degassed in vacuo, back-filled with N₂ and stirred at 110° C. for 2 h. The volatiles were removed in vacuo and the residue was purified by silica gel chromatography eluting with a gradient of EtOAc (20 to 100%) in heptane to provide ethyl 5-amino-2-[2-[(4-methylpyrimidin-2-yl)amino]-3-pyridyl]-6-(5-methyl-1-tetrahydropyran-2-yl-indazol-4-yl)pyrimidine-4-carboxylate (55 mg, 77% yield). MS: [M+H]⁺: 566.5.

Step 2. A MW vial was charged with ethyl 5-amino-2-[2-[(4-methylpyrimidin-2-yl)amino]-3-pyridyl]-6-(5-methyl-1-tetrahydropyran-2-yl-indazol-4-yl)pyrimidine-4-carboxylate (55 mg, 97.2 μmol). 7 N Ammonia solution in MeOH (3.5 mL, 24.5 mmol) was added, the vessel was sealed and stirred at 85° C. for 3 h. The solvents were removed under reduced pressure. The solid was suspended in DCM (1 mL) and then treated with 4 M HCl solution in dioxane (380 μL, 1.52 mmol). The mixture was stirred at rt for 2 h. The volatiles were removed in vacuo and the residue was purified by preparative HPLC C18 column eluting with ACN/water/0.1% formic acid to provide 5-amino-6-(5-methyl-1H-indazol-4-yl)-2-[2-[(4-methylpyrimidin-2-yl)amino]-3-pyridyl]pyrimidine-4-carboxamide (5 mg, 20% yield). MS: [M+H]⁺: 453.5; ¹H NMR (400 MHz, DMSO-d6) δ 13.12 (s, 1H), 8.92 (s, 1H), 8.49 (d, J=6.1 Hz, 1H), 8.35 (s, 1H), 8.27 (d, J=5.1 Hz, 1H), 8.03 (s, 1H), 7.68-7.55 (m, 2H), 7.36 (d, J=8.6 Hz, 1H), 7.17 (s, 1H), 6.82 (s, 1H), 6.68 (s, 2H), 2.22 (s, 3H), 2.20 (s, 3H).

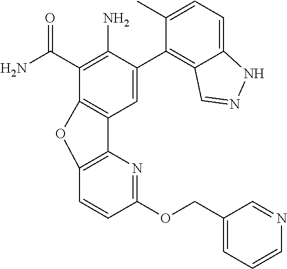 [SMILES: Cc1ccc2[nH]ncc2c1-c1cc2c(oc3ccc(OCc4cccnc4)nc32)c(C(N)=O)c1N]

### Compound 569 (7-amino-8-(5-methyl-1H-indazol-4-yl)-2-(pyridin-3-ylmethoxy)benzofuro[3,2-b]pyridine-6-carboxamide)

Step 1. To a solution of pyridine-3-ylmethanol (92 mg, 0.843 mmol) in THF (1 mL) at rt was added NaH (60% dispersion in mineral oil, 45 mg, 1.125 mmol). The mixture was stirred at rt for 15 min and this reaction mixture was added to a solution of Intermediate Z (60 mg, 0.168 mmol) in THF (2 mL). The mixture was heated to 60° C. for 4 h. Upon cooling to rt, the reaction mixture was diluted with water and EtOAc. The layers were partitioned, the aqueous layer was extracted with EtOAc. The combined organic layers was washed with brine, dried over MgSO₄, filtered and concentrated. The residue was purified by silica gel chromatography eluting with a gradient of EtOAc (30 to 100%) in hexanes to provide 7-amino-8-(5-methyl-1H-indazol-4-yl)-2-(yridine-3-ylmethoxy)benzofuro[3,2-b]pyridine-6-carbonitrile (40 mg, 51% yield). MS: [M+H]⁺: 447.1.

Step 2. To a suspension of 7-amino-8-(5-methyl-1H-indazol-4-yl)-2-(pyridine-3-ylmethoxy)benzofuro[3,2-b]pyridine-6-carbonitrile (45 mg, 0.101 mmol) in 4:1 EtOH-water (5 mL) was added hydrido(dimethylphosphinous acid-kP)[hydrogen bis(dimethylphosphinito-kP)]platinum (II) (Ghaffar-Parkins Catalyst) (9 mg, 0.021 mmol). The mixture was heated to 90° C. for 18 h. Upon cooling to rt, the volatiles were removed in vacuo and the residue was purified by silica gel chromatography eluting with a gradient of EtOAc (30 to 100%) in hexanes to provide of 7-amino-8-(5-methyl-1H-indazol-4-yl)-2-(pyridine-3-ylmethoxy)benzofuro[3,2-b]pyridine-6-carboxamide (22 mg, 47% yield). MS: [M+H]⁺: 465.1; ¹H NMR (400 MHz, DMSO-d6) δ 13.08 (s; 1H); 8.70 (s; 1H); 8.48 (d; J=4.77 Hz; 1H); 8.04 (d; J=8.85 Hz; 1H); 7.88-7.90 (m; 3H); 7.66 (s; 1H); 7.52-7.54 (m; 2H); 7.35-7.37 (m; 2H); 6.82 (d; J=8.85 Hz; 1H); 6.66 (s; 2H); 5.44 (s; 2H); 2.18 (s; 3H).

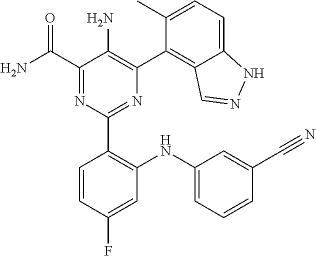 [SMILES: Cc1ccc2[nH]ncc2c1-c1nc(-c2ccc(F)cc2Nc2cccc(C#N)c2)nc(C(N)=O)c1N]

### Compound 580 (5-amino-2-(2-((3-cyanophenyl)amino)-4-fluorophenyl)-6-(5-methyl-1H-indazol-4-yl)pyrimidine-4-carboxamide)

Step 1. To a solution of Intermediate AB (13 g, 31.3 mmol), 5-fluoro-2-(4,4,5,5-tetramethyl-1,3,2-dioxaborolan-2-yl)aniline (11.85 g, 50.0 mmol), Pd(dppf)Cl₂·DCM (2.8 g, 3.43 mmol) in dioxane (300 mL) was added 2M aqueous solution of K₂CO₃ (47 mL, 94 mmol). The reaction mixture was stirred at 100° C. for 5 h. The reaction mixture was cooled to rt, filtered through Celite™, and the filtrate was concentrated. The residue was purified by silica gel chromatography eluting with a gradient of EtOAc (0 to 100%) in heptane to provide ethyl 5-amino-2-(2-amino-4-fluoro-phenyl)-6-(5-methyl-1-tetrahydropyran-2-yl-indazol-4-yl)pyrimidine-4-carboxylate (13.2 g, 86% yield). MS: [M+H]⁺: 491.2.

Step 2. To a MW vial charged with ethyl 5-amino-2-(2-amino-4-fluoro-phenyl)-6-(5-methyl-1-tetrahydropyran-2-yl-indazol-4-yl)pyrimidine-4-carboxylate (49 mg, 100.0 μmol), 3-iodobenzonitrile (29 mg, 126.6 μmol), CS2CO₃ (56 mg, 171.9 μmol), Pd(OAc)₂ (4 mg, 17.8 μmol) and Xantphos (18 mg, 31.1 μmol) was added dioxane (3 mL). Nitrogen was bubbled through the mixture, the vial was capped and transferred to a preheated (110° C.) heat block for 80 min. The cooled reaction mixture was adsorbed on silica and the residue was purified by silica gel chromatography eluting with a gradient of EtOAc (0 to 100%) in heptane to provide ethyl 5-amino-2-[2-(3-cyanoanilino)-4-fluoro-phenyl]-6-(5-methyl-1-tetrahydropyran-2-yl-indazol-4-yl)pyrimidine-4-carboxylate which was used as such for next step.

Step 3. A MW vial was charged with ethyl 5-amino-2-[2-(3-cyanoanilino)-4-fluoro-phenyl]-6-(5-methyl-1-tetrahydropyran-2-yl-indazol-4-yl)pyrimidine-4-carboxylate from the previous step and 7 N ammonia solution in MeOH (2 mL, 14 mmol). The vial was capped and transferred to a preheated (100° C.) heat block. After 4 h the mixture was cooled down to rt and concentrated to dryness. MeOH (2 mL) was added to the solid followed by 4 M dioxane solution of HCl (530 μL, 2.12 mmol). The mixture was stirred at 45° C. for 35 min then concentrated to dryness. The solid was dissolved in MeOH, basicified with Et₃N and concentrated again. The residue was purified by preparative HPLC C18 column eluting with ACN/water/0.1% formic acid to provide 5-amino-2-[2-(3-cyanoanilino)-4-fluoro-phenyl]-6-(5-methyl-1H-indazol-4-yl)pyrimidine-4-carboxamide (18 mg, 38% yield over 2 steps). MS: [M+H]⁺: 479.2; ¹H NMR (400 MHz, DMSO-d6) δ 13.21 (s, 1H), 10.84 (s, 1H), 8.62 (d, J=2.1 Hz, 1H), 8.58 (dd, J=8.9, 7.1 Hz, 1H), 7.95 (s, 1H), 7.71 (s, 1H), 7.67-7.58 (m, 1H), 7.42-7.34 (m, 2H), 7.30 (dt, J=7.6, 1.3 Hz, 1H), 7.23-7.17 (m, 1H), 7.15 (t, J=1.9 Hz, 1H), 7.12 (dd, J=11.6, 2.6 Hz, 1H), 6.79 (ddd, J=9.0, 7.9, 2.6 Hz, 1H), 6.59 (s, 2H), 2.24 (s, 3H).

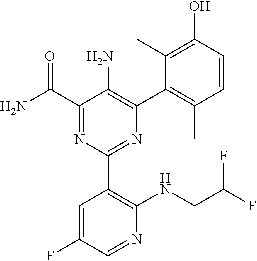 [SMILES: Cc1ccc(O)c(C)c1-c1nc(-c2cc(F)cnc2NCC(F)F)nc(C(N)=O)c1N]

### Compound 700 (5-amino-2-(2-((2,2-difluoroethyl)amino)-5-fluoropyridin-3-yl)-6-(3-hydroxy-2,6-dimethylphenyl)pyrimidine-4-carboxamide)

Step 1. A MW vial was charged with Intermediate AG (103 mg, 306.7 μmol), and Pd(dppf)Cl₂·DCM (31 mg, 38.0 μmol), Intermediate AH (85 mg, 386.4 μmol). Dioxane (2 mL) and 2 M aqueous solution of K₂CO₃ (450 μL, 0.9 mmol) were added, N2 was bubbled through the solution for 10 min, the vial was capped and transferred to a preheated (100° C.) heat block overnight. The cooled reaction mixture was diluted with water and DCM. The pH was adjusted to 5 with 1 N aqueous solution of HCl and the layers were separated. The aqueous layer was extracted with DCM (4×). The combined organic extracts was concentrated to dryness. This solid was dissolved in THF (3 mL) and MeOH (3 mL) and treated with 4 M aqueous solution of sodium hydroxide (770 μL, 3.08 mmol). The mixture was stirred for 90 min and then concentrated. Water was added to the residue and pH was adjusted to pH 4-5 with 1 N aqueous solution of HCl. The solids were collected by filtration, washed with water then dried in vacuo, affording 5-amino-2-[2-(2,2-difluoroethylamino)-5-fluoro-3-pyridyl]-6-(3-methoxy-2,6-dimethyl-phenyl)pyrimidine-4-carboxylic acid (130 mg, 95% yield). MS: [M+H]⁺: 448.3.

Step 2. To a vial containing 5-amino-2-[2-(2,2-difluoroethylamino)-5-fluoro-3-pyridyl]-6-(3-methoxy-2,6-dimethyl-phenyl)pyrimidine-4-carboxylic acid (130 mg, 290.6 μmol), ammonium chloride (85 mg, 1.59 mmol) and HATU (141 mg, 370.8 μmol) was added DMF (1.5 mL) and DIPEA (300 μL, 1.72 mmol). The vial was capped and stirred at 60° C. for 1 h. The cooled reaction mixture was diluted with water and the solids were collected by filtration and washed with water. The residue was purified by silica gel chromatography eluting with a gradient of EtOAc (0 to 100%) in heptane to provide 5-amino-2-[2-(2,2-difluoroethylamino)-5-fluoro-3-pyridyl]-6-(3-hydroxy-2,6-dimethyl-phenyl)pyrimidine-4-carboxamide (62 mg, 49% yield). MS: [M+H]⁺: 447.3.

Step 3. To a solution of 5-amino-2-[2-(2,2-difluoroethylamino)-5-fluoro-3-pyridyl]-6-(3-methoxy-2,6-dimethyl-phenyl)pyrimidine-4-carboxamide (62 mg, 138.9 μmol) in DCM (1 mL) was added 1 M DCM solution of tribromoborane (700 μL, 0.7 mmol). The mixture was stirred for 90 min, concentrated to dryness, coevaporated with MeOH then with a mixture of MeOH and Et₃N. The residue was purified by preparative HPLC C18 column eluting with ACN/water/0.1% formic acid to provide 5-amino-2-[2-(2,2-difluoroethylamino)-5-fluoro-3-pyridyl]-6-(3-hydroxy-2,6-dimethyl-phenyl)pyrimidine-4-carboxamide (16 mg, 27% yield). MS: [M+H]⁺: 433.3. ¹H NMR (400 MHz, DMSO-d6) δ 9.39 (s, 1H), 9.02 (t, J=6.0 Hz, 1H), 8.85 (dd, J=10.3, 3.0 Hz, 1H), 8.71 (br s, 1H), 8.12 (d, J=3.0 Hz, 1H), 7.93 (br s, 1H), 7.02 (d, J=8.2 Hz, 1H), 6.85 (d, J=8.2 Hz, 1H), 6.50 (br s, 2H), 6.08 (tt, J=56.2, 3.8 Hz, 1H), 3.84 (ddt, J=15.5, 11.2, 5.2 Hz, 2H), 1.90 (s, 3H), 1.83 (s, 3H).

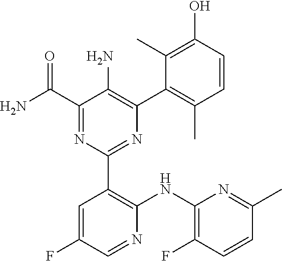 [SMILES: Cc1ccc(F)c(Nc2ncc(F)cc2-c2nc(C(N)=O)c(N)c(-c3c(C)ccc(O)c3C)n2)n1]

### Compound 715 (5-amino-2-(2-((2,2-difluoroethyl)amino)-5-fluoropyridin-3-yl)-6-(3-hydroxy-2,6-dimethylphenyl)pyrimidine-4-carboxamide)

Step 1. A RBF was loaded with Intermediate AG (199.00 mg, 592.6 μmol), Intermediate AH (208 mg, 873.7 μmol) in dioxane (4 mL) and 2 M aqueous solution of K₂CO₃ (732 μL, 1.46 mmol). The mixture was bubbled through with N2 and Pd(dtbpf)Cl₂ (45 mg, 69.1 μmol) was added. The mixture was bubbled through again with N2 and stirred at 110° C. for 10 h. The cooled reaction mixture was diluted with DCM and water and the layers were separated. The aqueous layer was extracted with DCM (4×) and concentrated. The residue was purified by silica gel chromatography eluting with a gradient of EtOAc (20 to 100%) in heptane to provide ethyl 5-amino-2-(2-amino-5-fluoro-3-pyridyl)-6-(3-methoxy-2,6-dimethyl-phenyl)pyrimidine-4-carboxylate (53 mg, 22% yield). MS: [M+H]⁺: 412.3.

Step 2. A MW vial was charged with ethyl 5-amino-2-(2-amino-5-fluoro-3-pyridyl)-6-(3-methoxy-2,6-dimethyl-phenyl)pyrimidine-4-carboxylate (53 mg, 128.8 μmol), 2-bromo-3-fluoro-6-methyl-pyridine (56 mg, 294.72 μmol), Pd(OAc)₂ (5 mg, 22.3 μmol), Xantphos (24 mg, 41.5 μmol) and cesium carbonate (138 mg, 423.6 μmol). Toluene (2 mL) was added, the mixture was bubbled through with N₂, capped and transferred to a preheated (110° C.) heat block overnight. The cooled reaction mixture was concentrated and the residue was purified by silica gel chromatography eluting with a gradient of EtOAc (0 to 100%) in heptane to provide ethyl 5-amino-2-[5-fluoro-2-[(3-fluoro-6-methyl-2-pyridyl)amino]-3-pyridyl]-6-(3-methoxy-2,6-dimethyl-phenyl)pyrimidine-4-carboxylate (23 mg, 34% yield). MS: [M+H]⁺: 412.3.

Step 3. A MW vial containing ethyl 5-amino-2-[5-fluoro-2-[(3-fluoro-6-methyl-2-pyridyl)amino]-3-pyridyl]-6-(3-methoxy-2,6-dimethyl-phenyl)pyrimidine-4-carboxylate (23 mg, 44.2 μmol) and 7 N ammonia solution in MeOH (3 mL, 21 mmol) was capped and transferred to a preheated (100° C.) heat block for 1 h. The cooled reaction mixture was concentrated to dryness then dissolved in DCM (1 mL) and treated with 1 M DCM solution of tribromoborane (270 μL 0.27 mmol). The mixture was stirred at rt for 1 h. The mixture was concentrated to dryness, coevaporated with MeOH then with MeOH/Et₃N. The residue was purified by preparative HPLC C18 column eluting with ACN/water/0.1% formic acid to provide 5-amino-2-[5-fluoro-2-[(3-fluoro-6-methyl-2-pyridyl)amino]-3-pyridyl]-6-(3-hydroxy-2,6-dimethyl-phenyl)pyrimidine-4-carboxamide (0.6 mg, 3% yield). MS: [M+H]⁺: 478.4.

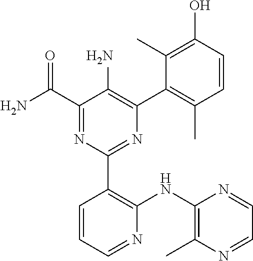 [SMILES: Cc1ccc(O)c(C)c1-c1nc(-c2cccnc2Nc2nccnc2C)nc(C(N)=O)c1N]

### Compound 727 (5-amino-6-(3-hydroxy-2,6-dimethylphenyl)-2-(2-((3-methylpyrazin-2-yl)amino)pyridin-3-yl)pyrimidine-4-carboxamide)

Step 1. A MW vial was loaded with Intermediate AG (328 mg, 976.8 μmol), 3-(4,4,5,5-tetramethyl-1,3,2-dioxaborolan-2-yl)pyridin-2-amine (322 mg, 1.46 mmol) and Pd(dtbpf)Cl₂ (74 mg, 113.5 μmol). Dioxane (5 mL) and 2 M aqueous solution of K₂CO₃ (1.20 mL, 2.4 mmol) were added, the mixture was bubbled through with N2, capped and stirred at 110° C. for 90 min. The cooled reaction mixture was diluted with DCM and water and the layers were separated. The aqueous layer was extracted with DCM (4×). The combined organic layers was concentrated and the residue was purified by silica gel chromatography eluting with a gradient of EtOAc (20 to 100%) in heptane to provide ethyl 5-amino-2-(2-amino-3-pyridyl)-6-(3-methoxy-2,6-dimethyl-phenyl)pyrimidine-4-carboxylate (365 mg, 95% yield). MS: [M+H]⁺: 394.3.

Step 2. A MW vial was charged with ethyl 5-amino-2-(2-amino-3-pyridyl)-6-(3-methoxy-2,6-dimethyl-phenyl)pyrimidine-4-carboxylate (150 mg, 381.3 μmol), 2-chloro-3-methyl-pyrazine (77 mg, 598.9 μmol), Pd(OAc)₂ (11 mg, 49.0 μmol), Xantphos (44 mg, 76.0 μmol) and cesium carbonate (310 mg, 951.5 μmol). Toluene (3 mL) was added, the mixture was bubbled through with N₂, capped and transferred to a preheated (110° C.) heat block and stirred overnight. The cooled reaction mixture was concentrated to dryness. Water was added and pH was adjusted to pH 4-5 with 1 N aqueous solution of HCl. The solids were collected by filtration and washed with water. The solid was taken in DCM/MeOH, dried over MgSO₄, filtered and concentrated. Ammonium chloride (80 mg, 1.50 mmol) and HATU (152 mg, 399.8 μmol) were added to the solid followed by DMF (1.5 mL) and DIPEA (311 μL, 1.79 mmol). The mixture was stirred at 60° C. for 75 min. Water was added to the cooled reaction mixture, the solids were collected by filtration and purified by silica gel chromatography eluting with a gradient of EtOAc (0 to 100%) in heptane to provide 61 mg of a mixture containing the desired product as a minor component. To a solution of this material in DCM (2 mL) was added 1 M DCM solution of tribromoborane (700 μL, 0.7 mmol). The solution was stirred for 2 h, concentrated to dryness, coevaporated with MeOH then with MeOH/Et₃N. The residue was purified by preparative HPLC C18 column eluting with ACN/water/0.1% formic acid to provide 5-amino-6-(3-hydroxy-2,6-dimethyl-phenyl)-2-[2-[(3-methylpyrazin-2-yl)amino]-3-pyridyl]pyrimidine-4-carboxamide (7 mg, 4% yield). MS: [M+H]⁺: 443.3; ¹H NMR (400 MHz, DMSO-d6) δ 11.53 (s, 1H), 9.43 (s, 1H), 9.09 (d, J=7.8 Hz, 1H), 8.68 (s, 1H), 8.25 (d, J=3.8 Hz, 1H), 8.15-8.10 (m, 1H), 8.06 (d, J=2.6 Hz, 1H), 7.99 (s, 1H), 7.07 (dd, J=7.9, 4.7 Hz, 1H), 7.03 (d, J=8.2 Hz, 1H), 6.88 (d, J=8.2 Hz, 1H), 6.56 (s, 2H), 1.96 (s, 3H), 1.90 (s, 3H), 1.82 (s, 3H).

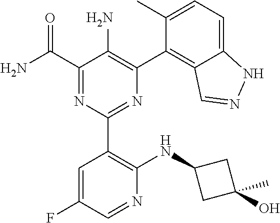 [SMILES: Cc1ccc2[nH]ncc2c1-c1nc(-c2cc(F)cnc2N[C@H]2C[C@@](C)(O)C2)nc(C(N)=O)c1N]

### Compound 733 (5-amino-2-(5-fluoro-2-(((1s,3s)-3-hydroxy-3-methylcyclobutyl)amino)pyridin-3-yl)-6-(5-methyl-1H-indazol-4-yl)pyrimidine-4-carboxamide)

Step 1. A MW vial was charged with Intermediate J (1.2 g, 3.96 mmol), Pd(dtbpf)Cl₂ (515 mg, 790.2 μmol), (2,5-difluoro-3-pyridyl)boronic acid (1.95 g, 12.28 mmol), dioxane (20 mL) and 2 M aqueous solution of potassium carbonate (5.95 mL, 11.9 mmol). N₂ was bubbled through and the mixture and the vial was capped and transferred to a preheated (100° C.) heat block and stirred for 2 h. The reaction mixture was cooled to rt, poured into water and extracted with EtOAc (3×). The combined organic layers was washed with brine, dried over Na₂SO₄, filtered and concentrated. The residue was purified by silica gel chromatography eluting with a gradient of EtOAc (0 to 100%) in heptane to provide 5-amino-2-(2,5-difluoro-3-pyridyl)-6-(5-methyl-1H-indazol-4-yl)pyrimidine-4-carboxamide (720 mg, 48% yield) which was used as such for next step. MS: [M+H]⁺: 382.3.

Step 2. To a solution of 3-amino-1-methyl-cyclobutanol hydrochloride (145 mg, 1.05 mmol) and 5-amino-2-(2,5-difluoro-3-pyridyl)-6-(5-methyl-1H-indazol-4-yl)pyrimidine-4-carboxamide (200 mg, 524.5 μmol) in NMP (1 mL) was added DIPEA (365 μL, 2.10 mmol). The reaction mixture was stirred in a sealed microwave vial at 140° C. for 6 h. The volatiles were evaporated and the crude reaction mixture was filtered and the filtrate was purified by preparative HPLC C18 column eluting with ACN/water/0.1% formic acid to provide 5-amino-2-(5-fluoro-2-(((1s,3s)-3-hydroxy-3-methylcyclobutyl)amino)pyridin-3-yl)-6-(5-methyl-1H-indazol-4-yl)pyrimidine-4-carboxamide (40 mg, 16% yield). MS: [M+H]⁺: 463.3; ¹H NMR (400 MHz, DMSO-d6) δ 13.18 (s, 1H), 8.98 (d, J=5.8 Hz, 1H), 8.82 (dd, J=10.4, 3.0 Hz, 1H), 8.76 (s, 1H), 8.02 (d, J=3.0 Hz, 1H), 7.90 (s, 1H), 7.73 (d, J=1.3 Hz, 1H), 7.60 (dd, J=8.6, 1.0 Hz, 1H), 7.37 (d, J=8.6 Hz, 1H), 6.66 (s, 2H), 4.67 (s, 1H), 3.78 (h, J=8.0 Hz, 1H), 2.26 (s, 4H), 2.18-2.08 (m, 1H), 1.37 (dt, J=14.7, 9.9 Hz, 2H), 1.13 (s, 3H).

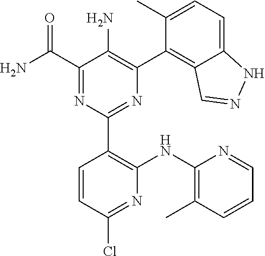 [SMILES: Cc1cccnc1Nc1nc(Cl)ccc1-c1nc(C(N)=O)c(N)c(-c2c(C)ccc3[nH]ncc23)n1]

### Compound 736 (5-amino-2-(6-chloro-2-((3-methylpyridin-2-yl)amino)pyridin-3-yl)-6-(5-methyl-1H-indazol-4-yl)pyrimidine-4-carboxamide)

Step 1. To a solution of ethyl 5-amino-2-(2-amino-6-chloro-3-pyridyl)-6-(5-methyl-1-tetrahydropyran-2-yl-indazol-4-yl)pyrimidine-4-carboxylate (60 mg, 118.1 μmol) in toluene (2 mL) were added 2-iodo-3-methyl-pyridine (30 mg, 137.0 μmol), Pd(OAc)₂ (3 mg, 13.4 μmol), cesium carbonate (77 mg, 236.3 μmol) and Xantphos (14 mg, 24.2 μmol). The mixture was degassed in vacuo, back-filled with N₂ and stirred at 110° C. for 8 h. The volatiles were removed in vacuo and the residue was purified by silica gel chromatography eluting with a gradient of EtOAc (40 to 100%) in heptane to provide ethyl 5-amino-2-[6-chloro-2-[(3-methyl-2-pyridyl)amino]-3-pyridyl]-6-(5-methyl-1-tetrahydropyran-2-yl-indazol-4-yl)pyrimidine-4-carboxylate (17 mg, 24% yield).

Step 2. A MW vessel was charged with ethyl 5-amino-2-[6-chloro-2-[(3-methyl-2-pyridyl)amino]-3-pyridyl]-6-(5-methyl-1-tetrahydropyran-2-yl-indazol-4-yl)pyrimidine-4-carboxylate (17 mg, 28.4 μmol), MeOH (1 mL) and ammonia (7 N solution in MeOH, 750 μL, 5.25 mmol). The vessel was sealed and the mixture was stirred at 80° C. for 5 h. The volatiles were removed in vacuo and the solid was dissolved in MeOH (1 mL). 4 M HCl solution in dioxane (150 μL, 0.6 mmol) was added and the mixture was stirred at 50° C. for 20 min. The volatiles were removed in vacuo and the residue was dissolved in MeOH, basified with Et₃N and concentrated to dryness. The residue was purified by preparative HPLC C18 column eluting with ACN/water/0.1% formic acid to provide 5-amino-2-[6-chloro-2-[(3-methyl-2-pyridyl)amino]-3-pyridyl]-6-(5-methyl-1H-indazol-4-yl)pyrimidine-4-carboxamide (6 mg, 44% yield). MS: [M+H]⁺: 486.2; ¹H NMR (400 MHz, DMSO-d6) δ 13.15 (s, 1H), 11.17 (s, 1H), 9.04 (d, J=8.2 Hz, 1H), 8.69 (d, J=2.1 Hz, 1H), 8.05 (dd, J=4.9, 1.8 Hz, 1H), 7.97 (d, J=2.1 Hz, 1H), 7.68 (s, 1H), 7.64-7.52 (m, 1H), 7.45 (dd, J=7.5, 1.7 Hz, 1H), 7.35 (d, J=8.6 Hz, 1H), 6.95 (dd, J=7.8, 4.5 Hz, 2H), 6.65 (s, 2H), 3.63-3.52 (m, 17H), 2.22 (s, 3H), 1.78-1.69 (m, 17H), 1.64 (s, 3H).

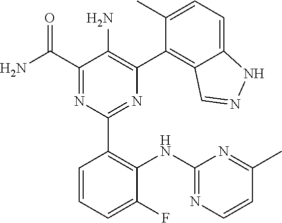 [SMILES: Cc1ccnc(Nc2c(F)cccc2-c2nc(C(N)=O)c(N)c(-c3c(C)ccc4[nH]ncc34)n2)n1]

### Compound 776 (5-amino-2-(3-fluoro-2-((4-methylpyrimidin-2-yl)amino)phenyl)-6-(5-methyl-1H-indazol-4-yl)pyrimidine-4-carboxamide)

Step 1. A MW vial was charged with Intermediate AL (300 mg, 447.5 μmol), Intermediate AV (152 mg, 538.8 μmol) and DMF (3 mL). N₂ was bubbled through the solution, then LiCl (42 mg, 990.8 μmol), CuI (18 mg, 94.5 μmol) Pd(dtbpf)Cl₂ (29 mg, 44.5 μmol) were added. The mixture was flushed with N₂, capped and transferred to a preheated (110° C.) heat block and stirred overnight. The cooled reaction mixture was diluted with aqueous saturated NH₄Cl solution, water and EtOAc and then filtered over a Celite™ plug. The layers of the filtrate were separated, and the aqueous layer was extracted with EtOAc (2×). The combined organic layers was washed with brine, dried over Na₂SO₄, filtered and adsorbed onto silica. The residue was purified by silica gel chromatography eluting with a gradient of EtOAc (0 to 100%) in heptane to provide ethyl 5-amino-2-[3-fluoro-2-[(4-methylpyrimidin-2-yl)amino]phenyl]-6-(5-methyl-1-tetrahydropyran-2-yl-indazol-4-yl)pyrimidine-4-carboxylate (36 mg, 14% yield). MS: [M+H]⁺: 583.3; Step 2. A MW vial containing ethyl 5-amino-2-[3-fluoro-2-[(4-methylpyrimidin-2-yl)amino]phenyl]-6-(5-methyl-1-tetrahydropyran-2-yl-indazol-4-yl)pyrimidine-4-carboxylate (36 mg, 61.8 μmol) and 7 N ammonia solution in MeOH (2 mL, 14 mmol) was capped and transferred to a preheated (80° C.) heat block for 20 min. The cooled reaction mixture was concentrated to dryness and the residue was dissolved in MeOH (1 mL) and 4 M HCl solution in dioxane (0.5 mL, 2.0 mmol) and the mixture was stirred at 45° C. for 15 min. The volatiles were removed in vacuo and the residue was dissolved in MeOH, basified with Et₃N and concentrated to dryness. The residue was purified by preparative HPLC C18 column eluting with ACN/water/0.1% formic acid to provide 5-amino-2-[3-fluoro-2-[(4-methylpyrimidin-2-yl)amino]phenyl]-6-(5-methyl-1H-indazol-4-yl)pyrimidine-4-carboxamide (15 mg, 52% yield). MS: [M+H]⁺: 470.3; ¹H NMR (400 MHz, DMSO-d6) δ 13.15 (s, 1H), 9.33 (s, 1H), 8.26 (d, J=2.7 Hz, 1H), 8.11 (d, J=4.9 Hz, 1H), 7.92-7.87 (m, 2H), 7.60 (d, J=8.2 Hz, 2H), 7.37 (d, J=8.6 Hz, 1H), 7.28 (dd, J=8.9, 3.8 Hz, 2H), 6.61 (d, J=5.0 Hz, 1H), 6.51 (br s, 2H), 2.21 (s, 3H), 2.19 (s, 3H).

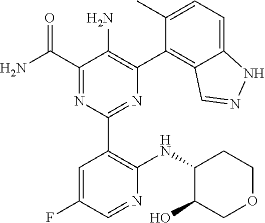 [SMILES: Cc1ccc2[nH]ncc2c1-c1nc(-c2cc(F)cnc2N[C@@H]2CCOC[C@H]2O)nc(C(N)=O)c1N]

### Compound 799 (5-amino-2-(5-fluoro-2-(((3S,4R)-3-hydroxytetrahydro-2H-pyran-4-yl)amino)pyridin-3-yl)-6-(5-methyl-1H-indazol-4-yl)pyrimidine-4-carboxamide)

Step 1. To a solution of 3-bromo-2,5-difluoro-pyridine (303 mg, 1.56 mmol) and K₂CO₃ (547 mg, 3.96 mmol) in DMSO (1 mL) was added (3S,4R)-4-aminotetrahydropyran-3-ol hydrochloride (245 mg, 1.59 mmol) at room temperature. The mixture was stirred at 140° C. for 110 min then poured into water and extracted with ether (3×). The combined organic extracts was washed with water and brine consecutively, dried over Na₂SO₄ and concentrated. The residue was purified by silica gel chromatography eluting with a gradient of EtOAc (0 to 100%) in heptane to provide (3S,4R)-4-[(3-bromo-5-fluoro-2-pyridyl)amino]tetrahydropyran-3-ol (267 mg, 59% yield). MS: [M+H]⁺: 293.0.

Step 2. A solution of Intermediate AL (204 mg, 304.3 μmol) and (3S,4R)-4-[(3-bromo-5-fluoro-2-pyridyl)amino]tetrahydropyran-3-ol (118 mg, 405.3 μmol) in DMF (1 mL) was bubbled through with N₂. Copper(I) iodide (16 mg, 84.0 μmol), bis(tri-tert-butylphosphine)palladium(0) (20 mg, 39.1 μmol) and LiCl (27 mg, 636.9 μmol) were added. The mixture was bubbled through again with N2 the stirred at 110° C. for 1 h under nitrogen. The cooled reaction mixture was diluted with EtOAc and aqueous saturated NH₄Cl solution and filtered over Celite™. The organic layer was separated and the aqueous layer was extracted with EtOAc (2×). The combine organic layers was washed with brine, dried over Na₂SO₄ and concentrated. The residue was purified by silica gel chromatography eluting with a gradient of EtOAc (50 to 100%) in heptane to provide ethyl 5-amino-2-[5-fluoro-2-[[(3S,4R)-3-hydroxytetrahydropyran-4-yl]amino]-3-pyridyl]-6-(5-methyl-1-tetrahydropyran-2-yl-indazol-4-yl)pyrimidine-4-carboxylate (76 mg, 42% yield). MS: [M+H]⁺: 592.3.

Step 3. A MW vial containing ethyl 5-amino-2-[5-fluoro-2-[[(3S,4R)-3-hydroxytetrahydropyran-4-yl]amino]-3-pyridyl]-6-(5-methyl-1-tetrahydropyran-2-yl-indazol-4-yl)pyrimidine-4-carboxylate (76 mg, 128.46 μmol) and 7 N ammonia solution in MeOH (3 mL, 21 mmol) was capped and transferred to a preheated (80° C.) heat block and stirred for 20 min. The reaction mixture was concentrated to dryness and the solid was dissolved in MeOH (2 mL) and 4 M HCl solution in dioxane (1 mL, 4 mmol). The mixture was stirred at 45° C. for 20 min then concentrated to dryness. The residue was purified by preparative HPLC C18 column eluting with ACN/water containing 10 mM ammonium bicarbonate to provide 5-amino-2-[5-fluoro-2-[[(3S,4R)-3-hydroxytetrahydropyran-4-yl]amino]-3-pyridyl]-6-(5-methyl-1H-indazol-4-yl)pyrimidine-4-carboxamide (20 mg, 33% yield). MS: [M+H]⁺: 479.3; ¹H NMR (400 MHz, DMSO-d6) δ 13.15 (s, 1H), 8.77 (s, 1H), 8.70 (s, 1H), 8.31 (d, J=13.1 Hz, 1H), 7.81 (s, 1H), 7.65 (s, 1H), 7.60 (d, J=8.5 Hz, 1H), 7.37 (d, J=8.5 Hz, 1H), 6.73 (d, J=7.6 Hz, 1H), 6.39 (s, 2H), 5.00 (d, J=5.3 Hz, 1H), 3.99 (br s, 1H), 3.90-3.70 (m, 2H), 3.70-3.45 (m, 1H), 3.04 (t, J=10.4 Hz, 1H), 2.24 (s, 3H), 1.95 (d, J=12.1 Hz, 1H), 1.64-1.39 (m, 1H).

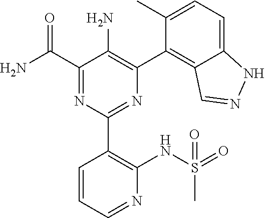 [SMILES: Cc1ccc2[nH]ncc2c1-c1nc(-c2cccnc2NS(C)(=O)=O)nc(C(N)=O)c1N]

### Compound 804 (5-amino-6-(5-methyl-1H-indazol-4-yl)-2-(2-(methylsulfonamido)pyridin-3-yl)pyrimidine-4-carboxamide)

To a solution of Intermediate BA (41 mg, 92.2 μmol) in THF (1 mL) were added Et₃N (55 μL, 394.6 μmol) and methanesulfonyl chloride (15 μL, 193.8 μmol). The mixture was stirred at 40° C. for 5 h.

The volatiles were removed in vacuo. To the residue was added water and the mixture was stirred at rt for 20 min and then filtered. The solid was washed with water, dried in vacuo. The crude product was dissolved in MeOH (1 mL) and 4 M HCl solution in dioxane (300 μL, 1.2 mmol). After stirring at 50° C. for for 20 min, the volatiles were removed in vacuo. The residue was dissolved in MeOH, basified with 7 N ammonia solution in MeOH, and concentrated to dryness. The residue was purified by preparative HPLC C18 column eluting with ACN/water/0.1% formic acid to provide 5-amino-6-(5-methyl-1H-indazol-4-yl)-2-(2-(methylsulfonamido)pyridin-3-yl)pyrimidine-4-carboxamide (15 mg, 37% yield). MS: [M+H]⁺: 439.3; ¹H NMR (400 MHz, DMSO-d6) δ 13.14 (s, 1H), 11.96 (s, 1H), 9.09 (dd, J=7.9, 1.9 Hz, 1H), 8.62 (s, 1H), 8.32 (dd, J=4.8, 1.8 Hz, 1H), 8.15-7.83 (m, 1H), 7.69 (s, 1H), 7.58 (d, J=8.6 Hz, 1H), 7.37 (d, J=8.6 Hz, 1H), 7.16 (dd, J=8.0, 4.8 Hz, 1H), 6.69 (s, 2H), 3.35 (s, 3H), 2.26 (s, 3H).

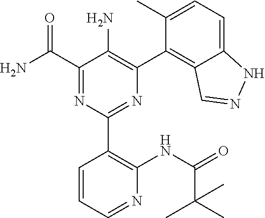 [SMILES: Cc1ccc2[nH]ncc2c1-c1nc(-c2cccnc2NC(=O)C(C)(C)C)nc(C(N)=O)c1N]

### Compound 805 (5-amino-6-(5-methyl-1H-indazol-4-yl)-2-(2-pivalamidopyridin-3-yl)pyrimidine-4-carboxamide)

To a solution of Intermediate BA (35 mg, 78.7 μmol) in THF (1 mL) were added Et₃N (45 μL, 322.86 μmol) and 2,2-dimethylpropanoyl chloride (20 μL, 163.38 μmol) at rt. The mixture was stirred at 40° C. for 5 h. The volatiles were removed in vacuo. To the residue was added water and the mixture was stirred at rt for 20 min and then filtered. The solid was washed with water, dried in vacuo. The crude product was dissolved in MeOH (1 mL) and 4 M HCl solution in dioxane (300 μL, 1.2 mmol). After stirring at 50° C. for for 20 min, the volatiles were removed in vacuo. The residue was dissolved in MeOH, basified with 7 N ammonia solution in MeOH, concentrated to dryness and then purified by preparative HPLC C18 column eluting with ACN/water/0.1% formic acid to provide 5-amino-2-[2-(2,2-dimethylpropanoylamino)-3-pyridyl]-6-(5-methyl-1H-indazol-4-yl)pyrimidine-4-carboxamide (20 mg, 57% yield). MS: [M+H]⁺: 445.3; ¹H NMR (400 MHz, DMSO-d6) δ 13.17 (s, 1H), 11.65 (s, 1H), 9.03 (dd, J=7.9, 2.0 Hz, 1H), 8.70 (d, J=2.2 Hz, 1H), 8.37 (dd, J=4.7, 1.9 Hz, 1H), 7.99 (d, J=2.2 Hz, 1H), 7.71 (s, 1H), 7.69-7.59 (m, 1H), 7.38 (d, J=8.6 Hz, 1H), 7.23 (dd, J=7.9, 4.7 Hz, 1H), 6.70 (s, 2H), 2.23 (s, 3H), 0.61 (s, 9H).

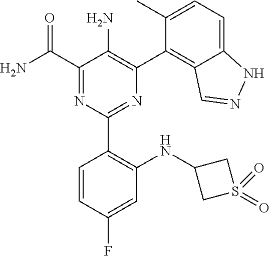 [SMILES: Cc1ccc2[nH]ncc2c1-c1nc(-c2ccc(F)cc2NC2CS(=O)(=O)C2)nc(C(N)=O)c1N]

### Compound 811 (5-amino-2-(2-((1,1-dioxidothietan-3-yl)amino)-4-fluorophenyl)-6-(5-methyl-1H-indazol-4-yl)pyrimidine-4-carboxamide)

Step 1. A solution of Intermediate AB (60.0 mg, 144 μmol) and Intermediate BB (125 mg, 241 μmol) in dioxane (975 μL) was placed in a round bottom flask. The mixture was bubbled with a nitrogen balloon for 10 min before adding Pd(dtbpf)Cl₂ (16.0 mg, 24.1 μmol) and sodium bicarbonate (2 M in water, 241 μL, 482 μmol). The reaction mixture was heated to 100° C. for 2 h. Upon cooling to rt, the reaction mixture was diluted with EtOAc, filtered through Celite™, washed with EtOAc and concentrated. The residue was purified by silica gel chromatography eluting with a gradient of EtOAc (0 to 70%) in hexanes to provide ethyl 5-amino-2-(2-((1,1-dioxidothietan-3-yl)amino)-5-fluorophenyl)-6-(5-methyl-1-(tetrahydro-2H-pyran-2-yl)-1H-indazol-4-yl)pyrimidine-4-carboxylate (60 mg, 70% yield).

Step 2. Ethyl 5-amino-2-(2-((1,1-dioxidothietan-3-yl)amino)-5-fluorophenyl)-6-(5-methyl-1-(tetrahydro-2H-pyran-2-yl)-1H-indazol-4-yl)pyrimidine-4-carboxylate (30.4 mg, 52.3 μmol) was placed in microwave reaction vial and ammonia solution solution (7 N in MeOH, 747 μL, 5.23 mmol). was added. The reaction mixture was heated to 85° C. for 5 h. Upon cooling to rt, the volatiles were removed in vacuo. The solid was dissolved in 1.25 M HCl solution in EtOH (1.70 mL, 2.12 mmol) and the reaction mixture was stirred 50° C. for 1 h. Upon cooling to rt, the reaction mixture was quenched with aqueous saturated NaHCO₃. The mixture was extracted with EtOAc (2×) and the organic layers were combined, washed with brine, dried over Na₂SO₄, filtered and concentrated in vacuo. The residue was purified by preparative HPLC C18 column eluting with ACN/water/0.1% formic acid to provide 5-amino-2-(2-((1,1-dioxidothietan-3-yl)amino)-5-fluorophenyl)-6-(5-methyl-1H-indazol-4-yl)pyrimidine-4-carboxamide (17 mg, 66% yield). MS: [M+H]⁺: 482.1; ¹H NMR (400 MHz, DMSO-d6) δ 13.17 (s; 1H); 9.21 (d; J=5.30 Hz; 1H); 8.51 (t; J=7.97 Hz; 1H); 8.42 (s; 1H); 7.91 (s; 1H); 7.68 (s; 1H); 7.61 (d; J=8.55 Hz; 1H); 7.37 (d; J=8.58 Hz; 1H); 6.47-6.54 (m; 3H); 6.37 (d; J=11.96 Hz; 1H); 4.54-4.63 (m; 2H); 4.23 (s; 1H); 3.65 (d; J=13.63 Hz; 1H); 3.40 (d; J=13.69 Hz; 1H); 2.25 (s; 3H).

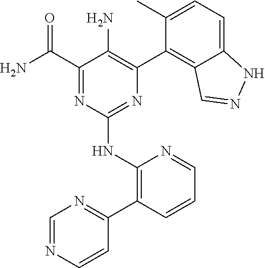 [SMILES: Cc1ccc2[nH]ncc2c1-c1nc(Nc2ncccc2-c2ccncn2)nc(C(N)=O)c1N]

### Compound 818 (5-amino-6-(5-methyl-1H-indazol-4-yl)-2-((3-(pyrimidin-4-yl)pyridin-2-yl)amino)pyrimidine-4-carboxamide)

Step 1. To a solution of 4-(2-chloropyridin-3-yl)pyrimidine (921 mg, 4.81 mmol) in NMP (4 mL) were added 4-methoxybenzylamine (950 μL, 7.27 mmol) and K₂CO₃ (1.5 g, 10.9 mmol). The suspension was heated to 125° C. for 6 h. The cooled reaction mixture was partitioned between water-brine (3:1, 40 mL) and ethyl acetate. The product was extracted with ethyl acetate (3×) and the combined organic phases was washed with water (3×) and brine, dried over Na₂SO₄ and evaporated. The residue was purified by silica gel chromatography eluting with a gradient of EtOAc (5 to 65%) in heptane to provide N-(4-methoxybenzyl)-3-(pyrimidin-4-yl)pyridin-2-amine (1.33 g, 95% yield). ¹H NMR (400 MHz, DMSO-d6) δ 3.72 (s, 3H), 4.68 (d, J=5.6 Hz, 2H), 6.72 (dd, J=7.7, 4.8 Hz, 1H), 6.80-6.94 (m, 2H), 7.28 (d, J=8.8 Hz, 2H), 8.12 (dd, J=5.7, 1.3 Hz, 1H), 8.22 (dd, J=4.6, 1.7 Hz, 1H), 8.28 (dd, J=7.8, 1.7 Hz, 1H), 8.82 (d, J=5.6 Hz, 1H), 9.19 (d, J=1.2 Hz, 1H), 9.67 (t, J=5.6 Hz, 1H).

Step 2. A RBF was charged with N-(4-methoxybenzyl)-3-(pyrimidin-4-yl)pyridin-2-amine (1.31 g, 4.48 mmol) and L-cysteine (1.63 g, 13.4 mmol). Trifluoroacetic acid (15 mL, 195.9 mmol) was added and the reaction mixture was stirred to 60° C. for 4 h then a at rt for 3 h. The solvent was evaporated and the crude residue was partitioned between ethyl acetate basicified with aqueous solution of sodium hydroxide at pH>10-11. The aqueous phase was back extracted with ethyl acetate (3×) and the combined organic phases were washed with a diluted solution of sodium hydroxide, water and brine, dried over Na₂SO₄, filtered and concentrated. The residue was purified by silica gel chromatography eluting with a gradient of MeOH (1 to 5%) in EtOAc to provide 3-(pyrimidin-4-yl)pyridin-2-amine (591 mg, 77% yield). MS: [M+H]⁺: 173.2. ¹H NMR (400 MHz, DMSO-d6) δ 6.70 (dd, J=7.6, 4.6 Hz, 1H), 7.69 (br. s, 2H), 8.06 (d, J=5.4 Hz, 1H), 8.10-8.17 (m, 1H), 8.21 (d, J=7.6 Hz, 1H), 8.81 (d, J=5.6 Hz, 1H), 9.22 (s, 1H).

Step 3. A vial was charged with 3-(pyrimidin-4-yl)pyridin-2-amine (77.9 mg, 0.4521 mmol) and degassed dioxane (3 mL). Sodium tert-butoxide (86.9 mg, 0.904 mmol) was added and the solution was stirred 5 minutes. Intermediate L (91.2 mg, 0.301 mmol), 2-(dicyclohexylphosphino)_{3,6}-dimethoxy-2′,4′,6′-triisopropyl-1,1′-biphenyl (16.2 mg, 0.0301 mmol) and BrettPhos Pd G3 (27.3 mg, 0.0301 mmol) were added and the suspension was purged with nitrogen and stirred at 105° C. overnight. More sodium tert-Butoxide (50 mg, 0.52 mmol) and Brettphos Pd G3 (20 mg, 0.022 mmol) were added to the reaction mixture and the mixture was stirred at 105° C. 6 h. The cooled reaction mixture was diluted in ethyl acetate and was washed with a dilute aqueous solution of ammonium chloride, water and brine, dried over sodium sulfate and evaporated. The residue was purified by silica gel chromatography eluting with a gradient of MeOH (5 to 15%) in DCM and then repurified by C18 silica gel chromatography eluting with a gradient of MeOH (5 to 50%) in water to provide 5-amino-6-(5-methyl-1H-indazol-4-yl)-2-((3-(pyrimidin-4-yl)pyridin-2-yl)amino)pyrimidine-4-carboxamide (9.0 mg, 7% yield). MS: [M+H]⁺: 439.2; ¹H NMR (400 MHz, DMSO-d6) δ 2.22 (s, 3H), 5.89 (s, 2H), 7.12 (dd, J=7.7, 4.8 Hz, 1H), 7.34 (d, J=8.6 Hz, 1H), 7.57 (d, J=8.6 Hz, 1H), 7.67 (s, 1H), 7.79 (s, 2H), 8.02 (dd, J=5.6, 1.2 Hz, 1H), 8.28 (dd, J=7.8, 1.7 Hz, 1H), 8.39 (dd, J=4.8, 1.8 Hz, 1H), 8.81 (d, J=5.4 Hz, 1H), 9.24 (d, J=1.0 Hz, 1H), 10.92 (s, 1H), 13.11 (s, 1H).

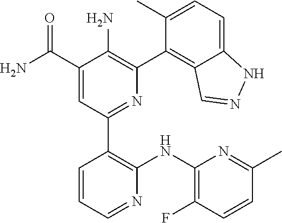 [SMILES: Cc1ccc(F)c(Nc2ncccc2-c2cc(C(N)=O)c(N)c(-c3c(C)ccc4[nH]ncc34)n2)n1]

### Compound 827 (5-amino-2′-((3-fluoro-6-methylpyridin-2-yl)amino)-6-(5-methyl-1H-indazol-4-yl)-[2,3′-bipyridine]-4-carboxamide)

Step 1. Intermediate BT (220 mg, 0.53 mmol), 3-(4,4,5,5-tetramethyl-1,3,2-dioxaborolan-2-yl)pyridin-2-amine (117 mg, 0.53 mmol), PdCl₂(dppf) (39.6 mg, 0.053 mmol), potassium carbonate (220 mg, 1.59 mmol), dioxane (2.7 mL) and water (0.80 mL) were added to a vial fitted with a stir bar. The mixture was degassed with nitrogen 2 min, the vial sealed and heated to 90° C. for 0.5 h with stirring. The reaction was cooled to ambient temperature and filtered through Celite™ with EtOAc as eluent. The filtrate was adsorbed onto silica gel and the residue was purified by silica gel chromatography eluting with a gradient of EtOAc (2 to 100%) in heptane to provide ethyl-2′,5-diamino-6-(5-methyl-1-(tetrahydro-2H-pyran-2-yl)-1H-indazol-4-yl)-[2,3′-bipyridine]-4-carboxylate (210 mg, 84% yield). MS: [M+H]⁺: 473.2.

Step 2. Ethyl-2′,5-diamino-6-(5-methyl-1-(tetrahydro-2H-pyran-2-yl)-1H-indazol-4-yl)-[2,3′-bipyridine]-4-carboxylate (210 mg, 0.444 mmol), 2-bromo-3-fluoropyridine (45.9 μL, 0.444 mmol), cesium carbonate (443 mg, 1.33 mmol), XantPhos Pd G2 (40.7 mg, 0.0444 mmol) and toluene (4.2 mL) were added to a vial fitted with a stir bar. The mixture was degassed with nitrogen 5 min, the vial was sealed and heated to 90° C. for 4 h with stirring. The reaction was cooled to ambient temperature and filtered through Celite™ with EtOAc as eluent. The filtrate was adsorbed onto silica and the residue was purified by silica gel chromatography eluting with a gradient of EtOAc (2 to 100%) in heptane to provide ethyl-5-amino-2′-((3-fluoropyridin-2-yl)amino)-6-(5-methyl-1-(tetrahydro-2H-pyran-2-yl)-1H-indazol-4-yl)-[2,3′-bipyridine]-4-carboxylate (180 mg, 71% yield). MS: [M+H]⁺: 568.2.

Step 3. Ethyl-5-amino-2′-((3-fluoropyridin-2-yl)amino)-6-(5-methyl-1-(tetrahydro-2H-pyran-2-yl)-1H-indazol-4-yl)-[2,3′-bipyridine]-4-carboxylate (180 mg, 0.317 mmol) and ammonia solution (7 N in MeOH, 5.0 mL, 35 mmol) were added to a pressure tube fitted with a stir bar. The tube was sealed and heated to 90° C. for 18 h with stirring. The reaction was cooled to ambient temperature and concentrated to dryness in vacuo. The residue was taken up in MeOH (2.0 mL) and a solution of HCl (4 M in dioxane, 0.92 mL, 3.68 mmol) was added. The reaction was stirred at 50° C. for 30 min, cooled to ambient temperature and concentrated in vacuo. The residue was purified by preparative HPLC eluting with a gradient of acetonitrile in water containing 10 mM ammonium bicarbonate (pH adjusted to 10 with NH₄OH) to provide 5-amino-2′-((3-fluoro-6-methylpyridin-2-yl)amino)-6-(5-methyl-1H-indazol-4-yl)-[2,3′-bipyridine]-4-carboxamide (21.0 mg, 15%). MS: [M+H]⁺: 455.2; ¹H NMR (400 MHz, DMSO-d6) δ 13.05 (s, 1H), 10.88 (s, 1H), 8.41 (s, 1H), 8.24 (dd, J=7.7, 1.6 Hz, 1H), 8.17 (s, 1H), 8.06 (dd, J=4.8, 1.5 Hz, 1H), 7.95 (d, J=4.8 Hz, 1H), 7.74 (s, 1H), 7.62-7.44 (m, 3H), 7.35 (d, J=8.6 Hz, 1H), 7.09-6.95 (m, 2H), 6.15 (s, 2H), 2.19 (s, 3H).

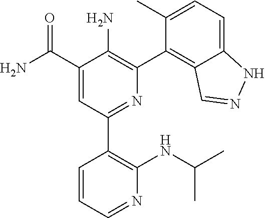 [SMILES: Cc1ccc2[nH]ncc2c1-c1nc(-c2cccnc2NC(C)C)cc(C(N)=O)c1N]

### Compound 866 (5-amino-2′-(isopropylamino)-6-(5-methyl-1H-indazol-4-yl)-[2,3′-bipyridine]-4-carboxamide)

Step 1. Intermediate BT (75 mg, 0.181 mmol), (2-fluoropyridin-3-yl)boronic acid (76.4 mg, 0.542 mmol), XPhos Pd G2 (15.3 mg, 0.0181 mmol), potassium carbonate (75 mg, 0.542 mmol), dioxane (2 mL) and water (0.5 mL) were added to a vial fitted with a stir bar. The mixture was degassed with nitrogen 5 min, the vial sealed and heated to 90° C. for 1 h with stirring. The reaction was cooled to ambient temperature, filtered through Celite™ with EtOAc as eluent and the filtrate was adsorbed onto silica. The residue was purified by silica gel chromatography eluting with a gradient of EtOAc (3 to 100%) in heptane to provide ethyl-5-amino-2′-fluoro-6-(5-methyl-1-(tetrahydro-2H-pyran-2-yl)-1H-indazol-4-yl)-[2,3′-bipyridine]-4-carboxylate (60.0 mg, 70% yield). MS: [M+H]⁺: 476.3.

Step 2. Ethyl-5-amino-2′-fluoro-6-(5-methyl-1-(tetrahydro-2H-pyran-2-yl)-1H-indazol-4-yl)-[2,3′-bipyridine]-4-carboxylate (60.0 mg, 0.126 mmol), isopropylamine (37.3 mg, 0.631 mmol), DIPEA (85.1 mg, 0.631 mmol) and NMP (1.5 mL) were added to a microwave vial fitted with a stir bar. The vial was sealed and placed in a microwave reactor at 150° C. for 4 h. The reaction was cooled to ambient temperature, diluted with EtOAc and washed with water and brine. The organic portion was separated and dried over sodium sulfate, filtered and concentrated in vacuo affording ethyl-5-amino-2′-(isopropylamino)-6-(5-methyl-1-(tetrahydro-2H-pyran-2-yl)-1H-indazol-4-yl)-[2,3′-bipyridine]-4-carboxylate (60 mg, 92% yield) which was used without further purification. MS: [M+H]⁺: 515.3.

Step 3. Ethyl-5-amino-2′-(isopropylamino)-6-(5-methyl-1-(tetrahydro-2H-pyran-2-yl)-1H-indazol-4-yl)-[2,3′-bipyridine]-4-carboxylate (60 mg, 0.117 mmol) and 7 N ammonia solution in MeOH (4 mL, 28 mmol) were added to a pressure tube fitted with a stir bar. The tube was sealed and heated to 80° C. for 18 h with stirring. The reaction was cooled to ambient temperature and concentrated to dryness in vacuo. The residue was taken up in MeOH (2.0 mL) and a solution of 4 M HCl solution in dioxane (0.3 mL, 1.2 mmol) was added. The reaction was stirred at 50° C. for 30 min, cooled to ambient temperature and concentrated in vacuo. The residue was purified by preparative HPLC eluting with a gradient of acetonitrile in water containing 10 mM ammonium bicarbonate (pH adjusted to 10 with NH₄OH) to provide 5-amino-2′-(isopropylamino)-6-(5-methyl-1H-indazol-4-yl)-[2,3′-bipyridine]-4-carboxamide (15.7 mg, 34%). MS: [M+H]⁺: 402.1; ¹H NMR (DMSO-d6) δ 13.08 (s, 1H), 8.93 (d, J=6.9 Hz, 1H), 8.39 (s, 1H), 8.10 (s, 1H), 8.07 (dd, J=7.6, 1.6 Hz, 1H), 7.97 (dd, J=4.7, 1.5 Hz, 1H), 7.70 (s, 1H), 7.54 (d, J=6.3 Hz, 2H), 7.34 (d, J=8.7 Hz, 1H), 6.58 (dd, J=7.6, 4.8 Hz, 1H), 6.09 (s, 2H), 4.03 (dq, J=13.0, 6.5 Hz, 1H), 2.22 (s, 3H), 0.84 (d, J=6.4 Hz, 3H), 0.73 (d, J=6.5 Hz, 3H).

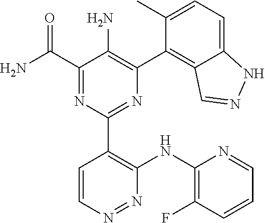 [SMILES: Cc1ccc2[nH]ncc2c1-c1nc(-c2ccnnc2Nc2ncccc2F)nc(C(N)=O)c1N]

### Compound 886 (5-amino-2-(3-((3-fluoropyridin-2-yl)amino)pyridazin-4-yl)-6-(5-methyl-1H-indazol-4-yl)pyrimidine-4-carboxamide)

Step 1. In a sealed tube, cesium fluoride (143 mg, 935 μmol), copper(I) iodide (14.2 mg, 74.8 μmol), Intermediate AL (250 mg, 374 μmol) and 3-amino-4-bromo-6-chloropyridazine (117 mg, 546 μmol) were dissolved in DMF (2.49 mL). The suspension was degassed with nitrogen 10 minutes and P(tBu)₃ Pd G2 (19.2 mg, 37.4 μmol) was added. The vial was sealed and heated to 100° C. for 3 h with stirring. Silica was added and the reaction was evaporated. The residue was purified by silica gel chromatography eluting with a gradient of EtOAc (0 to 80%) in DCM to provide ethyl 5-amino-2-(3-amino-6-chloropyridazin-4-yl)-6-(5-methyl-1-(tetrahydro-2H-pyran-2-yl)-1H-indazol-4-yl)pyrimidine-4-carboxylate (76.0 mg, 40% yield).

Step 2. Palladium on carbon (15.9 mg, 14.9 μmol) was dissolved in MeOH (1 mL) and a solution of ethyl-5-amino-2-(3-amino-6-chloropyridazin-4-yl)-6-(5-methyl-1-(tetrahydro-2H-pyran-2-yl)-1H-indazol-4-yl)pyrimidine-4-carboxylate (76.0 mg, 149 μmol) in MeOH (700 μL) was added. The mixture was purged with hydrogen and stirred for 12 hours at rt under 1 atm of hydrogen. The reaction was then purged with nitrogen, filtered through Celite™ and washed with MeOH. The filtrate was concentrated to afford ethyl 5-amino-2-(3-aminopyridazin-4-yl)-6-(5-methyl-1-(tetrahydro-2H-pyran-2-yl)-1H-indazol-4-yl)pyrimidine-4-carboxylate (65 mg, 92%). MS: [M+H]⁺: 475.2.

Step 3. In a sealed tube, ethyl 5-amino-2-(3-aminopyridazin-4-yl)-6-(5-methyl-1-(tetrahydro-2H-pyran-2-yl)-1H-indazol-4-yl)pyrimidine-4-carboxylate (65.0 mg, 137 μmol), 2-bromo-3-fluoropyridine (24.1 mg, 134 μmol) and cesium carbonate (137 mg, 411 μmol) were dissolved in toluene (5 mL) and DMSO (685 μL). The mixture was degassed with nitrogen 10 minutes and XantPhos Pd G2 (12.6 mg, 13.7 μmol was added. The tube was sealed and heated to 90° C. with stirring for 8 h. The reaction was cooled to ambient temperature and adsorbed onto silica. The residue was purified by silica gel chromatography eluting with a gradient of i-PrOH (5 to 100%) in DCM to provide a solid which was dissolved in MeOH (200 μL). 7 N ammonia solution in MeOH (100 μL, 700 μmol) was added and the mixture was heated at 80° C. for 2 h. The cooled reaction mixture was concentrated and dissolved in MeOH (200 μL) and 4 M HCl solution in dioxane (58 μL, 232 μmol) was added and the solution was heated at 50° C. for 2 h. The mixture was concentrated and the residue was purified by preparative HPLC eluting with a gradient of acetonitrile in water containing 10 mM ammonium bicarbonate (pH adjusted to 10 with NH₄OH) to provide 5-amino-2-(3-((3-fluoropyridin-2-yl)amino)pyridazin-4-yl)-6-(5-methyl-1H-indazol-4-yl)pyrimidine-4-carboxamide (2 mg, 3%). MS: [M+H]⁺: 457.1; ¹H NMR (DMSO-d6) 13.13 (s, 1H), 10.77 (s, 1H), 8.89 (d, J=5.1 Hz, 1H), 8.73 (d, J=5.1 Hz, 2H), 8.09-7.93 (m, 2H), 7.71 (s, 1H), 7.62 (dd, J=13.4, 5.0 Hz, 2H), 7.37 (d, J=8.6 Hz, 1H), 7.22-7.09 (m, 1H), 6.88 (br s, 2H), 2.26 (s, 3H). MS (ES+) m/z 457.1 (MH+)

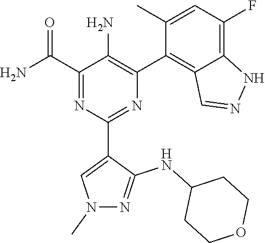 [SMILES: Cc1cc(F)c2[nH]ncc2c1-c1nc(-c2cn(C)nc2NC2CCOCC2)nc(C(N)=O)c1N]

### Compound 903 (5-amino-6-(7-fluoro-5-methyl-1H-indazol-4-yl)-2-(1-methyl-3-((tetrahydro-2H-pyran-4-yl)amino)-1H-pyrazol-4-yl)pyrimidine-4-carboxamide)

Step 1. A MW vessel was charged with Intermediate BJ (166 mg, 540.4 μmol), dioxane (2 mL), Intermediate BP (150 mg, 345.7 μmol) and 2 M aqueous solution of K₂CO₃ (360 μL, 0.72 mmol). Pd(dppf)Cl₂·DCM (15 mg, 18.37 μmol) was added and the vessel was flushed with N₂, sealed and stirred 105° C. for 1 h. The cooled reaction mixture was acidified with 10% aqueous HCl solution then treated with 1 M solution (trimethylsilyl)diazomethane in hexanes (1 ml, 1 mmol). The mixture was stirred for 30 min at rt and the reaction mixture was diluted with EtOAc and water. The aqueous layer was cut and the organic layer was washed with brine, dried over MgSO₄, filtered and concentrated. The residue was purified by silica gel chromatography eluting with a gradient of EtOAc (50 to 100%) in heptane to provide a mixture of methyl and ethyl 5-amino-6-(7-fluoro-5-methyl-1-tetrahydropyran-2-yl-indazol-4-yl)-2-[1-methyl-3-(tetrahydropyran-4-ylamino)pyrazol-4-yl]pyrimidine-4-carboxylate (109 mg, 54% yield). MS: [M+H]⁺: 565.0/579.0.

Step 2. A 2-5 mL MW vial was charged with the mixture of methyl and ethyl 5-amino-6-(7-fluoro-5-methyl-1-tetrahydropyran-2-yl-indazol-4-yl)-2-[1-methyl-3-(tetrahydropyran-4-ylamino)pyrazol-4-yl]pyrimidine-4-carboxylate (109 mg, 188.4 μmol), MeOH (1 mL) and 7 N ammonia solution in MeOH (1 mL, 7 mmol). The vessel was sealed and stirred at 85° C. for 4 h. The reaction mixture was concentrated then redissolved in DCM (1.5 mL) and TFA (0.5 mL, 6.49 mmol). The mixture was aged at rt for 2 h then concentrated, redissolved in DMSO, basicified with a few drops of Et₃N and purified by preparative HPLC eluting with a gradient of acetonitrile in water containing 10 mM ammonium bicarbonate (pH adjusted to 10 with NH₄OH) to provide 5-amino-6-(7-fluoro-5-methyl-1H-indazol-4-yl)-2-[1-methyl-3-(tetrahydropyran-4-ylamino)pyrazol-4-yl]pyrimidine-4-carboxamide (22 mg, 25% yield). MS: [M+H]⁺: 465.9; ¹H NMR (DMSO-d6) δ 13.71 (s, 1H), 8.44 (s, 1H), 8.24 (s, 1H), 7.84 (s, 1H), 7.76 (dd, J=3.5, 1.5 Hz, 1H), 7.27 (d, J=12.0 Hz, 1H), 6.24 (s, 2H), 5.98 (d, J=7.7 Hz, 1H), 3.66 (s, 3H), 3.65-3.58 (m, 2H), 3.58-3.48 (m, 1H), 3.31-3.24 (m, 2H), 2.26 (s, 3H), 1.92-1.78 (m, 2H), 1.22-1.05 (m, 2H).

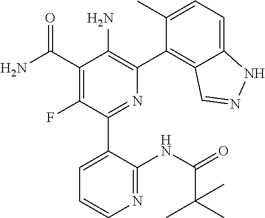 [SMILES: Cc1ccc2[nH]ncc2c1-c1nc(-c2cccnc2NC(=O)C(C)(C)C)c(F)c(C(N)=O)c1N]

### Compound 905 (5-amino-3-fluoro-6-(5-methyl-1H-indazol-4-yl)-2′-pivalamido-[2,3′-bipyridine]-4-carboxamide)

Step 1. 2,6-Dichloro-5-fluoropyridin-3-amine (500 mg, 2.71 mmol) was dissolved in DMF (13.5 mL) and sodium hydride (60% dispersion in mineral oil, 271 mg, 6.77 mmol) was added portion wise at room temperature. 4-Methoxybenzyl chloride (936 μL, 6.77 mmol) was added and the mixture was stirred overnight at rt. The reaction was quench with water and extracted 3× with DCM. The organic phases were combined and adsorbed onto silica. The residue was purified by silica gel chromatography eluting with a gradient of EtOAc (0 to 50%) in heptane to provide 2,6-dichloro-5-fluoro-N,N-bis(4-methoxybenzyl)pyridin-3-amine (750 mg, 66% yield). ¹H NMR (400 MHz, Chloroform-d) δ 7.23-7.12 (m, 4H), 6.96 (d, J=8.8 Hz, 1H), 6.89-6.78 (m, 4H), 4.17 (s, 4H), 3.79 (s, 6H).

Step 2. In a flame-dried RBF, 2,6-dichloro-5-fluoro-N,N-bis(4-methoxybenzyl)pyridin-3-amine (600 mg, 1.42 mmol) was dissolved in THF (7.1 mL) and cooled to −78° C. 1 M Lithium diisopropylamide solution in THF (1.42 mL, 1.42 mmol) was added dropwise and the mixture was stirred for 1 hour at −78° C. Ethyl chloroformate (280 μL, 2.85 mmol) in THF (2 mL) was added dropwise and the mixture was stirred for another hour. The reaction was allowed to warm-up to rt and then quenched with MeOH. Silica was added and the mixture was concentrated. The residue was purified by silica gel chromatography eluting with a gradient of EtOAc (0 to 50%) in heptane to provide ethyl 3-(bis(4-methoxybenzyl)amino)-2,6-dichloro-5-fluoroisonicotinate (450 mg, 64% yield).

Step 3. In a vial fitted with a stir bar, ethyl 3-(bis(4-methoxybenzyl)amino)-2,6-dichloro-5-fluoroisonicotinate (450 mg, 912 μmol), potassium carbonate (378 mg, 2.74 mmol) and 5-methyl-1 h-indazole-4-boronic acid (261 mg, 1.00 mmol) were dissolved in dioxane (3.70 mL) and water (1.0 mL). The mixture was degassed 10 minutes with nitrogen and Pd(PPh₃)₄ (52.7 mg, 45.1 μmol) was added. The vial was sealed and stirred at 90° C. for 24 h with stirring. Silica was added to the cooled reaction mixture and concentrated. The residue was purified by silica gel chromatography eluting with a gradient of EtOAc (0 to 80%) in heptane to provide ethyl 3-(bis(4-methoxybenzyl)amino)-2-chloro-5-fluoro-6-(5-methyl-1-(tetrahydro-2H-pyran-2-yl)-1H-indazol-4-yl)isonicotinate (330 mg, 54% yield). MS: [M+H]⁺: 673.4.

Step 4. Ethyl 3-(bis(4-methoxybenzyl)amino)-2-chloro-5-fluoro-6-(5-methyl-1-(tetrahydro-2H-pyran-2-yl)-1H-indazol-4-yl)isonicotinate (330 mg, 490 μmol) was dissolved in DCM (1 mL) and trifluoroacetic acid (948 μL, 12.3 mmol) was added. The mixture was heated at 90° C. for 1 h with stirring. The mixture was evaporated off and co-evaporated with toluene to afford ethyl 3-amino-2-chloro-5-fluoro-6-(5-methyl-1H-indazol-4-yl)isonicotinate (145 mg, 85% yield). MS: [M+H]⁺: 349.1.

Step 5. In a vial fitted with a stir bar, ethyl 3-amino-2-chloro-5-fluoro-6-(5-methyl-1H-indazol-4-yl)isonicotinate (100 mg, 287 μmol), N-(3-(4,4,5,5-tetramethyl-1,3,2-dioxaborolan-2-yl)pyridin-2-yl)pivalamide (131 mg, 430 μmol) and potassium carbonate (119 mg, 860 μmol) were dissolved in dioxane (1.15 mL) and water (287 μL)_The mixture was degassed 10 min with nitrogen and XPhos Pd G3 (24.5 mg, 28.1 μmol) was added. The vial was sealed and heated to 80° C. for 24 h with stirring. Silica was added to the cooled reaction mixture and concentrated. The residue was purified by silica gel chromatography eluting with a gradient of EtOAc (0 to 80%) in heptane to provide ethyl 3-amino-5-fluoro-6-(5-methyl-1H-indazol-4-yl)-2-(2-pivalamidophenyl)isonicotinate (50 mg, 36% yield). MS: [M+H]⁺: 491.3.

Step 6. In a sealed tube, ethyl 3-amino-5-fluoro-6-(5-methyl-1H-indazol-4-yl)-2-(2-pivalamidophenyl)isonicotinate (50.0 mg, 102 μmol) was dissolved in MeOH (510 μL) and 7 N ammonia solution in MeOH (728 μL, 5.10 mmol) was added. The mixture was heated at 80° C. for 12 h. The mixture was concentrated and the residue was purified by preparative HPLC eluting with a gradient of acetonitrile in water containing 10 mM ammonium bicarbonate (pH adjusted to 10 with NH₄OH) to provide 3-amino-5-fluoro-6-(5-methyl-1H-indazol-4-yl)-2-(2-pivalamidophenyl)-isonicotinamide (3.3 mg, 7% yield). MS: [M+H]⁺: 462.2; ¹H NMR (400 MHz, DMSO-d6) δ 12.91 (s, 1H), 9.54 (s, 1H), 8.53-8.44 (m, 1H), 8.02 (s, 1H), 7.97 (s, 1H), 7.87 (dd, J=7.6, 1.7 Hz, 1H), 7.80 (s, 1H), 7.44 (d, J=8.6 Hz, 1H), 7.37 (dd, J=7.6, 4.8 Hz, 1H), 7.25 (d, J=8.5 Hz, 1H), 5.58 (s, 2H), 2.24 (s, 3H), 0.96 (s, 9H). MS (ES+) m/z 462.2 (MH+)

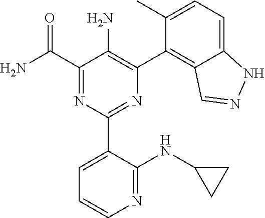 [SMILES: Cc1ccc2[nH]ncc2c1-c1nc(-c2cccnc2NC2CC2)nc(C(N)=O)c1N]

### Compound 914 (5-amino-2-(2-(cyclopropylamino)pyridin-3-yl)-6-(5-methyl-1H-indazol-4-yl)pyrimidine-4-carboxamide)

A MW vial was charged with 3-bromo-N-cyclopropyl-pyridin-2-amine (127 mg, 596.0 μmol in dioxane (3 mL). Nitrogen was bubbled through the solution and bis(pinacolato)diboron (194 mg, 764.0 μmol), KOAc (150 mg, 1.53 mmol) and Pd(dppf)Cl₂·DCM (27 mg, 33.1 μmol) were added. The vial was flushed with N₂, sealed and stirred at 110° C. for 1 h. Intermediate J (150 mg, 495.5 μmol) was added followed by 2 M aqueous solution of K₂CO₃ (620 μL, 1.24 mmol) and more Pd(dppf)Cl₂·DCM (32 mg, 39.2 μmol). The vial was flushed with N₂, sealed and stirred at 110° C. for 90 min. The cooled reaction mixture was partitioned between water and DCM. Layers were separated, the aqueous layer was extracted with DCM (2×) and the combined organic extracts was concentrated. The residue was purified by preparative HPLC C18 column eluting with ACN/water/0.1% formic acid to provide 5-amino-2-[2-(cyclopropylamino)-3-pyridyl]-6-(5-methyl-1H-indazol-4-yl)pyrimidine-4-carboxamide (17 mg, 9% yield). MS: [M+H]⁺: 401.0; ¹H NMR (400 MHz, DMSO-d6) δ 13.21 (s, 1H), 9.03 (d, J=3.6 Hz, 1H), 8.82 (dd, J=7.7, 1.9 Hz, 1H), 8.59 (d, J=2.3 Hz, 1H), 8.14 (dd, J=4.7, 1.9 Hz, 1H), 7.93 (d, J=2.2 Hz, 1H), 7.69 (s, 1H), 7.63 (d, J=8.5 Hz, 1H), 7.39 (d, J=8.6 Hz, 1H), 6.69 (dd, J=7.7, 4.7 Hz, 1H), 6.57 (br s, 2H), 2.76 (tq, J=7.4, 3.8 Hz, 1H), 2.27 (s, 3H), 0.65-0.53 (m, 1H), 0.53-0.42 (m, 1H), 0.14-0.03 (m, 1H), 0.02-−0.12 (m, 1H).

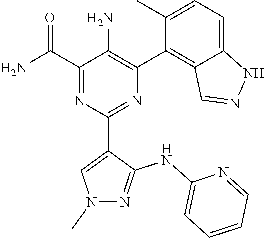 [SMILES: Cc1ccc2[nH]ncc2c1-c1nc(-c2cn(C)nc2Nc2ccccn2)nc(C(N)=O)c1N]

### Compound 916 (5-amino-6-(5-methyl-1H-indazol-4-yl)-2-(1-methyl-3-(pyridin-2-ylamino)-1H-pyrazol-4-yl)pyrimidine-4-carboxamide)

Step 1. A RBF was charged with 4-bromo-1-methyl-pyrazol-3-amine (500 mg, 2.84 mmol), potassium 2-ethylhexanoate (1.14 g, 6.25 mmol), bis(pinacolato)diboron (866 mg, 3.41 mmol), dicyclohexyl-[2-(2,4,6-triisopropylphenyl)phenyl]phosphane (68 mg, 142.0 μmol) isopropyl acetate (14 mL). The mixture was stirred under N₂ for 5 min at 40° C. then XPhos Pd(allyl)Cl (94 mg, 142.0 μmol) was added. The mixture was stirred at 40° C. for overnight. The reaction mixture was diluted with EtOAc and water. The aqueous layer was cut and the organic layer was washed with brine, dried over MgSO₄, filtered and concentrated. The residue was purified by silica gel chromatography eluting with a gradient of EtOAc (60 to 100%) in heptane to provide 1-methyl-4-(4,4,5,5-tetramethyl-1,3,2-dioxaborolan-2-yl)pyrazol-3-amine (220 mg, 35% yield). MS: [M+H]⁺: 224.1.

Step 2. A MW vessel was charged with Intermediate AB (225 mg, 541.0 μmol), 1-methyl-4-(4,4,5,5-tetramethyl-1,3,2-dioxaborolan-2-yl)pyrazol-3-amine (145 mg, 649.2 μmol, dioxane (3 mL) and 2 M aqueous solution of K₂CO₃ (676 μL, 1.352 mmol). Pd(dppf)Cl₂·DCM (22 mg, 27.1 μmol) was added and the vessel was flushed with N₂, sealed and stirred at 105° C. for 2 h. The cooled reaction mixture was diluted with EtOAc and water. The aqueous layer was cut and the organic layer was washed with brine, dried over MgSO₄, filtered and concentrated. The residue was purified by silica gel chromatography eluting with a gradient of EtOAc (50 to 100%) in heptane to provide ethyl 5-amino-2-(3-amino-1-methyl-pyrazol-4-yl)-6-(5-methyl-1-tetrahydropyran-2-yl-indazol-4-yl)pyrimidine-4-carboxylate (166 mg, 64% yield). MS: [M+H]⁺: 476.9.

Step 3. To a MW vial containing ethyl 5-amino-2-(3-amino-1-methyl-pyrazol-4-yl)-6-(5-methyl-1-tetrahydropyran-2-yl-indazol-4-yl)pyrimidine-4-carboxylate (77 mg, 161.6 μmol) and 2-bromopyridine (16 μL, 165.07 μmol) was added toluene (0.75 mL) followed by cesium carbonate (133 mg, 406.7 μmol), Xantphos (23 mg, 39.9 μmol) and Pd(OAc)₂ (6.0 mg, 26.8 μmol). The mixture was bubbled through with N₂, capped and stirred at 90° C. overnight. The cooled reaction mixture was adsorbed onto silica and the residue was purified by silica gel chromatography eluting with a gradient of MeOH (0 to 10%) in DCM to provide ethyl 5-amino-2-[1-methyl-3-(2-pyridylamino)pyrazol-4-yl]-6-(5-methyl-1-tetrahydropyran-2-yl-indazol-4-yl)pyrimidine-4-carboxylate (34 mg, 38% yield). MS: [M+H]⁺: 554.0.

Step 4. A MW vial was charged with ethyl 5-amino-2-[1-methyl-3-(2-pyridylamino)pyrazol-4-yl]-6-(5-methyl-1-tetrahydropyran-2-yl-indazol-4-yl)pyrimidine-4-carboxylate (34 mg, 61.4 μmol), MeOH (1 mL) and 7 N ammonia solution in MeOH (1 mL, 7 mmol). The vial was sealed and stirred at 85° C. for 6 h. The solvent was evaporated and the residue was dissolved in DCM (1.5 mL) and TFA (500 μL, 6.49 mmol). The mixture was aged at rt for 2 h. The solvent was evaporated and the crude was redissolved in DMSO, neutralized with Et₃N, filtered and the filtrate was purified by preparative HPLC C18 column eluting with ACN/water/0.1% formic acid to provide 5-amino-6-(5-methyl-1H-indazol-4-yl)-2-[1-methyl-3-(2-pyridylamino)pyrazol-4-yl]pyrimidine-4-carboxamide (6 mg, 22% yield). MS: [M+H]⁺: 441.0; ¹H NMR (400 MHz, DMSO-d6) δ 13.16 (s, 1H), 8.55 (s, 1H), 8.50 (s, 2H), 8.03 (d, J=4.9 Hz, 1H), 7.94 (d, J=2.7 Hz, 1H), 7.74 (s, 2H), 7.67 (s, 1H), 7.62 (d, J=8.6 Hz, 1H), 7.41 (d, J=8.6 Hz, 1H), 6.82 (s, 2H), 6.26 (s, 1H), 3.85 (s, 3H), 2.28 (s, 3H)

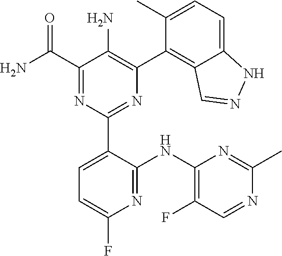 [SMILES: Cc1ncc(F)c(Nc2nc(F)ccc2-c2nc(C(N)=O)c(N)c(-c3c(C)ccc4[nH]ncc34)n2)n1]

### Compound 954 (5-amino-2-(6-fluoro-2-((5-fluoro-2-methylpyrimidin-4-yl)amino)pyridin-3-yl)-6-(5-methyl-1H-indazol-4-yl)pyrimidine-4-carboxamide)

Step 1. A pressure vessel was charged with Intermediate AQ (1.14 g, 5.97 mmol), bis(pinacoloto)diboron (1.82 g, 7.16 mmol) and potassium acetate (1.57 g, 16.0 mmol). Dioxane (30 mL) was added and the vessel was flushed with N₂. Pd(dppf)Cl₂·DCM (196 mg, 240.0 μmol) was added and the vessel was flushed again with N₂, sealed and stirred at 110° C. for 2 h. Intermediate AB (2.0 g, 4.81 mmol) was added to the cooled reaction mixture followed by 2 M aqueous solution of K₂CO₃ (6.0 mL, 12.0 mmol) and more Pd(dppf)Cl₂·DCM (196 mg, 240.0 μmol). The vessel was flushed with N₂, sealed and stirred at 110° C. for 1 h. The cooled reaction mixture was diluted with water and EtOAc and filtered through a Celite™. The layers were separated and the aqueous layer was extracted with EtOAc (2×). The combined organic extracts was washed with brine, dried over Na₂SO₄, filtered and adsorbed onto silica. The residue was purified by silica gel chromatography eluting with a gradient of EtOAc (0 to 100%) in heptane to provide ethyl 5-amino-2-(2-amino-6-fluoro-3-pyridyl)-6-(5-methyl-1-tetrahydropyran-2-yl-indazol-4-yl)pyrimidine-4-carboxylate (2.32 g, 98% yield). MS: [M+H]⁺: 491.9.

Step 2. A MW vessel was charged with ethyl 5-amino-2-(2-amino-6-fluoro-3-pyridyl)-6-(5-methyl-1-tetrahydropyran-2-yl-indazol-4-yl)pyrimidine-4-carboxylate (500 mg, 1.02 mmol), 4-chloro-5-fluoro-2-methyl-pyrimidine (179 mg, 1.22 mmol), Pd(OAc)₂ (11 mg, 49.0 μmol), rac-BINAP (60 mg, 96.4 μmol) and toluene (7.5 mL). K₃PO₄ (546 mg, 2.57 mmol) was added, the vessel was flushed with N₂, sealed and stirred at 110° C. for 3 h. The cooled reaction mixture was diluted with EtOAc and water. The aqueous layer was cut and extracted with EtOAc. The combined organic layer was washed with brine, dried over MgSO₄, filtered and concentrated. The residue was purified by silica gel chromatography eluting with a gradient of EtOAc (30 to 70%) in heptane to provide ethyl 5-amino-2-[6-fluoro-2-[(5-fluoro-2-methyl-pyrimidin-4-yl)amino]-3-pyridyl]-6-(5-methyl-1-tetrahydropyran-2-yl-indazol-4-yl)pyrimidine-4-carboxylate (503 mg, 82% yield). MS: [M+H]⁺: 602.4.

Step 3. A MW vial was charged with ethyl 5-amino-2-[6-fluoro-2-[(5-fluoro-2-methyl-pyrimidin-4-yl)amino]-3-pyridyl]-6-(5-methyl-1-tetrahydropyran-2-yl-indazol-4-yl)pyrimidine-4-carboxylate (2.0 g, 3.32 mmol). 7 N ammonia solution in MeOH (10 mL, 70 mmol) was added to the mixture and the vial was sealed and stirred at 85° C. for 5 h then allowed to cool to rt overnight. The solvent was evaporated to give 5-amino-2-[6-fluoro-2-[(5-fluoro-2-methyl-pyrimidin-4-yl)amino]-3-pyridyl]-6-(5-methyl-1-tetrahydropyran-2-yl-indazol-4-yl)pyrimidine-4-carboxamide (1.88 g, 99% yield). MS: [M+H]⁺: 573.3.

Step 4. A RBF was charged with 5-amino-2-[6-fluoro-2-[(5-fluoro-2-methyl-pyrimidin-4-yl)amino]-3-pyridyl]-6-(5-methyl-1-tetrahydropyran-2-yl-indazol-4-yl)pyrimidine-4-carboxamide (2.50 g, 4.37 mmol), DCM (10 mL) and triethylsilane (2.1 mL, 13.15 mmol). TFA (10 mL, 129.80 mmol) was added portionwise and the mixture was stirred at rt for 2 h. The solvent was evaporated and the crude mixture was diluted with a minimum of MeOH then added slowly to a stirring aqueous solution of 5% wt NaHCO₃. The mixture was sonicated then stirred overnight at rt. The solid was filtered and the cake was rinsed with heptane. The solid was triturated in boiling acetone to give 5-amino-2-(6-fluoro-2-((5-fluoro-2-methylpyrimidin-4-yl)amino)pyridin-3-yl)-6-(5-methyl-1H-indazol-4-yl)pyrimidine-4-carboxamide (1.76 g, 82% yield). MS: [M+H]⁺: 489.3; ¹H NMR (DMSO-d₆) δ 13.14 (s, 1H), 11.77 (s, 1H), 9.19 (t, J=8.5 Hz, 1H), 8.70 (s, 1H), 8.31 (d, J=3.3 Hz, 1H), 7.99 (s, 1H), 7.67 (s, 1H), 7.62 (d, J=8.6 Hz, 1H), 7.38 (d, J=8.6 Hz, 1H), 6.90 (dd, J=8.5, 3.3 Hz, 1H), 6.68 (s, 2H), 2.37 (s, 3H), 2.24 (s, 3H).

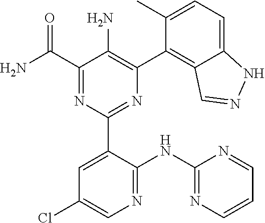 [SMILES: Cc1ccc2[nH]ncc2c1-c1nc(-c2cc(Cl)cnc2Nc2ncccn2)nc(C(N)=O)c1N]

### Compound 955 (5-amino-2-(2-chloro-5-(pyrimidin-2-ylamino)pyridin-4-yl)-6-(5-methyl-1H-indazol-4-yl)pyrimidine-4-carboxamide)

Step 1. A MW vessel was charged with a 0.3 M solution of Intermediate AL in DMF (2 mL, 0.6 mmol), 6-chloro-4-iodo-pyridin-3-amine (197.6 mg, 776.4 μmol) and DMF (0.6 mL). Copper(I) iodide (25.7 mg, 135.0 μmol) and lithium chloride (65.7 mg, 1.55 mmol) were added followed by bis(tri-tert-butylphosphine)palladium(0) (31.4 mg, 61.5 μmol). The vessel was flushed with N₂, capped and stirred at 120° C. for 1 h. The cooled reaction mixture was filtered over Celite™ and concentrated. The residue was purified by silica gel chromatography eluting with a gradient of EtOAc (30 to 70%) in heptane to provide ethyl 5-amino-2-(5-amino-2-chloro-4-pyridyl)-6-(5-methyl-1-tetrahydropyran-2-yl-indazol-4-yl)pyrimidine-4-carboxylate (121 mg, 40% yield). MS: [M+H]⁺: 508.1/510.1.

Step 2. A pear-shaped flask containing ethyl 5-amino-2-(5-amino-2-chloro-4-pyridyl)-6-(5-methyl-1-tetrahydropyran-2-yl-indazol-4-yl)pyrimidine-4-carboxylate (121 mg, 238.2 μmol) was charged with 2-bromopyrimidine (42 mg, 262.0 μmol), Pd(OAc)₂ (5.4 mg, 23.8 μmol), rac-BINAP (14.8 mg, 23.8 μmol), cesium carbonate (194 mg, 595.5 μmol) and toluene (2 mL). The flask was fitted with a condenser and the mixture was stirred at reflux overnight under N₂. The solvent was evaporated and the residue was dissolved in DCM/MeOH. The reaction mixture was acidified with 10% aqueous HCl solution then treated with 2 M (trimethylsilyl)diazomethane solution in hexanes (1 mL, 1 mmol). The mixture was stirred for 5 min at rt then washed with water and brine consecutively, dried over MgSO₄, filtered and concentrated.

The residue was purified by silica gel chromatography eluting with a gradient of EtOAc (50 to 100%) in heptane to provide methyl 5-amino-2-[2-chloro-5-(pyrimidin-2-ylamino)-4-pyridyl]-6-(5-methyl-1-tetrahydropyran-2-yl-indazol-4-yl)pyrimidine-4-carboxylate (88 mg, 65% yield). MS: [M+H]⁺: 572.1/574.1.

Step 3. A MW vial was charged with methyl 5-amino-2-[2-chloro-5-(pyrimidin-2-ylamino)-4-pyridyl]-6-(5-methyl-1-tetrahydropyran-2-yl-indazol-4-yl)pyrimidine-4-carboxylate (88 mg, 153.8 μmol), MeOH (1 mL) and 7 N ammonia solution in MeOH (1 mL, 7 mmol). The vial was sealed and stirred at 85° C. for 1.5 h. The solvent was evaporated and the residue was dissolved in DCM (1.5 mL) and TFA (0.5 mL). The mixture was aged at rt for 1.5 h. The solvent was evaporated and the residue was redissolved in DMSO, neutralized with Et₃N, filtered and the filtrate was purified by preparative HPLC eluting with a gradient of acetonitrile in water containing 10 mM ammonium bicarbonate (pH adjusted to 10 with NH₄OH) to provide 5-amino-2-[2-chloro-5-(pyrimidin-2-ylamino)-4-pyridyl]-6-(5-methyl-1H-indazol-4-yl)pyrimidine-4-carboxamide (24 mg, 33% yield). MS: [M+H]⁺: 473.1; ¹H NMR (DMSO-d₆) δ 13.17 (s, 1H), 11.63 (s, 1H), 9.57 (s, 1H), 8.71 (s, 1H), 8.54 (s, 1H), 8.43 (d, J=4.8 Hz, 2H), 8.05 (s, 1H), 7.72 (s, 1H), 7.64 (d, J=8.5 Hz, 1H), 7.41 (d, J=8.6 Hz, 1H), 7.00-6.79 (m, 3H), 2.28 (s, 3H).

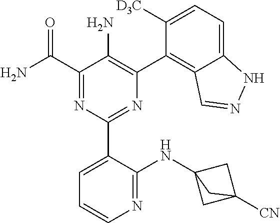 [SMILES: [2H]C([2H])([2H])c1ccc2[nH]ncc2c1-c1nc(-c2cccnc2NC23CC(C#N)(C2)C3)nc(C(N)=O)c1N]

### Compound 957 (5-amino-2-(6-fluoro-2-((5-fluoro-2-methylpyrimidin-4-yl)amino)pyridin-3-yl)-6-(5-methyl-1H-indazol-4-yl)pyrimidine-4-carboxamide)

Step 1. To a solution of Intermediate CM (115 mg, 435.4 μmol) in dioxane (10 mL) were added potassium acetate (106 mg, 1.08 mmol), Pd(dppf)Cl₂ (19 mg, 26.0 μmol) and bis(pinacolato)diboron (138 mg, 543.4 μmol). The mixture was degassed in vacuo, back-filled with N₂ and stirred at 110° C. under N₂ for 1 h. Intermediate CA (150 mg, 358.1 μmol), Pd(dppf)Cl₂ (19 mg, 26.0 μmol) and 2 M aqueous solution of K₂CO₃ (660 μL, 1.32 mmol) were added to the mixture and stirring was continued for 1 h at 110° C. The cooled reaction mixture was diluted with water and extracted with EtOAc (3×40 mL). The organic extracts were washed with bine, dried over sodium sulfate, filtered and concentrated. The residue was purified by silica gel chromatography eluting with a gradient of EtOAc (20 to 60%) in heptane to provide ethyl 5-amino-2-[2-[(3-cyano-1-bicyclo[1.1.1]pentanyl)amino]-3-pyridyl]-6-[1-tetrahydropyran-2-yl-5-(trideuteriomethyl)indazol-4-yl]pyrimidine-4-carboxylate (170 mg, 84% yield).

Step 2. The mixture of ethyl 5-amino-2-[2-[(3-cyano-1-bicyclo[1.1.1]pentanyl)amino]-3-pyridyl]-6-[1-tetrahydropyran-2-yl-5-(trideuteriomethyl)indazol-4-yl]pyrimidine-4-carboxylate (50 mg, 88.1 μmol) and 7 N solution of ammonia in MeOH (2.5 mL, 17.5 mmol) in a sealed vial was stirred at 85° C. for 20 min. The volatiles were removed in vacuo and the solid was dissolved in MeOH (1 mL) and 4 M HCl solution in dioxane (400 μL, 1.6 mmol). The mixture was stirred at rt for 2 h. The volatiles were removed in vacuo and the residue was dissolved in MeOH, basified with Et₃N and concentrated to dryness. The residue was purified by preparative HPLC C18 column eluting with ACN/water/0.1% formic acid to provide 5-amino-2-[2-[(3-cyano-1-bicyclo[1.1.1]pentanyl)amino]-3-pyridyl]-6-[5-(trideuteriomethyl)-1H-indazol-4-yl]pyrimidine-4-carboxamide (15 mg, 37% yield). MS: [M+H]⁺: 455.2; ¹H NMR (DMSO-d₆) δ 13.18 (s, 1H), 9.58 (s, 1H), 8.83 (d, J=7.8 Hz, 1H), 8.68-8.42 (m, 1H), 8.09 (d, J=4.8 Hz, 1H), 7.90 (s, 1H), 7.73 (s, 1H), 7.61 (d, J=8.6 Hz, 1H), 7.35 (d, J=8.5 Hz, 1H), 6.73 (m, 1H), 6.69-6.49 (m, 2H), 2.34 (s, 6H).

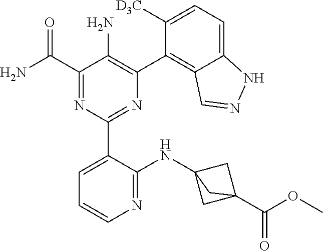 [SMILES: [2H]C([2H])([2H])c1ccc2[nH]ncc2c1-c1nc(-c2cccnc2NC23CC(C(=O)OC)(C2)C3)nc(C(N)=O)c1N]

### Compound 961 (methyl 3-((3-(5-amino-4-carbamoyl-6-(5-(methyl-d3)-1H-indazol-4-yl)pyrimidin-2-yl)pyridin-2-yl)amino)bicyclo[1.1.1]pentane-1-carboxylate)

Methyl 3-((3-(5-amino-4-carbamoyl-6-(5-(methyl-d3)-1H-indazol-4-yl)pyrimidin-2-yl)pyridin-2-yl)amino)bicyclo[1.1.1]pentane-1-carboxylate (11 mg, 26% yield) was also recovered during the preparative HPLC purification of compound 957. MS: [M+H]⁺: 488.1; ¹H NMR (DMSO-d₆) δ 13.18 (s, 1H), 9.53 (s, 1H), 8.83 (d, J=7.7 Hz, 1H), 8.57 (d, J=2.3 Hz, 1H), 8.09 (d, J=4.7 Hz, 1H), 7.90 (d, J=2.3 Hz, 1H), 7.73 (s, 1H), 7.60 (d, J=8.6 Hz, 1H), 7.35 (d, J=8.5 Hz, 1H), 6.69 (m, 1H), 6.64 (s, 2H), 3.55 (s, 3H), 2.20-2.05 (m, 6H).

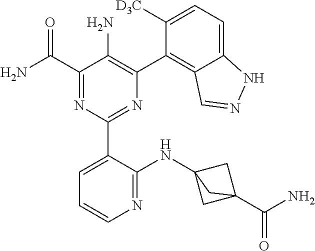 [SMILES: [2H]C([2H])([2H])c1ccc2[nH]ncc2c1-c1nc(-c2cccnc2NC23CC(C(N)=O)(C2)C3)nc(C(N)=O)c1N]

### Compound 962 (5-amino-2-(2-((3-carbamoylbicyclo[1.1.1]pentan-1-yl)amino)pyridin-3-yl)-6-(5-(methyl-d3)-1H-indazol-4-yl)pyrimidine-4-carboxamide)

5-amino-2-(2-((3-carbamoylbicyclo[1.1.1]pentan-1-yl)amino)pyridin-3-yl)-6-(5-(methyl-d3)-1H-indazol-4-yl)pyrimidine-4-carboxamide (3 mg, 7% yield) was also recovered during the preparative HPLC purification of compound 957. MS: [M+H]⁺: 472.2; ¹H NMR (DMSO-d₆) δ 13.18 (s, 1H), 9.51 (s, 1H), 8.84 (dd, J=7.8, 1.9 Hz, 1H), 8.58 (s, 1H), 8.42 (s, 1H), 8.09 (dd, J=4.8, 1.9 Hz, 1H), 7.91 (s, 1H), 7.72 (s, 1H), 7.60 (dd, J=8.6, 1.0 Hz, 1H), 7.35 (d, J=8.6 Hz, 1H), 6.72 (dd, J=7.7, 4.8 Hz, 1H), 6.60 (s, 2H), 2.23 (s, 6H).

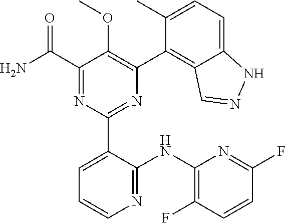 [SMILES: COc1c(C(N)=O)nc(-c2cccnc2Nc2nc(F)ccc2F)nc1-c1c(C)ccc2[nH]ncc12]

### Compound 963 (2-(2-((3,6-difluoropyridin-2-yl)amino)pyridin-3-yl)-5-methoxy-6-(5-methyl-1H-indazol-4-yl)pyrimidine-4-carboxamide)

Step 1. A mixture of methyl 2,6-dichloro-5-methoxy-pyrimidine-4-carboxylate (2.5 g, 8.96 mmol), 5-methyl-1-tetrahydropyran-2-yl-4-(4,4,5,5-tetramethyl-1,3,2-dioxaborolan-2-yl)indazole (3.2 g, 9.35 mmol), 2 M aqueous solution of K₃PO₄ (7.5 mL, 15 mmol) and Pd(dtbpf)Cl₂ (590 mg, 905.3 μmol) in dioxane (50 mL) was bubbled through with N₂ then heated at 80° C. for 1 h. The cooled reaction mixture was diluted with water and EtOAc and was filtered over Celite™. The layers were portioned and the aq layer was extracted twice with EtOAc. The combined organic layers was washed with brine, dried over Na₂SO₄, filtered and concentrated. The residue was purified by silica gel chromatography eluting with a gradient of EtOAc (0 to 75%) in heptane to provide methyl 2-chloro-5-methoxy-6-(5-methyl-1-tetrahydropyran-2-yl-indazol-4-yl)pyrimidine-4-carboxylate (910 mg, 24% yield). MS: [M+H]⁺: 417.3.

Step 2. A RBF was loaded with methyl 2-chloro-5-methoxy-6-(5-methyl-1-tetrahydropyran-2-yl-indazol-4-yl)pyrimidine-4-carboxylate (871 mg, 2.09 mmol), 3-(4,4,5,5-tetramethyl-1,3,2-dioxaborolan-2-yl)pyridin-2-amine (700 mg, 3.18 mmol), dioxane (10 mL) and 2 M aqueous solution of K₂CO₃ (2.75 mL, 5.5 mmol). The mixture was bubbled through with N₂ and Pd(dtbpf)Cl₂ (150 mg, 230.2 μmol) was added. The mixture was bubbled through again with N₂ and stirred at 110° C. for 1 h. The reaction mixture was cooled to rt, poured into water and extracted with EtOAc (3×). The combined organic layers was washed with brine, dried over Na₂SO₄, filtered and concentrated. The residue was purified by silica gel chromatography eluting with a gradient of EtOAc (0 to 100%) in heptane to provide methyl 2-(2-amino-3-pyridyl)-5-methoxy-6-(5-methyl-1-tetrahydropyran-2-yl-indazol-4-yl)pyrimidine-4-carboxylate (450 mg, 45% yield). MS: [M+H]⁺: 475.3.

Step 3. To a solution of methyl 2-(2-amino-3-pyridyl)-5-methoxy-6-(5-methyl-1-tetrahydropyran-2-yl-indazol-4-yl)pyrimidine-4-carboxylate (450 mg, 948.4 μmol) in toluene (5 mL) were added 2-bromo-3,6-difluoro-pyridine (260 mg, 1.34 mmol), Pd(OAc)₂ (35 mg, 155.9 μmol), cesium carbonate (785 mg, 2.41 mmol) and Xantphos (140 mg, 242.0 μmol). The mixture was degassed in vacuo, back-filled with N₂ and stirred at 110° C. for 1 h. The cooled reaction mixture was diluted with DCM and filtrated on Celite™. The volatiles were removed in vacuo. The residue was purified by silica gel chromatography eluting with a gradient of EtOAc (20 to 100%) in heptane to provide methyl 2-[2-[(3,6-difluoro-2-pyridyl)amino]-3-pyridyl]-5-methoxy-6-(5-methyl-1-tetrahydropyran-2-yl-indazol-4-yl)pyrimidine-4-carboxylate (460 mg, 83% yield). MS: [M+H]⁺: 588.3.

Step 4. 7 N ammonia solution in MeOH (2 mL, 14 mmol) was added to methyl 2-[2-[(3,6-difluoro-2-pyridyl)amino]-3-pyridyl]-5-methoxy-6-(5-methyl-1-tetrahydropyran-2-yl-indazol-4-yl)pyrimidine-4-carboxylate (400 mg, 680.76 μmol) and the sealed microwave vial was stirred at 80° C. for 1 h then evaporated to dryness. The residue was dissolved in 4 M HCl solution in dioxane (2.0 mL, 8 mmol) and MeOH (2 mL) and the solution was stirred at 50° C. for 30 min in a sealed microwave vial. The volatiles were evaporated to dryness and the residue was taken into 1 mL of DCM. A few drops of Et₃N were added until pH was slightly basic and the mixture was evaporated to dryness. The residue was purified by preparative HPLC C18 column eluting with ACN/water/0.1% formic acid to provide 2-[2-[(3,6-difluoro-2-pyridyl)amino]-3-pyridyl]-5-methoxy-6-(5-methyl-1H-indazol-4-yl)pyrimidine-4-carboxamide (260 mg, 78% yield). MS: [M+H]⁺: 489.3; ¹H NMR (DMSO-d₆) δ 13.17 (s, 1H), 11.28 (s, 1H), 8.72 (dd, J=7.9, 2.0 Hz, 1H), 8.45-8.25 (m, 2H), 8.05 (s, 1H), 7.80 (td, J=8.9, 6.3 Hz, 1H), 7.74 (s, 1H), 7.60 (d, J=8.5 Hz, 1H), 7.36 (d, J=8.6 Hz, 1H), 7.09 (dd, J=7.9, 4.7 Hz, 1H), 6.81 (dt, J=8.4, 2.8 Hz, 1H), 3.41 (s, 3H), 2.29 (s, 3H).

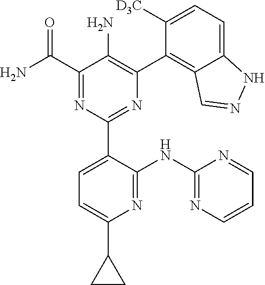 [SMILES: [2H]C([2H])([2H])c1ccc2[nH]ncc2c1-c1nc(-c2ccc(C3CC3)nc2Nc2ncccn2)nc(C(N)=O)c1N]

### Compound 974 (5-amino-2-(6-cyclopropyl-2-(pyrimidin-2-ylamino)pyridin-3-yl)-6-(5-(methyl-d3)-1H-indazol-4-yl)pyrimidine-4-carboxamide)

Step 1. To a solution of Intermediate CE (306 mg, 1.44 mmol) in dioxane (10 mL) were added potassium acetate (35 mg, 3.60 mmol), Pd(dppf)Cl₂ (69 mg, 94.0 μmol) and bis(pinacolato)diboron (463 mg, 1.82 mmol). The mixture was degassed in vacuo, back-filled with N₂ and stirred at 110° C. under N₂ for 1 h. Intermediate CA (500 mg, 1.19 mmol), Pd(dppf)Cl₂ (69 mg, 94.0 μmol) and 2 M aqueous solution of potassium carbonate (2.12 mL, 4.24 mmol) were added to the cooled reaction mixture and stirring was continued for 1 h 110° C. The cooled reaction mixture was diluted with water and extracted with EtOAc (3×40 mL). The combined organic extracts was washed with bine, dried over sodium sulfate, filtered and concentrated. The residue was purified by silica gel chromatography eluting with a gradient of EtOAc (20 to 60%) in heptane to provide ethyl 5-amino-2-(2-amino-6-cyclopropyl-3-pyridyl)-6-[1-tetrahydropyran-2-yl-5-(trideuteriomethyl)indazol-4-yl]pyrimidine-4-carboxylate (400 mg, 65% yield). MS: [M+H]⁺: 517.2.

Step 2. To a solution of ethyl 5-amino-2-(2-amino-6-cyclopropyl-3-pyridyl)-6-[1-tetrahydropyran-2-yl-5-(trideuteriomethyl)indazol-4-yl]pyrimidine-4-carboxylate (100 mg, 193.6 μmol) in toluene (2 mL) were added 2-bromopyrimidine (57 mg, 358.5 μmol), Pd(OAc)₂ (5 mg, 22.3 μmol), cesium carbonate (189 mg, 580.1 μmol) and rac-BINAP (24 mg, 38.5 μmol). The mixture was degassed in vacuo, back-filled with N₂ and stirred at 110° C. for 2 h. The volatiles were removed in vacuo and the residue was purified by silica gel chromatography eluting with a gradient of EtOAc (40 to 100%) in heptane to provide ethyl 5-amino-2-[6-cyclopropyl-2-(pyrimidin-2-ylamino)-3-pyridyl]-6-[1-tetrahydropyran-2-yl-5-(trideuteriomethyl)indazol-4-yl]pyrimidine-4-carboxylate (35 mg, 30% yield). MS: [M+H]⁺: 595.3.

Step 3. A mixture of ethyl 5-amino-2-[6-cyclopropyl-2-(pyrimidin-2-ylamino)-3-pyridyl]-6-[1-tetrahydropyran-2-yl-5-(trideuteriomethyl)indazol-4-yl]pyrimidine-4-carboxylate (35 mg, 58.9 μmol) and 7 N ammonia solution in MeOH (2 mL, 14 mmol) in a sealed vial was stirred at 85° C. for 20 min. The volatiles were removed in vacuo and the solid was dissolved in MeOH (1 mL) and 4 M HCl solution in dioxane (300 μL, 1.2 mmol). The mixture was stirred at rt for 20 min. The volatiles were removed in vacuo. The residue was dissolved in MeOH, basified with Et₃N, and concentrated to dryness. The residue was purified by preparative HPLC C18 column eluting with ACN/water/0.1% formic acid to provide 5-amino-2-[6-cyclopropyl-2-(pyrimidin-2-ylamino)-3-pyridyl]-6-[5-(trideuteriomethyl)-1H-indazol-4-yl]pyrimidine-4-carboxamide (15 mg, 53% yield). MS: [M+H]⁺: 482.1; ¹H NMR (DMSO-d6) δ 13.11 (s, 1H), 11.09 (s, 1H), 8.56 (d, J=8.0 Hz, 1H), 8.38 (s, 1H), 8.31 (d, J=4.8 Hz, 2H), 8.00-7.85 (m, 1H), 7.66-7.53 (m, 2H), 7.35 (d, J=8.4 Hz, 1H), 7.00 (d, J=8.1 Hz, 1H), 6.81 (t, J=4.8 Hz, 1H), 6.45 (s, 2H), 2.02 (td, J=7.9, 4.0 Hz, 1H), 1.04-0.91 (m, 2H), 0.91-0.74 (m, 2H).

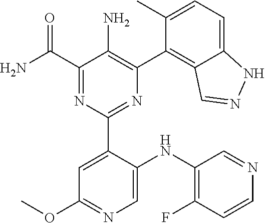 [SMILES: COc1cc(-c2nc(C(N)=O)c(N)c(-c3c(C)ccc4[nH]ncc34)n2)c(Nc2cnccc2F)cn1]

### Compound 976 (5-amino-2-(5-((3-fluoropyridin-2-yl)amino)-2-methoxypyridin-4-yl)-6-(5-methyl-1H-indazol-4-yl)pyrimidine-4-carboxamide)

Step 1. 2,3-Difluoropyridine (353 mg, 2.94 mmol) and 2-fluoro-5-iodo-4-methylpyridine (200 mg, 736 μmol) were dissolved in DMF (3.7 mL). Sodium hydride (60% dispersion in mineral oil, 147 mg, 3.68 mmol) was added portion wise at rt and the mixture was stirred at rt for 1 h. MeOH (1 mL) and silica were added and the mixture was concentrated. The residue was purified by silica gel chromatography eluting with a gradient of EtOAc (5 to 100%) in heptane to provide N-(4-bromo-6-fluoropyridin-3-yl)-3-fluoropyridin-2-amine (160 mg, 76% yield). MS: [M+H]⁺: 286.9.

Step 2. In a vial, N-(4-bromo-6-fluoropyridin-3-yl)-3-fluoropyridin-2-amine (160 mg, 561 μmol), lithium chloride (40.0 mg, 935 μmol), copper(I) iodide (14.2 mg, 74.8 μmol), Intermediate AL (250 mg, 374 μmol), were dissolved in DMF (2.50 mL). The mixture was degassed with nitrogen for 10 minutes and bis(tri-t-butylphosphine)palladium(0) (19.1 mg, 36.7 μmol) was added. The vial was sealed and heated to 90° C. for 12 hours with stirring. The reaction was adsorbed directly onto silica gel. The residue was purified by silica gel chromatography eluting with a gradient of EtOAc (5 to 100%) in heptane to provide ethyl 5-amino-2-(2-fluoro-5-((3-fluoropyridin-2-yl)amino)pyridin-4-yl)-6-(5-methyl-1-(tetrahydro-2H-pyran-2-yl)-1H-indazol-4-yl)pyrimidine-4-carboxylate (120 mg, 27% yield). MS: [M+H]⁺: 587.3.

Step 3. In a sealed tube, ethyl 5-amino-2-(2-fluoro-5-((3-fluoropyridin-2-yl)amino)pyridin-4-yl)-6-(5-methyl-1-(tetrahydro-2H-pyran-2-yl)-1H-indazol-4-yl)pyrimidine-4-carboxylate (120 mg, 205 μmol) was dissolved in MeOH (1.0 mL) and 7 N ammonia solution in MeOH (292 μL, 2.04 mmol) was added. The mixture was heated at 80° C. for 12 h. The cooled reaction mixture was concentrated and the residue was dissolved in MeOH (1.0 mL) and 4 M HCl solution in dioxane (510 μL, 2.04 mmol). The solution was stirred at 50° C. for 2 h with stirring. The mixture was concentrated and the residue was purified by preparative HPLC eluting with a gradient of acetonitrile in water containing 10 mM ammonium bicarbonate (pH adjusted to 10 with NH₄OH) to provide 5-amino-2-(5-((3-fluoropyridin-2-yl)amino)-2-methoxypyridin-4-yl)-6-(5-methyl-1H-indazol-4-yl)pyrimidine-4-carboxamide (7.5 mg, 8% yield). MS: [M+H]⁺: 486.2; ¹H NMR (DMSO-d₆) δ 13.13 (s, 1H), 10.93 (s, 1H), 9.35 (s, 1H), 8.67 (s, 1H), 7.96 (s, 1H), 7.93 (s, 1H), 7.88 (d, J=4.8 Hz, 1H), 7.66-7.57 (m, 2H), 7.38 (d, J=8.6 Hz, 1H), 7.29 (s, 1H), 6.75-6.65 (m, 2H), 3.88 (s, 3H), 2.20 (s, 3H).

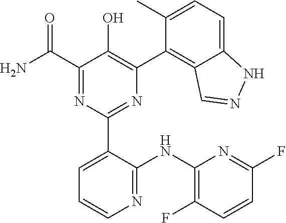 [SMILES: Cc1ccc2[nH]ncc2c1-c1nc(-c2cccnc2Nc2nc(F)ccc2F)nc(C(N)=O)c1O]

### Compound 806 (2-(2-((3,6-difluoropyridin-2-yl)amino)pyridin-3-yl)-5-hydroxy-6-(5-methyl-1H-indazol-4-yl)pyrimidine-4-carboxamide)

1 M solution of boron tribromide in DCM (1 mL, 1 mmol) was added to a mixture of Compound 963 (150 mg, 307.1 μmol) in DCM (2 mL) and the mixture was stirred at rt for 2 h. The volatiles were evaporated to dryness. The solid was taken in 5 mL of DCM, 1 mL of MeOH was added and the mixture was evaporated to dryness again. The solid was taken in 5 mL of DCM, 1 mL of Et₃N was added and the mixture was evaporated to dryness. The residue was purified by preparative HPLC C18 column eluting with ACN/water/0.1% formic acid to provide 2-[2-[(3,6-difluoro-2-pyridyl)amino]-3-pyridyl]-5-hydroxy-6-(5-methyl-1H-indazol-4-yl)pyrimidine-4-carboxamide (45 mg, 30% yield). MS: [M+H]⁺: 475.3; ¹H NMR (DMSO-d₆) δ 13.06 (s, 2H), 11.19 (s, 1H), 9.25 (s, 1H), 9.10 (dd, J=7.8, 1.9 Hz, 1H), 8.73 (s, 1H), 8.29 (dd, J=4.7, 1.9 Hz, 1H), 7.75 (s, 1H), 7.70 (td, J=9.0, 6.5 Hz, 1H), 7.57 (dd, J=8.4, 1.0 Hz, 1H), 7.33 (d, J=8.6 Hz, 1H), 7.10 (dd, J=7.9, 4.7 Hz, 1H), 6.72 (ddd, J=8.5, 3.5, 2.3 Hz, 1H), 2.29 (s, 3H).

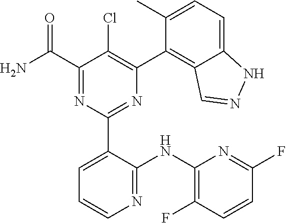 [SMILES: Cc1ccc2[nH]ncc2c1-c1nc(-c2cccnc2Nc2nc(F)ccc2F)nc(C(N)=O)c1Cl]

### Compound 314 (2-(2-((3,6-difluoropyridin-2-yl)amino)pyridin-3-yl)-5-hydroxy-6-(5-methyl-1H-indazol-4-yl)pyrimidine-4-carboxamide)

Step 1. A mixture of methyl 2,5,6-trichloropyrimidine-4-carboxylate (1.05 g, 4.35 mmol), 5-methyl-1-tetrahydropyran-2-yl-4-(4,4,5,5-tetramethyl-1,3,2-dioxaborolan-2-yl)indazole (1.57 g, 4.59 mmol), 2 M aqueous solution of K₃PO₄ (3.48 mL, 6.96 mmol) and Pd(dtbpf)Cl₂ (282 mg, 432.7 μmol) in dioxane (10 mL) was bubbled through with N₂, then heated at 85° C. overnight. The cooled reaction mixture was diluted with water and DCM and the mixture was filtrated over Celite™. The organic layer was separated and the aqueous layer was extracted with DCM twice. The combined organic layers was dried over Na₂SO₄, filtered and concentrated. The residue was purified by silica gel chromatography eluting with a gradient of EtOAc (20 to 100%) in heptane to provide methyl 2,5-dichloro-6-(5-methyl-1-tetrahydropyran-2-yl-indazol-4-yl)pyrimidine-4-carboxylate (286 mg, 16% yield). MS: [M+H]⁺: 421.3./423.3.

Step 2. A MW vial was loaded with methyl 2,5-dichloro-6-(5-methyl-1-tetrahydropyran-2-yl-indazol-4-yl)pyrimidine-4-carboxylate (128 mg, 303.8 μmol), 3-(4,4,5,5-tetramethyl-1,3,2-dioxaborolan-2-yl)pyridin-2-amine (86 mg, 390.8 μmol) and Pd(dtbpf)Cl₂ (20 mg, 30.7 μmol) in dioxane (2 mL) and 2 M aqueous K₂CO₃ solution of potassium carbonate (380 μL, 0.76 mmol). The mixture was bubbled through with N₂, capped and stirred at 110° C. for 35 min. The cooled reaction mixture was diluted with DCM and water. The layers were separated and the aqueous layer was extracted with DCM (2×). The combined organic extracts was dried over Na₂SO₄, filtered and adsorbed on silica. The residue was purified by silica gel chromatography eluting with a gradient of EtOAc (20 to 100%) in heptane to provide methyl 2-(2-amino-3-pyridyl)-5-chloro-6-(5-methyl-1-tetrahydropyran-2-yl-indazol-4-yl)pyrimidine-4-carboxylate (80 mg, 55% yield). MS: [M+H]⁺: 479.2.

Step 3. To a vial containing methyl 2-(2-amino-3-pyridyl)-5-chloro-6-(5-methyl-1-tetrahydropyran-2-yl-indazol-4-yl)pyrimidine-4-carboxylate (80 mg, 167.0 μmol), 2-bromo-3,6-difluoro-pyridine (60 mg, 309.3 μmol), Pd(OAc)₂ (5 mg, 22.3 μmol), Xantphos (25 mg, 43.2 μmol) and cesium carbonate (138 mg, 423.6 μmol) was added toluene (3 mL). N₂ was bubbled through the solution, the vial was capped and stirred at 110° C. for 1 h. The cooled reaction mixture was adsorbed onto silica using DCM and the residue was purified by silica gel chromatography eluting with a gradient of EtOAc (40 to 100%) in heptane to provide methyl 5-chloro-2-[2-[(3,6-difluoro-2-pyridyl)amino]-3-pyridyl]-6-(5-methyl-1-tetrahydropyran-2-yl-indazol-4-yl)pyrimidine-4-carboxylate (98 mg, 99% yield). MS: [M+H]⁺: 592.2.

Step 4. A MW vial containing methyl 5-chloro-2-[2-[(3,6-difluoro-2-pyridyl)amino]-3-pyridyl]-6-(5-methyl-1-tetrahydropyran-2-yl-indazol-4-yl)pyrimidine-4-carboxylate (107 mg, 180.7 μmol) in 7 N ammonia solution in MeOH (3 mL, 21 mmol) was capped and stirred at 80° C. for 20 min. The solution was cooled to rt, concentrated and the resulting solid was dissolved in a mixture of MeOH (3 mL) and 4 M HCl solution in dioxane (1.5 mL, 6 mmol) then stirred at 45° C. for 20 min before being concentrated to dryness. The residue was dissolved in MeOH, basicified with Et₃N and concentrated again. The residue was purified by preparative HPLC C18 column eluting with ACN/water/0.1% formic acid to provide 5-chloro-2-[2-[(3,6-difluoro-2-pyridyl)amino]-3-pyridyl]-6-(5-methyl-1H-indazol-4-yl)pyrimidine-4-carboxamide (45 mg, 51% yield). MS: [M+H]⁺: 493.2; ¹H NMR (DMSO-d₆) δ 13.25 (s, 1H), 11.08 (s, 1H), 8.76 (dd, J=7.9, 1.9 Hz, 1H), 8.46 (s, 1H), 8.40 (dd, J=4.7, 1.9 Hz, 1H), 8.22 (s, 1H), 7.84 (td, J=8.9, 6.4 Hz, 1H), 7.75 (s, 1H), 7.65 (dd, J=8.6, 1.0 Hz, 1H), 7.40 (d, J=8.6 Hz, 1H), 7.15 (dd, J=7.9, 4.7 Hz, 1H), 6.85 (ddd, J=8.6, 3.5, 2.3 Hz, 1H), 2.29 (s, 3H).

### Chiral Separation of Selected Compounds

Racemic mixtures of atropisomers were separated using chiral SFC methods on, for example, a Mettler Toledo Minigram SFC (MTM), a Waters Prep 15 SFC-MS (WP15), a Waters Prep 100 SFC-MS (WP100) or a Pic Solution Hybrid 10-150 (PSH). An appropriate column was selected to achieve a satisfactory resolution of the peaks. The appropriate fractions for each peak were combined, concentrated and usually taken in a mixture of water and a suitable water miscible organic solvent such as EtOH, IPA, CH₃CN or a mixture thereof and freeze-dried. The separated products were reanalyzed by chiral SFC to assess chiral purity.

Exemplary columns used for chiral separations include, for example, Phenomenex Lux Cellulose-2, 10×250 mm, 5 μm; Phenomenex Lux Cellulose-2, 30×250 mm, 5 μm; Chiral Technologies IA, 10×250 mm, 5 μm; Chiral Technologies IC, 10×250 mm, 5 μm; Chiral Technologies ID, 10×250 mm, 5 μm; Chiral Technologies IG, 10×250 mm, 5 μm; Chiral Technologies AS, 10×250 mm, 5 μm; Phenomenex Lux Cellulose-4, 10×250 mm, 5 μm; Phenomenex Lux Cellulose-1, 21.2×250 mm, 5 μm.

Structural assignments of the separated atropisomers were confirmed by biological activity where the biologically active enantiomer was assigned to have the (S) configuration, which was confirmed by X-ray crystallography of key compounds.

### Example 2. Enzymatic Assay

Detection of Myt1 kinase activity utilized a recombinant human Myt1 kinase assay measuring the hydrolysis of ATP using a commercially available ADP-Glo Assay (ADP-Glo™ Kinase Assay from Promega, 10 000 assays, #V9102). Briefly, 5 μL recombinant human Myt1 (full length PKMYT1 recombinant human protein expressed in insect cells from Thermo Fisher #A33387; ˜80% purity) was prepared in reaction buffer (70 mM HEPES, 3 mM MgCl₂, 3 mM MnCl₂, 50 μg/ml PEG 20000, 3 μM Na-orthovanadate, 1.2 mM DTT) and added to 384 well white polystyrene, flat bottom well, non-treated, microplate (Corning #3572). After this, 5 μL of compounds (diluted in reaction buffer to 0.5% DMSO) was added to the microplate and the plate was spun briefly and incubated at 22° C. for 15 minutes. Ultra-Pure Adenosine Triphosphate (ATP) solution (ADP-Glo kit from Promega) was diluted in reaction buffer and 5 μL was added to the microplate, spun down briefly and incubated for 60 minutes at 30° C. The final Myt1 enzyme concentration was 18 nM and the final ATP concentration was 10 μM. After the 60-minute incubation, 15 μL of ADP-Glo reagent was added and the plate was spun briefly and sealed and incubated in the dark for 40 minutes at 22° C. Following this, 30 μL of kinase detection reagent was added per well and the plate was spun briefly, sealed and incubated for 45-60 minutes at 22° C. in the dark. Luminescence was read using the Envision (250 ms integration). The IC₅₀ and the % max inhibition were calculated for each inhibitor compound tested.

To determine compound IC50 (PKMYT1 cell-based activity assay, CDK1 pThr14 AlphaLISA), FUOV1 cells were plated into a 96-well TC-treated culture plate at 50000 cells/well in a final volume of 100 μL of media. The plates were then allowed to equilibrate in a biological safety cabinet for 30 minutes before being placed in a humidified incubator at 37 C and 5% CO₂ overnight. The next day, 2 μL of PKMYT1 inhibitors or DMSO were diluted in 400 μL of warmed culture media in a 96-well block using a Biomek FX liquid handler. Compounds were mixed in media and then 25 μL was dispensed into each well of the 96-well cell plate. Plates were centrifuged at 300 g for 10 seconds and then placed in the incubator for 2 hours. After the 2-hour incubation with compound, media was removed via aspiration using a multichannel pipette. 30 μL of 1× AlphaLISA lysis buffer (Perkin Elmer) supplemented with protease and phosphatase inhibitors as well as 1 mM PMSF, was added to each well. Plates were rotated at 500 g for 20 minutes to facilitate lysis. Plates were then sealed with aluminum foil and frozen at −80° C. for at least 1 hour. Lysates were thawed at 37° C. for 10 minutes, then 10 μL of each lysate was transferred in duplicate to a white 384-well assay plate. Antibody mixture was prepared in 1× AlphaLISA assay buffer (Perkin Elmer) containing antibodies (5 nM final concentration for rabbit pThr14-CDK1 from Abcam #ab58509 and mouse total CDK1 from ThermoFisher Scientific #33-1800). 5 μL of antibody mixture was added to each well of the assay plate. The assay plate was sealed and stored at 4° C. overnight. The next day, AlphaLISA bead mixture (Perkin Elmer) was prepared in 1× AlphaLISA assay buffer. Anti-rabbit IgG Acceptor (Perkin Elmer #AL104C) and anti-mouse IgG Donor beads (Perkin Elmer #AS104D) were prepared to a concentration of 80 μg/ml in assay buffer. 5 μL of bead mixture was added to each well of the assay plate (20 μg/ml final concentration for each bead). The plate was protected from light and incubated for 2 hours at room temperature. After a 2-hour incubation with beads, the plate was read using the Perkin Elmer EnVision Multimode plate reader with excitation at 680 nm and emission at 615 nm.

Exemplary prepared compounds and their activities are shown in Table 2 below. In Table 2, the Method column indicates a preparatory method described above used in the preparation of the compounds.

TABLE 2

| | | | FUOV-1 CDK1 | MS (+ESI) |
|---|---|---|---|---|
| | | Myt1 IC₅₀ | pThr14 IC₅₀ | [M + 1] |
| Compound | Method | (nM) | (nM) | (m/z) |
| 1 | A | >50000 | | 294.1 |
| 2 | B | 11 | 517 | 321.2 |
| 3 | B | 21 | 560 | 328.2 |
| 4 | B | 10 | 943 | 337.3 |
| 5 | B | 25 | | 340.2 |
| 6 | B | 24 | 1010 | 449.5 |
| 7 | B | 31 | 737 | 389.5 |
| 8 | B | 25 | | 364.5 |
| 9 | B | 44 | | 342.2 |
| 10 | BC | 202 | | 294.3 |
| 11 | B | 54 | | 350.4 |
| 12 | B | 246 | | 351.2 |
| 13 | C | <3.5 | 64 | 341.2 |
| 14 | C | 4 | 132 | 350.2 |
| 15 | C | 20 | 2520 | 402.2 |
| 16 | C | 5 | 575 | 370.2 |
| 17 | C | 36 | 8670 | 292.2 |
| 18 | B | 28 | 3530 | 355.2 |
| 19 | C | 4 | 14 | 335.2 |
| 20 | B | 105 | | 335.3 |
| 21 | B | 202 | | 322.3 |
| 22 | B | 18 | | 357.2 |
| 23 | B | 51 | | 346.3 |
| 24 | B | 18 | | 322.2 |
| 25 | B | 15 | | 352.2 |
| 26 | C | 3 | 76 | 336.2 |
| 27 | B | 11 | 319 | 406.4 |
| 28 | B | 45 | | 373.2 |
| 29 | B | 25 | 1220 | 355.2 |
| 30 | B | 74 | | 335.2 |
| 31 | B | 341 | | 335.2 |
| 32 | B | 175 | | 407.0 |
| 33 | B | 52 | | 337.0 |
| 34 | B | 81 | | 325.2 |
| 35 | B | 167 | | 325.2 |
| 36 | B | 92 | | 352.2 |
| 37 | B | 42 | | 406.3 |
| 38 | C | 3 | 60 | 341.2 |
| 39 | C | 7 | 266 | 371.2 |
| 40 | C | 4 | 208 | 349.2 |
| 41 | BC | 827 | | 363.3 |
| 42 | C | 7 | 169 | 419.2 |
| 43 | C | 6 | 336 | 324.2 |
| 44 | BC | 3180 | | 328.6 |
| 45 | C | 2 | 60 | 350.2 |
| 46 | BD | 62 | | 387.4 |
| 47 | BB | | 13 | 499.1 |
| 48 | BD | 55 | 7740 | 385.4 |
| 49 | C | 6 | 137 | 391.3 |
| 50 | C | 6 | 368 | 324.3 |
| 51 | C | 8 | 357 | 377.4 |
| 52 | C | 8 | 45 | 336.3 |
| 53 | D | 1830 | | 302.2 |
| 54 | BD | 18 | 2410 | 298.3 |
| 55 | BD | 15 | 146 | 391.4 |
| 56 | BD | 7 | 373 | 375.4 |
| 57 | BD | 7 | 46 | 435.4 |
| 58 | BD | 5 | 387 | 391.3 |
| 59 | BD | 4 | 59 | 365.4 |
| 60 | BD | 11 | 1820 | 403.4 |
| 61 | BD | 2 | 68 | 365.4 |
| 62 | BD | 8 | 117 | 365.4 |
| 63 | BD | 5 | 40 | 336.3 |
| 64 | K | 6 | 260 | 349.4 |
| 65 | L | 22 | 1070 | 345.4 |
| 66 | BD | 2 | 168 | 365.4 |
| 67 | BD | 2 | 272 | 403.3 |
| 68 | L | 50 | >10000 | 331.5 |
| 69 | E | 131 | | 332.3 |
| 70 | E | 29 | 1470 | 331.3 |
| 71 | BD | 9 | 298 | 336.3 |
| 72 | C | 10 | 77 | 351.3 |
| 73 | F | >50 | | 313.1 |
| 74 | G | 2880 | | 279.1 |
| 75 | H | >2000 | | 364.1 |
| 76 | I | >2000 | | 330.1 |
| 77 | J | 27 | 179 | 336.3 |
| 78 | K | 9 | 326 | 335.3 |
| 79 | C | 8 | 90 | 336.3 |
| 80 | K | 8 | 259 | 334.5 |
| 81 | K | 5 | 112 | 340.2 |
| 82 | C | 7 | 107 | 341.3 |
| 83 | C | 6 | 188 | 349.4 |
| 84 | K | 10 | 357 | 335.4 |
| 85 | E | 22 | 1450 | 351.3 |
| 86 | E | 58 | 945 | 346.3 |
| 87 | E | 11 | 567 | 345.3 |
| 88 | J | 7 | 401 | 336.3 |
| 89 | C | 3 | 16 | 336.3 |
| 90 | C | 3 | 58 | 341.3 |
| 91 | C | 3 | 113 | 349.4 |
| 92 | K | 5 | 131 | 340.4 |
| 93 | K | 25 | 937 | 335.2 |
| 94 | C | 2 | 31 | 435.5 |
| 95 | E | 16 | 1700 | 346.3 |
| 96 | E | 387 | | 345.3 |
| 97 | E | 54 | | 345.4 |
| 98 | E | 1940 | | 351.3 |
| 99 | L | 59 | | 346.5 |
| 100 | L | 27 | 1230 | 346.3 |
| 101 | L | 26 | 570 | 359.4 |
| 102 | L | 19 | 462 | 430.7 |
| 103 | L | 94 | | 430.4 |
| 104 | L | 317 | | 413.5 |
| 105 | E | 12 | 551 | 346.3 |
| 106 | E | 28 | | 366.3 |
| 107 | E | 11 | 495 | 345.4 |
| 108 | L | 79 | 7400 | 352.3 |
| 109 | L | 23 | 717 | 352.3 |
| 110 | L | 23 | 883 | 365.1 |
| 111 | L | 44 | | 379.3 |
| 112 | L | 100 | | 413.3 |
| 113 | L | 1110 | | 437.4 |
| 114 | L | 46 | 3760 | 399.4 |
| 115 | M | 15 | 2230 | 365.4 |
| 116 | M | >5000 | | 345.2 |
| 117 | L | 90 | | 346.3 |
| 118 | L | 309 | | 470.7 |
| 119 | L | 25 | | 399.6 |
| 120 | N | 329 | | 346.4 |
| 121 | M | 49 | >10000 | 349.4 |
| 122 | M | 137 | 820 | 349.4 |
| 123 | M | 99 | >10000 | 349.3 |
| 124 | L | 25 | | 419.3 |
| 125 | L | 51 | | 410.6 |
| 126 | L | 27 | | 412.6 |
| 127 | L | 120 | | 401.5 |
| 128 | L | 57 | | 387.5 |
| 129 | L | 23 | | 385.5 |
| 130 | L | 66 | | 384.4 |
| 131 | L | 22 | 1370 | 370.3 |
| 132 | L | 37 | | 442.5 |
| 133 | L | 39 | | 369.4 |
| 134 | L | 142 | | 442.5 |
| 135 | L | 22 | 1150 | 375.4 |
| 136 | L | 25 | | 359.4 |
| 137 | O | 46 | | 344.3 |
| 138 | L | 43 | | 433.7 |
| 139 | L | 57 | | 388.3 |
| 140 | L | 48 | | 374.3 |
| 141 | L | 23 | | 375.4 |
| 142 | L | 54 | | 414.4 |
| 143 | L | 23 | 1470 | 389.3 |
| 144 | L | 130 | | 427.4 |
| 145 | L | 57 | | 388.6 |
| 146 | L | 23 | >10000 | 389.3 |
| 147 | L | 217 | | 444.4 |
| 148 | L | 69 | | 365.2 |
| 149 | K | 735 | | 335.1 |
| 150 | L | 1110 | | 389.5 |
| 151 | L | 27 | | 384.3 |
| 152 | L | 44 | | 374.4 |
| 153 | L | 27 | | 414.3 |
| 154 | L | 30 | | 444.5 |
| 155 | L | 15 | | 431.6 |
| 156 | L | 17 | | 450.6 |
| 157 | L | 904 | | 498.4 |
| 158 | L | 21 | | 376.3 |
| 159 | P | 6 | 268 | 444.6 |
| 160 | P | 7 | 388 | 404.6 |
| 161 | P | 7 | 122 | 418.0 |
| 162 | L | 198 | | 395.5 |
| 163 | Q | 11 | 436 | 401.3 |
| 164 | P | 14 | 473 | 453.7 |
| 165 | P | 586 | >10000 | 444.7 |
| 166 | P | 3 | 109 | 420.7 |
| 167 | P | 8 | 414 | 392.6 |
| 168 | P | 29 | 7590 | 424.4 |
| 169 | P | 8 | 120 | 446.7 |
| 170 | P | 23 | 1100 | 430.7 |
| 171 | E | 96 | | 348.6 |
| 172 | E | 53 | | 348.6 |
| 173 | P | 6 | 278 | 438.4 |
| 174 | Q | 10 | 939 | 415.7 |
| 175 | E | 364 | | 442.7 |
| 176 | P | 770 | | 418.2 |
| 177 | BC | 13 | 346 | 454.4 |
| 178 | E | 68 | | 361.3 |
| 179 | O | 7 | 423 | 452.2 |
| 180 | R | 10 | 99 | 458.3 |
| 181 | R | 103 | | 481.2 |
| 182 | R | 15 | 1600 | 506.3 |
| 183 | R | 13 | 281 | 473.4 |
| 184 | R | 6 | 51 | 453.3 |
| 185 | R | 7 | 369 | 474.4 |
| 186 | E | 47 | 1690 | 428.2 |
| 187 | V | 78 | | 446.4 |
| 188 | D | 8 | 266 | 378.2 |
| 189 | E | 21 | 1220 | 360.1 |
| 190 | E | 20 | 860 | 346.1 |
| 191 | E | 42 | 390 | 346.1 |
| 192 | P | 25 | | 477.5 |
| 193 | P | 191 | | 517.5 |
| 194 | P | 10 | 476 | 417.4 |
| 195 | P | 182 | | 482.8 |
| 196 | P | 754 | | 492.4 |
| 197 | P | 12 | 267 | 434.4 |
| 198 | Q | 9 | 970 | 431.5 |
| 199 | P | 6 | 137 | 439.7 |
| 200 | Q | 11 | 154 | 438.5 |
| 201 | E | 91 | | 417.7 |
| 202 | R | 107 | | 438.7 |
| 203 | P | 11 | 332 | 434.7 |
| 204 | P | 6 | 205 | 434.4 |
| 205 | P | 6 | 181 | 446.4 |
| 206 | P | 8 | 554 | 496.8 |
| 207 | P | 9 | 144 | 464.4 |
| 208 | P | 6 | 106 | 474.4 |
| 209 | P | 73 | | 519.8 |
| 210 | P | 10 | 335 | 419.4 |
| 211 | P | 7 | 184 | 446.2 |
| 212 | P | 12 | 192 | 432.4 |
| 213 | P | 7 | 404 | 442.8 |
| 214 | Q | 5 | 444 | 419.4 |
| 215 | Q | 7 | 399 | 472.9 |
| 216 | R | 1510 | | 324.2 |
| 217 | E | 13 | | 430.2 |
| 218 | E | 230 | | 409.1 |
| 219 | E | 56 | | 346.1 |
| 220 | P | 4 | 150 | 432.5 |
| 221 | P | 7 | 270 | 448.4 |
| 222 | T | >2000 | | 359.1 |
| 223 | E | 61 | | 423.1 |
| 224 | E | 40 | 252 | 423.1 |
| 225 | E | 161 | | 409.2 |
| 226 | R | 4 | 367 | 431.3 |
| 227 | R | 2 | 198 | 431.3 |
| 228 | R | 6 | 387 | 452.3 |
| 229 | P | 4 | 134 | 453.5 |
| 230 | P | 4 | 208 | 453.4 |
| 231 | P | 231 | | 446.4 |
| 232 | P | 913 | | 421.0 |
| 233 | P | 859 | | 459.8 |
| 234 | R | 1880 | | 453.1 |
| 235 | P | 7 | 39 | 454.1 |
| 236 | E | 46 | | 422.8 |
| 237 | E | 48 | | 360.7 |
| 238 | P | 5 | 92 | 496.9 |
| 239 | P | 4 | 903 | 466.8 |
| 240 | P | 8 | 996 | 456.2 |
| 241 | R | 10 | 1970 | 363.2 |
| 242 | R | 7 | 243 | 453.3 |
| 243 | R | 73 | | 361.2 |
| 244 | R | 11 | 193 | 453.3 |
| 245 | R | 10 | 539 | 419.2 |
| 246 | U | 720 | | 345.1 |
| 247 | R | 17 | | 465.3 |
| 248 | R | 71 | | 455.3 |
| 249 | R | 5 | 85 | 455.3 |
| 250 | R | 8 | 752 | 453.3 |
| 251 | P | 3 | 46 | 454.3 |
| 252 | R | 26 | | 466.2 |
| 253 | R | 55 | | 467.3 |
| 254 | R | 5 | 204 | 467.3 |
| 255 | R | 72 | | 483.3 |
| 256 | R | 21 | 132 | 495.3 |
| 257 | E | 12 | 1170 | 379.1 |
| 258 | R | >2000 | | 541.3 |
| 259 | R | 584 | | 538.3 |
| 260 | R | 4 | 167 | 453.3 |
| 261 | R | 6 | 544 | 458.1 |
| 262 | R | 7 | 507 | 453.1 |
| 263 | P | 19 | | 467.3 |
| 264 | P | 9 | | 470.8 |
| 265 | P | 4 | 387 | 456.7 |
| 266 | P | 3 | 93 | 439.8 |
| 267 | R | 4 | 191 | 442.3 |
| 268 | R | 9 | 261 | 431.3 |
| 269 | E | 18 | | 359.2 |
| 270 | R | 1160 | | 419.2 |
| 271 | R | 1090 | | 458.2 |
| 272 | P | 12 | | 467.8 |
| 273 | R | 716 | 4560 | 442.3 |
| 274 | V | 3 | 136 | 480.4 |
| 275 | V | 141 | | 480.4 |
| 276 | R | 2 | 11 | 480.3 |
| 277 | R | 162 | | 480.4 |
| 278 | R | 5 | 158 | 458.1 |
| 279 | R | 8 | | 458.1 |
| 280 | E | 11 | | 359.2 |
| 281 | L | 8 | | 384.2 |
| 282 | L | 13 | | 411.3 |
| 283 | L | 30 | | 444.3 |
| 284 | L | 38 | | 416.3 |
| 285 | L | 20 | | 402.3 |
| 286 | L | 41 | | 419.3 |
| 287 | L | 62 | | 455.3 |
| 288 | L | 8 | 652 | 400.3 |
| 289 | L | 18 | | 458.3 |
| 290 | L | 6 | 167 | 427.3 |
| 291 | L | 25 | | 402.3 |
| 292 | L | 18 | | 389.3 |
| 293 | L | 16 | | 447.3 |
| 294 | L | 17 | | 419.3 |
| 295 | W | 27 | | 452.3 |
| 296 | W | 213 | | 520.3 |
| 297 | L | 9 | 422 | 389.2 |
| 298 | L | 15 | | 402.3 |
| 299 | P | 13 | 232 | 406.3 |
| 300 | P | 100 | | 512.4 |
| 301 | P | 4 | 39 | 456.8 |
| 302 | P | 4 | 23 | 448.3 |
| 303 | P | 7 | 155 | 434.3 |
| 304 | P | 27 | | 373.2 |
| 305 | P | 81 | | 375.2 |
| 306 | R | 7 | 449 | 445.1 |
| 307 | R | 25 | | 487.2 |
| 308 | P | 1740 | | 496.1 |
| 309 | P | 5 | 22 | 496.1 |
| 310 | P | 14 | | 468.4 |
| 311 | P | >2000 | | 468.8 |
| 312 | P | 9 | 596 | 470.4 |
| 313 | P | 6 | 137 | 473.7 |
| 314 | BC | | 1620 | 493.2 |
| 315 | P | 9 | 349 | 456.4 |
| 316 | P | 20 | | 457.4 |
| 317 | P | 13 | | 459.4 |
| 318 | R | 21 | | 457.3 |
| 319 | L | 48 | | 360.2 |
| 320 | L | 6 | 19 | 402.3 |
| 321 | T | >2000 | | 381.1 |
| 322 | V | 10 | 164 | 430.3 |
| 323 | L | 6 | 108 | 385.3 |
| 324 | R | 10 | 303 | 441.8 |
| 325 | U | >2000 | | 399.1 |
| 326 | R | 5 | 134 | 453.1 |
| 327 | P | 6 | 351 | 448.3 |
| 328 | P | 18 | | 524.9 |
| 329 | P | 14 | | 456.4 |
| 330 | P | 88 | | 468.4 |
| 331 | V | 54 | | 478.2 |
| 332 | L, Y | 6 | 56 | 432.3 |
| 333 | P | 3 | 21 | 448.2 |
| 334 | P | 400 | 1180 | 448.2 |
| 335 | X | 195 | | 359.4 |
| 336 | P | 65 | | 506.8 |
| 337 | P | 19 | | 459.4 |
| 338 | R | 165 | 284 | 480.2 |
| 339 | R | 1160 | | 481.4 |
| 340 | BC | >2000 | | 451.7 |
| 341 | Q | 16 | | 431.0 |
| 342 | P | 3 | 6 | 481.1 |
| 343 | Q | 20 | | 419.3 |
| 344 | R | 7 | 983 | 459.3 |
| 345 | R | 1020 | | 446.2 |
| 346 | R | 13 | | 459.6 |
| 347 | Q | 18 | | 431.0 |
| 348 | Q | 15 | | 431.4 |
| 349 | Q | 138 | | 521.5 |
| 350 | R | 35 | | 445.3 |
| 351 | R | 149 | | 494.1 |
| 352 | R | 74 | | 432.3 |
| 353 | R | 7 | 20 | 430.3 |
| 354 | L | 293 | | 495.1 |
| 355 | R | 7 | | 472.3 |
| 356 | R | 9 | | 445.2 |
| 357 | P | 5 | | 468.0 |
| 358 | P | 69 | | 468.0 |
| 359 | Q | 4 | | 445.1 |
| 360 | E | 330 | | 446.4 |
| 361 | R | 35 | | 431.2 |
| 362 | Q | 2 | | 481.1 |
| 363 | R | 4 | | 459.2 |
| 364 | R | 8 | | 459.2 |
| 365 | L | 28 | 774 | 374.2 |
| 366 | L | 28 | | 384.2 |
| 367 | Q | 9 | | 445.1 |
| 368 | Q | 15 | | 460.6 |
| 369 | Q | 4 | | 459.5 |
| 370 | Q | 869 | | 495.2 |
| 371 | Q | 466 | 2420 | 481.0 |
| 372 | Q | >2000 | | 492.9 |
| 373 | Q | 20 | | 445.2 |
| 374 | R | 14 | | 444.3 |
| 375 | R | 14 | | 449.0 |
| 376 | Q | 15 | | 493.1 |
| 377 | L | 3 | 21 | 444.3 |
| 378 | L | 18 | | 385.2 |
| 379 | L | 14 | | 401.2 |
| 380 | L | 6 | 772 | 402.2 |
| 381 | L, AA | 11 | 13 | 456.1 |
| 382 | R | 3 | | 430.2 |
| 383 | Q | 2 | | 495.2 |
| 384 | Q | 4 | | 493.1 |
| 385 | L | 5 | 289 | 400.2 |
| 386 | L | 29 | | 386.2 |
| 387 | R | 640 | | 444.3 |
| 388 | R | 643 | | 445.2 |
| 389 | L | 8 | 433 | 416.1 |
| 390 | L | 5 | | 403.1 |
| 391 | P | 5 | 56 | 460.2 |
| 392 | Q | 461 | | 507.2 |
| 393 | L | 6 | 288 | 418.1 |
| 394 | Q | 98 | | 447.0 |
| 395 | L | 17 | 780 | 436.2 |
| 396 | L | 5 | 152 | 402.1 |
| 397 | L, Y | 4 | 13 | 428.2 |
| 398 | N | 7 | 28 | 443.2 |
| 399 | L | 17 | 143 | 426.2 |
| 400 | L, Z | 2 | 36 | 424.2 |
| 401 | L, Y | 5 | 4 | 442.2 |
| 402 | L, Y | 6 | 41 | 498.1 |
| 403 | L | 2 | 14 | 464.1 |
| 404 | Q | 24 | | 472.8 |
| 405 | N, Y | 4 | 93 | 496.3 |
| 406 | N, AA | 5 | 53 | 455.1 |
| 407 | L, Y | 4 | | 430.2 |
| 408 | L, Y | 7 | 9 | 458.2 |
| 409 | L | 5 | 28 | 442.0 |
| 410 | L, Y | 4 | 9 | 462.1 |
| 411 | L, Y | 7 | 60 | 516.0 |
| 412 | N, Z | 4 | 223 | 423.2 |
| 413 | N | 5 | 184 | 441.1 |
| 414 | L | 122 | 2460 | 395.1 |
| 415 | L, Y | 8 | 210 | 478.1 |
| 416 | L, Y | 7 | 22 | 456.2 |
| 417 | L, Y | 18 | 899 | 470.2 |
| 418 | AB | 27 | 1330 | 357.2 |
| 419 | L, AA | 4 | 49 | 402.2 |
| 420 | L, Y | 7 | 65 | 460.1 |
| 421 | L, Z | 4 | 40 | 442.1 |
| 422 | L, Y | 19 | 373 | 471.2 |
| 423 | L, Y | 4 | 23 | 453.1 |
| 424 | L, Y | 4 | 81 | 472.1 |
| 425 | L, Y | 139 | >10000 | 492.1 |
| 426 | AC | 4 | 38 | 464.3 |
| 427 | AC | 4 | 23 | 438.3 |
| 428 | N | 3 | 26 | 431.2 |
| 429 | L, AA | 4 | 36 | 456.2 |
| 430 | L, AA | 8 | 8 | 456.1 |
| 431 | L, AA | 8 | 5 | 471.2 |
| 432 | L, AA | 5 | 105 | 458.1 |
| 433 | L, AA | 5 | 13 | 456.2 |
| 434 | L, AA | 3 | 8 | 448.1 |
| 435 | L, Z | 4 | 136 | 442.1 |
| 436 | L, Z | 7 | 206 | 460.1 |
| 437 | L, AA | 8 | 59 | 456.2 |
| 438 | L, AA | 4 | 17 | 476.1 |
| 439 | L, AA | 6 | 42 | 476.1 |
| 440 | L, AA | 13 | 523 | 456.2 |
| 441 | L, AA | 4 | 8 | 462.1 |
| 442 | L, Z | 4 | 33 | 388.3 |
| 443 | L, AA | 3 | 26 | 478.2 |
| 444 | L | 43 | | 394.2 |
| 445 | L, AA | 38 | 1030 | 510.3 |
| 446 | L, AA | 8 | 8 | 478.3 |
| 447 | L, AA | 3 | 5 | 460.3 |
| 448 | L, AA | 7 | 112 | 472.1 |
| 449 | AD | 7 | 23 | 438.3 |
| 450 | AD | 8 | 121 | 455.3 |
| 451 | L | 7 | 104 | 416.3 |
| 452 | L, AE | 16 | 124 | 428.3 |
| 453 | L, AA | 12 | 317 | 478.2 |
| 454 | AF | 26 | 447 | 510.2 |
| 455 | AF | 6 | 21 | 472.3 |
| 456 | AF | 6 | 158 | 460.3 |
| 457 | L, Y | 516 | >10000 | 456.2 |
| 458 | AG | 2 | 142 | 358.1 |
| 459 | AF | 9 | 44 | 498.2 |
| 460 | L, Y | 10 | 825 | 482.1 |
| 461 | L, Y | 3 | 6 | 446.2 |
| 462 | O | 4 | 137 | 492.2 |
| 463 | L, Y | 8 | 278 | 510.1 |
| 464 | L, Z | 3 | 370 | 418.3 |
| 465 | L | 5 | 233 | 461.3 |
| 466 | L, AH | 6 | 77 | 480.3 |
| 467 | L, Z | 6 | 106 | 460.2 |
| 468 | L, Z | 6 | 228 | 480.1 |
| 469 | L | 12 | 540 | 361.2 |
| 470 | AF | 18 | 1230 | 546.2 |
| 471 | AF | 16 | 261 | 532.2 |
| 472 | AD | 6 | 26 | 456.2 |
| 473 | L | 8 | 48 | 432.2 |
| 474 | L | 16 | 398 | 460.2 |
| 475 | L, AH | 14 | 138 | 486.2 |
| 476 | L | 3 | 25 | 400.3 |
| 477 | L, Y | 3 | 26 | 489.1 |
| 478 | L, Y | 6 | 80 | 468.2 |
| 479 | L, AH | >2000 | | 551.2 |
| 480 | AC | 5 | 158 | 500.2 |
| 481 | AC | 2 | 83 | 482.2 |
| 482 | AC | 5 | 114 | 496.2 |
| 483 | L | 2 | 2 | 445.3 |
| 484 | L, Y | 7 | 23 | 521.3 |
| 485 | L, AH | 7 | 53 | 493.3 |
| 486 | L, AH | | 114 | 451.2 |
| 487 | L, Y | | 68 | 480.2 |
| 488 | L, AH | | 861 | 492.2 |
| 489 | AD | | 22 | 456.2 |
| 490 | AD | | 51 | 456.2 |
| 491 | AC | | 45 | 456.3 |
| 492 | AD | | 163 | 474.3 |
| 493 | AC | | 681 | 492.3 |
| 494 | AD | | 144 | 456.2 |
| 495 | L, AH | | 198 | 461.2 |
| 496 | L, AH | | 465 | 487.2 |
| 497 | L, AH | | 52 | 452.2 |
| 498 | R | 93 | | 432.0 |
| 499 | AI | 4 | 209 | 459.3 |
| 500 | AJ | | 437 | 477.3 |
| 501 | L, AH | | 146 | 469.2 |
| 502 | L, AH | | 871 | 535.3 |
| 503 | L, AH | | 23 | 480.2 |
| 504 | L, Z | | 31 | 443.2 |
| 505 | AF | | 36 | 504.2 |
| 506 | AD | | 236 | 486.2 |
| 507 | N | | 166 | 444.3 |
| 508 | L, AH | | 622 | 498.2 |
| 509 | L, Y | | 285 | 550.1 |
| 510 | AD | | 871 | 495.2 |
| 511 | AD | | 25 | 456.2 |
| 512 | AC | | 52 | 456.3 |
| 513 | AD | | 16 | 456.2 |
| 514 | AC | | 1340 | 532.3 |
| 515 | AD | | 11 | 456.2 |
| 516 | AD | | 30 | 455.2 |
| 517 | L, AH | | 139 | 475.2 |
| 518 | AL | | 18 | 443.2 |
| 519 | L, AM | | 59 | 455.2 |
| 520 | AN | | 27 | 443.2 |
| 521 | L, AM | | 582 | 458.2 |
| 522 | L, AM | | 101 | 458.2 |
| 523 | AD | | 269 | 470.2 |
| 524 | AD | | 69 | 470.2 |
| 525 | AD | 5 | 19 | 470.2 |
| 526 | AD | | 4 | 474.3 |
| 527 | AD | | 11 | 473.3 |
| 528 | AF | | 118 | 490.4 |
| 529 | L, AH | | 251 | 528.1 |
| 530 | AC | | 59 | 484.2 |
| 531 | AC | | 918 | 510.2 |
| 532 | AD | | 21 | 481.3 |
| 533 | AD | | 3 | 455.2 |
| 534 | AD | | 87 | 470.2 |
| 535 | AD | | 151 | 481.3 |
| 536 | AP | | 8 | 443.2 |
| 537 | AL | | 256 | 433.3 |
| 538 | AP | | 33 | 442.9 |
| 539 | AD | | 20 | 481.2 |
| 540 | AD | | 9 | 470.2 |
| 541 | AO | | 47 | 456.3 |
| 542 | AL | | 77 | 414.3 |
| 543 | AL | | 931 | 467.2 |
| 544 | AL | | 6 | 459.3 |
| 545 | AL | | 43 | 443.2 |
| 546 | AL | | 12 | 445.3 |
| 547 | AL | | 50 | 431.5 |
| 548 | AL | | 227 | 445.5 |
| 549 | AL | | 1320 | 472.3 |
| 550 | AL | | 7 | 431.3 |
| 551 | AL | | 151 | 445.3 |
| 552 | AL | | 245 | 458.3 |
| 553 | AO | | 183 | 456.5 |
| 554 | AD | | 138 | 494.1 |
| 555 | AP | | 219 | 444.1 |
| 556 | AQ | | 132 | 432.1 |
| 557 | AQ | | 416 | 376.1 |
| 558 | AR | | 95 | 431.1 |
| 559 | AQ | | 1290 | 455.2 |
| 560 | AQ | | 7040 | 456.1 |
| 561 | AL | | 426 | 458.3 |
| 562 | AK | | 24 | 438.4 |
| 563 | AK | | 38 | 453.4 |
| 564 | AD | | 15 | 469.5 |
| 565 | AK | | 71 | 453.5 |
| 566 | AD | | 76 | 486.5 |
| 567 | AD | | 389 | 484.2 |
| 568 | AD | | 15 | 488.2 |
| 569 | AQ | | 712 | 465.1 |
| 570 | AD | | 111 | 486.3 |
| 571 | AC | | 283 | 496.2 |
| 572 | AD | | 764 | 524.2 |
| 573 | AP | | 182 | 444.1 |
| 574 | AQ | | 1330 | 444.1 |
| 575 | AR | | 24 | 457.2 |
| 576 | AD | | 4580 | 512.3 |
| 577 | AD | | 702 | 541.2 |
| 578 | AD | | 150 | 484.3 |
| 579 | AD | | 13 | 462.1 |
| 580 | AS | | 44 | 479.2 |
| 581 | AD | | 40 | 461.2 |
| 582 | AL | | 15 | 445.3 |
| 583 | AD | | 122 | 502.1 |
| 584 | AD | | 18 | 483.2 |
| 585 | AD | | 127 | 523.2 |
| 586 | AD | | 75 | 494.2 |
| 587 | AD | | 172 | 475.2 |
| 588 | AD | | 45 | 475.2 |
| 589 | AS | | 111 | 472.2 |
| 590 | AS | | 8 | 480.3 |
| 591 | AL | | 22 | 445.3 |
| 592 | AL | | 603 | 473.3 |
| 593 | AL | | 297 | 459.3 |
| 594 | AL | | 36 | 445.2 |
| 595 | AL | | 1540 | 438.3 |
| 596 | AL | | 72 | 473.3 |
| 597 | AL | | 49 | 461.3 |
| 598 | L | 27 | | 361.3 |
| 599 | AL | 363 | 630 | 452.3 |
| 600 | AL | 10 | 885 | 455.3 |
| 601 | AL | 3 | 467 | 445.3 |
| 602 | AL | 4 | 29 | 445.3 |
| 603 | AL | 4 | 25 | 459.3 |
| 604 | AL | 16 | 794 | 459.3 |
| 605 | AL | 4 | 283 | 459.3 |
| 606 | AL | 2 | 67 | 459.3 |
| 607 | AL | 6 | 319 | 437.3 |
| 608 | AL | 29 | 1390 | 505.2 |
| 609 | AL | 4 | 414 | 480.3 |
| 610 | AL | 10 | 397 | 455.3 |
| 611 | AD | 21 | 22 | 489.5 |
| 612 | AD | 12 | 214 | 480.5 |
| 613 | AD | 4 | 6 | 480.5 |
| 614 | AD | 10 | 37 | 485.5 |
| 615 | AD | 16 | 515 | 496.5 |
| 616 | AD | 11 | 192 | 469.5 |
| 617 | AD | 4 | 7 | 470.5 |
| 618 | AD | 3 | 5 | 495.5 |
| 619 | AD | 6 | 277 | 497.6 |
| 620 | AD | 3 | 41 | 480.3 |
| 621 | AK | 3 | 3 | 456.5 |
| 622 | AK | 4 | 10 | 452.4 |
| 623 | AK | 4 | 179 | 452.5 |
| 624 | AD | 8 | 489 | 483.5 |
| 625 | AL | 5 | 42 | 425.3 |
| 626 | AK | 5 | 530 | 452.5 |
| 627 | AD | | 12 | 504.5 |
| 628 | AK | | 41 | 467.5 |
| 629 | AL | | 160 | 471.2 |
| 630 | AL | | 150 | 462.2 |
| 631 | AL | | 96 | 516.2 |
| 632 | AL | | 601 | 467.2 |
| 633 | AD | | 998 | 513.3 |
| 634 | AL | | 569 | 451.3 |
| 635 | AQ | | 402 | 458.2 |
| 636 | AD | | 97 | 468.2 |
| 637 | AD | | 241 | 472.1 |
| 638 | AL | | 707 | 471.2 |
| 639 | AD | | 121 | 495.2 |
| 640 | AK | | 2 | 470.5 |
| 641 | AK | | 392 | 474.4 |
| 642 | AL | | 472 | 461.2 |
| 643 | AL | | 7 | 463.2 |
| 644 | AL | | 10 | 463.2 |
| 645 | AD | | 12 | 463.3 |
| 646 | AD | | 91 | 452.2 |
| 647 | AL | | 28 | 463.2 |
| 648 | AL | | 51 | 463.2 |
| 649 | AL | | >10000 | 431.1 |
| 650 | AL | | 179 | 431.1 |
| 651 | AL | | 130 | 459.2 |
| 652 | AR | | 266 | 443.1 |
| 653 | AD | | 61 | 452.2 |
| 654 | AD | | 77 | 472.1 |
| 655 | AD | | 4 | 481.2 |
| 656 | AL | | 527 | 467.3 |
| 657 | AL | | 70 | 451.3 |
| 658 | AK | 5 | 8 | 467.3 |
| 659 | AK | | 185 | 456.5 |
| 660 | AK | | 2 | 474.4 |
| 661 | AD | | 383 | 445.2 |
| 662 | AL | | >10000 | 431.1 |
| 663 | AS | | 359 | 472.3 |
| 664 | AS | | 41 | 469.3 |
| 665 | AS | | 41 | 473.3 |
| 666 | AS | | 38 | 469.3 |
| 667 | AL | | 42 | 459.2 |
| 668 | AR | | 94 | 443.1 |
| 669 | AT | | >10000 | 444.2 |
| 670 | AL | | 198 | 445.2 |
| 671 | AS | | 164 | 469.2 |
| 672 | AK | | 33 | 470.2 |
| 673 | AS | | 1710 | 480.2 |
| 674 | AS | | 27 | 480.2 |
| 675 | AL | | 53 | 451.1 |
| 676 | AL | | >10000 | 431.1 |
| 677 | AD | | 451 | 492.1 |
| 678 | AL | | 31 | 469.2 |
| 679 | AL | | 13 | 421.2 |
| 680 | AL | | 14 | 419.1 |
| 681 | AL | | 11 | 429.2 |
| 682 | AL | | 83 | 433.2 |
| 683 | AL | | 65 | 451.2 |
| 684 | AD | | 122 | 477.1 |
| 685 | AL | | 12 | 449.1 |
| 686 | AL | | 10 | 463.2 |
| 687 | AK | | 3 | 466.3 |
| 688 | AK | | 180 | 467.5 |
| 689 | AK | | 6 | 481.5 |
| 690 | AL | | 16 | 448.8 |
| 691 | AL | | 228 | 463.1 |
| 692 | AL | | 195 | 448.8 |
| 693 | AD | | 641 | 491.1 |
| 694 | AS | | 423 | 498.3 |
| 695 | AS | | 41 | 470.3 |
| 696 | AS | | 69 | 486.3 |
| 697 | AK | | 2 | 470.3 |
| 698 | AS | | 1610 | 520.3 |
| 699 | AS | | 48 | 480.3 |
| 700 | BC | | 9 | 433.3 |
| 701 | AS | | 25 | 481.3 |
| 702 | AL | | 14 | 460.2 |
| 703 | AL | | 680 | 474.2 |
| 704 | AL | | 17 | 449.8 |
| 705 | AL | | 33 | 458.8 |
| 706 | AS | | 56 | 481.3 |
| 707 | AK | | 38 | 466.4 |
| 708 | AK | | >10000 | 478.3 |
| 709 | AK | | 18 | 482.3 |
| 710 | AL | | 19 | 446.8 |
| 711 | AL | | 14 | 448.8 |
| 712 | AL | | 66 | 434.8 |
| 713 | AL | | >10000 | 475.2 |
| 714 | AS | | 13 | 488.3 |
| 715 | BC | | 9 | 478.4 |
| 716 | AK | | 13 | 486.3 |
| 717 | AK | | 216 | 453.4 |
| 718 | AK | | 39 | 467.4 |
| 719 | AS | | 55 | 484.4 |
| 720 | AK | | 7 | 488.3 |
| 721 | AK | | 4 | 487.1 |
| 722 | AL | | 232 | 491.1 |
| 723 | AL | | 22 | 491.8 |
| 724 | AK | | 13 | 470.3 |
| 725 | AK | | 474 | 487.3 |
| 726 | AK | | 120 | 439.3 |
| 727 | BC | | 3 | 443.3 |
| 728 | AK | | 56 | 453.3 |
| 729 | AK | | 57 | 478.4 |
| 730 | AK | | 19 | 466.4 |
| 731 | AL | | 773 | 497.8 |
| 732 | AL | | 534 | 461.8 |
| 733 | AL | | 48 | 463.3 |
| 734 | AT | | 87 | 443.4 |
| 735 | AT | | 51 | 480.4 |
| 736 | AK | | 6 | 486.2 |
| 737 | AK | | 34 | 477.4 |
| 738 | AK | | 383 | 466.4 |
| 739 | AS | | 9 | 480.3 |
| 740 | AK | | 7 | 477.4 |
| 741 | AK | | 155 | 477.3 |
| 742 | AK | | 5 | 486.3 |
| 743 | AK | | 3 | 497.3 |
| 744 | AK | | 8 | 487.3 |
| 745 | AK | | 140 | 464.4 |
| 746 | AK | | 11 | 481.3 |
| 747 | AT | | 87 | 460.1 |
| 748 | AL | | 40 | 463.2 |
| 749 | AK | | 6 | 487.9 |
| 750 | AS | | 73 | 491.3 |
| 751 | AS | | 14 | 477.4 |
| 752 | AK | | 2 | 490.3 |
| 753 | AK | | 59 | 457.3 |
| 754 | AS | | 17 | 476.3 |
| 755 | AV | | 103 | 498.4 |
| 756 | AL | | 160 | 492.5 |
| 757 | AU | | 199 | 518.6 |
| 758 | AU | | 83 | 457.3 |
| 759 | AU | | 33 | 473.3 |
| 760 | AK | | 201 | 507.3 |
| 761 | AV | | 75 | 480.5 |
| 762 | AR | | 24 | 457.3 |
| 763 | AK | | 29 | 473.2 |
| 764 | AK | | 25 | 453.3 |
| 765 | AS | | 23 | 463.3 |
| 766 | AS | 3 | 41 | 477.3 |
| 767 | AK | | 11 | 506.2 |
| 768 | AK | | 122 | 438.3 |
| 769 | AT | | 230 | 456.3 |
| 770 | AK | | 17 | 507.3 |
| 771 | AK | | 96 | 541.3 |
| 772 | AT | | 113 | 479.3 |
| 773 | AK | | 6 | 487.9 |
| 774 | AT | | 563 | 452.3 |
| 775 | AT | | 2600 | 466.3 |
| 776 | AT | | 1130 | 470.3 |
| 777 | L | | >10000 | 375.2 |
| 778 | L | | 1260 | 373.3 |
| 779 | L | | 494 | 375.3 |
| 780 | AK | | 25 | 487.3 |
| 781 | AK | | 5 | 490.3 |
| 782 | L, AH | | 25 | 469.3 |
| 783 | AL | | 35 | 445.2 |
| 784 | AL | | 44 | 445.2 |
| 785 | AL | | 22 | 445.2 |
| 786 | AL | | 9 | 445.2 |
| 787 | AT | 4 | 278 | 389.3 |
| 788 | AT | 44 | 1270 | 452.3 |
| 789 | AT | | 43 | 463.3 |
| 790 | AT | | 271 | 499.2 |
| 791 | AL | | 61 | 463.3 |
| 792 | L | 10100 | >10000 | 480.3 |
| 793 | L | 266 | | 417.3 |
| 794 | L | 53 | 641 | 389.2 |
| 795 | L | 2520 | >10000 | 438.2 |
| 796 | L | 15 | 781 | 387.2 |
| 797 | AL | | 48 | 463.2 |
| 798 | AK | | 5 | 490.2 |
| 799 | AW | | >10000 | 479.3 |
| 800 | L | 1010 | | 437.0 |
| 801 | AW | | >10000 | 479.2 |
| 802 | AW | | 743 | 485.2 |
| 803 | AW | | 74 | 479.3 |
| 804 | BC | 8 | 62 | 439.4 |
| 805 | BC | 3 | 71 | 445.3 |
| 806 | BC | | >10000 | 475.3 |
| 807 | L | 4050 | >10000 | 416.3 |
| 808 | AT | 14 | >10000 | 411.3 |
| 809 | AW | | 272 | 476.3 |
| 810 | AW | | 96 | 470.3 |
| 811 | BA | | 30 | 482.1 |
| 812 | AW | | 26 | 463.2 |
| 813 | AW | | 123 | 465.3 |
| 814 | AW | | 71 | 465.3 |
| 815 | AW | | 511 | 465.3 |
| 816 | L | | >10000 | 411.2 |
| 817 | AW | | 20 | 465.3 |
| 818 | BC | 3880 | >10000 | 439.2 |
| 819 | AW | | 23 | 483.3 |
| 820 | L | | 229 | 406.9 |
| 821 | L, AH | | 71 | 469.3 |
| 822 | L, AH | | 172 | 469.3 |
| 823 | AK | | 36 | 477.2 |
| 824 | AU | | 2 | 473.2 |
| 825 | AU | | 9 | 491.3 |
| 826 | AU | | 12 | 503.3 |
| 827 | AZ | | 12 | 469.2 |
| 828 | AL | | 30 | 477.3 |
| 829 | AL | <3 | 10 | 473.3 |
| 830 | AL | 8 | 305 | 473.3 |
| 831 | AT | <3 | 118 | 522.2 |
| 832 | AU | | 217 | 473.3 |
| 833 | AL | | 47 | 463.2 |
| 834 | AL | 21 | 490 | 545.2 |
| 835 | AL | 11 | 86 | 545.2 |
| 836 | AL | | 15 | 475.2 |
| 837 | AL | | 22 | 475.2 |
| 838 | AL | | 55 | 476.2 |
| 839 | AT | 4 | 22 | 479.3 |
| 840 | AT | | 54 | 485.2 |
| 841 | AT | | 1120 | 464.1 |
| 842 | AU | | 3 | 505.9 |
| 843 | AT | 3 | 22 | 463.0 |
| 844 | AT | | 38 | 468.9 |
| 845 | L | 4 | 58 | 447.9 |
| 846 | AZ | | 12 | 455.2 |
| 847 | AW | | 22 | 467.0 |
| 848 | AW | | 78 | 419.0 |
| 849 | AW | | 174 | 461.0 |
| 850 | AU | | 5 | 505.9 |
| 851 | AL | | 44 | 463.2 |
| 852 | L | 17 | 502 | 402.2 |
| 853 | AL | | 538 | 476.3 |
| 854 | AU | | 88 | 524.9 |
| 855 | AV | | 13 | 523.9 |
| 856 | AT | | 35 | 451.0 |
| 857 | L, AA | | 75 | 461.9 |
| 858 | AU | | 7 | 541.9 |
| 859 | AW | | 41 | 477.9 |
| 860 | L, AH | | 232 | 447.9 |
| 861 | L, AH | | 95 | 461.9 |
| 862 | L, AH | | 121 | 461.9 |
| 863 | AL | | 2080 | 544.0 |
| 864 | AU | | 9 | 491.9 |
| 865 | AS | | 10 | 497.8 |
| 866 | BC | | 62 | 402.1 |
| 867 | AU | | 14 | 541.9 |
| 868 | AU | | 139 | 524.9 |
| 869 | AW | | 55 | 466.9 |
| 870 | AL | | 78 | 498.2 |
| 871 | AW | | 39 | 418.9 |
| 872 | AW | | 147 | 460.9 |
| 873 | AT | 19 | 862 | 530.9 |
| 874 | AW | | 121 | 477.9 |
| 875 | AW | | 170 | 508.9 |
| 876 | AL | | 734 | 526.2 |
| 877 | AU | | 37 | 521.8 |
| 878 | AU | | 36 | 523.8 |
| 879 | AK | | 7 | 509.8 |
| 880 | AL | | 8 | 497.8 |
| 881 | AL | <3 | 789 | 473.2 |
| 882 | AL | | 49 | 498.2 |
| 883 | AT | | 36 | 498.8 |
| 884 | AL | 13 | 1610 | 469.2 |
| 885 | AL | 176 | >10000 | 467.2 |
| 886 | BC | | 37 | 457.1 |
| 887 | AW | | 104 | 498.9 |
| 888 | AL | | 111 | 475.2 |
| 889 | AL | | 56 | 475.2 |
| 890 | AL | | 81 | 474.9 |
| 891 | AL | | 22 | 474.9 |
| 892 | AX | 15 | 589 | 488.0 |
| 893 | AL | <3 | 209 | 481.2 |
| 894 | AL | 17 | 447 | 444.1 |
| 895 | AL | | 876 | 473.2 |
| 896 | AX | 4 | 147 | 453.9 |
| 897 | AL | | 122 | 498.8 |
| 898 | AL | | 76 | 474.9 |
| 899 | AL | | 33 | 474.9 |
| 900 | AX | 6 | 46 | 432.0 |
| 901 | AL | <3 | 27 | 459.2 |
| 902 | AL | 619 | >10000 | 467.2 |
| 903 | AX | 4 | 195 | 465.9 |
| 904 | AX | 4 | 147 | 406.0 |
| 905 | BC | <3 | >10000 | 462.2 |
| 906 | L, AH | | 51 | 447.9 |
| 907 | AK | | 214 | 506.9 |
| 908 | L | <3 | 95 | 487.1 |
| 909 | AK | | 107 | 540.8 |
| 910 | AX | | 200 | 501.8 |
| 911 | AK | | 1 | 469.9 |
| 912 | L, AA | | 178 | 462.9 |
| 913 | AK | | 8 | 493.8 |
| 914 | L | | 54 | 401.0 |
| 915 | AX | | 164 | 467.9 |
| 916 | AY | | 926 | 441.0 |
| 917 | AK | | 203 | 473.8 |
| 918 | AL | | 47 | 445.0 |
| 919 | L | | 92 | 421.0 |
| 920 | AX | | 118 | 451.9 |
| 921 | AK | | 4 | 491.9 |
| 922 | AK | | 34 | 503.9 |
| 923 | AK | | 68 | 476.8 |
| 924 | AK | | 15 | 490.8 |
| 925 | AY | | 129 | 458.9 |
| 926 | AY | | 659 | 441.9 |
| 927 | AX | | 343 | 434.3 |
| 928 | AK | | 144 | 488.8 |
| 929 | AK | | 7 | 473.8 |
| 930 | AK | | 5 | 476.8 |
| 931 | AK | | 14 | 505.8 |
| 932 | L | | 469 | 387.0 |
| 933 | L | | 65 | 405.0 |
| 934 | AK | | 12 | 481.1 |
| 935 | AK | | 8 | 497.0 |
| 936 | BA | 2 | 221 | 502.2 |
| 937 | BA | 2 | 216 | 502.2 |
| 938 | AT | | 2 | 456.2 |
| 939 | BA | | 9 | 490.0 |
| 940 | AK | | 269 | 496.1 |
| 941 | AK | | 9 | 487.0 |
| 942 | L | | 396 | 481.0 |
| 943 | AK | | 22 | 504.0 |
| 944 | AU | | 28 | 522.0 |
| 945 | AK | | 23 | 506.1 |
| 946 | AU | | 51 | 476.1 |
| 947 | AK | | 4 | 524.0 |
| 948 | AD | | 44 | 458.1 |
| 949 | AD | | 50 | 476.0 |
| 950 | AK | | 42 | 473.0 |
| 951 | AT | | 16 | 439.2 |
| 952 | AK | | 1 | 474.1 |
| 953 | AK | | 153 | 457.0 |
| 954 | BC | | 11 | 489.1 |
| 955 | BC | | 51 | 473.1 |
| 956 | AW | | 92 | 459.2 |
| 957 | BC | | 160 | 455.2 |
| 958 | AS | | 32 | 483.0 |
| 959 | AK | | 8 | 509.1 |
| 960 | AK | | 40 | 459.2 |
| 961 | BC | | 223 | 488.1 |
| 962 | BC | | 702 | 472.2 |
| 963 | BC | | >10000 | 489.3 |
| 964 | BA | | 68 | 466.2 |
| 965 | AU | | 8 | 494.0 |
| 966 | AU | | 11 | 494.0 |
| 967 | BA | | 212 | 439.0 |
| 968 | AS | | 4 | 484.0 |
| 969 | AK | | 16 | 485.0 |
| 970 | AK | | 40 | 510.0 |
| 971 | AK | | 21 | 478.1 |
| 972 | AK | | 14 | 492.1 |
| 973 | AK | | 53 | 524.1 |
| 974 | BB | | 186 | 482.1 |
| 975 | BB | | 14 | 484.3 |
| 976 | BC | | 22 | 486.2 |
| 977 | BB | | 203 | 514.1 |

### Example 3. Genetic Validation

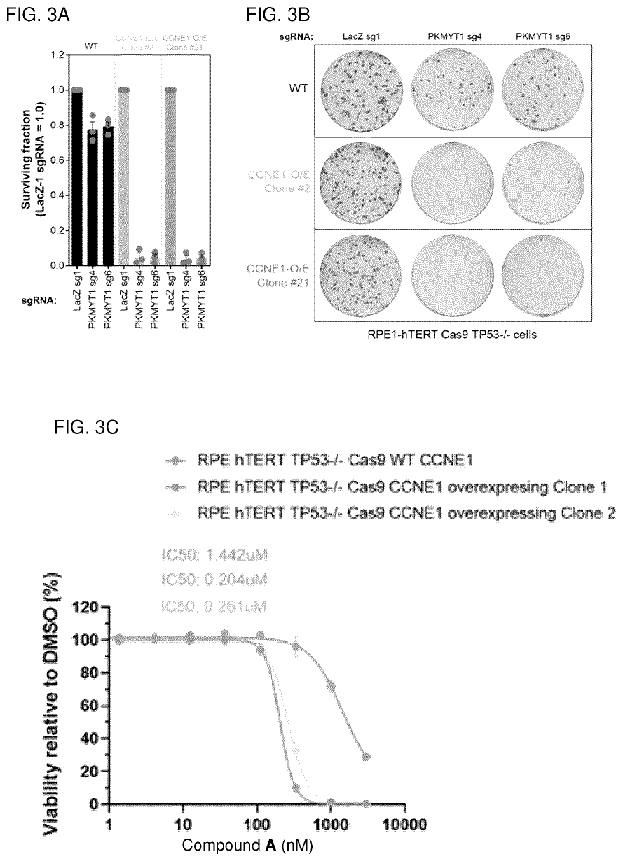

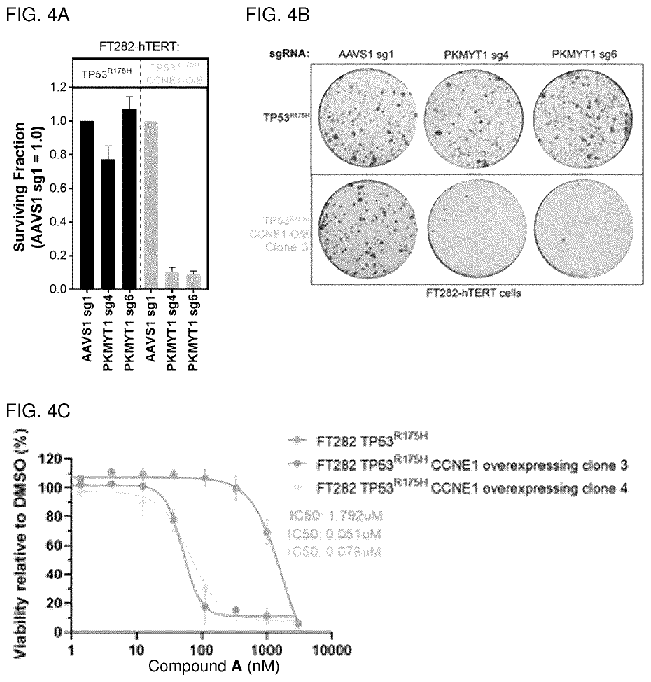

Two sgRNAs for PKMYT1 and one sgRNA for LacZ (control) were transduced into the RPE1-hTERT Cas9 TP53−/− parental (WT) and CCNE1-overexpressing clones. Infected cells were plated at low density to measure their ability to form colonies of <50 cells. After 10 days of growth, the colonies were stained, imaged, and quantified. Using clonogenic survival assays, we observed a profound cellular fitness defect in CCNE1-overexpressing cells compared to parental cells transduced with PKMYT1 sgRNAs (FIGS. 3A and 3B). This experiment was repeated using FT282-hTERT TP53−/− parental (WT) and CCNE1-overexpressing clones and similar results were observed (FIGS. 4A and 4B).

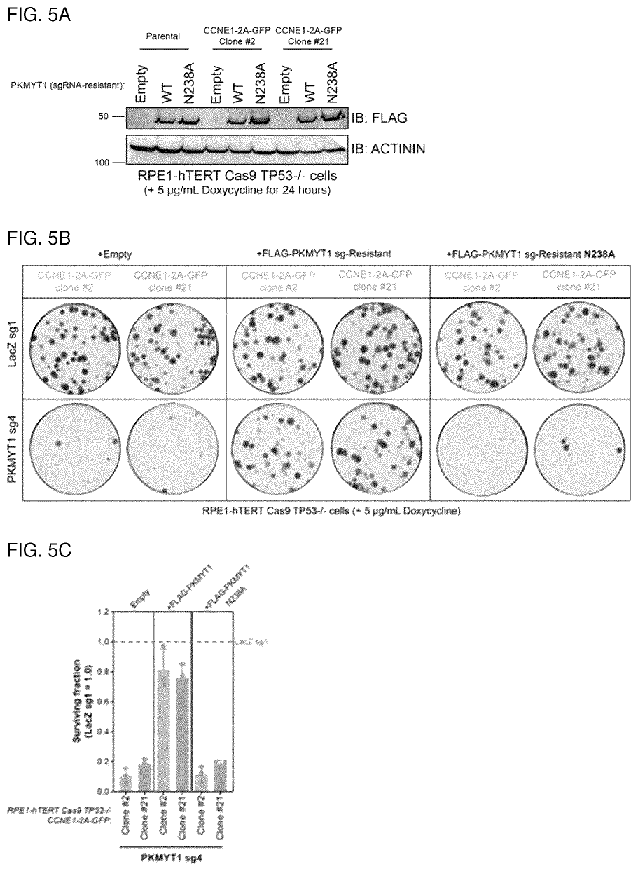

To determine if the kinase activity of PKMYT1 was responsible for maintaining the viability of CCNE1-overexpressing RPE1-hTERT Cas9 TP53−/− cells, the PKMYT1 open reading frame (ORF) was cloned into an inducible mammalian expression vector. sgRNA-resistant silent mutations in the PKMYT1 ORF sequence were then created by PCR mutagenesis. A single point mutation was generated that resulted in an asparagine (N) to alanine (A) amino acid change at residue 238. The N238A amino acid change in the kinase domain resulted in a catalytically inactive PKMYT1 mutant. Stable cell lines in the RPE1-hTERT Cas9 TP53−/− parental and CCNE1-overexpressing clones were generated that either expressed the wild type PKMYT1 ORF or the kinase-dead N238A mutant (FIG. 5A). These stable cell lines were transduced with either a LacZ non-targeting sgRNA or PKMYT1 sgRNA #4. The cells were then plated at low density to measure their ability to form colonies of >50 cells. After 10 days of growth, the colonies were stained, imaged, and quantified. Expression of an sgRNA-resistant PKMYT1 ORF but not the catalytic-dead version rescued the fitness defect induced by transduction of sgRNA #4 into both CCNE1-overexpressing clones (FIGS. 5B and 5C). This result demonstrated that targeting the kinase activity of PKMYT1 selectively kills CCNE1-overexpressing cells.

### Example 4. Pharmacological Validation

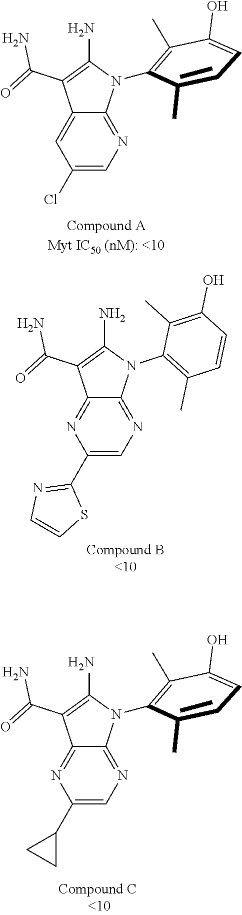

RPE1-hTERT Cas9 TP53−/− parental (WT) and CCNE1-overexpressing clones were treated with compound A in a dose titration and cell viability was determined. The CCNE1-overexpressing cells were found to be more sensitive to compound A than the corresponding WT cells (FIG. 3C). A similar effect was seen in FT282-hTERT TP53^{R175H} WT and CCNE1-overexpressing clones (FIG. 4C). For dose-response proliferation assays using RPE1-hTERT and FT282-hTERT cell lines, cells were seeded in 96-well plates and dosed with serially diluted Myt1 inhibitor. Cells were imaged once per day using the IncuCyte S₃ microscope and percent well confluency was calculated over time. Once cells reached four population doublings the experiment was ended and IC₅₀ curves were plotted for the final time point. Percentage confluency was calculated relative to the cell confluency in the untreated wells.

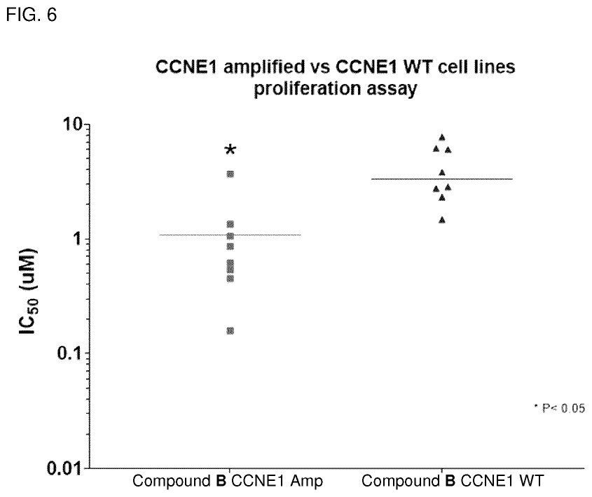

A panel of 16 cancer cell lines with either normal (n=8) or elevated levels of CCNE1 (n=8) was evaluated for their sensitivity to compound B in a cell proliferation assay (FIG. 6). Dose-response curves in these cancer cell line proliferation assays were generated as follows. Cells were seeded in 96-well plates and dosed with serially diluted compound B. After 7 days, Cell Titer Glo (CTG) was used to assess the proliferation status of these cells and the IC₅₀ values were plotted.

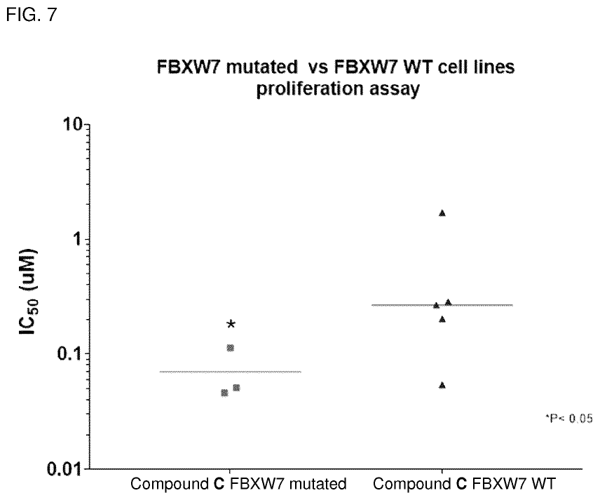

A similar experiment was conducted in a panel of 8 cancer cell lines with either wild-type FBXW7 (n=5) or FBXW7-mutations (n=3) in which these cells were evaluated for their sensitivity to compound C in a cell proliferation assay (FIG. 7). Dose-response curves in these cancer cell line proliferation assays were generated as follows. Cells were seeded in 96-well plates and dosed with serially diluted Myt1 inhibitor. Cells were imaged once per day using the IncuCyte S₃ microscope and percent well confluency was calculated over time. Once cells reached four population doublings the experiment was ended and IC₅₀ curves were plotted for the final time point. Percentage confluency was calculated relative to the cell confluency in the untreated wells.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A compound of formula (I): 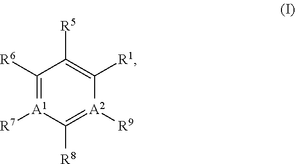
or a pharmaceutically acceptable salt thereof,
wherein
R¹ is: 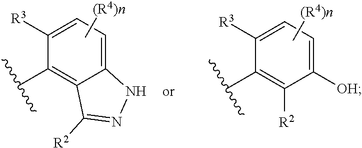
n is 0, 1, or 2;
each of R² and R³ is independently hydrogen, halogen, optionally substituted C₃₋₄ cycloalkyl, or optionally substituted C₁₋₆ alkyl;
each R⁴ is independently halogen;
R⁵ is hydrogen, halogen, hydroxyl, optionally substituted C₁₋₆ alkyl, optionally substituted C₁₋₆ alkoxy, or —N(R^{5A})₂;
each R^{5A} is independently hydrogen, optionally substituted C₁₋₆ alkyl or optionally substituted C₃₋₈ cycloalkyl;
R⁶ is —C(O)NH(R^{6A}), —SO₂R^{6B}, or —C(O)R^{6C};
R^{6A} is hydrogen, optionally substituted C₁₋₆ alkyl, optionally substituted C₃₋₈ cycloalkyl, optionally substituted C₂₋₉ heterocyclyl, optionally substituted C₆₋₁₀ aryl, or optionally substituted C₁₋₉ heteroaryl;
R^{6B} is optionally substituted C₁₋₆ alkyl, optionally substituted C₃₋₈ cycloalkyl, optionally substituted C₆₋₁₀ aryl, or —NH(R^{6A});
R^{6C} is optionally substituted C₁₋₆ alkyl;
each of A¹ and A² is independently N or C;
R⁷ is absent, hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, or —OR¹⁰; R⁸ is hydrogen, halogen, optionally substituted C₁₋₆ alkyl, optionally substituted C₂₋₆ alkenyl, optionally substituted C₂₋₃ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR¹⁰, —N(R¹¹)₂, or -L-R^{8A}; or R⁷ and R⁸ combine with the atoms to which they are attached to form an optionally substituted C₃₋₈ cycloalkenyl, optionally substituted C₂₋₉ heterocyclyl, optionally substituted C₆₋₁₀ aryl, or optionally substituted C₂₋₁₂ heteroaryl; and R⁹ is absent, hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, or —OR¹⁰;
or
R⁸ is hydrogen, halogen, optionally substituted C₁₋₆ alkyl, optionally substituted C₂₋₆ alkenyl, optionally substituted C₂₋₃ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR¹⁰, —N(R¹¹)₂, or -L-R^{3A}; and R⁹ is absent, hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, or —OR¹⁰; or R⁸ and R⁹ combine with the atoms to which they are attached to form an optionally substituted C₃₋₈ cycloalkenyl, optionally substituted C₂₋₉ heterocyclyl, optionally substituted C₆₋₁₀ aryl, or optionally substituted C₁₋₁₂ heteroaryl; and R⁷ is absent, hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, or —OR¹⁰;
L is optionally substituted C₂₋₉ heterocyclylene, optionally substituted C₂₋₉ heteroarylene, optionally substituted C₆₋₁₀ arylene, or optionally substituted C₃₋₈ cycloalkylene;
R^{3A} is hydrogen, halogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR¹⁰, or —N(R¹¹)₂;
R¹⁰ is hydrogen, optionally substituted C₁₋₆ alkyl, optionally substituted C₁₋₃ heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R¹¹ is independently hydrogen, halogen, optionally substituted C₁₋₆ alkyl, optionally substituted acyl, optionally substituted C₁₋₃ heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl, —SO₂R^{11A} or two R¹¹ groups combine to form an optionally substituted C₂₋₉ heterocyclyl; and
each R^{11A} is independently hydrogen, optionally substituted C₁₋₆ alkyl, optionally substituted C₂₋₆ alkenyl, optionally substituted C₂₋₆ alkynyl, optionally substituted C₁₋₃ heteroalkyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl.

2. The compound of claim 1, or a pharmaceutically acceptable salt thereof, wherein R⁵ is N(R^{5A})₂.

3. The compound of claim 1 or 2, or a pharmaceutically acceptable salt thereof, wherein each R^{5A} is hydrogen.

4. The compound of any one of claims 1 to 3, or a pharmaceutically acceptable salt thereof, wherein R¹ is

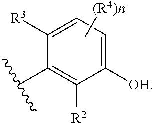

5. The compound of claim 1, or a pharmaceutically acceptable salt thereof, wherein the compound is of formula (II-A):

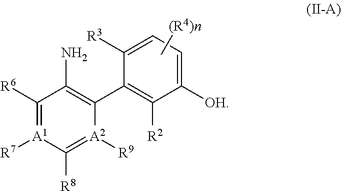

6. The compound of claim 1, or a pharmaceutically acceptable salt thereof, wherein the compound is of formula (II-A-i):

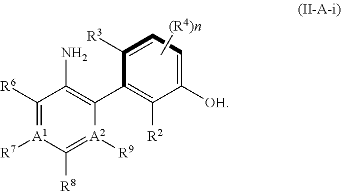

7. The compound of any one of claims 1 to 3, or a pharmaceutically acceptable salt thereof, wherein R¹ is

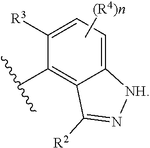

8. The compound of claim 1, or a pharmaceutically acceptable salt thereof, wherein the compound is of formula (II-B):

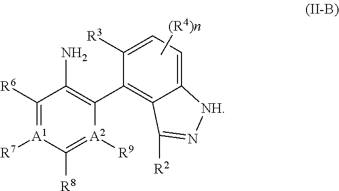

9. The compound of claim 1, or a pharmaceutically acceptable salt thereof, wherein the compound is of formula (II-B-i):

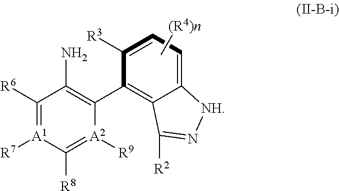

10. The compound of any one of claims 1 to 9, or a pharmaceutically acceptable salt thereof, wherein A¹ is C.

11. The compound of any one of claims 1 to 10, or a pharmaceutically acceptable salt thereof, wherein R⁷ is hydrogen.

12. The compound of any one of claims 1 to 10, or a pharmaceutically acceptable salt thereof, wherein R⁷ and R⁸ combine with the atoms to which they are attached to form an optionally substituted C₂₋₁₂ heteroaryl.

13. The compound of any one of claims 1 to 12, or a pharmaceutically acceptable salt thereof, wherein A² is C.

14. The compound of any one of claims 1 to 13, or a pharmaceutically acceptable salt thereof, wherein R⁸ is hydrogen.

15. The compound of any one of claims 1 to 12, or a pharmaceutically acceptable salt thereof, wherein A² is N.

16. The compound of any one of claims 1 to 9 and 13 to 15, or a pharmaceutically acceptable salt thereof, wherein A¹ is N.

17. The compound of claim 1 or a pharmaceutically acceptable salt thereof, wherein the compound is of formula (II-B-a):

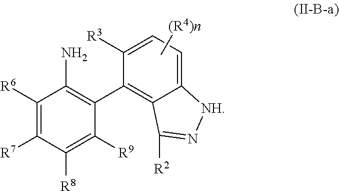

18. The compound of claim 1 or a pharmaceutically acceptable salt thereof, wherein the compound is of formula (II-B-b):

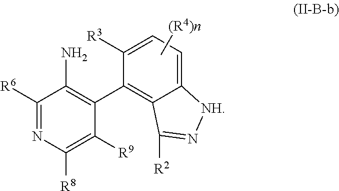

19. The compound of claim 1 or a pharmaceutically acceptable salt thereof, wherein the compound is of formula (II-B-c):

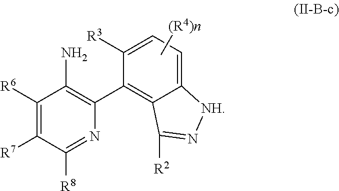

20. The compound of claim 1 or a pharmaceutically acceptable salt thereof, wherein the compound is of formula (II-B-d):

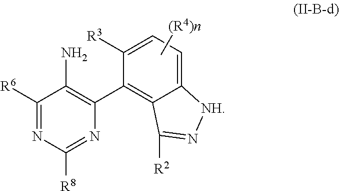

21. The compound of claim 1 or a pharmaceutically acceptable salt thereof, wherein the compound is of formula (II-A-a):

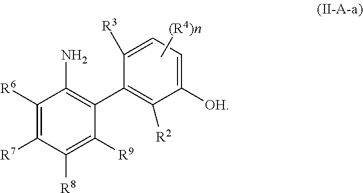

22. The compound of claim 1 or a pharmaceutically acceptable salt thereof, wherein the compound is of formula (II-A-b):

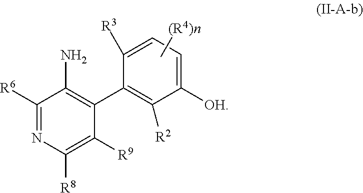

23. The compound of claim 1 or a pharmaceutically acceptable salt thereof, wherein the compound is of formula (II-A-c):

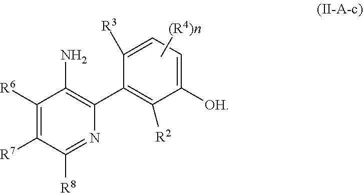

24. The compound of claim 1 or a pharmaceutically acceptable salt thereof, wherein the compound is of formula (II-A-d):

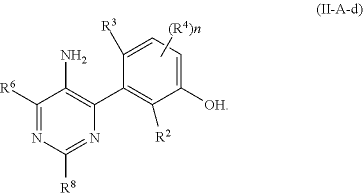

25. The compound of claim 1, or a pharmaceutically acceptable salt thereof, wherein the compound is of formula (II-C):

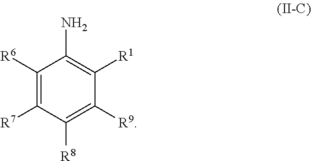

26. The compound of claim 1, or a pharmaceutically acceptable salt thereof, wherein the compound is of formula (II-D):

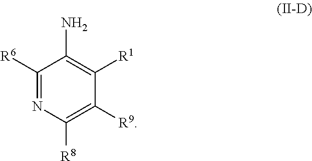

27. The compound of claim 1, or a pharmaceutically acceptable salt thereof, wherein the compound is of formula (II-E):

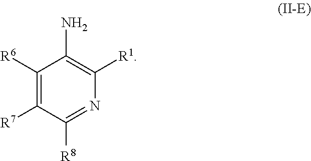

28. The compound of any one of claims 1 to 6, or a pharmaceutically acceptable salt thereof, wherein the compound is of formula (II-F):

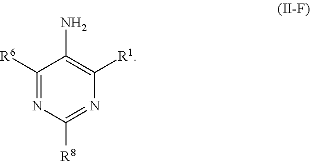

29. The compound of any one of claims 1 to 28, or a pharmaceutically acceptable salt thereof, wherein R⁶ is —C(O)NH(R^{6A}).

30. The compound of any one of claims 1 to 29, or a pharmaceutically acceptable salt thereof, wherein each R^{6A} is H.

31. The compound of any one of claims 1 to 30, or a pharmaceutically acceptable salt thereof, wherein R² is H.

32. The compound of any one of claims 1 to 31, or a pharmaceutically acceptable salt thereof, wherein R³ is H.

33. The compound of any one of claims 1 to 30, or a pharmaceutically acceptable salt thereof, wherein one of R² and R³ is H and the other is optionally substituted C₁₋₆ alkyl.

34. The compound of claim 33, or a pharmaceutically acceptable salt thereof, wherein one of R² and R³ is H and the other is —CH₃.

35. The compound of any one of claims 1 to 30, or a pharmaceutically acceptable salt thereof, wherein R² and R³ are each optionally substituted C₁₋₆ alkyl.

36. The compound of claim 35, or a pharmaceutically acceptable salt thereof, wherein R² and R³ are each —CH₃.

37. The compound of any one of claims 1 to 30 and 32, or a pharmaceutically acceptable salt thereof, wherein R² is halogen.

38. The compound of claim 37, or a pharmaceutically acceptable salt thereof, wherein R² is Cl.

39. The compound of claim 37, or a pharmaceutically acceptable salt thereof, wherein R² is F.

40. The compound of any one of claims 1 to 31 and 37 to 39, or a pharmaceutically acceptable salt thereof, wherein R³ is halogen.

41. The compound of claim 40, or a pharmaceutically acceptable salt thereof, wherein R³ is C₁.

42. The compound of claim 40, or a pharmaceutically acceptable salt thereof, wherein R³ is F.

43. The compound of any one of claims 1 to 42, or a pharmaceutically acceptable salt thereof, wherein n is 0.

44. The compound of any one of claims 1 to 42, or a pharmaceutically acceptable salt thereof, wherein n is 1.

45. The compound of any one of claims 1 to 42 and 44, or a pharmaceutically acceptable salt thereof, wherein R⁴ is halogen.

46. The compound of claim 45, or a pharmaceutically acceptable salt thereof, wherein R⁴ is F.

47. The compound of any one of claims 1 to 3, 7 to 20, 25 to 31, 33, 34, and 43, or a pharmaceutically acceptable salt thereof, wherein R¹ is:

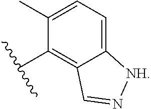 [SMILES: *c1c(C)ccc2[nH]ncc12]

48. The compound of any one of claims 1 to 11, 13, and 15 to 47, or a pharmaceutically acceptable salt thereof, wherein R⁸ is optionally substituted C₆₋₁₀ aryl.

49. The compound of claim 48, or a pharmaceutically acceptable salt thereof, wherein R⁸ is optionally substituted phenyl.

50. The compound of claim any one of claims 1 to 11, 13, and 15 to 47, or a pharmaceutically acceptable salt thereof, wherein R⁸ is optionally substituted C₁₋₉ heteroaryl.

51. The compound of any one of claims 1 to 11, 13, and 15 to 47, wherein R⁸ is -L-R^{8A}.

52. The compound of claim 51, or a pharmaceutically acceptable salt thereof, wherein L is optionally substituted pyrimidinyl.

53. The compound of claim 51, or a pharmaceutically acceptable salt thereof, wherein L is optionally substituted pyridyl.

54. The compound of claim 51, or a pharmaceutically acceptable salt thereof, wherein L is optionally substituted indazolyl.

55. The compound of claim 51, or a pharmaceutically acceptable salt thereof, wherein L is optionally substituted pyrazolyl.

56. The compound of claim 51, or a pharmaceutically acceptable salt thereof, wherein L is optionally substituted imidazolyl.

57. The compound of claim 51, or a pharmaceutically acceptable salt thereof, wherein L is optionally substituted thiazolyl.

58. The compound of claim 51, or a pharmaceutically acceptable salt thereof, wherein L is optionally substituted pyridazinyl.

59. The compound of claim 51, or a pharmaceutically acceptable salt thereof, wherein L is optionally substituted indolyl.

60. The compound of claim 51, or a pharmaceutically acceptable salt thereof, wherein L is optionally substituted furyl.

61. The compound of claim 50, or a pharmaceutically acceptable salt thereof, wherein R⁸ is an optionally substituted bicyclic heteroaryl.

62. The compound of claim 51, or a pharmaceutically acceptable salt thereof, wherein -L-R^{3A} is: 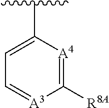
wherein each of A³ and A⁴ is independently N or CH.

63. The compound of claim 62, or a pharmaceutically acceptable salt thereof, wherein A³ is N.

64. The compound of claim 62 or 63, or a pharmaceutically acceptable salt thereof, wherein A⁴ is N.

65. The compound of claim 62 or 64, or a pharmaceutically acceptable salt thereof, wherein A³ is CH.

66. The compound of any one of claims 62, 63, and 65, or a pharmaceutically acceptable salt thereof, wherein A⁴ is CH.

67. The compound of any one of claims 62 to 66, or a pharmaceutically acceptable salt thereof, wherein R^{8A} is —OR¹⁰.

68. The compound of claim 67, or a pharmaceutically acceptable salt thereof, wherein R¹⁰ is optionally substituted C₁₋₆ alkyl.

69. The compound of claim 67, or a pharmaceutically acceptable salt thereof, wherein R¹⁰ is —CH₃.

70. The compound of claim 67, or a pharmaceutically acceptable salt thereof, wherein R¹⁰ is optionally substituted C₂₋₉ heterocyclyl.

71. The compound of claim 67, or a pharmaceutically acceptable salt thereof, wherein R¹⁰ is optionally substituted C₆₋₁₀ aryl.

72. The compound of any one of claims 62 to 66, or a pharmaceutically acceptable salt thereof, wherein R^{8A} is —N(R¹¹)₂.

73. The compound of claim 72, or a pharmaceutically acceptable salt thereof, wherein one R¹¹ is H.

74. The compound of claim 72, or a pharmaceutically acceptable salt thereof, wherein one R¹¹ is optionally substituted C₁₋₆ alkyl.

75. The compound of claim 72, or a pharmaceutically acceptable salt thereof, wherein each R¹¹ is H.

76. The compound of claim 72, or a pharmaceutically acceptable salt thereof, wherein two R¹¹ groups combine to form an optionally substituted C₂₋₉ heterocyclyl.

77. The compound of claim 51, or a pharmaceutically acceptable salt thereof, wherein -L-R^{8A} is: 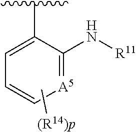
wherein each R¹⁴ is independently cyano, halogen, optionally substituted C₁₋₆ alkyl, —S(O)₂R^{14A} or optionally substituted C₁₋₈ heteroalkyl;
R^{14A} is optionally substituted C₁₋₆ alkyl;
A⁵ is N or CH; and
p is 0, 1, 2, 3, or 4.

78. The compound of claim 77, or a pharmaceutically acceptable salt thereof, wherein R¹¹ is optionally substituted acyl.

79. The compound of claim 78, or a pharmaceutically acceptable salt thereof, wherein -L-R^{8A} is: 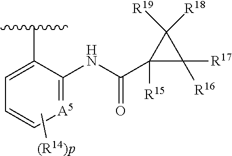
wherein each of R¹⁵, R¹⁶, R¹⁷, R¹⁸, and R¹⁹ is independently cyano, hydrogen, halogen, —CH₃, —CF₃, or —OCH₃.

80. The compound of claim 79, or a pharmaceutically acceptable salt thereof, wherein -L-R^{8A} is

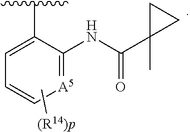

81. The compound of claim 77, or a pharmaceutically acceptable salt thereof, wherein R¹¹ is optionally substituted C₁₋₉ heteroaryl.

82. The compound of claim 81, or a pharmaceutically acceptable salt thereof, wherein the optionally substituted C₁₋₉ heteroaryl is a 6-membered heteroaryl ring containing at least one nitrogen.

83. The compound of claim 82, or a pharmaceutically acceptable salt thereof, wherein the 6-membered heteroaryl ring contains exactly 2 nitrogens.

84. The compound of claim 83, or a pharmaceutically acceptable salt thereof, wherein R¹¹ is: 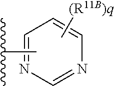
wherein each R^{11B} is independently halogen or optionally substituted C₁₋₆ alkyl; and
q is 0, 1, 2, or 3.

85. The compound of claim 84, or a pharmaceutically acceptable salt thereof, wherein q is 0.

86. The compound of claim 84, or a pharmaceutically acceptable salt thereof, wherein q is 2.

87. The compound of claim 77, or a pharmaceutically acceptable salt thereof, wherein R¹¹ is optionally substituted C₆₋₁₀ aryl.

88. The compound of claim 87, wherein R¹¹ is optionally substituted phenyl.

89. The compound of claim 87, or a pharmaceutically acceptable salt thereof, wherein R¹¹ is optionally substituted C₂₋₉ heterocyclyl.

90. The compound of claim 77, wherein R¹¹ is —S(O)₂R^{11A}.

91. The compound of any one of claims 77 to 90, or a pharmaceutically acceptable salt thereof, wherein p is 0.

92. The compound of any one of claims 77 to 90, or a pharmaceutically acceptable salt thereof, wherein p is 1.

93. The compound of any one of claims 77 to 90, or a pharmaceutically acceptable salt thereof, wherein p is 2.

94. The compound of any one of claims 77 to 90, 92, and 93, or a pharmaceutically acceptable salt thereof, wherein each R¹⁴ is independently halogen.

95. The compound of claim 94, or a pharmaceutically acceptable salt thereof, wherein each R¹⁴ is F.

96. The compound of any one of claims 77 to 95, or a pharmaceutically acceptable salt thereof, wherein A⁵ is CH.

97. The compound of any one of claims 77 to 95, or a pharmaceutically acceptable salt thereof, wherein A⁵ is N.

98. The compound of any one of claims 77 to 90, 92, and 94 to 97, or a pharmaceutically acceptable salt thereof, wherein -L-R^{8A} is:

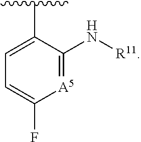

99. The compound of any one of claims 1 to 47, or a pharmaceutically acceptable salt thereof, wherein R⁸ is optionally substituted C₃₋₈ cycloalkyl.

100. A compound selected from the group consisting of compounds 1 to 977 and pharmaceutically acceptable salts thereof.

101. A pharmaceutical composition comprising the compound of any one of claims 1 to 100, or a pharmaceutically acceptable salt thereof, and a pharmaceutically acceptable excipient.

102. The pharmaceutical composition of claim 101, wherein the composition is isotopically enriched in deuterium.

103. A method of treating a cancer in a subject, the method comprising administering to the subject in need thereof a therapeutically effective amount of the compound of any one of claims 1 to 100, or a pharmaceutically acceptable salt thereof, or the pharmaceutical composition of claim 101 or 102, wherein the cancer has been previously identified as a cancer overexpressing CCNE1.

104. A method of treating a cancer in a subject, the method comprising administering to the subject in need thereof a therapeutically effective amount of the compound of any one of claims 1 to 100, or a pharmaceutically acceptable salt thereof, or the pharmaceutical composition of claim 101 or 102, wherein the cancer is a cancer overexpressing CCNE1.

105. A method of inducing cell death in a cancer cell overexpressing CCNE1, the method comprising contacting the cell with an effective amount of the compound of any one of claims 1 to 100, or a pharmaceutically acceptable salt thereof.

106. The method of any one of claims 103 to 105, wherein the cancer is uterine cancer, ovarian cancer, breast cancer, stomach cancer, esophageal cancer, lung cancer, or endometrial cancer.

107. A method of treating a cancer in a subject, the method comprising administering to the subject in need thereof a therapeutically effective amount of the compound of any one of claims 1 to 100, or a pharmaceutically acceptable salt thereof, or the pharmaceutical composition of claim 101 or 102, wherein the cancer has been previously identified as a cancer having an inactivating mutation in the FBXW7 gene.

108. A method of treating a cancer in a subject, the method comprising administering to the subject in need thereof a therapeutically effective amount of the compound of any one of claims 1 to 100, or a pharmaceutically acceptable salt thereof, or the pharmaceutical composition of claim 101 or 102, wherein the cancer has an inactivating mutation in the FBXW7 gene.

109. A method of inducing cell death in an FBXW7-mutated cancer cell, the method comprising contacting the cell with an effective amount of the compound of any one of claims 1 to 100, or a pharmaceutically acceptable salt thereof.

110. The method of any one of claims 107 to 109, wherein the cancer is uterine cancer, colorectal cancer, breast cancer, lung cancer, or esophageal cancer.

111. The method of claim 105, 106, 109, or 110, wherein the cell is in a subject.
